(19) United States
(12) Patent Application Publication
(10) Pub. No.: US 2021/0189342 A1
(43) Pub. Date: Jun. 24, 2021

(54) COMPOSITIONS AND METHODS FOR MODULATING MONOCYTE AND MACROPHAGE INFLAMMATORY PHENOTYPES AND IMMUNOTHERAPY USES THEREOF

(71) Applicant: Verseau Therapeutics, Inc., Bedford, MA (US)

(72) Inventors: Tatiana I. Novobrantseva, Wellesley, MA (US); Igor Feldman, Waltham, MA (US)

(21) Appl. No.: 17/252,379

(22) PCT Filed: Jun. 28, 2019
(86) PCT No.: PCT/US19/39773

§ 371 Date: Dec. 15, 2020

Related U.S. Application Data
(60) Provisional application No. 62/867,532, filed on Jun. 27, 2019.
(60) Provisional application No. 62/857,199, filed on Jun. 4, 2019.
(60) Provisional application No. 62/810,683, filed on Feb. 26, 2019.
(60) Provisional application No. 62/692,463, filed on Jun. 29, 2018.

(51) Int. Cl. C12N 5/0786; C12N 15/113; C07K 16/28; A61K 35/15; G01N 33/50; G01N 33/574; A61K 45/06; A61K 31/495; A61K 9/51

(52) U.S. Cl. CPC C12N 5/0645; C12N 15/113; C07K 16/2818; A61K 35/15; G01N 33/5005; G01N 33/574; C12N 2310/315; G01N 33/5055; G01N 33/5011; A61K 31/495; A61K 9/5123; C12N 2310/14; A61K 45/06

(57) ABSTRACT

The present invention is based, in part, on the identification of compositions and methods for modulating monocyte and macrophage inflammatory phenotypes and immunotherapy uses thereof.

### CROSS-REFERENCE TO RELATED APPLICATIONS

This application claims the benefit of U.S. Provisional Application No. 62/692,463 filed on 29 Jun. 2018, U.S. Provisional Application No. 62/810,683 filed on 26 Feb. 2019, U.S. Provisional Application No. 62/857,199 filed on 4 Jun. 2019, and U.S. Provisional Application No. 62/867,532 filed on 27 Jun. 2019; the entire contents of each of said applications is incorporated herein in their entirety by this reference.

### BACKGROUND OF THE INVENTION

Monocytes and macrophages are types of phagocytes, which are cells that protect the body by ingesting harmful foreign particles, bacteria, and dead or dying cells. In addition to monocytes and macrophages, phagocytes include neutrophils, dendritic cells, and mast cells.

Macrophages are classically known as large white blood cells that patrol the body and engulf and digest cellular debris, and foreign substances, such as pathogens, microbes, and cancer cells, through a process known as phagocytosis. In addition, macrophages, including tissue macrophages and circulating monocyte-derived macrophages, are important mediators of both the innate and adaptive immune system.

Macrophage phenotype is dependent on activation via a classical or an alternative pathway (see, e.g., Classen et al. (2009) Methods Mol. Biol. 531:29-43). Classically activated macrophages are activated by interferon gamma (IFNγ) or lipopolysaccharide (LPS) and display an M1 phenotype. This pro-inflammatory phenotype is associated with increased inflammation and stimulation of the immune system. Alternatively activated macrophages are activated by cytokines like IL-4, IL-10, and IL-13, and display an M2 phenotype. This anti-inflammatory phenotype is associated with decreased immune response, increased wound healing, increased tissue repair, and embryonic development.

Under non-pathological conditions, a balanced population of immune-stimulatory and immune-regulatory macrophages exists in the immune system. Perturbation of the balance can result in a variety of disease conditions. In some cancers, for example, tumors secrete immune factors (e.g., cytokines and interleukins) that polarize macrophage populations in favor of the anti-inflammatory, pro-tumorigenic M2 phenotype, which activates wound-healing pathways, promotes the growth of new blood vessels (i.e., angiogenesis), and provides nutrients and growth signals to the tumor. These M2 macrophages are referred to as tumor associated macrophages (TAMs), or tumor infiltrating macrophages. TAMs in the tumor microenvironment are important regulators of cancer progression and metastasis (Pollard (2004) Nat. Rev. Cancer 4:71-78). Small molecules and monoclonal antibodies designed to inhibit macrophage gene targets (e.g., CSF1R and CCR2) have been investigated as modulators of macrophage phenotypes, such as by modulating the balance of pro-tumorigenic macrophages (e.g., TAMs) and pro-inflammatory macrophages that can inhibit tumorigenesis. Therapies that modulate the recruitment, polarization, activation, and/or function of monocytes and macrophages in order to modulate the balance of macrophage populations are referred to as macrophage immunotherapies. Despite advances in the field of macrophage biology, however, there remains a need for new targets (e.g. genes and/or gene products) for modulating the inflammatory phenotype of macrophages and agents for use in macrophage immunotherapy.

### SUMMARY OF THE INVENTION

The present invention is based, at least in part, on the discovery that the inflammatory phenotype of monocytes and/or macrophages can be regulated by modulating the copy number, amount, and/or activity of one or more biomarkers described herein (e.g., targets listed in Table 1, Table 2, Examples, etc.) and uses of the biomarkers and/or modulatory agents thereof for treating, diagnosing, prognosing, and screening purposes.

For example, in one aspect, a method of generating monocytes and/or macrophages having an increased inflammatory phenotype after contact with at least one agent comprising contacting monocytes and/or macrophages with an effective amount of the at least one agent, wherein the at least one agent is a) an agent that downregulates the copy number, amount, and/or activity of at least one target listed in Table 1 and/or b) an agent that upregulates the copy number, amount, and/or activity of at least one target listed in Table 2, is provided.

Numerous embodiments are further provided that can be applied to any aspect of the present invention and/or combined with any other embodiment described herein. For example, in one embodiment, the monocytes and/or macrophages having an increased inflammatory phenotype exhibit one or more of the following after contact with the agent or agents: a) increased expression and/or secretion of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α); b) decreased expression and/or secretion of CD206, CD163, CD16, CD53, VSIG4, PSGL-1, TGFb and/or IL-10; c) increased secretion of at least one cytokine or chemokine selected from the group consisting of IL-1β, TNF-α, IL-12, IL-18, GM-CSF, CCL3, CCL4, and IL-23; d) increased ratio of expression of IL-IP, IL-6, and/or TNF-α to expression of IL-10; e) increased CD8+ cytotoxic T cell activation; f) increased recruitment of CD8+ cytotoxic T cell activation; g) increased CD4+ helper T cell activity; h) increased recruitment of CD4+ helper T cell activity; i) increased NK cell activity; j) increased recruitment of NK cell; k) increased neutrophil activity; l) increased macrophage activity; and/or m) increased spindle-shaped morphology, flatness of appearance, and/or number of dendrites, as assessed by microscopy. In another embodiment, the monocytes and/or macrophages contacted with the agent or agents are comprised within a population of cells and the agent increase the number of Type 1 and/or M1 macrophages, and/or decrease the number of Type 2 and/or M2 macrophages, in the population of cells. In still another embodiment, the monocytes and/or macrophages contacted with the agent or agents are comprised within a population of cells and the agent or agents increases the ratio of i) to ii), wherein i) is Type 1 and/or M1 macrophages and ii) is Type 2 and/or M2 macrophages in the population of cells.

In another aspect, a method of generating monocytes and/or macrophages having a decreased inflammatory phenotype after contact with at least one agent comprising contacting monocytes and/or macrophages with an effective amount of the at least one agent, wherein the agent is a) an agent that upregulates the copy number, amount, and/or activity of at least one target listed in Table 1 and/or b) an agent that downregulates the copy number, amount, and/or activity of at least one target listed in Table 2, is provided.

As described above, numerous embodiments are further provided that can be applied to any aspect of the present invention and/or combined with any other embodiment described herein. For example, in one embodiment, the monocytes and/or macrophages having the decreased inflammatory phenotype exhibit one or more of the following after contact with the agent or agents: a) decreased expression and/or secretion of cluster of differentiation 80 (CD80), CD86, MHCII, MHCI, interleukin 1-beta (IL-1β), IL-6, CCL3, CCL4, CXCL 10, CXCL9, GM-CSF and/or tumor necrosis factor alpha (TNF-α); b) increased expression and/or secretion of CD206, CD163, CD16, CD53, VSIG4, PSGL-1 and/or IL-10; c) decreased secretion of at least one cytokine selected from the group consisting of IL-1β, TNF-α, IL-12, IL-18, and IL-23; d) decreased ratio of expression of IL-1β, IL-6, and/or TNF-α to expression of IL-10; e) decreased CD8+ cytotoxic T cell activation; f) decreased CD4+ helper T cell activity; g) decreased NK cell activity; h) decreased pro-inflammatory neutrophil activity; i) decreased macrophage activity; and/or j) decreased spindle-shaped morphology, flatness of appearance, and/or number of dendrites, as assessed by microscopy. In another embodiment, the monocytes and/or macrophages contacted with the agent or agents are comprised within a population of cells and the agent decrease the number of Type 1 and/or M1 macrophages, and/or increase the number of Type 2 and/or M2 macrophages, in the population of cells. In still another embodiment, the monocytes and/or macrophages contacted with the agent or agents are comprised within a population of cells and the agent or agents decrease the ratio of i) to ii), wherein i) is Type 1 and/or M1 macrophages and ii) is Type 2 and/or M2 macrophages in the population of cells. In yet another embodiment, the agent or agents that downregulate the copy number, amount, and/or activity of at least one target listed in Table 1 and/or Table 2 is a small molecule inhibitor, CRISPR guide RNA (gRNA), RNA interfering agent, antisense oligonucleotide, single-stranded nucleic acid, double-stranded nucleic acid, aptamer, ribozyme, DNAzyme, peptide, peptidomimetic, antibody, intrabody, or cells. The RNA interfering agent may comprise or be, e.g., a small interfering RNA (siRNA), a small hairpin RNA (shRNA), microRNA (miRNA), or a piwi-interacting RNA (piRNA). In another embodiment, the agent or agents that downregulate the copy number, amount, and/or activity of at least one target listed in Table 1 and/or Table 2 comprises an antibody and/or intrabody, or an antigen binding fragment thereof, which specifically binds to the at least one target listed in Table 1 and/or Table 2. In still another embodiment, the antibody and/or intrabody, or antigen binding fragment thereof, is camelid, murine, chimeric, humanized, human, detectably labeled, comprises an effector domain, comprises an Fc domain, and/or is selected from the group consisting of Fv, Fav, F(ab′)2, Fab′, dsFv, scFv, sc(Fv)2, and diabodies fragments. In yet another embodiment, the antibody and/or intrabody, or antigen binding fragment thereof, is conjugated to a cytotoxic agent. In another embodiment, the cytotoxic agent is selected from the group consisting of a chemotherapeutic agent, a biologic agent, a toxin, and a radioactive isotope. In still another embodiment, the agent or agents that upregulate the copy number, amount, and/or activity of at least one target listed in Table 1 and/or Table 2 is a nucleic acid molecule encoding the one or more targets listed in Table 1 and/or Table 2 or fragment thereof, a polypeptide of the one or more targets listed in Table 1 and/or Table 2 or fragment(s) thereof, an activating antibody and/or intrabody that binds to the one or more targets listed in Table 1 and/or Table 2, or a small molecule that binds to the one or more targets listed in Table 1 and/or Table 2. In yet another embodiment, the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target. In another embodiment, the monocytes and/or macrophages are contacted in vitro or ex vivo. In still another embodiment, the monocytes and/or macrophages are primary monocytes and/or primary macrophages. In yet another embodiment, the monocytes and/or macrophages are purified and/or cultured prior to contact with the agent or agents. In another embodiment, the monocytes and/or macrophages are contacted in vivo. In still another embodiment, the monocytes and/or macrophages are contacted in vivo by systemic, peritumoral, or intratumoral administration of the agent. In yet another embodiment, the monocytes and/or macrophages are contacted in a tissue microenvironment.

In another embodiment, the method further comprises contacting the monocytes and/or macrophages with at least one immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus.

In still another aspect, a composition comprising i) a monocyte and/or macrophage generated according to a method described herein and/or ii) an siRNA for downregulating the amount and/or activity of at least one target listed in Table 1 and/or Table 2, is provided.

In yet another aspect, a method of increasing an inflammatory phenotype of monocytes and/or macrophages in a subject after contact with at least one agent comprising administering to the subject an effective amount of the at least one agent, wherein the at least one agent is a) an agent that downregulates the copy number, amount, and/or activity of at least one target listed in Table 1 in or on the monocytes and/or macrophages, and/or b) an agent that upregulates the copy number, amount, and/or activity of at least one target listed in Table 2 in or on the monocytes and/or macrophages, is provided.

As described above, numerous embodiments are further provided that can be applied to any aspect of the present invention and/or combined with any other embodiment described herein. For example, in one embodiment, the monocytes and/or macrophages having the increased inflammatory phenotype exhibit one or more of the following after contact with the agent or agents: a) increased expression and/or secretion of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α); b) decreased expression and/or secretion of CD206, CD163, CD16, CD53, VSIG4, PSGL-1 and/or IL-10; c) increased secretion of at least one cytokine selected from the group consisting of IL-1β, TNF-α, IL-12, IL-18, and IL-23; d) increased ratio of expression of IL-1β, IL-6, and/or TNF-α to expression of IL-10; e) increased CD8+ cytotoxic T cell activation; f) increased CD4+ helper T cell activity; g) increased NK cell activity; h) increased neutrophil activity; i) increased macrophage activity; and/or j) increased spindle-shaped morphology, flatness of appearance, and/or number of dendrites, as assessed by microscopy. In another embodiment, the agent or agents increase the number of Type 1 and/or M1 macrophages, decrease the number of Type 2 and/or M2 macrophages, and/or increase the ratio of i) to ii), wherein i) is Type 1 and/or M1 macrophages and ii) is Type 2 and/or M2 macrophages, in the subject. In still another embodiment, the number and/or activity of cytotoxic CD8+ T cells in the subject is increased after administration of the agent or agents. In yet another embodiment, a method of decreasing an inflammatory phenotype of monocytes and/or macrophages in a subject after contact with at least one agent comprises administering to the subject an effective amount of the at least one agent, wherein the at least one agent is a) an agent that upregulates the copy number, amount, and/or activity of at least one target listed in Table 1 in or on the monocytes and/or macrophages, and/or b) an agent that downregulates the copy number, amount, and/or activity of at least one target listed in Table 2 in or on the monocytes and/or macrophages.

In another embodiment, the monocytes and/or macrophages having the decreased inflammatory phenotype exhibit one or more of the following after contact with the agent or agents: a) decreased expression and/or secretion of cluster of differentiation 80 (CD80), CD86, MHCII, MHCI, interleukin 1-beta (IL-10), IL-6, CCL3, CCL4, CXCL10, CXCL9, GM-CSF and/or tumor necrosis factor alpha (TNF-α); b) increased expression and/or secretion of CD206, CD163, CD16, CD53, VSIG4, PSGL-1 and/or IL-10; c) decreased secretion of at least one cytokine selected from the group consisting of IL-10, TNF-α, IL-12, IL-18, and IL-23; d) decreased ratio of expression of IL-1s, IL-6, and/or TNF-α to expression of IL-10; e) decreased CD8+ cytotoxic T cell activation; f) decreased CD4+ helper T cell activity; g) decreased NK cell activity; h) decreased neutrophil activity; i) decreased macrophage activity; and/or j) decreased spindle-shaped morphology, flatness of appearance, and/or number of dendrites, as assessed by microscopy. In still another embodiment, the agent or agents decrease the number of Type 1 and/or M1 macrophages, increase the number of Type 2 and/or M2 macrophages, and/or decrease the ratio of i) to ii), wherein i) is Type 1 and/or M1 macrophages and ii) is Type 2 and/or M2 macrophages, in the subject. In yet another embodiment, the number and/or activity of cytotoxic CD8+ T cells in the subject is decreased after administration of the agent. In another embodiment, the agent that downregulates the copy number, amount, and/or activity of at least one target listed in Table 1 and/or Table 2 is a small molecule inhibitor, CRISPR guide RNA (gRNA), RNA interfering agent, antisense oligonucleotide, peptide or peptidomimetic inhibitor, aptamer, antibody, intrabody, or cells. The RNA interfering agent may comprise or be, e.g., a small interfering RNA (siRNA), a small hairpin RNA (shRNA), microRNA (miRNA), or a piwi-interacting RNA (piRNA). In still another embodiment, the agent that downregulates the copy number, amount, and/or activity of at least one target listed in Table 1 and/or Table 2 comprises an antibody and/or intrabody, or an antigen binding fragment thereof, which specifically binds to the at least one target listed in Table 1 and/or Table 2. In yet another embodiment, the antibody and/or intrabody, or antigen binding fragment thereof, is camelid, murine, chimeric, humanized, human, detectably labeled, comprises an effector domain, comprises an Fc domain, and/or is selected from the group consisting of Fv, Fav, F(ab′)2, Fab′, dsFv, scFv, sc(Fv)2, and diabodies fragments. In another embodiment, the antibody and/or intrabody, or antigen binding fragment thereof, is conjugated to a cytotoxic agent. In still another embodiment, the cytotoxic agent is selected from the group consisting of a chemotherapeutic agent, a biologic agent, a toxin, and a radioactive isotope. In yet another embodiment, the agent that upregulates the copy number, amount, and/or activity of at least one target listed in Table 1 and/or Table 2 is a nucleic acid molecule encoding the one or more targets listed in Table 1 and/or Table 2 or fragment thereof, a polypeptide of the one or more targets listed in Table 1 and/or Table 2 or fragment(s) thereof, an activating antibody and/or intrabody that binds to the one or more targets listed in Table 1 and/or Table 2, or a small molecule that binds to the one or more targets listed in Table 1 and/or Table 2. In another embodiment, the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target. In still another embodiment, the agent or agents are administered in vivo by systemic, peritumoral, or intratumoral administration of the agent. In yet another embodiment, the agent or agents contact the monocytes and/or macrophages in a tissue microenvironment. In another embodiment, the method further comprises contacting the monocytes and/or macrophages with at least one immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus.

In another aspect, a method of increasing inflammation in a subject comprising administering to the subject an effective amount of a) monocytes and/or macrophages contacted with at least one agent to downregulate the copy number, amount, and/or activity of at least one target listed in Table 1 and/or b) monocytes and/or macrophages contacted with at least one agent to upregulate the copy number, amount, and/or activity of at least one target listed in Table 2, is provided.

As described above, numerous embodiments are further provided that can be applied to any aspect of the present invention and/or combined with any other embodiment described herein. For example, in one embodiment, the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target. In another embodiment, the monocytes and/or macrophages are genetically engineered, autologous, syngeneic, or allogeneic relative to the subject's monocytes and/or macrophages. In still another embodiment, the monocytes and/or macrophages contacted with the at least one agent of a) are different from the monocytes and/or macrophages contacted with the at least one agent of b). In yet another embodiment, the monocytes and/or macrophages contacted with the at least one agent of a) are the same as the monocytes and/or macrophages contacted with the at least one agent of b). In another embodiment, the agent or agents are administered systemically, peritumorally, or intratumorally.

In still another aspect, a method of decreasing inflammation in a subject comprising administering to the subject an effective amount of a) monocytes and/or macrophages contacted with at least one agent to upregulate the copy number, amount, and/or activity of at least one target listed in Table 1 and/or b) monocytes and/or macrophages contacted with at least one agent to downregulate the copy number, amount, and/or activity of at least one target listed in Table 2, is provided.

As described above, numerous embodiments are further provided that can be applied to any aspect of the present invention and/or combined with any other embodiment described herein. For example, in one embodiment, the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target. In another embodiment, the monocytes and/or macrophages are genetically engineered, autologous, syngeneic, or allogeneic relative to the subject's monocytes and/or macrophages. In still another embodiment, the monocytes and/or macrophages contacted with the at least one agent of a) are different from the monocytes and/or macrophages contacted with the at least one agent of b). In yet another embodiment, the monocytes and/or macrophages contacted with the at least one agent of a) are the same as the monocytes and/or macrophages contacted with the at least one agent of b). In another embodiment, the agent or agents are administered systemically, peritumorally, or intratumorally.

In yet another aspect, a method of sensitizing cancer cells in a subject to cytotoxic CD8+ T cell-mediated killing and/or immune checkpoint therapy comprising administering to the subject a therapeutically effective amount of a) at least one agent that downregulates the copy number, amount, and/or activity of at least one target listed in Table 1 in or on monocytes and/or macrophages and/or b) at least one agent that upregulates the copy number, amount, and/or activity of at least one target listed in Table 2 in or on monocytes and/or macrophage, is provided.

As described above, numerous embodiments are further provided that can be applied to any aspect of the present invention and/or combined with any other embodiment described herein. For example, in one embodiment, the method further comprises administering at least one agent that downregulates the copy number, amount, and/or activity of at least one target listed in Table 1. In another embodiment, the agent is a small molecule inhibitor, CRISPR guide RNA (gRNA), RNA interfering agent, antisense oligonucleotide, peptide or peptidomimetic inhibitor, aptamer, antibody, intrabody, or cells. The RNA interfering agent may comprise or be, e.g., a small interfering RNA (siRNA), a small hairpin RNA (shRNA), microRNA (miRNA), or a piwi-interacting RNA (piRNA). In still another embodiment, the agent comprises an antibody and/or intrabody, or an antigen binding fragment thereof, which specifically binds to the at least one target listed in Table 1. In yet another embodiment, the antibody and/or intrabody, or antigen binding fragment thereof, is camelid, murine, chimeric, humanized, human, detectably labeled, comprises an effector domain, comprises an Fc domain, and/or is selected from the group consisting of Fv, Fav, F(ab′)2, Fab′, dsFv, scFv, sc(Fv)2, and diabodies fragments. In another embodiment, the antibody and/or intrabody, or antigen binding fragment thereof, is conjugated to a cytotoxic agent. In still another embodiment, the cytotoxic agent is selected from the group consisting of a chemotherapeutic agent, a biologic agent, a toxin, and a radioactive isotope. In yet another embodiment, the method further comprises administering at least one agent that upregulates the copy number, amount, and/or activity of at least one target listed in Table 2. In another embodiment, the agent is a nucleic acid molecule encoding the one or more targets listed in Table 2 or fragment thereof, a polypeptide of the one or more targets listed in Table 2 or fragment(s) thereof, an activating antibody and/or intrabody that binds to the one or more targets listed in Table 2, or a small molecule that binds to the one or more targets listed in Table 2.

In another aspect, a method of sensitizing cancer cells in a subject afflicted with a cancer to cytotoxic CD8+ T cell-mediated killing and/or immune checkpoint therapy comprising administering to the subject a therapeutically effective amount of a) monocyte cells and/or macrophage cells contacted with at least one agent to downregulate the copy number, amount, and/or activity of at least one target listed in Table 1 and/or b) monocyte cells and/or macrophage cells contacted with at least one agent to upregulate the copy number, amount, and/or activity of at least one target listed in Table 2, is provided.

As described above, numerous embodiments are further provided that can be applied to any aspect of the present invention and/or combined with any other embodiment described herein. For example, in one embodiment, the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target. In another embodiment, the monocytes and/or macrophages are genetically engineered, autologous, syngeneic, or allogeneic relative to the subject's monocytes and/or macrophages. In still another embodiment, the monocytes and/or macrophages contacted with the at least one agent of a) are different from the monocytes and/or macrophages contacted with the at least one agent of b). In yet another embodiment, the monocytes and/or macrophages contacted with the at least one agent of a) are the same as the monocytes and/or macrophages contacted with the at least one agent of b). In another embodiment, the agent or agents are administered systemically, peritumorally, or intratumorally. In still another embodiment, the method further comprises treating the cancer in the subject by administering to the subject at least one immunotherapy, optionally wherein the immunotherapy comprises an immune checkpoint inhibitor, immune-stimulatory agonist, inflammatory agent, cells, a cancer vaccine, and/or a virus. In yet another embodiment, the immune checkpoint is selected from the group consisting of PD-1, PD-L1, PD-L2, and CTLA-4. In another embodiment, the immune checkpoint is PD-1. In still another embodiment, the agent or agents reduce the number of proliferating cells in the cancer and/or reduce the volume or size of a tumor comprising the cancer cells. In yet another embodiment, the agent or agents increase the amount and/or activity of CD8+ T cells infiltrating a tumor comprising the cancer cells. In still another embodiment, the agent or agents a) increase the amount and/or activity of M1 macrophages infiltrating a tumor comprising the cancer cells and/or b) decrease the amount and/or activity of M2 macrophages infiltrating a tumor comprising the cancer cells. In yet another embodiment, the method further comprises administering to the subject at least one additional therapy or regimen for treating the cancer. In another embodiment, the therapy is administered before, concurrently with, or after the agent.

In still another aspect, a method of identifying monocytes and/or macrophages that can increase an inflammatory phenotype thereof by modulating at least one target comprising: a) determining the copy number, amount, and/or activity of at least one target listed in Table 1 and/or Table 2 from the monocytes and/or macrophages; b) determining the copy number, amount, and/or activity of the at least one target in a control; and c) comparing the copy number, amount, and/or activity of the at least one target detected in steps a) and b); wherein the presence of, or an increase in, the copy number, amount, and/or activity of, the at least one target listed in Table 1 and/or the absence of, or a decrease in, the copy number, amount, and/or activity of, the at least one target listed in Table 2, in the monocytes and/or macrophages relative to the control copy number, amount, and/or activity of the at least one target indicates that the monocytes and/or macrophages can increase the inflammatory phenotype thereof by modulating the at least one target, is provided.

As described above, numerous embodiments are further provided that can be applied to any aspect of the present invention and/or combined with any other embodiment described herein. For example, in one embodiment, the method further comprises contacting the cells with, recommending, prescribing, or administering an agent that modulates the at least one target listed in Table 1 and/or Table 2. In another embodiment, the method further comprises contacting the cells with, recommending, prescribing, or administering cancer therapy other than an agent that modulates the one or more targets listed in Table 1 and/or Table 2 if the subject is determined not to benefit from increasing an inflammatory phenotype by modulating the one or more targets. In still another embodiment, the method further comprises contacting the cells with and/or administering at least one additional agent that increases an immune response. In yet another embodiment, the additional agent is selected from the group consisting of targeted therapy, chemotherapy, radiation therapy, and/or hormonal therapy. In another embodiment, the control is from a member of the same species to which the subject belongs. In still another embodiment, the control is a sample comprising cells. In yet another embodiment, the subject is afflicted with a cancer. In another embodiment, the control is a cancer sample from the subject. In still another embodiment, the control is a non-cancer sample from the subject.

In yet another aspect, a method of identifying monocytes and/or macrophages that can decrease an inflammatory phenotype thereof by modulating at least one target comprising: a) determining the copy number, amount, and/or activity of at least one target listed in Table 1 and/or Table 2 from the monocytes and/or macrophages; b) determining the copy number, amount, and/or activity of the at least one target in a control; and c) comparing the copy number, amount, and/or activity of the at least one target detected in steps a) and b); wherein the absence of, or a decrease in, the copy number, amount, and/or activity of, the at least one target listed in Table 1 and/or the presence of, or an increase in, the copy number, amount, and/or activity of, the at least one target listed in Table 2, in the monocytes and/or macrophages relative to the control copy number, amount, and/or activity of the at least one target indicates that the monocytes and/or macrophages that can decrease the inflammatory phenotype thereof by modulating the at least one target, is provided.

As described above, numerous embodiments are further provided that can be applied to any aspect of the present invention and/or combined with any other embodiment described herein. For example, in one embodiment, the method further comprises contacting the monocytes and/or macrophages with, recommending, prescribing, or administering an agent or agents that modulate the one or more targets listed in Table 1 and/or Table 2. In another embodiment, the method further comprises contacting the monocytes and/or macrophages with, recommending, prescribing, or administering cancer therapy other than an agent or agents that modulate the one or more targets listed in Table 1 and/or Table 2 if the subject is determined not to benefit from decreasing an inflammatory phenotype by modulating the at least one target. In still another embodiment, the method further comprises contacting the monocytes and/or macrophages with and/or administering at least one additional agent that decreases an immune response. In yet another embodiment, the control is from a member of the same species to which the subject belongs. In another embodiment, the control is a sample comprising cells. In still another embodiment, the subject is afflicted with a cancer. In another embodiment, the control is a cancer sample from the subject. In still another embodiment, the control is a non-cancer sample from the subject.

In another aspect, a method for predicting the clinical outcome of a subject afflicted with a cancer, the method comprising: a) determining the copy number, amount, and/or activity of at least one target listed in Table 1 and/or Table 2 from monocytes and/or macrophages from the subject; b) determining the copy number, amount, and/or activity of the at least one target from a control having a poor clinical outcome; and c) comparing the copy number, amount, and/or activity of the at least one target in the subject sample and in the sample from the control subject; wherein the presence of, or an increase in, the copy number, amount, and/or activity of, the at least one target listed in Table 1 and/or the absence of, or a decrease in, the copy number, amount, and/or activity of, the at least one target listed in Table 2, from the monocytes and/or macrophages from the subject as compared to the copy number, amount and/or activity in the control, indicates that the subject does not have a poor clinical outcome, is provided.

In still another aspect, a method for monitoring the inflammatory phenotype of monocytes and/or macrophages in a subject is provided, the method comprising: a) detecting in a first subject sample at a first point in time the copy number, amount, and/or or activity of at least one target listed in Table 1 and/or Table 2 from monocytes and/or macrophages from the subject; b) repeating step a) using a subsequent sample comprising monocytes and/or macrophages obtained at a subsequent point in time; and c) comparing the amount or activity of at least one target listed in Table 1 and/or Table 2 detected in steps a) and b), wherein the absence of, or a decrease in, the copy number, amount, and/or activity of, the at least one target listed in Table 1 and/or the presence of, or an increase in, the copy number, amount, and/or activity of, the at least one target listed in Table 2, from the monocytes and/or macrophages from the subsequent sample as compared to the copy number, amount and/or activity from the monocytes and/or macrophages from the first sample indicates that the subject's monocytes and/or macrophages have an upregulated inflammatory phenotype; or wherein the presence of, or an increase in, the copy number, amount, and/or activity of, the at least one target listed in Table 1 and/or the absence of, or a decrease in, the copy number, amount, and/or activity of, the at least one target listed in Table 2, from the monocytes and/or macrophages from the subsequent sample as compared to the copy number, amount and/or activity from the monocytes and/or macrophages from the first samples indicates that the subject's monocytes and/or macrophages have a downregulated inflammatory phenotype.

As described above, numerous embodiments are further provided that can be applied to any aspect of the present invention and/or combined with any other embodiment described herein. For example, in one embodiment, the first and/or at least one subsequent sample comprises monocytes and/or macrophages that are cultured in vitro. In another embodiment, the first and/or at least one subsequent sample comprises monocytes and/or macrophages that are not cultured in vitro. In still another embodiment, the first and/or at least one subsequent sample is a portion of a single sample or pooled samples obtained from the subject. In yet another embodiment, the sample comprises blood, serum, peritumoral tissue, and/or intratumoral tissue obtained from the subject.

In yet another aspect, a method of assessing the efficacy of an agent for increasing an inflammatory phenotype of monocytes and/or macrophages in a subject, comprising: a) detecting in a subject sample comprising monocytes and/or macrophages at a first point in time i) the copy number, amount, and/or or activity of at least one target listed in Table 1 and/or Table 2 in or on the monocytes and/or macrophages and/or ii) an inflammatory phenotype of the monocytes and/or macrophages; b) repeating step a) during at least one subsequent point in time after the monocytes and/or macrophages are contacted with the agent; and c) comparing the value of i) and/or ii) detected in steps a) and b), wherein the absence of, or a decrease in, the copy number, amount, and/or activity of, the at least one target listed in Table 1 and/or the presence of, or an increase in, the copy number, amount, and/or activity of, the at least one target listed in Table 2, and/or an increase in ii) in the subsequent sample as compared to the copy number, amount, and/or activity in the sample at the first point in time, indicates that the agent increases the inflammatory phenotype of monocytes and/or macrophages in the subject, is provided.

As described above, numerous embodiments are further provided that can be applied to any aspect of the present invention and/or combined with any other embodiment described herein. For example, in one embodiment, the monocytes and/or macrophages contacted with the agent are comprised within a population of cells and the agent increases the number of Type 1 and/or M1 macrophages in the population of cells. In another embodiment, the monocytes and/or macrophages contacted with the agent are comprised within a population of cells and the agent decreases the number of Type 2 and/or M2 macrophages in the population of cells.

In another aspect, a method of assessing the efficacy of an agent for decreasing an inflammatory phenotype of monocytes and/or macrophages, comprising: a) detecting in a subject sample comprising monocytes and/or macrophages at a first point in time i) the copy number, amount, and/or or activity of at least one target listed in Table 1 and/or Table 2 in or on the monocytes and/or macrophages and/or ii) an inflammatory phenotype of the monocytes and/or macrophages; b) repeating step a) during at least one subsequent point in time after the monocytes and/or macrophages are contacted with the agent; and c) comparing the value of i) and/or ii) detected in steps a) and b), wherein the presence of, or an increase in, the copy number, amount, and/or activity of, the at least one target listed in Table 1 and/or the absence of, or a decrease in, the copy number, amount, and/or activity of, the at least one target listed in Table 2, and/or a decrease in ii) in the subsequent sample as compared to the copy number, amount, and/or activity in the sample at the first point in time, indicates that the agent decreases the inflammatory phenotype of monocytes and/or macrophages in the subject.

As described above, numerous embodiments are further provided that can be applied to any aspect of the present invention and/or combined with any other embodiment described herein. For example, in one embodiment, the monocytes and/or macrophages contacted with the agent are comprised within a population of cells and the agent selectively decreases the number of Type 1 and/or M1 macrophages in the population of cells. In another embodiment, the monocytes and/or macrophages contacted with the agent are comprised within a population of cells and the agent selectively increases the number of Type 2 and/or M2 macrophages in the population of cells. In still another embodiment, the monocytes and/or macrophages are contacted in vitro or ex vivo. In yet another embodiment, the monocytes and/or macrophages are primary monocytes and/or primary macrophages. In another embodiment, the monocytes and/or macrophages are purified and/or cultured prior to contact with the agent. In still another embodiment, the monocytes and/or macrophages are contacted in vivo. In yet another embodiment, the monocytes and/or macrophages are contacted in vivo by systemic, peritumoral, or intratumoral administration of the agent. In another embodiment, the monocytes and/or macrophages are contacted in a tissue microenvironment. In still another embodiment, the method described herein further comprises contacting the monocytes and/or macrophages with at least one immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus. In yet another embodiment, the subject is a mammal. In another embodiment, the mammal is a non-human animal model or a human.

In still another aspect, a method of assessing the efficacy of an agent for treating a cancer in a subject, comprising: a) detecting in a subject sample comprising monocytes and/or macrophages at a first point in time i) the copy number, amount, and/or or activity of at least one target listed in Table 1 and/or Table 2 in or on monocytes and/or macrophages and/or ii) an inflammatory phenotype of the monocytes and/or macrophages; b) repeating step a) during at least one subsequent point in time after administration of the agent; and c) comparing the value of i) and/or ii) detected in steps a) and b), wherein the absence of, or a decrease in, the copy number, amount, and/or activity of, the at least one target listed in Table 1 and/or the presence of, or an increase in, the copy number, amount, and/or activity of, the at least one target listed in Table 2, and/or an increase in ii) in or on the monocytes and/or macrophages of the subject sample at the subsequent point in time as compared to the copy number, amount, and/or activity in or on the monocytes and/or macrophages of the subject sample at the first point in time, indicates that the agent treats the cancer in the subject, is provided.

As described above, numerous embodiments are further provided that can be applied to any aspect of the present invention and/or combined with any other embodiment described herein. For example, in one embodiment, between the first point in time and the subsequent point in time, the subject has undergone treatment, completed treatment, and/or is in remission for the cancer. In another embodiment, the first and/or at least one subsequent sample is selected from the group consisting of ex vivo and in vivo samples. In still another embodiment, the first and/or at least one subsequent sample is obtained from a non-human animal model of the cancer.

In yet another embodiment, the first and/or at least one subsequent sample is a portion of a single sample or pooled samples obtained from the subject. In another embodiment, the sample comprises cells, serum, peritumoral tissue, and/or intratumoral tissue obtained from the subject.

In yet another aspect, a method for screening for agents that sensitize cancer cells to cytotoxic T cell-mediated killing and/or immune checkpoint therapy comprising a) contacting cancer cells with cytotoxic T cells and/or immune checkpoint therapy in the presence of monocytes and/or macrophages contacted with i) at least one agent that decreases the copy number, amount, and/or activity of at least one target listed in Table 1 and/or ii) at least one agent that increases the copy number, amount, and/or activity of the at least one target listed in Table 2; b) contacting cancer cells with cytotoxic T cells and/or immune checkpoint therapy in the presence of control monocytes and/or macrophages that are not contacted with the at least one agent or agents; and c) identifying agents that sensitize cancer cells to cytotoxic T cell-mediated killing and/or immune checkpoint therapy by identifying agents that increase cytotoxic T cell-mediated killing and/or immune checkpoint therapy efficacy in a) compared to b), is provided.

In another aspect, a method for screening for agents that sensitize cancer cells to cytotoxic T cell-mediated killing and/or immune checkpoint therapy comprising a) contacting cancer cells with cytotoxic T cells and/or immune checkpoint therapy in the presence of monocytes and/or macrophages engineered to decrease the copy number, amount, and/or activity of at least one target listed in Table 1 and/or ii) engineered to increase the copy number, amount, and/or activity of the at least one target listed in Table 2; b) contacting cancer cells with cytotoxic T cells and/or immune checkpoint therapy in the presence of control monocytes and/or macrophages; and c) identifying agents that sensitize cancer cells to cytotoxic T cell-mediated killing and/or immune checkpoint therapy by identifying agents that increase cytotoxic T cell-mediated killing and/or immune checkpoint therapy efficacy in a) compared to b), is provided.

As described above, numerous embodiments are further provided that can be applied to any aspect of the present invention and/or combined with any other embodiment described herein. For example, in one embodiment, the step of contacting occurs in vivo, ex vivo, or in vitro. In another embodiment, the method further comprises determining a reduction in i) the number of proliferating cells in the cancer and/or ii) a reduction in the volume or size of a tumor comprising the cancer cells. In still another embodiment, the method further comprises determining i) an increased number of CD8+ T cells and/or ii) an increased number of Type 1 and/or M1 macrophages infiltrating a tumor comprising the cancer cells. In yet another embodiment, the method further comprises determining responsiveness to the agent that modulates the at least one target listed in Table 1 and/or Table 2 measured by at least one criterion selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria. In another embodiment, the method further comprises contacting the cancer cells with at least one additional cancer therapeutic agent or regimen. In still another embodiment, the agent or agents further comprise a lipid or lipidoid. In yet another embodiment, the lipidoid is of Formula (VI):

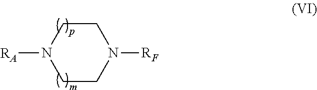

wherein: p is an integer between 1 and 3, inclusive; m is an integer between 1 and 3, inclusive; R_{A} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

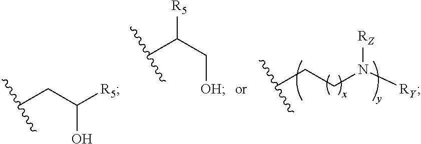

R_{F} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

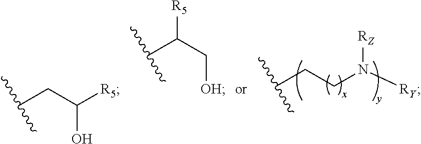

each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;
wherein, at least one of R_{A}, R_{F}, R_{Y}, and R_{Z} is

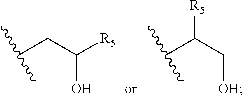

each occurrence of x is an integer between 1 and 10, inclusive; each occurrence of y is an integer between 1 and 10, inclusive; each occurrence of R_{Y} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl; OH or

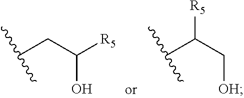

each occurrence of R_{Z} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl: substituted or unsubstituted heteroaryl,

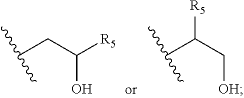

or a pharmaceutically acceptable salt thereof. In another embodiment, p is 1. In still another embodiment, m is 1. In yet another embodiment, each of p and m are 1. In yet another embodiment, R_{F} is

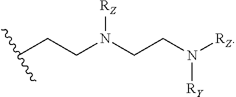

In another embodiment, R_{A} is

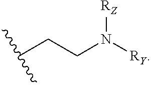

In still another embodiment, the compound of Formula (VI) is of the formula:

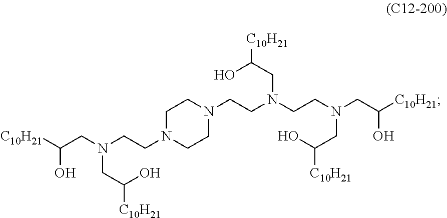

or a salt thereof. In yet another embodiment, the composition is in the form a lipid nanoparticle. In another embodiment, the lipid nanoparticle comprises about 1.0% to about 60.0% by mole of C12-200. In still another embodiment, the lipid nanoparticle further comprises one or more co-lipids. In yet another embodiment, each co-lipid is selected from disteroylphosphatidyl choline (DSPC), cholesterol, and DMG-PEG. In another embodiment, the concentration of DSPC is about 1.0% to about 20.0% by mole. In still another embodiment, the concentration of cholesterol is about 10.0% to about 50.0% by mole. In yet another embodiment, the concentration of DMG-PEG is about 0.1% to about 5.0% by mole. In another embodiment, DSPC is present a concentration of about 1.0% to about 20.0% by mole; cholesterol is present at a concentration of about 10.0% to about 50.0% by mole; and DMG-PEG is present a concentration of about 0.1% to about 5.0% by mole. In still another embodiment, the agent is in a pharmaceutically acceptable formulation. In yet another embodiment, the monocytes and/or macrophages having a modulated inflammatory phenotype exhibit one or more of the following: a) modulated expression of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α); b) modulated expression of CD206, CD163, CD16, CD53, VSIG4, PSGL-1 and/or IL-10; c) modulated secretion of at least one cytokine selected from the group consisting of IL-0, TNF-α, IL-12, IL-18, and IL-23; d) modulated ratio of expression of IL-1β, IL-6, and/or TNF-α to expression of IL-10; e) modulated CD8+ cytotoxic T cell activation; f) modulated CD4+ helper T cell activity; g) modulated NK cell activity; h) modulated neutrophil activity; i) modulated macrophage activity; and/or j) modulated spindle-shaped morphology, flatness of appearance, and/or dendrite numbers, as assessed by microscopy. In another embodiment, the cells and/or macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express or are determined to express at least one target selected from the group consisting of targets listed in Table 1 and/or Table 2. In still another embodiment, the at least one target listed in Table 1 is selected from the group consisting of human SIGLEC9, VSIG4, CD74, CD207, LRRC25, SELPLG, AIF1, CD84, IGSF6, CD48, CD33, LST1, TNFAIP8L2 (TIPE2), SPI1 (PU.1), LLRB2, CCR5, EVI2B, CLEC7A, TBXAS1, SIGLEC7, and DOCK2, or a fragment thereof. In yet another embodiment, the at least one target listed in Table 2 is selected from the group consisting of human CD53, FERMT3, CD37, CXorf21, CD48, and CD84, or a fragment thereof. In another embodiment, the cancer is a solid tumor that is infiltrated with macrophages, wherein the infiltrating macrophages represent at least about 5% of the mass, volume, and/or number of cells in the tumor or the tumor microenvironment, and/or wherein the cancer is selected from the group consisting of mesothelioma, kidney renal clear cell carcinoma, glioblastoma, lung adenocarcinoma, lung squamous cell carcinoma, pancreatic adenocarcinoma, breast invasive carcinoma, acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous, cystadenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, and uveal melanoma. In still another embodiment, the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the macrophages are TAMs and/or M2 macrophages. In yet another embodiment, the macrophages express or are determined to express one or more targets selected from the group consisting of targets listed in Table 1 and/or Table 2. In another embodiment, the at least one target listed in Table 1 is selected from the group consisting of human SIGLEC9, VSIG4, CD74, CD207, LRRC25, SELPLG, AIF1, CD84, IGSF6, CD48, CD33, LST1, TNFAIP8L2 (TIPE2), SPI1 (PU.1), LILRB2, CCR5, EVI2B, CLEC7A, TBXAS1, SIGLEC7, and DOCK2, or a fragment thereof. In still another embodiment, the at least one target listed in Table 2 is selected from the group consisting of human CD53, FERMT3, CD37, CXorf21, CD48, and CD84, or a fragment thereof. In yet another embodiment, the monocytes and/or macrophages are primary monocytes and/or primary macrophages. In another embodiment, the monocytes and/or macrophages are comprised within a tissue microenvironment. In still another embodiment, the monocytes and/or macrophages are comprised within a human tumor model or an animal model of cancer. In yet another embodiment, the subject is a mammal. In another embodiment, the mammal is a human. In still another embodiment, the human is afflicted with a cancer.

### BRIEF DESCRIPTION OF THE DRAWINGS

FIG. 1A-FIG. 1C show phenotype and morphology of macrophages driven to different differentiation states. FIG. 1A shows the expression of classical M2 biomarkers after macrophage differentiation. FIG. 1B shows the expression of new M2 biomarkers after macrophage differentiation. FIG. 1C shows morphological images of M1- and M2c-differentiated macrophages.

FIG. 2A-FIG. 2Y show IC50 curves for siRNAs directed against individual macrophage-associated targets.

FIG. 3A-FIG. 3E show characterization of surface phenotype and morphology after knockdown of macrophage-associated targets in primary human macrophages. These figures show the effects of siRNA-mediated targe knockdown on target mRNA knockdown (FIG. 3A), cell surface expression of targets (FIG. 3B), classical macrophage phenotypic markers (FIG. 3C), new macrophage phenotypic markers (FIG. 3D), and macrophage morphology (FIG. 3E).

FIG. 4A-FIG. 4G show characterization of modulated macrophage phenotype and function after inhibition of macrophage-associated targets in primary human macrophages. These figures show the effects of antibody-mediated target inhibition on decreasing classical M2 markers in the presence of M2-skewing conditions (FIG. 4A); new M2 markers in the presence of M2-skewing conditions(FIG. 4B); increasing M1 pro-inflammatory cytokines in the presence of M2-skewing conditions (FIG. 4C); decreasing classical M2 markers, in a dose-dependent fashion, when added after M2-skewing conditions (FIG. 4D); decreasing new M2 markers, in a dose-dependent fashion, when added after M2-skewing conditions (FIG. 4E); and increasing M1 pro-inflammatory cytokine production when added after M2-skewing conditions (FIGS. 4F and 4G).

FIG. 5A-FIG. 5C show the results of Staphylococcal enterotoxin B (SEB) assay experiments. FIG. 5A shows the results of intracellular cytokine staining of CD3+ T cells after 4 days. FIGS. 5B and 5C show the results of cytokine production after 4 days.

FIG. 6A-FIG. 6B show the results of one-way mixed lymphocyte reaction (MLR) assay experiments. FIG. 6A shows the results of intracellular staining of CD8+ T cells. Data are shown as the fold-change over isotype control. FIG. 6B shows the results of cytokine production. Data are shown as the fold-change over isotype control.

FIG. 7A-FIG. 7B show the results of flow cytometry analyses of macrophage-associated target expression on tumor associated macrophages (TAMs) from a variety of cancer types.

FIG. 8A-FIG. 8D show cytokine production from dissociated tumor samples and tumor slice samples representing 6 different tumor types treated with individual or combinations of antibodies.

FIG. 9A-FIG. 9C show results of tumor slice cultures. Data are shown as the fold-change over isotype background as controls for lung tumor slice (FIG. 9A), GI tumor slice (FIG. 9B), and kidney tumor slice (FIG. 9C) cultures.

FIG. 10A-FIG. 10C shows the results of an analysis of immune compositions within tumors. Data are shown for GI tumor (FIG. 10A), kidney tumor (FIG. 10B), and the CD45+ and CD3+ compositions of antibody-treated tumor slices (FIG. 10C).

FIG. 11 shows the percentage of tumors containing a macrophage (CD11b) signature indicating infiltration of TAMs.

For any figure showing a bar histogram, curve, or other data associated with a legend, the bars, curve, or other data presented from left to right for each indication correspond directly and in order to the boxes from top to bottom of the legend.

### DETAILED DESCRIPTION OF THE INVENTION

It has been determined herein that certain targets regulate monocyte and/or macrophage inflammatory phenotype, polarization, activation, and/or function. Accordingly, the present invention relates, in part, to methods of modulating the copy number, amount, and/or activity of one or more biomarkers described herein (e.g., targets listed in Table 1, Table 2, Examples, etc.). and uses of the biomarkers and/or modulatory agents thereof for treating, diagnosing, prognosing, and screening purposes as described further below.

### I. Definitions

The term “about,” in some embodiments, encompasses values that are within 1%, 2%, 3%, 4%, 5%, 6%, 7%, 8%, 9%, or 10%, inclusive, or any range in between (e.g., plus or minus 2%-6%), of a value that is measured. In some embodiments, the term “about” refers to the inherent variation of error in a method, assay, or measured value, such as the variation that exists among experiments.

The term “activating receptor” includes immune cell receptors that bind antigen, complexed antigen (e.g., in the context of major histocompatibility complex (MHC) polypeptides), or bind to antibodies. Such activating receptors include T cell receptors (TCR), B cell receptors (BCR), cytokine receptors, LPS receptors, complement receptors, Fc receptors, and other ITAM containing receptors. For example, T cell receptors are present on T cells and are associated with CD3 polypeptides. T cell receptors are stimulated by antigen in the context of MHC polypeptides (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes. Similar to T cells activation of macrophages via activation receptors such as, cytokine receptors or pattern associated molecular pattern (PAMP) receptors, results in changes such as protein phosphorylation, alteration to surface receptor phenotype, protein synthesis and release, as well as morphologic changes.

The term “administering” relates to the actual physical introduction of an agent into or onto (as appropriate) a biological target of interest, such as a host and/or subject. A composition can be administered to the cell (e.g., “contacting”) in vitro or in vivo. A composition can be administered to the subject in vivo via an appropriate route of administration. Any and all methods of introducing the composition into the host are contemplated according to the present invention. The method is not dependent on any particular means of introduction and is not to be so construed. Means of introduction are well-known to those skilled in the art, and are also exemplified herein. The term include routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which can detrimentally affect its ability to perform its intended function. The agent can be administered alone, or in conjunction with a pharmaceutically acceptable carrier. The agent also can be administered as a prodrug, which is converted to its active form in vivo.

The term “agent” refers to a compound, supramolecular complex, material, and/or combination or mixture thereof. A compound (e.g., a molecule) can be represented by a chemical formula, chemical structure, or sequence. Representative, non-limiting examples of agents, include, e.g., small molecules, polypeptides, proteins, polynucleotides (e.g., RNAi agents, siRNA, miRNA, piRNA, mRNA, antisense polynucleotides, aptamers, and the like), lipids, and polysaccharides. In general, agents can be obtained using any suitable method known in the art. In some embodiments, an agent can be a “therapeutic agent” for use in treating a disease or disorder (e.g., cancer) in a subject (e.g., a human).

The term “agonist” refers to an agent that binds to a target(s) (e.g., a receptor) and activates or increases the biological activity of the target(s). For example, an “agonist” antibody is an antibody that activates or increases the biological activity of the antigen(s) it binds.

The term “altered amount” or “altered level” encompasses increased or decreased copy number (e.g., germline and/or somatic) of a biomarker nucleic acid, or increased or decreased expression level in a sample of interest, as compared to the copy number or expression level in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein can be determined by detecting posttranslational modification such as methylation status of the marker, which can affect the expression or activity of the biomarker protein. In some embodiments, the “altered amount” refers to the presence or absence of a biomarker because the reference baseline may be the absence or presence of the biomarker, respectively. The absence or presence of the biomarker can be determined according to the threshold of sensitivity of a given assay used to measure the biomarker.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000%, or than that amount. Alternatively, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker). The term “expression” encompasses the processes by which nucleic acids (e.g., DNA) are transcribed to produce RNA, and can also refer to the processes by which RNA transcripts are processed and translated into polypeptides. The sum of expression of nucleic acids and their polypeptide counterparts, if any, contributes to the amount of a biomarker, such as one or more targets listed in Table 1 and/or Table 2.

The term “altered activity” of a biomarker refers to an activity of the biomarker which is increased or decreased in a disease state, e.g., in a cancer sample, or a treated state, as compared to the activity of the biomarker in a normal, control sample. Altered activity of the biomarker can be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations can be present in the coding or non-coding region of the biomarker nucleic acid.

The term “altered subcellular localization” of a biomarker refers to the mislocalization of the biomarker within a cell relative to the normal localization within the cell e.g., within a healthy and/or wild-type cell. An indication of normal localization of the marker can be determined through an analysis of subcellular localization motifs known in the field that are harbored by biomarker polypeptides.

The term “antagonist” or “blocking” refers to an agent that binds to a target(s) (e.g., a receptor) and inhibits or reduces the biological activity of the target(s). For example, an “antagonist” antibody is an antibody that significantly inhibits or reduces biological activity of the antigen(s) it binds.

Unless otherwise specified here within, the terms “antibody” and “antibodies” broadly encompass naturally-occurring forms of antibodies (e.g., IgG, IgA, IgM, IgE) and recombinant antibodies, such as single-chain antibodies, chimeric and humanized antibodies and multi-specific antibodies, as well as fragments, fusion proteins, and derivatives of all of the foregoing, which fragments and derivatives have at least an antigenic binding site. Antibody derivatives can comprise a protein or chemical moiety conjugated to an antibody.

In addition, “intrabodies” are a type of well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publ. Numbers WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “biomarker” refers to a gene or gene product that is a target for modulating one or more phenotypes of interest, such as a phenotype of interest in monocytes and/or macrophages. In this context, the term “biomarker” is synonymous with “target.” In some embodiments, however, the term further encompasses a measurable entity of the target that has been determined to be indicative of an output of interest, such as one or more diagnostic, prognostic, and/or therapeutic outputs (e.g., for modulating an inflammatory phenotype, cancer state, and the like). Biomarkers can include, without limitation, nucleic acids (e.g., genomic nucleic acids and/or transcribed nucleic acids) and proteins, particularly those listed in Table 1 and Table 2. In one embodiment, such targets are negative regulators of inflammatory phenotype, immune response, and/or T cell-mediated cytotoxicity shown in Table 1 and/or positive regulators of inflammatory phenotype, immune response, and/or T cell-mediated cytotoxicity shown in Table 2.

The terms “cancer” or “tumor” or “hyperproliferative” refer to the presence of cells possessing characteristics typical of cancer-causing cells, such as uncontrolled proliferation, immortality, invasive or metastatic potential, rapid growth, and certain characteristic morphological features. In some embodiments, such cells exhibit such characteristics in part or in full due to the expression and activity of immune checkpoint proteins, such as PD-1, PD-L1, PD-L2, and/or CTLA-4.

Cancer cells are often in the form of a tumor, but such cells can exist alone within an animal, or can be a non-tumorigenic cancer cell, such as a leukemia cell. As used herein, the term “cancer” includes premalignant as well as malignant cancers. Cancers include, but are not limited to, a variety of cancers, carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood, malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bone cancer, brain tumor, lung carcinoma (including lung adenocarcinoma), small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In some embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, Brenner, or undifferentiated. In some embodiments, the cancer is selected from the group consisting of (advanced) non-small cell lung cancer, melanoma, head and neck squamous cell cancer, (advanced) urothelial bladder cancer, (advanced) kidney cancer (RCC), microsatellite instability-high cancer, classical Hodgkin lymphoma, (advanced) gastric cancer, (advanced) cervical cancer, primary mediastinal B-cell lymphoma, (advanced) hepatocellular carcinoma, and (advanced) merkel cell carcinoma.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 60%, 65%, 70%, 75%, 80%, 85%, 90%, 95%, 98%, 99%, 99.5%, 99.9%, or greater of the nucleotide residues of the first portion are capable of base pairing with nucleotide residues in the second portion. More preferably, all nucleotide residues of the first portion are capable of base pairing with nucleotide residues in the second portion. In some embodiments, complementary polynucleotides can be “sufficiently complementary” or can have “sufficient complementarity,” that is, complementarity sufficient to maintain a duplex and/or have a desired activity. For example, in the case of RNAi agents, such complementarity is complementarity between the agent and a target mRNA that is sufficient to partly or completely prevent translation of the mRNA. For example, an siRNA having a “sequence sufficiently complementary to a target mRNA sequence to direct target-specific RNA interference (RNAi)” means that the siRNA has a sequence sufficient to trigger the destruction of the target mRNA by the RNAi machinery or process.

The term “substantially complementary” refers to complementarity in a base-paired, double-stranded region between two nucleic acids and not any single-stranded region such as a terminal overhang or a gap region between two double-stranded regions. The complementarity does not need to be perfect; there can be any number of base pair mismatches. In some embodiments, when two sequences are referred to as “substantially complementary” herein, it is meant that the sequences are sufficiently complementary to each other to hybridize under the selected reaction conditions. Accordingly, substantially complementary sequences can refer to sequences with base-pair complementarity of at least 100, 99, 98, 97, 96, 95, 94, 93, 92, 91, 90, 85, 80, 75, 70, 65, 60 percent or more, or any number in between, in a double-stranded region.

The terms “conjoint therapy” and “combination therapy,” as used herein, refer to the administration of two or more therapeutic agents, e.g., combination of modulators of more than one target listed in Table 1, combination of modulators of more than one target listed in Table 2, combination of at least one modulator of at least one target listed in Table 1 and at least one modulator of at least one target listed in Table 2, combination of at least one modulator of at least one target listed in Table 1 and/or Table 2 and an additional therapeutic agent, such as an immune checkpoint therapy, and the like), and combinations thereof. The different agents comprising the combination therapy can be administered concomitant with, prior to, or following, the administration of the other or others. The combination therapy is intended to provide a beneficial (additive or synergistic) effect from the co-action of these therapeutic agents. Administration of these therapeutic agents in combination can be carried out over a defined time period (usually minutes, hours, days, or weeks depending upon the combination selected). In combination therapy, combined therapeutic agent can be applied in a sequential manner, or by substantially simultaneous application.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample can comprise any suitable sample, including but not limited to a sample from subject, such as a subject having monocytes and/or macrophages and/or a control cancer patient (can be a stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control can comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods encompassed by the present invention. In one embodiment, the control can comprise normal or non-cancerous cell/tissue sample. In another preferred embodiment, the control can comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control can comprise normal cells, cells from patients treated with combination chemotherapy, and cells from patients having benign cancer. In another embodiment, the control can also comprise a measured value for example, average level of expression of a particular gene in a population compared to the level of expression of a housekeeping gene in the same population. Such a population can comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control can comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. The methods encompassed by the present invention are not limited to use of a specific cut-off point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The term “cytokine” refers to a substance secreted by certain cells of the immune system and has a biological effect on other cells. Cytokines can be a number of different substances such as interferons, interleukins and growth factors.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of a biomarker-mediated analysis encompassed by the present invention. One example is determining whether to provide targeted therapy against a cancer to provide therapy using an agent encompassed by the present invention that modulates one or more biomarkers. Another example is starting an adjuvant therapy after surgery whose purpose is to decrease the risk of recurrence. Still another example is to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “endotoxin-free” or “substantially endotoxin-free” refers to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art. Endotoxins may be detected using routine techniques known in the art. For example, the limulus amoebocyte lysate assay, which utilizes blood from the horseshoe crab, is a very sensitive assay for detecting presence of endotoxin. In this test, very low levels of LPS may cause detectable coagulation of the limulus lysate due a powerful enzymatic cascade that amplifies this reaction. Endotoxins may also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/ml, or any range in between, inclusive, such as 0.05 to 10 EU/ml. Typically, 1 ng lipopolysaccharide (LPS) corresponds to about 1-10 EU.

The term “expression signature” or “signature” refers to a group of one or more expressed biomarkers indicative of a state of interest. For example, the genes, proteins, and the like making up this signature can be expressed in a specific cell lineage, stage of differentiation, or during a particular biological response. The biomarkers can reflect biological aspects of the tumors in which they are expressed, such as the inflammatory state of a cell, the cell of origin of a cancer, the nature of a non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

The term “gene” encompasses a nucleotide (e.g., DNA) sequence that encodes a molecule (e.g., RNA, protein, etc.) that has a function. A gene generally comprises two complementary nucleotide strands (i.e., dsDNA), a coding strand and a non-coding strand. When referring to DNA transcription, the coding strand is the DNA strand whose base sequence corresponds to the base sequence of the RNA transcript produced (although with thymine replaced by uracil). The coding strand contains codons, while the non-coding strand contains anticodons. During transcription, RNA Pol II binds the non-coding strand, reads the anti-codons, and transcribes their sequence to synthesize an RNA transcript with complementary bases. In some embodiments, the gene sequence (i.e., DNA sequence) listed is the sequence of the coding strand.

The term “gene product” (also referred to herein as “gene expression product” or “expression product”) encompasses products resulting from expression of a gene, such as nucleic acids (e.g., mRNA) transcribed from the gene, and polypeptides or proteins arising from translation of such mRNA. It will be appreciated that certain gene products can undergo processing or modification, e.g, in a cell. For example, mRNA transcripts can be spliced, polyadenylated, etc., prior to translation, and/or polypeptides can undergo co-translational or post-translational processing, such as removal of secretion signal sequences, removal of organelle targeting sequences, or modifications such as phosphorylation, glycosylation, methylation, fatty acylation, etc. The term “gene product” encompasses such processed or modified forms. Genomic mRNA and polypeptide sequences from a variety of species, including human, are known in the art and are available in publicly accessible databases such as those available at the National Center for Biotechnology Information (ncbi.nih.gov) or Universal Protein Resource (uniprot.org). Other databases include, e.g., GenBank, RefSeq, Gene, UniProtKB/SwissProt, UniProtKB/Trembl, and the like. In general, sequences in the NCBI Reference Sequence database can be used as gene product sequences for a gene of interest. It will be appreciated that multiple alleles of a gene can exist among individuals of the same species. Multiple isoforms of certain proteins can exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments can be directed to particular sequence(s), e.g., particular allele(s) or isoform(s).

The term “generating” encompasses any manner in which a desired result is achieved, such as by direct or indirect action. For example, cells having modulated phenotypes described herein can be generated by direct action, such as by contact with at least one agent that modulates one or more biomarkers described herein, and/or by indirect action, such as by propagating cells having a desired physical, genetic, and/or phenotypic attributes.

The terms “high,” “low,” “intermediate,” and “negative” in connection with cellular biomarker expression refers to the amount of the biomarker expressed relative to the cellular expression of the biomarker by one or more reference cells. Biomarker expression can be determined according to any method described herein including, without limitation, an analysis of the cellular level, activity, structure, and the like, of one or more biomarker genomic nucleic acids, ribonucleic acids, and/or polypeptides. In one embodiment, the terms refer to a defined percentage of a population of cells expressing the biomarker at the highest, intermediate, or lowest levels, respectively. Such percentages can be defined as the top 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 11%, 12%, 13%, 14%, 15% or more, or any range in between, inclusive, of a population of cells that either highly express or weakly express the biomarker. The term “low” excludes cells that do not detectably express the biomarker, since such cells are “negative” for biomarker expression. The term “intermediate” includes cells that express the biomarker, but at levels lower than the population expressing it at the “high” level. In another embodiment, the terms can also refer to, or in the alternative refer to, cell populations of biomarker expression identified by qualitative or statistical plot regions. For example, cell populations sorted using flow cytometry can be discriminated on the basis of biomarker expression level by identifying distinct plots based on detectable moiety analysis, such as based on mean fluorescence intensities and the like, according to well-known methods in the art. Such plot regions can be refined according to number, shape, overlap, and the like based on well-known methods in the art for the biomarker of interest. In still another embodiment, the terms can also be determined according to the presence or absence of expression for additional biomarkers.

The term “substantially identical” refers to a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence. “Substantial identity” can be used to refer to various types and lengths of sequence, such as full-length sequence, functional domains, coding and/or regulatory sequences, exons, introns, promoters, and genomic sequences. Percent sequence identity between two polypeptides or nucleic acid sequences is determined in various ways that are within the skill in the art, for instance, using publicly available computer software such as BLAST program (Basic Local Alignment Search Tool; (Altschul et al. (1995) J. Mol. Biol. 215:403-410), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

The term “immune cell” refers to a cell that is capable of participating, directly or indirectly, in an immune response. Immune cells include, but are not limited to T cells, B cells, antigen presenting cells, dendritic cells, natural killer (NK) cells, natural killer T (NK) cells, lymphokine-activated killer (LAK) cells, monocytes, macrophages, eosinophils, basophils, neutrophils, granulocytes, mast cells, platelets, Langerhan's cells, stem cells, peripheral blood mononuclear cells, cytotoxic T cells, tumor infiltrating lymphocytes (TIL), and the like. An “antigen presenting cell” (APC) is a cell that are capable of activating T cells, and includes, but is not limited to, monocytes/macrophages, B cells and dendritic cells (DCs). The term “dendritic cell” or “DC” refers to any member of a diverse population of morphologically similar cell types found in lymphoid or non-lymphoid tissues. These cells are characterized by their distinctive morphology and high levels of surface MHC-class II expression. DCs can be isolated from a number of tissue sources. DCs have a high capacity for sensitizing MHC-restricted T cells and are very effective at presenting antigens to T cells in situ. The antigens can be self-antigens that are expressed during T cell development and tolerance, and foreign antigens that are present during normal immune processes. The term “neutrophil” generally refers to a white blood cell that makes up part of the innate immune system. Neutrophils typically have segmented nucleic containing about 2-5 lobes. Neutrophils frequently migrate to the site of an injury within minutes following trauma. Neutrophils function by releasing cytotoxic compounds, including oxidants, proteases, and cytokines, at a site of injury or infection. The term “activated DC” is a DC that has been pulsed with an antigen and capable of activating an immune cell. The term “NK cell” has its general meaning in the art and refers to a natural killer (NK) cell. One skilled in the art can easily identify NK cells by determining for instance the expression of specific phenotypic marker (e.g., CD56) and identify its function based on, for example, the ability to express different kind of cytokines or the ability to induce cytotoxicity. The term “B cell” refers to an immune cell derived from the bone marrow and/or spleen. B cells can develop into plasma cells which produce antibodies. The term “T cell” refers to a thymus-derived immune cell that participates in a variety of cell-mediated immune reactions, including CD8+ T cell and CD4+ T cell. Conventional T cells, also known as Tconv or Teffs, have effector functions (e.g., cytokine secretion, cytotoxic activity, anti-self-recognition, and the like) to increase immune responses by virtue of their expression of one or more T cell receptors. Tconv or Teffs are generally defined as any T cell population that is not a Treg and include, for example, naïve T cells, activated T cells, memory T cells, resting Tconv, or Tconv that have differentiated toward, for example, the Th1 or Th2 lineages. In some embodiments, Teffs are a subset of non-regulatory T cells (Tregs). In some embodiments, Teffs are CD4+ Teffs or CD8+ Teffs, such as CD4+ helper T lymphocytes (e.g., Th0, Th1, Tfh, or Th17) and CD8+ cytotoxic T cells (lymphocytes). As described further herein, cytotoxic T cells are CD8+T lymphocytes. “Naïve Tconv” are CD4⁺ T cells that have differentiated in bone marrow, and successfully underwent a positive and negative processes of central selection in a thymus, but have not yet been activated by exposure to an antigen. Naïve Tconv are commonly characterized by surface expression of L-selectin (CD62L), absence of activation markers such as CD25, CD44 or CD69, and absence of memory markers such as CD45RO. Naïve Tconv are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tconv are not anergic and can proliferate in response to antigen-based T cell receptor activation (Lechler et al. (2001) Philos. Trans. R Soc. Lond. Biol. Sci. 356:625-637). In tumors, exhausted cells can present hallmarks of anergy.

The term “immunoregulator” refers to a substance, an agent, a signaling pathway or a component thereof that regulates an immune response. The terms “regulating,” “modifying,” or “modulating” with respect to an immune response refer to any alteration in a cell of the immune system or in the activity of such cell. Such regulation includes stimulation or suppression of the immune system (or a distinct part thereof), which can be manifested by an increase or decrease in the number of various cell types, an increase or decrease in the activity of these cells, or any other changes which can occur within the immune system. Both inhibitory and stimulatory immunoregulators have been identified, some of which can have enhanced function in the cancer microenvironment.

The term “immune response” means a defensive response a body develops against a “foreigner,” such as bacteria, viruses, and pathogens, as well as against targets that may not necessarily originate outside the body, including, without limitation, a defensive response against substances naturally present in the body (e.g., autoimmunity against self-antigens) or against transformed (e.g., cancer) cells. An immune response in particular is the activation and/or action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies (humoral response), cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues. An anti-cancer immune response refers to an immune surveillance mechanism by which a body recognizes abnormal tumor cells and initiates both the innate and adaptive of the immune system to eliminate dangerous cancer cells.

The innate immune system is a non-specific immune system that comprises the cells (e.g., natural killer cells, mast cells, eosinophils, basophils; and the phagocytic cells including macrophages, neutrophils, and dendritic cells) and mechanisms that defend the host from infection by other organisms. An innate immune response can initiate the productions of cytokines, and active complement cascade and adaptive immune response. The adaptive immune system is specific immune system that is required and involved in highly specialized systemic cell activation and processes, such as antigen presentation by an antigen presenting cell; antigen specific T cell activation and cytotoxic effect.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” or “downregulate” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. Similarly, a biological function, such as the function of a protein, is inhibited if it is decreased as compared to a reference state, such as a control like a wild-type state. Such inhibition or deficiency can be induced, such as by application of an agent at a particular time and/or place, or can be constitutive, such as by a heritable mutation. Such inhibition or deficiency can also be partial or complete (e.g., essentially no measurable activity in comparison to a reference state, such as a control like a wild-type state). Essentially complete inhibition or deficiency is referred to as blocked. The term “promote” or “upregulate” has the opposite meaning.

The term “interaction,” when referring to an interaction between two molecules, refers to the physical contact (e.g., binding) of the molecules with one another. Generally, such an interaction results in an activity (which produces a biological effect) of one or both of said molecules. The activity can be a direct activity of one or both of the molecules, (e.g., signal transduction). Alternatively, one or both molecules in the interaction can be prevented from binding their ligand, and thus be held inactive with respect to ligand binding activity (e.g., binding its ligand and triggering or inhibiting costimulation). To inhibit such an interaction results in the disruption of the activity of one or more molecules involved in the interaction. To enhance such an interaction is to prolong or increase the likelihood of said physical contact, and prolong or increase the likelihood of said activity.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

The term “isotype” refers to the antibody class (e.g., IgM, IgG1, IgG2C, and the like) that is encoded by heavy chain constant region genes.

The term “K_{D}” is intended to refer to the dissociation equilibrium constant of a particular antibody-antigen interaction. The binding affinity of antibodies of the disclosed invention can be measured or determined by standard antibody-antigen assays, for example, competitive assays, saturation assays, or standard immunoassays such as ELISA or RIA.

The term “microenvironment” generally refers to the localized area in a tissue area of interest and can, for example, refer to a “tumor microenvironment.” The term “tumor microenvironment” or “TME” refers to the surrounding microenvironment that constantly interacts with tumor cells which is conducive to allow cross-talk between tumor cells and its environment. The tumor microenvironment can include the cellular environment of the tumor, surrounding blood vessels, immune cells, fibroblasts, bone marrow derived inflammatory cells, lymphocytes, signaling molecules and the extracellular matrix. The tumor environment can include tumor cells or malignant cells that are aided and influenced by the tumor microenvironment to ensure growth and survival. The tumor microenvironment can also include tumor-infiltrating immune cells, such as lymphoid and myeloid cells, which can stimulate or inhibit the antitumor immune response, and stromal cells such as tumor-associated fibroblasts and endothelial cells that contribute to the tumor's structural integrity. Stromal cells can include cells that make up tumor-associated blood vessels, such as endothelial cells and pericytes, which are cells that contribute to structural integrity (fibroblasts), as well as tumor-associated macrophages (TAMs) and infiltrating immune cells, including monocytes, neutrophils (PMN), dendritic cells (DCs), T and B cells, mast cells, and natural killer (NK) cells. The stromal cells make up the bulk of tumor cellularity, while the dominating cell type in solid tumors is the macrophage.

The term “modulating” and its grammatical equivalents refer to either increasing or decreasing (e.g., silencing), in other words, either up-regulating or down-regulating.

The “normal” level of expression of a biomarker is the level of expression of the biomarker in cells of a subject, e.g., a human patient, not afflicted with a cancer.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

Such “significance” levels can also be applied to any other measured parameter described herein, such as for expression, inhibition, cytotoxicity, cell growth, and the like.

The term “peripheral blood cell subtypes” refers to cell types normally found in the peripheral blood including, but is not limited to, eosinophils, neutrophils, T cells, monocytes, macrophages, NK cells, granulocytes, and B cells.

The term “pre-determined” biomarker amount and/or activity measurement(s) can be a biomarker amount and/or activity measurement(s) used to, by way of example only, evaluate a subject that can be selected for a particular treatment, evaluate a response to a treatment such as one or more modulators of one or more biomarkers described herein and/or evaluate the disease state. A pre-determined biomarker amount and/or activity measurement(s) can be determined in populations of patients, such as those with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject can affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., cell ratios or serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of a desired. Such predictive use of the biomarker can be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g., a human, afflicted with cancer; (3) its absolute or relatively modulated presence or absence in clinical subset of patients with cancer (e.g., those responding to a particular modulator of T-cell mediated cytotoxicity alone or in combination with immunotherapy or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic molecules.

The term “ratio” refers to a relationship between two numbers (e.g., scores, summations, and the like). Although, ratios can be expressed in a particular order (e.g., a to b or a:b), one of ordinary skill in the art will recognize that the underlying relationship between the numbers can be expressed in any order without losing the significance of the underlying relationship, although observation and correlation of trends based on the ratio can be reversed.

The term “receptor” refers to a naturally occurring molecule or complex of molecules that is generally present on the surface of cells of a target organ, tissue or cell type.

The term “cancer response,” “response to immunotherapy,” or “response to modulators of T-cell mediated cytotoxicity/immunotherapy combination therapy” relates to any response of the hyperproliferative disorder (e.g., cancer) to an cancer agent, such as a modulator of T-cell mediated cytotoxicity, and an immunotherapy, preferably to a change in tumor mass and/or volume after initiation of neoadjuvant or adjuvant therapy. Hyperproliferative disorder response can be assessed, for example for efficacy or in a neoadjuvant or adjuvant situation, where the size of a tumor after systemic intervention can be compared to the initial size and dimensions as measured by CT, PET, mammogram, ultrasound or palpation. Responses can also be assessed by caliper measurement or pathological examination of the tumor after biopsy or surgical resection. Response can be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response can be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein can be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality can be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival can be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement can be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects can be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to a cancer response, e.g., in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease can be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest. For example, the CRISPR-Cas system can be used for precise editing of genomic nucleic acids (e.g., for creating non-functional or null mutations). In such embodiments, the CRISPR guide RNA and/or the Cas enzyme can be expressed. For example, a vector containing only the guide RNA can be administered to an animal or cells transgenic for the Cas9 enzyme. Similar strategies can be used (e.g., zinc finger nucleases (ZFNs), transcription activator-like effector nucleases (TALENs), or homing meganucleases (HEs), such as MegaTAL, MegaTev, Tev-mTALEN, CPF1, and the like). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. Numbers 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene encompassed by the present invention, or a fragment thereof, short interfering RNA (siRNA), and small molecules which interfere with or inhibit expression of a target biomarker nucleic acid by RNA interference (RNAi).

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa et al. (1982) Cancer Res. 42:2159-2164) and cell death assays (Weisenthal et al. (1984) Cancer Res. 94:161-173; Weisenthal et al. (1985) Cancer Treat Rep. 69:615-632; Weisenthal et al., In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432: Weisenthal (1994) Contrib. Gynecol. Obstet. 19:82-90). The sensitivity or resistance can also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

“Short interfering RNA” (siRNA), also referred to herein as “small interfering RNA” is defined as an agent which functions to inhibit expression of a target biomarker nucleic acid, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and can contain a 3′ and/or 5′ overhang on each strand having a length of about 0, 1, 2, 3, 4, or nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 17-29 nucleotide, 19-25 nucleotide, etc. region) antisense strand, followed by a 4-10 nucleotide loop (e.g., a 4, 5, 6, 7, 8, 9, or 10 base linker region), and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, can be administered to a patient having or at risk for having cancer, to inhibit expression of a biomarker gene which is overexpressed in cancer and thereby treat, prevent, or inhibit cancer in the subject.

The term “selective modulator” or “selectively modulate” as applied to a biologically active agent refers to the agent's ability to modulate the target, such as a cell population, signaling activity, etc. as compared to off-target cell population, signaling activity, etc. via direct or interact interaction with the target. For example, an agent that selectively inhibits the interaction between a protein and one natural binding partner over another interaction between the protein and another binding partner, and/or such interaction(s) on a cell population of interest, inhibits the interaction at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 2× (times), 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 105×, 110×, 120×, 125×, 150×, 200×, 250×, 300×, 350×, 400×, 450×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, 10000×, or greater, or any range in between, inclusive, against at least one other binding partner. Such metrics are typically expressed in terms of relative amounts of agent required to reduce the interaction/activity by half. Such metrics apply to any other selectivity arrangement, such as binding of a nucleic acid molecule to one or more target sequences.

More generally, the term “selective” refers to a preferential action or function. The term “selective” can be quantified in terms of the preferential effect in a particular target of interest relative to other targets. For example, a measured variable (e.g., modulation of biomarker expression in desired cells versus other cells, the enrichment and/or deletion of desired cells versus other cells, etc.) can be 10%, 20%, 25%, 30%, 35%, 40%, 45%, 50%, 55%, 60%, 65%, 70%, 75%, 80%, 85%, 90%, 95%, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 70-fold, 80-fold, 90-fold, 100-fold, or greater or any range in between inclusive (e.g., 50% to 16-fold), different in a target of interest versus unintended or undesired targets. The same fold analysis can be used to confirm the magnitude of an effect in a given tissue, cell population, measured variable, and/or measured effect, and the like, such as cell ratios, hyperproliferative cell growth rate or volume, cell proliferation rate, etc. cell numbers, and the like.

By contrast, the term “specific” refers to an exclusionary action or function. For example, specific modulation of an interaction between a protein and one binding partner refers to the exclusive modulation of that interaction and not to any significant modulation of the interaction between the protein and another binding partner. In another example, specific binding of an antibody to a predetermined antigen refers to the ability of the antibody to bind to the antigen of interest without binding to other antigens. Typically, the antibody binds with an affinity (K_{D}) of approximately less than 1×10⁻⁷ M, such as approximately less than 10⁻⁸ M, 10⁻⁹ M, 10⁻¹⁰ M, 10⁻¹¹ M, or even lower when determined by surface plasmon resonance (SPR) technology in a BIACORE® assay instrument using an antigen of interest as the analyte and the antibody as the ligand, and binds to the predetermined antigen with an affinity that is at least 1.1, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. In addition, K_{D} is the inverse of KA. The phrases “an antibody recognizing an antigen” and “an antibody specific for an antigen” are used interchangeably herein with the term “an antibody which binds specifically to an antigen.”

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. The term is intended to encompass all stereoisomers, geometric isomers, tautomers, and isotopes of a chemical structure of interest, unless otherwise indicated.

The term “subject” refers to an animal, vertebrate, mammal, or human, especially one to whom an agent is administered, e.g., for experimental, diagnostic, and/or therapeutic purposes, or from whom a sample is obtained or on whom a procedure is performed. In some embodiments, a subject is a mammal, e.g., a human, non-human primate, rodent (e.g., mouse or rat), domesticated animals (e.g., cows, sheep, cats, dogs, and horses), or other animals, such as llamas and camels. In some embodiments, the subject is human. In some embodiments, the subject is a human subject with a cancer. The term “subject” is interchangeable with “patient.”

The term “survival” includes all of the following: survival until mortality, also known as overall survival (wherein said mortality can be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival can be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more agents (e.g., a modulator of biomarkers listed in Table 1 and/or Table 2 and immunotherapy combination therapy) that is greater than the sum of the separate effects of the cancer agents/therapies alone.

The term “target” refers to a gene or gene product that is modulated, inhibited, or silenced by an agent, composition, and/or formulation described herein. A target gene or gene product includes wild-type and mutant forms. Non-limiting, representative lists of targets encompassed by the present invention are provided in Table 1 and Table 2. Similarly, the term “target”, “targets”, or “targeting” used as a verb refers to modulating the activity of a target gene or gene product. Targeting can refer to upregulating or downregulating the activity of a target gene or gene product.

The term “therapeutic effect” encompasses a local or systemic effect in animals, particularly mammals, and more particularly humans, caused by a pharmacologically active substance. The term thus means any substance intended for use in the diagnosis, cure, mitigation, treatment, or prevention of disease or in the enhancement of desirable physical or mental development and conditions in an animal or human. A prophylactic effect encompassed by the term encompasses delaying or eliminating the appearance of a disease or condition, delaying or eliminating the onset of symptoms of a disease or condition, slowing, halting, or reversing the progression of a disease or condition, or any combination thereof.

The term “effective amount” or “effective dose” of an agent (including a composition and/or formulation comprising such an agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose, or through use of multiple doses, in various embodiments. The term “effective amount” can be a “therapeutically effective amount.”

The terms “therapeutically effective amount” refers to that amount of an agent that is effective for producing some desired therapeutic effect in at least a sub-population of cells in an animal at a reasonable benefit/risk ratio applicable to any medical treatment. Toxicity and therapeutic efficacy of subject compounds can be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the ED₅₀ (i.e., the concentration which achieves half-maximal cytotoxic or cytostatic effect on cancer cells) can be measured and can be, for example, at least 10%, 20%, 30%, 400%, 50%, 60%, 70%, 80%, 90%, 100%, 200N, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

The term “tolerance” or “unresponsiveness” includes refractivity of cells, such as immune cells, to stimulation, e.g., stimulation via an activating receptor or a cytokine. Unresponsiveness can occur, e.g., because of exposure to immunosuppressants or exposure to high doses of antigen. Several independent methods can induce tolerance. One mechanism is referred to as “anergy,” which is defined as a state where cells persist in vivo as unresponsive cells rather than differentiating into cells having effector functions. Such refractivity is generally antigen-specific and persists after exposure to the tolerizing antigen has ceased. For example, anergy in T cells is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134). Another mechanism is referred to as “exhaustion.” T cell exhaustion is a state of T cell dysfunction that arises during many chronic infections and cancer. It is defined by poor effector function, sustained expression of inhibitory receptors and a transcriptional state distinct from that of functional effector or memory T cells.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

The term “treat” refers to the therapeutic management or improvement of a condition (e.g, a disease or disorder) of interest. Treatment can include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment can include reversing, alleviating, reducing severity of, delaying the onset of, curing, inhibiting the progression of, and/or reducing the likelihood of occurrence or recurrence of the disease or one or more symptoms or manifestations of the disease. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. A therapeutic agent can be administered to a subject who has a disease or is at increased risk of developing a disease relative to a member of the general population. In some embodiments, a therapeutic agent can be administered to a subject who has had a disease but no longer shows evidence of the disease. The agent can be administered e.g., to reduce the likelihood of recurrence of evident disease. A therapeutic agent can be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject can have been identified as being at risk of developing the disease (e.g., at increased risk relative to the general population or as having a risk factor that increases the likelihood of developing the disease.

The term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g, IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the API sequence that can be found within the enhancer (Kang et al. (1992) Science 257:1134).

The term “vaccine” refers to a composition for generating immunity for the prophylaxis and/or treatment of diseases.

In addition, there is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, |
| | CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, |
| | TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, |
| | TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well-known feature of the genetic code is its redundancy, whereby, for most of the amino acids used to make proteins, more than one coding nucleotide triplet can be employed (illustrated above). Therefore, a number of different nucleotide sequences can code for a given amino acid sequence. Such nucleotide sequences are considered functionally equivalent since they result in the production of the same amino acid sequence in all organisms (although certain organisms can translate some sequences more efficiently than they do others). Moreover, occasionally, a methylated variant of a purine or pyrimidine can be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Monocytes and Macrophages

Monocytes are myeloid-derived immune effector cells that circulate in the blood, bone marrow, and spleen and have limited proliferation in a steady state condition. The term “myeloid cells” can refer to a granulocyte or monocyte precursor cell in bone marrow or spinal cord, or a resemblance to those found in the bone marrow or spinal cord. The myeloid cell lineage includes circulating monocytic cells in the peripheral blood and the cell populations that they become following maturation, differentiation, and/or activation. These populations include non-terminally differentiated myeloid cells, myeloid derived suppressor cells, and differentiated macrophages. Differentiated macrophages include non-polarized and polarized macrophages, resting and activated macrophages. Without being limiting, the myeloid lineage can also include granulocytic precursors, polymorphonuclear derived suppressor cells, differentiated polymorphonuclear white blood cells, neutrophils, granulocytes, basophils, eosinophils, monocytes, macrophages, microglia, myeloid derived suppressor cells, dendritic cells and erythrocytes. Monocytes are found among peripheral blood mononuclear cells (PBMCs), which also comprise other hematopoietic and immune cells, such as B cells, T cells, NK cells, and the like. Monocytes are produced by the bone marrow from hematopoietic stem cell precursors called monoblasts. Monocytes have two main functions in the immune system: (1) they can exit the bloodstream to replenish resident macrophages and dendritic cells (DCs) under normal states, and (2) they can quickly migrate to sites of infection in the tissues and divide/differentiate into macrophages and inflammatory dendritic cells to elicit an immune response in response to inflammation signals. Monocytes are usually identified in stained smears by their large bilobate nucleus. Monocytes also express chemokine receptors and pathogen recognition receptors that mediate migration from blood to tissues during infection. They produce inflammatory cytokines and phagocytose cells. In some embodiments, monocytes and/or macrophages of interest are identified according to CD11b+ expression and/or CD14+ expression.

As described in detail below, monocytes can differentiate into macrophages. Monocytes can also differentiate into dendritic cells, such as through the action of the cytokines granulocyte macrophage colony-stimulating factor (GM-CSF) and interleukin 4 (IL-4). In general, the term “monocytes” encompasses undifferentiated monocytes, as well as cell types that are differentiated therefrom, including macrophages and dendritic cells. In some embodiments, the term “monocytes” can refer to undifferentiated monocytes.

Macrophages are critical immune effectors and regulators of inflammation and the innate immune response. Macrophages are heterogeneous, tissue-resident, terminally-differentiated, innate myeloid cells, which have remarkable plasticity and can change their physiology in response to local cues from the microenvironment and can assume a spectrum of functional requirements from host defense to tissue homeostasis (Ginhoux et al. (2016) Nat. Immunol. 17:34-40). Macrophages are present in virtually all tissues in the body. They are either tissue resident macrophages, for example Kupffer cells that reside in liver, or derived from circulating monocytic precursors (i.e., monocytes) which mainly originate from bone marrow and spleen reservoirs and migrate into tissue in the steady state or in response to inflammation or other stimulating cues. For example, monocytes can be recruited from the blood to tissue to replenish tissue specific macrophages of the bone, alveoli (lung), central nervous system, connective tissues, gastrointestinal tract, live, spleen and peritoneum.

The term “tissue-resident macrophages” refers to a heterogeneous populations of immune cells that fulfill tissue-specific and/or micro-anatomical niche-specific functions such as tissue immune-surveillance, response to infection and the resolution of inflammation, and dedicated homeostatic functions. Tissue resident macrophages originate in the yolk sac of the embryo and mature in one particular tissue in the developing fetus, where they acquire tissue-specific roles and change their gene expression profile. Local proliferation of tissue resident macrophages, which maintain colony-forming capacity, can directly give rise to populations of mature macrophages in the tissue. Tissue resident macrophages can also be identified and named according to the tissues they occupy. For example, adipose tissue macrophages occupy adipose tissue, Kupffer cells occupy liver tissue, sinus histiocytes occupy lymph nodes, alveolar macrophages (dust cells) occupy pulmonary alveoli, Langerhans cells occupy skin and mucosal tissue, histiocytes leading to giant cells occupy connective tissue, microglia occupy central nervous system (CNS) tissue, Hofbauer cells occupy placental tissue, intraglomerular mesangial cells occupy kidney tissue, osteoclasts occupy bone tissue, epithelioid cells occupy granulomas, red pulp macrophages (sinusoidal lining cells) occupy the red pulp of spleen tissue, peritoneal cavity macrophages occupy peritoneal cavity tissue, lysomac cells occupy Peyer's patch tissue, and pancreatic macrophages occupy pancreatic tissue.

Macrophages, in addition to host defense against infectious agents and other inflammation reaction, can perform different homeostatic functions, including but not limited to, development, wound healing and tissue repairing, and regulation of immune response. Macrophages, first recognized as phagocytosis cells in the body which defend infections through phagocytosis, are essential components of innate immunity. In response to pathogens and other inflammation stimuli, activated macrophages can engulf infected bacteria and other microbes; stimulate inflammation and release a cocktail of pro-inflammatory molecules to these intracellular microorganisms. After engulfing the pathogens, macrophages present pathogenic antigens to T cells to further activate adaptive immune response for defense. Exemplary pro-inflammatory molecules include cytokines IL-1β, IL-6 and TNF-α, chemokine MCP-1, CXC-5 and CXC-6, and CD40L.

In addition to their contribution to host defense against infections, macrophages play vital homeostatic roles, independent of their involvement in immune responses. Macrophages are prodigious phagocytic cells that clear erythrocytes and the released substances such as iron and hemoglobin can be recycled for the host to reuse. This clearance process is a vital metabolic contribution without which the host would not survive.

Macrophages are also involved in the removal of cellular debris that is generated during tissue remodeling, and rapidly and efficiently clear cells that have undergone apoptosis. Macrophages are believed to be involved in steady-state tissue homeostasis via the clearance of apoptotic cells. These homeostatic clearance processes are generally mediated by surface receptors on macrophages including scavenger receptors, phosphatidyl serine receptors, the thrombospondin receptor, integrins and complement receptors. These receptors that mediate phagocytosis either fail to transduce signals that induce cytokine-gene transcription or actively produce inhibitory signals and/or cytokines. The homeostatic function of macrophages is independent of other immune cells.

Macrophages can also clear cellular debris/necrotic cells that results from trauma or other damages to cells. Macrophages detect the endogenous danger signals that are present in the debris of necrotic cells through toll-like receptors (TLRs), intracellular pattern-recognition receptors and the interleukin-1 receptor (IL-IR), most of which signal through the adaptor molecule myeloid differentiation primary-response gene 88 (MyD88). The clearance of cellular debris can markedly alter the physiology of macrophages. Macrophages that clear necrosis can undergo dramatic changes in their physiology, including alterations in the expression of surface proteins and the production of cytokines and pro-inflammatory mediators. The alterations in macrophage surface-protein expression in response to these stimuli could potentially be used to identify biochemical markers that are unique to these altered cells.

Macrophages have important functions in maintaining homeostasis in many tissues such as white adipose tissue, brown adipose tissue, liver and pancreas. Tissue macrophages can quickly respond to changing conditions in a tissue, by releasing cell signaling molecules that trigger a cascade of changes allowing tissue cells to adapt. For instance, macrophages in adipose tissue regulate the production of new fat cells in response to changes in diet (e.g., macrophages in white adipose tissue) or exposure to cold temperatures (e.g., macrophages in brown adipose tissue). Macrophages in the liver, known as Kupffer cells, regulate the breakdown of glucose and lipids in response to dietary changes. Macrophages in pancreas can regulate insulin production in response to high fat diet.

Macrophages can also contribute to wound healing and tissue repair. For example, macrophages, in response to signals derived from injured tissues and cells, can be activated and induce a tissue-repair response to repair damaged tissue (Minutti et al. (2017) Science 356:1076-1080).

During embryonic development, macrophages also play a key role in tissue remodeling and organ development. For example, resident macrophages actively shape the development of blood vessels in neonatal mouse hearts (Leid et al. (2016) Circ. Res. 118:1498-1511). Microglia in the brain can produce growth factors that guide neurons and blood vessels in developing brain during embryonic development. Similarly, CD95L, a macrophage-produced protein, binds to CD95 receptors on the surface of neurons and developing blood vessels in the brains of mouse embryos and increases neuron and blood vessel development (Chen et al. (2017) Cell Rep. 19:1378-1393). Without the ligand, neurons branch less frequently, and the resulting adult brain exhibits less electrical activity Monocyte-derived cells known as osteoclasts are involved in bone development, and mice that lack these cells develop dense, hardened bones—a rare condition known as osteopetrosis. Macrophages also orchestrate development of the mammary gland and assist in retinal development in the early postnatal period (Wynn et al. (2013) Nature 496:445-455).

As described above, macrophages regulate immune systems. In addition to the presentation of antigens to T cells, macrophages can provide immunosuppressive/inhibitory signals to immune cells in some conditions. For example, in the testis, macrophages help create a protective environment for sperm from being attacked by the immune system. Tissue resident macrophages in the testis produce immunosuppressant molecules that prevent immune cell reaction against sperm (Mossadegh-Keller et al. (2017) J. Ep. Med. 214:10.1084/jem.20170829).

The plasticity of macrophages in response to different environment signals and in agreement with their functional requirements has resulted in a spectrum of macrophage activation states, including two extremes of the continuum, namely “classically activated” M1 and “alternatively activated” M2 macrophages.

The term “activation” refers to the state of a monocyte and/or macrophage that has been sufficiently stimulated to induce detectable cellular proliferation and/or has been stimulated to exert its effector function, such as induced cytokine expression and secretion, phagocytosis, cell signaling, antigen processing and presentation, target cell killing, and pro-inflammatory function.

The term “M1 macrophages” or “classically activated macrophages” refers to macrophages having a pro-inflammatory phenotype. The term “macrophage activation” (also referred to as “classical activation”) was introduced by Mackaness in the 1960s in an infection context to describe the antigen-dependent, but non-specific enhanced, microbicidal activity of macrophages toward BCG (bacillus Calmette-Guerin) and Listeria upon secondary exposure to the pathogens (Mackaness (1962). Exp. Med. 116:381-406). The enhancement was later linked with Thl responses and IFN-γ production by antigen-activated immune cells (Nathan et al. (1983) J. Exp. Med. 158:670-689) and extended to cytotoxic and antitumoral properties (Pace et al. (1983) Proc. Nat. Acad. Sci. U.S.A. 80:3782-3786; Celada et al. (1984) J. Exp. Med 160:55-74). Therefore, any macrophage functionality that enhances inflammation by cytokine secretion, antigen presentation, phagocytosis, cell-cell interactions, migration, etc. is considered pro-inflammatory., vitro and in vivo assays can measure different endpoints: general in vitro measurements include pro-inflammatory cell stimulation as measured by proliferation, migration, pro-inflammatory Th1 cytokine/chemokine secretion and/or migration, while general in vivo measurements further include analyzing pathogen fighting, tissue injury immediate responders, other cell activators, migration inducers, etc. For both in vitro and in vivo, pro-inflammatory antigen presentation can be assessed. Bacterial moieties, such as lipopolysaccharide (LPS), certain Toll-like receptor (TLR) agonists, the Th1 cytokine interferon-gamma (IFNγ) (e.g., IFNγ produced by NK cells in response to stress and infections, and T helper cells with sustained production) and TNF polarize macrophages along the M1 pathway. Activated M1 macrophages phagocytose and destroy microbes, eliminate damaged cells (e.g., tumor cells and apoptotic cells), present antigen to T cells for increasing adaptive immune responses, and produce high levels of pro-inflammatory cytokines (e.g., IL-1, IL-6, and IL-23), reactive oxygen species (ROS), and nitric oxide (NO), as well as activate other immune and non-immune cells. Characterized by their expression of inducible nitric oxide synthase (iNOS), reactive oxygen species (ROS), and production of the Th1-associated cytokine, IL-12, M1 macrophages are well-adapted to promote a strong immune response. The metabolism of M1 macrophages is characterized by enhanced aerobic glycolysis, converting glucose into lactate, increased flux through the pentose phosphate pathway (PPP), fatty acid synthesis, and a truncated tricarboxylic acid (TCA) cycle, leading to accumulation of succinate and citrate.

A “Type 1” or “M1-like” monocyte and/or macrophage is a monocyte and/or macrophage capable of contributing to a pro-inflammatory response that is characterized by at least one of the following: producing inflammatory stimuli by secreting at least one pro-inflammatory cytokine, expressing at least one cell surface activating molecule/a ligand for an activating molecule on its surface, recruiting/instructing/interacting with at least one other cell (including other macrophages and/or T cells) to stimulate pro-inflammatory responses, presenting antigen in a pro-inflammatory context, migrating to the site allowing for pro-inflammatory response initiation or starting to express at least one gene that is expected to lead to pro-inflammatory functionality. In some embodiments, the term includes activating cytotoxic CD8+ T cells, mediating increased sensitivity of cancer cells to immunotherapy, such as immune checkpoint therapy, and/or mediating reversal of cancer cells to resistance. In certain embodiments, such modulation toward a pro-inflammatory state can be measured in a number of well-known manners, including, without limitation, one or more of a) increased cluster of differentiation 80 (CD80), CD86, MHCII, MHCI, interleukin 1-beta (IL-1β, IL-6, CCL3, CCL4, CXCL10, CXCL9, GM-CSF and/or tumor necrosis factor alpha (TNF-α); b) decreased expression and/or secretion of CD206, CD163, CD16, CD53, VSIG4, PSGL-1, TGFb and/or IL-10; c) increased secretion of at least one cytokine or chemokine selected from the group consisting of IL-1β, TNF-α, IL-12, IL-18, GM-CSF, CCL3, CCL4, and IL-23; d) increased ratio of expression of IL-1β, IL-6, and/or TNF-α to expression of IL-10; e) increased CD8+ cytotoxic T cell activation; f) increased recruitment of CD8+ cytotoxic T cell activation; g) increased CD4+ helper T cell activity; h) increased recruitment of CD4+ helper T cell activity; i) increased NK cell activity; j) increased recruitment of NK cell; k) increased neutrophil activity; 1) increased macrophage activity; and/or m) increased spindle-shaped morphology, flatness of appearance, and/or number of dendrites, as assessed by microscopy.

In cells that are already pro-inflammatory, an increased inflammatory phenotype refers to an even more pro-inflammatory state.

By contrast, the term “M2 macrophages” refers to macrophages having an anti-inflammatory phenotype. Th2- and tumor-derived cytokines, such as IL-4, IL-10, IL-13, transforming growth factor beta (TGF-β), or prostaglandin E2 (PGE2) can promulgate M2 polarization. The metabolic profile of M2 macrophages is defined by OXPHOS, FAO, a decreased glycolysis, and PPP. The discovery that the mannose receptor was selectively enhanced by the Th2 IL-4 and IL-13 in murine macrophages, and induced high endocytic clearance of mannosylated ligands, increased major histocompatibility complex (MHC) class II antigen expression, and reduced pro-inflammatory cytokine secretion, led Stein, Doyle, and colleagues to propose that IL-4 and IL-13 induced an alternative activation phenotype, a state altogether different from IFN-γ activation but far from deactivation (Martinez and Gordon (2014) F1000 Prime Reports 6:13). In vitro and in vivo definition/assays can measure different endpoints: general in vitro endpoints include anti-inflammatory cell stimulation measured by proliferation, migration, anti-inflammatory Th2 cytokine/chemokine secretion and/or migration, while general in vivo M2 endpoints further include analyzing pathogen fighting, tissue injury delayed/pro-fibrotic response, other cell Th2 polarization, migration inducers, etc. For both in vitro and in vivo, pro-tolerogenic antigen presentation can be assessed.

A “Type 2” or “M2-like” monocyte and/or macrophage is a monocyte and/or macrophage capable of contributing to an anti-inflammatory response that is characterized by at least one of the following: producing anti-inflammatory stimuli by secreting at least one anti-inflammatory cytokine, expressing at least one cell surface inhibiting molecule/ligand for an inhibitory molecule on its surface, recruiting/instructing/interacting at least one other cell to stimulate anti-inflammatory responses, presenting antigen in a pro-tolerogenic context, migrating to the site allowing for pro-tolerogenic response initiation or starting to express at least one gene that is expected to lead to pro-tolerogenic/anti-inflammatory functionality. In certain embodiments, such modulation toward a pro-inflammatory state can be measured in a number of well-known manners, including, without limitation, the opposite of the Type 1 pro-inflammatory state measurements described above.

A cell that has an “increased inflammatory phenotype” is one that has a more pro-inflammatory response capacity related to a) an increase in one or more of the Type 1 listed-criteria and/or b) a decrease in one or more of the Type 2-listed criteria, after modulation of at least one biomarker (e.g., at least one target listed in Table 1 and/or Table 2) of the present invention, such as contact by an agent that modulates the at least one biomarker (e.g., at least one target listed in Table 1 and/or Table 2) of the present invention.

A cell that has a “decreased inflammatory phenotype” is one that has a more anti-inflammatory response capacity related to a) an decrease in one or more of the Type 1 listed-criteria and/or b) an increase of one or more of the Type 2-listed criteria, after modulation of at least one biomarker (e.g., at least one target listed in Table 1 and/or Table 2) of the present invention, such as contact by an agent that modulates the at least one biomarker (e.g., at least one target listed in Table 1 and/or Table 2) of the present invention.

Thus, macrophages can adopt a continuum of alternatively activated states with intermediate phenotypes between the Type 1 and Type 2 states (see, e.g., Biswas et al. (2010) Nat. Immunol. 11: 889-896; Mosser and Edwards (2008) Nat. Rev. Immunol. 8:958-969; Mantovani et al. (2009) Hum. Immunol. 70:325-330) and such increased or decreased inflammatory phenotypes can be determined as described above.

As used herein, the term “alternatively activated macrophages” or “alternatively activated states” refers to essentially all types of macrophage populations other than the classically activated M1 pro-inflammatory macrophages. Originally, the alternatively activated state was designated only to M2 type anti-inflammatory macrophages. The term has expanded to include all other alternative activation states of macrophages with dramatic difference in their biochemistry, physiology and functionality.

For example, one type of alternatively activated macrophages is those involved in wound healing. In response to innate and adaptive signals released during tissue injury (e.g., surgical wound), such as IL-4 produced by basophils and mast cells, tissue-resident macrophages can be activated to promote wound healing. The wound healing macrophages, instead of producing high levels of pro-inflammatory cytokines, secret large amounts of extracellular matrix components, e.g., chitinase and chitinase-like proteins YM1/CHI3L3, YM2, AMCase and Stabilin, all of which exhibit carbohydrate and matrix-binding activities and involve in tissue repair.

Another example of alternatively activated macrophages involves regulatory macrophages that can be induced by innate and adaptive immune response. Regulatory macrophages can contribute to immuno-regulatory function. For example, macrophages can respond to hormones from the hypothalamic-pituitary-adrenal (HPA) axis (e.g., glucocorticoids) to adopt a state with inhibited host defense and inflammatory function such as inhibition of the transcriptions of pro-inflammatory cytokines. Regulatory macrophages can produce regulatory cytokine TGF-P to dampen immune responses in certain conditions, for instance, at late stage of adaptive immune response. Many regulatory macrophages can express high levels of co-stimulatory molecules (e.g., CD80 and CD86) and therefore enhance antigen presentation to T cells.

Many stimuli/cues can induce polarization of regulatory macrophages. The cues can include, but are not limited to, the combination of TLR agonist and immune complexes, apoptotic cells, IL-10, prostaglandins, GPcR ligands, adenosine, dopamine, histamine, sphingosine1-phosphate, melanocortin, vasoactive intestinal peptides and Siglec-9. Some pathogens, such as parasites, viruses, and bacteria, can specifically induce the differentiation of regulatory macrophages, resulting in defective pathogen killing and enhanced survival and spread of the infected microorganisms.

Regulatory macrophages share some common features. For example, regulatory macrophages need two stimuli to induce their anti-inflammatory activity. Differences among the regulatory macrophage subpopulations that are induced by different cues/stimuli are also observed, reflecting their heterogeneity.

Regulatory macrophages also are a heterogeneous population of macrophages, including a variety of subpopulations found in metabolism, during development, in the maintenance of homeostasis. In one example, a subpopulation of alternatively activated macrophages are immunoregulatory macrophages with unique immunoregulatory properties which can be induced in the presence of M-CSF/GM-CSF, a CD16 ligand (such as an immunoglobulin), and IFN-γ (PCT application publication NO. WO2017/153607).

Macrophages in a tissue can change their activation states in vivo over time. This dynamic reflects constant influx of migrating macrophages to the tissue, dynamic changes of activated macrophages, and macrophages that switch back the rest state. In some conditions, different signals in an environment can induce macrophages to a mix of different activation states. For example, in a condition with chronic wound, macrophages over time, can include pro-inflammatory activation subpopulation, macrophages that are pro-wound healing, and macrophages that exhibit some pro-resolving activities. Under non-pathological conditions, a balanced population of immune-stimulatory and immune-regulatory macrophages exist in the immune system. In some disease conditions, the balance is interrupted and the imbalance causes many clinical conditions.

The apparent plasticity of macrophages also make them vulnerably responsive to environmental cues they receive in a disease condition. Macrophages can be repolarized in response to a variety of disease conditions, demonstrating distinct characteristics. One example is macrophages that are attracted and filtrate into tumor tissues from peripheral blood monocytes, which are often called “tumor associated macrophages” (“TAMs”) or “tumor infiltrating macrophages” (“TIMs”). Tumor-associated macrophages are amongst the most abundant inflammatory cells in tumors and a significant correlation was found between high TAM density and a worse prognosis for most cancers (Zhang et al. (2012) PloS One 7:e50946.10.1371/journal.pone.0050946).

TAMs are a mixed population of both M1-like pro-inflammatory and M2-like anti-inflammatory subpopulations. In the earliest stage of neoplasia, classically activated macrophages that have a pro-inflammatory phenotype are present in the normoxic tumor regions, are believed to contribute to early eradication of transformed tumor cells. However, as a tumor grows and progresses, the majority of TAMs in late stage tumors is M2-like regulatory macrophages that reside in the hypoxic regions of the tumor. This phenotypic change of macrophages is markedly influenced by the tumor microenvironmental stimuli, such as tumor extracellular matrix, anoxic environment and cytokines secreted by tumor cells. The M2-like TAMs demonstrate a hybrid activation state of wound healing macrophages and regulatory macrophages, demonstrating various unique characteristics, including the production of high levels of IL-10 but little or no IL-12, defective TNF production, suppression of antigen presenting cells, and contribution to tumor angiogenesis.

Generally, TAMs are characterized by a M2 phenotype and suppress M1 macrophage-mediated inflammation through IL-10 and IL-1p production. Thus, TAMs promote tumor growth and metastasis through activation of wound-healing (i.e., anti-inflammatory) pathways that provide nutrients and growth signals for proliferation and invasion and promote the creation of new blood vessels (i.e., angiogenesis). In addition, TAMs contribute to the immune-suppressive tumor microenvironment by secreting anti-inflammatory signals that prevent other components of the immune system from recognizing and attacking the tumor. It has been reported that TAMs are key players in promoting cancer growth, proliferation, and metastasis in many types of cancers (e.g., breast cancer, astrocytoma, head and neck squamous cell cancer, papillary renal cell carcinoma Type II, lung cancer, pancreatic cancer, gall bladder cancer, rectal cancer, glioma, classical Hodgkin's lymphoma, ovarian cancer, and colorectal cancer). In general, a cancer characterized by a large population of TAMs is associated with poor disease prognosis.

The diversified functions and activation states can have dangerous consequences if not appropriately regulated. For example, classically activated macrophages can cause damage to host tissue, predispose surrounding tissue and influence glucose metabolism if over activated.

In many disease conditions, the balanced dynamics of macrophage activation states is interrupted and the imbalance causes diseases. For example, tumors are abundantly populated with macrophages. Macrophages can be found in 75 percent of cancers. The aggressive types of cancer are often associated with higher infiltration of macrophages and other immune cells. In most malignant tumors, TAM exert several tumor-promoting functions, including promotion of cancer cell survival, proliferation, invasion, extravasation and metastasis, stimulation of angiogenesis, remodeling of the extracellular matrix, and suppression of antitumor immunity (Qian and Pollard, 2010, Cell, 141(1): 39-51). They also could produce growth-promoting molecules such as ornithine, VEGF, EGF and TGF-β.

TAMs stimulate tumor growth and survival in response to CSF1 and IL4/IL13 encountered in the tumor microenvironment. TAMs also can remodel the tumor microenvironment through the expression of proteases, such as MMPs, cathepsins and uPA and matrix remodeling enzymes (e.g., lysyl oxidase and SPARC).

TAMs play an important role in tumor angiogenesis regulating the dramatic increase of blood vessel in tumor tissues which is required for the transition of the malignant state of tumor. These angiogenic TAMs express angiopoietin receptor, TIE2 and secrete many angiogenic molecules including VEGF family members, TNFα, IL1β, IL8, PDGF and FGF.

A diversity of subpopulations of macrophages perform these individual pro-tumoral functions. These TAMs are different in the extent of macrophage infiltrate as well as phenotype in different tumor types. For example, detailed profiling in human hepatocellular carcinoma shows various macrophage sub-types defined in terms of their anatomic location, and pro-tumoral and anti-tumoral properties. It has been shown that M2-like macrophages are a major resource of pro-tumoral functions of TAMs. M2-like TAMs have been shown to affect the efficacy of anti-cancer treatments, contribute to therapy resistance, and mediate tumor relapse following conventional cancer therapy.

### III. Targets and Biomarkers Useful for Modulating Monocyte and/or Macrophage Inflammatory Phenotype

The present invention encompasses biomarkers (e.g., targets listed in Table 1 and Table 2) useful for modulating the inflammatory phenotype of monocytes and/or macrophages, as well as corresponding immune responses (e.g., to increase anti-cancer macrophage immunotherapy).

Table 1 provides gene information for targets, wherein their downregulation, such as by agents that downregulate the targets like antibodies, siRNAs, and the like described herein, is associated with and results in an increased inflammatory phenotype (e.g., a Type 1 phenotype).

Table 2 provides gene information for targets, wherein their downregulation, such as by agents that downregulate the targets like antibodies, siRNAs, and the like described herein, is associated with and results in a decreased inflammatory phenotype (e.g., a Type 2 phenotype).

Nucleic acid and amino acid sequence information for the loci and biomarkers encompassed by the present invention (e.g., biomarkers listed in Tables 1 and 2) are well-known in the art and readily available on publicly available databases, such as the National Center for Biotechnology Information (NCBI). For example, exemplary nucleic acid and amino acid sequences derived from publicly available sequence databases are provided below.

As discussed further below, agents that modulate the expression, translation, degradation, amount, subcellular localization, and other activities of biomarkers encompassed by the present invention in monocytes and/or macrophages are useful in modulating the inflammatory phenotype of these cells, as well as modulating immune responses mediated by these cells.

Although numerous representative orthologs to human sequences are provided below, in some embodiments, human biomarkers (including modulation and modulatory agents thereof) are preferred. For some biomarkers, it is believed that immune responses mediated by such biomarkers in humans is particularly useful in view of differences between the human immune system and the immune system of other vertebrates.

The term “SIGLEC9” refers to Sialic Acid Binding Ig Like Lectin 9, a putative adhesion molecule that mediates sialic-acid dependent binding to cells. SIGLEC9 preferentially binds to alpha-2,3- or alpha-2,6-linked sialic acid. The sialic acid recognition site may be masked by cis interactions with sialic acids on the same cell surface. Among its related pathways are innate immune system and class I MHC mediated antigen processing and presentation. In some embodiments, the SIGLEC9 gene, located on chromosome 19q in humans, consists of 12 exons. Orthologs are known from chimpanzee, rhesus monkey, and mouse. A knockout mouse line, called Siglec^{tmc1Croc} was generated (McMillan et al. (2013) Blood 121(11):2084-2094). In some embodiments, human SIGLEC9 protein has 463 amino acids and/or has a molecular mass of 50082 Da. In some embodiments, the SIGLEC9 protein contains one copy of a cytoplasmic motif that is referred to as the immunoreceptor tyrosine-based inhibitor motif (ITIM). This motif is involved in modulation of cellular responses. The phosphorylated ITIM motif can bind the SH2 domain of several SH2-containing phosphatases.

The term “SIGLEC9” is intended to include fragments, variants (e.g., allelic variants), and derivatives thereof. Representative human SIGLEC9 cDNA and human SIGLEC9 protein sequences are well-known in the art and are publicly available from the National Center for Biotechnology Information (NCBI) (see, for example, ncbi.nlm.nih.gov/gene/27180). For example, at least two different human SIGLEC9 isoforms are known. Human SIGLEC9 isoform 1 (NP_001185487.1) is encodable by the transcript variant 1 (NM_001198558.1), which is the longer transcript. Human SIGLEC9 isoform 2 (NP_055256.1) is encodable by the transcript variant 2 (NM_014441.2), which differs in the 3 UTR and 3′ coding region compared to isoform 1. The encoded isoform 2 is shorter and has a distinct C-terminus compared to isoform 1. Nucleic acid and polypeptide sequences of SIGLEC9 orthologs in organisms other than humans are well-known and include, for example, chimpanzee SIGLEC9 (XM_024351618.1 and XP_024207386.1, and XM_003316566.5 and XP_003316614.2), rhesus monkey SIGLEC9 (XM_015124691.1 and XP_014980177.1, XM_001114560.3 and XP_001114560.2, XM_015124692.1 and XP_014980178.1), and mouse SIGLEC9 (NM_031181.2 and NP_112458.2). Representative sequences of SIGLEC9 orthologs are presented below in Table 1.

Anti-SIGLEC9 antibodies suitable for detecting SIGLEC9 protein are well-known in the art and include, for example, antibodies MAB1139 and AF1139 (R&D systems, Minneapolis, Minn.), antibodies MAB1139, NBP1-47969, AF1139, NBP2-27070 and NBP1-85755 (Novus Biologicals, Littleton, Colo.), antibodies ab89484, ab96545, and ab197981 (AbCam, Cambridge, Mass.), antibodies Cat #: CF500382 and TA500382 (Origene, Rockville, Md.), etc. Other anti-SIGLEC9 antibodies are also known and include, for example, those described in U.S. Pat. Pubs. US20170306014, US20190085077, US20190023786, and US20180244770. In addition, reagents are well-known for detecting SIGLEC9 expression. Multiple clinical tests of SIGLEC9 are available in NIH Genetic Testing Registry (GTR®) (e.g., GTR Test ID: GTR000547533.2, offered by Fulgent Clinical Diagnostics Lab (Temple City, Calif.)). Moreover, multiple siRNA, shRNA, CRISPR constructs for reducing SIGLEC9 expression can be found in the commercial product lists of the above-referenced companies, such as siRNA product #SR309022, shRNA products #TG309443, TL309443, and CRISPR products #KN206674 from Origene Technologies (Rockville, Md.), CRISPR gRNA products from Santa Cruz (sc-406675 and sc-406675-KO-2), and RNAi products from Santa Cruz (Cat #sc-106550 and sc-153462). It is to be noted that the term can further be used to refer to any combination of features described herein regarding SIGLEC9 molecules. For example, any combination of sequence composition, percentage identify, sequence length, domain structure, functional activity, etc. can be used to describe a SIGLEC9 molecule encompassed by the present invention.

The term “VSIG4” refers to V-Set And Immunoglobulin Domain Containing 4, a v-set and immunoglobulin-domain containing protein that is structurally related to the B7 family of immune regulatory proteins. The VSIG4 protein is a negative regulator of T-cell responses. It is also a receptor for the complement component 3 fragments C3b and iC3b. VSIG4 protein is a phagocytic receptor, and a strong negative regulator of T-cell proliferation and IL2 production. It is also a potent inhibitor of the alternative complement pathway convertases. Diseases associated with VSIG4 include T-Cell/Histiocyte Rich Large B Cell Lymphoma and Langerhans Cell Sarcoma. Among its related pathways are complement and coagulation cascades. In some embodiments, the VSIG4 gene, located on chromosome Xq in humans, consists of 8 exons. Orthologs are known from chimpanzee, rhesus monkey, dog, mouse, and rat. Knockout mouse lines, incluinng Vsig4^{tmlGne}, (Helmy et al. (2006) Cell 124:915-927) and Vsig4^{tmlb(EUCOM)Hmgu} (Skarnes et al. (2011) Nature 474:337-342), exist. In some embodiments, human VSIG4 protein has 399 amino acids and/or a molecular mass of 43987 Da.

The term “VSIG4” is intended to include fragments, variants (e.g., allelic variants), and derivatives thereof. Representative human VSIG4 cDNA and human VSIG4 protein sequences are well-known in the art and are publicly available from the National Center for Biotechnology Information (NCBI) (see, for example, ncbi.nlm.nih.gov/gene/11326). For example, at least five different human VSIG4 isoforms are known. Human VSIG4 isoform 1 (NP_009199.1) is encodable by the transcript variant 1 (NM_007268.2), which is the longest transcript. Human VSIG4 isoform 2 (NP_001093901.1) is encodable by the transcript variant 2 (NM_001100431.1), which lacks an alternate in-frame segment compared to variant 1. Human VSIG4 isoform 3 (NP_001171760.1) is encodable by the transcript variant 3 (NM_001184831.1), which has multiple differences, compared to variant 1. Human VSIG4 isoform 4 (NP_001171759.1) is encodable by the transcript variant 4 (NM_001184830.1), which differs in the 3′ UTR and 3′ coding region, compared to variant 1. Human VSIG4 isoform 5 (NP_001244332.1) is encodable by the transcript variant 5 (NM_001257403.1), which lacks two alternate in-frame exons in the 3′ coding region, compared to variant 1. Nucleic acid and polypeptide sequences of VSIG4 orthologs in organisms other than humans are well-known and include, for example, chimpanzee VSIG4 (NM_001279873.1 and NP_001266802.1), rhesus monkey VSIG4 (XM_015127596.1 and XP_014983082.1, XM_015127593.1 and XP_014983079.1, XM_015127595.1 and XP_014983081.1, XM_001099264.2 and XP_001099264.2, and XM_015127594.1 and XP_014983080.1), dog VSIG4 (XM_005641424.3 and XP_005641481.1; XM_005641423.3 and XP_005641480.1; XM_022416007.1 and XP_022271715.1; XM_005641421.3 and XP_005641478.1; and XM_005641422.3 and XP_005641479.1), mouse VSIG4 (NM_177789.4 and NP_808457.1), and rat VSIG4 (NM_001025004.1 and NP_001020175.1). Representative sequences of VSIG4 orthologs are presented below in Table 1.

Anti-VSIG4 antibodies suitable for detecting VSIG4 protein are well-known in the art and include, for example, antibodies AF4646 and AF4674 (R&D systems, Minneapolis, Minn.), antibodies NBP1-86843, AF4646, AF4674, and NBP1-69631 (Novus Biologicals, Littleton, Colo.), antibodies ab56037, ab197161, and ab138594 (AbCam, Cambridge, Mass.), antibodies Cat #: TA346124 (Origene, Rockville, Md.), antibodies 05 and 202 (Sino Biological, Beijing, China), etc. Other anti-VSIG4 antibodies are also known and include, for example, those described in U.S. Pat. Pubis. US20090162356A1 and US20180371095A1. In addition, reagents are well-known for detecting VSIG4 expression. Multiple clinical tests of VSIG4 are available in NIH Genetic Testing Registry (GTR®) (e.g., GTR Test ID: GTR000544515.2, offered by Fulgent Clinical Diagnostics Lab (Temple City, Calif.)). Moreover, multiple siRNA, shRNA, CRISPR constructs for reducing VSIG4 expression can be found in the commercial product lists of the above-referenced companies, such as siRNA product #SR323415, shRNA products #TG308440, TL308440, TF308440, and CRISPR products #KN203751 from Origene Technologies (Rockville, Md.), CRISPR gRNA products from Applied Biological Materials (K7367508) and from Santa Cruz (sc-404067), and RNAi products from Santa Cruz (Cat #sc-72190 and sc-72196). It is to be noted that the term can further be used to refer to any combination of features described herein regarding VSIG4 molecules. For example, any combination of sequence composition, percentage identify, sequence length, domain structure, functional activity, etc. can be used to describe a VSIG4 molecule encompassed by the present invention.

The term “CD74” refers to CD74. The protein encoded by this gene associates with class II major histocompatibility complex (MHC) and is an important chaperone that regulates antigen presentation for immune response. It also serves as cell surface receptor for the cytokine macrophage migration inhibitory factor (MIF) which, when bound to the encoded protein, initiates survival pathways and cell proliferation. CD74 protein also interacts with amyloid precursor protein (APP) and suppresses the production of amyloid beta (Abeta). In addition, CD74 protein plays a critical role in MHC class II antigen processing by stabilizing peptide-free class II alpha/beta heterodimers in a complex soon after their synthesis and directing transport of the complex from the endoplasmic reticulum to the endosomal/lysosomal system where the antigen processing and binding of antigenic peptides to MHC class II takes place. CD74 protein serves as cell surface receptor for the cytokine MIF. Diseases associated with CD74 include undifferentiated pleomorphic sarcoma and mantle cell lymphoma. Among its related pathways are response to elevated platelet cytosolic Ca²⁺ and innate immune system. In some embodiments, the CD74 gene, located on chromosome 5q in humans, consists of 9 exons. Orthologs are known from chimpanzee, rhesus monkey, dog, mouse, rat, chicken, and frog. Knockout mouse lines, including CD74^{tm1Doi} (Viville et al. (1993) Cell 72:635-648), CD74^{tm1Liz} (Bikoff et al. (1993) JExp Aed 177:1699-1712), CD74^{tm1Eae} (Elliott et al. (1994) J Exp Med 179:681-694), and CD74^{tm1Anjm}(Barlow et al. (2010) Nat Afed 16:59-66), and CD74^{tm2Liz} (Takaesu et al. (1995) Immunrity 3:385-396), exist. In some embodiments, human CD74 protein has 296 amino acids and/or a molecular mass of 33516 Da. In some embodiments, CD74 protein contains a MHC2-interacting domain, a class II MHC-associated invariant chain trimerisation domain, and thyroglobulin type I repeats.

The term “CD74” is intended to include fragments, variants (e.g., allelic variants), and derivatives thereof. Representative human CD74 cDNA and human CD74 protein sequences are well-known in the art and are publicly available from the National Center for Biotechnology Information (NCBI) (see, for example, ncbi.nlm.nih.gov/gene/972). For example, at least three different human CD74 isoforms are known. Human CD74 isoform A (NP_001020330.1) is encodable by the transcript variant 1 (NM_001025159.2), which is the longest transcript. Human CD74 isoform B (NP_004346.1) is encodable by the transcript variant 2 (NM_004355.3), which lacks lacks an in-frame exon in the 3′ coding region, compared to variant 1. Human CD74 isoform C (NP_001020329.1) is encodable by the transcript variant 3 (NM_001025158.2), which lacks three consecutive exons in the 3′ coding region, which results in a frame-shift, compared to variant 1. Nucleic acid and polypeptide sequences of CD74 orthologs in organisms other than humans are well-known and include, for example, chimpanzee CD74 (NM_001144836.1 and NP_001138308.1), rhesus monkey CD74 (XM_015141237.1 and XP_014996723.1, and XM_015141236.1 and XP_014996722.1), dog CD74 (XM_536468.7 and XP_536468.5; and XM_005619298.3 and XP_005619355.1), mouse CD74 (NM_001042605.1 and NP_001036070.1; and NM_010545.3 and NP_034675.1), rat CD74 (NM_013069.2 and NP_037201.1), chicken CD74 (XM_015293754.2 and XP_015149240.1), and frog CD74 (NM_001197110.1 and NP_001184039.1). Representative sequences of CD74 orthologs are presented below in Table 1.

Anti-CD74 antibodies suitable for detecting CD74 protein are well-known in the art and include, for example, antibodies AF3590 and MAB35901 (R&D systems, Minneapolis, Minn.), antibodies NBP2-29465, NBP2-66762, NBP1-33109, and NBP1-85225 (Novus Biologicals, Littleton, Colo.), antibodies ab9514, ab22603, and ab108393 (AbCam, Cambridge, Mass.), antibodies Cat #: CF507339 and TA507339 (Origene, Rockville, Md.), etc. Other anti-CD74 antibodies are also known and include, for example, those described in U.S. Pat. Pubis. US20140030273, US20170173151, U.S. Pat. No. 7,312,318, and US20170253656. In addition, reagents are well-known for detecting CD74 expression. Multiple clinical tests of CD74 are available in NIH Genetic Testing Registry (GTR®)(e.g., GTR Test ID: GTR000532717.2, offered by Fulgent Clinical Diagnostics Lab (Temple City, Calif.)). Moreover, multiple siRNA, shRNA, CRISPR constructs for reducing CD74 expression can be found in the commercial product lists of the above-referenced companies, such as siRNA product #SR300649, shRNA products #TR314068, TL314068, TG314068, and CRISPR products #KN205824 from Origene Technologies (Rockville, Md.), CRISPR gRNA products from Applied Biological Materials (K6656308) and from Santa Cruz (sc-400279), and RNAi products from Santa Cruz (Cat #sc-35023 and sc-42802). It is to be noted that the term can further be used to refer to any combination of features described herein regarding CD74 molecules. For example, any combination of sequence composition, percentage identify, sequence length, domain structure, functional activity, etc. can be used to describe a CD74 molecule encompassed by the present invention.

The term “CD207” refers to CD207. CD207 protein is expressed only in Langerhans cells which are immature dendritic cells of the epidermis and mucosa. It is localized in the Birbeck granules, organelles present in the cytoplasm of Langerhans cells and consisting of superimposed and zippered membranes. It is a C-type lectin with mannose binding specificity, and it has been proposed that mannose binding by CD207 protein leads to internalization of antigen into Birbeck granules and providing access to a nonclassical antigen-processing pathway. Mutations in CD207 result in Birbeck granules deficiency or loss of sugar binding activity. In addition, CD207 protein is a calcium-dependent lectin displaying mannose-binding specificity. CD207 protein induces the formation of Birbeck granules (BGs) and is a potent regulator of membrane superimposition and zippering. CD207 protein binds to sulfated as well as mannosylated glycans, keratan sulfate (KS) and beta-glucans, facilitates uptake of antigens, and is involved in the routing and/or processing of antigen for presentation to T cells. CD207 is a major receptor on primary Langerhans cells for Candida species, Saccharomyces species, and Malassezia furfur. CD207 protects against human immunodeficiency virus-1 (HIV-1) infection. It binds to high-mannose structures present on the envelope glycoprotein which is followed by subsequent targeting of the virus to the Birbeck granules leading to its rapid degradation. Diseases associated with CD207 include birbeck granule deficiency and langerhans cell histiocytosis. Among its related pathways are the innate immune system and class I MHC-mediated antigen processing and presentation. In some embodiments, the CD207 gene, located on chromosome 2p in humans, consists of 10 exons. Orthologs are known from chimpanzee, rhesus monkey, cow, mouse, rat, and frog. Knockout mouse lines, including CD207^{tm1Mal} (Kissenpfennig et al. (2005)Mol Cell boil 25:88-99) and CD207^{tm1.Cfg} (Orr et al. (2013) Glycobiology 23:363-380), exist. In some embodiments, human CD207 protein has 328 amino acids and/or a molecular mass of 36725 Da. In some embodiments, CD207 protein contains a Rad50 zinc hook motif and a C-type lectin-like domain. The C-type lectin domain mediates dual recognition of both sulfated and mannosylated glycans.

The term “CD207” is intended to include fragments, variants (e.g., allelic variants), and derivatives thereof. Representative human CD207 cDNA and human CD207 protein sequences are well-known in the art and are publicly available from the National Center for Biotechnology Information (NCBI) (see, for example, ncbi.nlm.nih.gov/gene/50489). For example, human CD207 (NP_056532.4) is encodable by the transcript (NM_015717.4). Nucleic acid and polypeptide sequences of CD207 orthologs in organisms other than humans are well-known and include, for example, chimpanzee CD207 (XM_016945490.2 and XP_016800979.1), rhesus monkey CD207 (XM_001100466.3 and XP_001100466.2), cattle CD207 (XM_015473414.2 and XP_015328900.2), and mouse CD207 (NM_144943.3 and NP_659192.2), rat CD207 (NM_013069.2 and NP_037201.1). Representative sequences of CD207 orthologs are presented below in Table 1.

Anti-CD207 antibodies suitable for detecting CD207 protein are well-known in the art and include, for example, antibodies AF2088, BAF2088, and MAB2088 (R&D systems, Minneapolis, Minn.), antibodies DDX0362P-100, DDX0363P-100, DDX0361P-100, and NB100-56733 (Novus Biologicals, Littleton, Colo.), antibodies ab192027 (AbCam, Cambridge, Mass.), antibodies Cat #: TA336470 and TA349377 (Origene, Rockville, Md.), etc. In addition, reagents are well-known for detecting CD207 expression. Multiple clinical tests of CD207 are available in NIH Genetic Testing Registry (GTR®) (e.g., GTR Test ID: GTR000516372.2, offered by Fulgent Clinical Diagnostics Lab (Temple City, Calif.)). Moreover, multiple siRNA, shRNA, CRISPR constructs for reducing CD207 expression can be found in the commercial product lists of the above-referenced companies, such as siRNA product #SR309386, shRNA products #TL305520V, TR305520, TG305520, TF305520, TL305520 and CRISPR products #KN204669 from Origene Technologies (Rockville, Md.), CRISPR gRNA products from Applied Biological Materials (K4909208) and from Santa Cruz (sc-401949), and RNAi products from Santa Cruz (Cat #sc-43888 and sc-43889). It is to be noted that the term can further be used to refer to any combination of features described herein regarding CD207 molecules. For example, any combination of sequence composition, percentage identify, sequence length, domain structure, functional activity, etc. can be used to describe a CD207 molecule encompassed by the present invention.

The term “LRRC25” refers to Leucine Rich Repeat Containing 25. LRRC25 gene has a broad expression in tissues including spleen and bone marrow. LRRC25 protein may be involved in the activation of cells of innate and acquired immunity. It is downregulated in CD40-activated monocyte-derived dendritic cells. Diseases associated with LRRC25 include transient global amnesia. In some embodiments, the LRRC25 gene, located on chromosome 19p in humans, consists of 3 exons. Orthologs are known from chimpanzee, rhesus monkey, dog, cow, mouse, and rat. In some embodiments, human LRRC25 protein has 305 amino acids and/or a molecular mass of 33179 Da. In some embodiments, LRRC25 protein contains two copies of leucine rich repeat, and a GRB2-binding adapter.

The term “LRRC25” is intended to include fragments, variants (e.g., allelic variants), and derivatives thereof. Representative human LRRC25 cDNA and human LRRC25 protein sequences are well-known in the art and are publicly available from the National Center for Biotechnology Information (NCBI) (see, for example, ncbi.nlm.nih.gov/gene/126364). For example, human LRRC25 (NP_660299.2) is encodable by the transcript (NM_145256.2). Nucleic acid and polypeptide sequences of LRRC25 orthologs in organisms other than humans are well-known and include, for example, chimpanzee LRRC25 (XM_009435028.3 and XP_009433303.1; and XM_001173930.6 and XP_001173930.1), rhesus monkey LRRC25 (XM_001114428.3 and XP_001114428.1), dog LRRC25 (XM_847238.5 and XP_852331.3; and XM_014122405.2 and XP_013977880.1), cattle LRRC25 (XM_005208421.4 and XP_005208478.1), mouse LRRC25 (NM_153074.3 and NP_694714.1), and rat LRRC25 (XM_573882.6 and XP_573882.1; XM_006252977.3 and XP_006253039.1; XM_008771187.2 and XP_008769409.1; XM_006252978.3 and XP_006253040.1; and XM_008771188.2 and XP_008769410.1). Representative sequences of LRRC25 orthologs are presented below in Table 1.

Anti-LRRC25 antibodies suitable for detecting LRRC25 protein are well-known in the art and include, for example, antibody GTX45692 (GeneTex, Irvine, Calif.), antibody sc-514216 (Santa Cruz Biotechnology), antibodies NBP2-03747, NBPI-83476, and NBP2-45673 (Novus Biologicals, Littleton, Colo.), antibody ab84954 (AbCam, Cambridge, Mass.), antibodies Cat #: TA504941 and CF504941 (Origene, Rockville, Md.), etc. In addition, reagents are well-known for detecting LRRC25 expression. Multiple clinical tests of LRRC25 are available in NIH Genetic Testing Registry (GTR®) (e.g., GTR Test ID: GTR000541158.2, offered by Fulgent Clinical Diagnostics Lab (Temple City, Calif.)). Moreover, multiple siRNA, shRNA, CRISPR constructs for reducing LRRC25 expression can be found in the commercial product lists of the above-referenced companies, such as siRNA product #SR325688, shRNA products #TL303467, TR303467, TG303467, TF303467, TL303467V and CRISPR products #KN209911 from Origene Technologies (Rockville, Md.), CRISPR gRNA products from Applied Biological Materials (K3598208) and from Santa Cruz (sc-414270), and RNAi products from Santa Cruz (Cat #sc-97675 and sc-149064). It is to be noted that the term can further be used to refer to any combination of features described herein regarding LRRC25 molecules. For example, any combination of sequence composition, percentage identify, sequence length, domain structure, functional activity, etc. can be used to describe a LRRC25 molecule encompassed by the present invention.

The term “SELPLG” or “PSGL 1” refers to Selectin P Ligand, a glycoprotein that functions as a high affinity counter-receptor for the cell adhesion molecules P-, E- and L-selectin expressed on myeloid cells and stimulated T lymphocytes. As such, SELPLG protein plays a critical role in leukocyte trafficking during inflammation by tethering of leukocytes to activated platelets or endothelia expressing selectins. SELPLG protein has two post-translational modifications, tyrosine sulfation and the addition of the sialyl Lewis x tetrasaccharide (sLex) to its O-linked glycans, for its high-affinity binding activity.

Aberrant expression of SELPLG and polymorphisms in SELPLG are associated with defects in the innate and adaptive immune response. SELPLG is a SLe(x)-type proteoglycan, which through high affinity, calcium-dependent interactions with E-, P- and L-selectins, mediates rapid rolling of leukocytes over vascular surfaces during the initial steps in inflammation. SELPLG is critical for initial leukocyte capture. In some embodiments, the SELPLG gene, located on chromosome 12q in humans, consists of 3 exons. Orthologs are known from chimpanzee, rhesus monkey, dog, cow, mouse, and rat. Knockout mouse lines, including Selpg^{tm2Rpmc} (Miner et al. (2008) Blood 112:2035-2045), Selpg^{tm1Fur} (Yang et al. (1999) Jap Med 190:1769-1782), and Selplg^{tm1Rpmc} (Xia et al. (2002) J Clin Invest 109:939-950), exist. In some embodiments, human SELPLG protein has 412 amino acids and/or a molecular mass of 43201 Da. In some embodiments, SELPLG protein contains a ribonuclease E/G family domain and/or can act as a receptor for enterovirus 71 during microbial infection. The known binding partners of SELPLG include, e.g., P-, E- and L-selectins, SNX20, MSN and SYK.

The term “SELPLG” is intended to include fragments, variants (e.g., allelic variants), and derivatives thereof. Representative human SELPLG cDNA and human SELPLG protein sequences are well-known in the art and are publicly available from the National Center for Biotechnology Information (NCBI) (see, for example, ncbi.nlm.nih.gov/gene/6404). For example, at least two different human SELPLG isoforms are known. Human SELPLG isoform 1 (NP_001193538.1) is encodable by the transcript variant 1 (NM_001206609.1), which is the longer transcript. Human SELPLG isoform 2 (NP_002997.2) is encodable by the transcript variant 2 (NM_003006.4), which differs in the 5′ UTR, lacks a portion of the 5′ coding region, and initiates translation at a downstream start codon compared to variant 1. The encoded isoform 2 has a shorter N-terminus, compared to isoform 1. Nucleic acid and polypeptide sequences of SELPLG orthologs in organisms other than humans are well-known and include, for example, chimpanzee SELPLG (XM_016924121.2 and XP_016779610.1), rhesus monkey SELPLG (XM_015152715.1 and XP_015008201.1; and XM_015152716.1 and XP_015008202.1), dog SELPLG (NM_001242719.1 and NP_001229648.1), cattle SELPLG (NM_001037628.2 and NP_001032717.2; and NM_001271160.1 and NP_001258089.1), mouse SELPLG (NM_009151.3 and NP_033177.3), and rat SELPLG (NM_001013230.1 and NP_001013248.1). Representative sequences of SELPLG orthologs are presented below in Table 1.

Anti-SELPLG antibodies suitable for detecting SELPLG protein are well-known in the art and include, for example, antibodies GTX19793, GTX54688, and GTX34468 (GeneTex, Irvine, Calif.), antibodies sc-365506, and sc-398402 (Santa Cruz Biotechnology), antibodies MAB9961, MAB996, NBP2-53344, and AF3345 (Novus Biologicals, Littleton, Colo.), antibodies ab68143, ab66882, and ab110096 (AbCam, Cambridge, Mass.), antibodies Cat #: TA349432 and TA338245 (Origene, Rockville, Md.), etc. Other anti-SELPLG antibodies are also known and include, for example, those described in U.S. Pat. Publs. US20130209449, US20170190782A1, and US20070160601A, and U.S. Pat. Nos. U.S. Pat. No. 7,833,530B2 and U.S. Pat. No. 9,487,585B2. In addition, reagents are well-known for detecting SELPLG expression. Multiple clinical tests of SELPLG are available in NIH Genetic Testing Registry (GTR®) (e.g., GTR Test ID: GTR000547735.2, offered by Fulgent Clinical Diagnostics Lab (Temple City, Calif.)). Moreover, multiple siRNA, shRNA, CRISPR constructs for reducing SELPLG expression can be found in the commercial product lists of the above-referenced companies, such as siRNA product #SR321732, shRNA products #TL309563, TR309563, TG309563, TF309563, TL309563V and CRISPR products #KN206507 from Origene Technologies (Rockville, Md.), CRISPR gRNA products from Applied Biological Materials (K6134408) and from Santa Cruz (sc-401534), and RNAi products from Santa Cruz (Cat #sc-36323 and sc-42833). It is to be noted that the term can further be used to refer to any combination of features described herein regarding SELPLG molecules. For example, any combination of sequence composition, percentage identify, sequence length, domain structure, functional activity, etc. can be used to describe a SELPLG molecule encompassed by the present invention.

The term “AIF1” refers to Allograft Inflammatory Factor 1, a protein that binds actin and calcium. AIF1 gene is induced by cytokines and interferon and may promote macrophage activation and growth of vascular smooth muscle cells and T-lymphocytes.

Polymorphisms in AIF1 may be associated with systemic sclerosis. AIF1 is an actin-binding protein that enhances membrane ruffling and RAC activation. It enhances the actin-bundling activity of LCP1, binds calcium, and plays a role in RAC signaling and in phagocytosis. AIF1 promotes the proliferation of vascular smooth muscle cells and of T-lymphocytes, enhances lymphocyte migration, and plays a role in vascular inflammation.

Diseases associated with AIF include chronic inflammatory demyelinating polyneuropathy and acute diarrhea. Among its related pathways are spinal cord injury. In some embodiments, the AIF1 gene, located on chromosome 6p in humans, consists of 6 exons. Knockout mouse lines, including Aif1^{tm1.1(KOMP)Wtsi} (Dickinson et al. (2016) Nature 537:208-514) and Aif1^{tm1Nsib} (Casimiro et al. (2013) Genesis 51:734-740), exist. In some embodiments, human AIF1 protein has 147 amino acids and/or a molecular mass of 16703 Da. In some embodiments, AIF1 protein contains a penta-EF hand (PEF) family domain.

The known binding partners of AIF1 include, e.g., LCP1.

The term “AIF1” is intended to include fragments, variants (e.g., allelic variants), and derivatives thereof. Representative human AIF1 cDNA and human AIF1 protein sequences are well-known in the art and are publicly available from the National Center for Biotechnology Information (NCBI) (see, for example, ncbi.nlm.nih.gov/gene/199). For example, at least two different human AIF1 isoforms are known. Human AIF1 isoform 1 (NP_001305899.1 and NP_116573.1) is encodable by the transcript variant 1 (NM_032955.2) and the transcript variant 4 (NM_001318970.1). Human AIF1 isoform 3 (NP_001614.3) is encodable by the transcript variant 3 (NM_001623.4), which encodes the longest isoform. The transcript variant 1 differs in the 5′ UTR, lacks a portion of the 5′ coding region, and initiates translation at a downstream start codon compared to variant 3. The transcript variant 4 uses an alternate splice site in the 5′ region and initiates translation at a downstream start codon compared to variant 3. Variants 1 and 4 encode the same isoform 1, which has a shorter N-terminus than isoform 3. Nucleic acid and polypeptide sequences of AIF1 orthologs in organisms other than humans are well-known and include, for example, chimpanzee AIF1 (XM_009450914.2 and XP_009449189.2; XM_009450910.2 and XP_009449185.2; XM_001154743.5 and XP_001154743.1; XM_009450908.3 and XP_009449183.1; and XM_024357095.1 and XP_024212863.1), rhesus monkey AIF1 (NM_001047118.1 and NP_001040583.1), dog AIF1 (XM_532072.6 and XP_532072.2), cattle AIF1 (NM_173985.2 and NP_776410.1), mouse AIF1 (NM_001361501.1 and NP_001348430.1; NM_001361502.1 and NP_001348431.1; NM_019467.3 and NP_062340.1), and rat AIF1 (NM_017196.3 and NP_058892.1). Representative sequences of AIF1 orthologs are presented below in Table 1.

Anti-AIF1 antibodies suitable for detecting AIF1 protein are well-known in the art and include, for example, antibodies GTX100042, GTX101495, and GTX632426 (GeneTex, Irvine, Calif.), antibodies sc-32725, and sc-398406 (Santa Cruz Biotechnology), antibodies NB100-1028, NBP2-19019, NBP2-16908, and NB100-2833 (Novus Biologicals, Littleton, Colo.), antibodies ab5076, ab178847, and ab48004 (AbCam, Cambridge, Mass.), antibodies Cat #: AP08793PU-N and AP08912PU-N(Origene, Rockville, Md.), etc. In addition, reagents are well-known for detecting AIF1 expression. Multiple clinical tests of AIF1 are available in NIH Genetic Testing Registry (GTR®) (e.g., GTR Test ID: GTR000542089.2, offered by Fulgent Clinical Diagnostics Lab (Temple City, Calif.)). Moreover, multiple siRNA, shRNA, CRISPR constructs for reducing AIF1 expression can be found in the commercial product lists of the above-referenced companies, such as siRNA product #SR300138, shRNA products #TL314878, TR314878, TG314878, TF314878, TL314878V and CRISPR products #KN203154 from Origene Technologies (Rockville, Md.), CRISPR gRNA products from Applied Biological Materials (K6902508) and from Santa Cruz (sc-400513), and RNAi products from Santa Cruz (Cat #sc-36323 and sc-42833). It is to be noted that the term can further be used to refer to any combination of features described herein regarding AIF1 molecules. For example, any combination of sequence composition, percentage identify, sequence length, domain structure, functional activity, etc. can be used to describe an AIF1 molecule encompassed by the present invention.

The term “CD84” refers to CD84 molecule, a membrane glycoprotein that is a member of the signaling lymphocyte activation molecule (SLAM) family. This family forms a subset of the larger CD2 cell-surface receptor Ig superfamily. The encoded protein is a homophilic adhesion molecule that is expressed in numerous immune cells types and is involved in regulating receptor-mediated signaling in those cells. Diseases associated with CD84 include leukemia, chronic lymphocytic. Among its related pathways are response to elevated platelet cytosolic ca2+ and cell surface interactions at the vascular wall. In some embodiments, the CD84 gene, located on chromosome 1q in humans, consists of 9 exons. Knockout mouse lines, including Cd84^{tm1Beni} (Hofmann et al. (2014) Plos One 9:e115306), Cd84 DlbOimP(Dickinson et al. (2016) Nature 537:508-514), and Cd84^{tm1Pls} (Cannnons et al. (2010) Immunity 32:253-265), exist. In some embodiments, human CD84 protein has 345 amino acids and/or a molecular mass of 38782 Da. In some embodiments, CD84 protein contains a N-terminal immunoglobulin (Ig)-like domain and an immunoglobulin domain. CD84 is a self-ligand receptor of the signaling lymphocytic activation molecule (SLAM) family. SLAM receptors triggered by homo- or heterotypic cell-cell interactions are modulating the activation and differentiation of a wide variety of immune cells and thus are involved in the regulation and interconnection of both innate and adaptive immune response. Activities are controlled by presence or absence of small cytoplasmic adapter proteins, SH2D1A/SAP and/or SH2D1B/EAT-2. CD84 can mediate natural killer (NK) cell cytotoxicity dependent on SH2D1A and SH2D1B. CD84 increases proliferative responses of activated T-cells and SH2D1A/SAP does not seem be required for this process. Homophilic interactions of CD84 enhance interferon gamma/IFNG secretion in lymphocytes and induce platelet stimulation via a SH2DA-dependent pathway. CD84 may serve as a marker for hematopoietic progenitor cells (Martin et al. (2001) J Immunol 167:3668-3676). CD84 is required for a prolonged T-cell:B-cell contact, optimal T follicular helper function, and germinal center formation. In germinal centers, CD84 is involved in maintaining B-cell tolerance and in preventing autoimmunity. In mast cells, CD84 negatively regulates high affinity immunoglobulin epsilon receptor signaling (Alvarez-Errico et al. (2011) J Immunol 187:5577-5586).

The term “CD84” is intended to include fragments, variants (e.g., allelic variants), and derivatives thereof. Representative human CD84 cDNA and human CD84 protein sequences are well-known in the art and are publicly available from the National Center for Biotechnology Information (NCBI) (see, for example, ncbi.nlm.nih.gov/gene/8832). For example, at least five different human CD84 isoforms are known. Human CD84 isoform 1 (NP_001171808.1) is encodable by the transcript variant 1 (NM_001184879.1), which is the longest transcript. Human CD84 isoform 2 (NP_003865.1) is encodable by the transcript variant 2 (NM_003874.3), which lacks an alternate, in-frame segment, compared to variant 1. Human CD84 isoform 3 (NP_001171810.1) is encodable by the transcript variant 3 (NM_001184881.1), which lacks two alternate segments, one of which shifts the reading frame, compared to variant 1. Human CD84 isoform 4 (NP_001171811.1) is encodable by the transcript variant 4 (NM_001184882.1), which lacks two alternate segments, compared to variant 1. Human CD84 isoform 5 (NP_001317671.1) is encodable by the transcript variant 5 (NM_001330742.1), which uses an alternate in-frame splice junction compared to variant 1. Nucleic acid and polypeptide sequences of CD84 orthologs in organisms other than humans are well-known and include, for example, chimpanzee CD84 (XM_016930506.2 and XP_016785995.1; and XM_001172059.4 and XP_001172059.1), rhesus monkey CD84 (XM_001117595.3 and XP_001117595.1, XM_015113569.1 and XP_014969055.1, and XM_015113561.1 and XP_014969047.1), dog CD84 (XM_022415343.1 and XP_022271051.1; and XM_005640884.3 and XP_005640941), cattle CD84 (XM_024989885.1 and XP_024845653.1; XM_024989884.1 and XP_024845652.1; XM_010802802.3 and XP_010801104.1; XM_010802805.3 and XP_010801107.1; XM_024989882.1 and XP_024845650.1, XM_024989883.1 and XP_024845651.1; and XM_024989886.1 and XP_024845654.1), mouse CD84 (NM_013489.3 and NP_038517.1; NM_001252472.1 and NP_001239401.1; and NM_001289470.1 and NP_001276399.1), and rat CD84 (NM_001192006.1 and NP_001178935.1). Representative sequences of CD84 orthologs are presented below in Table 1 and Table 2 because, as demonstrated herein, CD84 can differentially affect monocytes and/or macrophages to be more pro-inflammatory or more anti-inflammatory depending upon the context.

Anti-CD84 antibodies suitable for detecting CD84 protein are well-known in the art and include, for example, antibodies GTX32506, GTX75849, and GTX75851 (GeneTex, Irvine, Calif.), antibodies sc-39821, and sc-70810 (Santa Cruz Biotechnology), antibodies MAB1855, AF1855, NBP2-49635, and NB100-65929 (Novus Biologicals, Littleton, Colo.), antibodies ab131256, ab202841, and ab176513 (AbCam, Cambridge, Mass.), antibodies Cat #: SM1845R and SM1845PT (Origene, Rockville, Md.), etc. Other anti-CD84 antibodies are also known and include, for example, those described in U.S. Pat. Publs.

US20140147451A1, US20170260270A1, and US20180327493. In addition, reagents are well-known for detecting CD84 expression. Multiple clinical tests of CD84 are available in NIH Genetic Testing Registry (GTR®) (e.g, GTR Test ID: GTR000532250.2, offered by Fulgent Clinical Diagnostics Lab (Temple City, Calif.)). Moreover, multiple siRNA, shRNA, CRISPR constructs for reducing CD84 expression can be found in the commercial product lists of the above-referenced companies, such as siRNA product #SR322568, shRNA products #TL314062, TR314062, TG314062, TF314062, TL314062V and CRISPR products #KN204477 from Origene Technologies (Rockville, Md.), CRISPR gRNA products from Applied Biological Materials (K6196808) and from Santa Cruz (sc-416482), and RNAi products from Santa Cruz (Cat #sc-42810 and sc-42811). It is to be noted that the term can further be used to refer to any combination of features described herein regarding CD84 molecules. For example, any combination of sequence composition, percentage identify, sequence length, domain structure, functional activity, etc. can be used to describe a CD84 molecule encompassed by the present invention.

The term “IGSF6” refers to Immunoglobulin Superfamily Member 6. Diseases associated with IGSF6 include dysbaric osteonecrosis and inflammatory bowel disease. In some embodiments, the IGSF6 gene, located on chromosome 16p in humans, consists of 6 exons. IGSF6 is coded entirely within the intron of METTL9 which is transcribed in the opposite strand of the DNA. IGSF6 is localized to a locus associated with inflammatory bowel disease. In some embodiments, human IGSF6 protein has 241 amino acids and/or a molecular mass of 27013 Da. In some embodiments, IGSF6 contains an immunoglobulin domain.

The term “IGSF6” is intended to include fragments, variants (e.g., allelic variants), and derivatives thereof. Representative human IGSF6 cDNA and human IGSF6 protein sequences are well-known in the art and are publicly available from the National Center for Biotechnology Information (NCBI) (see, for example, ncbi.nlm.nih.gov/gene/10261). For example, human IGSF6 (NP_005840.2) is encodable by the transcript variant 1 (NM_005849.3). Nucleic acid and polypeptide sequences of IGSF6 orthologs in organisms other than humans are well-known and include, for example, chimpanzee IGSF6 (XM_001160217.6 and XP_001160217.1; and XM_016928690.2 and XP_016784179.1), rhesus monkey IGSF6 (XM_001093144.3 and XP_001093144.1), dog IGSF6 (XM_005621426.3 and XP_005621483.1; XM_005621428.3 and XP_005621485.1; and XM_022419960.1 and XP_022275668.1), cattle IGSF6 (XM_002697991.6 and XP_002698037.1), mouse IGSF6 (NM_030691.1 and NP_109616.1), rat IGSF6 (NM_133542.2 and NP_598226.1); and chicken IGSF6 (NM_001277599.1 and NP_001264528.1). Representative sequences of IGSF6 orthologs are presented below in Table 1.

Anti-IGSF6 antibodies suitable for detecting IGSF6 protein are well-known in the art and include, for example, antibody sc-377053 (Santa Cruz Biotechnology), antibodies DDX0220P-100, NBP1-84061, H00010261-M02, and H00010261-M01 (Novus Biologicals, Littleton, Colo.), antibody ab197659 (AbCam, Cambridge, Mass.), antibody Cat #: TA322553 (Origene, Rockville, Md.), etc. In addition, reagents are well-known for detecting IGSF6 expression. Multiple clinical tests of IGSF6 are available in NIH Genetic Testing Registry (GTR®) (e.g., GTR Test ID: GTR000542139.2, offered by Fulgent Clinical Diagnostics Lab (Temple City, Calif.)). Moreover, multiple siRNA, shRNA, CRISPR constructs for reducing IGSF6 expression can be found in the commercial product lists of the above-referenced companies, such as siRNA product #SR323049, shRNA products #TL312209, TR312209, TG312209, TF312209, TL312209V and CRISPR products #KN204717 from Origene Technologies (Rockville, Md.), CRISPR gRNA products from Applied Biological Materials (K7017208) and from Santa Cruz (sc-411445), and RNAi products from Santa Cruz (Cat #sc-93333 and sc-146192). It is to be noted that the term can further be used to refer to any combination of features described herein regarding IGSF6 molecules. For example, any combination of sequence composition, percentage identify, sequence length, domain structure, functional activity, etc. can be used to describe an IGSF6 molecule encompassed by the present invention.

The term “CD48” refers to CD48 molecule, a member of the CD2 subfamily of immunoglobulin-like receptors which includes SLAM (signaling lymphocyte activation molecules) proteins. CD48 protein is found on the surface of lymphocytes and other immune cells, dendritic cells and endothelial cells, and participates in activation and differentiation pathways in these cells. CD48 protein does not have a transmembrane domain, however, but is held at the cell surface by a GPI anchor via a C-terminal domain which maybe cleaved to yield a soluble form of the receptor. Among its related pathways are response to elevated platelet cytosolic Ca2+ and hematopoietic stem cell differentiation pathways and lineage-specific markers. In some embodiments, the CD48 gene, located on chromosome 1q in humans, consists of 5 exons. In some embodiments, human CD48 protein has 243 amino acids and/or a molecular mass of 27683 Da. A knockout mouse line, called CD48^{tm1Rsr} (Gonazalez-Cabrero et al. (1999) Proc Natl Acad Sci 96:1019-1023), exists. CD48 interacts with CD244 in a heterophilic manner. In some embodiments, CD48 protein contains one or more immunoglobulin-like domains.

The term “CD48” is intended to include fragments, variants (e.g., allelic variants), and derivatives thereof. Representative human CD48 cDNA and human CD48 protein sequences are well-known in the art and are publicly available from the National Center for Biotechnology Information (NCBI) (see, for example, ncbi.nlm.nih.gov/gene/962). For example, at least two different human CD48 isoforms are known. Human CD48 isoform 1 (NP_001769.2) is encodable by the transcript variant 1 (NM_001778.3), which is the shorter transcript. Human CD48 isoform 2 (NP_001242959.1) is encodable by the transcript variant 2 (NM_001256030.1), which differs in the 3′ UTR and coding region compared to variant 1. The encoded isoform 2 is longer and has a distinct C-terminus compared to isoform 1. Nucleic acid and polypeptide sequences of CD48 orthologs in organisms other than humans are well-known and include, for example, chimpanzee CD48 (XM_009435717.1 and XP_009433992.1; and XM_001172145.3 and XP_001172145.2), rhesus monkey CD48 (XM_015113628.1 and XP_014969114.1; XM_015113634.1 and XP_014969120.1; and XM_015113619.1 and XP_014969105.1), dog CD48 (XM_545759.6 and XP_545759.2; and XM_022415374.1 and XP_022271082.1), cattle CD48 (NM_001046002.1 and NP_001039467.1), mouse CD48 (NM_007649.5 and NP_031675.1: and NM_001360767.1 and NP_001347696.1), rat CD48 (NM_139103.1 and NP_620803.1); and chicken CD48 (NM_001277599.1 and NP_001264528.1). Representative sequences of CD48 orthologs are presented below in Table 1 and Table 2 because, as demonstrated herein, CD48 can differentially affect monocytes and/or macrophages to be more pro-inflammatory or more anti-inflammatory depending upon the context Anti-CD48 antibodies suitable for detecting CD48 protein are well-known in the art and include, for example, antibodies sc-70719, sc-70718 (Santa Cruz Biotechnology), antibodies AF3327, AF3644, MAB36441, and MAB-3644 (Novus Biologicals, Littleton, Colo.), antibodies ab9185, ab134049, ab119873, and ab76904 (AbCam, Cambridge, Mass.), antibodies Cat #: TA351055, TA320283 (Origene, Rockville, Md.), etc. Other anti-CD48 antibodies are also known and include, for example, those described in U.S. Pat. No.

U.S. Pat. No. 9,097,717B2 and U.S. Pat. Pubis. US20120076790, US20130230533, and US20180092984. In addition, reagents are well-known for detecting CD48 expression. Multiple clinical tests of CD48 are available in NIH Genetic Testing Registry (GTR®) (e.g., GTR Test ID: GTR000532164.2, offered by Fulgent Clinical Diagnostics Lab (Temple City, Calif.)). Moreover, multiple siRNA, shRNA, CRISPR constructs for reducing CD48 expression can be found in the commercial product lists of the above-referenced companies, such as siRNA product #SR300685, shRNA products #TL314079, TR314079, TG314079, TF314079, TL314079V and CRISPR products #KN204849 from Origene Technologies (Rockville, Md.), CRISPR gRNA products from Applied Biological Materials (K7408008) and from Santa Cruz (sc-416692), and RNAi products from Santa Cruz (Cat #sc-35008 and sc-35009). It is to be noted that the term can further be used to refer to any combination of features described herein regarding CD48 molecules. For example, any combination of sequence composition, percentage identify, sequence length, domain structure, functional activity, etc. can be used to describe a CD48 molecule encompassed by the present invention.

The term “CD33” refers to CD33 molecule, a putative adhesion molecule of myelomonocytic-derived cells that mediates sialic-acid dependent binding to cells. CD33 preferentially binds to alpha-2,6-linked sialic acid. The sialic acid recognition site may be masked by cis interactions with sialic acids on the same cell surface. In the immune response, CD33 may act as an inhibitory receptor upon ligand induced tyrosine phosphorylation by recruiting cytoplasmic phosphatase(s) via their SH2 domain(s) that block signal transduction through dephosphorylation of signaling molecules. CD33 induces apoptosis in acute myeloid leukemia in vitro. Diseases associated with CD33 include gallbladder lymphoma and extracutaneous mastocytoma. Among its related pathways are hematopoietic stem cell differentiation pathways and lineage-specific markers and innate immune system. In some embodiments, the CD33 gene, located on chromosome 19q in humans, consists of 14 exons. In some embodiments, human CD33 protein has 364 amino acids and/or a molecular mass of 39825 Da. CD33 interacts with PTPN6/SHP-1 and PTPN11/SHP-2 upon phosphorylation. In some embodiments, human CD33 protein contains two copies of a cytoplasmic motif that is referred to as the immunoreceptor tyrosine-based inhibitor motif (ITIM). This motif is involved in modulation of cellular responses. The phosphorylated ITIM motif can bind the SH2 domain of several SH2-containing phosphatases.

The term “CD33” is intended to include fragments, variants (e.g., allelic variants), and derivatives thereof. Representative human CD33 cDNA and human CD33 protein sequences are well-known in the art and are publicly available from the National Center for Biotechnology Information (NCBI) (see, for example, ncbi.nlm.nih.gov/gene/945). For example, at least three different human CD33 isoforms are known. Human CD33 isoform 1 (NP_001763.3) is encodable by the transcript variant 1 (NM_001772.3), which is the longest transcript. Human CD33 isoform 2 (NP_001076087.1) is encodable by the transcript variant 2 (NM_001082618.1), which lacks an alternate in-frame exon in the 5′ coding region, compared to variant 1, resulting in a shorter protein (isoform 2, also known as CD33m), compared to isoform 1. Human CD33 isoform 3 (NP_001171079.1) is encodable by the transcript variant 3 (NM_001177608.1), which differs in the 3′ UTR and coding sequence compared to variant 1. The encoded isoform 3 has a shorter and distinct C-terminus compared to isoform 1. Nucleic acid and polypeptide sequences of CD33 orthologs in organisms other than humans are well-known and include, for example, chimpanzee CD33 (XM_512850.7 and XP_512850.3; XM_009436143.3 and XP_009434418.1; and XM_016936702.2 and XP_016792191.1), rhesus monkey CD33 (XM_015124693.1 and XP_014980179.1; and XM_001114616.3 and XP_001114616.2), and dog CD33 (XM_005616249.2 and XP_005616306.1). Representative sequences of CD33 orthologs are presented below in Table 1.

Anti-CD33 antibodies suitable for detecting CD33 protein are well-known in the art and include, for example, antibodies sc-514119, sc-376184 (Santa Cruz Biotechnology), antibodies NBP2-22377, NBP2-29619, NBP2-37388, and MAB1137 (Novus Biologicals, Littleton, Colo.), antibodies ab199432, ab134115, ab30371, and ab11032 (AbCam, Cambridge, Mass.), antibodies Cat #: CF806758, TA806758 (Origene, Rockville, Md.), etc. Other anti-CD33 antibodies are also known and include, for example, those described in U.S. Pat. Publs. US20150125447A1, US20160362490A1, US20170002074A1, and US20190002560A1 and U.S. Pat. Nos. 7,022,500B1, and 9,587,019B2. In addition, reagents are well-known for detecting CD33 expression. Multiple clinical tests of CD33 are available in NIH Genetic Testing Registry (GTR®) (e.g., GTR Test ID: GTR000532386.2, offered by Fulgent Clinical Diagnostics Lab (Temple City, Calif.)). Moreover, multiple siRNA, shRNA, CRISPR constructs for reducing CD33 expression can be found in the commercial product lists of the above-referenced companies, such as siRNA product #SR319607, shRNA products #TL314092, TR314092, TG314092, TF314092, TL314092V and CRISPR products #KN207023 from Origene Technologies (Rockville, Md.), CRISPR gRNA products from Applied Biological Materials (K3368408) and from Santa Cruz (sc-401011), and RNAi products from Santa Cruz (Cat #sc-42782 and sc-42783). It is to be noted that the term can further be used to refer to any combination of features described herein regarding CD33 molecules. For example, any combination of sequence composition, percentage identify, sequence length, domain structure, functional activity, etc. can be used to describe a CD33 molecule encompassed by the present invention.

The term “LST1” refers to Leukocyte Specific Transcript 1, a membrane protein that can inhibit the proliferation of lymphocytes. Expression of LST1 is enhanced by lipopolysaccharide, interferon-gamma, and bacteria. LST1 induces morphological changes including production of filopodia and microspikes when overexpressed in a variety of cell types and may be involved in dendritic cell maturation. Isoform 1 and isoform 2 of LST1 have an inhibitory effect on lymphocyte proliferation. In some embodiments, the LST1 gene, located on chromosome 6p in humans, consists of 6 exons. In some embodiments, human LST1 protein has 97 amino acids and/or a molecular mass of 10792 Da.

The term “LST1” is intended to include fragments, variants (e.g., allelic variants), and derivatives thereof. Representative human LST1 cDNA and human LST1 protein sequences are well-known in the art and are publicly available from the National Center for Biotechnology Information (NCBI) (see, for example, ncbi.nlm.nih.gov/gene/7940). For example, at least six different human LST1 isoforms are known. Human LST1 isoform 1 (NP_009092.3) is encodable by the transcript variant 1 (NM_007161.3), which is the longest transcript. Human LST1 isoform 2 (NP_995309.2) is encodable by the transcript variant 2 (NM_205837.2), which includes an additional exon in the 5′ UTR and lacks an internal exon that causes a frameshift in the 3′ coding region, compared to variant 1. Human LST1 isoform 3 (NP_995310.2) is encodable by the transcript variant 3 (NM_205838.2), which includes an additional exon in the 5′ UTR, lacks an alternate in-frame exon in the 5′ coding region, and uses an alternate in-frame splice site in the 3′ coding region, compared to variant 1. Human LST1 isoform 4 (NP_995311.2) is encodable by the transcript variant 4 (NM_205839.2), includes an additional exon in the 5′ UTR and uses an alternate in-frame splice site in the 3′ coding region, compared to variant 1. Human LST1 isoform 5 (NP_995312.2) is encodable by the transcript variant 5 (NM_205840.2), which lacks an alternate exon in the central coding region and uses an alternate splice site that causes a frameshift in the 3′ coding region compared to variant 1. Human LST1 isoform 6 (NP_001160010.1) is encodable by the transcript variant 6 (NM_001166538.1), which lacks an alternate in-frame exon in the 5′ coding region, compared to variant 1, resulting in an isoform 6 that is shorter than isoform 1. Nucleic acid and polypeptide sequences of LST1 orthologs in organisms other than humans are well-known and include, for example, chimpanzee LST1 (XM_009450906.3 and XP_009449181.1; XM_009450900.3 and XP_009449175.1; XM_009450905.3 and XP_009449180.1; XM_003950777.4 and XP_003950826.1; XM_016955125.2 and XP_016810614.1; XM_016955127.2 and XP_016810616.1; XM_016955126.2 and XP_016810615.1; XM_016955129.2 and XP_016810618.1; XM_009450901.3 and XP_009449176.1; and XM_009450902.3 and XP_009449177.1). Representative sequences of LST1 orthologs are presented below in Table 1.

Anti-LST1 antibodies suitable for detecting LST1 protein are well-known in the art and include, for example, antibody GTX16300 (GeneTex), antibodies NBP1-45072, NBP1-98482 and H00007940-BOIP (Novus Biologicals, Littleton, Colo.), antibodies ab14557 and ab172244 (AbCam, Cambridge, Mass.), antibody Cat #: AM20987PU-N(Origene, Rockville, Md.), etc. In addition, reagents are well-known for detecting LST expression. Multiple clinical tests of LST1 are available in NIH Genetic Testing Registry (GTR®) (e.g., GTR Test ID: GTR000541902.2, offered by Fulgent Clinical Diagnostics Lab (Temple City, Calif.)). Moreover, multiple siRNA, shRNA, CRISPR constructs for reducing LST1 expression can be found in the commercial product lists of the above-referenced companies, such as siRNA product #SR305318, shRNA products #TL311652, TR311652, TG311652, TF311652, TL311652V and CRISPR products #KN213273 from Origene Technologies (Rockville, Md.), CRISPR gRNA products from Applied Biological Materials (K7098808) and from Santa Cruz (sc-407477), and RNAi products from Santa Cruz (Cat #sc-95628 and sc-149136). It is to be noted that the term can further be used to refer to any combination of features described herein regarding LST1 molecules. For example, any combination of sequence composition, percentage identify, sequence length, domain structure, functional activity, etc. can be used to describe a LST molecule encompassed by the present invention.

The term “TNFAIP8L2” or “TIPE2” refers to TNF Alpha Induced Protein 8 Like 2. Diseases associated with TNFAIP8L2 include skin squamous cell carcinoma. Among its related pathways are metabolism and glycerophospholipid biosynthesis. TNFAIP8L2 acts as a negative regulator of innate and adaptive immunity by maintaining immune homeostasis. TNFAIP8L2 acts as a negative regulator of Toll-like receptor and T-cell receptor function. It also prevents hyperresponsiveness of the immune system and maintains immune homeostasis. TNFAIP8L2 inhibits JUN/API and NF-kappa-B activation and promotes Fas-induced apoptosis. In some embodiments, the TNFAIP8L2 gene, located on chromosome 1q in humans, consists of 14 exons. A knockout mouse line, called Tnfaip812^{tm1Yhen}, exists (Sun et al. (2008) Cell 132:415-426). In some embodiments, human TNFAIP8L2 protein has 184 amino acids and/or a molecular mass of 20556 Da. The central region of TNFAIP8L2 protein was initially thought to constitute a DED (death effector) domain. However, 3D-structure data reveal a previously uncharacterized fold that is different from the predicted fold of a DED (death effector) domain. TNFAIP8L2 consists of a large, hydrophobic central cavity that is poised for cofactor binding.

The term “TNFAIP8L2” or “TIPE2” is intended to include fragments, variants (e.g., allelic variants), and derivatives thereof. Representative human TNFAIP8L2 cDNA and human TNFAIP8L2 protein sequences are well-known in the art and are publicly available from the National Center for Biotechnology Information (NCBI) (see, for example, ncbi.nlm.nih.gov/gene/79626). For example, human TNFAIP8L2 (NP_078851.2) is encoded by the transcript (NM_024575.4). Nucleic acid and polypeptide sequences of TNFAIP8L2 orthologs in organisms other than humans are well-known and include, for example, chimpanzee TNFAIP8L2 (XM_009431068.3 and XP_009429343.1; and XM_003308373.4 and XP_003308421.1), rhesus monkey TNFAIP8L2 (NM_001257419.1 and NP_001244348.1), dog TNFAIP8L2 (XM_005630793.3 and XP_005630850.1; and XM_540310.6 and XP_540310.2), cattle TNFAIP8L2 (NM_001034389.1 and NP_001029561.1), mouse TNFAIP8L2 (NM_027206.2 and NP_081482.1), rat TNFAIP8L2 (NM_001014039.1 and NP_001014061.1); tropical clawed frog TNFAIP8L2 (XM_012969840.1 and XP_012825294.1; XM_012969842.1 and XP_012825296.1; XM_012969839.1 and XP_012825293.1; and XM_012969841.1 and XP_012825295.1); and zebrafish TNFAIP8L2 (NM_200374.1 and NP_956668.1). Representative sequences of TNFAIP8L2 orthologs are presented below in Table 1.

Anti-TNFAIP8L2 antibodies suitable for detecting TNFAIP8L2 protein are well-known in the art and include, for example, antibodies H00079626-B01P and H00079626-D01P (Novus Biologicals, Littleton, Colo.), antibodies Cat #: TA315795, AP54305PU-N (Origene, Rockville, Md.), etc. In addition, reagents are well-known for detecting TNFAIP8L2 expression. Multiple clinical tests of TNFAIP8L2 are available in NIH Genetic Testing Registry (GTR®) (e.g., GTR Test ID: GTR000544194.2, offered by Fulgent Clinical Diagnostics Lab (Temple City, Calif.)). Moreover, multiple siRNA, shRNA, CRISPR constructs for reducing TNFAIP8L2 expression can be found in the commercial product lists of the above-referenced companies, such as siRNA product #SR312471, shRNA products #TL300917, TR300917, TG300917, TF300917, TL300917V and CRISPR products #KN209504 from Origene Technologies (Rockville, Md.), CRISPR gRNA products from Applied Biological Materials (K6597108), and RNAi products from Santa Cruz (Cat #sc-76702 and sc-76702-PR). It is to be noted that the term can further be used to refer to any combination of features described herein regarding TNFAIP8L2 molecules. For example, any combination of sequence composition, percentage identify, sequence length, domain structure, functional activity, etc. can be used to describe a TNFAIP8L2 molecule encompassed by the present invention.

The term “SPI1” or “PU.1” refers to Spi-1 Proto-Oncogene, an ETS-domain transcription factor that activates gene expression during myeloid and B-lymphoid cell development. The nuclear protein SPI1 binds to a purine-rich sequence known as the PU-box found near the promoters of target genes, and regulates their expression in coordination with other transcription factors and cofactors. The SPI1 protein can also regulate alternative splicing of target genes. SPI1 binds to the PU-box, a purine-rich DNA sequence (5-GAGGAA-3) that can act as a lymphoid-specific enhancer. SPI1 protein is a transcriptional activator that may be specifically involved in the differentiation or activation of macrophages or B-cells. SPI1 also binds RNA and may modulate pre-mRNA splicing. Diseases associated with SPI1 include inflammatory diarrhea and neutrophil-specific granule deficiency. Among its related pathways are RANK signaling in osteoclasts and osteoclast differentiation. In some embodiments, the SPI1 gene, located on chromosome 11p in humans, consists of 8 exons. Knockout mouse lines, including Spil^{tm1Ram} (McKercher et al. (1996) EMBO J. 15:5647-5658), Spi1^{tm2b(EUCOMM)Wtsi} (International Knockout Mouse Consortium), and Spi1^{tm2.1DgtHuman SPI1} (Iwasaki et al. (2005) Blood 106:1590-1600), exist. In some embodiments, SPI1 protein has 270 amino acids and/or a molecular mass of 31083 Da. SPI1 belongs to ETS family. The known binding partners of SPI1 include, e.g., CEBPD, NONO, RUNX1, SPIB, GFI1, and CEBPE.

The term “SPI1” or “PU.1” is intended to include fragments, variants (e.g., allelic variants), and derivatives thereof. Representative human SPI1 cDNA and human SPI1 protein sequences are well-known in the art and are publicly available from the National Center for Biotechnology Information (NCBI) (see, for example, ncbi.nlm.nih.gov/gene/6688). For example, at least two different human SPI1 isoforms are known. Human SPI1 isoform 1 (NP_001074016.1) is encodable by the transcript variant 1 (NM_001080547.1), which is the longer transcript. Human SPI1 isoform 2 (NP_003111.2) is encodable by the transcript variant 2 (NM_003120.2), which uses an alternate in-frame splice site in the 5′ coding region, compared to variant 1, resulting in a shorter protein (isoform 2). Nucleic acid and polypeptide sequences of SPI1 orthologs in organisms other than humans are well-known and include, for example, dog SPI1 (XM_005631240.3 and XP_005631297.1; and XM_848897.5 and XP_853990.1), cattle SPI1 (NM_001192133.2 and NP_001179062.1), mouse SPI1 (NM_011355.2 and NP_035485.1), rat SPI1 (NM_001005892.2 and NP_001005892.1), chicken SPI1 (NM_205023.1 and NP_990354.1), tropical clawed frog SPI (NM_001145983.1 and NP_001139455.1), and zebrafsh SPI1 (NM_001328368.1 and NP_001315297.1; NM_001328369.1 and NP_001315298.1.; and NM_198062.2 and NP_932328.2). Representative sequences of SPI1 orthologs are presented below in Table 1.

Anti-SPI1 antibodies suitable for detecting SPI1 protein are well-known in the art and include, for example, antibodies GTX128266, GTX101581, and GTX60620 (GeneTex, Irvine, Calif.), antibody sc-390659 (Santa Cruz Biotechnology), antibodies NBP2-27163, NBP1-00135, MAB7124, and MAB5870 (Novus Biologicals, Littleton, Colo.), antibodies ab76543, ab88082, and ab76542 (AbCam, Cambridge, Mass.), antibodies Cat #: CF808850 and TA808850 (Origene, Rockville, Md.), etc. In addition, reagents are well-known for detecting SPI1 expression. Multiple clinical tests of SPI1 are available in NIH Genetic Testing Registry (GTR®) (e.g., GTR Test ID: GTR000546129.2, offered by Fulgent Clinical Diagnostics Lab (Temple City, Calif.)). Moreover, multiple siRNA, shRNA, CRISPR constructs for reducing SPI1 expression can be found in the commercial product lists of the above-referenced companies, such as siRNA product #SR304549, shRNA products #TL316738, TR 316738, TG 316738, TF 316738, TL316738V and CRISPR products #KN212818 from Origene Technologies (Rockville, Md.), CRISPR gRNA products from Applied Biological Materials (K6488408) and from Santa Cruz (sc-400547-KO-2), and RNAi products from Santa Cruz (Cat #sc-36330 and sc36331). It is to be noted that the term can further be used to refer to any combination of features described herein regarding SPI1 molecules. For example, any combination of sequence composition, percentage identify, sequence length, domain structure, functional activity, etc. can be used to describe a SPI molecule encompassed by the present invention.

The term “LILRB2” refers to Leukocyte Immunoglobulin Like Receptor B2, a member of the leukocyte immunoglobulin-like receptor (LIR) family, which is found in humans in a gene cluster at chromosomal region 19q13.4. The encoded protein belongs to the subfamily B class of LIR receptors, generally which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. It is thought to control inflammatory responses and cytotoxicity to help focus the immune response and limit autoreactivity. Among its related pathways are innate immune system and osteoclast differentiation. LILRB2 is a receptor for class I MHC antigens. It recognizes a broad spectrum of HLA-A, HLA-B, HLA-C and HLA-G alleles. LILRB2 is involved in the down-regulation of the immune response and the development of tolerance. LILRB2 competes with CD8A for binding to class I MHC antigens. LILRB2 inhibits FCGRIA-mediated phosphorylation of cellular proteins and mobilization of intracellular calcium ions. In some embodiments, the LILRB2 gene, located on chromosome 19q in humans, consists of 15 exons. In some embodiments, human LILRB2 protein has 598 amino acids and/or a molecular mass of 65039 Da. In some embodiments, LILRB2 contains 3 copies of a cytoplasmic motif that is referred to as the immunoreceptor tyrosine-based inhibitor motif (ITIM). This motif is involved in modulation of cellular responses. The phosphorylated ITIM motif can bind the SH2 domain of several SH2-containing phosphatases. The known binding partners of LILRB2 include, e.g., PTPN6 and FCGRIA.

The term “LILRB2” is intended to include fragments, variants (e.g., allelic variants), and derivatives thereof. Representative human LILRB2 cDNA and human LILRB2 protein sequences are well-known in the art and are publicly available from the National Center for Biotechnology Information (NCBI) (see, for example, ncbi.nlm.nih.gov/gene/10288). For example, at least five different human LILRB2 isoforms are known. Human LILRB2 isoform 1 (NP_005865.3) is encodable by the transcript variant 1 (NM_005874.4), which is the longest transcript. Human LILRB2 isoform 2 (NP_001074447.2 and NP_001265332.2) is encodable by the transcript variant 2 (NM_001080978.3), which uses an alternate in-frame splice site in the central coding region, compared to variant 1, and by the transcript variant 3 (NM_001278403.2), which differs in the 5′ UTR and uses an alternate in-frame splice site in the central coding region, compared to variant 1. The encoded isoform 2 is shorter, compared to isoform 1. Both variants 2 and 3 encode the same isoform. Human LILRB2 isoform 3 (NP_001265333.2) is encodable by the transcript variant 4 (NM_001278404.2), which lacks a portion of the 5′ coding region, and uses a downstream in-frame start codon, compared to variant 1. The encoded isoform (3) has a shorter N-terminus, compared to isoform 1. Human LILRB2 isoform 4 (NP_001265334.2) is encodable by the transcript variant 5 (NM_001278405.2), which has a shorter 5′ UTR, and lacks an internal exon which results in a frameshift and an early stop codon, compared to variant 1. The encoded isoform (4) has a shorter and distinct C-terminus, compared to isoform 1. Human LILRB2 isoform 5 (NP_001265335.2) is encodable by the transcript variant 6 (NM_001278406.2), which has a shorter 5′ UTR, lacks several exons, and its 3′-terminal exon extends past a splice site that is used in variant 1. The resulting protein (isoform 5) has a shorter and distinct C-terminus, compared to isoform 1. Representative sequences of LILRB2 orthologs are presented below in Table 1.

Anti-LILRB2 antibodies suitable for detecting LILRB2 protein are well-known in the art and include, for example, antibodies sc-515288, and sc-390287 (Santa Cruz Biotechnology), antibodies MAB2078, AF2078, H00010288-M01, and NBP1-98554 (Novus Biologicals, Littleton, Colo.), antibodies ab128349, ab95819, and ab95820 (AbCam, Cambridge, Mass.), antibodies Cat #: TA349368 and TA323297 (Origene, Rockville, Md.), etc. In addition, reagents are well-known for detecting LILRB2 expression. Multiple clinical tests of LILRB2 are available in NIH Genetic Testing Registry (GTR®) (e.g., GTR Test ID: GTR000541153.2, offered by Fulgent Clinical Diagnostics Lab (Temple City, Calif.)). Moreover, multiple siRNA, shRNA, CRISPR constructs for reducing LILRB2 expression can be found in the commercial product lists of the above-referenced companies, such as siRNA product #SR323061, shRNA products #TL311729, TR311729, TG311729, TF311729, TL311729V and CRISPR products #KN207770 from Origene Technologies (Rockville, Md.), CRISPR gRNA products from Applied Biological Materials (K1215408) and from Santa Cruz (sc-401944), and RNAi products from Santa Cruz (Cat #sc-45200). It is to be noted that the term can further be used to refer to any combination of features described herein regarding LILRB2 molecules. For example, any combination of sequence composition, percentage identify, sequence length, domain structure, functional activity, etc. can be used to describe a LILRB2 molecule encompassed by the present invention.

The term “CCR5” refers to C—C Motif Chemokine Receptor 5, a member of the beta chemokine receptor family, which is predicted to be a seven transmembrane protein similar to G protein-coupled receptors. CCR5 is expressed by T cells and macrophages, and is known to be an important co-receptor for macrophage-tropic virus, including HIV, to enter host cells. Defective alleles of CCR5 gene have been associated with the HIV infection resistance. The ligands of CCR5 receptor include monocyte chemoattractant protein 2 (MCP-2), macrophage inflammatory protein 1 alpha (MIP-1 alpha), macrophage inflammatory protein 1 beta (MIP-1 beta) and regulated on activation normal T expressed and secreted protein (RANTES). Expression of CCR5 gene was also detected in a promyeloblastic cell line, indicating that this protein may play a role in granulocyte lineage proliferation and differentiation. The CCR5 gene is located at the chemokine receptor gene cluster region. Diseases associated with CCR5 include west nile virus and diabetes mellitus, insulin-dependent. Among its related pathways are cytokine signaling in immune system and akt signaling. In some embodiments, the CCR5 gene, located on chromosome 3p in humans, consists of 3 exons. Knockout mouse lines, including Ccr5^{tm1Kuz} (Huffnagle et al. (1999) J Immunol. 163:4642-4646), Ccr5^{tm1Blck} (Luckow et al. (2004) Eur J Immunol 34:2568-2578), and Ccr5^{tm1(CCR5)PfiHuman} (Amsellem et al. (2014) Circulation 130:880-891), exist. In some embodiments, CCR5 protein has 352 amino acids and/or a molecular mass of 40524 Da. The known binding partners of CCR5 include, e.g., PRAF2, CCL4, GRK2, ARRB1, ARRB2 and CNIH4.

The term “CCR5” is intended to include fragments, variants (e.g., allelic variants), and derivatives thereof. Representative human CCR5 cDNA and human CCR5 protein sequences are well-known in the art and are publicly available from the National Center for Biotechnology Information (NCBI) (see, for example, ncbi.nlm.nih.gov/gene/1234). For example, human CCR5 (NP_000570.1 and NP_001093638.1) is encodable by the transcript variant A (NM_000579.3), which is the longer transcript, and by the transcript variant B (NM_001100168.1), which differs in the 5′ UTR compared to variant A. Both variants encode the same protein. Nucleic acid and polypeptide sequences of CCR5 orthologs in organisms other than humans are well-known and include, for example, chimpanzee CCR5 (NM_001009046.1 and NP_001009046.1), rhesus monkey CCR5 (NM_001042773.3 and NP_001036238.2; and NM_001309402.1 and NP_001296331.1), dog CCR5 (NM_001012342.3 and NP_001012342.2), cattle CCR5 (NM_001011672.2 and NP_001011672.2), mouse CCR5 (NM_009917.5 and NP_034047.2), and rat CCR5 (NM_053960.3 and NP_446412.2). Representative sequences of CCR5 orthologs are presented below in Table 1.

Anti-CCR5 antibodies suitable for detecting CCR5 protein are well-known in the art and include, for example, antibodies GTX101330, GTX109635, and GTX21673 (GeneTex, Irvine, Calif.), antibodies sc-57072 and sc-55484 (Santa Cruz Biotechnology), antibodies MAB182, NBP2-31374, NBP1-41434, and MAB181 (Novus Biologicals, Littleton, Colo.), antibodies ab65850, ab1673, and ab7346 (AbCam, Cambridge, Mass.), antibodies Cat #: TA351039 and TA348418 (Origene, Rockville, Md.), etc. Other anti-CCR5 antibodies are also known and include, for example, those described in U.S. Pat. Pubis. US20010000241, US20020099176A1, US20090110686A1, and US20080107595. In addition, reagents are well-known for detecting CCR5 expression. Multiple clinical tests of CCR5 are available in NIH Genetic Testing Registry (GTR®) (e.g., GTR Test ID: GTR000516140.2, offered by Fulgent Clinical Diagnostics Lab (Temple City, Calif.)). Moreover, multiple siRNA, shRNA, CRISPR constructs for reducing CCR5 expression can be found in the commercial product lists of the above-referenced companies, such as siRNA product #SR300873, shRNA products #TL314126, TR314126, TG314126, TF 314126, TL314126V and CRISPR products #KN216008 from Origene Technologies (Rockville, Md.), CRISPR gRNA products from Applied Biological Materials (K6988308) and from Santa Cruz (sc-402548), and RNAi products from Santa Cruz (Cat #sc-35062 and sc-35063). It is to be noted that the term can further be used to refer to any combination of features described herein regarding CCR5 molecules. For example, any combination of sequence composition, percentage identify, sequence length, domain structure, functional activity, etc. can be used to describe a CCR5 molecule encompassed by the present invention.

The term “EVI2B” refers to Ecotropic Viral Integration Site 2B. EVI2B is required for granulocyte differentiation and functionality of hematopoietic progenitor cells through the control of cell cycle progression and survival of hematopoietic progenitor cells. In some embodiment, the gene EVI2B, located on chromosome 17q, consists of 3 exons. In some embodiments, human EVI2B protein has 448 amino acids and/or a molecular mass of 48666 Da.

The term “EVI2B” is intended to include fragments, variants (e.g., allelic variants), and derivatives thereof. Representative human EVI2B cDNA and human EVI2B protein sequences are well-known in the art and are publicly available from the National Center for Biotechnology Information (NCBI) (see, for example, ncbi.nlm.nih.gov/gene/2124). For example, human EVI2B (NP_006486.3) is encoded by the transcript (NM_006495.3). Nucleic acid and polypeptide sequences of EVI2B orthologs in organisms other than humans are well-known and include, for example, chimpanzee EVI2B (XM_024350668.1 and XP_024206436.1; and XM_001174747.4 and XP_001174747.1), rhesus monkey EVI2B (XM_001111968.3 and XP_00111968.1; and XM_001111891.3 and XP_001111891.1), dog EVI2B (XM_022423331.1 and XP_022279039.1; XM_022423330.1 and XP_022279038.1; XM_005624837.3 and XP_005624894.1; and XM_005624836.3 and XP_005624893.1), cattle EVI2B (NM_001099166.2 and NP_001092636.1), mouse EV12B (NM_001077496.1 and NP_001070964.1), and rat EVI2B (NM_001271482.1 and NP_001258411.1). Representative sequences of EVI2B orthologs are presented below in Table 1.

Anti-EVI2B antibodies suitable for detecting EVI2B protein are well-known in the art and include, for example, antibodies GTX79980, GTX79981, and GTX46414 (GeneTex, Irvine, Calif.), antibodies NBP1-85342, NBP2-62207, NBP1-59952, and H00002124-M02 (Novus Biologicals, Littleton, Colo.), antibodies ab101146, ab101040, and ab173149 (AbCam, Cambridge, Mass.), antibodies Cat #: TA341843 and AM12138RP-N (Origene, Rockville, Md.), etc. In addition, reagents are well-known for detecting EVI2B expression. Multiple clinical tests of EVI2B are available in NIH Genetic Testing Registry (GTR®) (e.g., GTR Test ID: GTR000535142.2, offered by Fulgent Clinical Diagnostics Lab (Temple City, Calif.)). Moreover, multiple siRNA, shRNA, CRISPR constructs for reducing EVI2B expression can be found in the commercial product lists of the above-referenced companies, such as siRNA product #SR320090, shRNA products #TL313146, TR313146, TG313146, TF313146, TL313146V and CRISPR products #KN203253 from Origene Technologies (Rockville, Md.), CRISPR gRNA products from Applied Biological Materials (K4066808) and from Santa Cruz (sc-416696), and RNAi products from Santa Cruz (Cat #sc-93673 and sc-144963). It is to be noted that the term can further be used to refer to any combination of features described herein regarding EVI2B molecules. For example, any combination of sequence composition, percentage identify, sequence length, domain structure, functional activity, etc. can be used to describe an EVI2B molecule encompassed by the present invention.

The term “CLEC7A” refers to C-Type Lectin Domain Containing 7A, which is a member of the C-type lectin/C-type lectin-like domain (CTL/CTLD) superfamily. The encoded glycoprotein is a small type II membrane receptor with an extracellular C-type lectin-like domain fold and a cytoplasmic domain with an immunoreceptor tyrosine-based activation motif. It functions as a pattern-recognition receptor that recognizes a variety of beta-1,3-linked and beta-1,6-linked glucans from fungi and plants, and in this way plays a role in innate immune response. This gene is closely linked to other CTL/CTLD superfamily members on chromosome 12p13 in humans in the natural killer gene complex region. Diseases associated with CLEC7A include aspergillosis and candidiasis, familial. Among its related pathways are CLEC7A (Dectin-1) signaling and innate immune system. In some embodiments, the gene CLEC7A, located on chromosome 12p, consists of 8 exons. Knockout mouse lines, including Clec7a^{tmlGdb} (Taylor et al. (2007) Nat Immunol 8:31-38), and Clec7a^{tm1Yiw} (Saijo et al. (2007) Nat Immumol. 8:39-46), exist. In some embodiments, human CLEC7A protein has 247 amino acids and/or a molecular mass of 27627 Da. CLEC7A protein interacts with SYK, and isoform 5 of CLEC7A interaects with RANBP9.

The term “CLEC7A” is intended to include fragments, variants (e.g., allelic variants), and derivatives thereof. Representative human CLEC7A cDNA and human CLEC7A protein sequences are well-known in the art and are publicly available from the National Center for Biotechnology Information (NCBI) (see, for example, ncbi.nlm.nih.gov/gene/64581). For example, at least six different human CLEC7A isoforms are known. Human CLEC7A isoform a (NP_922938.1) is encodable by the transcript variant 1 (NM_197947.2), which is the longest transcript. Human CLEC7A isoform b (NP_072092.2) is encodable by the transcript variant 2 (NM_022570.4), lacks an alternate in-frame exon compared to variant 1, resulting in a shorter protein (isoform b) compared to isoform a. Human CLEC7A isoform c (NP_922939.1) is encodable by the transcript variant 3 (NM_197948.2), which lacks an alternate exon, which results in a frameshift and an early stop codon, compared to variant 1. The resulting protein (isoform c) is shorter and has a distinct C-terminus, compared to isoform a. Human CLEC7A isoform d (NP_922940.1) is encodable by the transcript variant 4 (NM_197949.2), which lacks two alternate exons, which results in a frameshift and an early stop codon, compared to variant 1. The resulting protein (isoform d) is shorter and contains a distinct C-terminus, compared to isoform a. Human CLEC7A isoform e (NP_922941.1) is encodable by the transcript variant 5 (NM_197950.2), which lacks an alternate in-frame exon compared to variant 1, resulting in a shorter protein (isoform e) compared to isoform a. Human CLEC7A isoform f (NP_922945.1) is encodable by the transcript variant 6 (NM_197954.2), has multiple differences in the coding region, compared to variant 1, one of which results in an early stop codon. The resulting protein (isoform f) has a distinct C-terminus and is much shorter than isoform a. Nucleic acid and polypeptide sequences of CLEC7A orthologs in organisms other than humans are well-known and include, for example, chimpanzee CLEC7A (XM_016922965.2 and XP_016778454.1; XM_001144689.3 and XP_001144689.1; XM_001144825.3 and XP_001144825.1; XM_003313487.4 and XP_003313535.1; XM_528732.4 and XP_528732.2; and XM_001144313.4 and XP_001144313.1), rhesus monkey CLEC7A (NM_001032943.1 and NP_001028115.1), dog CLEC7A (XM_022411028.1 and XP_022266736.1; XM_849050.3 and XP_854143.1; and XM_005637163.2 and XP_005637220.1), cattle CLEC7A (NM_001031852.1 and NP_001027022.1), mouse CLEC7A (NM_001309637.1 and NP_001296566.1; and NM_020008.3 and NP_064392.2), and rat CLEC7A (NM_001173386.1 and NP_001166857.1). Representative sequences of CLEC7A orthologs are presented below in Table 1.

Anti-CLEC7A antibodies suitable for detecting CLEC7A protein are well-known in the art and include, for example, antibodies GTX41467, GTX41471, and GTX41466 (GeneTex, Irvine, Calif.), antibodies MAB1859, AF1859, NBP1-45514, and NBP2-41170 (Novus Biologicals, Littleton, Colo.), antibodies ab140039, ab82888, and ab189968 (AbCam, Cambridge, Mass.), antibodies Cat #: TA322197 and TA320003 (Origene, Rockville, Md.), etc. Other anti-CLEC7A antibodies are also known and include, for example, those described in U.S. Pat. Pubis. US20140322214A1 and US20170095573A1, and U.S. Pat. Nos. 7,915,041B2. In addition, reagents are well-known for detecting CLEC7A expression. Multiple clinical tests of CLEC7A are available in NIH Genetic Testing Registry (GTR®) (e.g., GTR Test ID: GTR000516241.2, offered by Fulgent Clinical Diagnostics Lab (Temple City, Calif.)). Moreover, multiple siRNA, shRNA, CRISPR constructs for reducing CLEC7A expression can be found in the commercial product lists of the above-referenced companies, such as siRNA product #SR312068, shRNA products #TL305354, TR305354, TG305354, TF305354, TL305354V and CRISPR products #KN214107 from Origene Technologies (Rockville, Md.), CRISPR gRNA products from Applied Biological Materials (K6685408) and from Santa Cruz (sc-417053), and RNAi products from Santa Cruz (Cat #sc-63276 and sc-63277). It is to be noted that the term can further be used to refer to any combination of features described herein regarding CLEC7A molecules. For example, any combination of sequence composition, percentage identify, sequence length, domain structure, functional activity, etc. can be used to describe a CLEC7A molecule encompassed by the present invention.

The term “TBXAS” refers to Thromboxane A Synthase 1, which is a member of the cytochrome P450 superfamily of enzymes. The cytochrome P450 proteins are monooxygenases which catalyze many reactions involved in drug metabolism and synthesis of cholesterol, steroids and other lipids. However, this protein is considered a member of the cytochrome P450 superfamily on the basis of sequence similarity rather than functional similarity. This endoplasmic reticulum membrane protein catalyzes the conversion of prostglandin H2 to thromboxane A2, a potent vasoconstrictor and inducer of platelet aggregation. The enzyme plays a role in several pathophysiological processes including hemostasis, cardiovascular disease, and stroke. Diseases associated with TBXAS1 include ghosal hematodiaphyseal dysplasia and bleeding disorder, platelet-type, 14. Among its related pathways are platelet activation and metabolism. In some embodiments, the gene TBXAS1, located on chromosome 7q, consists of 23 exons. Knockout mouse lines, including Tbxas1^{tmlSwl} (Yu et al. (2004) Blood 104:135-142), and Tbxas1^{tmlOkunHuman} (Matsunobu et al. (2013) J Lipid Res 54:2979-2987), exist. In some embodiments, TBXAS1 protein has 533 amino acids and/or a molecular mass of 60518 Da.

The term “TBXAS1” is intended to include fragments, variants (e.g., allelic variants), and derivatives thereof. Representative human TBXAS1 cDNA and human TBXAS1 protein sequences are well-known in the art and are publicly available from the National Center for Biotechnology Information (NCBI) (see, for example, ncbi.nlm.nih.gov/gene/6916). For example, at least five different human TBXAS1 isoforms are known. Human TBXAS1 isoform 1 (NP_001052.2 and NP_001124438.1) is encodable by the transcript variant 1 (NM_001061.4) and transcript variant 3 (NM_001130966.2). This variant (3, also known as TXS-III) differs in the 5′ UTR, compared to variant 1. Both variants 1 and 3 encode the same isoform (1, also known as isoform TXS-I). Human TBXAS1 isoform 2 (NP_112246.2) is encodable by the transcript variant 2 (NM_030984.3), which lacks an alternate exon in the 3′ coding region that encodes the heme binding site, compared to transcript variant 1. The encoded isoform (2, also known as isoform TXS-II) lacks thromboxane A synthase activity, has a distinct C-terminus, and is shorter than isoform 1. Human TBXAS1 isoform 3 (NP_001159725.1) is encodable by the transcript variant 4 (NM_001166253.1), which includes an alternate in-frame exon in the central coding region, compared to variant 1, resulting in an isoform (3) that is longer than isoform 1. Human TBXAS isoform 4 (NP_001159726.1) is encodable by the transcript variant 5 (NM_001166254.1), which differs in the 5′ UTR, lacks a portion of the 5′ coding region, and uses a downstream translational start codon, compared to variant 1. The encoded isoform (4) is shorter at the N-terminus, compared to isoform 1. Human TBXAS1 isoform 5 (NP_001300957.1) is encodable by the transcript variant 6 (NM_001314028.1), which uses an alternate splice site in an internal exon, compared to variant 1. The resulting isoform (5) has a shorter and distinct N-terminus compared to isoform 1. Nucleic acid and polypeptide sequences of TBXAS1 orthologs in organisms other than humans are well-known and include, for example, dog TBXAS1 (XM_005629559.2 and XP_005629616.1; XM_539887.5 and XP_539887.2; XM_014119949.2 and XP_013975424.1; and XM_022403739.1 and XP_022259447.1), cattle TBXAS1 (NM_001046027.2 and NP_001039492.1), mouse TBXAS1 (NM_011539.3 and NP_035669.3), rat TBXAS1 (NM_012687.1 and NP_036819.1), chicken TBXAS1 (XM_416334.6 and XP_416334.4; XM_004937846.3 and XP_004937903.2; and XM_025155784.1 and XP_025011552.1), tropical clawed frog TBXAS1 (NM_001171526.1 and NP_001164997.1), and zebrafish TBXAS1 (NM_205609.2 and NP_991172.2). Representative sequences of TBXAS1 orthologs are presented below in Table 1.

Anti-TBXAS1 antibodies suitable for detecting TBXAS1 protein are well-known in the art and include, for example, antibodies GTX83523, GTX83521, and GTX83522 (GeneTex, Irvine, Calif.), antibodies NBP2-02710, NBP2-33948, NBP2-33946, and NBP2-33947 (Novus Biologicals, Littleton, Colo.), antibodies ab39362, ab187176, and ab157481 (AbCam, Cambridge, Mass.), antibodies Cat #: CF501380 and AP51174PU-N(Origene, Rockville, Md.), etc. In addition, reagents are well-known for detecting TBXAS1 expression. Multiple clinical tests of TBXAS1 are available in NIH Genetic Testing Registry (GTR®) (e.g., GTR Test ID: GTR000518496.2, offered by Fulgent Clinical Diagnostics Lab (Temple City, Calif.)). Moreover, multiple siRNA, shRNA, CRISPR constructs for reducing TBXAS1 expression can be found in the commercial product lists of the above-referenced companies, such as siRNA product #SR304732, shRNA products #TL301186, TR301186, TG301186, TF301186, TL301186V and CRISPR products #KN208028 from Origene Technologies (Rockville, Md.), CRISPR gRNA products from Applied Biological Materials (K6806708) and from Santa Cruz (sc-418609), and RNAi products from Santa Cruz (Cat #sc-62451 and sc-76779). It is to be noted that the term can further be used to refer to any combination of features described herein regarding TBXAS1 molecules. For example, any combination of sequence composition, percentage identify, sequence length, domain structure, functional activity, etc. can be used to describe a TBXAS1 molecule encompassed by the present invention.

The term “SIGLEC7” refers to Sialic Acid Binding Ig Like Lectin 7, which is a putative adhesion molecule that mediates sialic-acid dependent binding to cells. SIGLEC7 preferentially binds to alpha-2,3- and alpha-2,6-linked sialic acid. SIGLEC7 also binds disialogangliosides (disialogalactosyl globoside, disialyl lactotetraosylceramide and disialyl GalNAc lactotetraoslylceramide). The sialic acid recognition site of SIGLEC7 may be masked by cis interactions with sialic acids on the same cell surface. In the immune response, SIGLEC7 may act as an inhibitory receptor upon ligand induced tyrosine phosphorylation by recruiting cytoplasmic phosphatase(s) via their SH2 domain(s) that block signal transduction through dephosphorylation of signaling molecules. SIGLEC7 mediates inhibition of natural killer cells cytotoxicity. SIGLEC7 may play a role in hemopoiesis. SIGLEC7 inhibits differentiation of CD34+ cell precursors towards myelomonocytic cell lineage and proliferation of leukemic myeloid cells in vitro. Diseases associated with SIGLEC7 include pheochromocytoma. Among its related pathways are hematopoietic stem cell differentiation pathways and lineage-specific markers and innate immune system. In some embodiments, the gene SIGLEC7, located on chromosome 19q, consists of 7 exons. In some embodiments, human SIGLEC7 protein has 467 amino acids and/or a molecular mass of 51143 Da. In some embodiments, SIGLEC7 protein contains 1 copy of a cytoplasmic motif that is referred to as the immunoreceptor tyrosine-based inhibitor motif (ITIM). This motif is involved in modulation of cellular responses. The phosphorylated ITIM motif can bind the SH2 domain of several SH2-containing phosphatases. SIGLEC7 protein interacts with PTPN6/SHP-1 upon phosphorylation.

The term “SIGLEC7” is intended to include fragments, variants (e.g., allelic variants), and derivatives thereof. Representative human SIGLEC7 cDNA and human SIGLEC7 protein sequences are well-known in the art and are publicly available from the National Center for Biotechnology Information (NCBI) (see, for example, ncbi.nlm.nih.gov/gene/27036). For example, at least three different human SIGLEC7 isoforms are known. Human SIGLEC7 isoform 1 (NP_055200.1), the longest isoform, is encodable by the transcript variant 1 (NM_014385.3). Human SIGLEC7 isoform 2 (NP_057627.2) is encodable by the transcript variant 2 (NM_016543.3), which lacks an in-frame coding exon, compared to variant 1. The resulting isoform (2) lacks an internal segment, compared to isoform 1. Human SIGLEC7 isoform 3 (NP_001264130.1) is encodable by the transcript variant 3 (NM_001277201.1), which lacks all internal coding exons, compared to variant 1. The resulting isoform (3) is C-terminal truncated, compared to isoform 1. Nucleic acid and polypeptide sequences of SIGLEC7 orthologs in organisms other than humans are well-known and include, for example, chimpanzee SIGLEC7 (XM_016936700.1 and XP_016792189.1; and XM_016936701.1 and XP_016792190.1). Representative sequences of SIGLEC7 orthologs are presented below in Table 1.

Anti-SIGLEC7 antibodies suitable for detecting SIGLEC7 protein are well-known in the art and include, for example, antibodies GTX107080, GTX116337, and GTX53005 (GeneTex, Irvine, Calif.), antibodies sc-398919 and sc-398181 (Santa Cruz Biotechnology), antibodies AF1138, MAB1138, MAB11381, and NBP2-20360 (Novus Biologicals, Littleton, Colo.), antibodies ab38573, ab38574, and ab111619 (AbCam, Cambridge, Mass.), antibodies Cat #: AM05592FC-N and AM05592PU-L (Origene, Rockville, Md.), etc. Other anti-SIGLEC7 antibodies are also known and include, for example, those described in U.S. Pat. Pubs. US20170306014, US20190085077, US20190023786, and US20180244770. In addition, reagents are well-known for detecting SIGLEC7 expression. Multiple clinical tests of SIGLEC7 are available in NIH Genetic Testing Registry (GTR®) (e.g., GTR Test ID: GTR000546879.2, offered by Fulgent Clinical Diagnostics Lab (Temple City, Calif.)). Moreover, multiple siRNA, shRNA, CRISPR constructs for reducing SIGLEC7 expression can be found in the commercial product lists of the above-referenced companies, such as siRNA product #SR308944, shRNA products #TL309445, TR309445, TG309445, TF309445, TL309445V and CRISPR products #KN206995 from Origene Technologies (Rockville, Md.), CRISPR gRNA products from Applied Biological Materials (K2147608) and from Santa Cruz (sc-407464), and RNAi products from Santa Cruz (Cat #sc-106757 and sc-106757-SH). It is to be noted that the term can further be used to refer to any combination of features described herein regarding SIGLEC7 molecules. For example, any combination of sequence composition, percentage identify, sequence length, domain structure, functional activity, etc. can be used to describe a SIGLEC7 molecule encompassed by the present invention.

The term “DOCK2” refers to Dedicator Of Cytokinesis 2. DOCK2 belongs to the CDM protein family. It is specifically expressed in hematopoietic cells and is predominantly expressed in peripheral blood leukocytes. The protein is involved in remodeling of the actin cytoskeleton required for lymphocyte migration in response to chemokine signaling. It activates members of the Rho family of GTPases, for example RAC1 and RAC2, by acting as a guanine nucleotide exchange factor (GEF) to exchange bound GDP for free GTP. DOCK2 is involved in cytoskeletal rearrangements required for lymphocyte migration in response of chemokines. DOCK2 activates RAC1 and RAC2, but not CDCl42, by functioning as a guanine nucleotide exchange factor (GEF), which exchanges bound GDP for free GTP. DOCK2 also participates in IL2 transcriptional activation via the activation of RAC2. Knockout mouse lines, called Dock2^{TmlTsas} (Fukui et al. (2001) Nature 412:826-831), and Dock2^{tmlYsfk} (Kunisaki et al. (2006) J Cell Biol 174:647-652), exist. In some embodiments, the gene DOCK2, located on chromosome 5q in humans, consists of 59 exons. The DOCK2 gene is conserved in chimpanzee, dog, cow, mouse, rat, chicken, and frog. In some embodiments, human DOCK2 protein has 1830 amino acids and/or a molecular mass of 211948 Da. The known binding partners of DOCK2 include, e.g., RAC1, RAC2, CRKL, VAV, and CD3Z.

The term “DOCK2” is intended to include fragments, variants (e.g., allelic variants), and derivatives thereof. Representative human DOCK2 cDNA and human DOCK2 protein sequences are well-known in the art and are publicly available from the National Center for Biotechnology Information (NCBI) (see, for example, ncbi.nlm.nih.gov/gene/80231). For example, human DOCK2 (NP_004937.1) is encodable by the transcript (NM_004946.3). Nucleic acid and polypeptide sequences of DOCK2 orthologs in organisms other than humans are well-known and include, for example, chimpanzee DOCK2 (XM_016954161.2 and XP_016809650.1; XM_016954163.2 and XP_016809652.1; XM_016954162.2 and XP_016809651.1; and XM_016954164.2 and XP_016809653.1), dog DOCK2 (XM_546246.5 and XP_546246.3), cattle DOCK2 (XM_024981420.1 and XP_024837188.1 and XM_024981421.1 and XP_024837189.1), mouse DOCK2 (NM_033374.3 and NP_203538.2), rat DOCK2 (XM_008767630.2 and XP_008765852.1), chicken DOCK2 (XM_425184.6 and XP_425184.4), and tropical clawed frog DOCK2 (XM_018092631.1 and XP_017948120.1). Representative sequences of DOCK2 orthologs are presented below in Table 1.

Anti-DOCK2 antibodies suitable for detecting DOCK2 protein are well-known in the art and include, for example, antibodies TA340057 and TA802698 (OriGene, Rockville, Md.), antibodies NBP2-46468 and NBP2-38303 (Novus Biologicals, Littleton, Colo.), antibodies ab74659, ab226797, and ab203068 (AbCam, Cambridge, Mass.), etc. In addition, reagents are well-known for detecting DOCK2 expression. Multiple clinical tests of DOCK2 are available in NIH Genetic Testing Registry (GTR®) (e.g., GTR Test ID: GTR000536814.1, offered by Fulgent Clinical Diagnostics Lab (Temple City, Calif.)). Moreover, multiple siRNA, shRNA, CRISPR constructs for reducing DOCK2 expression can be found in the commercial product lists of the above-referenced companies, such as siRNA product #SR301250, shRNA products #TL313396, TR313396, TG313396, TF313396, TL313396V and CRISPR products #KN211198 from Origene Technologies (Rockville, Md.), CRISPR gRNA products from Applied Biological Materials (K3865908) and from Santa Cruz (sc-407692), and RNAi products from Santa Cruz (Cat #sc-60545 and sc-60546). It is to be noted that the term can further be used to refer to any combination of features described herein regarding DOCK2 molecules. For example, any combination of sequence composition, percentage identify, sequence length, domain structure, functional activity, etc. can be used to describe a DOCK2 molecule encompassed by the present invention.

The term “CD53” refers to CD53 molecule, which is a member of the transmembrane 4 superfamily, also known as the tetraspanin family. Most of these members are cell-surface proteins that are characterized by the presence of four hydrophobic domains. The proteins mediate signal transduction events that play a role in the regulation of cell development, activation, growth and motility. This encoded protein is a cell surface glycoprotein that is known to complex with integrins. It contributes to the transduction of CD2-generated signals in T cells and natural killer cells and has been suggested to play a role in growth regulation. Familial deficiency of this gene has been linked to an immunodeficiency associated with recurrent infectious diseases caused by bacteria, fungi and viruses. Diseases associated with CD53 include intestinal tuberculosis and gastrointestinal tuberculosis. Among its related pathways are innate immune system. CD53 is required for efficient formation of myofibers in regenerating muscle at the level of cell fusion. CD53 may be involved in growth regulation in hematopoietic cells. In some embodiments, the gene CD53, located on chromosome 1p, consists of 9 exons. In some embodiments, human CD53 protein has 219 amino acids and/or a molecular mass of 24341 Da.

The term “CD53” is intended to include fragments, variants (e.g., allelic variants), and derivatives thereof. Representative human CD53 cDNA and human CD53 protein sequences are well-known in the art and are publicly available from the National Center for Biotechnology Information (NCBI) (see, for example, ncbi.nlm.nih.gov/gene/963). For example, at least two different human CD53 isoforms are known. Human CD53 isoform 1 (NP_000551.1 and NP_001035122.1) is encodable by the transcript variant 1 (NM_001040033.1), which represents the longer transcript, and by the transcript variant 2 (NM_000560.3), which differs in the 5′ UTR, compared to variant 1. Variants 1 and 2 encode the same protein. Human CD53 isoform 2 (NP_001307567.1) is encodable by the transcript variant 3 (NM_001320638.1), which differs in the 5′ UTR and lacks exons in the coding region, compared to variant 1. The encoded isoform (2) is shorter, compared to isoform 1. Nucleic acid and polypeptide sequences of CD53 orthologs in organisms other than humans are well-known and include, for example, chimpanzee CD53 (XM_003308334.3 and XP_003308382.1; XM_016925800.1 and XP_016781289.1; and XM_009429624.2 and XP_009427899.1), rhesus monkey CD53 (XM_015148031.1 and XP_015003517.1, XM_001102190.3 and XP_001102190.1, and XM_015148036.1 and XP_015003522.1), dog CD53 (XM_003639132.3 and XP_003639180.1), cattle CD53 (NM_001034232.2 and NP_001029404.1), mouse CD53 (NM_007651.3 and NP_031677.1), and rat CD53 (NM_012523.2 and NP_036655.1). Representative sequences of CD53 orthologs are presented below in Table 2.

Anti-CD53 antibodies suitable for detecting CD53 protein are well-known in the art and include, for example, antibodies GTX34220, GTX79940, and GTX79942 (GeneTex, Irvine, Calif.), antibodies sc-390185 and sc-73365 (Santa Cruz Biotechnology), antibodies MAB4624, NB500-393, NBP2-44609, and NBP2-14464 (Novus Biologicals, Littleton, Colo.), antibodies ab134094, ab68565, and ab213083 (AbCam, Cambridge, Mass.), antibodies Cat #: SM1137AS and SM1137LE (Origene, Rockville, Md.), etc. In addition, reagents are well-known for detecting CD53 expression. Multiple clinical tests of CD53 are available in NIH Genetic Testing Registry (GTR®) (e.g., GTR Test ID: GTR000532965.2, offered by Fulgent Clinical Diagnostics Lab (Temple City, Calif.)). Moreover, multiple siRNA, shRNA, CRISPR constructs for reducing CD53 expression can be found in the commercial product lists of the above-referenced companies, such as siRNA product #SR300686, shRNA products #TL314077, TR314077, TG314077, TF314077, TL314077V and CRISPR products #KN208095 from Origene Technologies (Rockville, Md.), CRISPR gRNA products from Applied Biological Materials (K6868708) and from Santa Cruz (sc-405861), and RNAi products from Santa Cruz (Cat #sc-42796 and sc-42797). It is to be noted that the term can further be used to refer to any combination of features described herein regarding CD53 molecules. For example, any combination of sequence composition, percentage identify, sequence length, domain structure, functional activity, etc. can be used to describe a CD53 molecule encompassed by the present invention.

The term “FERMT3” refers to Fermitin Family Member 3 and belongs to a small family of proteins that mediate protein-protein interactions involved in integrin activation and thereby have a role in cell adhesion, migration, differentiation, and proliferation. FERMT3 protein has a key role in the regulation of hemostasis and thrombosis. It may also help maintain the membrane skeleton of erythrocytes. Mutations in FERMT3 gene cause the autosomal recessive leukocyte adhesion deficiency syndrome-III (LAD-III). FERMT3 plays a central role in cell adhesion in hematopoietic cells (Svensson et al. (2009) Nat Med 15:306-312; Suratannon et al. (2016) Pediatr Allergy Immunol 27:214-217). FERMT3 acts by activating the integrin beta-1-3 (ITGB1, ITGB2 and ITGB3). FERMT3 is required for integrin-mediated platelet adhesion and leukocyte adhesion to endothelial cells (Malinin et al. (2009) Nat Med 15:313-318), and for activation of integrin beta-2 (ITGB2) in polymorphonuclear granulocytes (PMNs). Human isoform 2 of FERMT3 may act as a repressor of NF-kappa-B and apoptosis. In some embodiments, the gene FERMT3, located on chromosome 11q, consists of 16 exons. Knockout mouse lines, including Fermt3^{tmlRef} (Moser et al. (2008) Nat Med. 14:325-330), Fermt3^{tm2.Ref} (Cohen et al. (2013) Blood 122:2609-2617), and Fermt3^{tmlb(KOMP)Wtsi} (International Knockout Mouse Consortium), exist. In some embodiments, human FERMT3 protein has 667 amino acids and/or a molecular mass of 75953 Da. FERMT3 interacts with ITGB1, ITGB2 and ITGB3 via cytoplasmic tails.

The term “FERMT3” is intended to include fragments, variants (e.g., allelic variants), and derivatives thereof. Representative human FERMT3 cDNA and human FERMT3 protein sequences are well-known in the art and are publicly available from the National Center for Biotechnology Information (NCBI) (see, for example, ncbi.nlm.nih.gov/gene/83706). For example, at least two different human FERMT3 isoforms are known. Human FERMT3 isoform 1 (NP_848537.1) is encodable by the transcript variant 1 (NM_178443.2), which represents the longer transcript. Human FERMT3 isoform 2 (NP_113659.3) is encodable by the transcript variant 2 (NM_031471.5), which uses an alternate in-frame splice junction at the 5′ end of a coding exon compared to variant 1. The resulting isoform (2) has the same N- and C-termini but is shorter compared to the long isoform (1). Nucleic acid and polypeptide sequences of FERMT3 orthologs in organisms other than humans are well-known and include, for example, chimpanzee FERMT3 (XM_009423350.3 and XP_009421625.1; and XM_508522.6 and XP_508522.3), rhesus monkey FERMT3 (XM_015113900.1 and XP_014969386.1, and XM_015113898.1 and XP_014969384.1), dog FERMT3 (XM_003639655.3 and XP_003639703.1), mouse FERMT3 (NM_001362399.1 and NP_001349328.1, and NM_153795.2 and NP_722490.1), rat FERMT3 (NM_001127543.1 and NP_001121015.1); and zebrafsh FERMT3 (NM_200904.2 and NP_957198.2). Representative sequences of FERMT3 orthologs are presented below in Table 2.

Anti-FERMT3 antibodies suitable for detecting FERMT3 protein are well-known in the art and include, for example, antibodies GTX116828, GTX85027, and GTX88332 (GeneTex, Irvine, Calif.), antibodies NBP2-45641, AF7004, NBP2-20821, and H00083706-B01P (Novus Biologicals, Littleton, Colo.), antibodies ab68040, ab126900, and ab173416 (AbCam, Cambridge, Mass.), antibodies Cat #: CF807994 and TA807994 (Origene, Rockville, Md.), etc. In addition, reagents are well-known for detecting FERMT3 expression. Multiple clinical tests of FERMT3 are available in NIH Genetic Testing Registry (GTR®) (e.g., GTR Test ID: GTR000516681.2, offered by Fulgent Clinical Diagnostics Lab (Temple City, Calif.)). Moreover, multiple siRNA, shRNA, CRISPR constructs for reducing FERMT3 expression can be found in the commercial product lists of the above-referenced companies, such as siRNA product #SR313216, shRNA products #TL307798, TR307798, TG307798, TF307798, TL307798V and CRISPR products #KN202580 from Origene Technologies (Rockville, Md.), CRISPR gRNA products from Applied Biological Materials (K7584608) and from Santa Cruz (sc-408381), and RNAi products from Santa Cruz (Cat #sc-96761 and sc-146483). It is to be noted that the term can further be used to refer to any combination of features described herein regarding FERMT3 molecules. For example, any combination of sequence composition, percentage identify, sequence length, domain structure, functional activity, etc. can be used to describe a FERMT3 molecule encompassed by the present invention.

The term “CD37” refers to CD37, which is a member of the transmembrane 4 superfamily, also known as the tetraspanin family. Most of these members are cell-surface proteins that are characterized by the presence of four hydrophobic domains. The proteins mediate signal transduction events that play a role in the regulation of cell development, activation, growth and motility. CD37 protein is a cell surface glycoprotein that is known to complex with integrins and other transmembrane 4 superfamily proteins. CD37 may play a role in T-cell-B-cell interactions. A knockout mouse line, called CD37^{tm1Hor}, exists (Knobeloch et al. (2000) Mol Cell Biol 20:5363-5369). In some embodiments, the gene CD37, located on chromosome 19q, consists of 8 exons. In some embodiments, human CD37 protein has 281 amino acids and/or a molecular mass of 31703 Da. In some embodiments, CD37 interacts with SCIMP.

The term “CD37” is intended to include fragments, variants (e.g., allelic variants), and derivatives thereof. Representative human CD37 cDNA and human CD37 protein sequences are well-known in the art and are publicly available from the National Center for Biotechnology Information (NCBI) (see, for example, ncbi.nlm.nih.gov/gene/951). For example, at least two different human CD37 isoforms are known. Human CD37 isoform A (NP_001765.1) is encodable by the transcript variant 1 (NM_001774.2), which represents the longer transcript. Human CD37 isoform B (NP_001035120.1) is encodable by the transcript variant 2 (NM_001040031.1), which lacks an alternate in-frame segment in the 5′ coding region and uses a downstream start codon, compared to variant 1. The encoded isoform (B) has a shorter N-terminus, compared to isoform A. Nucleic acid and polypeptide sequences of CD37 orthologs in organisms other than humans are well-known and include, for example, chimpanzee CD37 (XM_016947061.2 and XP_016802550.1; XM_016947063.2 and XP_016802552.1; XM_016947062.2 and XP_016802551.1; and XM_016947064.2 and XP_016802553.1), rhesus monkey CD37 (XM_015124560.1 and XP_014980046.1; XM_001114865.3 and XP_001114865.2; XM_015124562.1 and XP_014980048.1; and XM_015124563.1 and XP_014980049.1), dog CD37 (XM_014118925.2 and XP_013974400.1; XM_541497.5 and XP_541497.2; and XM_005616317.3 and XP_005616374.1), cattle CD37 (NM_001046011.2 and NP_001039476.1), mouse CD37 (NM_001290802.1 and NP_001277731.1, NM_001290804.1 and NP_001277733.1, and NM_007645.4 and NP_031671.1), rat CD37 (NM_017124.1 and NP_058820.1), and tropical clawed frog CD37 (NM_001015801.2 and NP_001015801.2). Representative sequences of CD37 orthologs are presented below in Table 2.

Anti-CD37 antibodies suitable for detecting CD37 protein are well-known in the art and include, for example, antibodies GTX129598, GTX19701, and GTX83137 (GeneTex, Irvine, Calif.), antibodies sc-73364 and sc-23924 (Santa Cruz Biotechnology), antibodies NBP1-28869, NBP2-33969, NBP2-33970, and MAB4625 (Novus Biologicals, Littleton, Colo.), antibodies ab170238, ab213068, and ab227624 (AbCam, Cambridge, Mass.), antibodies Cat #: AM06314SU-N and AM32392PU-N(Origene, Rockville, Md.), etc. Other anti-CD37 antibodies are also known and include, for example, those described in U.S. Pat. Publs. US20160051694A1, US20100189722, US20180186876, and US20140348745, and U.S. Pat. Nos. 8,333,966B2 and 8,765,917B2. In addition, reagents are well-known for detecting CD37 expression. Multiple clinical tests of CD37 are available in NIH Genetic Testing Registry (GTR®) (e.g., GTR Test ID: GTR000532008.2, offered by Fulgent Clinical Diagnostics Lab (Temple City, Calif.)). Moreover, multiple siRNA, shRNA, CRISPR constructs for reducing CD37 expression can be found in the commercial product lists of the above-referenced companies, such as siRNA product #SR300873, shRNA products #TL314089, TR314089, TG314089, TF314089, TL314089V and CRISPR products #KN210768 from Origene Technologies (Rockville, Md.), CRISPR gRNA products from Applied Biological Materials (K6910708) and from Santa Cruz (sc-404423), and RNAi products from Santa Cruz (Cat #sc-42784 and sc-44663). It is to be noted that the term can further be used to refer to any combination of features described herein regarding CD37 molecules. For example, any combination of sequence composition, percentage identify, sequence length, domain structure, functional activity, etc. can be used to describe a CD37 molecule encompassed by the present invention.

The term “CXorf21” refers to Chromosome X Open Reading Frame 21. In some embodiments, the gene CXorf21, located on chromosome Xp in humans, consists of 3 exons. The CXorf21 gene is conserved in chimpanzee, rhesus monkey, dog, cow, mouse, rat, chicken, zebrafish, and frog. In some embodiments, human CXorf21 protein has 301 amino acids and/or a molecular mass of 33894 Da.

The term “CXorf21” is intended to include fragments, variants (e.g., allelic variants), and derivatives thereof. Representative human CXorf21 cDNA and human CXorf21 protein sequences are well-known in the art and are publicly available from the National Center for Biotechnology Information (NCBI) (see, for example, ncbi.nlm.nih.gov/gene/80231). For example, human CXorf21 (NP_079435.1) is encodable by the transcript (NM_025159.2). Nucleic acid and polypeptide sequences of CXorf21 orthologs in organisms other than humans are well-known and include, for example, chimpanzee CXorf21 (XM_001134922.2 and XP_001134922.1), rhesus monkey CXorf21 (NM_001194018.1 and NP_001180947.1), dog CXorf21 (XM_005641222.3 and XP_005641279.1; XM_005641223.3 and XP_005641280.1; XM_022416085.1 and XP_022271793.1; and XM_022416084.1 and XP_022271792.1), cattle CXorf21 (NM_001038537.2 and NP_001033626.1), mouse CXorf21 (NM_001163539.1 and NP_001157011.1), rat CXorf21 (NM_001109318.1 and NP_001102788.1), and chicken CXorf21 (XM_003640512.4 and XP_003640560.1). Representative sequences of CXorf21 orthologs are presented below in Table 2.

Anti-CXorf21 antibodies suitable for detecting CXorf21 protein are well-known in the art and include, for example, antibodies NBP1-82317 and H00080231-B01P (Novus Biologicals, Littleton, Colo.), antibody ab69152 (AbCam, Cambridge, Mass.), etc. In addition, reagents are well-known for detecting CXorf21 expression. Multiple clinical tests of CXorf21 are available in NIH Genetic Testing Registry (GTR®) (e.g., GTR Test ID: GTR000537724.2, offered by Fulgent Clinical Diagnostics Lab (Temple City, Calif.)). Moreover, multiple siRNA, shRNA, CRISPR constructs for reducing CXorf21 expression can be found in the commercial product lists of the above-referenced companies, such as siRNA product #SR312858, shRNA products #TL314126, TR305156, TG305156, TF305156, TL305156V and CRISPR products #KN204618 and KN300469 from Origene Technologies (Rockville, Md.), CRISPR gRNA products from Applied Biological Materials (K0537008) and from Santa Cruz (sc-413367), and RNAi products from Santa Cruz (Cat #sc-91192 and sc-140364). It is to be noted that the term can further be used to refer to any combination of features described herein regarding CXorf21 molecules. For example, any combination of sequence composition, percentage identify, sequence length, domain structure, functional activity, etc. can be used to describe a CXorf21 molecule encompassed by the present invention.

TABLE 1
SIGLEC9
VSIG4
CD74
CD207
LRRC25
SELPLG
AIF1
CD84
IGSF6
CD48
CD33
LST1
TNFAIP8L2 (TIPE2)
SPI1 (PU.1)
LILRB2
CCR5
EVI2B
CLEC7A
TBXAS1
SIGLEC7
DOCK2
SEQ ID NO: 1 Human SIGLEC9 Transcript Variant 1 cDNA Sequence
(NM_001198558.1; CDS: 96-1535)

| | |
|---|---|
| 1 | tagggcctcc tctaagtctt gagcccgcag ttcctgagag aagaaccctg aggaacagac |
| 61 | gttccctcgc ggccctggca cctctaaccc cagacatgct gctgctgctg ctgcccctgc |
| 121 | tctgggggag ggagagggcg gaaggacaga caagtaaact gctgacgatg cagagttccg |
| 181 | tgacggtgca ggaaggcctg tgtgtccatg tgccctgctc cttctcctac ccctcgcatg |
| 241 | gctggattta ccctggccca gtagttcatg gctactggtt ccgggaaggg gccaatacag |
| 301 | accaggatgc tccagtggcc acaaacaacc cagctcgggc agtgtgggag gagactcggg |
| 361 | accgattcca cctccttggg gacccacata ccaagaattg caccctgagc atcagagatg |
| 421 | ccagaagaag tgatgcgggg agatacttct ttcgtatgga gaaaggaagt ataaaatgga |
| 481 | attataaaca tcaccggctc tctgtgaatg tgacagcctt gacccacagg cccaacatcc |
| 541 | tcatcccagg caccctggag tccggctgcc cccagaatct gacctgctct gtgccctggg |
| 601 | cctgtgagca ggggacaccc cctatgatct cctggatagg gacctccgtg tcccccctgg |
| 661 | acccctccac cacccgctcc tcggtgctca ccctcatccc acagccccag gaccatggca |
| 721 | ccagcctcac ctgtcaggtg accttccctg gggccagcgt gaccacgaac aagaccgtcc |
| 781 | atctcaacgt gtcctacccg cctcagaact tgaccatgac tgtcttccaa ggagacggca |
| 841 | cagtatccac agtcttggga aatggctcat ctctgtcact cccagagggc cagtctctgc |
| 901 | gcctggtctg tgcagttgat gcagttgaca gcaatccccc tgccaggctg agcctgagct |
| 961 | ggagaggcct gaccctgtgc ccctcacagc cctcaaaccc gggggtgctg gagctgcctt |
| 1021 | gggtgcacct gagggatgca gctgaattca cctgcagagc tcagaaccct ctcggctctc |
| 1081 | agcaggtcta cctgaacgtc tccctgcaga gcaaagccac atcaggagtg actcaggggg |
| 1141 | tggtcggggg agctggagcc acagccctgg tcttcctgtc cttctgcgtc atcttcgttg |
| 1201 | tagtgaggtc ctgcaggaag aaatcggcaa ggccagcagc gggcgtggga gatacgggca |
| 1261 | tagaggatgc aaacgctgtc aggggttcag cctctcagat cttgaatcat tttattggat |
| 1321 | ttcctacatt ccttggactg ggtttcgagt ttctcctgaa tctccgtgat ctttgttgcc |
| 1381 | atccagattc tgaattctat gtctatcatt tcagtcattt cagactcatt aagaacattg |
| 1441 | ctggggagat agtgtggtca cttgaaggta aaatactctg gcttttggat gtgtcagatt |
| 1501 | tctttcactg gttcttcctc atctgtgtgg gctgatgttc ctttaatctt tgaagttgct |
| 1561 | gttctttgga tagggctctt tgcttttata ttctctgatg |

SEQ ID NO: 2 Human SIGLEC9 Isoform 1 Amino Acid Sequence
(NP_001185487.1)

| | |
|---|---|
| 1 | mlllllpllw greraegqts klltmqssvt vqeglcvhvp csfsypshgw iypgpvvhgy |
| 61 | wfregantdq dapvatnnpa ravweetrdr fhllgdphtk nctlsirdar rsdagryffr |
| 121 | mekgsikwny khhrlsvnvt althrpnili pgtlesgcpq nltcsvpwac eqgtppmisw |
| 181 | igtsyspldp sttrssvltl ipqpqdhgts ltcqvtfpga svttnktvhl nvsyppqnlt |
| 241 | mtvfqgdgtv stvlgngssl slpeggslrl vcavdavdsn pparlslswr gltlcpsqps |
| 301 | npgvlelpwv hlrdaaeftc raqnplgsqq vylnvslqsk atsgvtqgvv ggagatalvf |
| 361 | lsfcvifvvv rscrkksarp aagvgdtgie danavrgsas qilnhfigfp tflglgfefi |
| 421 | lnlrdlcchp dsefyvyhfs hfrlikniag eivwslegki lwlldvsdff hwfflicvg |

SEQ ID NO: 3 Human SIGLEC9 Transcript Variant 2 cDNA Sequence
(NM_014441.2; CDS: 96-1487)

| | |
|---|---|
| 1 | tagggcctcc tctaagtctt gagcccgcag ttcctgagag aagaaccctg aggaacagac |
| 61 | gttccctcgc ggccctggca cctctaaccc cagacatgct gctgctgctg ctgcccctgc |
| 121 | tctgggggag ggagagggcg gaaggacaga caagtaaact gctgacgatg cagagttccg |
| 181 | tgacggtgca ggaaggcctg tgtgtccatg tgccctgctc cttctcctac ccctcgcatg |
| 241 | gctggattta ccctggccca gtagttcatg gctactggtt ccgggaaggg gccaatacag |
| 301 | accaggatgc tccagtggcc acaaacaacc cagctcgggc agtgtgggag gagactcggg |
| 361 | accgattcca cctccttggg gacccacata ccaagaattg caccctgagc atcagagatg |
| 421 | ccagaagaag tgatgcgggg agatacttct ttcgtatgga gaaaggaagt ataaaatgga |
| 481 | attataaaca tcaccggctc tctgtgaatg tgacagcctt gacccacagg cccaacatcc |
| 541 | tcatcccagg caccctggag tccggctgcc cccagaatct gacctgctct gtgccctggg |
| 601 | cctgtgagca ggggacaccc cctatgatct cctggatagg gacctccgtg tcccccctgg |
| 661 | acccctccac cacccgctcc tcggtgctca ccctcatccc acagccccag gaccatggca |
| 721 | ccagcctcac ctgtcaggtg accttccctg gggccagcgt gaccacgaac aagaccgtcc |
| 781 | atctcaacgt gtcptacccg cctcagaact tgaccatgac tgtcttccaa ggagacggca |
| 841 | cagtatccac agtattggga aatggctcat ctctgtcact cccagagggc cagtctctgc |
| 901 | gcctggtctg tgcagttgat gcagttgaca gcaatccccc tgccaggctg agcctgagct |
| 961 | ggagaggcct gaccctgtgc ccctcacagc cctcaaaccc gggggtgctg gagctgcctt |
| 1021 | gggtgcacct gagggatgca gctgaattca cctgcagagc tcagaaccct ctcggctctc |
| 1081 | agcaggtcta cctgaacgtc tccctgcaga gcaaagccac atcaggagtg actcaggggg |
| 1141 | tgttcggggg agctggagcc acagccctgg tcttcctgtc cttctgcgtc atcttcgttg |
| 1201 | tagtgaggtc ctgcaggaag aaatcggcaa ggccagcagc gggcgtggga gatacgggca |
| 1261 | tagaggatgc aaacgctgtc aggggttcag cctctcaggg gcccgtgact gaaccttggg |
| 1321 | cagaagacag tcccccagac cagcctcccc cagcttctgc ccgctcctca gtgggggaag |
| 1381 | gagagctcca gtatgcatcc ctcagcttcc agatggtgaa gccttgggac tcgcggggac |
| 1441 | aggaggccac tgacaccgag tactcggaga tcaagatcca cagatgagaa actgcagaga |
| 1301 | ctcaccctga ttgagggatc acagcccctc caggcaaggg agaagtcaga ggctgattct |
| 1561 | tgtagaatta acagccctca acgtgatgag ctatgataac actatgaatt atgtgcagag |
| 1621 | tgaaaagcac acaggcttta gagtcaaagt atctcaaacc tgaatccaca ctgtgccctc |
| 1681 | ccttttattt ttttaactaa aagacagaca aattcctaaa aaaaaaaaaa aaaaaaa |

SEQ ID NO: 4 Human SIGLEC9 Isoform 2 Amino Acid Sequence (NP_055236.1)

| | |
|---|---|
| 1 | mlllllpllw greraegqts klltmqssvt vqeglcvhvp csfsypshgw iypgpvvhgy |
| 61 | wfregantdq dapvatnnpa ravweetrdr fhllgdphtk nctlsirdar rsdagryffr |
| 121 | mekgsikwny khhrlsvnvt althrpnili pgtlesgcpq nltcsvpwac eqgtppmisw |
| 181 | igtsvspldp sttrssvltl ipqpqdhgts ltcqvtfpga svttnktvhl nvsyppqnlt |
| 241 | mtvfqgdgtv stvlgngssl slpegqslrl vcavdavdsn pparlslswr gltlcpsqps |
| 301 | npgvlelpwv hlrdaaeftc raqnplgsqq vylnvslqsk atsgvtqgvv ggagatalvf |
| 361 | lsfcvifvvv rscrkksarp aagvgdtgie danavrgsas qgpltepwae dsppdqpppa |
| 421 | sarssvgege lqyaslsfqm vkpwdsrgqe atdteyseik ihr |

SEQ ID NO: 5 Mouse SIGLEC9 cDNA Sequence (NM_031181.2; CDS: 30-1433)

| | |
|---|---|
| 1 | agagcctgga gagacagttt tagctggaca tgctgctgtt gctgctgctg ctgctgctct |
| 61 | gggggataaa gggtgtggag ggtcagaacc cccaagaggt tttcaccctg aatgtggaaa |
| 121 | ggaaggtggt ggtgcaggag ggcctgtgtg tccttgtgcc ctgtaacttc tcctatctca |
| 181 | agaagaggtt gactgactgg actgactcag acccagttca tggattctgg tacagggaag |
| 241 | gaaccgacag acgcaaagat tccatcgtgg ccacaaataa cccaattcgt aaagcagtga |
| 301 | aggaaacccg gaatcgattc ttcctgctcg gagacccttg gaggaatgac tgctccctga |
| 361 | acatcagaga gatcagaaag aaggatgcgg ggttatactt ctttcgcctg gagcgtggaa |
| 421 | aaacaaagta taattacatg tgggacaaga tgactctggt tgtaacagcc ctcactaaca |
| 481 | ccccccaaat tcttctcccg gagacgcttg aagctggcca tcccagcaac ctgacctgct |
| 541 | ctgtgccttg ggactgtggg tggacggcac ctcccatctt ctcctggact ggtacctctg |
| 601 | tgtcattttt gagcaccaac actacgggtt cctcagtgct aaccatcacc cctcagcctc |
| 661 | aggaccatgg caccaacctc acttgtcagg tgaccctgcc tggaactaat gtgtccacaa |
| 721 | gaatgaccat ccgtctcaac gtgtcatatg ctccaaagaa tctgactgtg accatctatc |
| 781 | aaggagctga ctcagtttcc acaatcctga agaatggctc atctcttcct atctgtgagg |
| 841 | gccagtcact gcgtctcatc tgcagcaccg agagctatcc ccctgcgaac ctaagctggt |
| 901 | cctgggataa cctgaccctg tgcccatcaa agttgtccaa gcccgggctc ctggagctgt |
| 961 | ttccagtgca tcttaagcac ggaggagtgt atacctgcca agctcaacat gccctgggct |
| 1021 | cccaacacat ttccttgagc ctgtctccac agagcagtgc aactttatct gaaatgatga |
| 1081 | tggggacctt tgtgggttca ggagtcacag ccctcctttt cctgtctgtc tgcatcctcc |
| 1141 | tcctggcagt aagatcctac aggaggaaac cagccaggcc agctgtggta gctccgcacc |
| 1201 | cagatgccct caaggtctca gtttctcaga atcccctggt tgaatcccag gcagatgaca |
| 1261 | gctctgagcc cctgccttcc atacttgagg cggccccctc ctccacagag gaagagatac |
| 1321 | attatgcgac cctcagcttt cacgagatga agcccatgaa cctgtggggg caacaggaca |
| 1381 | ctaccacgga gtactcagag ataaagtttc cacaaaggac cgcatggcca tgaccgtggc |
| 1441 | tggagaaagc atggtggccc caggaggctg gctctggaga aggaaagagt cacctaggtt |
| 1501 | gacatgtgat acacagggct tcagagccat tccttctgtg acacaggcgt tctgtgctgc |
| 1561 | ccatgcccct gtgctgccct gtccacacaa ctgctattgt gccctgggaa tcataagctt |
| 1621 | gaccttttta ctcctcttct ccccttcccc tccttccccg ccccctcttc tcctcctcct |
| 1681 | ctctcccctc tcctcccctc ttccttttgt tttttccaag acagggtttc tctgtgtagc |
| 1741 | cttggctgtc ctagaacttg ctctgcagac cagactgccc ttgaactctt ctttccaagt |
| 1801 | gctgggatta aagatgtgca ccaccaccca gcacttgact ttattgtaa |

SEQ ID NO: 6 Mouse SIGLEC9 Amino Acid Sequence (NP_112458.2)

| | |
|---|---|
| 1 | mlllllllll wgikgvegqn pqevftlnve rkvvvqeglc vlvpcnfsyl kkrltdwtds |
| 61 | dpvhgfwyre gtdrrkdsiv atnnpirkav ketrnrffll gdpwrndcsl nireirkkda |
| 121 | glyffrlerg ktkynymwdk mtlvvtaltn tpqillpetl eaghpsnltc svpwdcgwta |
| 181 | ppifswtgts vsflstnttg ssvltitpqp qdhgtnltcq vtlpgtnvst rmtirlnvsy |
| 241 | apknltvtiy qgadsvstil kngsslpise gqslrlicst dsyppanlsw swdnltlcps |
| 301 | klskpgllel fpvhlkhggv ytcqaqhalg sqhislslsp qssatlsemm mgtfvgsgvt |
| 361 | allflsvcil llavrsyrrk parpavvaph pdalkvsvsq nplvesqadd sseplpsile |
| 421 | aapssteeei hyatlsfhem kpmnlwgqqd ttteyseikf pqrtawp |

SEQ ID NO: 7 Human VSIG4 Transcript Variant 1 cDNA Sequence
(NM_007268.2; CDS: 128-1327)

| | |
|---|---|
| 1 | ggagtttgag tgagagatat agggaaggaa gggaagtaag cagtcacaga cgctggcggc |
| 61 | caccagaagt ttgagcctct ttggtagcag gaggctggaa gaaaggacag aagtagctct |
| 121 | ggctgtgatg gggatcttac tgggcctgct actcctgggg cacctaacag tggacactta |
| 181 | tggccgtccc atcctggaag tgccagagag tgtaacagga ccttggaaag gggatgtgaa |
| 241 | tcttccctgc acctatgacc ccctgcaagg ctacacccaa gtcttggtga agtggctggt |
| 301 | acaacgtggc tcagaccctg tcaccatctt tctacgtgac tcttctggag accatatcca |
| 361 | gcaggcaaag taccagggcc gcctgcatgt gagccacaag gttccaggag atgtatccct |
| 421 | ccaattgagc accgtggaga tggatgaccg gagccactac acgtgtgaag tcacctggca |
| 481 | gactcctgat ggcaaccaag tcgtgagaga taagattact gagctccgtg tccagaaact |
| 541 | ctctgtctcc aagcccacag tgacaactgg cagcggttat ggcttcacgg tgccccaggg |
| 601 | aatgaggatt agccttcaat gccaggctcg gggttctcct cccatcagtt atatttggta |
| 661 | taagcaacag actaataacc aggaacccat caaagtagca accctaagta ccttactctt |
| 721 | caagcctgcg gtgatagccg actcaggctc ctatttctgc actgccaagg gccaggttgg |
| 781 | ctctgagcag cacagcgaca ttgtgaagtt tgtggtcaaa gactcctcaa agctactcaa |
| 841 | gaccaagact gaggcaccta caaccatgac ataccccttg aaagcaacat ctacagtgaa |
| 901 | gcagtcctgg gactggacca ctgacatgga tggctacctt ggagagacca gtgctgggcc |
| 961 | aggaaagagc ctgcctgtct ttgccatcat cctcatcatc tccttgtgct gtatggtggt |
| 1021 | ttttaccatg gcctatatca tgctctgtcg gaagacatcc caacaagagc atgtctacga |
| 1081 | agdagccagg gcacatgcca gagaggccaa cgactctgga gaaaccatga gggtggccat |
| 1141 | cttcgcaagt ggctgctcca gtgatgagcc aacttcccag aatctgggca acaactactc |
| 1201 | tgatgagccc tgcataggac aggagtacca gatcatcgcc cagatcaatg gcaactacgc |
| 1261 | ccgcctgctg gacacagttc ctctggatta tgagtttctg gccactgagg gcaaaagtgt |
| 1321 | ctgttaaaaa tgccccatta ggccaggatc tgctgacata attgcctagt cagtccttgc |
| 1381 | cttctgcatg gccttcttcc ctgctacctc tcttcctgga tagcccaaag tgtccgccta |
| 1441 | ccaacactgg agccgctggg agtcactggc tttgccctgg aatttgccag atgcatctca |
| 1501 | agtaagccag ctgctggatt tggctctggg cccttctagt atctctgccg ggggcttctg |
| 1561 | gtactcctct ctaaatacca gaggccagat gcccatagca ctaggacttg gtcatcatgc |
| 1621 | ctacagacac tattcaactt tggcatcttg ccaccagaag acccgaggga ggctcagctc |
| 1681 | tgccagctca gaggaccagc tatatccagg atcatttctc tttcttcagg gccagacagc |
| 1741 | ttttaattga aattgttatt tcacaggcca gggttcagtt ctgctcctcc actataagtc |
| 1801 | taatgttctg actctctcct ggtgctcaat aaatatctaa tcataacagc aaaaaaaaaa |
| 1861 | aaaaaaaaa |

SEQ ID NO: 8 Human VSIG4 Isoform 1 Amino Acid Sequence (NP_909199.1)

| | |
|---|---|
| 1 | mgillgllll ghltvdtygr pilevpesvt gpwkgdvnlp ctydplqgyt qvlvkwlvqr |
| 61 | gsdpvtiflr dssgdhiqqa kyqgrlhvsh kvpqdvslql stlemddrsh ytcevtwqtp |
| 121 | dgnqvvrdki telrvqklsv skptvttgsg ygftvpqgmr islqcqargs ppisyiwykq |
| 181 | qtnnqepikv atlstllfkp aviadsgsyf ctakgqvgse qhsdivkfvv kdsskllktk |
| 241 | teapttmtyp lkatstvkqs wdwttdmdgy lgetsagpgk slpvfaiill islccmvvft |
| 301 | mayimlcrkt sqqehvyeaa rahareands getmrvaifa sgcssdepts qnlgnnysde |
| 361 | pcigqeyqii aqingnyarl ldtvpldyef lategksvc |

SEQ ID NO: 9 Human VSIG4 Transcript Variant 2 cDNA Sequence
(NM_001100431.1; CDS: 128-1045)

| | |
|---|---|
| 1 | ggagtttgag tgagagatat agggaaggaa gggaagtaag cagtcacaga cgctggcggc |
| 61 | caccagaagt ttgagcctct ttggtagcag gaggctggaa gaaaggacag aagtagctct |
| 121 | ggctgtgatg gggatcttac tgggcctgct actcctgggg cacctaacag tggacactta |
| 181 | tggccgtccc atcctggaag tgccagagag tgtaacagga ccttggaaag gggatgtgaa |
| 241 | tcttccctgc acctatgacc ccctgcaagg ctacacccaa gtcttggtga agtggctggt |
| 301 | acaacgtggc tcagaccctg tcaccatctt tctacgtgac tcttctggag accatatcca |
| 361 | gcaggcaaag taccagggcc gcctgcatgt gagccacaag gttccaggag atgtatccct |
| 421 | ccaattgagc accctggaga tggatgaccg gagccactac acgtgtgaag tcacctggca |
| 481 | gactcctgat ggcaaccaag tcgtgagaga taagattact gagctccgtg tccagaaaca |
| 541 | ctcctcaaag ctactcaaga ccaagactga ggcacctaca accatgacat accccttgaa |
| 601 | agcaacatct acagtgaagc agtcctggga ctggaccact gacatggatg gctaccttgg |
| 661 | agagaccagt gctgggccag gaaagagcct gcctgtcttt gccatcatcc tcatcatctc |
| 721 | cttgtgctgt atggtggttt ttaccatggc ctatatcatg ctctgtcgga agacatccca |
| 781 | acaagagcat gtctacgaag cagccagggc acatgccaga gaggccaacg actctggaga |
| 841 | aaccatgagg gtggccatct tcgcaagtgg ctgctccagt gatgagccaa cttcccagaa |
| 901 | tctagggcaa aactactctg atgagccctg cataggacag gagtaccaga tcatcgccca |
| 961 | gatcaatggc aactacgccc gcctgctgga cacagttcct ctggattatg agtttctggc |
| 1021 | cactgagggc aaaagtgtct gttaaaaatg ccccattagg ccaggatctg ctgacataat |
| 1081 | tgcctagtca gtccttgcct tctgcatggc cttcttccct gctacctctc ttcctggata |
| 1141 | gcccaaagtg tccgcctacc aacactggag ccgctgggag tcactggctt tgccctggaa |
| 1201 | tttgccagat gcatctcaag taagccagct gctggatttg gctctgggcc cttctagtat |
| 1261 | ctctgccggg ggcttctggt actcctctct aaataccaga gggaagatgc ccatagcact |
| 1321 | aggacttggt catcatgcct acagacacta ttcaactttg gcatcttgcc accagaagac |
| 1381 | ccgagggagg ctcagctctg ccagctcaga ggaccagcta tatccaggat catttctctt |
| 1441 | tcttcagggc cagacagctt ttaattgaaa ttgttatttc acaggccagg gttcagttct |
| 1501 | gctcctccac tataagtcta atgttctgac tctctcctgg tgctcaataa atatctaatc |
| 1561 | ataacagcaa aaaaaaaaaa aaaaaa |

SEQ ID NO: 10 Human VSIG4 Isoform 2 Amino Acid Sequence (NP_001093901.1)

| | |
|---|---|
| 1 | mgillgllll ghltvdtygr pilevpesvt gpwkgdvnlp ctydplqgvt qvlvkwlvqr |
| 61 | gsdpvtiflr dssgdhiqqa kyqgrlhvsh kvpgdvslql stlemddish ytcevtwqtp |
| 121 | dgnqvvrdki telrvqkhss kllktkteap ttmtyplkat stvkqswdwt tdmdgylget |
| 181 | sagpgkslpv faiiliislc cmvvftmayi mlcrktsqqe hvyeaaraha reandsgetm |
| 241 | rvaifasgcs sdeptsqnlg nnysdepcig qeyqiiaqin gnyarlldtv pldyeflate |
| 301 | gksvc |

SEQ ID NO: 11 Human VSIG4 Transcript Variant 3 cDNA Sequence
(NM_001184831.1; CDS: 128-811)

| | |
|---|---|
| 1 | ggagtttgag tgagagatat agggaaggaa gggaagtaag cagtcacaga cgctggcggc |
| 61 | caccagaagt ttgagcctct ttggtagcag gaggctggaa gaaaggacag aagtagctct |
| 121 | ggctgtgatg gggatcttac tgggcctgct actcctgggg cacctaacag tggacactta |
| 181 | tggccgtccc atcctggaag tgccagagag tgtaacagga ccttggaaag gggatgtgaa |
| 241 | tcttccctgc acctatgacc ccctgcaagg ctacacccaa gtcttggtga agtggctggt |
| 301 | acaacgtggc tcagaccctg tcaccatctt tctacgtgac tcttctggag accatatcca |
| 361 | gcaggcaaag taccagggcc gcctgcatgt gagccacaag gttccaggag atgtatccct |
| 421 | ccaattgagc accctggaga tggatgaccg gagccactac acgtgtgaag tcacctggca |
| 481 | gactcctgat ggcaaccaag tcgtgagaga taagattact gagctccgtg tccagaaaca |
| 541 | ctcctcaaag ctactcaaga ccaagactga ggcacctaca accatgacat accccttgaa |
| 601 | agcaacatct acagtgaagc agtcctggga ctggaPcact gacatggatg gctaccttgg |
| 661 | accgaccagt gctgggccag gaaagagcct gcctgtcttt gccatcatcc tcatcatctc |
| 721 | cttgtgctgt atggtggttt ttaccatggc ctatatcatg ctctgtcgga agacatccca |
| 781 | acaagagcat gtctacgaag cagccaggta agaaagtctc tcctcttcca tttttgaccc |
| 841 | cgtccctgcc ctcaattttg attactggca ggaaatgtgg aggaaggggg gtgtggcaca |
| 901 | gacccaatcc taaggccgga ggccttcagg gtcaggacat agctgccttc cctctctcag |
| 961 | gcaccttctg aggttgtttt ggccctctga acacaaagga taatttagat ccatctgcct |
| 1021 | tctgcttcca gaatccctgg gtggtaggat cctgataatt aattggcaag aattgaggca |
| 1081 | gaagggtggg aaaccaggac cacagcccca agtcccttct tatgggtggt gggctcttgg |
| 1141 | gccatagggc acatgccaga gaggccaacg actctggaga aaccatgagg gtggccatct |
| 1201 | tcgcaagtgg ctgctccagt gatgagccaa cttcccagaa tctgggcaac aactactctg |
| 1261 | atgagccctg cataggacag gagtaccaga tcatcgccca gatcaatggc aactacgccc |
| 1321 | gcctgctgga cacagttcct ctggattatg agtttctggc cactgagggc aaaagtgtct |
| 1381 | gttaaaaatg ccccattagg ccaggatctg ctgacataat tgcctagtca gtccttgcct |
| 1441 | tctgcatggc cttcttccct gctacctctc ttcctggata gcccaaagtg tccgcctacc |
| 1501 | aacactggag ccgctgggag tcactggctt tgccctggaa tttgccagat gcatctcaag |
| 1561 | taagccagct gctggatttg gctctgggcc cttctagtat ctctgccggg ggcttctggt |
| 1621 | actcctctct aaataccaga gggaagatgc ccatagcact aggacttggt catcatgcct |
| 1681 | acagacacta ttcaactttg gcatcttgcc accagaagac ccgagggagg ctcagctctg |
| 1741 | ccagctcaga ggaccagcta tatccaggat catttctctt tcttcagggc cagacagctt |
| 1801 | ttaattgaaa ttgttatttc acaggccagg gttcagttct gctcctccac tataagtcta |
| 1861 | atgttctgac tctctcctgg tgctcaataa atatctaatc ataacagcaa aaaaaaaaaa |
| 1921 | aaaaaaa |

SEQ ID NO: 12 Human VSIG4 Isoform 3 Amino Acid Sequence (NP_001171760.1)

| | |
|---|---|
| 1 | mgillgllll ghltvdtygr pilevpesvt gpwkgdynlp ctydplqgyt qvlvkwlvqr |
| 61 | gsdpvtiflr dssgdhiqqa kyqgrlhvsh kvpgdyslql stlemddrsh ytcevtwqtp |
| 121 | dgnqvvrdki telrvqkhss kllktkteap ttmtyplkat stvkqswdwt tdmdgylget |
| 181 | sagpgkslpv faiiliislc cmvvftmayi mlcrktsqqe hvyeaar |

SEQ ID NO: 13 Human VSIG4 Transcript Variant 4 cDNA Sequence
(NM_001184830.1; CDS: 128-1093)

| | |
|---|---|
| 1 | ggagtttgag tgagagatat agggaaggaa gggaagtaag cagtcacaga cgctggcggc |
| 61 | caccagaagt ttgagcctct ttggtagcag gaggctggaa gaaaggacag aagtagctct |
| 121 | ggctgtgatg gggatcttac tgggcctgct actcctgggg cacctaacag tggacactta |
| 181 | tggccgtccc atcctggaag tgccagagag tgtaacagga ccttggaaag gggatgtgaa |
| 241 | tcttccctgc acctatgacc ccctgcaagg ctacacccaa gtcttggtga agtggctggt |
| 301 | acaacgtggc tcagaccctg tcaccatctt tctacgtgac tcttctggag accatatcca |
| 361 | gcaggcaaag taccagggcc gcctgcatgt gagccacaag gttccaggag atgtatccct |
| 421 | ccaattgagc accctggaga tggatgaccg gagccactac acgtgtgaag tcacctggca |
| 481 | gactcctgat ggcaaccaag tcgtgagaga taagattact gagctccgtg tccagaaact |
| 541 | ctctgtctcc aagcccacag tgacaactgg cagcggttat ggcttcacgg tgccccaggg |
| 601 | aatgaggatt agccttcaat gccaggctcg gggttctcct cccatcagtt atatttggta |
| 661 | taagcaacag actaataacc aggaacccat caaagtagca accctaagta ccttactctt |
| 721 | caagcctgcg gtgatagccg actcaggctc ctatttctgc actgccaagg gccaggttgg |
| 781 | ctctgagcag cacagcgaca ttgtgaagtt tgtggtcaaa gactcctcaa agctactcaa |
| 841 | gaccaagact gaggcaccta caaccatgac ataccccttg aaagcaacat ctacagtgaa |
| 901 | gcagtcctgg gactggacca ctgacatgga tggctacctt ggagagacca gtgctgggcc |
| 961 | aggaaagagc ctgcctgtct ttgccatcat cctcatcatc tccttgtgct gtatggtggt |
| 1021 | ttttaccatg gcctatatca tgctctgtcg gaagacatcc caacaagagc atgtctacga |
| 1081 | agcagccagg taagaaagtc tctcctcttc catttttgac cccgtccctg ccctcaattt |
| 1141 | tgattactgg caggaaatgt ggaggaaggg gggtgtggca cagacccaat cctaaggccg |
| 1201 | gaggccttca gggtcaggac atagctgcct tccctctctc aggcaccttc tgaggttgtt |
| 1261 | ttggccctct gaacacaaag gataatttag atccatctgc cttctgcttc cagaatccct |
| 1321 | gggtggtagg atcctgataa ttaattggca agaattgagg cagaagggtg ggaaaccagg |
| 1381 | accacagccc caagtccctt cttatgggtg gtgggctctt gggccatagg gcacatgcca |
| 1441 | gagaggccaa cgactctgga gaaaccatga gggtggccat cttcgcaagt ggctgctcca |
| 1501 | gtgatgagcc aacttcccag aatctgggca acaactactc tgatgagccc tgcataggac |
| 1561 | aggagtacca gatcatcgcc cagatcaatg gcaactacgc ccgcctgctg gacacagttc |
| 1621 | ctctggatta tgagtttctg gccactgagg gcaaaagtgt ctgttaaaaa tgccccatta |
| 1681 | ggccaggatc tgctgacata attgcctagt cagtccttgc cttctgcatg gccttcttcc |
| 1741 | ctgctacctc tcttcctgga tagcccaaag tgtccgccta ccaacactgg agccgctggg |
| 1801 | agtcactggc tttgccctgg aatttgccag atgcatctca agtaagccag ctgctggatt |
| 1861 | tggctctggg cccttctagt atctctgccg ggggcttctg gtactcctct ctaaatacca |
| 1921 | gagggaagat gcccatagca ctaggacttg gtcatcatgc ctacagacac tattcaactt |
| 1981 | tggcatcttg ccaccagaag acccgaggga ggctcagctc tgccagctca gaggaccagc |
| 2041 | tatatccagg atcatttctc tttcttcagg gccagacagc ttttaattga aattgttatt |
| 2101 | tcacaggcca gggttcagtt ctgctcctcc actataagtc taatgttctg actctctcct |
| 2161 | ggtgctcaat aaatatctaa tcataacagc aaaaaaaaaa aaaaaaaaa |

SEQ ID NO: 14 Human VSIG4 Isoform 4 Amino Acid Sequence (NP_001171759.1)

| | |
|---|---|
| 1 | mgillgllll ghltvdtygr pilevpesvt gpwkgdynlp ctydplqgyt gvlvkwlvgr |
| 61 | gsdpvtiflr dssgdhiqqa kyqgrlhvsh kvpgdvslql stlemddrsh ytcevtwqtp |
| 121 | dgnqvvrdki telrvqklsv skptvttgsg ygftypqgmr islqcqargs ppisyiwykq |
| 181 | gtnnqepikv atlstllfkp aviadsgsyf ctakgqvgse qhsdivkfvv kdsskllktk |
| 241 | teapttmtyp lkatstvkqs wdwttdmdgy lgetsagpgk slpvfaiili islccmvvft |
| 301 | mayimlcrkt sqqehvyeaa a |

SEQ ID NO: 15 Human VSIG4 Transcript Variant 5 cDNA Sequence
(NM_001257403.1; CDS: 128-1171)

| | |
|---|---|
| 1 | ggagtttgag tgagagatat agggaaggaa gggaagtaag cagtcacaga cgctggcggc |
| 61 | caccagaagt ttgagcctct ttggtagcag gaggctggaa gaaaggacag aagtagctct |
| 121 | ggctgtgatg gggatcttac tgggcctgct actcctgggg cacctaacag tggacactta |
| 181 | tggccgtccc atcctggaag tgccagagag tgtaacagga ccttggaaag gggatgtgaa |
| 241 | tcttccgtgc acctatgacc ccctgcaagg ctacacccaa gtcttggtga agtggctggt |
| 301 | acaacgtggc tcagaccctg tcaccatctt tctacgtgac tcttctggag accatatcca |
| 361 | gcaggcaaag taccagggcc gcctgcatgt gagccacaag gttccaggag atgtatccct |
| 421 | ccaattgagc accctggaga tggatgaccg gagccactac acgtgtgaag tcacctggca |
| 481 | gactcctgat ggcaaccaag tcgtgagaga taagattact gagctccgtg tccagaaact |
| 541 | ctctgtctcc aagcccacag tgacaactgg cagcggttat ggcttcacgg tgccccaggg |
| 601 | aatgaggatt agccttcaat gccaggctcg gggttctcct cccatcagtt atatttggta |
| 661 | taagcaacag actaataacc aggaacccat caaagtagca accctaagta ccttactctt |
| 721 | caagcctgcg gtgatagccg actcaggctc ctatttctgc actgccaagg gccaggttgg |
| 781 | ctctgagcag cacagcgaca ttgtgaagtt tgtggtcaaa gactcctcaa agctactcaa |
| 841 | gaccaagact gaggcaccta caaccatgac ataccccttg aaagcaacat ctacagtgaa |
| 901 | gcagtcctgg gactggacca ctgacatgga tggctacctt ggagagacca gtgctgggcc |
| 961 | aggaaagagc ctgcctgtct ttgccatcat cctcatcatc tccttgtgct gtatggtggt |
| 1021 | ttttaccatg gcctatatca tgctctgtcg gaagacatcc caacaagagc atgtctacga |
| 1081 | agcagccagc ccaaagtgtc cgcctaccaa cactggagcc gctgggagtc actggctttg |
| 1141 | ccctggaatt tgccagatgc atctcaagta agccagctgc tggatttggc tctgggccct |
| 1201 | tctagtatct ctgccggggg cttctggtac tcctctctaa ataccagagg gaagatgccc |
| 1261 | atagcactag gacttggtca tcatgcctac agacactatt caactttggc atcttgccac |
| 1321 | cagaagaccc gagggaggct cagctctgcc agctcagagg accagctata tccaggatca |
| 1381 | tttctctttc ttcagggcca gacagctttt aattgaaatt gttatttcac aggccagggt |
| 1441 | tcagttctgc tcctccacta taagtctaat gttctgactc tctcctggtg ctcdataaat |
| 1501 | atctaatcat aacagcaaaa aaaaaaaaaa aaaaa |

SEQ ID NO: 16 Human VSIG4 Isoform 5 Amino Acid Sequence (NP_001244332.1)

| | |
|---|---|
| 1 | mgillgllll ghltvdtygr pilevpesvt gpwkgdvnlp ctydplqgyt qvlvkwlvqr |
| 61 | gsdpvtiflr dssgdhiqqa kyqgrlhvsh kvpgdvslql stlemddrsh ytcevtwqtp |
| 121 | dgnqvvrdki telrvqklsv skptvttgsg vgftvpqgmr islqcqargs ppisyiwykq |
| 181 | qtnnqepikv atlstllfkp aviadsgsyf ctakgqvgse qhsdivkfvv kdsskllktk |
| 241 | teapttmtyp lkatstvkqs wdwttdmdgy lgetsagpgk slpvfaiili islccmvvft |
| 301 | mayimlcrkt sqqehvyeaa spkcpptntg aagshwlcpg icqmhlk |

SEQ ID NO: 17 Mouse VSIG4 cDNA Sequence (NM_177789.4; CDS: 71-913)

| | |
|---|---|
| 1 | agctaccagc acttccaggt tcttcagcag caagaggatg gaaggatgaa tagaagtagc |
| 61 | ttcaaatagg atggagatct catcaggctt gctgttcctg ggccacctaa tagtgctcac |
| 121 | ctatggccac cccaccctaa aaacacctga gagtgtgaca gggacctgga aaggagatgt |
| 181 | gaagattcag tgcatctatg atcccctgag aggctacagg caagttttgg tgaaatggct |
| 241 | ggtaagacac ggctctgact ccgtcaccat cttcctacgt gactccactg gagaccatat |
| 301 | ccagcaggca aagtacagag gccgcctgaa agtgagccac aaagttccag gagatgtgtc |
| 361 | cctccaaata aataccctgc agatggatga caggaatcac tatacatgtg aggtcacctg |
| 421 | gcagactcct gatggaaacc aagtaataag agataagatc attgagctcc gtgttcggaa |
| 481 | atataatcca cctagaatca atactgaagc acctacaacc ctgcactcct ctttggaagc |
| 541 | aacaactata atgagttcaa cctctgactt gaccactaat gggactggaa aacttgagga |
| 601 | gaccattgct ggttcaggga ggaacctgcc aatctttgcc ataatcttca tcatctccct |
| 661 | ttgctgcata gtagctgtca ccatacctta tatcttgttc cgctgcagga cattccaaca |
| 721 | agagtatgtc tatggagtga gcagggtgtt tgccaggaag acaagcaact ctgaagaaac |
| 781 | cacaagggtg actaccatcg caactgatga accagattcc caggctctga ttagtgacta |
| 841 | ctctgatgat ccttgcctca gccaggagta ccaaataacc atcagatcaa caatgtctat |
| 901 | tcctgcctgc tgaacacagt ttccagaaac taagaagttc ttgctactga agaaaataac |
| 961 | atctgctaaa atgcccctac taagtcaagg tctactggcg taattacctg ttacttattt |
| 1021 | actacttgcc ttcaacatag ctttctccct ggcttccttt cttcttagac aacctaaagt |
| 1081 | atctatctag tctgccaatt ctggggccat tgagaaatcc tgggtttggc taagaatata |
| 1141 | ctacatgcac ctcaagaaat ctagcttctg ggcttcaccc agaacaattt tcttcctagg |
| 1201 | gccttcacaa ctcttctcca aacagcagag aaattccata gcagtagagg ttctttatca |
| 1261 | tgcctccaga cagcgtgagt ctcagtccta caaactcaga caagcacatg ggtctaggat |
| 1321 | tactcctctt tctctagggc cagatgactt ttaattgata ttactattgc tacattatga |
| 1381 | atctaatgca catgtattct tttgttgtta ataaatgttt aatcatgaca tc |

SEQ ID NO: 18 Mouse VSIG4 Amino Acid Sequence (NP_808457.1)

| | |
|---|---|
| 1 | meissgllfl ghlivltygh ptlktpesvt gtwkgdvkiq ciydplrgyr qvlvkwlvrh |
| 61 | gsdsvtiflr dstgdhiqqa kyrgrlkvsh kvpqdvslqi ntlqmddrnh ytcevtwqtp |
| 121 | dgnqvirdki ielrvrkynp printeaptt lhssleatti msstsdlttn gtgkleetia |
| 181 | gsgrnlpifa iifiislcci vavtipyilf rcrtfqqeyv ygvsrvfark tsnseettrv |
| 241 | ttiatdepds qalisdysdd pclsqeyqit irstmsipac |

SEQ ID NO: 19 Human CD74 Transcript Variant 1 cDNA Sequence
(NM_001025159.2; CDS: 188-1078)

| | |
|---|---|
| 1 | ctgcctgggg agcccccccg ccccacatcc tgcccggcaa aaggcagctt caccaaagtg |
| 61 | gggtatttcc agcctttgta gctttcactt ccacatctac caagtgggcg gagtggcctt |
| 121 | ctgtggacga atcagattcc tctccagcac cgactttaag aggcgagccg gggggtcagg |
| 181 | gtcccagatg cacaggagga gaaagcagga ctgtcgggaa gatcagaagc cagtcatgga |
| 241 | tgaccagcgc gaccttatct ccaacaatga gcaactgccc atgctgggcc ggcgccctgg |
| 301 | ggccccggag agcaagtgca gccggggagc cctgtacaca ggcttttcca tcctggtgac |
| 361 | tctgctcctc gctggccagg ccaccaccgc ctacttcctg taccagcagc agggccggct |
| 421 | ggacaaactg acagtcacct cccagaacct ggagctggag aacctgcgca tgaagcttcc |
| 481 | caagcctccc aagcctgtga gcaagatgcg catggccacc ccgctgctga tgcaggcgct |
| 541 | gcccatggga gccctgcccc aggggcccat gcagaatgcc accaagtatg gcaacatgac |
| 601 | agaggaccat gtgatgcacc tgctccagaa tgctgacccc ctgaaggtgt acccgccact |
| 661 | gaaggggagc ttcccggaga acctgagaca ccttaagaac accatggaga ccatagactg |
| 721 | gaaggtcttt gagagctgga tgcaccattg gctcctgttt gaaatgagca ggcactcctt |
| 781 | ggagcaaaag cccactgacg ctccaccgaa agtactgacc aagtgccagg aagaggtcag |
| 841 | ccacatccct gctgtccacc cgggttcatt caggcccaag tgcgacgaga acggcaacta |
| 901 | tctgccactc cagtgctatg ggagcatcgg ctactgctgg tgtgtcttcc ccaacggcac |
| 961 | ggaggtcccc aacaccagaa gccgcgggga ccataactgc agtgagtcac tggaactgga |
| 1021 | ggacccgtct tctgggctgg gtgtgaccaa gcaggatctg ggcccagtcc ccatgtgaga |
| 1081 | gcagcagagg cggtcttcaa catcctgcca gccccacaca gctacagctt tcttgctcpc |
| 1141 | ttcagccccc agcccctccc ccatctccca ccctgtacct catcccatga gaccctggtg |
| 1201 | cctggctctt tcgtcaccct tggacaagac aaaccaagtc ggaacagcag ataacaatgc |
| 1261 | agcaaggccc tgctgcccaa tctccatctg tcaacagggg cgtgaggtcc caggaagtgg |
| 1321 | ccaaaagcta gacagatccc cgttcctgac atcacagcag cctccaacac aaggctccaa |
| 1381 | gacctaggct catggacgag atgggaaggc acagggagaa gggataaccc tacacccaga |
| 1441 | ccccaggctg gacatgctga ctgtcctctc ccctccagcc tttggccttg gcttttctag |
| 1501 | cctatttacc tgcaggctga gccactctct tccctttccc cagcatcact ccccaaggaa |
| 1561 | gagccaatgt tttccaccca taatcctttc tgccgacccc tagttccctc tgctcagcca |
| 1621 | agcttgttat cagctttcag ggccatggtt cacattagaa taaaaggtag taattagaac |
| 1681 | aaaaaaaaaa aaaaaaaa |

SEQ ID NO: 20 Human CD74 Isoform A Amino Acid Sequence (NP_001020330.1)

| | |
|---|---|
| 1 | mhrrrsrscr edqkpvmddq rdlisnneql pmlgrrpgap eskcsrgaly tgfsilvtll |
| 61 | lagqattayf lyqqqgrldk ltvtsqnlql enlrmklpkp pkpvskmrma tpllmqalpm |
| 121 | galpqgpmqn atkygnmted hvmhllqnad plkvypplkg sfpenlrhlk ntmetidwkv |
| 181 | feswmhhwll femsrhsleq kptdappkvl tkcqeevshi pavhpgsfrp kcdengnylp |
| 241 | lqcygsigyc wcvfpngtev pntrsrghhn csesleledp ssglgvtkqd lgpvpm |

SEQ ID NO: 21 Human CD74 Transcript Variant 2 cDNA Sequence (NM_004355.3;
CDS: 188-886)

| | |
|---|---|
| 1 | ctgcctgggg agccccggcg ccccacatcc tgccccgcaa aaggcagctt caccaaagtg |
| 61 | gggtatttcc agcctttgta gctttcactt ccacatctac caagtgggcg gagtggcctt |
| 121 | ctgtggacga atcagattcc tctccagcac cgactttaag aggcgagccg gggggtcagg |
| 181 | gtcccagatg cacaggagga gaagcaggag ctgtcgggaa gatcagaagc cagtcatgga |
| 241 | tgaccagcgc gaccttatct ccaacaatga gcaactgccc atgctgggcc ggcgccctgg |
| 301 | ggccccggag agcaagtgca gccgcggagc cctgtacaca ggcttttcca tcctggtgac |
| 361 | tctgctcctc gctggccagg ccaccaccgc ctacttcctg taccaggagc agggccggct |
| 421 | ggacaaactg acagtcacct cccagaacct ggagctggag aacctgcgca tgaagcttcc |
| 481 | caagcctccc aagcctgtga gcaagatgcg catggccacc ccgctgctga tgcaggcgct |
| 541 | gcccatggga gccctgcccc aggggcccat gcagaatgcc accaagtatg gcaacatgac |
| 601 | agaggaccat gtgatgcacc tgctccagaa tgctgacccc ctggaggtgt acccgccact |
| 661 | gaaggggagc ttcccggaga acctgagaca ccttaagaac accatggaga ccatagactg |
| 721 | gaaggtcttt gagagctgga tgcaccattg gctcctgttt gaaatgagca ggcactcctt |
| 781 | ggagcaaaag cccactgacg ctccaccgaa agagtcactg gaactggagg acccgtcttc |
| 841 | tgggctgggt gtgaccaagc aggatctggg cccagtcccc atgtgagagc agcagaggcg |
| 901 | gtcttcaaca tcctgccagc cccacacagc tacagctttc ttgctccctt cagcccccag |
| 961 | cccctccccc atctcccacc ctgtacctca tcccatgaga ccctggtgcc tggctctttc |
| 1021 | gtcacccttg gacaagacaa accaagtcgg aacagcagat aacaatgcag caaggccctg |
| 1081 | ctgcccaatc tccatctgtc aacaggggcg tgaggtccca ggaagtggcc aaaagctaga |
| 1141 | cagatccccg ttcctgacat cacagcagcc tccaacacaa ggctccaaga cctaggctca |
| 1201 | tggacgagat gggaaggcac agggagaagg gataacccta cacccagacc ccaggctgga |
| 1261 | catgctgact gtcctctccc ctccagcctt tggccttggc ttttctagcc tatttacctg |
| 1321 | caggctgagc cactctcttc cctttcccca gcatcactcc ccaaggaaga gccaatgttt |
| 1381 | tccacccata atcctttctg ccgaccccta gttccctctg ctcaggcaag cttgttatca |
| 1441 | gctttcaggg ccatggttca cattagaata aaaggtagta attagaacaa aaaaaaaaaa |
| 1501 | aaaaaa |

SEQ ID NO: 22 Human CD74 Isoform B Amino Acid Sequence (NP_004346.1)

| | |
|---|---|
| 1 | mhrrrsrscr edqkpvmddq rdlisnneql pmlgrrpgap eskcsrgaly tgfsilvtll |
| 61 | lagqattayf lyqqqgrldk ltvtsqnlql enarmklpkp pkpvskmrma tpllmqalpm |
| 121 | galpqgpmqn atkygnmted hvmhllqnad plkvypplkg sfpenlrhlk ntmetidwkv |
| 181 | feswmhhwll femsrhsleq kptdappkes leledpssgl gvtkqdlgpv pm |

SEQ ID NO: 23 Human CD74 Transcript Variant 3 cDNA Sequence
(NM_001025158.2; CDS: 188-670)

| | |
|---|---|
| 1 | ctgcctgggg agcccccccg ccccacatcc tgccccgcaa aaggcagctt caccaaagtg |
| 61 | gggtatttcc agcctttgta gctttcactt ccacatctac caagtgggcg gagtggcctt |
| 121 | ctgtggacga atcagattcc tctccagcac cgactttaag aggcgagccg gggggtcagg |
| 181 | gtcccagatg cacaggagga gaagcaggag ctgtcgggaa gatcagaagc cagtcatgga |
| 241 | tgaccagcgc gaccttatct ccaacaatga gcaactgccc atgctgggcc ggcgccctgg |
| 301 | ggccccggag agcaagtgca gccgcggagc cctgtacaca ggcttttcca tcctggtgac |
| 361 | tctgctcctc gctggccagg ccaccaccgc ctacttcctg taccagcagc agggccggct |
| 421 | ggacaaactg acagtcacct cccagaacct gcagctggag aacctgcgca tgaagcttcc |
| 481 | caagcctccc aagcctgtga gcaagatgcg catggccacc ccgctgctga tgcaggcgct |
| 541 | gcccatggga gccctgcccc aggggcccat gcagaatgcc accaagtatg gcaacatgac |
| 601 | agaggaccat gtgatgcacc tgctccagag tcactggaac tggaggaccc gtcttctggg |
| 661 | ctgggtgtga ccaagcagga tctgggccca gtccccatgt gagagcagca gaggcggtct |
| 721 | tcaacatcct gccagcccca cacagctaca gctttcttgc tcccttcagc ccccagcccc |
| 781 | tcccccatct cccaccctgt acctcatccc atgagaccct ggtgcctggc tctttcgtca |
| 841 | cccttggaca agacaaacca agtcggaaca gcagataaca atgcagcaag gccctgctgc |
| 901 | ccaatctcca tctgtcaaca ggggcgtgag gtcccaggaa gtggccaaaa gctagacaga |
| 961 | tccccgttcc tgacatcaca gcagcctcca acacaaggct ccaagaccta ggctcatggc |
| 1021 | cgagatggga aggcacaggg agaagggata accctacacc cagaccccag gctggacatg |
| 1081 | ctgactgtcc tctcccctcc agcctttggc cttggctttt ctagcctatt tacctgcagg |
| 1141 | ctgagccact ctcttccctt tccccagcat cactccccaa ggaagagcca atgttttcca |
| 1201 | cccataatcc tttctgccga cccctagttc cctctgctca gccaagcttg ttatcagctt |
| 1261 | tcagggccat ggttcacatt agaataaaag gtagtaatta gaacaaaaaa aaaaaaaaaa |
| 1321 | aa |

SEQ ID NO: 24 Human CD74 Isoform C Amino Acid Sequence (NP_001020329.1)

| | |
|---|---|
| 1 | mhmrrrsrcr edqkpvmddq rdlisnneql pmlgrrpgap eskcsrgaly tgfsilvtll |
| 61 | lagqattayf lyqqqgrldk ltvtsqnlql enlrmklpkp pkpvskmrma tpllmqalpm |
| 121 | galpqgpmqn atkygnmted hvmhllqshw nwrtrllgwv |

SEQ ID NO: 25 Mouse CD74 Transcript Variant 1 cDNA Sequence
(NM_001042605.1; CDS: 86-925)

| | |
|---|---|
| 1 | agacacacag cagcagcagc agcagcagca gcagcaacag cagcagcagc agcagcgcct |
| 61 | gtgggaaaaa ctagaggcta gagccatgga tgaccaacgc gacctcatct ctaaccatga |
| 121 | acagttgccc atactgggca accgccctag agagccagaa aggtgcagcc gtggagctct |
| 181 | gtacaccggt gtctctgtcc tggtggctct gctcttggct gggcaggcca ccactgctta |
| 241 | cttcctgtac cagcaacagg gccgcctaga caagctgacc atcacctccc agaacctgca |
| 301 | actggagagc cttcgcatga agcttccgaa atctgccaaa cctgtgagcc agatgcggat |
| 361 | ggctactccc ttgctgatgc gtccaatgtc catggataac atgctccttg ggcctgtgaa |
| 421 | gaacgttacc aagtacggca acatgaccca ggaccatgtg atgcatctgc tcacgaggtc |
| 481 | tggacccctg gagtacccgc agctgaaggg gaccttccca gagaatctga agcatcttaa |
| 541 | gaactccatg gatggcgtga actggaagat cttcgagagc tggatgaagc agtggctctt |
| 601 | gtttgagatg agcaagaact ccctggagga gaagaagccc accgaggctc cacctaaagt |
| 661 | actgaccaag tgccaggaag aagtcagcca catccctgcc gtctacccgg gtgcgttccg |
| 721 | tcccaagtgc gacgagaacg gtaactattt gccactccag tgccacggga gcactggcta |
| 781 | ctgctggtgt gtgttcccca acggcactga ggttcctcac accaagagcc gcgggcgcca |
| 841 | taactgcagt gagccactgg acatggaaga cctatcttct ggcctgggag tgaccaggca |
| 901 | ggaactgggt caagtcaccc tgtgaagaca gaggccagct ctgcacagca gcagcgcccc |
| 961 | ctgctctcct gtgcctcagc ccttcttatg ttccctgatg tcacacccca cttcccgtct |
| 1021 | ccctgcaccc tggggcttga gactggtgtc tgtttcatcg tcccaggaca cggcaaatga |
| 1081 | agtcagaaca gaaggaggac gctggagggc cttgctggct accgctatct aaagggaacc |
| 1141 | cccatttctg acccattagt agtcttgaat gtggggctct gagataaagg cccgcagaca |
| 1201 | gggacaaggg atgccctacc cttaacctag gctggacaca tttgctgcct tctcctcaag |
| 1261 | gaagaagaac ccaagcccct cctcccagta acccctcctc acatcctgcc accccccctc |
| 1321 | aagccccacc ccctttcagg ttcattgctc agccaagctt gtcagcagcc tgtaggatca |
| 1381 | tggttcaagt gacaataaag gaagaaagta gaacaaaaaa aaaaaaaaaa a |

SEQ ID NO: 26 Mouse CD74 Isoform 1 Amino Acid Sequence (NP_001036070.1)

| | |
|---|---|
| 1 | mddqrdlisn heqlpilgnr prepercsrg alytgvsvlv alllagqatt ayflyqqqgr |
| 61 | ldkltitsqn lqleslrmkl pksakpvsqm rmatpllmrp msmdnmllgp vknvtkygnm |
| 121 | tqdhvmhllt rsgpleypql kgtfpenlkh lknsmdgvnw kifeswmkqw llfemsknsl |
| 181 | eekkpteapp kvltkcqeev shipavypga frpkcdengn ylplqchgst gycwcvfpng |
| 241 | tevphtksrg rhncsepldm edlssglgvt rqelgqvtl |

SEQ ID NO: 27 Mouse CD74 Transcript Variant 2 CDNA Sequence (NM_010545.3;
CDS: 6-733)

| | |
|---|---|
| 1 | agacacacag cagcagcagc agcagcagca gcagcaacag cagcaggagc agcagcgcct |
| 61 | gtgggaaaaa ctagaggcta gagccatgga tgaccaacgc gacctcatct ctaaccatga |
| 121 | acagttgccc atactgggca accgccctag agagccagaa aggtgcagcc gtggagctct |
| 181 | gtacaccggt gtctctgtcc tggtggctct gctcttggct gggcaggcca ccactgctta |
| 241 | cttcctgtac cagcaacagg gccgcctaga caagctgacc atcacctccc agaacctgca |
| 301 | actggagagc cttcgcatga agcttccgaa atctgccaaa cctgtgagcc agatgcggat |
| 361 | ggctactccc ttgctgatgc gtccaatgtc catggataac atgctccttg ggcctgtgaa |
| 421 | gaacgttacc aagtacggca acatgaccca ggaccatgtg atgcatctgc tcacgaggtc |
| 481 | tggacccctg gagtacccgc agctgaaggg gaccttccca gagaatctga agcatcttaa |
| 541 | gaactccatg gatggcgtga actggaagat cttcgagagc tggatgaagc agtggctctt |
| 601 | gtttgagatg agcaagaact ccctggagga gaagaagccc accgaggctc cacctaaaga |
| 661 | gccactggac atggaagacc tatcttctgg cctgggagtg accaggcagg aactgggtca |
| 721 | agtcaccctg tgaagacaga ggccagctct gcacagcagc agcgccccct gctctcctgt |
| 781 | gcctcagccc ttcttatgtt ccctgatgtc acaccccact tcccgtctcc ctgcaccctg |
| 841 | gggcttgaga ctggtgtgtg tttcatcgtc ccaggacacg gcaaatgaag tcagaacaga |
| 901 | aggaggacgc tggagggcct tgctggctac cgctatctaa agggaacccc catttctgac |
| 961 | ccattagtag tcttgaatgt ggggctctga gataaaggcc cgcagacagg gacaagggat |
| 1021 | gccctaccct taacctaggc tggacacatt tgctgccttc tcctcaagga agaagaaccc |
| 1081 | aagcccctcc tcccagtaac ccctcctcac atcctgccac cccccctcaa gccccacccc |
| 1141 | ctttcaggtt ccttgctcag ccaagcttgt caggagcctg taggatcatg gttcaagtga |
| 1201 | caataaagga agaaagtaga acaaaaaaaa aaaaaaaaa |

SEQ ID NO: 28 Mouse CD74 Isoform 2 Amino Acid Sequence (NP_034675.1)

| | |
|---|---|
| 1 | mddqrdlisn heqlpilgnr prepercsrg alytgvsvlv alllagqatt avflyqqqgr |
| 61 | ldkltitsqn lqleslrmkl pksakpvsqm rmatpllmrp msmdnmllgp vknvtkygnm |
| 121 | tqdhvmhllt rsgpleypql kgtfpenlkh lknsmdgvnw kifeswmkqw llfemsknsl |
| 181 | eekkpteapp kepldmedls sglgvtrqel gqvtl |

SEQ ID NO: 29 Human CD207 cDNA Sequence (NM_015717.4; CDS: 48-1034)

| | |
|---|---|
| 1 | ggcagccaga agcacctgtg ctcccaggat aagggtgagc actcaggatg actgtggaga |
| 61 | aggaggcccc tgatgcgcac ttcactgtgg acaaacagaa catctccctc tggccccgag |
| 121 | agcctcctcc caagtccggt ccatctctgg tcccggggaa aacacccaca gtccgtgctg |
| 181 | cattaatctg cctgacgctg gtcctggtcg cctccgtcct gctgcaggcc gtcctttatc |
| 241 | cccggtttat gggcaccata tcagatgtaa agaccaatgt ccagttgctg aaaggtcgtg |
| 301 | tggacaacat cagcaccctg gattctgaaa ttaaaaagaa tagtgacggc atggaggcag |
| 361 | ctggcgttca gatccagatg gtgaatgaga gcctgggtta tgtgcgttct cagttcctga |
| 421 | agttaaaaac cagtgtggag aaggccaacg cacagatcca gatcttaaca agaagttggg |
| 481 | aagaagtcag taccttaaat gcccaaatcc cagagttaaa aagtgatttg gagaaagcca |
| 541 | gtgctttaaa tacaaagatc cgggcactcc agggcagctt ggagaatatg agcaagttgc |
| 601 | tcaaacgaca aaatgatatt ctacaggtgg tttctcaagg ctggaagtac ttcaagggga |
| 661 | acttctatta cttttctctc attccaaaga cctggtatag tgccgagcag ttctgtgtgt |
| 721 | ccaggaattc acacctgacc tcggtgacct cagagagtga gcaggagttt ctgtataaaa |
| 781 | cagcgggggg actcatctac tggattggcc tgactaaagc agggatggaa ggggactggt |
| 841 | cctgggtgga tgacacgcca ttcaacaagg tccaaagtgt gaggttctgg attccaggtg |
| 901 | agcccaacaa tgctgggaac aatgaacact gtggcaatat aaaggctccc tcacttcagg |
| 961 | cctggaatga tgccccatgt gacaaaacgt ttcttttcat ttgtaagcga ccctatgtcc |
| 1021 | catcagaacc gtgacaggac aggctcccaa gctcactctt tgagctccaa cgcttgttaa |
| 1081 | acatgaggaa atgcctcttt gttccccaga ctccaggatg actttgcacg ttaatttttc |
| 1141 | ttgcttcaaa attgtcccac agtggcattc tggagtccgt ctgtcttggc tggaaattct |
| 1201 | ctgacgtctt ggaggcagct ggaatggaaa ggagaattca ggttaaagtg ggaggggtgg |
| 1261 | gtagagagga tttagaagtt ccaattgccc tgctaaggag gatcaagacc cgtaatccgg |
| 1321 | cataacaccc tggggttttc cactctttca gagaaacctc agcttcatca catcaaagtt |
| 1381 | actccagagc aaccaagcaa ttctcctgat attgtcatcc agggcttttc ttggccaaac |
| 1441 | cccctagaat ttccatgtct ctgcttagct gtgctggcag ctagcagctg gctgtgtttg |
| 1501 | cagtgcaaat agctctgttc ttggaaatcc tgctcatggt atgtccccag tggtttcttc |
| 1561 | atccacatca tctaaagcct gaacccgttc ttctctggtt caagtcagtg gctgacacgg |
| 1621 | acttgtatct ccttcagagc tcggctggca cccagcctcc cttctccttc cactccctta |
| 1681 | gtacactgga gtgccgagcc ctgccttcca cccagcgtcc atccagcccc tgtcctcacc |
| 1741 | tctccggcac ctcctcctcc ttctgcattt cctatcttcc tgtgtcttgt gcatgggaag |
| 1801 | cagccttcag tgccttcatg aattcacctt ccagcttcct cagaataaaa tgctgcctgg |
| 1861 | gtcaaggact caaaaaaaaa aaaaaa |

SEQ ID NO: 30 Human CD207 Amino Acid Sequence (NP_056532.4)

| | |
|---|---|
| 1 | mtvekeapda hftvdkqnis lwprepppks gpslvpgktp tvraaliclt lvlvasvllq |
| 61 | avlyprfmgt isdvktnvql lkgrvdnist ldseikknsd gmeaagvqiq mvneslgyvr |
| 121 | sqflklktsv ekanaqiqil trsweevstl naqipelksd lekasalntk iralqgslen |
| 181 | mskllkrqnd ilqvvsqgwk yfkqnfyyfs lipktwysae qfcvsrnshl tsvtseseqe |
| 241 | flyktaggli ywigltkagm egdwswvddt pfnkvqsvrf wipgepanag nnehcgnika |
| 301 | pslqawndap cdktflfick rpyvpsep |

SEQ ID NO: 31 Mouse CD207 cDNA Sequence (NM_144943.3; CDS: 59-1054)

| | |
|---|---|
| 1 | tttcccgttt ctttctggat aaaaaggtgc ttggggagac agatattgag aatttcctat |
| 61 | gccagaggca gagatgaagg aggaggctcc cgaagcgcac ttcacagtgg acaaacagaa |
| 121 | catctctctc tggcctcgag agcctcctcc caagcaagat ctgtctccag ttctaaggaa |
| 181 | acctctctgt atctgcgtgg ccttcacctg cctggcattg gtgctggtca cctccattgt |
| 241 | gcttcaggct gttttctatc ctaggttgat gggcaaaata ttggatgtga agagtgatgc |
| 301 | ccagatgttg aaaggtcgtg tggacaacat cagcaccctg ggttctgatc ttaagactga |
| 361 | aagaggtcgt gtggacgatg ctgaggttca gatgcagata gtgaacacca ccctcaagag |
| 421 | ggtgcgttct cagatcctgt ctttggaaac cagcatgaag atagccaatg atcagctcca |
| 481 | gatattaaca atgagctggg gagaggttga cagtctcagt gccaaaatcc cagaactgaa |
| 541 | aagagatctg gataaagcca gcgccttgaa cacaaaggtc caaggactac agaacagctt |
| 601 | gccgaatgtc aacaagctgc tcaaacaaca gagtgacatt ctggagatgg tggctcgagg |
| 661 | ctggaagtat ttctcgggga acttctatta cttttcacgc accccaaaga cctggtacag |
| 721 | cgcagagcag ttctgtattt ctagaaaagc tcacctgacc tcagtgtcct cagaatcgga |
| 781 | acaaaagttt ctctacaagg cagcagatgg aattccacac tggattggac ttaccaaagc |
| 841 | agggagcgaa ggggactggt actgggtgga ccagacatca ttcaacaagg agcaaagtag |
| 901 | gaggttctgg attccaggtg aacccaacaa cgcagggaac aatgagcact gtgccaatat |
| 961 | cagggtgact gccctgaagt gctggaacga tggtccctgt gacaatacat ttcttttcat |
| 1021 | ctgcaagagg ccctacgtcc aaacaactga atgacagatc tggcctgagc tcggcatctg |
| 1081 | tggggcaaca gtgacctggc tgaagagatg tctctctccc tgaggctcca agattgctct |
| 1141 | gtactttacg tttttttctt gcttgaaaat tgtcccaaac acagcctgtg gtcttgctgt |
| 1201 | cttggctggc agttctctgc tcctggaggc cttggaggag cttgggttaa acgggtgagg |
| 1261 | acctgaagag ggcgtagcag tccttactgc ccaggcgagg caggtcagca caccaaacag |
| 1321 | gttgtttaga ttttcctgat ccttctcaga agccttggct gaccatataa aagctacatt |
| 1381 | caaatatgac cagtatttga ggaggcagac atgcccaaat ttaaccatga tacaatttat |
| 1441 | acaacatgta ttagaacacc tcatggtatg ctcaaaatat gtaaatatgt tgtttttatg |
| 1501 | tgcctattgc aaataaatgt aatattacta |

SEQ ID NO: 32 Mouse CD207 Amino Acid Sequence (NP_659192.2)

| | |
|---|---|
| 1 | mpeaemkeea peahftvdkq nislwprepp pkqdlspvlr kplcicvaft clalvlvtsi |
| 61 | vlqavfyprl mgkildvdsd aqmlkgrvdn istlgsdlkt ergrvddaev qmgivnttlk |
| 121 | rvrsqilsle tsmkiandql qiltmswgev dslsakipel krdldkasal ntkvqglqns |
| 181 | lenvnkllkq qsdilemvar gwkyfsgnfy yfsrtpktwy saeqfcisrk ahltsvsses |
| 241 | eqkflykaad giphwigltk agsegdwywv dqtsfnkeqs rrfwipgepn nagnnehcan |
| 301 | irvsalkcwn dgpcdntflf ickrpyvqtt e |

SEQ ID NO: 33 Human LRRC25 cDNA Sequence (NM_145256.2; CDS: 643-1560)

| | |
|---|---|
| 1 | gccagaggaa cgccagcgac cccagcagcg ctgcggacgg tgctggccgt ggccgctgcg |
| 61 | gcccccgtgt ccaggtgggc caggacgcag cctctgggcg ccgtcgcttt tccagcatcg |
| 121 | cagaggcaaa agcgtggcag tgggacccaa aaggtaggac tgaggctcta gaacttgcac |
| 181 | ctgtgcaggg actgcaaacc agacctggga ggaccctttc agcagcccdc actccaccct |
| 241 | atcccaggac ttcccagcga cccgccgttc tgggagatac cgggagcgtg atcagggggc |
| 301 | ggggccgttt ccaaggcaac cgcttatttg catagggtcc cgtcctggcc aacgagggcg |
| 361 | ccccaaatgt tcaggacata gaagaagggg ttaactggcc cggatctcct cctcgccttc |
| 421 | caagcccgct aagcactggg gttatctacc cattccccag aaggggagac tgaggcagcc |
| 481 | caccagccaa aggaggcgac cagactgggg ctgcgtttta ccatttcaga agcggcttga |
| 541 | gctggtctga gctataataa taaacactgg cggtggaggc gagggcgacc acagggctga |
| 601 | ggtcagggct aggattccgg tgtctctacg taggttgctt gaatgggggg caccctggca |
| 661 | tggacgctgc tgttgccgct gctgctgcgg gagtcagaca gcctagaacc gtcgtgcacc |
| 721 | gtgtcctccg cggatgtgga ctggaacgcg gagttcagtg ccacgtgcct gaatttcagt |
| 781 | ggcctcagcc tgagcctgcc tcacaaccag tctctgcggg ccagcaacgt gattctcctt |
| 841 | gacctgtctg ggaacggcct gcgagagctt ccagtgacct tctttgccca cctgcagaag |
| 901 | cttgaggtcc tgaacgtgct acgcaacccc ttgtctcgtg tggatggggc gctggccgcc |
| 961 | cgctgtgacc ttgacctgca ggccgactgc aactgtgccc tggagtcctg gcacgacatc |
| 1021 | cgccgagaca actgctctgg ccagaagcct ctgctctgct gggacacaac cagctcccag |
| 1081 | cacaacctct ctgccttcct ggaggtcagc tgcgcccctg gcctggcctc tgcaactatc |
| 1141 | ggggcagtgg tggtcagcgg gtgcctgctt cttggacttg ccatcgctgg ccctgtgctg |
| 1201 | gcctggagac tctggcgatg ccgagtggcc agaagccggg agctgaacaa accctgggct |
| 1261 | gctcaggatg ggcccaagcc cggtttaggc ttgcagccac ggtacggcag ccggagcgcc |
| 1321 | cccaagcccc aagtggccgt gccatcctgc ccctccactc ccgactatga gaacatgttt |
| 1381 | gtgggccagc cagcagccga gcaccagtgg gatgaacaag gggctcaccc ttcagaggac |
| 1441 | aatgactttt acatcaacta caaggacatc gacctggctt cccagcctgt ctactgtaac |
| 1501 | ctgcagtcac tgggccaggc cccaatggat gaagaggagt acgtgatccc cgggcactga |
| 1561 | gcctaacctg tcctaacctc cacccagaac cccttcagtc cctgctgggt gactcagggc |
| 1621 | gtcctaacgc ctccatggcc tcagtttccc catctgaaga atgggtacag gaaaggattg |
| 1681 | tccttgaggc cccaggaagc tctgccgccc cctccctgtc cctcatgccg ctcctcagct |
| 1741 | ccctcagctc ctagaggggg aagaggagag acccccaaca aggggacagg acggtcactg |
| 1801 | tgccaatcct gtcatcaccc tcctgtggat gtacaggcag tgctcaataa atgcttcgag |
| 1861 | gctgatgagg ctgctggctc agggtgcgtg ggttcctcaa ggtggggatt tctgagttct |
| 1921 | aagaccaagt ctccatctga gactcccaaa ttgctcccca cctcccatcc ctgttttttt |
| 1981 | ttgttgttgt tgtttgtttg tttgtttttg aaactgagtg tcactctgtc acccaggctg |
| 2041 | gagtgcaatg ctgcggtctc agctcactgc aacctccgcc tcctgggttc aagtgattct |
| 2101 | cctgcctcag cctcctgagt agctgggatt acagcacccg ccaccatgcc gagctaattt |
| 2161 | ttgtatttat aatagagatg gggtttcgcc atgttggcca ggctggtctc gaactcctga |
| 2221 | cctcaagcga tctgcccgcc tcggcctcct gaagtgctgg gattacaggc gtggccactg |
| 2281 | cgcccaggca cattcctccc ttctgcccct ctcagggccc cttcccaggt ccctgatctc |
| 2341 | caggcttggc ctccagagca gcccacacca accccaaaat aaaaaaatgt atatattcct |
| 2401 | ttaaaaaaaa aaaaaaaaaa aaaaaaaaaa |

SEQ ID NO: 34 Human LRRC25 Amino Acid Sequence (NM_660299.2)

| | |
|---|---|
| 1 | mggtlawtll lplllresds lepsctvssa dvdwnaefsa tclnfsglsl slphnqslra |
| 61 | snvilldlsg nglrelpvtf fahlqklevl nvilnplsrv dgalaarcdl dlqadcncal |
| 121 | eswhdirrdn csgqkpllcw dttssqhnls aflevscapg lasatigavv vsgclllgla |
| 181 | iagpvlawrl wrcrvarsre lnkpwaaqdg pkpglglqpr ygsrsapkpq vavpscpstp |
| 241 | dyenmfvgqp aaehqwdeqg ahpsedndfy inykdidlas qpvycnlqsl gqapmdeeey |
| 301 | vipgh |

SEQ ID NO: 35 Mouse LRRC25 cDNA Sequence (NM_153074.3; CDS: 193-1086)

| | |
|---|---|
| 1 | ctcctcctct cgtgagagcc tgaggctggc agagggctct ctgctgtccc ctccactcct |
| 61 | acacctactc gtcttcccgt cttcccgcag gcgtggatta acaggtggaa agcaccagga |
| 121 | gctgtgaacc ccaacccaga ccctaggacc ctgaggctta cgagacatca cgaaggccag |
| 181 | gaggttgctg ggatgggaag catcagaact aggttgctgt ggttatgtct cctgatgctg |
| 241 | ttggccctgc ttcacaagtc aggaagtcaa gatctcacct gcatggttca cccgagcagg |
| 301 | gtagactgga ctcagacatt taatggcacc tgcctcaatt tcagtggcct tggcctgtcc |
| 361 | ctgccaagga gccccttgca ggccagccat gctcaagtcc tggacctgtc taagaatggc |
| 421 | ctgcaggtgc tccctggggc tttcttcgac aagctggaaa agctgcagac cctgattgtc |
| 481 | acccacaacc agctggacag tgtggacagg tccctggcct tgcgctgtga cctggagctc |
| 541 | aaggcagact gcagctgtgg gctggcctcc tggtatgctc tccgccagaa ctgctccggg |
| 601 | cagcagcagc tactgtgtct acacccagcc accgaagctc caaggaacct ctccaccttc |
| 661 | cttcaggtca gctgtccccc cagctggggc ccggggacca ttggagccct tgttgctggg |
| 721 | actatctccc tggctgtggc tgtcagtgga tctgtgctgg cctggagact tcttcgccgc |
| 781 | cgccgcagag ccagtgagca cagcctcagc aaagcccaga tgtccccaca cgatatcccc |
| 841 | aaaccagtga cagatttcct gccaaggtac agcagccggc gacctggccc caaggcccca |
| 901 | gactcaccac ccagcaggtt cacaatggat tatgagaatg tctttattgg ccaggrggcc |
| 961 | gaggactgct catggtctgc agccagaaac agcccttctg gggacagtga ctgctacatg |
| 1021 | aactacagga gtgtcgacca ggactctcag cccgtctatt gcaacctgga gtccctgggg |
| 1081 | cgatgaggag agtgtggtct cctggcgctg aaccagcctc cgacagcccc caggatccag |
| 1141 | cacgctcaac atcacagggg gtagaggaca ccccaccccc ccacccccaa agcagaagga |
| 1201 | gggtcagaaa caaccctccg gtcagtgtgc atgcatgtga tgctcaataa aagctctggg |
| 1261 | agcagctgac tct |

SEQ ID NO: 36 Mouse LRRC25 Amino Acid Sequence (NP_694714.1)

| | |
|---|---|
| 1 | mgsirtrllw lcllmllall hksgsqdltc mvhpsrvdwt qtfngtclnf sglglslprs |
| 61 | plqashaqvl dlsknglqvl pgaffdklek lqtlivthnq ldsvdrslal rcdlelkadc |
| 121 | scglaswyal rqncsgqqql lclhpateap rnlstflqvs cppswgpgti galvagtisl |
| 181 | avavsgsvla wrllrrrrra sehslskaqm sphdipkpvt dflpryssrr pgpkapdspp |
| 241 | srftmdyenv figqpaedcs wsaarnspsg dsdcymnyrs vdqdsqpvyc nleslgr |

SEQ ID NO: 37 Human SELPLG Transcript Variant 1 cDNA Sequence
(NM_001206609.1; CDS: 178-1464)

| | |
|---|---|
| 1 | aatcatccga gaaccttgga gggtggacag tgcccctttt acagatgaga aaactgaggc |
| 61 | ttgaagggga gaagcagctg cctctggcgg catggcttct ggctgcagga tgcccatgga |
| 121 | gttcgtggtg accctaggcc tgtgtctcgg cttcctttgc tgaacttgaa caggaagatg |
| 181 | gcagtggggg ccagtggtct agaaggagat aagatggctg gtgccatgcc tctgcaactc |
| 241 | ctcctgttgc tgatcctact gggccctggc aacagcttgc agctgtggga cacctgggca |
| 301 | gatgaagccg agaaagcctt gggtcccctg cttgcccggg accggagaca ggccaccgaa |
| 361 | tatgagtacc tagattatga tttcgtgcca gaaacggagc ctccagaaat gctgaggaac |
| 421 | agcactgaca ccactcctct gactgggcct ggaacccctg agtctaccac tgtggagcct |
| 481 | gctgcaaggc gttctactgg cctggatgca ggaggggcag tcacagagct gaccacggag |
| 541 | ctggccaaca tggggaacct gtccacggat tcagcagcta tggagataca gaccactcaa |
| 601 | ccagcagcca cggaggcaca gaccactcaa ccagtgccca cggaggcaca gaccactcca |
| 661 | ctggcagcca cagaggcaca gacaactcga ctgacggcca cggaggcaca gaccactcca |
| 721 | ctggcagcca cagaggcaca gaccactcca ccagcagcca cggaagcaca gaccactcaa |
| 781 | cccacaggcc tggaggcaca gaccactgca ccagcagcca tggaggcaca gaccactgca |
| 841 | ccagcagcca tggaagcaca gaccactcca ccagcagcca tggaggcaca gaccactcaa |
| 901 | accacagcca tggaggcaca gaccactgca ccagaagcca cggaggcaca gaccactcaa |
| 961 | cccacagcca cggaggcaca gaccactcca ctggcagcca tggaggccct gtccacagaa |
| 1021 | cccagtgcca cagaggccct gtccatggaa cctactacca aaagaggtct gttcataccc |
| 1081 | ttttctgtgt cctctgttac tcacaagggc attcccatgg cagccagcaa tttgtccgtc |
| 1141 | aactacccag tgggggcccc agaccacatc tctgtgaagc agtgcctgct ggccatccta |
| 1201 | atcttggcgc tggtggccac tatcttcttc gtgtgcactg tggtgctggc ggtccgcctc |
| 1261 | tcccgcaagg gccacatgta ccccgtgcgt aattactccc ccaccgagat ggtctgcatc |
| 1321 | tcatccctgt tgcctgatgg gggtgagggg ccctctgcca cagccaatgg gggcctgtcc |
| 1381 | aaggccaaga gcccgggcct gacgccagag cccagggagg accgtgaggg ggatgacctc |
| 1441 | accctgcaca gcttcctccc ttagctcact ctgccatctg ttttggcaag accccacctc |
| 1501 | cacgggctct cctgggccac ccctgagtgc ccagacccca ttccacagct ctgggcttcc |
| 1561 | tcggagaccc ctggggatgg ggatcttcag ggaaggaact ctggccaccc aaacaggaca |
| 1621 | agagcagcct ggggccaagc agacgggcaa gtggagccac ctctttcctc cctccgcgga |
| 1681 | tgaagcccag ccacatttca gccgaggtcc aaggcaggag gccatttact tgagacagat |
| 1741 | tctctccttt ttcctgtccc ccatcttctc tgggtccctc taacatctcc catggctctc |
| 1801 | cccgcttctc ctggtcactg gagtctcctc cccatgtacc caaggaagat ggagctcccc |
| 1861 | catcccacac gcactgcact gccattgtct tttggttgcc atggtcacca aacaggaagt |
| 1921 | ggacattcta agggaggagt actgaagagt gacggacttc tgaggctgtt tcctgctgct |
| 1981 | cctctgactt ggggcagctt gggtcttctt gggcacctct ctgggaaaac ccagggtgag |
| 2041 | gttcagcctg tgagggctgg gatgggtttc gtgggcccaa gggcagacct ttctttggga |
| 2101 | ctgtgtggac caaggagctt ccatctagtg acaagtgacc cccagctatc gcctcttgcc |
| 2161 | ttcccctgtg gccactttcc agggtggact ctgtcttgtt cactgcagta tcccaactgc |
| 2221 | aggtccagtg caggcaataa atatgtgatg gacaaacgat aggggaatcc ttcaaggttt |
| 2281 | caaggctgtc tccttcaggc agccttcccg gaattctcca tccctcagtg caggatgggg |
| 2341 | gctggtcctc agctgtctgc cctcagcccc tggcccccca ggaagcctct ttcatgggct |
| 2401 | gttaggttga cttcagtttt gcctcttgga caacaggggg tcttgtacat ccttgggtga |
| 2461 | ccaggaaaag ttcaggctat ggggggccaa agggagggct gccccttccc caccagtgac |
| 2521 | cactttattc cacttcctcc attacccagt tttggcccac agagtttggt cccccccaaa |
| 2581 | cctcggacca atatccctct aaacatcaat ctatcctcct gttaaagaaa aaaaaaaa |

SEQ ID NO: 38 Human SELPLG Isform 1 Amino Acid Sequence (NP_001193538.1)

| | |
|---|---|
| 1 | mavgasgleg dkmagamplq lllllillgp gnslqlwdtw adeaekalgp llardrrqat |
| 61 | eyeyldydfl peteppemlr nstdttpltg pgtpesttve paarrstgld aggavteltt |
| 121 | elanmgnlst dsaameiqtt qpaateaqtt qpvpteaqtt plaateaqtt rltateaqtt |
| 181 | plaateaqtt ppaateaqtt qptgleaqtt apaameaqtt apaameaqtt ppaameaqtt |
| 241 | qttameaqtt apeateaqtt qptateaqtt plaamealst epsatealsm epttkrglfi |
| 301 | pfsvssvthk gipmaasnls vnypvgapdh isvkqcllai lilalvatif fvctvvlavr |
| 361 | lsrkghmypv rnysptemvc issllpdgge gpsatanggl skakspgltp epredregdd |
| 421 | ltlhsflp |

SEQ ID NO: 39 Human SELPLG Transcript Variant 2 cDNA Sequence
(NM_003006.4; CDS: 161-1399)

| | |
|---|---|
| 1 | acacacagcc attgggggtt gctcggatcc gggactgccg cagggggtgc cacagcagtg |
| 61 | cctggcagcg tgggctggga ccttgtcact aaagcagaga agccacttct tctgggccca |
| 121 | cgaggcagct gtcccatgct ctgctgagca cggtggtgcc atgcctctgc aactcctcct |
| 181 | gttgctgatc ctactgggcc ctggcaacag cttgcagctg tgggacacct gggcagatga |
| 241 | agccgagaaa gccttgggtc ccctgcttgc ccgggaccgg agacaggcca ccgaatatga |
| 301 | gtacctagat tatgatttcc tgccagaaac ggagcctcca gaaatgctga ggaacagcac |
| 361 | tgacaccact cctctgactg ggcctggaac ccctgagtct accactgtgg agcctgctgc |
| 421 | aaggcgttct actggcctgg atgcaggagg ggcagtcaca gagctgacca cggagctggc |
| 481 | caacatgggg aacctgtcca cggattcagc agctatggag atacagacca ctcaaccagc |
| 541 | agccacggag gcacagacca ctcaaccagt gcccacggag gcacagacca ctccactggc |
| 601 | agccacagag gcacagacaa ctcgactgac ggccacggag gcacagacca ctccactggc |
| 661 | agccacagag gcacagacca ctccaccagc agccacggaa gcacagacca ctcaacccac |
| 721 | aggcctggag gcacagacca ctgcaccagc agccatggag gcacagacca ctgcaccagc |
| 781 | agccatggaa gcacagacca ctccaccagc agccatggag gcacagacca ctcaaaccac |
| 841 | agccatggag gcacagacca ctgcaccaga agccacggag gcacagacca ctcaacccac |
| 901 | agccacggag gcacagacca ctccactggc agccatggag gccctgtcca cagaacccag |
| 961 | tgccacagag gccctgtcca tggaacctac taccaaaaga ggtctgttca tacccttttc |
| 1021 | tgtgtcctct gttactcaca agggcattcc catggcagcc agcaatttgt ccgtcaacta |
| 1081 | cccagtgggg gccccagacc acatctctgt gaagcagtgc ctgctggcca tcctaatctt |
| 1141 | ggcgctggtg gccactatct tcttcgtgtg cactgtggtg ctggcggtcc gcctchcccg |
| 1201 | caagggccac atgtaccccg tgcgtaatta ctcccccacc gagctggtct gcatctcatc |
| 1261 | cctgttgcct gatgggggtg aggggccctc tgccacagcc aatgggggcc tgtccaaggc |
| 1321 | caagagcccg ggcctgacgc cagagcccag ggaggaccgt gagggggatg acctcaccct |
| 1381 | gcacagcttc ctcccttagc tcactctgcc atctgttttg gcaagacccc acctccacgg |
| 1441 | gctctcctgg gccacccctg agtgcccaga ccccattcca cagctctggg cttcctcgga |
| 1501 | gacccctggg gatggggatc ttcagggaag gaactctggc cacccaaaca ggacaagagc |
| 1561 | agcctggggc caagcagacg ggcaagtgga gccacctctt tcctccctcc gcggatgaag |
| 1621 | cccagccaca tttcagccga ggtccaaggc aggaggccat ttacttgaga cagattctct |
| 1681 | cctttttcct gtcccccatc ttctctgggt ccctctaaca tctcccatgg ctctccccgc |
| 1741 | ttctcctggt cactggagtc tcctccccat gtacccaagg aagatggagc tcccccatcc |
| 1801 | cacacgcact gcactgccat tgtcttttgg ttgccatggt caccaaacag gcagtggaca |
| 1861 | ttctaaggga ggagtactga agagtgacgg acttctgagg ctgtttcctg ctgctcctct |
| 1921 | gacttggggc agcttgggtc ttcttgggca cctctctggg aaaacccagg gtgaggttca |
| 1981 | gcctgtgagg gctgggatgg gtttcgtggg cccaagggca gacctttctt tgggactgtg |
| 2041 | tggaccaagg agcttccatc tagtgacaag tgacccccag ctatcgcctc ttgccttccc |
| 2101 | ctgtggccac tttccagggt ggactctgtc ttgttcactg cagtatccca actgcaggtc |
| 2161 | cagtgcaggc aataaatatg tgatggacaa acgatagcgg aatccttcaa ggtttcaagg |
| 2221 | ctgtctcctt caggcagcct tcccggaatt ctccatccct cagtgcagga tgggggctgg |
| 2281 | tcctcagctg tctgccctca gcccctggcc ccccaggaag cctctttcat gggctgttag |
| 2341 | gttgacttca gttttgcctc ttggacaaca gggggtcttg tacatccttg ggtgaccagg |
| 2401 | aaaagttcag gctatggggg gccaaaggga gggctgcccc ttccccacca gtgaccactt |
| 2461 | tattccactt cctccattac ccagttttgg cccacagagt ttggtccccc ccaaacctcg |
| 2521 | gaccaatatc cctctaaaca tcaatctatc ctcctgttaa agaaaaaaaa aaa |

SEQ ID NO: 40 Human SELPLG Isoform 1 Amino Acid Sequence (NP_002997.2)

| | |
|---|---|
| 1 | mplqllllll ilgpgnslql wdtwadeaek algpllardr rqateyeyld ydflpetepp |
| 61 | emlrnstdtt pltgpgtpes ttvepaarrs tgldaggdvt elttelanmg nlstdsaame |
| 121 | iqttqpaate aqttgpvpte agttplaate aqttrltate aqttplaate aqttppaate |
| 181 | aqttqptgle aqttapaame aqttapaame aqttppaame aqttgttame aqttapeate |
| 241 | aqttqptate aqttplaame alstepsate alsmepttkr glfipfsvss vthkgipmaa |
| 301 | snlsvnypvg apdhisvkqc llaililalv atiffvctvv lavrlsrkgh mypvrnyspt |
| 361 | emvcissllp dggegpsata ngglskaksp gltpepredr egddltlhsf lp |

SEQ ID NO: 41 Mouse SELPLG cDNA Sequence (NM_009151.3; CDS: 159-1412)

| | |
|---|---|
| 1 | attctcgctt ccttcttcca caccctgccg ttgggggttg gcgggcagat tgggaccaca |
| 61 | agtgtctggc agtgtggact ggggccctgt cactgaggca gagtcgtttg cttctgggcc |
| 121 | ctgaggcagc tgccccatgc tctgttgggc acggtaccat gtccccaagc ttccttgtgc |
| 181 | tgctgaccat cttgggccct ggcaacagcc ttcagctgca ggacccctgg gggcatgaaa |
| 241 | ccaaggaagc cccgggtcct gtgcatctcc gggaacggag gcaggtggtt ggggatgacg |
| 301 | attttgagga ccctgactat acgtataaca cagacccccc agaattgctg aaaaatgtca |
| 361 | ccaacaccgt ggctgctcac cctgagctgc caaccaccgt ggtcatgcta gagagagatt |
| 421 | ccacgagcgc tggaacctcc gagagagcca ctgagaagat tgccaccact gaccctactg |
| 481 | ccccaggtac aggagggaca gctgttggga tgctgagcac agactctgcc acacagtgga |
| 541 | gtctaacctc agtagagacc gtccaaccag catccacaga ggtagagacc tcgcagccag |
| 601 | cacccatgga ggcagagacc tcgcagccag cacccatgga ggcagagacc tcgcagccag |
| 661 | cacccatgga ggcagagacc tcgcagccag cacccatgga ggcagacacc tcgcagccag |
| 721 | cacccatgga ggcagacacc tcaaagccag cacccacgga ggcagagacc tcaaagccag |
| 781 | cacccacgga ggcagagacc tctcagccag cacccaacga ggcagagacc tcaaaaccag |
| 841 | cacccacgga ggcagagacc tcaaaaccag cacccacgga ggcagagacc acccagcttc |
| 901 | ccaggattca ggctgtaaaa actctgttta caacgtctgc agccaccgaa gtcccttcca |
| 961 | cagaacctac caccatggag acggcgtcca cagagtctaa cgagtctacc atcttccttg |
| 1021 | ggccatccgt gactcactta cctgacagcg gcctgaagaa agggctgatt gtgacccctg |
| 1081 | ggaattcacc tgccccaacc ctgccaggga gttcagatct catcccggtg aagcaatgtc |
| 1141 | tgctgattat cctcatcttg gcttctctgg ccaccatctt cctcgtgtgc acagtggtgc |
| 1201 | tggcggtccg tctgtcccgt aagacccaca tgtacccagt gcggaactac tcccccacgg |
| 1261 | agatgatctg catctcgtcc ctgctacctg aggggggaga cggggcccct gtcacagcca |
| 1321 | atgggggcct gcccaaggtc caggacctga agacagagcc cagtggggac cgggatgggg |
| 1381 | acgacctcac cctgcacagc ttcctccctt agactcccct gcctgcccac ctaagcgaga |
| 1441 | catttgctag ctccactctc acccgctggt cacagaggtc atagatctgg gcttcctggg |
| 1501 | tgaaatgtat tcacgggagt ctttagagcg cccaccgctg tgtgtctccc tgcaggtcac |
| 1561 | tggatacctg tccttgcgtt ctccagaaag actcagctcc cttattccac tcccaaaagc |
| 1621 | tactctgttg gttgccatgg taacccggta agagaggagc tttgtgggag gccgccatgt |
| 1681 | ctgcttctct gattccagtg gcaggtagcc tggctttccc aggtccctgg cttggaggga |
| 1741 | tggtccttcc tttgggcccg tgtgaaccaa cgagtttccg tacagtgaca gaatgacctc |
| 1801 | gcgctgcggc ctggcccagc acaggcatcc aataaacata ttataataaa cgatagctga |
| 1861 | gtccttcatg tgcctaggct gccatctcca gccctcccgg agggcgctta gaccattgtc |
| 1921 | cacaccgctc ttagacatct aatacatgct tgggcaactg caaggggcac tggagggttt |
| 1981 | aagcgacacg tggtaggtag catatgccca tcacccaagc agtacaggag ttcaaggtca |
| 2041 | tccttggctc gttaactgcc tgggctacat gagaccctgt ctccgagaaa actaaagctg |
| 2101 | ggtctggctg gctggctcag ccggtgaagg tgcttgctgc tacgcctcat ggcctaagct |
| 2161 | ccaggggggc cctcatggtg gaaggagaag gctgactctc caaaactgtt ctctggcatc |
| 2221 | catactcaca ggtaaatatg aacacaacta cacaagctag agaactttat tgaatctacc |
| 2281 | ctttccaagg tgggtcaaag gaggaaggtc cctttggtgt tggccaattt ctttttaaag |
| 2341 | atttatttat gtttatgagt atgctgtcac tgtcttcaga cacagcagaa gagggcatcg |
| 2401 | gatcccatta cagacttgag ccaccccgtg ggtactggga attgaactca ggaactctgg |
| 2461 | aagagcagtc agtgctctta acagctgagc catcccttca gcagccctgt cagatttttt |
| 2521 | tttttaatca ccaaagagat tttattcaag tttctaacac aaatgagtta ttttggtttt |
| 2581 | gaattacaga actaagtcca aact |

SEQ ID NO: 42 Mouse SELPLG Amino Acid Sequence (NP_033177.3)

| | |
|---|---|
| 1 | mspsflvllt ilgpgnslql qdpwghetke apgpvhlrer rqvvgdddfe dpdytyntdp |
| 61 | pellknvtnt vaahpelptt vvmlerdsts agtseratek iattdptapg tggtavgmls |
| 121 | tdsatqwslt svetvqpast evetsqpapm eaetsqpapm eaetsqpapm eaetsqpapm |
| 181 | eadtsqpapm eadtskpapt eaetskpapt eaetsqpapn eaetskpapt eaetskpapt |
| 241 | eaettqlpri qavktlftts aatevpstep ttmetastes nestiflgps vthlpdsglk |
| 301 | kglivtpgns paptlpgssd lipvkqclli ililaslati flvctvvlav rlsrkthmyp |
| 361 | vrnysptemi cissllpegg dgapvtangg lpkvqdlkte psgdrdgddl tlhsflp |

SEQ ID NO: 43 Human AIF1 Transcript Variant 1 cDNA Sequence (NM_032955.2;
CDS: 113-394)

| | |
|---|---|
| 1 | cgttgtctcc tccacctagc agttggttgg caaccccttc ctcagtcccc tgctgaaaac |
| 61 | cctccagtca gcgcttatcc cttctgctct ctcccctcac ccagagaaat acatggagtt |
| 121 | tgaccttaat ggaaatggcg atattgatat catgtccctg aaacgaatgc tggagaaact |
| 181 | tggagtcccc aagactcacc tagagctaaa gaaattaatt ggagaggtgt ccagtggctc |
| 241 | cggggagacg ttcagctacc ctgactttct caggatgatg ctgggcaaga gatctgccat |
| 301 | cctaaaaatg atcctgatgt atgaggaaaa agcgagagaa aaggaaaagc caacaggccc |
| 361 | cccagccaag aaagctatct ctgagttgcc ctgatttgaa gggaaaaggg atgatgggat |
| 421 | tgaaggggct tctaatgacc cagatatgga aacagaagac aaaattgtaa gccagagtca |
| 481 | acaaattaaa taaattaccc cctcctccag atcaagtca |

SEQ ID NO: 44 Human AIF1 Transcript Variant 4 cDNA Sequence
(NM_001318970.1; CDS: 223-504)

| | |
|---|---|
| 1 | aggggaaagg ggaagtttgg gaggaaggct tgtgagaaga ctggtgggag agaaggagag |
| 61 | cctgcagaca gaggcctcca gcttggagga aaagctttcg gactgctgaa ggccgagcag |
| 121 | gaagagaggc tggatgagat caacaagcaa ttcctagacg atcccaaata tagcagtgat |
| 181 | gaggatctgc cctccaaact ggaaggcttc aaagagaaat acatggagtt tgaccttaat |
| 241 | ggaaatggcg atattgatat catgtccctg aaacgaatgc tggagaaact tggagtcccc |
| 301 | aagactcacc tagagctaaa gaaattaatt ggagaggtgt ccagtggctc cggggagacg |
| 361 | ttcagctacc ctgactttct gaggatgatg ctgggcaaga gatctgccat cctaaaaatg |
| 421 | atcctgatgt atgaggaaaa agcgagagaa aaggaaaagc caacaggccc cccagccaag |
| 481 | aaagctatct ctgagttgcc ctgatttgaa gggaaaaggg atgatgggat tgaaggggct |
| 541 | tctaatgacc cagatatgga aacagaagac aaaattgtaa gccagagtca acaaattaaa |
| 601 | taaattaccc cctcctccag atcaagtca |

SEQ ID NO: 45 Human AIF1 Isoform 1 Amino Acid Sequence (NP_001305899.1
and NP_116573.1)

| | |
|---|---|
| 1 | mefdlngngd idimslkrml eklgvpkthl elkkligevs sgsgetfsyp dflrmmlgkr |
| 61 | sailkmilmy eekarekekp tgppakkais elp |

SEQ ID NO: 46 Human AIF1 Transcript Variant 3 Sequence (NM_001623.4; CDS:
122-565)

| | |
|---|---|
| 1 | aggggaaagg ggaagtttgg gaggaaggct tctgagaaga ctggtgggag agaaggagag |
| 61 | cctgcagaca gaggcctcca gcttggtctg tctccccacc tctaccagca tctgctgagc |
| 121 | tatgagccaa accagggatt tacagggagg aaaagctttc ggactgctga aggcccagca |
| 181 | ggaagagagg ctggatgaga tcaacaagca attcctagac gatcccaaat atagcagtga |
| 241 | tgaggatctg ccctccaaac tggaaggctt caaagagaaa tacatggagt ttgaccttaa |
| 301 | tggaaatggc gatattgata tcatgtctgt gaaacgaatg ctggagaaac ttggagtccc |
| 361 | caagactcac ctagagctaa agaaattaat tggagaggtg tccagtggct ccggggagac |
| 421 | gttcagctac cctgactttc tcaggatgat gctgggcaag agatctgcca tcctaaaaat |
| 481 | gatcctgatg tatgaggaaa aagcgagaga aaaggaaaag ccaacaggcc ccccagccaa |
| 541 | gaaagctatc tctgagttgc cctgatttga agggaaaagg gatgatggga ttgaaggggc |
| 601 | ttctaatgac ccagatatgg aaacagaaga caaaattgta agccagagtc aacaaattaa |
| 661 | ataaattacc ccctcctcca gatcaagtca |

SEQ ID NO: 47 Human AIF1 Isoform 3 Amino Acid Sequence (NP_001614.3)

| | |
|---|---|
| 1 | msqtrdlqgg kafgllkaqq eerldeinkq flddpkyssd edlpsklegf kekymefdln |
| 61 | gngdidimsl krmleklgvp kthlelkkli gevssgsget fsypdflrmm lgkrsailkm |
| 121 | ilmyeekare kekptgppak kaiselp |

SEQ ID NO: 48 Mouse AIF1 Transcript Variant 1 cDNA Sequence
(NM_001361501.1; CDS: 369-812)

| | |
|---|---|
| 1 | atcctattgt ttccctgtca ggctcctcca aggcccaaac taactggagc cagacgaacc |
| 61 | ctctgatgtg gtctgcacag ggcgctaggc tcagctcacc ccattcctgg agcagcctgc |
| 121 | agacttcatc ctctctcttc catcccgggg aaagtcagcc agtcctcctc agctgcctgt |
| 181 | cttaacctgc atcatgaagc ctgaggagat ttcaacagaa gctgatgtgg aagtgatgcc |
| 241 | tgggagttag caagggaatg agtggaaagg ggaagtgtga gaacggtccc agaagagact |
| 301 | ggggagctgg tggagagagg acccagcgga cagactgcca gcctaagaca accagcgtct |
| 361 | gaggagccat gagccaaagc agggatttgc agggaggaaa agcttttgga ctgctgaagg |
| 421 | cccagcagga agagaggctg gaggggatca acaagcaatt cctcgatgat cccaaataca |
| 481 | gcaatgatga ggatctgccg tccaaacttg aagccttcaa ggtgaagtac atggagtttg |
| 541 | atctgaatgg aaatggagat atcgatatta tgtccttgaa gcgaatgptg gagaaacttg |
| 601 | gggttcccaa gacccdccta gagctgaaga gattaattag agaggtgtcc agtggptccg |
| 661 | aggagacgtt cagctactct gactttctca gaatgatgct gggcaagaga tctgccatct |
| 721 | tgagaatgat tctgatgtat gaggagaaaa acaaagaaca caagaggcca actggtcccc |
| 781 | cagccaagaa agctatctcc gagctgccct gattggaggt ggatgtcaca cggtggggct |
| 841 | gagtgaggag cttctgatga cagcagcatg gaaaaaagaa acagtcgtga gpcagagtca |
| 901 | gactaaataa atgacgctcc tagtgggtca catca |

SEQ ID NO: 49 Mouse AIF1 Transcript Variant 2 cDNA Sequence (NM_019467.3;
CDS: 366-809)

| | |
|---|---|
| 1 | atcctattgt ttccctgtca ggctcctcca aggcccaaac taactggagc cagacgaacc |
| 61 | ctctgatgtg gtctgcacag ggcgctaggc tcagctcacc ccattcctgg agcagcctgc |
| 121 | agacttcatc ctctctcttc catcccgggg aaagtcagcc agtcctcctc agctgcctgt |
| 181 | cttaacctgc atcatgaagc ctgaggagat ttcaaaagct gatgtggaag tgatgcctgg |
| 241 | gagttagcaa gggaatgagt ggaaagggga agtgtgagaa cggtcccaga agagactggg |
| 301 | gagctggtgg agagaggacc cagcggacag actgccagcc taagacaacc agcgtctgag |
| 361 | gagccatgag ccaaagcagg gatttgcagg gaggaaaagc ttttggactg ctgaaggccc |
| 421 | agcaggaaga gaggctggag gggatcaaca agcaattcct cgatgatccc aaatacagca |
| 481 | atgatgagga tctgccgtcc aaacttgaag ccttcaaggt gaagtacatg gagtttgatc |
| 541 | tgaatggaaa tggagatatc gatattatgt ccttgaagcg aatgctggag aaacttgggg |
| 601 | ttcccaagac ccacctagag ctgaagagat taattagaga ggtgtccagt ggctccgagg |
| 661 | agacgttcag ctactctgac tttctcagaa tgatgctggg caagagatct gccatcttga |
| 721 | gaatgattct gatgtatgag gagaaaaaca aagaacacaa gaggccaact ggtcccccag |
| 781 | ccaagaaagc tatctccgag ctgccctgat tggaggtgga tgtcacacgg tggggctgag |
| 841 | tgaggagctt ctgatgacag cagcatggaa aaaagaaaca gtcgtgagcc agagtcagac |
| 901 | taaataaatg acgctcctag tgggtcacat caaaaaaaaa aaaaaaaa |

SEQ ID NO: 50 Mouse AIF1 Isoform A Amino Acid Sequence (NP_001348430.1
and NP_062340.1)

| | |
|---|---|
| 1 | msqsrdlqgg kafgllkaqq eerleginkq flddpkysnd edlpskleaf kvkymefdln |
| 61 | gngdidimsl krmleklgvp kthlelkrli revssgseet fsysdflrmm lgkrsaiilm |
| 121 | ilmyeeknke hkrptgppak kaiselp |

SEQ ID NO: 51 Mouse AIF1 Transcript Variant 3 cDNA Sequence
(NM_001361502.1; CDS: 194-634)

| | |
|---|---|
| 1 | atcctattgt ttccctgtca ggctcctcca aggcccaaac taactggagc cagacgaacc |
| 61 | ctctgatgtg gtctgcacag ggcgctaggc tcagctcacc ccattcctgg agcagcctgc |
| 121 | agacttcatc ctctctcttc catcpcgggg aaagtcagcc agtcptcctc agctgcctgt |
| 181 | cttaacctgc atcatgaagc ctgaggagat ttcaagagga aaagcttttg gactgctgaa |
| 241 | ggcccagcag gaagagaggc tggaggggat caacaagcaa ttcctcgatg atcccaaata |
| 301 | cagcaatgat gaggatctgc cgtccaaact tgaagccttc aaggtgaagt acatggagtt |
| 361 | tgatctgaat ggaaatggag atatcgatat tatgtccttg aagcgaatgc tggagaaact |
| 421 | tggggttccc aagacccacc tagagctgaa gagattaatt agagaggtgt ccagtggctc |
| 481 | cgaggagacg ttcagctact ctgactttct cagaatgatg ctgggcaaga gatctgccat |
| 541 | cttgagaatg attctgatgt atgaggagaa aaacaaagaa cacaagaggc caactggtcc |
| 601 | cccagccaag aaagctatct ccgagctgcc ctgattggag gtggatgtca cacggtgggg |
| 661 | ctgagtgagg agcttctgat gacagcagca tggaaaaaag aaacagtcgt gagccagagt |
| 721 | cagactaaat aaatgacgct cctagtgggt cacatca |

SEQ ID NO: 52 Mouse AIF1 Isoform B Amino Acid Sequence (NP_001348431.1)

| | |
|---|---|
| 1 | mkpeeisrgk afgllkaqqe erleginkqf lddpkysnde dlpskleafk vkymefdlng |
| 61 | ngdidimslk rmleklgvpk thlelkrlir evssgseetf sysdflrmml gkrsailrmi |
| 121 | lmyeeknkeh krptgppakk aiselp |

SEQ ID NO: 53 Human CD84 Transcript Variant 1 cDNA Sequence
(NM_001184879.1; CDS: 80-1117)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctggccg gaagcagctg gaaaagactc agaaatcttc acagtgaatg ggattctggg |
| 181 | agagtcagtc actttccctg taaatatcca agaaccacgg caagttaaaa tcattgcttg |
| 241 | gacttctaaa acatctgttg cttatgtaac accaggagac tcagaaacag cacccgtagt |
| 301 | tactgtgacc cacagaaatt attatgaacg gatacatgcc ttaggtccga actacaatct |
| 361 | ggtcattagc gatctgagga tggaagacgc aggagactac aaagcagaca taaatacaca |
| 421 | ggctgatccc tacaccacca ccaagcgcta caacctgcaa atctatcgtc ggcttgggaa |
| 481 | accaaaaatt acacagagtt taatggcatc tgtgaacagc acctgtaatg tcacactgac |
| 541 | atgctctgta gagaaagaag aaaagaatgt gacatacaat tggagtcccc tgggagaaga |
| 601 | gggtaatgtc cttcaaatct tccagactcc tgaggaccaa gagctgactt acacgtgtac |
| 661 | agcccagaac cctgtcagca acaattctga ctccatctct gcccggcagc tctgtgcaga |
| 721 | catcgcaatg ggcttccgta ctcaccacac cgggttgctg agcgtgctgg ctatgttctt |
| 781 | tctgcttgtt ctcattctgt cttcagtgtt tttgttccgt ttgttcaaga gaagacaagg |
| 841 | taggattttc ccagaaggtt cctgcttgaa caccttcact aagaaccctt atgctgcctc |
| 901 | aaagaaaacc atatacacat atatcatggc ttcaaggaac acccagccag cagagtccag |
| 961 | aatctatgat gaaatcctgc agtccaaggt gcttccctcc aaggaagagc cagtgaacac |
| 1021 | agtttattcc gaagtgcagt ttgctgataa gatggggaaa gccagcacac aggacagtaa |
| 1081 | acctcctggg acttcaagct atgaaattgt gatctaggct gctgggctga attctccctc |
| 1141 | tggaaactga gttacaacca ccaatactgg caggttccct ggatccagat cttctctgcc |
| 1201 | caactcttac tgggagattg caaactgcca catctcagcc tgtaagcaaa gcaggaaacc |
| 1261 | ttctgctggg catagcttgt gcctaaatgg acaaatggat gcataccctt cctgaaatga |
| 1321 | ctcccttctg aatgaatgac aaagcaggtt acctagtata gttttcccaa acttcttccc |
| 1381 | atcatagcac atgtagaaaa taatattttt atggcacact gggataaaca agcaagattg |
| 1441 | ctcacttctg gaagctgcat atgactagag gcctcttgtg actggaggta acaaccctgc |
| 1501 | ccagtaactg tgggagaagg ggatcaatat tttgcacacc tgtaataggc catggcacac |
| 1561 | cagccaagat gctctgctca cagtcagtat gtgtgaagat ccctggtgcg tggccttcac |
| 1621 | cacgcatctt gagcaaatta ggaaaatgta cccttcgctt gaggcagatg cagcccttcc |
| 1681 | cccgagtgca tggcttggag agcagaatgt gggctgcata taagcacact catccctttg |
| 1741 | tctgggaatc tttgtgcagg gcataacagg cttagtaagt ccaaacacag atgacagtgc |
| 1801 | tgtagtgggt tctgtcagag ttgtggctct cagccatgta gacacactct ccaaatggag |
| 1861 | tgttggaaaa tgttctttct gcagggtcta gagactgctg ggacactttt cttggagtgc |
| 1921 | tacttcagaa gccttatagg attttctttc tggccaagat ttccttctgt atcactccaa |
| 1981 | gcagcctcag cagaagaagc agccatgccc agtattccca ctctccaaaa ggaactgacc |
| 2041 | agcttatatt tctcacactt ctggggaact gggtataatc caaccatcaa aatagaagac |
| 2101 | cttgcaagaa gcagagtcat tctccagaag gaacttggga gatgatggtg cagatgatga |
| 2161 | aactgggttc atcccagttc caaagactca gagaactaga gtttaagctg aggcagagtg |
| 2221 | ccgccaccct ggcatgcccc acaaacagat caccagccag cttacacagg cattaactct |
| 2281 | cctcaatgag gaagaatcat tcacaactga gcaagacatt catatgatca tttaaggaag |
| 2341 | tgtttccctt atgtgttagc aagtataatc ggctaactcc taaatcccaa tgaatagtcc |
| 2401 | taggctggac agcaatgggc tgcaattagg cagataaaga catcagtccc agtaaatgaa |
| 2461 | tccatagact catctagcac caactaccat tagcactatg ttaggagctg caaggcccca |
| 2521 | aagtagaaga tgtgcataat gtctgctctt gtgtagctca ggagacaatt caaggcccca |
| 2581 | cactacagtt aacgctgaac tgcagctgca agtaatagca tgaacagtca gaaaaatacc |
| 2641 | ttatgagggg gcagggctga agctgggcct tgaaggatgg atgaaatttg gatagagaat |
| 2701 | gaggaagaca gagggcctcc aagtgagaga agcatgaaaa atgagcaggg gcctggatca |
| 2761 | gtggggtgta ttcagagcac ctctccagat gcaccatgca tgctcacagt cccttgccta |
| 2821 | tgtgtggcag agtgtcccag ccagatgtgt tccctcaccc catgttcatt tacatgtcct |
| 2881 | tcaatgccca cctcaaaagg tacctcttct gtaaagcttt ccctggtatc aggaatcaaa |
| 2941 | attaatcagg gatcttttca cactgctgtt ttttcctctt tggtccttct atcactaaaa |
| 3001 | ctcatctcat tcagccttac agcataacta attatttgtt ttcctcacta cattgtacat |
| 3061 | gtgggaatta cagataaacg gaagccggct ggggtggtgg ctcacgcctg taatcccaac |
| 3121 | actttgggag gccaaggcag gcggatcacc tgaggtcagg agttcgagat tagtctggcc |
| 3181 | aacatggtga aaccccatct ctactaaaaa tacgaaatta gccaggtgtg gtggcacaca |
| 3241 | tctgtagtcc cagctactct ggaggctgag acaggagaat cgcttgaacc caggaagtgg |
| 3301 | aggttgcagt gagctgagat cacaccactg cactccagcc tgggagagac agagtgagac |
| 3361 | tccatctcga aaaaaaaaaa aagatagaag ccaataagca tggtgcaatc aaattctggc |
| 3421 | aagcattaaa tatcaggatg cagctgggca cggtggctca cgcctgtaat cccagcactt |
| 3481 | tgggaggcca aggtgggcgg atcacttgag gtcaggaatt tgagaggatc ctggccagca |
| 3541 | tggcaaaacc ccatctgtac ttaaaataca aaaaaattag ctgggcgtgg tggtgcacac |
| 3601 | ctgtaatccc agctacttgg gaggctgagg tgggagaatt gcttgaacct gggaggtgga |
| 3661 | ggttgcagtg agctgagatc ctgccactgc actccaggct gggcaacaga gtgagaccat |
| 3721 | gtctcaaaaa ataaaaataa aataaaataa tatcaggatg catacatcag aggctgttcc |
| 3781 | tagtgtaaag gcactttgga gggagaagac tttcagagtt aggcagacca actaagaggt |
| 3841 | cagctgaagc acctaaccag ttgtaaggag gtgaaagaca gcaccccaag aagagacgtg |
| 3901 | caggaaggag gaaagaggct tggtcataaa ggatggagga attccaaagt gacactgaac |
| 3961 | aggctgcgtt tatcctaaaa taaaaccact cctcactctg tggatgcgtt ccagactcat |
| 4021 | tcccaaacat ctttattctc taacttgccc tcttcctctt cctaatatgc tcactcaagt |
| 4081 | aaaattacta gtgtcctaat gcccctatgc atattgtcaa aaataaaaat cagaagcagg |
| 4141 | ttagatctgt taggtcttcc agaagagcaa acctgggatg aagccagagc ccaggaattc |
| 4201 | tccaggtagc ctttggactc aggacaccct actcttgtct ctcctatcag tttctctgct |
| 4261 | atgaatctcc tgattcatga acacgttatc tgttcaccct tctctctagg tcttagttct |
| 4321 | tagattttcc ttctgtaaaa tgcatgtgat cttattttcc cctccacaac tttccagatg |
| 4381 | aactagactg tgaccaagag gtctataaaa tcaaagcatc atggaacagg atcttgtatc |
| 4441 | agaccaaagt gtgccagttt ttaaaaatgt gcatcaaaat ggaagtctca gagacagagc |
| 4501 | cctctggtgg aaagttctag taggttagga cagtcctgcc tgcagacacc ttgggcttta |
| 4561 | ctgagggact caactgagaa aatgaggaat gttgcagctc atgattctta gaagaagaaa |
| 4621 | gtgaagcttg tttaaaatat gatttaaaaa atctgtagaa cactgtaaac tacacaggct |
| 4681 | atgagggaat agcctggttg ggccagcttg gaaatcgggc acaggcagga aggggcctgt |
| 4741 | ctggtttggg ccgtgtccac agagagcact tattaggtcc tgcctggaga gaaggaatgg |
| 4801 | ctgggctata ttttcttcca gactcattat ttttcttctg tttgactttt ctctgaattt |
| 4861 | cccttgattt gtataaattt tctcaataat tagtgacagt gtctactgat tgtaaaatga |
| 4921 | agcttgaagg ccaggcgcag tggctcatgc ctgtaatcct agaattttgg gaggccaagg |
| 4981 | tgggtggatc acaaggagtt cgagaccagc ctggccaagg ttgtgaaacc gcgtctctac |
| 5041 | taaaaataca aaaaaattag ccgggcatgg tggcacgtgc ctgtagtccc agctactcag |
| 5101 | gaagctgagg caacagaatc acttgaacct gggaggtgga ggttgcagtg agccgagatc |
| 5161 | acgccactgc actccagcct gggcgagaga gtgagactcc gtctcaaaaa aaaaaaaaaa |
| 5221 | aaaaaaagtt tgaagaacaa agacaataag aggaaatata atgagtggtc ataaatgtgg |
| 5281 | gctctgacag tagagtgcct gggtctgcat cctggtttct tagtcatgtg accttaggca |
| 5341 | agttacttta acctcgctgt acctcaggtt gtccatctgt aaaatgggga taataatagt |
| 5401 | gcctaccttt taaggttgat gtggggatta aatgaggtgt tgctcataca ggaatgtgcc |
| 5461 | tgtgcatggc aaagttcggg aaatttttta taagctgttc taggcctgaa atcttcagaa |
| 5521 | gatgctaatc taaattcatg aaataagctt cttacaacag aaatgctgct agtattatgc |
| 5581 | aaaattaatg ttgtatatca aacttttaac tctcatccct ccttattcag atatattttg |
| 5641 | ttataagcaa tgtttgttcc cctcgttatt ataccacagt ctacttacct gatgctatat |
| 5701 | ctgcctcccc agttagactg agagaacagg ggatatacct aaataataat aataataata |
| 5761 | ataataataa ataataatgg agagctcctt gaagataggg agcctgtaag aatcattgag |
| 5821 | ggcttatttt gtataccaac tgctaaacta gatgcttcat acattgttgt caatactcat |
| 5881 | gacagccttg taaagtagaa attaattctt ccagttaaca ctaaggctga catatgaata |
| 5941 | ccttggcaaa tctggaaagc tgggaagaca gtatttgaat tcaagacttc ttgtcaccaa |
| 6001 | gggccatgca cttgtactct gccatgtggc ccttttttac ctcctgtgga ttctccctac |
| 6061 | ctggtacttg gccttaggtg tacacacacc tggcactttg cttgacacat aataggtgga |
| 6121 | ccacaaatat ctactaaatg aatatttgca tatagtaata ttttaaggta ctaaaagcag |
| 6181 | ctcaaagtaa atatattaat atattaattc cattgctatc tggataacca ctcaactttc |
| 6241 | ctgctgaaaa tgcccattta attaaagaag gttggataga gctctctata tgcattttgg |
| 6301 | acaggcaggg gtttcaggtc ataaacdttc tgatgagtta atataaaata agagaaactg |
| 6361 | taaatttcca ctactaaaaa tcacaaaaat aacagaaaca aaagaagaga taagaatttg |
| 6421 | gggaattgtg ctgaacaatt tagtggttaa aaaaaacaac tgtgcatgtt tagacttaaa |
| 6481 | taagccccca tccaagtgtg aggggtccag taatttttca aaacatatga aagtgttaat |
| 6541 | acatttcgac aaaggaccat taaaaaagtc ctgaattctg acttgaggga ggaaagtaat |
| 6601 | gactaataca ttctctagag acttgcagac tttgggaatt cataaaggaa tggatgataa |
| 6661 | ttattaactg ttgctggctg attgcccaga cagttctcaa cagccctgta caagtctctg |
| 6721 | ggtttgggat ggatcaattc tgagactgga aaatggccaa atctttgcaa atgagaaata |
| 6781 | tttttcttat aagttcttat tgtaggcaaa taattacata gattattcat cagagaattt |
| 6841 | ttaaatgctc ataatctcaa ctctttcatt tacaacttgt atttccaata gtttatgggt |
| 6901 | catctctgca tagatgtcag aagtcacctc aagtttagcg tgtccaaaat ctaactcaca |
| 6961 | ggtctgtttc tgacctccca acttgctttc cttgtgtttt tcctatgcta atgatccacc |
| 7021 | ataatcaaaa taattaacat ttatccagtg cctactatgt actattccct gtcctgtttt |
| 7081 | acatttactc atttaaagtc cataagaaac attaaatctc atctgccttc tgaagaagat |
| 7141 | acaaccatgc tct ctttta aaagtaggaa actgggtcac agaaaggtga agtctttaag |
| 7201 | gctgaatcac agtagctcat cctagtaaat agaaaagcca ggattcaact ccaggggctg |
| 7261 | ggtgcagaac tgctattctt cactgcttca ccaatcagca gctacccaag gcagaaaact |
| 7321 | ttttcatcct tggctccttc attctccctg tcaccccaga tcccctctac atctagtcag |
| 7381 | agaaataggt ctgtcaattc caacttctct atatggctcc tctcaggcat gtgcccttaa |
| 7441 | ttggcctaat tctctaatac accttccctc tacatgctca ctccctcaga tcattgcttt |
| 7501 | atcacgtgtt acctgggttg ctattacata aagagcaatc tttctaaaat gaggatctta |
| 7561 | tcacttcact tccacactaa aatgtttttc ctggggaacc acactcctta gcaatctgac |
| 7621 | ccatcagacc ttccaggctg tctcctgcct gctccctaag gctccagcca cacagaatta |
| 7681 | tcatgggccc acacacccac caaatcctcc catgcctttg cccatgttgt ctgggatgcc |
| 7741 | cttctctccc ttctgtctac atcaagcatc agactgaata tccctcttgt gcggccttct |
| 7801 | aaaacctccc gtccaaagcg aaatatattg ccctctattt atacttttac agcatttggc |
| 7861 | acacaagtac agagtagtag ctttttatca cattctctga taattatata gatatggtat |
| 7921 | ttctatagct tctctccaac tggctaataa gttgcttttt gtctgagtgc ctaattttgt |
| 7981 | gttttgtgtc tgagtgcctc agttcctcaa aaaaaggttt tttgattagt tcattattca |
| 8041 | tttgaacatg gaaattatgc tcactagtgg caaatgccac taaccgtatt ccagaagcta |
| 8101 | ggtgtcatgt ttgcaataag atatattatc ccttctacaa gtcacctttt atttcaggca |
| 8161 | tttgtaaatg cccattaata aagtatggtt cataaatttt accttgtaag tgcctaagaa |
| 8221 | atgagactac aagctccatt tcagcaggac acaataaata ttattttata atgcatctaa |
| 8281 | aaaaaaaaaa aaaaaa |

SEQ ID NO: 54 Human CD84 Isoform 1 Amino Acid Sequence (NP_001171808.1)

| | |
|---|---|
| 1 | maqhhlwill lclqtwpeaa gkdseiftvn gilgesvtfp vniqeprqvk iiawtsktsv |
| 61 | ayvtpqdset apvvtvthrn yyerihalgp nynivisdlr medagdykad intqadpytt |
| 121 | tkrynlqiyr rlgkpkitqs lmasvnstcn vtltcsveke eknvtynwsp lgeegnvlqi |
| 181 | fqtpedqelt ytctaqnpvs nnsdsisarq lcadiamgfr thhtgllsvl amffllvlil |
| 241 | ssvflfrlfk rrqgrifpeg sclntftknp yaaskktiyt yimasrntqp aesriydeil |
| 301 | qskvlpskee pvntvysevq fadkmgkast qdskppgtss yeivi |

SEQ ID NO: 55 Human CD84 Transcript Variant 2 cDNA Sequence (NM_003874.3;
CDS: 80-1066)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctggccg gaagcagctg gaaaagactc agaaatcttc acagtgaatg ggattctggg |
| 181 | agagtcagtc actttccctg taaatatcca agaaccacgg caagttaaaa tcattgcttg |
| 241 | gacttctaaa acatctgttg cttatgtaac accaggagac tcagaaacag cacccgtagt |
| 301 | tactgtgacc cacagaaatt attatgaacg gatacatgcc ttaggtccga actacaatct |
| 361 | ggtcattagc gatctgagga tggaagacgc aggagactac aaagcagaca taaatacaca |
| 421 | ggctgatccc tacaccacca ccaagcgcta caacctgcaa atctatcgtc ggcttgggaa |
| 481 | accaaaaatt acacagagtt taatggcatc tgtgaacagc acctgtaatg tcacactgac |
| 541 | atgctctgta gagaaagaag aaaagaatgt gacatacaat tggagtcccc tgggagaaga |
| 601 | gggtaatgtc cttcaaatct tccagactcc tgaggaccaa gagctgactt acacgtgtac |
| 661 | agcccagaac cctgtcagca acaattctga ctccatctct gcccggcagc tctgtgcaga |
| 721 | catcgcaatg ggcttccgta ctcaccacac cgggttgctg agcgtgctgg ctatgttctt |
| 781 | tctgcttgtt ctcattctgt cttcagtgtt tttgttccgt ttgttcaaga gaagacaaga |
| 841 | tgctgcctca aagaaaacca tatacacata tatcatggct tcaaggaaca cccagccagc |
| 901 | agagtccaga atctatgatg aaatcctgca gtccaaggtg cttccctcca aggaagagcc |
| 961 | agtgaacaca gtttattccg aagtgcagtt tgctgataag atggggaaag ccagcacaca |
| 1021 | ggacagtaaa cctcctggga cttcaagcta tgaaattgtg atctaggctg ctgggctgaa |
| 1081 | ttctccctct ggaaactgag ttacaaccac caatactggc aggttccctg gatccagatc |
| 1141 | ttctctgccc aactcttact gggagattgc aaactgccac atctcagcct gtaagcaaag |
| 1201 | caggaaacct tctgctgggc atagcttgtg cctaaatgga caaatggatg catacccttc |
| 1261 | ctgaaatgac tccctatctg atgaatgaca aagcaggtta cctagtatag ttttcccaaa |
| 1321 | cttcttccca taatagcaca tgtagaaaat aatattttta tggcacactg ggataaacaa |
| 1381 | gcaagattgc taacttctgg aagctgcata tgactagagg cctcttgtga ctggaggtaa |
| 1441 | caaccctgcc cagtaactgt gggagaaggg gatcaatatt ttgcacacct gtaataggcc |
| 1501 | atggcacacc agccaagatg ctctgctcac agtcagtatg tgtgaagatc cctggtgcgt |
| 1561 | ggccttcacc acgcatcttg agcaaattag gaaaatgtac cacttcgctt aggcagatgc |
| 1621 | agcccttccc ccgagtgcat ggcttggaga gcagaatgtg ggctgcatat aagcacactc |
| 1681 | atccctttgt ctgggaatct ttgtgcaggg cataacaggc ttagtaagtc caaacacaga |
| 1741 | tgacagtgct gtgtgggtct ctgtcagagt tgtggctctc agccatgtag acacactctc |
| 1801 | caaatggagt gttggaaaat gttctttctg cagggtctag agactgctgg gacacttttc |
| 1861 | ttggagtgct acttcagaag ccttatagga ttttctttct ggccaagatt tccttctgta |
| 1921 | tcactccaag cagcctcagc agaaccagca gccatgccca gtattcccac tctccaaaag |
| 1981 | gaactgacca gcttatattt ctcacacttc tggggaactg ggtataatcc aaccatcaaa |
| 2041 | atagaagacc ttgcaagaag cagagtcatt ctccagaagg aacttgggag atgatggtgc |
| 2101 | agatgatgaa actgggttca tcccagttcc aaagactcag agaactagag tttaagctga |
| 2161 | ggcagagtgc cgccaccctg gcatgcccca caaacagatc accagccagc ttacacaggc |
| 2221 | attaactctc ctcaatgagg aagaatcatt cacaactgag caagacattc atatgatcat |
| 2281 | ttaaggaagt gtttccctta tgtgttagca agtataatcg gctaactcct aaatcccaat |
| 2341 | gaatagtcct aggctggaca gcaatgggct gcaattaggc agataaagac atcagtccca |
| 2401 | gtaaatgaat ccatagactc atctagcacc aactaccatt agcactatgt taggagctgc |
| 2461 | aaggccccaa agtagaagat gtgcataatg tctgctcttg tgtagctcag gagacaattc |
| 2521 | cagcacagac actacagtta acgctgaact gcagctgcaa gtaatagcat gaacagtcag |
| 2581 | aaaaatacct tatgaggggg cagggctgaa gctgggcctt gaaggatgga tgaaatttgg |
| 2641 | atagagaatg aggaagacag agggcctcca agtgagagaa gcatgaaaaa tgagcagggg |
| 2701 | cctggatcag tggggtgtat tcagagcacc tctccagatg caccatgcat gctcacagtc |
| 2761 | ccttgcctat gtgtggcaga gtgtcccagc cagatgtgtg ccctcacccc atgtccattt |
| 2821 | acatgtcctt caatgcccac ctcaaaaggt acctcttctg taaagctttc cctggtatca |
| 2881 | ggaatcaaaa ttaatcaggg atcttttcac actgctgttt tttcctcttt ggtccttcta |
| 2941 | tcactaaaac tcatctcatt cagccttaca gcataactaa ttatttgttt tcctcactac |
| 3001 | attgtacatg tgggaattac agataaacgg aagccggctg gggtggtggc tcacgcctgt |
| 3061 | aatcccaaca ctttgggagg ccaaggcagg cggatcacct gaggtcagga gttcgagatt |
| 3121 | agtctggcca acatggtgaa accccatctc tactaaaaat acgaaattag ccaggtgtgg |
| 3181 | tggcacacat ctgtagtccc agctactctg gaggctgaga caggagaatc gcttgaaccc |
| 3241 | aggaagtgga ggttgcagtg agctgagatc acaccactgc actccagcct gggagagaca |
| 3301 | gagtgagact ccatctcgaa aaaaaaaaaa agatagaagc caataagcat ggtgcaatca |
| 3361 | aattctggca agcattaaat atcaggatgc agctgggcac ggtggctcac gcctgtaatc |
| 3421 | ccagcacttt gggaggccaa ggtgggcgga tcacttgagg tcaggaattt gagaggatcc |
| 3481 | tggccagcat ggcaaaaccc catctgtact taaaatacaa aaaaattagc tgggcgtggt |
| 3541 | ggtgcacacc tgtaatccca gctacttggg aggctgaggt gggagaattg cttgaacctg |
| 3601 | ggaggtggag gttgcagtga gctgagatcc tgccactgca ctccaggctg ggcaacagag |
| 3661 | tgagaccatg tctcaaaaaa taaaaataaa ataaaataat atcaggatgc atacatcaga |
| 3721 | ggctgttcct agtgtaaagg cactttggag ggagaagact ttcagagtta ggcagaccaa |
| 3781 | ctaagaggtc agctgaagca cctaaccagt tgtaaggagg tgaaagacag caccccaaga |
| 3841 | agagacgtgc aggaaggagg aaagaggctt ggtcataaag gatggaggaa ttccaaagtg |
| 3901 | acactgaaca ggctgcgttt atcctaaaat aaaaccactc ctcactctgt ggatgcgttg |
| 3961 | aagactcatt cccaaacatc tttattctct aacttgccct cttcctcttc ctaatatgct |
| 4021 | cactcaagta aaattactag tgtcctaatg cccctatgca tattgtcaaa aataaaaatc |
| 4081 | agaagcaggt tagatctgtt aggtcttcca gaagagcaaa cctgggatga agccagagcc |
| 4141 | caggaattct gaaggtagcc tttggactca ggacacccta ctcttgtctc tcctctcagt |
| 4201 | ttctctgcta tgaatctcct gattcatgaa cacgttatct gttcaccctt ctctctaggt |
| 4261 | cttagttctt agattttcct tctgtaaaat gcatgtgatc ttattttccc ctccacaact |
| 4321 | ttccagatga actagactgt gaccaagagg tctataaaat caaagcatca tggaacagga |
| 4381 | tcttgtatca gaccaaagtg tgccagtttt taaaaatgtg catcaaaatg gaagtctcag |
| 4441 | agacagagcc ctctggtgga aagttctagt aggttaggac agtcctgcct gcagacacct |
| 4501 | tgggctttac tgagggactc aactgagaaa atgaggaatg ttgcagctca tgattcttag |
| 4561 | aagaagaaag tgaagcttgt ttaaaatatg atttaaaaaa tctgtagaac actgtaaact |
| 4621 | acacaggcta tgagggaata gcctggttgg gccagcttgg aaatcgggca caggcdggaa |
| 4681 | ggggcctgtc tggtttgggc cgtgtccaca gagagcactt cttaggtcct gcctggagag |
| 4741 | aaggaatggc tgggctatat tttcttccag actcattatt tttcttctgt ttgacttttc |
| 4801 | tctgaatttc ccttgatttg tataaatttt ctcaataatt agtgacagtg tctactgatt |
| 4861 | gtaaaatgaa gcttgaaggc caggcgcagt ggctcatgcc tgtaatccta gaattttggg |
| 4921 | aggccaaggt gggtggatca caaggagttc gagaccagcc tggccaaggt tgtgaaaccg |
| 4981 | cgtctctact aaaaatacaa aaaaattagc cgggcatggt ggcacgtgcc tgtagtccca |
| 5041 | gctactcagg aagctgaggc aacagaatca cttgaacctg ggaggtggag gttgcagtga |
| 5101 | gccgagatca cgccactgca ctccagcctg ggcgagagag tgagactccg tctcaaaaaa |
| 5161 | aaaaaaaaaa aaaaaagttt gaagaacaaa gacaataaga ggaaatataa tgagtggtca |
| 5221 | taaatgtggg ctctgacagt agagtgcctg ggtctgcatc ctggtttctt agtcatgtga |
| 5281 | ccttaggcaa gttactttaa cctcgctgta cctcaggttg tccatctgta aaatggggat |
| 5341 | aataatagtg cctacctttt aaggttgatg tggggattaa atgaggtgtt gctcatacag |
| 5401 | gaatgtgcct gtgcatggca aagttcggga aattttttat aagctgttct aggcctgaaa |
| 5461 | tcttcagaag atgctaatct aaattcatga aataagcttc ttacaacaga aatgctgcta |
| 5521 | gtattatgca aaattaatgt tgtatatcaa acttttaact ctcatccctc cttattcaga |
| 5581 | tatattttgt tataagcaat gtttgttccc ctcgttatta taccacagtc tacttacctg |
| 5641 | atgctatatc tgcctcccca gttagactga gagaacaggg gatataccta aataataata |
| 5701 | ataataataa taatactaaa taataatgga gagctccttg aagataggga gcctgtaaga |
| 5761 | atcattgagg gcttattttg tatagcaact gctaaactag atgcttcata cattgttgtc |
| 5821 | aatactcatg acagccttgt aaagtagaaa ttaattcttc cagttaacac taaggctgac |
| 5881 | atatgaatac cttggcaaat ctggaaagct gggaagacag tatttgaatt caagacttct |
| 5941 | tgtcaccaag ggccatgcac ttgtactctg ccatgtggcc cttttttacc tcctgtggat |
| 6001 | tctccctacc tggtacttgg ccttaggtgt acacacacct ggcactttgc ttgacacata |
| 6061 | ataggtggac cacaaatatc tactaaatga atatttgcat atagtaatat tttaaggtac |
| 6121 | taaaagcagc tcaaagtaaa tatattaata tattaattcc attgctatct ggataaccac |
| 6181 | tcaactttcc tgctgaaaat gcccatttaa ttaaagaagg ttggatagag ctctctatat |
| 6241 | gcattttgga caggcagggg tttcaggtca taaacattct gatgagttaa tataaaataa |
| 6301 | gagaaactgt aaatttccac tactaaaaat cacaaaaata acagaaacaa aagaagagat |
| 6361 | aagaatttgg ggaattgtgc tgaacaattt agtggttaaa aaaaacaact gtgcatgttt |
| 6421 | agacttaaat aagcccccat ccaagtgtga ggggtccagt aatttttcaa aacatatgaa |
| 6481 | agtgttaata catttcgaca aaggaccatt aaaaaagtcc tgaattctga cttgagggag |
| 6541 | gaaagtaatg actaatacat tctctagaga cttgcagact ttgggaattc ataaaggaat |
| 6601 | ggatgataat tattaactgt tgctggctga ttgcccagac agttctcaac agccctgtac |
| 6661 | aagtctctgg gtttgggatg gatcaattct gagactggaa aatggccaaa tctttgcaaa |
| 6721 | tgagaaatat ttttcttata agttcttatt gtaggcaaat aattacatag attattcatc |
| 6781 | agagaatttt taaatgctca taatctcaac tctttcattt acaacttgta tttccaatag |
| 6841 | tttatgggtc atctctgcat agatgtcaga agtcacctca agtttagcgt gtccaaaatc |
| 6901 | taactcacag gtctgtttct gacctcccaa cttgctttcc ttgtgttttt cctatgctaa |
| 6961 | tgatccacca taatcaaaat aattaacatt tatccagtgc ctactatgta ctattccctg |
| 7021 | tcctgtttta catttactca tttaaagtcc ataagaaaca ttaaatctca tctgccttct |
| 7081 | gaagaagata caaccatgct ctcttttaca aagtaggaaa ctgggtcaca gaaaggtgaa |
| 7141 | gtctttaagg ctgaatcaca gtagctcatc ctagtaaata gaaaagccag gattcaactc |
| 7201 | caggggctgg gtgcagaact gctattcttc actgcttcac caatcagcag ctacccaagg |
| 7261 | cagaaaactt tttcatcatt ggctccttca ttctccctgt caccccagat cccctctaca |
| 7321 | tctagtcaga gaataggtcc tgtcaattcc aacttctcta tatggctcct ctcaggcatg |
| 7381 | tgcccttaat tggcctaatt ctctaatgca ccttccctct acatgctcac tccctcagat |
| 7441 | cattgcttta tcacgtgtta cctgggttgc tattacataa agagcaatct ttctaaaatg |
| 7501 | aggatcttat cacttcactt ccacactaaa atgtttttcc tggggaacca cactccttag |
| 7561 | caatctgacc catcagacct tccaggctgt ctcctgcctg ctccctaagg ctccagccac |
| 7621 | acagaattat catgggccca cacacccacc aaatcctccc atgcctttgc ccatgttgtc |
| 7681 | tgggatgccc ttctctccct tctgtctaca tcaagcatca gactgaatat ccctcttgtg |
| 7741 | cggccttcta aaacctcccg tccaaagcga aatatattgc cctctattta tacttttaca |
| 7801 | gcatttggca cacaagtaca gagtagtagc tttttatcac attctctgat aattatatag |
| 7861 | atatggtatt tcttagctct ctctccaact ggctaataag ttgctttttg tctgagtgcc |
| 7921 | taattttgtg ttttgtgtct gagtgcctca gttcctcaaa aaaaggtttt ttgattagtt |
| 7981 | cattattcat ttgaacatgg aaattatgct cactagtggc aaatgccact aaccgtattc |
| 8041 | cagaagctag gtgtcatgtt tgcaataaga tatattatcc cttctacaag tcacctttta |
| 8101 | tttcaggcat ttgtaaatgc ccattaataa agtatggttc ataaatttta ccttgtaagt |
| 8161 | gcctaagaaa tgagactaca agctccattt cagcaggaca caataaatat tattttataa |
| 8221 | tgcatctaaa aaaaaaaaaa aaaaa |

SEQ ID NO: 56 Human CD84 Isoform 2 Amino Acid Sequence (NP_003865.1)

| | |
|---|---|
| 1 | maqhhlwill lclqtwpeaa gkdseiftvn gilqesvtfp vniqeprqvk iiawtsktsv |
| 61 | ayvtpgdset apvvtvthrn yyerihalgp nynlvisdlr medagdykad intqadpytt |
| 121 | tkryniqiyr rlgkpkitqs lmasvnstcn vtltcsveke eknvtynwsp lgeegnvlqi |
| 181 | fqtpedqelt ytctaqnpvs nnsdsisarq lcadiamgfr thhtgllsvl amffllvlil |
| 241 | ssvflfrlfk rrqdaaskkt iytyimasrn tqpaesriyd eilqskvlps keepvntvys |
| 301 | evgfadkmgk astqdskppg tssyeivi |

SEQ ID NO: 57 Human CD84 Transcript Variant 3 cDNA Sequence
(NM_001184881.1; CDS: 80-898)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctggccg gaagcagctg gaaaagactc agaaatcttc acagtgaatg ggattctggg |
| 181 | agagtcagtc actttccctg taaatatcca agaaccacgg caagttaaaa tcattgcttg |
| 241 | gacttctaaa acatctgttg cttatgtaac accaggagac tcagaaacag cacccgtagt |
| 301 | tactgtgacc cacagaaatt attatgaacg gatacatgcc ttaggtccga actacaatct |
| 361 | ggtcattagc gatctgagga tggaagacgc aggagactac aaagcagaca taaatacaca |
| 421 | ggctgatccc tacaccacca ccaagcgcta caacctgcaa atctatcgtc ggcttgggaa |
| 481 | accaaaaatt acacagagtt taatggcatc tgtgaacagc acctgtaatg tcacactgac |
| 541 | atgctctgta gagaaagaag aaaagaatgt gacatacaat tggagtcccc tgggagaaga |
| 601 | gggtaatgtc cttcaaatct tccagactcc tgaggaccaa gagctgactt acacgtgtac |
| 661 | agcccagaac cctgtcagca acaattctga ctccatctct gcccggcagc tctgtgcaga |
| 721 | catcgcaatg ggcttccgta ctcaccacac cgggttgctg agcgtgctgg ctatgttctt |
| 781 | tctgcttgtt ctcattctgt cttcagtgtt tttgttccgt ttgttcaaga gaagacaagg |
| 841 | tgcttccctc caaggaagag ccagtgaaca cagtttattc cgaagtgcag tttgctgata |
| 901 | agatggggaa agccagcaca caggacagta aacctcctgg gacttcaagc tatgaaattg |
| 961 | tgatctaggc tgctgggctg aattctccct ctggaaactg agttacaacc accaatactg |
| 1021 | gcaggttccc tggatccaga tcttctctgc ccaactctta ctgggagatt gcaaactgcc |
| 1081 | acatctcagc ctgtaagcaa agcaggaaac cttctgctgg gcatagcttg tgcctaaatg |
| 1141 | gacaaatgga tgcataccct tcctgaaatg actcccttct gaatgaatga caaagcaggt |
| 1201 | tacctagtat agttttccca aacttcttcc catcatagca catgtagaaa ataatatttt |
| 1261 | tatggcacac tgggataaac aagcaagatt gctcacttct ggaagctgca tatgactaga |
| 1321 | ggcctcttgt gactggaggt aacaaccctg cccagtaact gtgggaccag gggatcaata |
| 1381 | ttttgcacac ctgtaatagg ccatggcaca ccagccaaga tgctctgctc acagtcagta |
| 1441 | tgtgtgaaga tccctggtgc gtggccttca ccacgcatct tgagcaaatt aggaaaatgt |
| 1501 | acccttcgct tgaggcagat gcagccactt ccccgagtgc atggcttgga gagcagaatg |
| 1561 | tgggctgcat ataagcacac tcatcccttt gtctgggaat ctttgtgcag ggcataacag |
| 1621 | gcttagtaag tccaaacaca gatgacagtg ctgtgtgggt ctctgtcaga gttgtggctc |
| 1681 | tcagccatgt agacacactc tccaaatgga gtgttggaaa atgttctttc tgcagggtct |
| 1741 | agagactgct gggacacttt tcttggagtg ctacttcaga agccttatag gattttcttt |
| 1801 | ctggccaaga tttccttctg tatcactcca agcagcctca gcagaagaag cagccatgcc |
| 1861 | cagtattccc actctccaaa aggaactgac cagcttatat ttctcacact tctggggaac |
| 1921 | tgggtataat ccaaccatca aaatagaaga ccttgcaaga agcagagtca ttctccagaa |
| 1981 | ggaacttggg agatgatggt gcagatgatg aaactgggtt catcccagtt ccaaagactc |
| 2041 | agagaactag agtttaagct gaggcagagt gccgccaccc tggcatgccc cacaaacaga |
| 2101 | tcaccagcca gcttacacag gcattaactc tcctcaatga ggaagaatca ttcacaactg |
| 2161 | agcaagacat tcatatgatc atttaaggaa gtgtttccct tatgtgttag caagtataat |
| 2221 | cggctaactc ctaaatccca atgaatagtc ctaggctgga cagcaatggg ctgcaattag |
| 2281 | gcagataaag acatcagtcc cagtaaatga atccatagac tcatctagca ccaactacca |
| 2341 | ttagcactat gttaggagct gcaaggcccc aaagtagaag atgtgcataa tgtctgctct |
| 2401 | tgtgtagctc aggagacaat tccagcacag acactacagt taacgctgaa ctgcagctgc |
| 2461 | aagtaatagc atgaacagtc agaaaaatac cttatgaggg ggcagggctg aagctgggcc |
| 2521 | ttgaaggatg gatgaaattt ggatagagaa tgaggaagac agagggcctc caagtgagag |
| 2581 | aagcatgaaa aatgagcagg ggcctggatc agtggggtgt attcagagca cctctccaga |
| 2641 | tgcaccatgc atgctcacag tcccttgcct atgtgtggca gagtgtccca gccagatgtg |
| 2701 | tgccctcacc ccatgtccat ttacatgtcc ttcaatgccc acctcaaaag gtacctcttc |
| 2761 | tgtaaagctt tccctggtat caggaatcaa aattaatcag ggatcttttc acactgctgt |
| 2821 | tttttcctct ttggtccttc tatcactaaa actcatctca ttcagcctta cagcataact |
| 2881 | aattatttgt tttcctcact acattgtaca tgtgggaatt acagataaac ggaagccggc |
| 2941 | tggggtggtg gctcacgcct gtaatcccaa cactttggga ggccaaggca ggcggatcac |
| 3001 | ctgaggtcag gagttcgaga ttagtctggc caacatggtg aaaccccatc tctactaaaa |
| 3061 | atacgaaatt agccaggtgt ggtggcacac atctgtagtc ccagctactc tggaggctga |
| 3121 | gacaggagaa tcgcttgaac ccaggaagtg gaggttgcag tgagctgaga tcacaccact |
| 3181 | gcactccagc ctgggagaga cagagtgaga ctccatctcg aaaaaaaaaa aaagatagaa |
| 3241 | gccaataagc atggtgcaat caaattctgg caagcattaa atatcaggat gcagctgggc |
| 3301 | acggtggctc acgcctgtaa tcccagcact ttgggaggcc aaggtgggcg gatcacttga |
| 3361 | ggtcaggaat ttgagaggat cctggccagc atggcaaaac cccatctgta cttaaaatac |
| 3421 | aaaaaaatta gctgggcgtg gtggtgcaca cctgtaatcc cagctacttg ggaggctgag |
| 3481 | gtgggagaat tgcttgaacc tgggaggtgg aggttgcagt gagctgagat cctgccactg |
| 3541 | cactccaggc tgggcaacag agtgagacca tgtctcaaaa aataaaaata aaataaaata |
| 3601 | atatcaggat gcatacatca gaggctgttc ctagtgtaaa ggcactttgg agggagaaga |
| 3661 | ctttcagagt taggcagacc aactaagagg tcagctgaag cacctaacca gttgtaagga |
| 3721 | ggtgaaagac agcaccccaa gaagagacgt gcaggaagga ggaaagaggc ttggtcataa |
| 3781 | aggatggagg aattccaaag tgacactgaa caggctgcgt ttatcctaaa ataaaaccac |
| 3841 | tcctcactct gtggatgcgt tgaagactca ttcccaaaca tctttattct ctaacttgcc |
| 3901 | ctcttcctct tcctaatatg ctcactcaag taaaattact agtgtcctaa tgcccctatg |
| 3961 | catattgtca aaaataaaaa tcagaagcag gttagatctg ttaggtcttc cagaagagca |
| 4021 | aacctgggat gaagccagag cccaggaatt ctgaaggtag cctttggact caggacaccc |
| 4081 | tactcttgtc tctcctctca gtttctctgc tatgaatctc ctgattcatg aacacgttat |
| 4141 | ctgttcaccc ttctctctag gtcttagttc ttagattttc cttctgtaaa atgcatgtga |
| 4201 | tcttattttc ccctccacaa ctttccagat gaactagact gtgaccaaga ggtctataaa |
| 4261 | atcaaagcat catggaacag gatcttgtat cagaccaaag tgtgccagtt tttaaaaatg |
| 4321 | tgcatcaaaa tggaagtctc agagacagag ccctctggtg gaaagttcta gtaggttagg |
| 4381 | acagtcctgc ctgcagacac cttgggcttt actgagggac tcaactgaga aaatgaggaa |
| 4441 | tgttgcagct catgattctt agaagaagaa agtgaagctt gtttaaaata tgatttaaaa |
| 4501 | aatctgtaga acactgtaaa ctacacaggc tatgagggaa tagcctggtt gggccagctt |
| 4561 | ggaaatcggg cacaggcagg aaggggcctg tctggtttgg gccgtgtcca cagagagcac |
| 4621 | ttcttaggtc ctgcctggag agaaggaatg gctgggctat attttcttcc agactcatta |
| 4681 | tttttcttct gtttgacttt tctctgaatt tcccttgatt tgtataaatt ttctcaataa |
| 4741 | ttagtgacag tgtctactga ttgtaaaatg aagcttgaag gccaggcgca gtggctcatg |
| 4801 | cctgtaatcc tagaattttg ggaggccaag gtgggtggat cacaaggagt tcgagaccag |
| 4861 | cctggccaag gttgtgaaac cgcgtctcta ctaaaaatac aaaaaaatta gccgggcatg |
| 4921 | gtggcacgtg cctgtagtcc cagctactca ggaagctgag gcaacagaat cacttgaacc |
| 4981 | tgggaggtgg aggttgcagt gagccgagat cacgccactg cactccagcc tgggcgagag |
| 5041 | agtgagactc cgtctcaaaa aaaaaaaaaa aaaaaaaagt ttgaagaaca aagacaataa |
| 5101 | gaggaaatat aatgagtggt cataaatgtg ggctctgaca gtagagtgcc tgggtctgca |
| 5161 | tcctggtttc ttagtcatgt gaccttaggc aagttacttt aacctcgctg tacctcaggt |
| 5221 | tgtccatctg taaaatgggg ataataatag tgcctacctt ttaaggttga tgtggggatt |
| 5281 | aaatgaggtg ttgctcatac aggaatgtgc ctgtgcatgg caaagttcgg gaaatttttt |
| 5341 | ataagctgtt ctaggcctga aatcttcaga agatgctaat ctaaattcat gaaataaggt |
| 5401 | tcttacaaca gaaatgctgc tagtattatg caaaattaat gttgtatatc aaacttttaa |
| 5461 | ctctcatccc tccttattca gatatatttt gttataagca atgtttgttc ccctcgttat |
| 5521 | tataccacag tctacttacc tgatgctata tctgcctccc cagttagact gagagaacag |
| 5581 | gggatatacc taaataataa taataataat aataataata aataataatg gagagctcct |
| 5641 | tgaagatagg gagcctgtaa gaatcattga gggcttattt tgtataccaa ctgctaaact |
| 5701 | agatgcttca tacattgttg tcaatactca tgacagcctt gtaaagtaga aattaattct |
| 5761 | tccagttaac actaaggctg acatatgaat accttggcaa atctggaaag ctgggaagac |
| 5821 | agtatttgaa ttcaagactt cttgtcacca agggccatgc acttgtactc tgccatgtgg |
| 5881 | ccctttttta cctcctgtgg attctcccta cctggtactt ggccttaggt gtacacacac |
| 5941 | ctggcacttt gcttgacaca taataggtgg accacaaata tctactaaat gaatatttgc |
| 6001 | atatagtaat attttaaggt actaaaagca gctcaaagta aatatattaa tatattaatt |
| 6061 | ccattgctat ctggataacc actcaacttt cctgctgaaa atgcccattt aattaaagaa |
| 6121 | ggttggatag agctctctat atgcattttg gacaggcagg ggtttcaggt cataaacatt |
| 6181 | ctgatgagtt aatataaaat aagagaaact gtaaatttcc actactaaaa atcacaaaaa |
| 6241 | taacagaaac aaaagaagag ataagaattt ggggaattgt gctgaacaat ttagtggtta |
| 6301 | aaaaaaacaa ctgtgcatgt ttagacttaa ataagccccc atccaagtgt gaggggtcca |
| 6361 | gtaatttttc aaaacatatg aaagtgttaa tacatttcga caaaggacca ttaaaaaagt |
| 6421 | cctgaattct gacttgaggg aggaaagtaa tgactaatac attctctaga gacttgcaga |
| 6481 | ctttgggaat tcataaagga atggatgata attattaact gttgctggct gattgcccag |
| 6541 | acagttctca acagccctgt acaagtctct gggtttggga tggatcaatt ctgagactgg |
| 6601 | aaaatggcca aatctttgca aatgagaaat atttttctta taagttctta ttgtaggcaa |
| 6661 | ataattacat agattattca tcagagaatt tttaaatgct cataatctca actctttcat |
| 6721 | ttacaacttg tatttccaat agtttatggg tcatctctgc atagatgtca gaagtcacct |
| 6781 | caagtttagc gtgtccaaaa tctaactcac aggtctgttt ctgacctccc aacttgcttt |
| 6841 | ccttgtgttt ttcctatgct aatgatccac cataatcaaa ataattaaca tttatccagt |
| 6901 | gcctactatg tactattccc tgtcctgttt tacatttact catttaaagt ccataagaaa |
| 6961 | cattaaatct catctgcctt ctgaagaaga tacaaccatg ctctctttta caaagtagga |
| 7021 | aactgggtca cagaaaggtg aagtctttaa ggctgaatca cagtagctca tcctagtaaa |
| 7061 | tagaaaagcc aggattcaac tccaggggct gggtgcagaa ctgctattct tcactgcttc |
| 7141 | accaatcagc agctacccaa ggcagaaaac tttttcatcc ttggctcctt cattctccct |
| 7201 | gtcaccccag atcccctcta catctagtca gagaataggt cctgtcaatt ccaacttctc |
| 7261 | tatatggctc ctctcaggca tgtgccctta attggcctaa ttctctaata caccttccct |
| 7321 | ctacatgctc actccctcag atcattgctt tatcacgtgt tacctgggtt gctattacat |
| 7381 | aaagagcaat ctttctaaaa tgaggatctt atcacttcac ttccacacta aaatgttttt |
| 7441 | cctggggaac cacactcctt agcaatctga cccatcagac cttccaggct gtctcctgcc |
| 7501 | tgctccctaa ggctccagcc acacagaatt atcatgggcc cacacaccca ccaaatcctc |
| 7561 | ccatgccttt gcccatgttg tctgggatgc ccttctctcc cttctgtcta catcaagcat |
| 7621 | cagactgaat atccctcttg tgcggccttc taaaacctcc cgtccaaagc gaaatatatt |
| 7681 | gccctctatt tatactttta cagcatttgg cacacaagta cagagtagta gctttttatc |
| 7741 | acattctctg ataattatat agatatggta tttcttagct ctctctccaa ctggctaata |
| 7801 | agttgctttt tgtctgagtg cctaattttg tgttttgtgt ctgagtgcct cagttcctca |
| 7861 | aaaaaaggtt ttttgattag ttcattattc atttgaacat ggaaattatg ctcactagtg |
| 7921 | gcaaatgcca ctaaccgtat tccagaagct aggtgtcatg tttgcaataa gatatattat |
| 7981 | cccttctaca agtcaccttt tatttcaggc atttgtaaat gcccattaat aaagtatggt |
| 8041 | tcataaattt taccttgtaa gtgcctaaga aatgagacta caagctccat ttcagcagga |
| 8101 | cacaataaat attattttat aatgcatcta aaaaaaaaaa aaaaaaa |

SEQ ID NO: 58 Human CD84 Isoform 3 Amino Acid Sequence (NP_001171810.1)

| | |
|---|---|
| 1 | maqhhlwill lclqtwpeaa gkdseiftyn gilgesvtfp vniqeprqvk iiawtsktsv |
| 61 | ayvtpgdset apvvtvthrn yyerihalgp nynlvisdlr medagdykad intqadpytt |
| 121 | tkrynlqiyr rlgkpkitqs lmasvnstcn vtltcsveke eknvtynwsp lgeegnvlqi |
| 161 | fqtpedqelt ytctaqnpvs nnsdsisarq lcadiamgfr thhtgllsvl amffllvlil |
| 241 | ssvflfrlfk rrqgaslqgr asehslfrsa vc |

SEQ ID NO: 59 Human CD84 Transcript Variant 4 cDNA Sequence
(NM_001184882.1; CDS: 80-724)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctgtcgg cttgggaaac caaaaattac acagagttta atggcatctg tgaacagcac |
| 161 | ctgtaatgtc acactgacat gctctgtaga gaaagaagaa aagaatgtga catacaattg |
| 241 | gagtcccctg ggagaagagg gtaatgtcct tcaaatcttc cagactcctg aggaccaaga |
| 301 | gctgacttac acgtgtacag cccagaaccc tgtcagcaac aattctgact ccatctctgc |
| 361 | ccggcagctc tgtgcagaca tcgcaatggg cttccgtact caccacaccg ggttgctgag |
| 421 | cgtgctggct atgttctttc tgcttgttct cattctgtct tcagtgtttt tgttccgttt |
| 481 | gttcaagaga agacaagatg ctgcctcaaa gaaaaccata tacacatata tcatggcttc |
| 541 | aaggaacacc cagccagcag agtccagaat ctatgatgaa atcctgcagt ccaaggtgct |
| 601 | tccctccaag gaagagccag tgaacacagt ttattccgaa gtgcagtttg ctgataagat |
| 661 | ggggaaagcc agcacacagg acagtaaacc tcctgggact tcaagctatg aaattgtgat |
| 721 | ctaggctgct gggctgaatt ctccctctgg aaactgagtt acaaccacca atactggcag |
| 761 | gttccctgga tccagatctt ctctgcccaa ctcttactgg gagattgcaa actgccacat |
| 641 | ctcagcctgt aagcaaagca ggaaaccttc tgctgggcat agcttgtgcc taaatggaca |
| 901 | aatggatgca tacccttcct gaaatgactc ccttctgaat gaatgacaaa gcaggttacc |
| 961 | tagtatagtt ttcccaaact tcttcccatc atagcacatg tagaaaataa tatttttatg |
| 1021 | gcacactggg ataaacaagc aagattgctc acttctggaa gctgcatatg actagaggcc |
| 1081 | tcttgtgact ggaggtaaca accctgccca gtaactgtgg gagaagggga tcaatatttt |
| 1141 | gcacacctgt aataggccat ggcacaccag ccaagatgct ctgctcacag tcagtatgtg |
| 1201 | tgaagatccc tggtgcgtgg ccttcaccac gcatcttgag caaattagga aaatgtaccc |
| 1261 | ttcgcttgag gcagatgcag cccttccccc gagtgcatgg cttggagagc agaatgtggg |
| 1321 | ctgcatataa gcacactcat ccctttgtct gggaatcttt gtgcagggca taacaggctt |
| 1381 | agtaagtcca aacacagatg acagtgctgt gtgggtctct gtcagagttg tggctctcag |
| 1441 | ccatgtagac acactctcca aatggagtgt tggaaaatgt tctttctgca gggtctagag |
| 1501 | actgctggga cacttttctt ggagtgctac ttcagaagcc ttataggatt ttctttctgg |
| 1561 | ccaagatttc cttctgtatc actccaagca gcctcagcag aaccagcagc catgcccagt |
| 1621 | attcccactc tccaaaagga actgaccagc ttatatttct cacacttctg gggaactggg |
| 1681 | tataatccaa ccatcaaaat agaagacctt gcaagaagca gagtcattct ccagaaggaa |
| 1741 | cttgggagat gatggtgcag atgatgaaac tgggttcatc ccagttccaa agactcagag |
| 1801 | aactagagtt taagctgagg cagagtgccg ccaccctggc atgccccaca aacagatcac |
| 1861 | cagccagctt acacaggcat taactctcct caatgaggaa gaatcattca caactgagca |
| 1921 | agacattcat atgatcattt aaggaagtgt ttcccttatg tgttagcaag tataatcggc |
| 1981 | taactcctaa atcccaatga atagtcctag gctggacagc aatgggctgc aattaggcag |
| 2041 | ataaagacat cagtcccagt aaatgaatcc atagactcat ctagcaccaa ctaccattag |
| 2101 | cactatgtta ggagctgcaa ggccccaaag tagaagatgt gcataatgtc tgctcttgtg |
| 2161 | tagctcagga gacaattcca gcacagacac tacagttaac gctgaactgc agctgcaagt |
| 2221 | aatagcatga acagtcagaa aaatacctta tgagggggca gggctgaagc tgggccttga |
| 2281 | aggatggatg aaatttggat agagaatgag gaagacagag ggcctccaag tgagagaagc |
| 2341 | atgaaaaatg agcaggggcc tggatcagtg gggtgtattc agagcacctc tccagatgca |
| 2401 | ccatgcatgc tcacagtccc ttgcctatgt gtggcagagt gtcccagcca gatgtgtgcc |
| 2461 | ctcaccccat gtccatttac atgtccttca atgcccacct caaaaggtac ctcttctgta |
| 2521 | aagctttccc tggtatcagg aatcaaaatt aatcagggat cttttcacac tgctgttttt |
| 2581 | tcctctttgg tccttctatc actaaaactc atctcattca gccttagagc ataactaatt |
| 2641 | atttgttttc ctcactacat tgtacatgtg ggaattacag ataaacggaa gccggctggg |
| 2701 | gtggtggctc acgcctgtaa tcccaacact ttgggaggcc aaggcaggcg gatcacctga |
| 2761 | ggtcaggagt tcgagattag tctggccaac atggtgaaac cccatctcta ctaaaaatac |
| 2821 | gaaattagcc aggtgtggtg gcacacatct gtagtcccag ctactctgga ggctgagaca |
| 2881 | gcccaatcgc ttgaacccag gaagtggagg ttgcagtgag ctgagatcac accactgcac |
| 2941 | tccagcctgg gagagacaga gtgagactgg atctcgaaaa aaaaaaaaag atagaagcca |
| 3001 | ataagcatgg tgcaatcaaa ttctggcaag cattaaatat caggatgcag ctgggcacgg |
| 3061 | tggctcacgc ctgtaatccc agcactttgg gaggccaagg tgggcggatc acttgaggtc |
| 3121 | aggaatttga gaggatcctg gccagcatgg caaaacccca tctgtactta aaatacaaaa |
| 3181 | aaattagctg ggcgtggtgg tgcacacctg taatcccagc tacttgggag gctgaggtgg |
| 3241 | gagaattgct tgaacctggg aggtggaggt tgcagtgagc tgagatcctg ccactgcact |
| 3301 | ccaggctggg caacagagtg agaccatgtc tcaaaaaata aaaataaaat aaaataatat |
| 3361 | caggatgcat acatcagagg ctgttcctag tgtaaaggca ctttggaggg agaagacttt |
| 3421 | cagagttagg cagaccaact aagaggtcag ctgaagcacc taaccagttg taaggaggtg |
| 3481 | aaagacagca ccccaagaag agacgtgcag gaaggaggaa agaggcttgg tcataaagga |
| 3541 | tggaggaatt ccaaagtgac actgaacagg ctgcgtttat cctaaaataa aaccactcct |
| 3601 | cactctgtgg atgcgttgaa gactcattcc caaacatctt tattctctaa cttggcctct |
| 3661 | tcctcttcct aatatgctca ctcaagtaaa attactagtg tcctaatgcc cctatgcata |
| 3721 | ttgtcaaaaa taaaaatcag aagcaggtta gatctgttag gtcttccaga agagcaaacc |
| 3781 | tgggatccag ccagagccca ggaattctga aggtagcctt tggactcagg acaccctact |
| 3841 | cttgtctctc ctctcagttt ctctgctatg aatctcctga ttcatgaaca cgttatctgt |
| 3901 | tcacccttct ctctaggtct tagttcttag attttccttc tgtaaaatgc atgtgatctt |
| 3961 | attttcccct ccacaacttt ccagatgaac tagactgtga ccaagaggtc tataaaatca |
| 4021 | aagcatcatg gaagaggatc ttgtatcaga ccaaagtgtg ccagttttta aaaatgtgca |
| 4081 | tcaaaatgga agtctcagag acagagccct ctggtggaaa gttctagtag gttaggacag |
| 4141 | tcctgcctgc agacaccttg ggctttactg agggactcaa ctgagaaaat gaggaatgtt |
| 4201 | gcagctcatg attcttagaa gaagaaagtg aagcttgttt aaaatatgat ttaaaaaatc |
| 4261 | tgtagaacac tgtaaactac acaggctatg agggaatagc ctggttgggc cagcttggaa |
| 4321 | atcgggcaca ggcaggaagg ggcctgtctg gtttgggccg tgtccacaga gagcacttct |
| 4381 | taggtcctgc ctggagagaa ggaatggctg ggctatattt tcttccagac tcattatttt |
| 4441 | tcttctgttt gacttttctc tgaatttccc ttgatttgta taaattttct caataattag |
| 4501 | tgacagtgtc tactgattgt aaaatgaagc ttgaaggcca ggcgcagtgg ctcatgcctg |
| 4561 | taatcctaga attttgggag gccaaggtgg gtggatcaca aggagttcga gaccagcctg |
| 4621 | gccaaggttg tgaaaccgcg tctctactaa aaatacaaaa aaattagccg ggcatggtgg |
| 4681 | cacgtgcctg tagtcccagc tactcaggaa gctgaggcaa cagaatcact tgaacctggg |
| 4741 | aggtggaggt tgcagtgagc cgagatcacg ccactgcact ccagcctggg cgagagagtg |
| 4801 | agactccgtc tcaaaaaaaa aaaaaaaaaa aaaagtttga agaacaaaga caataagagg |
| 4861 | aaatataatg agtggtcata aatgtgggct ctgacagtag agtgcctggg tctgcatcct |
| 4921 | ggtttcttag tcatgtgacc ttaggcaagt tactttaacc tcgctgtacc tcaggttgtc |
| 4981 | catctgtaaa atggggataa taatagtggc taccttttaa ggttgatgtg gggattaaat |
| 5041 | gaggtgttgc tcatacagga atgtgcctgt gcatggcaaa gttcgggaaa ttttttataa |
| 5101 | gctgttctag gcctgaaatc ttcagaagat gctaatctaa attcatgaaa taagcttctt |
| 5161 | acaacagaaa tgctgctagt attatgcaaa attaatgttg tatatcaaac ttttaactct |
| 5221 | catccctcct tattcagata tattttgtta taagcaatgt ttgttcccct cgttattata |
| 5281 | ccacagtcta cttacctgat gctatatctg cctccccagt tagactgaga gaacagggga |
| 5341 | tatacctaaa taataataat aataataata ataataaata ataatggaga gctccttgaa |
| 5401 | gatagggagc ctgtaagaat cattgagggc ttattttgta taccaactgc taaactagat |
| 5461 | gcttcataca ttgttgtcaa tactcatgac agccttgtaa agtagaaatt aattcttcca |
| 5521 | gttaacacta aggctgacat atgaatacct tggcaaatct ggaaagctgg gaagacagta |
| 5581 | tttgaattca agacttcttg tcaccaaggg ccatgcactt gtactctgcc atgtggccct |
| 5641 | tttttacctc ctgtggattc tccctacctg gtacttggcc ttaggtgtac acacacctgg |
| 5701 | cactttgctt gacacataat aggtggacca caaatatcta ctaaatgaat atttgcatat |
| 5761 | agtaatattt taaggtactd aaagcagctc aaagtaaata tattaatata ttaattccat |
| 5821 | tgctatctgg ataaccactc aactttcctg ctgaaaatgc ccatttaatt aaagaaggtt |
| 5881 | ggatagagct ctctatatgc attttggaca ggcaggggtt tcaggtcata aacattctga |
| 5941 | tgagttaata taaaataaga gaaactgtaa atttccacta ctaaaaatca caaaaataac |
| 6001 | accaacaaaa gaagagataa gaatttgggg aattgtgctg aacaatttag tggttaaaaa |
| 6061 | aaacaactgt gcatgtttag acttaaataa gcccccatcc aagtgtgagg ggtccagtaa |
| 6121 | tttttcaaaa catatgaaag tgttaataca tttcgacaaa ggaccattaa aaaagtcctg |
| 6181 | aattctgact tgagggagga aagtaatgac taatacattc tctagagact tgcagacttt |
| 6241 | gggaattcat aaaggaatgg atgataatta ttaactgttg ctggctgatt gcccagacag |
| 6301 | ttctcaacag ccctgtacaa gtctctgggt ttgggatgga tcaattctga gactggaaaa |
| 6361 | tggccaaatc tttgcaaatg agaaatattt ttcttataag ttcttattgt aggcaaataa |
| 6421 | ttacatagat tattcatcag agaattttta aatgctcata atctcaactc tttcatttac |
| 6481 | aacttgtatt tccaatagtt tatgggtcat ctctgcatag atgtcagaag tcacctcaag |
| 6541 | tttagcgtgt ccaaaatcta actcacaggt ctgtttctga cctcccaact tgctttcctt |
| 6601 | gtgtttttcc tatgctaatg atccaccata atcaaaataa ttaacattta tccagtgcct |
| 6661 | actatgtact attccctgtc ctgttttaca tttactcatt taaagtccat aagaaacatt |
| 6721 | aaatctcatc tgccttctga agaagataca accatgctct cttttacaaa gtaggaaact |
| 6781 | gggtcacaga aaggtgaagt ctttaaggct gaatcacagt agctcatcct agtaaataga |
| 6841 | aaagccagga ttcaactcca ggggctgggt gcagaactgc tattcttcac tgcttcacca |
| 6901 | atcagcagct acccaaggca gaaaactttt tcatcattgg ctccttcatt ctccctgtca |
| 6961 | ccccagatcc cctctacatc tagtcagaga ataggtcctg tcaattccaa cttctctata |
| 7021 | tggctcctct caggcatgtg cccttaattg gcctaattct ctaatacacc ttccctctac |
| 7081 | atgctcactc cctcagatca ttgctttatc acgtgttacc tgggttgcta ttacataaag |
| 7141 | agcaatcttt ctaaaatgag gatattatca cttcacttcc acactaaaat gtttttcctg |
| 7201 | gggaaccaca ctccttagca atctgaccca tcagaccttc caggctgtct cctgcctgct |
| 7261 | ccctaaggct ccagccacac agaattatca tgggcccaca cacccaccaa atcctcccat |
| 7321 | gcctttgccc atgttgtctg ggatgccctt ctctcccttc tgtctacatc aagcatcaga |
| 7381 | ctgaatatcc ctcttgtgcg gccttctaaa acctcccgtc caaagcgaaa tatattgccc |
| 7441 | tgtatttata cttttacagc atttggcaca caagtacaga gtagtagctt tttatcacat |
| 7501 | tctctgataa ttatatagat atggtatttc ttagctctct ctccaactgg ctaataagtt |
| 7561 | gctttttgtc tgagtgccta attttgtgtt ttgtgtctga gtgcctcagt tcctcaaaaa |
| 7621 | aaggtttttt gattagttca ttattcattt gaacatggaa attatgctca ctagtggcaa |
| 7681 | atgccactaa ccgtattcca gaagctaggt gtcatgtttg caataagata tattatccct |
| 7741 | tatacaagtc accttttatt tcaggcattt gtaaatgccc attaataaag tatggttcat |
| 7801 | aaattttacc ttgtaagtgc ctaagaaatg agactacaag ctccatttca gcaggacaca |
| 7861 | ataaatatta ttttataatg catctaaaaa aaaaaaaaaa aaa |

SEQ ID NO: 60 Human CD84 Isoform 4 Amino Acid Sequence (NP_001171811.1)

| | |
|---|---|
| 1 | maqhhlwill lclqtcrlgk pkitqslmas vnstcnvtlt csvekeeknv tynwsplgee |
| 61 | gnvlgifqtp edqeltytct aqnpvsnnsd sisarqlcad iamgfrthht gllsvlamff |
| 121 | llvlilssvf lfrlfkrrqd aaskktiyty imasrntqpa esriydeilq skvlpskeep |
| 181 | vntvysevqf adkmgkastq dskppgtssy eivi |

SEQ ID NO: 61 Human CD84 Transcript Variant 5 cDNA Sequence
(NM_001330742.1; CDS: 80-1099)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctggccg gaagcagctg gaaaagactc agaaatcttc acagtgaatg ggattctggg |
| 181 | agagtcagtc actttccctg taaatatcca agaaccacgg caagttaaaa tcattgcttg |
| 241 | gacttctaaa acatctgttg cttatgtaac accaggagac tcagaaacag cacccgtagt |
| 301 | tactgtgacc cacagaaatt attatgaacg gatacatgcc ttaggtccga actacaatct |
| 361 | ggtcattagc gatctgagga tggaagacgc aggagactac aaagcagaca taaatacaca |
| 421 | ggctgatccc tacaccacca ccaagcgcta caacctgcaa atctatcgtc ggcttgggaa |
| 481 | accaaaaatt acacagagtt taatggcatc tgtgaacagc acctgtaatg tcacactgac |
| 541 | atgctctgta gagaaagaag aaaagaatgt gacatacaat tggagtcccc tgggagaaga |
| 601 | gggtaatgtc cttcaaatct tccagactcc tgaggaccaa gagctgactt acacgtgtac |
| 661 | agcccagaac cctgtcagca acaattctga ctccatctct gcccggcagc tctgtgcaga |
| 721 | catcgcaatg ggcttccgta ctcaccacac cgggttgctg agcgtgctgg ctatgttctt |
| 781 | tctgcttgtt ctcattctgt cttcagtgtt tttgttccgt ttgttcaaga gaagacaagg |
| 841 | ttcctgcttg aacaccttca ctaagaaccc ttatgctgcc tcaaagaaaa ccatatacac |
| 901 | atatatcatg gcttcaagga acacccagcc agcagagtcc agaatctatg atgaaatcct |
| 961 | gcagtccaag gtgcttccct ccaaggaaga gccagtgaac acagtttatt ccgaagtgca |
| 1021 | gtttgctgat aagatgggga aagccagcac acaggacagt aaacctcctg ggacttcaag |
| 1081 | ctatgaaatt gtgatctagg ctgctgggct gaattctccc tctggaaact gagttacaac |
| 1141 | caccaatact ggcaggttcc ctggatccag atcttctctg cccaactctt actgggagat |
| 1201 | tgcaaactgc cacatctcag cctgtaagca aagcaggaaa ccttctgctg ggcatagctt |
| 1261 | gtgcctaaat ggacaaatgg atgcataccc ttcctgaaat gactcccttc tgaatgaatg |
| 1321 | acaaagcagg ttacctagta tagttttccc aaacttcttc ccatcatagc acatgtagaa |
| 1381 | aataatattt ttatggcaca ctgggataaa caagcaagat tgctcacttc tggaagctgc |
| 1441 | atatgactag aggcctcttg tgactggagg taacaaccct gcccagtaac tgtgggagaa |
| 1501 | ggggatcaat attttgcaca cctgtaatag gccatggcac accagccaag atgctctgct |
| 1561 | cacagtcagt atgtgtgaag atccctggtg cgtggccttc accacgcatc ttgagcaaat |
| 1621 | taggaaaatg tacccttcgc ttgaggcaga tgcagccctt cccccgagtg catggcttgg |
| 1681 | agagcagaat gtgggctgca tataagcaca ctcatccctt tgtctgggaa tctttgtgca |
| 1741 | gggcataaca ggcttagtaa gtccaaacac agatgacagt gctgtgtggg tctctgtcag |
| 1801 | agttgtggct ctcagccatg tagacacact ctccaaatgg agtgttggaa aatgttcttt |
| 1861 | ctgcagggtc tagagactgc tgggacactt ttcttggagt gctacttcag aagccttata |
| 1921 | ggattttctt tctggccaag atttccttct gtatcactcc aagcagcctc agcagaagaa |
| 1981 | gcagccatgc ccagtattcc cactctccaa aaggaactga ccagcttata tttctcacac |
| 2041 | ttctggggaa ctgggtataa tccaaccatc aaaatagaag accttgcaag aagcagagtc |
| 2101 | attctccaga aggaacttgg gagatgatgg tgcagatgat gaaactgggt tcatcccagt |
| 2161 | tccaaagact cagagaacta gagtttaagc tgaggcagag tgccgccacc ctggcatgcc |
| 2221 | ccacaaacag atcaccagcc agcttacaca ggcattaact ctcctcaatg aggaagaatc |
| 2281 | attcacaact gagcaagaca ttcatatgat catttaagga agtgtttccc ttatgtgtta |
| 2341 | gcaagtataa tcggctaact cctaaatccc aatgaatagt cctaggctgg acagcaatgg |
| 2401 | gctgcaatta ggcagataaa gacatcagtc ccagtaaatg aatccataga ctcatctagc |
| 2461 | accaactacc attagcacta tgttaggagc tgcaaggccc caaagtagaa gatgtgcata |
| 2521 | atgtctgctc ttgtgtagct caggagacaa ttccagcaca gacactacag ttaacgctga |
| 2581 | actgcagctg caagtaatag catgaacagt cagaaaaata ccttatgagg gggcagggct |
| 2641 | gaagctgggc cttgaaggat ggatgaaatt tggatagaga atgaggaaga cagagggcct |
| 2701 | ccaagtgaga gaagcatgaa aaatgagcag gggcctggat cagtggggtg tattcagagc |
| 2761 | acctctccag atgcaccatg catgctcaca gtcccttgcc tatgtgtggc agagtgtccc |
| 2821 | agccagatgt gtgccctcac cccatgtcca tttacatgtc cttcaatgcc cacctcaaaa |
| 2881 | ggtacctctt ctgtaaagct ttccctggta tcaggaatca aaattaatca gggatctttt |
| 2941 | cacactgctg ttttttcctc tttggtcctt ctatcactaa aactcatctc attcagcctt |
| 3001 | acagcataac taattatttg ttttcctcac tacattgtac atgtgggaat tacagataaa |
| 3061 | cggaagccgg ctggggtggt ggctcacgcc tgtaatccca acactttggg aggccaaggc |
| 3121 | aggcggatca cctgaggtca ggagttcgag attagtctgg ccaacatggt gaaaccccat |
| 3181 | ctctactaaa aatacgaaat tagccaggtg tggtggcaca catctgtagt cccagctact |
| 3241 | ctggaggctg agacaggaga atcgcttgaa cccaggaagt ggaggttgca gtgagctgag |
| 3301 | atcacaccac tgcactccag cctgggagag acagagtgag actccatctc gaaaaaaaaa |
| 3361 | aaaagataga agccaataag catggtgcaa tcaaattctg gcaagcatta aatatcagga |
| 3421 | tgcagctggg cacggtggct cacgcctgta atcccagcac tttgggaggc caaggtgggc |
| 3481 | ggatcacttg aggtcaggaa tttgagagga tcctggccag catggcaaaa ccccatctgt |
| 3541 | acttaaaata caaaaaaatt agctgggcgt ggtggtgcac acctgtaatc ccagctactt |
| 3601 | gggaggctga ggtgggagaa ttgcttgaac ctgggaggtg gaggttgcag tgagctgaga |
| 3661 | tcctgccact gcactccagg ctgggcaaca gagtgagacc atgtctcaaa aaataaaaat |
| 3721 | aaaataaaat aatatcagga tgcatacatc agaggctgtt cctagtgtaa aggcactttg |
| 3781 | gagggagaag actttcagag ttaggcagac caactaagag gtcagctgaa gcacctaacc |
| 3841 | agttgtaagg aggtgaaaga cagcacccca agaagagacg tgcaggaagg aggaaagagg |
| 3901 | cttggtcata aaggatggag gaattccaaa gtgacactga acaggctgcg tttatcctaa |
| 3961 | aataaaacca ctcctcactc tgtggatgcg ttgaagactc attcccaaac atctttattc |
| 4021 | tctaacttgc cctcttcctc ttcctaatat gctcactcaa gtaaaattac tagtgtccta |
| 4081 | atgcccctat gcatattgtc aaaaataaaa atcagaagca ggttagatct gttaggtctt |
| 4141 | ccagaagagc aaacctggga tgaagccaga gcccaggaat tctgaaggta gcctttggac |
| 4201 | tcaggacacc ctactcttgt ctctcctctc agtttatctg ctatgaatct cctgattcat |
| 4261 | gaacacgtta tctttcacac cttctctcta ggtcttagtt cttagatttt ccttctgtaa |
| 4321 | aatgcatgtg atcttatttt cccctccaca actttccaga tgaactagac tgtgaccaag |
| 4381 | aggtctataa aatcaaagca tcatggaaca ggatcttgta tcagaccaaa gtgtgccagt |
| 4441 | ttttaaaaat gtgcatcaaa atggaagtct cagagacaga gccctctggt ggaaagttct |
| 4501 | agtaggttag gacagtcctg cctgcagaca ccttgggctt tactgaggga ctcaactgag |
| 4561 | aaaatgagga atgttgcagc tcatgattct tagaagaaga aagtgaagct tgtttaaaat |
| 4621 | atgatttaaa aaatctgtag aacactgtaa actacacagg ctatgaggga atagcctggt |
| 4681 | tgggccagct tggaaatcgg gcacaggcag gaaggggcct gtctggtttg ggccgtgtcc |
| 4741 | acagagagca cttcttaggt cctgcctgga gagaaggaat ggctgggcta tattttcttc |
| 4801 | cagactcatt atttttcttc tgtttgactt ttctctgaat ttcccttgat ttgtataaat |
| 4861 | tttctcaata attagtgaca gtgtctactg attgtaaaat gaagcttgaa ggccaggcgc |
| 4921 | agtggctcat gcctgtaatc ctagaatttt gggaggccaa ggtgggtgga tcacaaggag |
| 4981 | ttcgagacca gcctggccaa ggttgtgaaa ccgcgtctct actaaaaata caaaaaaatt |
| 5041 | agccgggcat ggtggcacgt gcctgtagtc ccagctactc aggaagctga ggcaacagaa |
| 5101 | tcacttgaac ctgggaggtg gaggttgcag tgagccgaga tcacgccact gcactccagc |
| 5161 | ctgggcgaga gagtgagact ccgtctcaaa aaaaaaaaaa aaaaaaaaag tttgaagaac |
| 5221 | aaagacaata agaggaaata taatgagtgg tcataaatgt gggctctgac agtagagtgc |
| 5281 | ctgggtctgc atcctggttt cttagtcatg tgaccttagg caagttactt taacctcgct |
| 5341 | gtacctcagg ttgtccatct gtaaaatggg gataataata gtgcctacct tttaaggttg |
| 5401 | atgtggggat taaatgaggt gttgctcata caggaatgtg cctgtgcatg gcaaagttcg |
| 5461 | ggaaattttt tataagctgt tctaggcctg aaatcttcag aagatgctaa tctaaattca |
| 5521 | tgaaataagc ttcttacaac agaaatgctg ctagtattat gcaaaattaa tgttgtatat |
| 5581 | caaactttta actctcatcc ctccttattc agatatattt tgttataagc aatgtttgtt |
| 5641 | cccctcgtta ttataccaca gtctacttac ctgatgctat atctgcctcc ccagttagac |
| 5701 | tgagagaaca ggggatatac ctaaataata ataataataa taataataat aaataataat |
| 5761 | ggagagctcc ttgaagatag ggagcctgta agaatcattg agggcttatt ttgtatacca |
| 5821 | actgctaaac tagatgcttc atacattgtt gtcaatactc atgacagcct tgtaaagtag |
| 5881 | aaattaattc ttccagttaa cactaaggct gacatatgaa taccttggca aatctggaaa |
| 5941 | gctgggaaga cagtatttga attcaagact tcttgtcacc aagggccatg cacttgtact |
| 6001 | ctgccatgtg gccctttttt acctcctgtg gattctccct acctggtact tggccttagg |
| 6061 | tgtacacaca cctggcactt tgcttgacac ataataggtg gaccacaaat atctactaaa |
| 6121 | tgaatatttg catatagtaa tattttaagg tactaaaagc agctcaaagt aaatatatta |
| 6181 | atatattaat tccattgcta tctggataac cactcaactt tcctgctgaa aatgcccatt |
| 6241 | taattaaaga aggttggata gagctctcta tatgcatttt ggacaggcag gggtttcagg |
| 6301 | tcataaacat tctgatgagt taatataaaa taagagaaac tgtaaatttc cactactaaa |
| 6361 | aatcacaaaa ataacagaaa caaaagaaga gataagaatt tggggaattg tgctgaacaa |
| 6421 | tttagtggtt aaaaaaaaca actgtgcatg tttagactta aataagcccc catccaagtg |
| 6481 | tgaggggtcc agtaattttt caaaacatat gaaagtgtta atacatttcg acaaaggacc |
| 6541 | attaaaaaag tcctgaattc tgacttgagg gaggaaagta atgactaata cattctctag |
| 6601 | agacttgcag actttgggaa ttcataaagg aatggatgat aattattaac tgttgctggc |
| 6661 | tgattgccca gacagttctc aacagccctg tacaagtctc tgggtttggg atggatcaat |
| 6721 | tctgagactg gaaaatggcc aaatctttgc aaatgagaaa tatttttctt ataagttctt |
| 6781 | attgtaggca aataattaca tagattattc atcagagaat ttttaaatgc tcataatctc |
| 6841 | aactctttca tttacaactt gtatttccaa tagtttatgg gtcatctctg catagatgtc |
| 6901 | agaagtcacc tcaagtttag cgtgtccaaa atctaactca caggtctgtt tctgacctcc |
| 6961 | caacttgctt tccttgtgtt tttcctatgc taatgatcca ccataatcaa aataattaac |
| 7021 | atttatccag tgcctactat gtactattcc ctgtcctgtt ttacatttac tcatttaaag |
| 7081 | tccataagaa acattaaatc tcatctgcct tctgaagaag atacaaccat gctctctttt |
| 7141 | acaaagtagg aaactgggtc acagaaaggt gaagtcttta aggctgaatc acagtagctc |
| 7201 | atcctagtaa atagaaaagc caggattcaa ctccaggggc tgggtgcaga actgctattc |
| 7261 | ttcactgctt caccaatcag cagctaccca aggcagaaaa ctttttcatc cttggctcct |
| 7321 | tcattctccc tgtcacccca gatcccctct acatctagtc agagaatagg tcctgtcaat |
| 7381 | tccaacttct ctatatggct cctctcaggc atgtgccctt aattggccta attctctaat |
| 7441 | acaccttccc tctacatgct cactccctca gatcattgct ttatcacgtg ttacctgggt |
| 7501 | tgctattaca taaagagcaa tctttctaaa atgaggatct tatcacttca cttccacact |
| 7561 | aaaatgtttt tcctggggaa ccacactcct tagcaatctg acccatcaga ccttccaggc |
| 7621 | tgtctcctgc ctgctcccta aggctccagc cacacagaat tatcatgggc ccacacaccc |
| 7681 | accaaatcct cccatgcctt tgcccatgtt gtctgggatg cccttctctc ccttctgtct |
| 7741 | acatcaagca tcagactgaa tatccctctt gtgcggcctt ctaaaacctc ccgtccaaag |
| 7801 | cgaaatatat tgccctctat ttatactttt acagcatttg gcacacaagt acagagtagt |
| 7861 | agctttttat cacattctct gataattata tagatatggt atttcttagc tctctctcca |
| 7921 | actggctaat aagttgcttt ttgtctgagt gcctaatttt gtgttttgtg tctgagtgcc |
| 7961 | tcagttcctc aaaaaaaggt tttttgatta gttcattatt catttgaaca tggaaattat |
| 8041 | gctcactagt ggcaaatgcc actaaccgta ttccagaagc taggtgtcat gtttgcaata |
| 8101 | agatatatta tcccttctac aagtcacctt ttatttcagg catttgtaaa tgcccattaa |
| 8161 | taaagtatgg ttcataaatt ttaccttgta agtgcctaag aaatgagact apaagctcca |
| 8221 | tttcagcagg acacaataaa tattatttta taatgcatct a |

SEQ ID NO: 62 Human CD84 Isoform 5 Amino Acid Sequence (NP_001317671.1)

| | |
|---|---|
| 1 | maqhhlwill lclqtwpeaa gkdseiftvn gilgesvtfp vniqeprqvk iiawtsktsv |
| 61 | ayvtpgdset apvvtvthrn yyerihalgp nynlvisdlr medagdykad intqadpytt |
| 121 | tkrynlqiyr rlgkpkitqs lmasvnstcn vtltcsveke eknvtynwsp lgeegnvlqi |
| 181 | fqtpedqelt ytctaqnpvs nnsdsisarq lcadiamgfr thhtgllsvl amffllvlil |
| 241 | ssvflfrlfk rrqgsclntf tknpyaaskk tiytyimasr ntqpaesriy deilqskvlp |
| 301 | skeepvntvy sevqfadkmg kastqdskpp gtssyeivi |

SEQ ID NO: 63 Mouse CD84 Transcript Variant 1 cDNA Sequence (NM_013489.3;
CDS: 180-1169)

| | |
|---|---|
| 1 | agtgcttgga gttcctctgt gactgaccac ttcttccttt tctgtctaat ggtgaacacc |
| 61 | tttctggacc agctctggac cagaatctga tttatgctct gctccggaaa caccacactg |
| 121 | aagtgaaagc agctaccaca ccagttattt ttcctcagaa gactggagtc tgactggaca |
| 181 | tggcccagcg ccatctgtgg atctggttcc tttgcctaca aacctggtct gaagcagcag |
| 241 | gaaaagatgc agacccggtg gtaatgaatg ggattcttgg ggagtcagtt actttcctct |
| 301 | taaatattca agaaccaaag aaaattgaca acattgcctg gacttctcaa tcatctgttg |
| 361 | cttttataaa accaggagtc aataaagctg aagttaccat aacccagggc acttataaag |
| 421 | gacgaataga aatcatagat cagaagtatg acctggtcat tagagacctg aggatggaag |
| 481 | atgcaggaac ttacaaagca gacatcaatg aagagaatga ggaaaccatc accaagatct |
| 541 | actaccttca tatctaccgt cgacttaaaa caccaaaaat tacacagagt ttgatatcat |
| 601 | ctttgaacaa tacctgtaat atcacactga catgctctgt ggaagaggaa gagaaggatg |
| 661 | tcacatatag ctggagtccc tttggagaga aaagcaatgt ccttcaaatc gtccactccc |
| 721 | ccatggacca aaaactgacc tacacatgta cagcccagaa ccctgtcagc aacagttctg |
| 781 | actctgtcac tgtccagcag ccatgtacag acactccaag cttccatcct cgccatgctg |
| 841 | tgttgccagg aggattggcc gtgctctttc tgcttattct cattccgatg ttggcatttc |
| 901 | tgttccgttt gtataagaga aggcgagaca ggattgtcct ggaagcagat gatgtctcaa |
| 961 | agaaaacagt atatgctgta gtttcaagaa atgctcaacc cacagagtcc agaatctatg |
| 1021 | atgaaatccc tcagtccaag atgctgtcct gtaagaaaga tccggtgacc accatttatt |
| 1081 | cctcagtgca gctttctgag aagatgaagg aaaccaacat gaaggacaga agtptgccta |
| 1141 | aggctttggg taatgaaatt gttgtctagg tgattctcta agaccacgaa ggacacaagg |
| 1201 | acaagtcatc tatgaggatt ccatcaacgg tttcagtctt ttggatataa cctgggccag |
| 1261 | ccaagggatt taggaatgaa gcaagctccg tgggtagagg tctgatcccc agtgtgtaat |
| 1321 | gttaggggcc atgtacagga ttgactctca ggcccacaga tctttaccca gagaaaccct |
| 1381 | gacctgctcc catgctgttt ttcctgggga aaggacccta gggcactcaa cctttatgca |
| 1441 | atcagacatg cctctcagag actgtctaac agcttccaga ctaatctctg tgcagtactt |
| 1501 | agtcttacaa ctctcacggg caacggcttc aagttccaat tttacgatgt gtctagcctg |
| 1561 | ggatgactgt ttagtttcta atgtggcgag aatgtatgtt accatgtagg aagcacagac |
| 1621 | tatggcaatc tataaatgat ttgtggcatg agactgatgt ccgaatttag gggaagggga |
| 1681 | atggtcttac ttaggcattt tatggaaatt gagtctctct ccccgagaag agggtgatga |
| 1741 | agcagcatcc acgtctgcct cttctccagt aacctgcttg ttatcgacaa tgtcgagccg |
| 1801 | atggtaatga actgagacaa atgcgcttga aagagatgaa tcaatttgag atttaacaaa |
| 1861 | tcgggtcaat ttctgaaatg cccaaggacc gaaggagatc aataatagga gtcccaatag |
| 1921 | gggccctaaa agggagggca acaaggttga aagtcagggg gaggttgaaa accagttttg |
| 1981 | gtagtctacc ttccccctag gccattgtaa atacttgtgt atgggtgtga ctcagctatc |
| 2041 | tatagttcta agtatccact ctggttcctc tttaggtctt aaacttcctt cttcctagtt |
| 2101 | gatggtaaat tcctgtgtaa ggcagtggcc tagcttttat tcaaagtgat agtgtaatgc |
| 2161 | cagaggctat tctgaatgtc actgaatagg caacactctc cctgaattct aagtccatgg |
| 2221 | tctgttcaag ggctttttag gacattggaa caccagtgaa ggcttagcta tgtcagaatt |
| 2281 | caatcttaaa atgcacttat aataagataa tattaaaaga gagcacatgg atctatacac |
| 2341 | cagactaact cggccataga atatgagtac aaaatgggca gtatgcagct gctgaactaa |
| 2401 | ggctgtggta gatacctttt caaagtttgc attcccagat ttttaaacca aggatcgttt |
| 2461 | cctaactcta ataggcagca aaacgtaagc aggtctctaa caaaaacata acagtagatt |
| 2521 | ccttatctaa attaggatct acacaattag ttaattgaca agaattacaa tgaatataaa |
| 2581 | aagacttgcc aagattggcg atcttaactc ttaaaattat ctaacaatga gcatataggt |
| 2641 | aaggtacaca aactttcata gctataagga gctgacctga aaggccaaaa acagtgtctc |
| 2701 | tgacaaaagc atcttgtaca ttctctgtac cagtcttttt gcctcatgag tcagcttttt |
| 2761 | tagttgtttt tattttaagt tggcaccagg ttggtactcc ttgctgcagc ccatggcgga |
| 2821 | gatacgaagt ttctttatct gtttgtaagt ggctgctctc tgatttctct tcttttgtat |
| 2881 | actcaacata gctttctggt caccactgtc agggcactca caggtcacag gtcagcctgt |
| 2941 | cacattggaa gctagcatgc tcttgtagca ttctgtggaa aaaacagaaa cattctccct |
| 3001 | tttccccata ttaagtatct gaacaggatc atggcaagtg ccaataagtg gatccttttt |
| 3061 | atctgtccta gacatcatta tatctagttt gttttttttt tgtaaataaa aatgtgattt |
| 3121 | tatgtgcaca gggatataat tcctaccttc tttgttttta aagaaggtat agtttttaaa |
| 3181 | gttttacaat accttgtctt tgagaattat aaaatatctc agtaacatgt gtaacattaa |
| 3241 | attgttaaca aaacatctct tggaggtttt gaaaataaaa attttgaagc |

SEQ ID NO: 64 Mouse CD84 Isoform 1 Amino Acid Sequence (NP_038517.1)

| | |
|---|---|
| 1 | maqrhlwiwf lclqtwseaa gkdadpvvmn gilgesvtfl lniqepkkid niawtsqssv |
| 61 | afikpgvnka evtitqgtyk grieiidqky dlvirdlrme dagtykadin eeneetitki |
| 121 | yylhiyrrlk tpkitqslis slnntcnitl tcsvekeekd vtyswepfge ksnvlqivhs |
| 181 | pmdqkltytc taqnpvsnss dsvtvqqpct dtpsfhprha vlpgglavlf llilipmlaf |
| 241 | lfrlykrrrd rivleaddvs kktvyavvsr naqptesriy deipqskmls ckkdpvttiy |
| 301 | ssvqlsekmk etnmkdrslp kalgneivv |

SEQ ID NO: 65 Mouse CD84 Transcript Variant 2 CDNA Sequence
(NM_001252472.1; CDS: 180-602)

| | |
|---|---|
| 1 | agtgcttgga gttcctctgt gactgaccac ttcttccttt tctgtctaat ggtgaacacc |
| 61 | tttctggacc agctctggac cagaatctga tttatgctct gctccggccc caccacactg |
| 121 | aagtgaaagc agctaccaca ccagttattt ttcctcagaa gactggagtc tgactggaca |
| 181 | tggcccagcg ccatctgtgg atctggttcc tttgcctaca aacctggtct gaagcagcag |
| 241 | gaaaagatgc agacccggtg gtaatgaatg ggattcttgg ggagtcagtt actttcctct |
| 301 | taaatattca agaaccaaag aaaattgaca acattgcctg gacttctcaa tcatctgttg |
| 361 | cttttataaa accaggagtc aataaagctg aagttaccat aacccagggc acttataaag |
| 421 | gacgaataga aatcatagat cagaagtatg acctggtcat tagagacctg aggatggaag |
| 481 | atgcaggaac ttacaaagca gacatcaatg aagagaatga ggaaaccatc accaagatct |
| 541 | actaccttca tatctaccgt aagttatggc agcacggggc cttggattta cttttgattt |
| 601 | gaagatttat gatctaaacc acacccatat ttctgataac agagtttcct aactcttctt |
| 661 | atccttataa ttacataagc acatcagtac ttataaaggt ctaactttac ttctggcctt |
| 721 | gacagacttt agctgtaatc tgttttgcag cagaatttgt cctctgttct ttgttttcct |
| 781 | ttcctataaa atgtcaataa tcatattaat caatttgtaa gcattattat gacattctag |
| 841 | taagaaacta tatgcagagt ggtctttata aggcttctgc ttttaagata attacagaat |
| 901 | gcataggcaa atgcaaagaa ctatgaaagg atgtgttctg tgcccttctc ttgccctctc |
| 961 | ctctgttata ctagatccaa actcttagtc tcatcccccg tcttatacag accttctggg |
| 1021 | gtagcctttc ttcattgtct ctcctgattc caaagagata aaataagcag atcctggcac |
| 1081 | acacctttga ttccagcact caggaggcaa agacagaagg atctctatga gttcaaggct |
| 1141 | agcttggtct tcagagaaag ttccagaaca gccaagacta acaaaaagaa accatttctc |
| 1201 | aaaccaacct cccatcccca tcctcacccc ccaaaaacta ctagaatgaa agaaaaaaaa |
| 1261 | ataccaagac acaaaatgaa cagtgtctga ggaactctta tttctgtaag tattaatact |
| 1321 | ttcagaaatg cagaacagtg ttcaaggtca aaaacagaaa attggaactt tcttggaatg |
| 1381 | tgccagcact tacttaggaa tggaatcact tatattccta tagaatttaa tcacatatat |
| 1441 | agcaccggac agcattaatc acatatatag caccggacat cataagcaag gtagtagtga |
| 1501 | agcaccatcc caacattttc ttgtctcagt tctgcagggc agagaactgt tagccctgac |
| 1561 | tccagctttt tctacctaac agttttgtgc aggtgacatc tcgtccttgc tgagaaaatg |
| 1621 | aaatgagatt tagttttcac cactatggct gtaccaatac ctaccccaac gggtggcaca |
| 1681 | cacacacaca cacacacaca cacacacaca cacacacaca cacacacgtc tctccgaaaa |
| 1741 | taggtaaata atgatacttt tcttgtgttt cggctatttt ggaatattca gagcctactg |
| 1801 | ttgtagaata gctgggataa aatggagaca tcctgcccag gctgttattg actgtgcttt |
| 1861 | tttgttggca tctaggcatc tgggactggg atgattataa atctaggtga tgatgtttgg |
| 1921 | atttgtcttt gttgggtggg tggcttgttc cttgttttct gtttcctctc tggattttca |
| 1981 | gagagtgtgg tagctctatg ttttagtagg acattttctt tggctctgac ttttatagcc |
| 2041 | actggaggtt ctcaataaaa gatgtttctg ggtattggga gctaacactt aggacctgtg |
| 2101 | ataggttagg agtatgaaag tgttcatagg agagagaaga agagtgttta gccaggatct |
| 2161 | gtttatttca tccccttgga atcagggaga cagtaaagtg aggtcaaccc acagggtctt |
| 2221 | ctctagatac tggggatgag actgggaagt tggatctaga ggaacagaag gaaacaggaa |
| 2281 | gatatacagg ctcctatctg cttctgggca ggaatgatct gggttagcag ggagtgcctg |
| 2341 | ctgaagatga gggctgggat aaaccaacaa gtggggagaa ggtgggtaga aagggaagat |
| 2401 | ctgtggaacc acaatagatg tgggattggg gatgtgcagg agtggggaca ttagaaggaa |
| 2461 | ggctgcagca ggtgttctgt tgcagagcta gggatgaagc tggagcttta gatttgtagg |
| 2521 | agaggaagcc ttctgttagc ttacatgttt cccaggcctg cttgggtggt gtgttcacaa |
| 2581 | ggaacactgg ttttggggac ttgtttactg gaatgaattg ggggaaggaa ggttggagta |
| 2641 | gaagatagag gtcctcaaag agaaaattaa taagtccaca aaatccaaac aaacagtgga |
| 2701 | aagaagtgaa taaactgtat aagacttgaa aatgaaaata aaatcaataa agaaaactaa |
| 2761 | aaccgagaga attctagaaa tgaadatttt aagaatttga acagaaatta cagaggtaaa |
| 2821 | cttcaccaac aaaatatgag agatggaaga gagaatatta ggcattgaag atacaataga |
| 2881 | gaaaatgaat atatcagtca aagaaaatgt taaaccaaaa aaaaaaagtc ctaacaaaaa |
| 2941 | atacaaaaaa tttaggacac taagaaaaga caaaacctaa gactaataga aatataggaa |
| 3001 | ggaaaagaat tctacctcaa aggcccagaa aatatttcaa caaaatcata gaagaaaaat |
| 3061 | tttctcacct aaagaagata gatgcctata aggtatatga agcatatata acaacaaata |
| 3121 | gatttaacaa gaaaataaac tctccttggc acataacagt caaatcacaa agcatacaga |
| 3181 | acaaagaaag aatattaaaa gctacaaggg gaaaaggcca agtagtatat aaatgcagac |
| 3241 | ctagaatgat acctgatttc tcagtgaaga ctctaaaggc caatagagtc tggacaaatg |
| 3301 | tgctataaac tctaagagac cccagaggtc atccctgatt actataccca ccaaaatatc |
| 3361 | caacatccta aatggaaaaa ataggatatt ccataataaa gccaaattta aacaatatct |
| 3421 | gtctacaaat ccatctatag aagatgacag ggcggaaaat tccaaccaaa agagtttaac |
| 3481 | tataccaaat aaaaacaaaa ggaataaaca atctcaaagc |

SEQ ID NO: 66 Mouse CD84 Isoform 2 Amino Acid Sequence (NP_001239401.1)

| | |
|---|---|
| 1 | maqrhlwiwf lclqtwseaa gkdadpvvmn gilgesvtfl lniqepkkid niawtsqssv |
| 61 | afikpgvnka evtitqgtyk grieiidqky dlvirdlrme dagtykadin eeneetitki |
| 121 | yylhiyrklw qhgaldllli |

SEQ ID NO: 67 Mouse CD84 Transcript Variant 3 cDNA Sequence
(NM_001289470.1; CDS: 180-1166)

| | |
|---|---|
| 1 | agtgcttgga gttcctctgt gactgaccac ttcttccttt tctgtctaat ggtgaacacc |
| 61 | tttctggacc agctctggac cagaatctga tttatgctct gctccggaaa caccacactg |
| 121 | aagtgaaagc agctaccaca ccagttattt ttcctcagaa gactggagtc tgactggaca |
| 181 | tggcccagcg ccatctgtgg atctggttcc tttgcctaca aacctggtct gaagcagcag |
| 241 | gaaaagatgc agacccggtg gtaatgaatg ggattcttgg ggagtcagtt actttcctct |
| 301 | taaatattca agaaccaaag aaaattgaca acattgcctg gacttctcaa tcatctgttg |
| 361 | cttttataaa accaggagtc aataaagctg aagttaccat aacccagggc acttataaag |
| 421 | gacgaataga aatcatagat cagaagtatg acctggtcat tagagacctg aggatggaag |
| 481 | atgcaggaac ttacaaagca gacatcaatg aagagaatga ggaaaccatc accaagatct |
| 541 | actaccttca tatctaccgt cgacttaaaa caccaaaaat tacacagagt ttgatatcat |
| 601 | ctttgaacaa tacctgtaat atcacactga catgctctgt ggaaaaggaa gaaaaggatg |
| 661 | tcacatatag ctggagtccc tttggagaga aaagcaatgt ccttcaaatc gtccactccc |
| 721 | ccatggacca aaaactgacc tacacatgta cagcccagaa ccctgtcagc aacagttctg |
| 781 | actctgtcac tgtccagcag ccatgtacag acactccaag cttccatcct cgccatgctg |
| 841 | tgttgccagg aggattggcc gtgctctttc tgcttattct cattccgatg ttggcatttc |
| 901 | tgttccgttt gtataagaga aggcgagaca ggattgtcct ggaagatgat gtctcaaaga |
| 961 | aaacagtata tgctgtagtt tcaagaaatg ctcaacccac agagtccaga atctatgatg |
| 1021 | aaatccctca gtccaagatg ctgtcctgta agaaagatcc ggtgaccacc atttattcct |
| 1081 | cagtgcagct ttctgagaag atgaaggaaa ccaacatgaa ggacagaagt ctgcctaagg |
| 1141 | ctttgggtaa tgaaattgtt gtctaggtga ttctctaaga ccacgaagga cacaaggaca |
| 1201 | agtcatctat gaggattgaa tcaacggttt cagtcttttg gatataacct gggccagcca |
| 1261 | agggatttag gaatgaagca agctccgtgg gtagaggtct gatccccagt gtgtaatgtt |
| 1321 | aggggccatg tacaggattg actctcaggc ccacagatct ttacccagag aaaccctgac |
| 1381 | ctgctcccat gctgtttttc ctggggaaag gaccctaggg cactcaacct ttatgcaatc |
| 1441 | agacatgcct ctcagagact gtctaacagc ttccagacta atctctgtgc agtacttagt |
| 1501 | cttacaactc tcacgggcaa cggcttcaag ttccaatttt acgatgtgtc tagcctggga |
| 1561 | tgactgttta gtttctaatg tggcgagaat gtatgttacc atgtaggaag cacagactat |
| 1621 | ggcaatctat aaatgatttg tggcatgaga ctgatgtccg aatttagggg aaggggaatg |
| 1681 | gtcttactta ggcattttat ggaaattgag tctctctccc cgagaagagg gtgatgaagc |
| 1741 | agcatccacg tctgcctctt ctccagtaac ctgcttgtta tcgacaatgt ccagccgatg |
| 1801 | gtaatgaact gaaacaaatg cgcttgaaag agatgaatca atttgagatt taacaaatcg |
| 1861 | ggtcaatttc tgaaatgccc aaggaccgaa ggagatcaat aataggagtc ccaatagggg |
| 1921 | ccctaaaagg gagggcaaca aggttgaaag tcagggggag gttgaaaacc agttttggta |
| 1981 | gtctaccttc cccctaggcc attgtaaata cttgtgtatg ggtgtaactc agctatctat |
| 2041 | agttctaagt atccactctg gttcctcttt aggtcttaaa cttccttctt cctagttgat |
| 2101 | ggtaaattcc tgtgtaaggc agtggcctag cttttattca aagtgatagt gtaatgccag |
| 2161 | aggctattct gaatgtcact gaataggcaa cactctccct gaattctaag tccatggtct |
| 2221 | gttcaagggc tttttaggac attggaacac cagtgaaggc ttagctatgt cagaattcaa |
| 2281 | tcttaaaatg cacttataat aagataatat taaaagagag cacatggatc tatacaccag |
| 2341 | actaactcgg gaatagaata tgagtacaaa atgggcagta tgcagctgct gaactaaggc |
| 2401 | tgtggtagat accttttcaa agtttgcatt cccagatttt taaaccaagg atcgttttct |
| 2461 | aactctaata ggcagcaaaa cgtaagcagg tctctaacaa aaacataaca gtagattcct |
| 2521 | tatctaaatt aggatctaca caattagtta attgacaaga attacaatga atataaaaag |
| 2581 | acttgccaag attggcgatc ttaactctta aaattatcta acaataagca tataggtaag |
| 2641 | gtacacaaac tttcatagct ataaggagct gacctccaag gccaaaaaca gtgtctctga |
| 2701 | caaaagcatc ttgtacattc tctgtaccag tctttttgcc tcatgagtca gcttttttag |
| 2761 | ttgtttttat tttaagttgg caccaggttg gtactccttg ctgcagccca tggcggagat |
| 2821 | acgaagtttc tttatctgtt tgtaagtggc tgctctctga tttctcttct tttgtatact |
| 2881 | caacatagct ttctggtcac cactgtcagg gcactcacag gtcacaggtc agcctgtcac |
| 2941 | attggaagct agcatgctct tgtagcattc tgtggaaaaa acagaaacat tctccctttt |
| 3001 | ccccatatta agtatctgaa caggatcatg gcaagtgcca ataagtggat cctttttatc |
| 3061 | tgtcctagac atcattatat ctagtttgtt ttttttttgt aaataaaaat gtgattttat |
| 3121 | gtgcacaggg atataattcc taccttcttt gtttttaaag aaggtatagt ttttaaagtt |
| 3181 | ttacaatacc ttgtctttga ccattataaa atatctcagt aacatgtgta acattaaatt |
| 3241 | gttaacaaaa catctcttgg aggttttgaa aataaaaatt ttccagc |

SEQ ID No: 68 Mouse CD84 Isoform 3 Amino Acid Sequence (NP_001276399.1)

| | |
|---|---|
| 1 | maqrhlwiwf lclqtwseaa gkdadpvvmn gilgesvtfl lniqepkkid niawtsqssv |
| 61 | afikpgvnka evtitqgtyk grieiidqky dlvirdirme dagtvkadin eeneetitki |
| 121 | yylhiyttlk tpkitqslis slnntcnitl tcsvekeekd vtyswspfge ksnvlqivhs |
| 181 | pmdqkltytc taqnpvsnss dsvtvqqpct dtpsfhprha vlpgglavlf llilipmlaf |
| 241 | lfrlykrrrd rivleddvsk ktvyavvsrn aqptesriyd eipqskmlsc kkdpvttiys |
| 301 | svqisekmke tnmkdrslpk algneivv |

SEQ ID NO: 69 Human IGSF6 Sequence (NM_005849.3; CDS: 69-794)

| | |
|---|---|
| 1 | ccttctgaaa aaagaaagcc aactttcctt tcaaatacac accccaaccc gccccggcat |
| 61 | acacagaaat ggggactgcg agcagaagca acatcgctcg ccatctgcaa accaatctca |
| 121 | ttctattttg tgtcggtgct gtgggcgcct gtactctctc tgtcacacaa ccgtggtacc |
| 181 | tagaagtgga ctacactcat gaggccgtca ccataaagtg taccttctcc gcaaccggat |
| 241 | gcccttctga gcaaccaaca tgcctgtggt ttcgctacgg tgctcaccag cctgagaacc |
| 301 | tgtgcttgga cgggtgcaaa agtgaggcag acaagttcac agtgagggag gccctcaaag |
| 361 | aaaaccaagt ttccctcact gtaaacagag tgacttcaaa tgacagtgca atttacatct |
| 421 | gtggaatagc attccccagt gtgccggaag cgagagctaa acagacagga ggagggacca |
| 481 | cactggtggt aagagaaatt aagctgctca gcaaggaact gcggagcttc ctgacagctc |
| 541 | ttgtatcact gctctctgtc tatgtgaccg gtgtgtgcgt ggccttcata ctcctctcca |
| 601 | aatcaaaatc caaccctcta agaaacaaag aaataaaaga agactcacaa aagaagaaga |
| 661 | gtgctcggcg tatttttcag gaaattgctc aagaactata ccataagaga catgtggaaa |
| 721 | caaatcagca atctgagaaa gataacaaca cttatgaaaa cagaagagta ctttccaact |
| 781 | atgaaaggcc atagaaacgt tttaattttc aatgaagtca ctgaaaatcc aactccagga |
| 841 | gctatggcag tgttaatgaa catatatcat caggtcttaa aaaaaaataa aggtaaactg |
| 901 | aaaagacaac tggctacaaa gaaggatgtc agaatgtaag gaaactataa ctaatagtca |
| 961 | ttaccaaaat actaaaaccc aacaaaatgc aactgaaaaa taccttccaa atttgccaag |
| 1021 | aaaaaaaatt ctattttaaa cttgaaaaaa aaaaaaaaaa aa |

SEQ ID NO: 70 Human IGSF6 Amino Acid Sequence (NP_005840.2)

| | |
|---|---|
| 1 | mgtasrsnia rhlqtnlilf cvgavgactl svtqpwylev dytheavtik ctfsatgcps |
| 61 | eqptclwfry gahqpenlcl dgckseadkf tvrealkenq vsltvnrvts ndsaiyicgi |
| 121 | afpsvpeara kqtgggttlv vreikllske lrsfltalvs llsvyvtgvc vafillsksk |
| 181 | snplrnkeik edsqkkksar rifqeiaqel yhkrhvetnq qsekdnntye nrrvlsnyer |
| 241 | p |

SEQ ID NO: 71 Mouse IGSF6 cDNA Sequence (NM_030691.1)

| | |
|---|---|
| 1 | ggagaaggcg gcacatgcag cagagatggg ccccgtgagt gcacgcagga gccgcctccg |
| 61 | gccagagatc agcctgatcc ttttccaagt cggtatggtg ggtgcctgca ctgtgtatgt |
| 121 | gctgcaacca ggttacctag aagtggacta cggttctgac gccgtcacca tggagtgtaa |
| 181 | cttttctaca gttggatgcc ctccagtgcc accaaagagc ttgtggtttc gctgtggtac |
| 241 | tcaccagcct gaagctctgt gcttggatgg atgcagaaat gaggcagaca agttcacagt |
| 301 | gaaagaaacc ctggacccgg accaagtctt cctcactgtt aacaggctgt ctccaaatga |
| 361 | cagtgcaatt tacatctgtg gaatagcatt tcccaatgaa ctgtcagcaa gcgctaaaca |
| 421 | cgttggaaag gggactacac tggtggtaag agaaagactt ttcagcaagg aggtgcgcag |
| 481 | tttcctgata gtgctcttag ctctgctctc tgtctacatc accggtgtgt gtgtgacctt |
| 541 | catagtcctc ttcaaatcaa aatctaacgg tccaagaagc agagaaacca aaggctcaaa |
| 601 | aaagaagagt gctcggcgta tctttcagga aattgctcaa gaattatacc ataagagata |
| 661 | tgtggaaaca agtcatctac ctgagcaaga gggcactgat gaaaacagaa aagcactccc |
| 721 | caaccctgga agagcataga tgtgcttgct ttttacttaa gccactgaca gtgcaactcc |
| 781 | agaatctacg gcaatgtgaa tggacataca gcaatcaaga caacatcaaa gagagctggg |
| 841 | gtatagctca gctggcagag tgcttgccta gtaggcacaa agccctagct ttgatcccca |
| 901 | gcaccacata aactcatcaa agtgaaacaa gcctgtattc ccaacattgt gaagtataaa |
| 961 | gagtcaccag ttcaaggtca tccctgagta taggattaac ctccagtcag agacacatca |
| 1021 | tcttgtctca aaagcaaccg tgaccaccaa aagaaaagga caggacaagt gggaaaacag |
| 1081 | ccggcctcgc cagacggcag agcataagta gctgtcacta attgtagcta cagaatatga |
| 1141 | aaacctcaag aaaactcaac tggaggacct tttttctaat tttccaagaa tagtctaaaa |
| 1201 | agccccactt tgaagaaaaa acttcatctt aacagttttt aaaaactgtt accatgttta |
| 1261 | tgttgtcagt ctacccaaca tactagatgt gtgataggca ttaactgaga gaaggcttca |
| 1321 | agttaaacca cagatctcag ttctgagggg aaataaatac tttcctgagt tgtaaaaatg |
| 1381 | atgaaacaat tagaatcaag tgagaagggc aaaaggagta aggagaagag caatttctga |
| 1441 | gtaagagaaa ctcattgtga acagtatctt ggaacaaaag tgatttcttc tgatactgta |
| 1501 | acggagcagt gggcagtgaa cattctccag ctgaggtata ggaaacaact tgggttgtac |
| 1561 | caccaacaaa acaatactac aagagaccag aggacgacta taaacaggaa acccaaagcc |
| 1621 | tatcagaaat gcctcaggaa tgcagacaac tgactctaga tgtcagtgtg gtaccaaaga |
| 1681 | actgcagccc tagtgagctt gaaaggaggg tcggatacaa caagggcctc actatctcac |
| 1741 | taaggtgacc tgagccaggc atgctggcac acacctttaa tcctaacact aaggaggcag |
| 1801 | aggcaggtga atttctgagt tcaaggccag cctgatctat agatcgagtt ccaggacagc |
| 1861 | cagggctgtt aacagaaaaa cactgtctca gaaaaaaagg gagtgggggc ttgacatgga |
| 1921 | ttgttctttg aatataagta ccaacaagga cacactgctc actaacttga tcacaggtct |
| 1981 | agtacagttg cttctaaaca ggtactaaat ataaatggca cacactctta aacatcacac |
| 2041 | actgtgatac acacatacac acacacacac acacacacac acaggtttga aatttacata |
| 2101 | cacaaaagga ataaaataat ggcatacaca gta |

SEQ ID NO: 72 Mouse IGSF6 Amino Acid Sequence (NP_109616.1)

| | |
|---|---|
| 1 | mgpvsarrsr lrpeislilf qvgmvgactv yvlqpgylev dygsdavtme cnfstvgcpp |
| 61 | vppkslwfrc gthqpealcl dgcrneadkf tvketldpdq vfltvnrlsp ndsaiyicgi |
| 121 | afpnelspsa khvgkgttlv vrerlfskev rsflivllal lsvyitgvcv tfivlfksks |
| 181 | ngprsretkg skkksarrif geiaqelyhk ryvetshlpe qegtdenrka lpnpgra |

SEQ ID NO: 73 Human CD48 Transcript Variant 1 cDNA Sequence (NM_001778.3;
CDS: 89-820)

| | |
|---|---|
| 1 | gtttggtaag ttccgttttt agccccggcc tttttctagc caggctctca actgtctcct |
| 61 | gcgttgctgg gaagttctgg aaggaagcat gtgctccaga ggttgggatt cgtgtctggc |
| 121 | tctggaattg ctactgctgc ctctgtcact cctggtgacc agcattcaag gtcacttggt |
| 181 | acatatgacc gtggtctcgg gcagcaacgt gactctgaac atctctgaga gcctgcctga |
| 241 | gaactacaaa caactaacct ggttttatac tttcgaccag aagattgtag aatgggattc |
| 301 | cagaaaatct aagtactttg aatccaaatt taaaggcagg gtcagacttg atcctcagag |
| 361 | tggcgcactg tacatctcta aggtccagaa agaggacaac agcacctaca tcatgagggt |
| 421 | gttgaaaaag actgggaatg agcaagaatg gaagatcaag ctgcaagtgc ttgaccctgt |
| 481 | acccaagcct gtcatcaaaa ttgagaagat agaagacatg gatgacaact gttatctgaa |
| 541 | actgtcatgt gtgatacctg gcgagtctgt aaactacacc tggtatgggg acaaaaggcc |
| 601 | cttcccaaag gagctccaga acagtgtgct tgaaaccacc cttatgccac ataattactc |
| 661 | caggtgttat acttgccaag tcagcaattc tgtgagcagc aagaatggca cggtctgcct |
| 721 | cagtccaccc tgtaccctgg cccggtcctt tggagtagaa tggattgcaa gttggctagt |
| 781 | ggtcacggtg cccaccattc ttggcctgtt acttacctga gatgagctct tttaactcaa |
| 841 | gcgaaacttc aaggccagaa gatcttgcct gttggtgatc atgctcctca ccaggacaga |
| 901 | gactgtatag gctgaccaga agcatgctgc tgaattatca acgaggattt tcaagttaac |
| 961 | ttttaaatac tggttattat ttaattttat atccctttgt tgttttctag tacacagaga |
| 1021 | tatagagata cacatgcttt tttcccaccc aaaattgtga caacattatg tgaatgtttt |
| 1081 | attatttttt aaaataaaca tttgatataa ttgtcaatta actgaaaaaa aaaaaaaaaa |
| 1141 | aaaaaaaaaa aaaaa |

SEQ ID NO: 74 Human CD48 Isoform 1 Amino Acid Sequence (NP_001769.2)

| | |
|---|---|
| 1 | mcsrgwdscl alellllpls llvtsiqghl vhmtvvsgsn vtlniseslp enykqltwfy |
| 61 | tfdqkivewd srkskyfesk fkgrvrldpq sgalyiskvq kednstyimr vlkktgneqe |
| 121 | wkiklqvldp vpkpvikiek iedmddncyl klscvipges vnytwygdkr pfpkelqnsv |
| 181 | lettlmphny srcytcqvsn sysskhgtvc lsppctlars fgvewiaswi vvtvptilgl |
| 241 | llt |

SEQ ID NO: 75 Human CD48 Transcript Variant 2 cDNA Sequence
(NM_001256030.1; CDS: 89-847)

| | |
|---|---|
| 1 | gtttggtaag ttccgttttt agccccggcc tttttctagc caggctctca actgtctcct |
| 61 | gcgttgctgg gaagttctgg aaggaagcat gtgctccaga ggttgggatt cgtgtctggc |
| 121 | tctggaattg ctactgctgc ctctgtcact cctggtgacc agcattcaag gtcacttggt |
| 181 | acatatgacc gtggtctccg gcagcaacgt gactctgaac atctctgaga gcctgcctga |
| 241 | gaactacaaa caactaacct ggttttatac tttcgaccag aagattgtag aatgggattc |
| 301 | cagaaaatct aagtactttg aatccaaatt taaaggcagg gtcagacttg atcctcagag |
| 361 | tggcgcactg tacatctcta aggtccagaa agaggacaac agcacctaca tcatgagggt |
| 421 | gttgaaaaag actgggaatg agcaagaatg gaagatcaag ctgcaagtgc ttgaccctgt |
| 481 | acccaagcct gtcatcaaaa ttgagaagat agaagacatg gatgacaact gttatctgaa |
| 541 | actgtcatgt gtgatacctg gcgagtctgt aaactacacc tggtatgggg acaaaaggcc |
| 601 | cttcccaaag gagctccaga acagtgtgct tgaaaccacc cttatgccac ataattactc |
| 661 | caggtgttat acttgccaag tcagcaattc tgtgagcagc aagaatggca cggtctgcct |
| 721 | cagtccaccc tgtaccctgg gtaagaagga tccctgggag ctgagggggg cacagggtaa |
| 781 | ctggagttgt tttgaacaaa gaaaggctgg gggtcctatt cagcctcctt gcacagtgtg |
| 841 | gtggtgaatc cctaaggtgt ctgggagagc tgggagacgt gggttctgcc accagctcta |
| 901 | ccaccacctc ccagccagct tacctcaact tcgtgggggc tcagtgttct cacctgcaaa |
| 961 | ggacgtttgg gagagatctc tgatactcct cttccctctc ccgctctaac aaagcatagt |
| 1021 | cctaacatct gaggccaggg tcatcataga gtagactgaa acatcagggt gagcagggag |
| 1081 | aaggaagggc aagtgggcga gcagctgtct agaggggctt cattagacag ccgaagtcag |
| 1141 | ccaaggaaag agggaccgag gtcattagac cgccaaagtc agccagggaa agagggactg |
| 1201 | aggagacggg cctgagagag gccgtcgagg aggcgtgaga gcctgagcct caggcgaagc |
| 1261 | ttctcctccc cagcctgatg ttcctagatg aacttaggaa gccagattcc cctgtctcct |
| 1321 | gggaggatcc actcatgagt gtcacacctg gctctagatc aggcctacac tggtgctagc |
| 1381 | atgggacagc taaggccatg ggttttagag tcagtcatac ctggggtcac ttctaggact |
| 1441 | gtcacttact agctaaacaa gttacttagc ttccccaagt catgttattc ctaaataaag |
| 1501 | gacaaaataa cagttcctat aaaaaaaaaa aaaaaaa |

SEQ ID NO: 76 Human CD48 Isoform 2 Amino Acid Sequence (NP_001242959.1)

| | |
|---|---|
| 1 | mcsrgwdscl alellllpls llvtsiqghl vhmtvvsgsn vtlniseslp enykqltwfy |
| 61 | tfdqkivewd srkskyfesk fkgrvrldpq sgalyiskvq kednstyimr vlkktgneqe |
| 121 | wkiklqvldp vpkpvikiek iedmddncyl klscvipges vnytwygdkr pfpkelqnsv |
| 181 | lettlmphny srcytcqvsn svsskngtvc lsppctlgkk dpwelrgaqg nwscfeqrka |
| 241 | ggpiqppctv ww |

SEQ ID NO: 77 Mouse CD48 Transcript Variant 1 cDNA Sequence (NM_007649.5;
CDS: 103-825)

| | |
|---|---|
| 1 | atacgacttc cggttttggg ttttgcttcc tgattgaagg gcaggcgccc tgacttctct |
| 61 | tacagttgtc tccagtgttc tgggccagct tatctaagta ttatgtgctt cataaaacag |
| 121 | ggatggtgtc tggtcctgga actgctactg ctgcccttgg gaactggatt tcaaggtcat |
| 181 | tcaataccag atataaatgc caccaccggc agcaatgtaa ccctgaaaat ccataaggac |
| 241 | ccacttggac catataaacg tatcacctgg cttcatacta aaaatcagaa gattttagag |
| 301 | tacaactata atagtacaaa gacaatcttc gagtctgaat ttaaaggcag ggtttatctt |
| 361 | gaagaaaaca atggtgcact tcatatctct aatgtccgga aagaggacaa aggtacctac |
| 421 | tacatgagag tgctgcgtga aactgagaac gagttgaaga taaccctgga agtatttgat |
| 481 | cctgtgccca agcattccat agaaatcaat aagactgaag cgtcgactga ttcctgtcac |
| 541 | ctgaggctat cgtgtgaggt aaaggaccag catgttgact atacttggta tgagagctcg |
| 601 | ggacctttcc ccaaaaagag tccaggatat gtgctcgatc tcatcgtcac accacagaac |
| 661 | aagtctacat tttacacctg ccaagtcagc aatcctgtaa gcagcaagaa cgacacagtg |
| 721 | tacttcactc taccttgtga tctagccaga tcttctggag tatgttggac tgcaacttgg |
| 781 | ctagtggtca caacactcat cattcacagg atcctgttaa cctgacaaga actcttctca |
| 841 | cccaagaagg caacttggaa gcacagagtc ttgccttcat ccctagcagt gttcctagcc |
| 901 | agcgaagcaa ctctggctct attggacaaa ggaaaatgtg ttactgaacg tctgcgagag |
| 961 | tttgcatgca tgctctatga aacaagcaca ggaccttgta cagtgctcca ccactgacct |
| 1021 | gtgtgcccag tcctttacaa agatttcaaa tcaacctttt aaaaactgtg cataatatct |
| 1081 | aattttatat accatagttg tttcccaaca tatattaaag ataaatgcat tctttttacc |
| 1141 | aaaatgtgac tatattattt tcatgttttc atatctcttt ttaaaataaa ttcttttaaa |
| 1201 | aaact |

SEQ ID NO: 78 Mouse CD48 Isoform 1 Amino Acid Sequence (NP_031675.1)

| | |
|---|---|
| 1 | mcfikqgwcl vlellllplg tgfqghsipd inattgsnvt lkihkdplgp ykritwlhtk |
| 61 | nqkileynyn stktifesef kgrvyleenn galhisnvrk edkgtyymrv lretenelki |
| 121 | tlevfdpvpk psieinktea stdschlrls cevkdqhvdy twyessgpfp kkspgyvldl |
| 181 | ivtpqnkstf ytcqvsnpvs skndtvyftl pcdlarssgv cwtatwlvvt tliihrillt |

SEQ ID NO: 79 Mouse CD48 Transcript Variant 2 cDNA Sequence
(NM_001360767.1; CDS: 103-558)

| | |
|---|---|
| 1 | atacgacttc cggttttggg ttttgcttcc tgattgaagg gcaggcgccc tgacttctct |
| 61 | tacagttgtc tccagtgttc tggggaagct tctctaagta ttatgtgctt cataaaacag |
| 121 | ggatggtgtc tggtcctgga actgctactg ctgccattgg gaactggatt tcaaggtcat |
| 181 | tcaataccag atataaatgc caccaccggc agcaatgtaa ccctgaaaat ccataaggac |
| 241 | ccacttggac catataaacg tatcacctgg cttcatacta aaaatcagaa gattttagag |
| 301 | tacaactata atagtacaaa gacaatcttc gagtctgaat ttaaaggcag ggtttatctt |
| 361 | gaagaaaaca atggtgcact tcatatctct aatgtccgga aagaggacaa aggtacctac |
| 421 | tacatgagag tgctgcgtga aactgagaac gagttgaaga taaccctgga agtatttgcc |
| 481 | agatcttctg gagtatgttg gactgcaact tggctagtgg tcacaacact catcattcac |
| 541 | aggatcctgt taacctgaca agaactcttc tcacccaaga aggcaacttg gaagcacaga |
| 601 | gtcttgcctt catccctagc agtgttccta gccagcgaag caactctggc tctattggac |
| 661 | aaaggaaaat gtgttactga acgtctgcga gagtttgcat gcatgctcta tgaaacaagc |
| 721 | acaggacctt gtacagtgct ccaccactga cctgtgtgcc cagtccttta caaagatttc |
| 781 | aaatcaacct tttaaaaact gtgcataata tctaatttta tataccctag ttgtttccca |
| 841 | acatatatta aagataaatg cattcttttt accaaaatgt gactatatta ttttcatgtt |
| 901 | ttcatatctc tttttaaaat aaattctttt aaaaaact |

SEQ ID NO: 80 Mouse CD48 Isoform 2 Amino Acid Sequence (NP_001347696.1)

| | |
|---|---|
| 1 | mcfikqgwcl vlellllplg tgfqghsipd inattgsnvt lkihkdplgp ykritwlhtk |
| 61 | nqkileynyn stktifesef kgrvyleenn galhisnvrk edkgtyymrv lretenelki |
| 121 | tlevfarssg vcwtatwlvv ttliihrill t |

SEQ ID NO: 81 Human CD33 Transcript Variant 1 cDNA Sequence (NM_001772.3;
CDS: 41-4135)

| | |
|---|---|
| 1 | tctgctcaca caggaagccc tggaagctgc ttcctcagac atgccgctgc tgctactgct |
| 61 | gcccctgctg tgggcagggg ccctggctat ggatccaaat ttctggctgc aagtgcagga |
| 121 | gtcagtgacg gtacaggagg gtttgtgcgt cctcgtgccc tgcactttct tccatcccat |
| 181 | accctactac gacaagaact ccccagttca tggttactgg ttccgggaag gagccattat |
| 241 | atccagggac tctccagtgg ccacaaacaa gctagatcaa gaagtacagg aggagactca |
| 301 | gggcagattc cgcctccttg gggatcccag taggaacaac tgctccctga gcatcgtaga |
| 361 | cgccaggagg agggataatg gttcatactt ctttcggatg gagagaggaa gtaccaaata |
| 421 | cagttacaaa tctccccagc tctctgtgca tgtgacagac ttgacccaca ggcccaaaat |
| 481 | cctcatccct ggcactctag aacccggcca ctccaaaaac ctgacctgct ctgtgtcctg |
| 541 | ggcctgtgag cagggaacac ccccgatctt ctcctggttg tcagctgccc ccacctccct |
| 601 | gggccccagg actactcact cctcggtgct cataatcacc ccacggcccc aggaccacgg |
| 661 | caccaacctg acctgtcagg tgaagttcgc tggagctggt gtgactacgg agagaaccat |
| 721 | ccagctcaac gtcacctatg ttccacagaa cccaacaact ggtatctttc caggagatgg |
| 781 | ctcagggaaa caagagacca gagcaggagt ggttcatggg gccattggag gagctggtgt |
| 841 | tacagccctg ctcgctcttt gtctctgcct catcttcttc atagtgaaga cccacaggag |
| 901 | gaaagcagcc aggacagcag tgggcaggaa tgacacccac cctaccacag ggtcagcctc |
| 961 | cccgaaacac cagaagaagt ccaagttaca tggccccact gaaacctcaa gctgttcagg |
| 1021 | tgccgcccct actgtggaga tggatgagga gctgcattat gcttccctca actttcatgg |
| 1081 | gatgaatcct tccaaggaca cctccaccga atactcagag gtcaggaccc agtgaggaac |
| 1141 | ccacaagagc atcaggctca gctagaagat ccacatcctc tacaggtcgg ggaccaaagg |
| 1201 | ctgattcttg gagatttaac accccacagg caatgggttt atagacatta tgtgagtttc |
| 1261 | ctgctatatt aacatcatct tagactttgc aagcagagag tcgtggaatc aaatctgtgc |
| 1321 | tctttcattt gctaagtgta tgatgtcaca caagctcctt aaccttccat gtctccattt |
| 1381 | tcttctctgt gaagtaggta taagaagtcc tatctcatag ggatgctgtg agcattaaat |
| 1441 | aaaggtacac atggaaaaca ccagtc |

SEQ ID NO: 82 Human CD33 Isoform 1 Amino Acid Sequence (NP_001763.3)

| | |
|---|---|
| 1 | mplllllpll wagalamdpn fwlqvqesvt vqeglcvlvp ctffhpipyy dknspvhgyw |
| 61 | fregaiisrd spvatnkldq evqeetqgrf rllgdpsrnn cslsivdarr rdngsyffrm |
| 121 | ergstkysyk spqlsvhvtd lthrpkilip gtlepghskn ltcsvswace qgtppifswl |
| 181 | saaptslgpr tthssvliit prpqdhgtnl tcqvkfagag vttertiqln vtyvpqnptt |
| 241 | gifpgdgsgk qetragvvhg aiggagvtal lalclcliff ivkthrrkaa rtavgrndth |
| 301 | pttgsaspkh qkksklhgpt etsscsgaap tvemdeelhy aslnfhgmnp skdtstevse |
| 361 | vrtq |

SEQ ID NO: 83 Human CD33 Transcript Variant 2 cDNA Sequence
(NM_001082618.1; CDS: 41-754)

| | |
|---|---|
| 1 | tctgctcaca caggaagccc tggaagctgc ttcctcagac atgccgctgc tgctactgct |
| 61 | gcccctgctg tgggcagact tgacccacag gcccaaaatc ctcatccctg gcactctaga |
| 121 | acccggccac tccaaaaacc tgacctgctc tgtgtcctgg gcctgtgagc agggaacacc |
| 181 | cccgatcttc tcctggttgt cagctgcccc cacctccctg ggccccagga ctactcactc |
| 241 | ctcggtgctc ataatcaccc cacggcccca ggaccacggc accaacctga cctgtcaggt |
| 301 | gaagttcgct ggagctggtg tgactacgga gagaaccatc cagctcaacg tcacctatgt |
| 361 | tccacagaac ccaacaactg gtatctttcc aggagatggc tcagggaaac aagagaccag |
| 421 | agcaggagtg gttcatgggg ccattggagg agctggtgtt acagccctgc tcgctctttg |
| 481 | tctctgcctc atcttcttca tagtgaagac ccacaggagg aaagcagcca ggacagcagt |
| 541 | gggcagccat gacacccacc ctaccacagg gtcagcctcc ccgaaacacc agaagaagtc |
| 601 | caagttacat ggccccactg aaacctcaag ctgttcaggt gccgccccta ctgtggagat |
| 661 | ggatgaggag ctgcattatg cttccctcaa ctttcatggg atgaatcctt ccaaggacac |
| 721 | ctccaccgaa tactcagagg tcaggaccca gtgaggaacc cacaagagca tcaggctcag |
| 781 | ctagaagatc cacatcctct acaggtcggg gaccaaaggc tgattcttgg agatttaaca |
| 841 | ccccacaggc aatgggttta tagacattat gtgagtttcc tgctatatta acatcatctt |
| 901 | agactttgca agcagagagt cgtggaatca aatctgtgct ctttcatttg ctaagtgtat |
| 961 | gatgtcacac aagctcctta accttccatg tctccatttt cttctctgtg aagtaggtat |
| 1021 | aagaagtcct atctcatagg gatgctgtga gcattaaata aaggtacaca tggaaaacac |
| 1081 | cagtc |

SEQ ID NO. 84 Human CD33 Isoform 2 Amino Acid Sequence (NP_001076087.1)

| | |
|---|---|
| 1 | mplllllpll wadlthrpki lipgtlepgh shnltcsvsw aceqgtppif swlsaaptsl |
| 61 | gprtthssvl iitprpqdhg tnltcqvkfa gagvtterti qlnvtyvpqn pttgifpgdg |
| 121 | sgkqetragv vhgaiggagv tallalclcl iffivkthrr kaartavgrn dthpttgsas |
| 181 | pkhqkksklh gptetsscsg aaptvemdee lhyaslnfhg mnpskdtste ysevrtq |

SEQ ID NO: 85 Human CD33 Transcript Variant 3 cDNA Sequence
(NM_001177608.1; CDS: 41-973)

| | |
|---|---|
| 1 | tatgctcaca caggaagccc tggaagctgc ttcctcagac atgccgctgc tgctactgct |
| 61 | gcccctgctg tgggcagggg ccctggctat ggatccaaat ttctggctgc aagtgcagga |
| 121 | gtcagtgacg gtacaggagg gtttgtgcgt cctcgtgccc tgcactttct tccatcccat |
| 181 | accctactac gacaagaact ccccagttca tggttactgg ttccgggaag gagccattat |
| 241 | atccagggac tctccagtgg ccacaaacaa gctagatcaa gaagtacagg aggagactca |
| 301 | gggcagattc cgcctccttg gggatcccag taggaacaac tgctccctga gcatcgtaga |
| 361 | cgccaggagg agggataatg gttcatactt ctttcggatg gagagaggaa gtaccaaata |
| 421 | cagttacaaa tctccccagc tctctgtgca tgtgacagac ttgacccaca ggcccaaaat |
| 481 | cctcatccct ggcactctag aacccggcca ctccaaaaac ctgacctgct ctgtgtcctg |
| 541 | ggcctgtgag cagggaacac ccccgatctt ctcctggttg tcagctgccc ccacctccct |
| 601 | gggccccagg actactcact cctcggtgct cataatcacc ccacggcccc aggaccacgg |
| 661 | caccaacctg acctgtcagg tgaagttcgc tggagctggt gtgactacgg agagaaccat |
| 721 | ccagctcaac gtcacctatg ttccacagaa cccaacaact ggtatctttc caggagatgg |
| 781 | ctcagggaaa caagagacca gagcaggagt ggttcatggg gccattggag gagctggtgt |
| 841 | tacagccctg ctcgctattt gtctctgcct catcttcttc atagtgaaga cccacaggag |
| 901 | gaaagcagcc aggacagcag tgggcaggaa tgacacccac cctaccacag ggtcagcctc |
| 961 | cccggtacgt tgaggccaac agatcaggag atgatggcca ttgaaaagat agtttcttgg |
| 1021 | ccgggcacag tgtttcacac ctgcaatccc agcacctttg gaggccaagg cgggcggatc |
| 1081 | acgaggtcag gagattgaga ctatcctg |

SEQ ID NO: 86 Human CD33 Isoform 3 Amino Acid Sequence (NP_001171079.1)

| | |
|---|---|
| 1 | mplllllpll wagalamdpn fwlqvqesvt vqeglcvlvp ctffhpipyy dknspvhgyw |
| 61 | fregaiisrd spvatnkldg evqeetqgrf rllgdpsrnn cslsivdarr rdngsyffrm |
| 121 | ergstkysyk spqlsvhvtd lthrpkilip gtlepghskn ltcsvswace qgtppifswl |
| 181 | saaptslgpr tthssvliit prpqdhgtnl tcqvkfagag vttertiqln vtyvpqnptt |
| 241 | gifpgdgsgk qetragvvhg aiggagvtal lalclcliff ivkthrrkaa rtavgrndth |
| 301 | pttgsaspvr |

SEQ ID NO: 87 Human LST1 Transcript Variant 1 cDNA Sequence (NM_007161.3;
CDS: 101-415)

| | |
|---|---|
| 1 | atgaggaact tgaggcaagt caccagcccc tgatcatttc gcctaaaaga gcaaggacta |
| 61 | gagttcctga cctccaggcc agtccctgat ccctgaccta atgttatcgc ggaatgatga |
| 121 | tatatgtatc tacgggggcc tggggctggg cgggctcctg cttctggcag tggtccttct |
| 181 | gtccgcctgc ctgtgttggc tgcatcgaag agtaaagagg ctggagagga gctggcacct |
| 241 | tctgtcgtgg tccgaggccc agggctcctc agagcaggaa ctccactatg catctctgca |
| 301 | gaggctgcca gtgcccagca gtgagggacc tgacctcagg ggcagagaca agagaggcac |
| 361 | caaggaggat ccaagagctg actatgcctg cattgctgag aacaaaccca cctgagcacc |
| 421 | ccagacacct tcctcaaccc aggcgggtgg acagggtccc cctgtggtcc agccagtaaa |
| 481 | aaccatggtc cccccacttc tgtgtctcag tcctctcagt ccatctcgag cctccgttca |
| 541 | aattgatcat catcaaaact tatgtggctt tttgaccttt gaatagggaa ttttttaaat |
| 601 | tttttaaaaa ttaaaataaa aaaaacacat ggctcaccct tccacccaaa aaa |

SEQ ID NO: 88 Human LST1 Isoform 1 Amino Acid Sequence (NP_009092.3)

| | |
|---|---|
| 1 | mlsrnddici ygglglggll llavvllsac lcwlhrrvkr lerswhllsw sqaqgsseqe |
| 61 | lhyaslqrlp vpssegpdlr grdkrgtked pradyaciae nkpt |

SEQ ID NO: 89 Human LST1 Transcript Variant 2 cDNA Sequence (NM_205837.2;
CDS: 210-410)

| | |
|---|---|
| 1 | acttcagccc tagcagcatc tgcctgtggg aagcagctct ccacaccagc caagggggcc |
| 61 | cccacactcc cgcgctgctc tgcggctcag ggagcagccc acctgctgga tgaggaactt |
| 121 | gaggcaagtc accagcccct gatcatttcg cctaaaagag caaggactag agttcctgac |
| 181 | ctccaggcca gtccctgatc cctgacctaa tgttatcgcg gaatgatgat atatgtatct |
| 241 | acgggggcct ggggctgggc gggctcctgc ttctggcagt ggtccttctg tccgcctgcc |
| 301 | tgtgttggct gcatcgaaga gcaccttctg tcctggtccc aggcccaggg ctcgtcagag |
| 361 | caggaactcc actatgcatc tctgcagagg ctgccagtgc ccagcagtga gggacctgac |
| 421 | ctcaggggca gagacaagag aggcaccaag gaggatccaa gagctgacta tgcctgcatt |
| 481 | gctgagaaca aacccacctg agcaccccag acaccttcct caacccaggc gggtggacag |
| 541 | ggtccccctg tggtccagcc agtaaaaacc atggtccccc cacttctgtg tctcagtcct |
| 601 | ctcagtccat ctcgagcctc cgttcaaatt gatcatcatc aaaacttatg tggctttttg |
| 661 | acctttgaat agggaatttt ttaaattttt taaaaattaa aataaaaaaa acacatggct |
| 721 | cacccttcca cccaaaaaa |

SEQ ID NO: 90 Human LST1 Isoform 2 Amino Acid Sequence (NP_995309.2)

| | |
|---|---|
| 1 | mlsrnddici ygglglggll llavvllsac lcwlhrraps vlvpgpgllr agtplcisae |
| 61 | aasaqq |

SEQ ID NO: 91 Human LST1 Transcript Variant 3 cDNA Sequence (NM_205838.2;
CDS: 238-438)

| | |
|---|---|
| 1 | gctggggagg aacccaggct gggggagaag ttaaagccag aggaggggca ggaatgtctg |
| 61 | aggtggcaac acttctcttc agccagacag cactggccag tttggagtct gtccatcctg |
| 121 | caggccacaa gctctggatg aggaacttga ggcaagtcac cagcccctga tcatttcgcc |
| 181 | taaaagagca aggactagag ttcctgacct ccaggccagt ccctgatccc tgacctaatg |
| 241 | ttatcgcgga atgatgtaaa gaggctggag aggagctggg cccagggctc ctcagagcag |
| 301 | gaactccact atgcatctct gcagaggctg ccagtgccca gcagtgaggg acctgacctc |
| 361 | aggggcagag acaagagagg caccaaggag gatccaagag ctgactatgc ctgcattgct |
| 421 | gagaacaaac ccacctgagc accccagaca ccttcctcaa cccaggcggg tggacagggt |
| 481 | ccccctgtgg tccagccagt aaaaaccatg gtccccccac ttctgtgtct cagtcctctc |
| 541 | agtccatctc gagcctccgt tcaaattgat catcatcaaa acttatgtgg ctttttgacc |
| 601 | tttgaatagg gaatttttta aattttttaa aaattaaaat aaaaaaaaca catggctcac |
| 661 | ccttccaccc aaaaaa |

SEQ ID NO: 92 Human LST1 Isoform 3 Amino Acid Sequence (NP_995310.2)

| | |
|---|---|
| 1 | mlsrndvkrl erswaqgsse qelhyaslqr lpvpssegpd lrgrdkrgtk edpradyaci |
| 61 | aenkpt |

SEQ ID NO: 93 Human LST1 Transcript Variant 4 cDNA Sequence (NM_205839.2;
CDS: 238-531)

| | |
|---|---|
| 1 | gctggggagg aacccaggct gggggagaag ttaaagccag aggaggggca ggaatgtctg |
| 61 | aggtggcaac acttctcttc agccagacag cactggccag tttggagtct gtccatcctg |
| 121 | caggccacaa gctctggatg aggaacttga ggcaagtcac cagcccctga tcatttcgcc |
| 181 | taaaagagca aggactagag ttcctgacct ccaggccagt ccctgatccc tgacctaatg |
| 241 | ttatcgcgga atgatgatat atgtatctac gggggcctgg ggctgggcgg gctcctgctt |
| 301 | ctggcagtgg tccttctgtc cgcctgcctg tgttggctgc atcgaagagt aaagaggctg |
| 361 | gagaggagct gggcccaggg ctcctcagag caggaactcc actatgcatc tctgcagagg |
| 421 | ctgccagtgc ccagcagtga gggacctgac ctcaggggca gagacaagag aggcaccaag |
| 481 | gaggatccaa gagctgacta tgcctgcatt gctgagaaca aacccacctg agcaccccag |
| 541 | acaccttcct caacccaggc gggtggacag ggtccccctg tggtccagcc agtaaaaacc |
| 601 | atggtccccc cacttctgtg tctcagtcct ctcagtccat ctcgagcctc cgttcaaatt |
| 661 | gatcatcatc aaaacttatg tggctttttg acctttgaat agggaatttt ttaaattttt |
| 721 | taaaaattaa aataaaaaaa acacatggct cacccttcca cccaaaaaa |

SEQ ID NO: 94 Human LST1 Isoform 4 Amino Acid Sequence (NP_995311.2)

| | |
|---|---|
| 1 | mlsrnddici ygglglggal llavvllsac lcwlhrrvkr lerswaqqss eqelhyaslq |
| 61 | rlpvpssegp dlrgrdkrgt kedpradyac iaenkpt |

SEQ ID NO: 95 Human LST1 Transcript Variant 5 cDNA Sequence (NM_205840.2;
CDS: 101-280)

| | |
|---|---|
| 1 | atgaggaact tgaggcaagt caccagcccc tgatcatttc gcctaaaaga gcaaggacta |
| 61 | gagttcctga cctccaggcc agtccctgat ccctgaccta atgttatcgc ggaatgatga |
| 121 | tatatgtatc tacgggggcc tggggctggg cgggctcctg cttctggcag tggtccttct |
| 181 | gtccgcctgc ctgtgttggc tgcatcgaag aggcccaggg ctcctcagag caggaactcc |
| 241 | actatgcatc tctgcagagg ctgccagtgc ccagcagtga gggacctgac ctcaggggca |
| 301 | gagacaagag aggcaccaag gaggatccaa gagctgacta tgcctgcatt gctgagaaca |
| 361 | aacccacctg agcaccccag acaccttcct caacccaggc gggtggacag ggtccccctg |
| 421 | tggtccagcc agtaaaaacc atggtccccc cacttctgtg tctcagtcct ctcagtccat |
| 481 | ctcgagcctc cgttcaaatt gatcatcatc aaaacttatg tggctttttg acctttgaat |
| 541 | agggaatttt ttaaattttt taaaaattaa aataaaaaaa acacatggct cacccttcca |
| 601 | cccaaaaaa |

SEQ ID NO: 96 Human LST1 Isoform 5 Amino Acid Sequence (NP_995312.2)

| | |
|---|---|
| 1 | mlsrnddici ygglglggll llavvllsac lcwlhrrgpg llragtplci saeaasaqq |

SEQ ID NO: 97 Human LST1 Transcript Variant 6 cDNA Sequence
(NM_001166538.1; CDS: 101-322)

| | |
|---|---|
| 1 | atgaggaact tgaggcaagt caccagcccc tgatcatttg gcctaaaaga gcaaggacta |
| 61 | gagttcctga cctccaggcc agtcgctgat cgctgaccta atgttatcgc ggaatgatgt |
| 121 | aaagaggctg gagaggagct ggcaccttct gtcctggtcc caggcccagg gctcctcaga |
| 181 | gcaggaactc cactatgcat ctctgcagag gctgccagtg cccagcagtg agggacctga |
| 241 | cctcaggggc agagacaaga gaggcaccaa ggaggatcca agagctgact atgcctgcat |
| 301 | tgctgagaac aaacccacct gagcacccca gacaccttcc tcaacccagg cgggtggaca |
| 361 | gggtccccct gtggtccagc cagtaaaaac catggtcccc ccacttctgt gtctcagtgc |
| 421 | tctcagtcca tctcgagcct ccgttcaaat tgatcatcat caaaacttat gtggcttttt |
| 481 | gacctttgaa tagggaattt tttaaatttt ttaaaaatta aaataaaaaa aacacatggc |
| 541 | tcacccttcc acccaaaaaa |

SEQ ID NO: 98 Human LST1 Isoform 6 Amino Acid Sequence (NP_001160010.1)

| | |
|---|---|
| 1 | mlsrndvkrl erswhllsws qaqgsseqel hyaslqrlpv pssegpdlrg rdkrgtkedp |
| 61 | radyaciaen kpt |

SEQ ID NO: 99 Human TNFAIP8L2 cDNA Sequence (NM_024575.4; CDS: 127-681)

| | |
|---|---|
| 1 | ggccaagcca aagggctctc acactaagtg aagcttctcc attctgtaag ctttccggga |
| 61 | acatccaagg caagactggc acccagcaca gcagtgactg accacatacc ccactctcca |
| 121 | ggacccatgg agtccttcag ctcaaagagc ctggcactgc aagcagagaa gaagctactg |
| 181 | agtaagatgg cgggtcgctc tgtggctcat ctcttcatag atgagacaag cagtgaggtg |
| 241 | ctagatgagc tctaccgtgt gtccaaggag tacacgcaca gccggcccca ggcccagcgc |
| 301 | gtgatcaagg acctgatcaa agtggccatc aaggtggctg tgctgcaccg caatggctcc |
| 361 | tttggcccca gtgagctggc cctggctacc cgctttcgcc agaagctgcg gcagggtgcc |
| 421 | atgacggcac ttagctttgg tgaggtagac ttcaccttcg aggctgctgt tctggctggc |
| 481 | ctgctgaccg agtgccggga tgtgctgcta gagttggtgg aacaccacct cacgcccaag |
| 541 | tcacatggcc gcatccgcca cgtgtttgat cacttctctg acccaggtct gctcacggcc |
| 601 | ctctatgggc ctgacttcac tcagcacctt ggcaagatct gtgacggact caggaagctg |
| 661 | ctagacgaag ggaagctctg agagccctga gcctagcaca ttccaccttg acaaaatggt |
| 721 | tgactgagaa aacacagata atgggcttcc taaccctgct cacctggcac taacactttt |
| 781 | caatcttcag gcttcattcc ttcccaagag tgcttttgac tctgagacca gcccaccccc |
| 841 | aaacagctag tggagaagga gcaatgctga ggggtgaggc ctctctccca ctccagcccc |
| 901 | aggacaggaa acagaactgc ctgaaaaagg tgaagtgaaa cttggatctc tatttctccc |
| 961 | ataagggact tctgaaacag ggaagccccc tcccatgtga accaaggaaa ggaggcacag |
| 1021 | cccagagaac ccctttgggg atactaaaga cagaagaggg gaaggtggcc cttagagaca |
| 1081 | gagcttggac agatgccaga ggctctgttc cagagtgcag gaagaagggg ctagggcagg |
| 1141 | ggagattctc ataggggaaa taaaactact aaaatatgaa aaaaaaaaaa aaaaaaaaa |

SEQ ID NO: 100 Human TNFAIP8L2 Amino Acid Sequence (NP_078851.2)

| | |
|---|---|
| 1 | mesfssksla lqaekkllsk magrsvahlf idetssevld elyrvskeyt hsrpqaqrvi |
| 61 | kdlikvaikv avlhrngsfg pselalatrf rqklrqgamt alsfgevdft feaavlagll |
| 121 | tecrdvllel vehhltpksh grirhvfdhf sdpglltaly gpdftqhlgk icdglrklld |
| 181 | egkl |

SEQ ID NO: 101 Mouse TNFAIP8L2 cDNA Sequence (NM_027206.2; CDS: 93-647)

| | |
|---|---|
| 1 | gctctcagaa acatccaagg ccagactggc acccagcaca cggcagccgg ttgcctcttc |
| 61 | cagtgactga tcacacaccg accgcaagca ccatggagtc cttcagctca aagagtctgg |
| 121 | cactacaagc ggagaagaag ctgctgagta aaatggctgg tcggtccgtg gcgcatctct |
| 181 | ttatcgacga gaccagcagc gaggtgctag acgagcttta ccgcgtgtcc aaagaataca |
| 241 | cgcacagccg gcccaaggca cagcgggtga tcaaagacct catcaaggta gcggttaaag |
| 301 | tggctgtgct gcaccgcagt ggctgctttg gccctgggga gctggctctg gctacacgat |
| 361 | ttcgtcagaa gctacggcag ggcgccatga ccgcacttag cttcggtgag gtggacttca |
| 421 | cctttgaggc tgccgtgcta gcaggtctgc tcgtcgagtg ccgggacatt ctgctggagc |
| 481 | tggtggagca ccacctcaca cccaagtcac atgaccgcat caggcacgtg tttgatcact |
| 541 | actctgaccc cgacdtgctg gctgccctct atgggcctga cttcactcag caccttggca |
| 601 | agatctgtga tgggctccgg aagctgctgg acgagggcaa gctctgaagc tccggagctc |
| 661 | agcacactgg actttggcaa aatgactgac tgggaaatga cacatcgggc tctctaaccc |
| 721 | tgcaccaatg cttctcgatc ccctggcttc actctctccc aagcgtgcta tagacactga |
| 781 | ggccaccccc acccccaaac tactgctggg gcaaagcaag gctgaactga gtgaggcggc |
| 841 | tctcgctccc atccagttcc agtaaaggaa acagctgact gaagaagaag tgaaactcag |
| 901 | gtccgcttct cccgggaggg ctgcccagaa ccgggggatc ctcctcctcc acaaacccga |
| 961 | ggaaggggac actaaaggcc acacgcagga aggtggtcat tagagacctc ctggatctag |
| 1021 | agttggaggc agcactagga cacatacaga cgctgtgttc cagagcttct agaaggagct |
| 1081 | gggggaaggg aggcacaggg gaaataaaac cactaaagca tg |

SEQ ID NO: 102 Mouse TNFAIP8L2 Amino Acid Sequence (NP_081482.1)

| | |
|---|---|
| 1 | mesfssksla lqaekkllsk magrsvahlf idetssevld elyrvskeyt hsrpkaqrvi |
| 61 | kdlikvavkv avlhrsgcfg pgelalatrf rqklrqgamt alsfgevdft feaavlagll |
| 121 | vecrdillel vehhltpksh drirhvfdhy sdpdllaaly gpdftghlgk icdglrklld |
| 181 | egkl |

SEQ ID NO: 103 Human SPI1 Transcript Variant 1 cDNA Sequence
(NM_001080547.1; CDS: 224-1039)

| | |
|---|---|
| 1 | gactatctcc cagcggcagg cccttcgata aaatcaggaa cttgtgctgg ccctgcaatg |
| 61 | tcaagggagg gggctcaccc agggctcctg tagctcaggg ggcaggcctg agccctgcac |
| 121 | ccgccccacg accgtccagc ccctgacggg gcaccccatc ctgaggggct ctgcattggc |
| 181 | ccccaccgag gcaggggatc tgaccgactc ggagcccggc tggatgttac aggcgtgcaa |
| 241 | aatggaaggg tttcccctcg tcccccctca gccatcagaa gacctggtgc cctatgacac |
| 301 | ggatctatac caacgccaaa cgcacgagta ttacccctat ctcagcagtg atggggagag |
| 361 | ccatagcgac cattactggg acttccaccc ccaccacgtg cacagcgagt tcgagagctt |
| 421 | cgccgagaac aacttcacgg agctccagag cgtgcagccc ccgcagctgc agcagctcta |
| 481 | ccgccacatg gagctggagc agatgcacgt cctcgatacc cccatggtgc caccccatcc |
| 541 | cagtcttggc caccaggtct cctacctgcc ccggatgtgc ctccagtacc catccctgtc |
| 601 | cccagcccag cccagctcag atgaggagga gggcgagcgg cagagccccc cactggaggt |
| 661 | gtctgacggc gaggcggatg gcctggagcc cgggcctggg ctcctgcctg gggagacagg |
| 721 | cagcaagaag aagatccgcc tgtaccagtt cctgttggac ctgctccgca gcggcgacat |
| 781 | gaaggacagc atctggtggg tggacaagga caagggcacc ttccagttct cgtccaagca |
| 841 | caaggaggcg ctggcgcacc gctggggcat ccagaagggc aaccgcaaga agatgaccta |
| 901 | ccagaagatg gcgcgcgcgc tgcgcaacta cggcaagacg ggcgaggtca agaaggtgaa |
| 961 | gaagaagctc acctaccagt tcagcggcga agtgctgggc cgcgggggcc tggccgagcg |
| 1021 | gcgccacccg ccccactgag cccgcagccc ccgccgggcc ccgccaggcc tccccgctgg |
| 1081 | ccatagcatt aagccctcgc ccggcccgga cacagggagg acgctcccgg ggcccagagg |
| 1141 | caggactgtg gcgggccggg cctcgcctca cccgccccct ccccccactc caggccccct |
| 1201 | ccacatcccg cttcgcctcc ctccaggact ccacccrggc tccrggacgc cagctgggcg |
| 1261 | tcagacccca ccggggcaac cttgcagagg acgacccggg gtactgcctt gggagtctca |
| 1321 | agtccgtatg taaatcagat ctcccctctc acccctccca cccattaacc tcctcccaaa |
| 1381 | aaacaagtaa agttattctc aatccatcaa aaaaaaaaaa aaaaaa |

SEQ ID NO: 104 Human SPI1 Isoform 1 Amino Acid Sequence (NP_001074016.1)

| | |
|---|---|
| 1 | mlqackmegf plvppqpsed lvpydtdlyq rqtheyypyl ssdgeshsdh ywdfhphhvh |
| 61 | sefesfaenn ftelqsvgpp qlqqlyrhme leqmhvldtp mvpphpslgh qvsylprmcl |
| 121 | qypslspaqp ssdeeegerq spplevsdge adglepgpgl lpgetgskkk irlyqflldl |
| 181 | lrsgdmkdsi wwvdkdkgtf qfsskhkeal ahrwgiqkgn rkkmtyqkma ralrnygktg |
| 241 | evkkvkkklt yqfsgevlgr gglaerrhpp h |

SEQ ID NO: 105 Human SPI1 Transcript Variant 2 cDNA Sequence (NM_003120.2;
CDS: 224-1036)

| | |
|---|---|
| 1 | gactatctcc cagcggcagg cccttcgata aaatcaggaa cttgtgctgg ccctgcaatg |
| 61 | gggctcaccc agggctcctg tagctcaggg ggcaggcctg agccctgcac agccctgcac |
| 121 | ccgccccacg accgtccagc ccctgacggg gcaccccatc ctgaggggct ctgcattggc |
| 181 | ccccaccgag gcaggggatc tgaccgactc ggagcccggc tggatgttac aggcgtgcaa |
| 241 | aatggaaggg tttcccctcg tcccccctcc atcagaagac ctggtgccct atgacacgga |
| 301 | tctataccaa cgccaaacgd acgagtatta cccctatctc agcagtgatg gggagagcca |
| 361 | tagcgaccat tactgggact tccaccccca ccacgtgcac agcgagttcg agagcttcgc |
| 421 | cgagaacaac ttcacggagc tccagagcgt gcagcccccg cagctgcagc agctctaccg |
| 481 | ccacatggag ctggagcaga tgcacgtcct cgataccccc atggtgccac cccatcccag |
| 541 | tcttggccac caggtctcct acctgccccg gatgtgcctc cagtacccat ccctgtcccc |
| 601 | agcccagccc agctcagatg aggaggaggg cgagcggcag agccccccac tggaggtgtc |
| 661 | tgacggcgag gcggatggcc tggagcccgg gcctgggctc ctgcctgggg agacaggcag |
| 721 | caagaagaag atccgcctgt accagttcct gttggacctg ctccgcagcg gcgacatgaa |
| 781 | ggacagcatc tggtgggtgg acaaggacaa gggcaccttc cagttctcgt ccaagcacaa |
| 841 | ggcggcgctg gcgcaccgct ggggcatcca gaagggcaac cgcaagaaga tgacctacca |
| 901 | gaagatggcg cgcgcgctgc gcaactacgg caagacgggc gaggtcaaga aggtgaagaa |
| 961 | gaagctcacc taccagttca gcggcgaagt gctgggccgc gggggcctgg ccgagcggcg |
| 1021 | ccacccgccc cactgagccc gcagcccccg ccgggccccg ccaggcctcc ccgctggcca |
| 1081 | tagcattaag ccctcgcccg gcccggacac agggaggacg ctcccggggc ccagaggcag |
| 1141 | gactgtggcg ggccgggcct cgcctcaccc gccccctccc cccactccag gccccctcca |
| 1201 | catcccgctt cgcctccctc caggactcca ccccggctcc cggacgccag ctgggcgtca |
| 1261 | gaccccaccg gggcaacctt gcagaggacg acccggggta ctgccttggg agtctcaagt |
| 1321 | ccgtatgtaa atcagatctc ccctctcacc cctcccaccc attaacctcc tcccaaaaaa |
| 1381 | caagtaaagt tattctcaat ccatcaaaaa aaaaaaaaaa aaa |

SEQ ID NO: 106 Human SPI1 Isoform 2 Amino Acid Sequence (NP_003111.2)

| | |
|---|---|
| 1 | mlqackmegf plvpppsedl vpydtdlyqr qtheyypyls sdgeshsdhy wdfhphhvhs |
| 61 | efeefaennf telqsvqppq lqqlyrhmel eqmhvldtpm vpphpslghq vsylprmclq |
| 121 | ypslspaqps sdeeegerqs pplevsdgea dglepgpgll pgetgskkki rlyqflldll |
| 181 | rsgdmkdsiw wvdkdkgtfq fsskhkeala hrwgiqkgnr kkmtyqkmar alrnygktge |
| 241 | vkkvkkklty qfsgevlgrg glaerrhpph |

SEQ ID NO: 107 Mouse SPI1 cDNA Sequence (NM_011355.2; CDS: 272-1090)

| | |
|---|---|
| 1 | aagagattta tgcaaacggg ctggggcggt gatgtcaccc caaggggact atctcccagt |
| 61 | ggcaggccct tcgataaaat caggaacttg tgctggccct gcaatgtcaa gggagggggc |
| 121 | tcacccaggg ctcctgtagc tcagggggca ggcctgagcc ctgcgtctga cccacgaccg |
| 181 | tccagtcccc cgacggggca cctggtcctg agggggatcc gccttgatcc ccaccgaagc |
| 241 | aggggatctg accaacctgg agctcagctg gatgttacag gcgtgcaaaa tggaagggtt |
| 301 | ttccctcacc gcccctccat cggatgactt ggttacttac gattcagagc tataccaacg |
| 361 | tccaatgcat gactactact ccttcgtggg cagcgatgga gaaagccata gcgatcacta |
| 421 | ctgggatttc tccgcacacc atgtccacaa caacgagttt gagcccttcc ctgagaacca |
| 481 | cttcacagag ctgcagagtg tgcagccccc gcagctacag cagctctatc gccacatgga |
| 541 | gctggaacag atgcacgtcc tcgatactcc catggtgcca ccccacaccg gcctcagtca |
| 601 | ccaggtttcc tacatgcccc ggatgtgctt cccttatcaa accttgtccc cagcccacca |
| 661 | gcagagctca gatgaggagg agggtgagag gcagagccct cccctggagg tgtatgatgg |
| 721 | agaagctgat ggcttggagc ctgggccagg tcttctgcac ggggagacag gcagcaagaa |
| 781 | aaagattcgc ctgtaccagt tcctgctgga cctgctgcgc agcggcgaca tgaaggacag |
| 841 | catctggtgg gtggacaagg acaaaggtac cttccagttc tcgtccaagc acaaggaggc |
| 901 | gctggcgcac cgctggggca tccagaaggg caaccgcaag aagctgacct accagaagat |
| 961 | ggcgcgcgcg ctgcgcaact acggcaagac aggcgaggtg aagcaagtca agaagaagct |
| 1021 | cacctaccag ttcagcggcg aggtgctggg ccgtgggggc ctggccgagc ggcgcctccc |
| 1081 | gccccactga tcgcccgcag agaccgccag gctcctggac cccgccggcc atagcattaa |
| 1141 | cccgtcgccc ggcccggaca cagggaggac attcccaggg ccgaggcagg actgggggcc |
| 1201 | cggcctcgcc ctcccatgcc cggcctggcc cgccccaccc gctttgcctc ccaccaggac |
| 1261 | tctagcccgc tccaagggcc gcctgggcct cggacctcaa ccgagggtca gcctggctta |
| 1321 | gtggccacgg tgcttccttg ggagtctggc gctggcacct ttttgtatat tgaatgcttt |
| 1381 | ttaaaaagct cttcctcccc accccctcat tagtcactaa agacaagtaa aattattgac |
| 1441 | agctattctc ccagaaaaaa aaaaaaaaaa aaa |

SEQ ID NO: 108 Mouse SPI1 Amino Acid Sequence (NP_035485.1)

| | |
|---|---|
| 1 | mlqackmegf sltappsddl vtydselyqr pmhdyysfvg sdgeshsdhy wdfsahhvhn |
| 61 | nefenfpenh ftelqsvqpp qlqqlyrhme leqmhviltp mvpphtglsh qvsvmprmcf |
| 121 | pyqtlspahq qssdeeeger qspplevsdg eadglepgpg llhgetgskk kirlyqflld |
| 781 | llrsgdmkds iwwvdkdkgt fqfsskhkea lahrwgiqkg nrkkmtyqkm aralrnygkt |
| 241 | gevkkvkkkl tyqfsgevlg rgglaerrlp ph |

SEQ ID NO: 109 Human LILRB2 Transcript Variant 1 cDNA Sequence
(NM_005874.4; CDS: 267-2063)

| | |
|---|---|
| 1 | atttggttga aagaaaaccc acaatccagt gtcaagaaag aagtcaactt ttcttcccct |

w

| | |
|---|---|
| 61 | acttccctgc atttctcctc tgtgctcact gccacacaca gctcaacctg gacagcacag |
| 121 | ccagaggcga gatgcttctc tgctgatctg agtctgcctg cagcatggac ctgggtcttc |
| 181 | cctgaagcat ctccagggct ggagggacga ctgccatgca ccgagggctc atccatccgc |
| 241 | agagcagggc agtgggaggg gacgccatga cccccatcgt cacagtcctg atctgtctcg |
| 301 | ggctgagtct gggccccagg acccgcgtgc agacagggac catccccaag cccaccctgt |
| 361 | gggctgagcc agactctgtg atcacccagg ggagtcccgt caccctcagt tgtcagggga |
| 421 | gccttgaagc ccaggagtac cgtctatata gggagaaaaa atcagcatct tggattacac |
| 481 | ggatacgacc agagcttgtg aagaacggcc agttccacat cccatccatc acctgggaac |
| 541 | acacagggcg atatggctgt cagtattaca gccgcgctcg gtggtctgag ctcagtgacc |
| 601 | ccctggtgct ggtgatgaca ggagcctacc caaaacccac cctctcagcc cagcccagcc |
| 661 | ctgtggtgac ctcaggagga agggtgaccc tccagtgtga gtcacaggtg gcatttggcg |
| 721 | gcttcattct gtgtaaggaa ggagaagatg aacacccaca atgcctgaac tcccagcccc |
| 781 | atgcccgtgg gtcgtcccgc gccatcttct ccgtgggccc cgtgagcccg aatcgcaggt |
| 841 | ggtcgcacag gtgctatggt tatgacttga actctcccta tgtgtggtct tcacccagtg |
| 901 | atctcctgga gctcctggtc ccaggtgttt ctaagaagcc atcactctca gtgcagccgg |
| 961 | gtcctgtcat ggcccctggg gaaagcctga ccctccagtg tgtctctgat gtcggctatg |
| 1021 | acagatttgt tctgtacaag gagggggaac gtgaccttcg ccagctccct ggccggcagc |
| 1081 | cccaggctgg gctctcccag gccaacttca ccctgggccc tgtgagccgc tcctacgggg |
| 1141 | gccagtacag atgctacggt gcacacaacc tctcctctga gtgctcggcc cccagcgacc |
| 1201 | ccctggacat cctgatcaca ggacagatcc gtggcacacc cttcatctca gtgcagccag |
| 1261 | gccccacagt ggcctcagga gagaacgtga ccctgctgtg tcagtcatgg cggcagttcc |
| 1321 | acactttcct tctgaccaag gcgggagcag ctgatgcccc actccgtcta agatcaatac |
| 1381 | acgaatatcc taagtaccag gctgaattcc ccatgagtcc tgtgacctca gcccacgcgg |
| 1441 | ggacctacag gtgctacggc tcactcaact ccgaccccta cctgctgtct caccccagtg |
| 1501 | agcccctgga gctcgtggtc tcaggaccct ccatgggttc cagcccccca cccaccggtc |
| 1561 | ccatctccac acctgcaggc cctgaggacc agcccctcac ccccactggg tcggatcccc |
| 1621 | aaagtggtct gggaaggcac ctgggggttg tgatcggcat cttggtggcc gtcgtcctac |
| 1681 | tgctcctcct cctcctcctc ctcttcctca tcctccgaca tcgacgtcag ggcaaacact |
| 1741 | ggacatcgac ccagagaaag gctgatttcc aacatcctgc aggggctgtg gggccagagc |
| 1801 | ccacagacag aggcctgcag tggaggtcca gcccagctgc cgacgcccag gaagaaaacc |
| 1861 | tctatgctgc cgtgaaggac acacagcctg aagatggggt ggagatggac actcgggctg |
| 1921 | ctgcatctga agccccccag gatgtgacct acgcccagct gcacagcttg accctcagac |
| 1981 | ggaaggcaac tgagcctcct ccatcccagg aaagggaacc tccagctgag cccagcatct |
| 2041 | acgccaccct ggccatccac tagcccggag ggtacgcaga ctccacactc agtagaagga |
| 2101 | gactcaggac tgctgaaggc acgggagctg cccccagtgg acaccaatga accccagtca |
| 2161 | gcctggaccc ctaacaaaga ccatgaggag atgctgggaa ctttgggact cacttgattc |
| 2221 | tgcagtcgaa ataactaata tccctacatt ttttaattaa agcaacagac ttctcaataa |
| 2281 | tcaatgagtt aaccgagaaa actaaaatca gaagtaagaa tgtgctttaa actgaatcac |
| 2341 | aatataaata ttacacatca cacaatgaaa ttgaaaaagt acaaaccaca aatgaaaaaa |
| 2401 | gtagaaacga aaaaaaaaaa ctaggaaatg aatgacgttg gctttcgtat aaggaattta |
| 2461 | gaaaaagaat aaccaattat tccaaatgaa ggtgtaagaa agggaataag aagaagaaga |
| 2521 | gttgctcatg aggaaaaacc aaaacttgaa aattcaacaa agccaatgaa gctcattctt |
| 2581 | gaaaatatta attacagtca taaatcctaa ctacattgag caagagaaag aaagagcagg |
| 2641 | cacgcatttc catatgggag tgagccagca gacagcccag cagatcctac acacattttc |
| 2701 | acaaactaac cccagaacag gctgcaaacc tataccaata tactagaaaa tgcagattaa |
| 2761 | atggatgaaa tattcaaaac tggagtttac ataatgaacg taagagtaat cagagaatct |
| 2821 | gactcatttt aaatgtgtgt gtatgtgtgt gtatatatat gtgtgtgtgt gtgtgtgtgt |
| 2881 | gtgtgtgtga aaaacattga ctgtaataaa aatgttccca tcgtaaaaaa aaaaaaaaa |

SEQ ID NO: 110 Human LILRB2 Isoform 1 Amino Acid Sequence (NP_005865.3)

| | |
|---|---|
| 1 | mtpivtvlic lglslgprtr vqtgtipkpt lwaepdsvit qgspvtlscq gsleaqeyrl |
| 61 | yrekksaswi trirpelvkn gqfhipsitw ehtgrygcqy ysrarwsels dplvlvmtga |
| 121 | ypkptlsaqp spvvtsggrv tlqcesqvaf ggfilckege dehpqclnsq phargssrai |
| 181 | fsvgpvspnr rwshrcygyd lnspyvwssp sdllellvpg vskkpslsyq pgpvmapges |
| 241 | ltlqcvsdvg ydrfvlykeg erdlrqlpqr qpqaglsqan ftlgpvsrsy qgqyrcygah |
| 301 | nlssecsaps dpldilitgq irgtpfisvq pgptvasgen vtllcqswrq fhtflltkag |
| 361 | aadaplrlrs iheypkyqae fpmspvtsah agtyrcygsl nsdpyllshp seplelvvsg |
| 421 | psmgsspppt gpistpagpe dqpltptgsd pqsglgrhlg vvigilvavv lllllllllf |
| 481 | lilrhrrqgk hwtstqrkad fqhpagaygp eptdrglqwr sspaadaqee nlyaavkdtq |
| 541 | pedgvemdtr aaaseapqdv tyaqlhsltl rrkateppps qereppaeps iyatlaih |

SEQ ID NO: 111 Human LILRB2 Transcript Variant 2 cDNA Sequence
(NM_001080978.3; CDS: 267-2060)

| | |
|---|---|
| 1 | atttggttga aagaaaaccc acaatccagt gtcaagaaag aagtcaactt ttcttcccct |
| 61 | acttccctgc atttctcctc tgtgctcact gccacacaca gctcaacctg gacagcacag |
| 121 | ccagaggcga gatgcttctc tgctgatctg agtctgcctg cagcatggac ctgggtcttc |
| 181 | cctgaagcat ctccagggct ggagggacga ctgccatgca ccgagggctc atccatccgc |
| 241 | agagcagggc agtgggagga gacgccatga cccccatcgt cacagtcctg atctgtctcg |
| 301 | ggctgagtct gggccccagg acccgcgtgc agacagggac catccccaag cccaccctgt |
| 361 | gggctgagcc agactctgtg atcacccagg ggagtcccgt caccctcagt tgtcagggga |
| 421 | gccttgaagc ccaggagtac cgtctatata gggagaaaaa atcagcatct tggattacac |
| 481 | ggatacgacc agagcttgtg aagaacggcc agttccacat cccatccatc acctgggaac |
| 541 | acacagggcg atatggctgt cagtattaca gccgcgctcg gtggtctgag ctcagtgacc |
| 601 | ccctggtgct ggtgatgaca ggagcctacc caaaacccac cctctcagcc cagcccagcc |
| 661 | ctgtggtgac ctcaggagga agggtgaccc tccagtgtga gtcacaggtg gcatttggcg |
| 721 | gcttcattct gtgtaaggaa ggagaagatg aacacccaca atgcctgaac tcccagcccc |
| 781 | atgcccgtgg gtcgtcccgc gccatcttct ccgtgggccc cgtgagcccg aatcgcaggt |
| 841 | ggtcgcacag gtgctatggt tatgacttga actctcccta tgtgtggtct tcacccagtg |
| 901 | atctcctgga gctcctggtc ccaggtgttt ctaagaagcc atcactctca gtgcagccgg |
| 961 | gtcctgtcat ggcccctggg gaaagcctga ccctccagtg tgtctctgat gtcggctatg |
| 1021 | acagatttgt tctgtacaag gagggggaac gtgaccttcg ccagctccct ggccggcagc |
| 1081 | cccaggctgg gctctcccag gccaacttca ccctgggccc tgtgagccgc tcctacgggg |
| 1141 | gccagtacag atgctacggt gcacacaacc tctcctctga gtgctcggcc cccagcgacc |
| 1201 | ccctggacat cctgatcaca ggacagatcc gtggcacacc cttcatctca gtgcagccag |
| 1261 | gccccacagt ggcctcagga gagaacgtga ccctgctgtg tcagtcatgg cggcagttcc |
| 1321 | acactttcct tctgaccaag gcgggagcag ctgatgcccc actccgtcta agatcaatac |
| 1381 | acgaatatcc taagtaccag gctgaattcc ccatgagtcc tgtgacctca gcccacgcgg |
| 1441 | ggacctacag gtgctacggc tcactcaact ccgaccccta cctgctgtct caccccagtg |
| 1501 | agcccctgga gctcgtggtc tcaggaccct ccatgggttc cagcccccca cccaccggtc |
| 1561 | ccatctccac acctggccct gaggaccagc ccctcacccc cactgggtcg gatccccaaa |
| 1621 | gtggtctggg aaggcacctg ggggttgtga tcggcatctt ggtggccgtc gtcctactgc |
| 1681 | tcctcctcct cctcctcctc ttcctcatcc tccgacatcg acgtcagggc aaacactgga |
| 1741 | catcgaccca gagaaaggct gatttccaac atcctgcagg ggctgtgggg ccagagccca |
| 1801 | cagacagagg cctgcagtgg aggtccagcc cagctgccga cgcccaggaa gaaaacctct |
| 1861 | atgctgccgt gaaggacaca cagcctgaag atggggtgga gatggacact cgggctgctg |
| 1921 | catctgaagc cccccaggat gtgacctacg cccagctgca cagcttgacc ctcagacgga |
| 1981 | aggcaactga gcctcctcca tcccaggaaa gggaacctcc agctgagccc agcatctacg |
| 2041 | ccaccctggc catccactag cccggagggt acgcagactc cacactcagt agaaggagac |
| 2101 | tcaggactgc tgaaggcacg ggagctgccc ccagtggaca ccaatgaacc ccagtcagcc |
| 2161 | tggaccccta acaaagacca tgaggagatg ctgggaactt tgggactcac ttgattctgc |
| 2221 | agtcgaaata actaatatcc ctacattttt taattaaagc aacagacttc tcaataatca |
| 2281 | atgagttaac cgagaaaact aaaatcagaa gtaagaatgt gctttaaact gaatcacaat |
| 2341 | ataaatatta cacatcacac aatgaaattg aaaaagtaca aaccacaaat gaaaaaagta |
| 2401 | gaaacgaaaa aaaaaaacta ggaaatgaat gacgttggct ttcgtataag gaatttagaa |
| 2461 | aaagaataac caattattcc aaatgaaggt gtaagaaagg gaataagaag aagaagagtt |
| 2521 | gctcatgagg aaaaaccaaa acttgaaaat tcaacaaagc caatgaagct cattcttgaa |
| 2581 | aatattaatt acagtcataa atcctaacta cattgagcaa gagaaagaaa gagcaggcac |
| 2641 | gcatttccat atgggagtga gccagcagac agcccagcag atcctacaca cattttcaca |
| 2701 | aactaacccc agaacaggct gcaaacctat accaatatac tagaaaatgc agattaaatg |
| 2761 | gatgaaatat tcaaaactgg agtttacata atgaacgtaa gagtaatcag agaatctgac |
| 2821 | tcattttaaa tgtgtgtgta tgtgtgtgta tatatatgtg tgtgtgtgtg tgtgtgtgtg |
| 2881 | tgtgtgaaaa acattgactg taataaaaat gttcccatcg taaaaaaaaa aaaaaaa |

SEQ ID NO: 112 Human LILRB2 Isoform Amino Acid Sequence (NP_001074447.2
and NP_001265332.2)

| | |
|---|---|
| 1 | mtpivtvlic lglslgprtr vqtgtipkpt lwaepdsvit qgspvtlscq gsleaqeyrl |
| 61 | yrekksaswi trirpelvkn gqfhipsitw ehtgrygcqy ysrarwsels dplvlvmtga |
| 121 | ypkptlsaqp spvvtsggrv tlqcesqvaf ggfilckege dehpqclnsq phargssrai |
| 181 | fsvgpvspnr rwshrcygyd lnspyvwssp sdllellvpg vskkpslsvq pgpvmapges |
| 241 | ltlqcvsdvg ydrfvlykeg erdlrqlpgr qpqaglsqan ftlgpvsrsy gggyrcyqah |
| 301 | nlssecsaps dpldilitgq irgtpfisvq pgptvasgen vtllcqswrq fhtflltkag |
| 361 | aadaplrlrs iheypkyqae fpmspvtsah agtyrcygsl nsdpyllshp seplelvvsg |
| 421 | psmgsspppt gpistpgped qpltptgsdp qsglgrhlgv vigilvavvl llllllllfl |
| 481 | ilrhrrqgkh wtstqrkadf qhpagavgpe ptdrglqwrs spaadaqeen lyaavkdtqp |
| 541 | edgvemdtra aaseapqdvt yaqlhsltlr rkatepppsq ereppaepsi yatlaih |

SEQ ID NO: 113 Human LILRB2 Transcript Variant 3 cDNA Sequence
(NM_001278403.2 CDS: 129-1922)

| | |
|---|---|
| 1 | ggggaagcca ctgctaccct catcaggaag ggcagacaca agaagcacca gttctatttg |
| 61 | ctgctacatc ccggctctcg caccgagggc tcatccatcc gcagagcagg gcagtgggag |
| 121 | gagacgccat gacccccatc gtcacagtcc tgatctgtct cgggctgagt ctgggcccca |
| 181 | ggacccgcgt gcagacaggg accatcccca agcccaccct gtgggctgag ccagactctg |
| 241 | tgatcaccca ggggagtccc gtcaccctca gttgtcaggg gagccttgaa gcccaggagt |
| 301 | accgtctata tagggagaaa aaatcagcat cttggattac acggatacga ccagagcttg |
| 361 | tgaagaacgg ccagttccac atcccatcca tcacctggga acacacaggg cgatatggct |
| 421 | gtcagtatta cagccgcgct cggtggtctg agctcagtga ccccctggtg ctggtgatga |
| 481 | caggagccta cccaaaaccc accctctcag cccagcccag ccctgtggtg acctcaggag |
| 541 | gaagggtgac cctccagtgt gagtcacagg tggcatttgg cggcttcatt ctgtgtaagg |
| 601 | aaggagaaga tgaacaccca caatgcctga actcccagcc ccatgcccgt gggtcgtccc |
| 661 | gcgccatctt ctccgtgggc cccgtgagcc cgaatcgcag gtggtcgcac aggtgctatg |
| 721 | gttatgactt gaactctccc tatgtgtggt cttcacccag tgatctcctg gagctcctgg |
| 781 | tcccaggtgt ttctaagaag ccatcactct cagtgcagcc gggtcctgtc atggcccctg |
| 841 | gggaaagcct gaccctccag tgtgtctctg atgtcggcta tgacagattt gttctgtaca |
| 901 | aggaggggga acgtgacctt cgccagctcc ctggccggca gccccaggct gggctctccc |
| 961 | aggccaactt caccctgggc cctgtgagcc gctcctacgg gggccagtac agatgctacg |
| 1021 | gtgcacacaa cctctcctct gagtgctcgg cccccagcga ccccctggac atcctgatca |
| 1081 | caggacagat ccgtggcaca cccttcatct cagtgcagcc aggccccaca gtggcctcag |
| 1141 | gagagaacgt gaccctgctg tgtcagtcat ggcggcagtt ccacactttc cttctgacca |
| 1201 | aggcgggagc agctgatgcc ccactccgtc taagatcaat acacgaatat cctaagtacc |
| 1261 | aggctgaatt ccccatgagt cctgtgacct cagcccacgc ggggacctac aggtgctacg |
| 1321 | gctcactcaa ctccgacccc tacctgctgt ctcaccccag tgagcccctg gagctcgtgg |
| 1381 | tctcaggacc ctccatgggt tccagccccc cacccaccgg tcccatctcc acacctggcc |
| 1441 | ctgaggacca gcccctcacc cccactgggt cggatcccca aagtggtctg ggaaggcacc |
| 1501 | tgggggttgt gatcggcatc ttggtggccg tcgtcctact gctcctcctc ctcctcctcc |
| 1561 | tcttcctcat cctccgacat cgacgtcagg gcaaacactg gacatcgacc cagagaaagg |
| 1621 | ctgatttcca acatcctgca ggggctgtgg ggccagagcc cacagacaga ggcctgcagt |
| 1681 | ggaggtccag cccagctgcc gacgcccagg aagaaaacct ctatgctgcc gtgaaggaca |
| 1741 | cacagcctga agatggggtg gagatggaca ctcgggctgc tgcatctgaa gccccccagg |
| 1801 | atgtgaccta cgcccagctg cacagcttga ccctcagacg gaaggcaact gagcctcctc |
| 1861 | catcccagga aagggaacct ccagctgagc ccagcatcta cgccaccctg gccatccact |
| 1921 | agcccggagg gtacgcagac tccacactca gtagaaggag actcaggact gctgaaggca |
| 1981 | cgggagctgc ccccagtgga caccaatgaa ccccagtcag cctggacccc taacaaagac |
| 2041 | catgaggaga tgctgggaac tttgggactc acttgattct gcagtcgaaa taactaatat |
| 2101 | ccctacattt tttaattaaa gcaacagact tctcaataat caatgagtta accgagaaaa |
| 2161 | ctaaaatcag aagtaagaat gtgctttaaa ctgaatcaca atataaatat tacacatcac |
| 2221 | acaatgaaat tgaaaaagta caaaccacaa atgaaaaaag tagaaacgaa aaaaaaaaac |
| 2281 | taggaaatga atgacgttgg ctttcgtata aggaatttag aaaaagaata accaattatt |
| 2341 | ccaaatgaag gtgtaagaaa gggaataaga agaagaagag ttgctcatga ggaaaaacca |
| 2401 | aaacttgaaa attcaacaaa gccaatgaag ctcattcttg aaaatattaa ttacagtcat |
| 2461 | aaatcctaac tacattgagc aagagaaaga aagagcaggc acgcatttcc atatgggagt |
| 2521 | gagccagcag acagcccagc agatcctaca cacattttca caaactaacc ccagaacagg |
| 2581 | ctgcaaacct ataccaatat actagaaaat gcagattaaa tggatgaaat attcaaaact |
| 2641 | ggagtttaca taatgaacgt aagagtaatc agagaatctg actcatttta aatgtgtgtg |
| 2701 | tatgtgtgtg tatatatatg tgtgtgtgtg tgtgtgtgtg tgtgtgtgaa aaacattgac |
| 2761 | tgtaataaaa atgttcccat cgtaaaaaaa aaaaaaaaa |

SEQ ID NO: 114 Human LILRB2 Transcript Variant 4 cDNA Sequence
(NM_001278404.2; CDS: 464-1912)

| | |
|---|---|
| 1 | atttggttga aagaaaaccc acaatccagt gtcaagaaag aagtcaactt ttcttcccct |
| 61 | acttccgtgc atttctcctc tgtgctcact gccacacaca gctcaacctg gacagcacag |
| 121 | ccagaggcga gatgcttctc tgctgatctg agtctgcctg cagcatggac ctgggtcttc |
| 181 | cctgaagcat ctccagggct ggagggacga ctgccatgca ccgagggctc atccatccgc |
| 241 | agagcagggc agtgggagga gacgccatga cccccatcgt cacagtcctg atctgtctcg |
| 301 | ggagaaaaaa tcagcatctt ggattacacg gatacgacca gagcttgtga agaacggcca |
| 361 | gttccacatc ccatccatca cctgggaaca cacagggcga tatggctgtc agtattacag |
| 421 | ccgcgctcgg tggtctgagc tcagtgaccc cctggtgctg gtgatgacag gagcctaccc |
| 481 | aaaacccacc ctatcagccc agcccagccc tgtggtgacc tcaggaggaa gggtgaccct |
| 541 | ccagtgtgag tcacaggtgg catttggcgg cttcattctg tgtaaggaag gagaagatga |
| 601 | acacccacaa tgcctgaact cccagcccca tgcccgtggg tcgtcccgcg ccatcttctc |
| 661 | cgtgggcccc gtgagcccga atcgcaggtg gtcgcacagg tgctatggtt atgacttgaa |
| 721 | ctctccctat gtgtggtctt cacccagtga tctcctggag ctcctggtcc caggtgtttc |
| 781 | taagaagcca tcactctcag tgcagccggg tcctgtcatg gcccctgggg aaagcctgac |
| 841 | cctccagtgt gtctctgatg tcggctatga cagatttgtt ctgtacaagg agggggaacg |
| 901 | tgaccttcgc cagctccctg gccggcagcc ccaggctggg ctctcccagg ccaacttcac |
| 961 | cctgggccct gtgagccgct cctacggggg ccagtacaga tgctacggtg cacacaacct |
| 1021 | ctcctctgag tgctcggccc ccagcgaccc cctggacatc ctgatcacag gacagatccg |
| 1081 | tggcacaccc ttcatctcag tgcagccagg ccccacagtg gcctcaggag agaacgtgac |
| 1141 | cctgctgtgt cagtcatggc ggcagttcca cactttcctt ctgaccaagg cgggagcagc |
| 1201 | tgatgcccca ctccgtctaa gatcaataca cgaatatcct aagtaccagg ctgaattccc |
| 1261 | catgagtcct gtgacctcag cccacgcggg gacctacagg tgctacggct cactcaactc |
| 1321 | cgacccctac ctgctgtctc accccagtga gcccctggag ctcgtggtct caggaccctc |
| 1381 | catgggttcc agccccccac ccaccggtcc catctccaca cctgcaggcc ctgaggacca |
| 1441 | gcccctcacc cccactgggt cggatcccca aagtggtctg ggaaggcacc tgggggttgt |
| 1501 | gatcggcatc ttggtggccg tcgtcctact gctcctcctc ctcctcctcc tcttcctcat |
| 1561 | cctccgacat cgacgtcagg gcaaacactg gacatcgacc cagagaaagg ctgatttcca |
| 1621 | acatcctgca ggggctgtgg ggccagagcc cacagacaga ggcctgcagt ggaggtccag |
| 1681 | cccagctgcc gacgcccagg aagaaaacct ctatgctgcc gtgaaggaca cacagcctga |
| 1741 | agatggggtg gagatggaca ctcgggctgc tgcatctgaa gccccccagg atgtgaccta |
| 1801 | cgcccagctg cacagcttga ccctcagacg gaaggcaact gagcctcctc catcccagga |
| 1861 | aagggaacct ccagctgagc ccagcatcta cgccaccctg gccatccact agcccggagg |
| 1921 | gtacgcagac tccacactca gtagaaggag actcaggact gctgaaggca cgggagctgc |
| 1981 | ccccagtgga caccaatgaa ccccagtcag cctggacccc taacaaagac catgaggaga |
| 2041 | tgctgggaac tttgggactc acttgattct gcagtcgaaa taactaatat ccctacattt |
| 2101 | tttaattaaa gcaacagact tctcaataat caatgagtta accgagaaaa ctaaaatcag |
| 2161 | aagtaaccat gtgctttaaa ctgaatcaca atataaatat tacacatcac acaatgaaat |
| 2221 | tgaaaaagta caaaccacaa atgaaaaaag tagaaacgaa aaaaaaaaac taggaaatga |
| 2281 | atgacgttgg ctttcgtata aggaatttag aaaaagaata accaattatt ccaaatgaag |
| 2341 | gtgtaagaaa gggaataaga agaagaagag ttgctcatga ggaaaaacca aaacttgaaa |
| 2401 | attcaacaaa gccaatgaag ctcattcttg aaaatattaa ttacagtcat aaatcctaac |
| 2461 | tacattgagc aagagaaaga aagagcaggc acgcatttcc atatgggagt gagccagcag |
| 2521 | acagcccagc agatcctaca cacaLtttca caaactaacc ccagaacagg ctgcaaacct |
| 2581 | ataccaatat actagaaaat gcagattaaa tggatgaaat attcaaaact ggagtttaca |
| 2641 | taatgaacgt aagagtaatc agagaatctg actcatttta aatgtgtgtg tatgtgtgtg |
| 2701 | tatatatatg tgtgtgtgtg tgtgtgtgtg tgtgtgtgaa aaacattgac tgtaataaaa |
| 2761 | atgttcccat cgtaaaaaaa aaaaaaaaa |

SEQ ID NO: 115 Human LILRB2 Isoform 3 Amino Acid Sequence
(NP_001265333.2)

| | |
|---|---|
| 1 | mtgaypkptl saqpspvvts ggrvtlqces qvafggfilc kegedehpqc lnsqphargs |
| 61 | sraifsvgpv spnrrwshrc ygvdlnspyv wsspsdllel lvpgvskkps lsvqpgpvma |
| 121 | pgesltlqcv sdvgydrfvl ykegerdlrq lpgrqpqagl sqanftlgpv srsyggqyrc |
| 181 | ygahnlssec sapsdpldil itgqirgtpf isvqpgptva sgenvtllcq swrqfhtfll |
| 241 | tkagaadapl rlrsiheypk yqaefpmspv tsahagtyrc ygslnsdpyl lshpseplel |
| 301 | vvsgpsmgss ppptgpistp agpedqpltp tgsdpqsglg rhlgvvigil vavvllllll |
| 361 | lllflilrhr rqgkhwtstq rkadfghpag avgpeptdrg lqwrsspaad aqeenlyaav |
| 421 | kdtqpedgve mdtraaasea pqdvtyaqlh sltlrrkate pppsqerepp aepsiyatla |
| 481 | ih |

SEQ ID NO: 116 Human LILRB2 Transcript Variant 5 CDNA Sequence
(NM_001278405.2; CDS: 49-1581)

| | |
|---|---|
| 1 | caccgagggc tcatccatcc gcagagcagg gcagtgggag gagacgccat gacccccatc |
| 61 | gtcacagtcc tgatctgtct cgggctgagt ctgggcccca ggacccgcgt gcagacaggg |
| 121 | accatcccca agcccaccct gtgggctgag ccagactctg tgatcaccca ggggagtccc |
| 181 | gtcaccctca gttgtcaggg gagccttgaa gcccaggagt accgtctata tagggagaaa |
| 241 | aaatcagcat cttggattac acggatacga ccagagcttg tgaagaacgg ccagttccac |
| 301 | atcccatcca tcacctggga acacacaggg cgatatggct gtcagtatta cagccgcgct |
| 361 | cggtggtctg agctcagtga ccccctggtg ctggtgatga caggagccta cccaaaaccc |
| 421 | accctatcag cccagcccag ccctgtggtg acctcaggag gaagggtgac cctccagtgt |
| 481 | gagtcacagg tggcatttgg cggcttcatt ctgtgtaagg aaggagaaga tgaacaccca |
| 541 | caatgcctga actcccagcc ccatgcccgt gggtcgtccc gcgccatctt ctccgtgggc |
| 601 | cccgtgagcc cgaatcgcag gtggtcgcac aggtgctatg gttatgactt gaactctccc |
| 661 | tatgtgtggt cttcacccag tgatctcctg gagctcctgg tcccaggtgt ttctaagaag |
| 721 | ccatcactct cagtgcagcc gggtcctgtc atggcccctg gggaaagcct gaccctccag |
| 781 | tgtgtctctg atgtcggcta tgacagattt gttctgtaca aggaggggga acgtgacctt |
| 841 | cgccagctcc ctggccggca gccccaggct gggctctccc aggccaactt caccctgggc |
| 901 | cctgtgagcc gctcctacgg gggccagtac agatgctacg gtgcacacaa cctctcctct |
| 961 | gagtgctcgg cccccagcga ccccctggac atcctgatca caggacagat ccgtggcaca |
| 1021 | cccttcatct cagtgcagcc aggccccaca gtggcctcag gagagaacgt gaccctgctg |
| 1081 | tgtcagtcat ggcggcagtt ccacactttc cttctgacca aggcgggagc agctgatgcc |
| 1141 | ccactccgtc taagatcaat acacgaatat cctaagtacc aggctgaatt ccccatgagt |
| 1201 | cctgtgacct cagcccacgc ggggacctac aggtgctacg gctcactcaa ctccgacccc |
| 1261 | tacctgctgt ctcaccccag tgagcccctg gagctcgtgg tctcaggacc ctccatgggt |
| 1321 | tccagccccc cacccaccgg tcccatctcc acacctgcag gccctgagga ccagcccctc |
| 1381 | acccccactg ggtcggatcc ccaaagtggt ctgggaaggc acctgggggt tgtgatcggc |
| 1441 | atcttggtgg ccgtcgtcct actgctcctc ctcctcctcc tcctcttcct catcctccga |
| 1501 | catcgacgtc agggcaaaca ctggacatcg agtccagccc agctgccgac gcccaggaag |
| 1561 | aaaacctcta tgctgccgtg aaggacacac agcctgaaga tggggtggag atggacactc |
| 1621 | gggctgctgc atctgaagcc ccccaggatg tgacctacgc ccagctgcac agcttgaccc |
| 1681 | tcagacggaa ggcaactgag cctcctccat cccagggaag ggaacctcca gctgagccca |
| 1741 | gcatctacgc caccctggcc atccactagc ccggagggta cgcagactcc acactcagta |
| 1801 | gaaggagact caggactgct gaaggcacgg gagctgcccc cagtggacac caatgaaccc |
| 1861 | cagtcagcct ggacccctaa caaagaccat gaggagatgc tgggaacttt gggactcact |
| 1921 | tgattctgca gtcgaaataa ctaatatccc tacatttttt aattaaagca acagacttct |
| 1981 | caataatcaa tgagttaacc gagaaaacta aaatcagaag taagaatgtg ctttaaactg |
| 2041 | aatcacaata taaatattac acatcacaca atgaaattga aaaagtacaa accacaaatg |
| 2101 | aaaaaagtag aaacgaaaaa aaaaaactag gaaatgaatg acgttggctt tcgtataagg |
| 2161 | aatttagaaa aagaataacc aattattcca aatgaaggtg taagaaaggg aataagaaga |
| 2221 | agaagagttg ctcatgagga aaaaccaaaa cttgaaaatt caacaaagcc aatgaagctc |
| 2281 | attcttgaaa atattaatta cagtcataaa tcctaactac attgagcaag agaaagaaag |
| 2341 | agcaggcacg catttccata tgggagtgag ccagcagaca gcccagcaga tcctacacac |
| 2401 | attttcacaa actaacccca gaacaggctg caaacctata ccaatatact agaaaatgca |
| 2461 | gattaaatgg atgaaatatt caaaactgga gtttarataa tgaacgtaag agtaatcaga |
| 2521 | gaatctgact cattttaaat gtgtgtgtat gtgtgtgtat atatatgtgt gtgtgtgtgt |
| 2581 | gtgtgtgtgt gtgtgaaaaa cattgactgt aataaaaatg ttcccatcgt aaaaaaaaaa |
| 2641 | aaaaaa |

SEQ ID NO: 117 Human LILRB2 Isoform 4 Amino Acid Sequence
(NP_001265334.2)

| | |
|---|---|
| 1 | mtpivtvlic lglslgprtr vqtgtipkpt lwaepdsvit qgspvtlscq gsleaqevrl |
| 61 | yrekksaswi trirpelvkn gqfhipsitw ehtgrygcqy ysrarwsels dplvlvmtga |
| 121 | ypkptlsaqp spvvtsggrv tlqcesqvaf qgfilckege dehpqclnsq phargssrai |
| 181 | fsvgpvspnr rwshrcygyd lnspyvwssp sdllellvpg vskkpslsvq pgpvmapges |
| 241 | ltlqcvsdvg ydrfvlvkeg erdlrqlpgr qpqaglsqan ftlgpvsrsy ggqyrcygah |
| 301 | nlssecsaps dpldilitgq irgtpfisvg pgptvasgen vtllcqswrq fhtflltkag |
| 361 | aadaplrlrs iheypkyqae fpmspvtsah agtyrcygsl nsdpyllshp seplelvvsg |
| 421 | psmgsspppt gpistpagpe dqpltptgsd pqsglgrhlg vvigilvavv lllllllllf |
| 481 | lilrhrrqgk hwtsspaqlp tprkktsmlp |

SEQ ID NO: 118 Human LILRB2 Transcript Variant 6 CDNA Sequence
(NM_001278406.2; CDS: 49-1416)

| | |
|---|---|
| 1 | caccgagggc tcatccatcc gcagagcagg gcagtgggag gagacgccat gacccccatc |
| 61 | gtcacagtcc tgatctgtct cgggctgagt ctgggcccca ggacccgcgt gcagacaggg |
| 121 | accatcccca agcccaccct gtgggctgag ccagactctg tgatcaccca ggggagtccc |
| 181 | gtcaccctca gttgtcaggg gagccttgaa gcccaggagt accgtctata tagggagaaa |
| 241 | aaatcagcat cttggattac acggatacga ccagagcttg tgaagaacgg ccagttccac |
| 301 | atcccatcca tcacctggga acacacaggg cgatatggct gtcagtatta cagccgcgct |
| 361 | cggtggtctg agctcagtga ccccgtggtg ctggtgatga caggagccta cccaaaaccc |
| 421 | accctctcag cccagcccag ccctgtggtg acctcaggag gaagggtgac cctccagtgt |
| 481 | gagtcacagg tggcatttgg cggcttcatt ctgtgtaagg aaggagaaga tgaacaccca |
| 541 | caatgcctga actcccagcc ccatgcccgt gggtcgtccc gcgccatctt ctccgtgggc |
| 601 | cccgtgagcc cgaatcgcag gtggtcgcac aggtgctatg gttatgactt gaactctccc |
| 661 | tatgtgtggt cttcacccag tgatctcctg gagctcctgg tcccaggtgt ttctaagaag |
| 721 | ccatcactct cagtgcagcc gggtcctgtc atggcccctg gggaaagcct gaccctccag |
| 781 | tgtgtctctg atgtcggcta tgacagattt gttctgtaca aggaggggga acgtgacctt |
| 841 | cgccagctcc ctggccggca gccccaggct gggctctccc aggccaactt caccctgggc |
| 901 | cctgtgagcc gctcctacgg gggccagtac agatgctacg gtgcacacaa cctctcctct |
| 961 | gagtgctcgg cccccagcga ccccctggac atcctgatca caggacagat ccgtggcaca |
| 1021 | cccttcatct cagtgcagcc aggccccaca gtggcctcag gagagaacgt gaccctgctg |
| 1081 | tgtcagtcat ggcggcagtt ccacactttc cttctgacca aggcgggagc agctgatgcc |
| 1141 | ccactccgtc taagatcaat acacgaatat cctaagtacc aggctgaatt ccccatgagt |
| 1201 | cctgtgacct cagcccacgc ggggacctac aggtgctacg gctcactcaa ctccgacccc |
| 1261 | tacctgctgt ctcaccccag tgagcccctg gagctcgtgg tctcaggacc ctccatgggt |
| 1321 | tccagccccc cacccaccgg tcccatctcc acacctgcag gccctgagga ccagcccctc |
| 1381 | acccccactg ggtcggatcc ccaaagtggt gagtgagggg ct |

SEQ ID NO: 119 Human LILRB2 Isoform 5 Amino Acid Sequence
(NP_001265335.2)

| | |
|---|---|
| 1 | mtpivtvlic lglslgprtr vqtgtipkpt lwaepdsvit qgspvtlscq gsleaqeyrl |
| 61 | yrekksaswi trirpelvkn gqfhipsitw ehtgrygcqy ysrarwsels dplvlvmtga |
| 121 | ypkptlsaqp spvvtsggrv tlqcesqvaf ggfilckege dehpqclnsq phargssrai |
| 181 | fsvgpvspnr rwshrcygyd lnspyvwssp sdllellvpg vskkpslsvq pgpvmapges |
| 241 | ltlqcvsdvg ydrfvlykeg erdlrqlpgr qpqaglsqan ftlgpvsrsy ggqyrcygah |
| 301 | nlssecsaps dpldilitgq irgtpfisvq pgptvasgen vtllcqswrq fhtflltkag |
| 361 | aadaplrlrs iheypkyqae fpmspvtsah agtyrcygsl nsdpyllshp seplelvvsg |
| 421 | psmgsspppt gpistpagpe dqpltptgsd pqsge |

SEQ ID NO: 120 Human CCR5 Transcript Variant A cDNA Sequence
(NM_000579.3; CDS: 358-1416

| | |
|---|---|
| 1 | cttcagatag attatatctg gagtgaagaa tcctgccacc tatgtatctg gcatagtatt |
| 61 | ctgtgtagtg ggatgagcag agaacaaaaa caaaataatc cagtgagaaa agcccgtaaa |
| 121 | taaaccttca gaccagagat ctattctcta gcttatttta agctcaactt aaaaagaaga |
| 181 | actgttctct gattcttttc gccttcaata cacttaatga tttaactcca ccctccttca |
| 241 | aaagaaacag catttcctac ttttatactg tctatatgat tgatttgcac agctcatctg |
| 301 | gccagaagag ctgagacatc cgttccccta caagaaactc tccccgggtg gaacaagatg |
| 361 | gattatcaag tgtcaagtcc aatctatgac atcaattatt atacatcgga gccctgccaa |
| 421 | aaaatcaatg tgaagcaaat cgcagcccgc ctcctgcctc cgctctactc actggtgttc |
| 481 | atctttggtt ttgtgggcaa catgctggtc atcctcatcc tgataaactg caaaaggctg |
| 541 | aagagcatga ctgacatcta cctgctcaac ctggccatct ctgacctgtt tttccttctt |
| 601 | actgtcccct tctgggctca ctatgctgcc gcccagtggg actttggaaa tacaatgtgt |
| 661 | caactcttga cagggctcta ttttataggc ttcttctctg gaatcttctt catcatcctc |
| 721 | ctgacaatcg ataggtacct ggctgtcgtc catgctgtgt ttgctttaaa agccaggacg |
| 781 | gtcacctttg gggtggtgac aagtgtgatc acttgggtgg tggctgtgtt tgcgtctctc |
| 841 | ccaggaatca tctttaccag atctcaaaaa gaaggtcttc attacacctg cagctctcat |
| 901 | tttccataca gtcagtatca attctggaag aatttccaga cattaaagat agtcatcttg |
| 961 | gggctggtcc tgccgctgct tgtcatggtc atctgctact cgggaatcct aaaaactctg |
| 1021 | cttcggtgtc gaaatgagaa gaagaggcac agggctgtga ggcttatctt caccatcatg |
| 1081 | attgtttatt ttctcttctg ggctccctac aacattgtcc ttctcctgaa caccttccag |
| 1141 | gaattctttg gcctgaataa ttgcagtagc tctaacaggt tggaccaagc tatgcaggtg |
| 1201 | acagagactc ttgggatgac gcactgctgc atcaacccca tcatctatgc ctttgtcggg |
| 1261 | gagaagttca gaaactacct cttagtcttc ttccaaaagc acattgccaa acgcttctgc |
| 1321 | aaatgctgtt ctattttcca gcaagaggct cccgagcgag caagctcagt ttacacccga |
| 1381 | tccactgggg agcaggaaat atctgtgggc ttgtgacacg gactcaagtg ggctggtgac |
| 1441 | ccagtcagag ttgtgcacat ggcttagttt tcatacacag cctgggctgg gggtggggtg |
| 1501 | gccgaggtct tttttaaaag gaagttactg ttatagaggg tctaagattc atccatttat |
| 1561 | ttggcatctg tttaaagtag attagatctt ttaagcccat caattataga aagccaaatc |
| 1621 | aaaatatgtt gatgaaaaat agcaaccttt ttatctcccc ttcacatgca tcaagttatt |
| 1681 | gacaaactct cccttcactc cgaaagttcc ttatgtatat ttaaaagaaa gcctcagaga |
| 1741 | attgctgatt cttgagttta gtgatctgaa cagaaatacc aaaattattt cagaaatgta |
| 1801 | caacttttta cctagtacaa ggcaacatat aggttgtaaa tgtgtttaaa acaggtcttt |
| 1861 | gtcttgctat ggggagaaaa gacatgaata tgattagtaa agaaatgaca cttttcatgt |
| 1921 | gtgatttccc ctccaaggta tggttaataa gtttcactga cttagaacca ggcgagagac |
| 1981 | ttgtggcctg ggagagctgg ggaagcttct taaatgagaa ggaatttgag ttggatcatc |
| 2041 | tattgctggc aaagacagaa gcctcactgc aagcactgca tgggcaagct tggctgtaga |
| 2101 | aggagacaga gctggttggg aagacatggg gaggaaggac aaggctagat catgaagaac |
| 2161 | cttgacggca ttgctccgtc taagtcatga gctgagcagg gagatcctgg ttggtgttgc |
| 2221 | agaaggttta ctctgtggcc aaaggagggt caggaaggat gagcatttag ggcaaggaga |
| 2281 | ccaccaacag ccctcaggtc agggtgagga tggcctctgc taagctcaag gcgtgaggat |
| 2341 | gggaaggagg gaggtattcg taaggatggg aaggagggag gtattcgtgc agcatatgag |
| 2401 | gatgcagagt cagcagaact ggggtggatt tgggttggaa gtgagggtca gagaggagtc |
| 2461 | agagagaatc cctagtcttc aagcagattg gagaaaccct tgaaaagaca tcaagcacag |
| 2521 | aaggaggagg aggaggttta ggtcaagaag aagatggatt ggtgtaaaag gatgggtctg |
| 2581 | gtttgcagag cttgaacaca gtctcaccca gactccaggc tgtctttcac tgaatgcttc |
| 2641 | tgacttcata gatttccttc ccatcccagc tgaaatactg aggggtctcc aggaggagac |
| 2701 | tagatttatg aatacacgag gtatgaggtc taggaacata cttcagctca cacatgagat |
| 2761 | ctaggtgagg attgattacc tagtagtcat ttcatgggtt gttgggagga ttctatgagg |
| 2821 | caaccacagg cagcatttag cacatactac acattcaata agcatcaaac tcttagttac |
| 2881 | tcattcaggg atagcactga gcaaagcatt gagcaaaggg gtcccataga ggtgagggaa |
| 2941 | gcctgaaaaa ctaagatgct gcctgcccag tgcacacaag tgtaggtatc attttctgca |
| 3001 | tttaaccgtc aataggcaaa ggggggaagg gacatattca tttggaaata agctgccttg |
| 3061 | agccttaaaa cccacaaaag tacaatttac cagcctccgt atttcagact gaatgggggt |
| 3121 | ggggggggcg ccttaggtac ttattccaga tgccttctcc agacaaacca gaagcaacag |
| 3181 | aaaaaatcgt ctctccctcc ctttgaaatg aatatacccc ttagtgtttg ggtatattca |
| 3241 | tttcaaaggg agagagagag gtttttttct gttctgtctc atatgattgt gcacatactt |
| 3301 | gagactgttt tgaatttggg ggatggctaa aaccatcata gtacaggtaa ggtgagggaa |
| 3361 | tagtaagtgg tgagaactac tcagggaatg aaggtgtcag aataataaga ggtgctactg |
| 3421 | actttctcag cctctgaata tgaacggtga gcattgtggc tgtcagcagg aagcaacgaa |
| 3481 | gggaaatgtc tttccttttg ctcttaagtt gtggagagtg caacagtagc ataggaccct |
| 3541 | accctctggg ccaagtcaaa gacattctga catcttagta tttgcatatt cttatgtatg |
| 3601 | tgaaagttac aaattgcttg aaagaaaata tgcatctaat aaaaaacacc ttctaaaata |
| 3661 | aaaaaaaaaa aaaaaaaaaa aaaaaa |

SEQ ID NO: 121 Human CCR5 Transcript Variant B cDNA Sequence
(NM_001100168.1; CDS: 123-1181)

| | |
|---|---|
| 1 | cttcagatag attatatctg gagtgaagaa tcctgccacc tatgtatctg gcatagtctc |
| 61 | atctggccag aagagctgag acatccgttc ccctacaaga aactctcccc gggtggaaca |
| 121 | agatggatta tcaagtgtca agtccaatct atgacatcaa ttattataca tcggagccct |
| 181 | gccaaaaaat caatgtgaag caaatcgcag cccgcctcct gcctccgctc tactcactgg |
| 241 | tgttcatctt tggttttgtg ggcaacatgc tggtcatcct catcctgata aactgcaaaa |
| 301 | ggctgaagag catgactgac atctacctgc tcaacctggc catctctgac ctgtttttcc |
| 361 | ttcttactgt ccccttctgg gctcactatg ctgccgccca gtgggacttt ggaaatacaa |
| 421 | tgtgtcaact cttgacaggg ctctatttta taggcttctt ctctggaatc ttcttcatca |
| 481 | tcctcctgac aatcgatagg tacctggctg tcgtccatgc tgtgtttgct ttaaaagcca |
| 541 | ggacggtcac ctttggggtg gtgacaagtg tgatcacttg ggtggtggct gtgtttgcgt |
| 601 | ctctcccagg aatcatcttt accagatctc aaaaagaagg tcttcattac acctgcagct |
| 661 | ctcattttcc atacagtcag tatcaattct ggaagaattt ccagacatta aagatagtca |
| 721 | tcttggggct ggtcctgccg ctgcttgtca tggtcatctg ctactcggga atcctaaaaa |
| 781 | ctctgcttcg gtgtcgaaat gagaagaaga ggcacagggc tgtgaggctt atcttcacca |
| 841 | tcatgattgt ttattttctc ttctgggctc cctacaacat tgtccttctc ctgaacacct |
| 901 | tccaggaatt ctttggcctg aataattgca gtagctctaa caggttggac caagctatgc |
| 961 | aggtgacaga gactcttggg atgacgcact gctgcatcaa ccccatcatc tatgcctttg |
| 1021 | tcggggagaa gttcagaaac tacctcttag tcttcttcca aaagcacatt gccaaacgct |
| 1081 | tctgcaaatg ctgttctatt ttccagcaag aggctcccga gcgagcaagc tcagtttaca |
| 1141 | cccgatccac tggggagcag gaaatatctg tgggcttgtg acacggactc aagtgggctg |
| 1201 | gtgacccagt cagagttgtg cacatggctt agttttcata cacagcctgg gctgggggtg |
| 1261 | gggtgggaga ggtctttttt aaaaggaagt tactgttata gagggtctaa gattcatcca |
| 1321 | tttatttggc atctgtttaa agtagattag atcttttaag cccatcaatt atagaaagcc |
| 1381 | aaatcaaaat atgttgatga aaaatagcaa cctttttatc tccccttcac atgcatcaag |
| 1441 | ttattgacaa actctccctt cactgrgaaa gttccttatg tatatttaaa agaaagcctc |
| 1501 | agagaattgc tgattcttga gtttagtgat ctgaacagaa ataccaaaat tatttcagaa |
| 1561 | atgtacaact ttttacctag tacaaggcaa catataggtt gtaaatgtgt ttaaaacagg |
| 1621 | tctttgtctt gctatgggga gaaaagacat gaatatgatt agtaaagaaa tgacactttt |
| 1681 | catgtgtgat ttcccctcca aggtatggtt aataagtttc actgacttag aaccaggcga |
| 1741 | gagacttgtg gcctgggaga gctggggaag cttcttaaat gagaagccat ttgagttgga |
| 1801 | tcatctattg ctggcaaaga cagaagcctc actgcaagca ctgcatgggc aagcttggct |
| 1861 | gtagaaggag acagagctgg ttgggaagac atggggagga aggacaaggc tagatcatga |
| 1921 | agaaccttga cggcattgct ccgtctaagt catgagctga gcagggagat cctggttgta |
| 1981 | gttgcagaag gtttactctg tggccaaagg agggtcagga aggatgagca tttagggcaa |
| 2041 | ggagaccacc aacagccctc aggtcagggt gaggatggcc tctgctaagc tcaaggcgtg |
| 2101 | aggatgggaa ggagggaggt attcgtaagc atgggaagga gggaggtatt cgtgcagcat |
| 2161 | atgaggatgc agagtcagca gaaactgggg ggatttgggt tggaagtgag ggtcagagag |
| 2221 | gagtcagaga gaatccctag tcttcaagca gattggagaa acccttgaaa agacatcaag |
| 2281 | cacagaagga ggaggaggag gtttaggtca agaagaagat ggattggtgt aaaaggatgg |
| 2341 | gtctggtttg cagagcttga acacagtctc acccagactc caggctgtct ttcactgaat |
| 2401 | gcttctgact tcatagattt ccttcccatc ccagctgaaa tactgagggg tctccaggag |
| 2461 | gagactagat ttatgaatac acgaggtatg aggtctagga acatacttca gctcacacat |
| 2521 | gagatctagg tgaggattga ttacctagta gtcatttcat gggttgttgg gaggattcta |
| 2581 | tgaggcaacc acaggcagca tttagcacat actacacatt caataagcat caaactctta |
| 2641 | gttactcatt cagggatagc actgagcaaa gcattgagca aaggggtccc atagaggtga |
| 2701 | gggaagcctg aaaaactaag atgctgcctg cccagtgcac acaagtgtag gtatcatttt |
| 2761 | ctgcatttaa ccgtcaatag gcaaaggggg gaagggacat attcatttgg aaataagctg |
| 2821 | ccttgagcct taaaacccac aaaagtacaa tttaccagcc tccgtatttc agactgaatg |
| 2881 | ggggtggggg gggcgcctta ggtacttatt ccagatgcct tctccagaca aaccagaagc |
| 2941 | aacagaaaaa atcgtctctc cctccctttg aaatgaatat accccttagt gtttgggtat |
| 3001 | attcatttca aagggagaga gagaggtttt tttctgttct gtctcatatg attgtgcaca |
| 3061 | tacttgagac tgttttgaat ttgggggatg gctaaaacca tcatagtaca ggtaaggtga |
| 3121 | gggaatagta agtggtgaga actactcagg gaatgaaggt gtcagaataa taagaggtgc |
| 3181 | tactgacttt ctcagcctct gaatatgaac ggtgagcatt gtggctgtca gcaggaagca |
| 3241 | acgaagggaa atgtctttcc tattgctctt aagttgtgga gagtgcaaca gtagcatagg |
| 3301 | accctaccct ctgggccaag tcaaagacat tctgacatct tagtatttgc atattcttat |
| 3361 | gtatgtgaaa gttacaaatt gcttgaaaga aaatatgcat ctaataaaaa acaccttcta |
| 3421 | aaataaaaaa aaaaaaaaaa aaaaaaaaaa a |

SEQ ID NO: 122 Human CCR5 Amino Acid Sequence (NP_000570.1 and
NP_001093638.1)

| | |
|---|---|
| 1 | mdyqvsspiy dinyytsepc qkinvkqiaa rllpplyslv fifgfvgnml vililinckr |
| 61 | lksmtdlyll nlaisdlffl ltvpfwahya aaqwdfgntm cqlltglyfi gffsgiffii |
| 121 | lltidrylav vhavfalkar tvtfgvvtsv itwvvavfas lpgiiftrsq keglhytcss |
| 181 | hfpysqyqfw knfqtlkivi lglvlpllvm vicvsgilkt llrcrnekkr hravrlifti |
| 241 | mivyflfwap ynivlllntf qeffglnncs ssnrldqamq vtetlgmthc cinpiiyafv |
| 301 | gekfrnyllv ffqkhiakrf ckccsifqqe aperassvyt rstgeqeisv gl |

SEQ ID NO: 123 Human EVI2B cDNA Sequence (NM_006495.3; CDS: 156-1502)

| | |
|---|---|
| 1 | agaaactatt tcagtttgct agaggaacat tttaaatctg aattgaacca cccattttcc |
| 61 | tttcttagcc aaatcaccaa aatgtccagt tagaacaaga atttagcatt ctgcaaaaga |
| 121 | agttaacagc tgagataacg aggaaatatt ctgaaatgga tcccaaatat ttcatcttaa |
| 181 | ttttgttttg tggacacctg aacaatacat ttttttcaaa gacagagaca attacaacag |
| 241 | agaagcagtc acagcctacc ttattcacat catcaatgtc acaggtattg gctaattctc |
| 301 | aaaacacaac agggaatcct ttgggtcaac caacacaatt cagcgacact ttttctggac |
| 361 | aatcaatatc acctgccaaa gtcactgctg gacaaccaac accagctgtc tatacctctt |
| 421 | ctgaaaaacc agaagcacat acttctgctg gacaaccact tgcctacaac accaaacaac |
| 481 | caacaccaat agccaacacc tcctcccagc aagccgtgtt cacctctgcc agacaactac |
| 541 | catctgcccg tacttctacc acacaaccac caaagtcatt tgtctatact tttactcaac |
| 601 | aatcatcatc tgtccagatc ccttctagaa aacaaataac tgttcataat ccatccacac |
| 661 | aaccaacatc aactgtcaaa aattcaccta ggagtacacc aggatttatc ttagatacta |
| 721 | ccagtaacaa acaaacccca caaaaaaaca attataattc aatagctgcc atactaattg |
| 781 | gtgtacttct gacttctatg ttggtagcta taatcatcat tgtactttgg aaatgcttaa |
| 841 | ggaaaccagt tttaaatgat caaaattggg caggtagatc tccatttgct gatggagaaa |
| 901 | cccctgacat ttgtatggat aacatcagag aaaatgaaat atccacaaaa cgtacatcaa |
| 961 | tcatttcact tacaccctgg aaaccaagca aaagcacact tttagcagat gacttagaaa |
| 1021 | ttaagttgtt tgaatcaagt gaaaacattg aagactccaa caaccccaaa acagagaaaa |
| 1081 | taaaagatca agtaaatggt acatcagaag atagtgctga tggttcaaca gttggaactg |
| 1141 | ctgtttcttc ttcagatgat gcagatctgc ctccaccacc tccccttctg gatttggaag |
| 1201 | gacaggaaag taaccaatct gacaaaccca caatgacaat tgtatctcct cttccaaatg |
| 1261 | attctactag tctccctcca tctctggact gtctcaatca agactgtgga gatcataaat |
| 1321 | ctgagataat acaatcattt ccaccgcttg actcacttaa cttgcccctg ccaccagtag |
| 1381 | attttatgaa aaaccaagaa gattccaacc ttgagatcca gtgtcaggag ttctctattc |
| 1441 | ctcccaactc tgatcaagat cttaatgaat ccctgccacc tccacctgca gaactgttat |
| 1501 | aaatattaca acttgctttt tagctgatct tccatcctca aatgactctt ttttctttat |
| 1561 | atgttaacat atataaaatg gcaactgata gtcaattttg atttttattc aggaactatc |
| 1621 | tgaaatctgc tcagagccta tgtgcataga tgaaactttt ttttaaaaaa agttatttaa |
| 1681 | cagtaatcta tttactaatt atagtaccta tctttaaagt atagtacatt ttacatatgt |
| 1741 | aaatggtatg tttcaataat ttaagaactc tgaaacaatc tacatatact tattacccag |
| 1801 | tacagttttt tttcccctga aaagctgtgt ataaaattat ggtgaataaa cttttatgtt |
| 1861 | tccatttcaa agaccagggt ggagaggaat aagagactaa gtatatgctt caagttttaa |
| 1921 | attaatacct caagtattaa ataaatattc caagtttgtg ggaatgggag attaaaatgc |
| 1981 | atgtttgaga atagaaaaaa aaaaaaaaa |

SEQ ID NO: 124 Human EVI2B Amino Acid Sequence (NP_006486.3)

| | |
|---|---|
| 1 | mdpkyfilil fcghlnntff sktetittek qsqptlftss msqvlansqn ttgnplgqpt |
| 61 | qfsdtfsgqs ispakvtagq ptpavytsse kpeahtsagq playntkqpt piantssqqa |
| 121 | vftsarqlps artsttqppk sfvytftqqs ssvqipsrkq itvhnpstqp tstvknsprs |
| 181 | tpgfildtts nkqtpqknny nsiaailigv lltsmlvaii iivlwkclrk pvlndqnwag |
| 241 | rspfadgetp dicmdniren eistkrtsii sltpwkpsks tlladdleik lfessenied |
| 301 | snnpktekik dqvngtseds adgstvgtav sssddadlpp ppplldlegq esnqsdkptm |
| 361 | tivsplpnds tslppsldcl nqdcgdhkse iiqsfpplds lnlplppvdf mknqedsnle |
| 421 | iqcqefsipp nsdqdlnesl ppppaell |

SEQ ID NO: 125 Mouse EVI2B cDNA Sequence (NM_001077496.1; CDS: 167-1501)

| | |
|---|---|
| 1 | tactgataaa cttcctctgc tgcacagaag ccgtctcagt gcgctggaga ccaccgttaa |
| 61 | tccgcactga aacacccctt tgctctctca gccaaatcac caacatgtcc tgttagaaca |
| 121 | agaatttaca ttctgcgaaa gaagttcaca ggagaaaaca tctgaaatgg aattcaagta |
| 181 | tctggtcttc attgtgcttt gtcaatacct ggacaatacg tttttctcag agacagaagc |
| 241 | aattacaaca gagcagcaat cactgtctac tttaatcaca ccgtcgttat atgttacaac |
| 301 | tgattctcaa aacacagcag ggaatgcttt gagtcagaca acaagattca agaacatttc |
| 361 | ttctggacag caagcatcac ctgcccaaat cactcctgaa caagcaacac cagctgttta |
| 421 | tgtctcttca agcccactta cttataacat taccagacaa gcagaatcag cggtcaacaa |
| 481 | ctccttgcct caaacatcac catctgggtt cactttgacc aatcagccat caccttctac |
| 541 | ctataattct actggacaac caccaaaaca tcttgtctat acttccacac aacagccacc |
| 601 | atcacctgct cctacctctt ctggaaaacc agaagtagag tctactcata atcagcccac |
| 661 | aaaatcaaca ccaactattt atttacaaag ggacacacca ccaccaccac caccaccact |
| 721 | cacttcagaa ccaccaagtg gcaaaggaac tgctcataaa aacaaccaca atgcaattgc |
| 781 | tgccatatta atcggtacta ttataatttc tatgttggta gctatactca tgattatact |
| 841 | gtggaaatac ttgaggaagc cagtcttaaa tgatcaaaac tgggcaggta ggtctccatt |
| 901 | tgctgatgga gaaacaccag aaatgtgtat ggataacatc agagagagtg aggcatccac |
| 961 | aaagcgtgca tcagttgtct cacttatgac ctggaaacca agtaaaagca cactgctagc |
| 1021 | agatgattta gaagttaagt tgtttgaatc aagtgaacac attaatgata ccagcaacct |
| 1081 | caagactgat aatgtagaag ttcaaataaa tggcttatca gaagacagtg ctgatgggtc |
| 1141 | aacagttggt acagccgtgt cttcagatga tgcagatctg gccctgccac ctccccttct |
| 1201 | tgatttggat gagaacctac caaacaagcc cacagtgaca gttgtatctc ctttaccaaa |
| 1261 | tgattctatc aatccccaac catctccaga tggtctaaat caagtgtgtg aagaacagca |
| 1321 | ttccaagatc caagagccat tcccgccacc ccctgactca tttaatgtgc ctctgtcagc |
| 1381 | aggagatttt ataaacaatc aagagtcggc ccacgaggct caatgccagg agttctctac |
| 1441 | tcctgacctc catccagacc tcacagactc cctgccacct ccacctacag agctgctgta |
| 1501 | aagtgacagc tggctttcta gactgccctc catcctgaga tggcaccgac acgttcctgt |
| 1561 | cttcgtggta tggcacacag gacatgacac ctggcacctg agagactctt tagggactac |
| 1621 | ctgacccctg actgaagccc acgtacacgg gtagattatt acagtggtat gcttaccagc |
| 1681 | tacagaagta atttttaaat cagagtgcct tttatatatg ttaatgatag atatttcaat |
| 1741 | aacttaatca ctttggaaac atatgatcta catatactta ttattaccca atagatttcc |
| 1801 | cccgaaaagc tgtatataaa catatcttaa ataaataaaa ttctattttt tccacctcaa |
| 1861 | aatttggggt ataataaaaa tggaaatttt taaatggtca aagttttaat acttcaaata |
| 1921 | ttaagtaaat attttcattt atagagatta aacacacagc taccaaactg tattaaaaca |
| 1981 | tgaagtctga gtgctgtgtt tgggatacat ttaccctgaa aaagtgagca cttaccataa |
| 2041 | aattttcttt tttctttata attaaaaatt ttatgtatat aagagttttg cctgaatatg |
| 2101 | tgtgtatata ttcatatagt cacacacaca cacatatata taagacagaa gatggcacta |
| 2161 | ctaacatttg ccaatgcccc gtgagtacct gaactgactc caagtcctct gtaagtgctc |
| 2221 | ttaaccaccg agcccctgca actccgttct tcccctcctc ttcctcctcc tcctcttcct |
| 2281 | cctcctcttc ctcttcaaat gtttggggcg tgggggtgga ggtcaaagag ataggctgta |
| 2341 | tttggggtta gaagatgact tgtgggagtt gatttttttc ttccacctca aggataaaat |
| 2401 | tcagaactca agtcatgaga cctgacagca agcaaccttt atcctctaag cccatctcac |
| 2461 | tgcccccaaa atgtaaacat ttttctaaaa tgttagggaa gcataaacta accacttaga |
| 2521 | caaaaatcaa atgatatgga agacgggaga aaaaaataga agtgggctgt atctgtgtgt |
| 2581 | ctcttcccac acatgcatga taccagcctg tgtgagggtg tctgtggaga ccacagagga |
| 2641 | taccagccat cctggaactg aagtcccagg aagttatgag ctgctgtcta acacgggtac |
| 2701 | tgggaactga acttgggtcc tataggacag cagcaagttc tctaaagtac tgacccatca |
| 2761 | ctgcagcccc agcctccttt tgtaaatgca ggaatgtaat catcacctga aggttgttga |
| 2821 | gtatgaggca ctcagaacag aagtgggcac aacttttgtt aaagtggagg cggcagtggg |
| 2881 | tactggccta ccttttgcag atgtcaaatg ttcatctgcc caacaagcca tccttctctt |
| 2941 | attattatta caagtccaag aaaggacagt tgcttagagc acagattttg gcacttttca |
| 3001 | agatagtata tagtatttat tggtaacatt aatgttcaaa tactaaaaat cccatgtaga |
| 3061 | ataaaaacaa gaaggttctc gggcagtgga gttttactac tactgctact attactacta |
| 3121 | catactacta ctattattta gtatataatt atcaataaac aattattata catattataa |
| 3181 | atacatgtaa atttattatt gttgttgttt gtgttctgag acaggagtcc tctggtagcc |
| 3241 | ctggctgtcc tggcactctc tctctataga cccactgtcc tcaaactcac agatacacct |
| 3301 | gccfactgtc cccaatcttt gtgattaacg gtgtgacctt taaacacctt aaaagacact |
| 3361 | gggcttaagt gttattttac atacaatact taccttactt cttacaagcc acctcttact |
| 3421 | gataacactt tttttatcta cagaaacacc tacttcaagc atgatggcgt ttcattttat |
| 3481 | tacattggtt cttttctgtc attcacaata aaatttctca cgtgtcttc |

SEQ ID NO: 126 Mouse EVI2B Amino Acid Sequence (NP_001070964.1)

| | |
|---|---|
| 1 | mefkylvfiv lcqyldntff seteaitteq qslstlitps lyvttdsqnt agnalsqttr |
| 61 | fknissgqqa spaqitpeqa tpavyvsssp ltynitrqae savnnslpqt spsgftltnq |
| 121 | pspstynstg qppkhlvyts tqqppspapt ssgkpevest hnqptkstpt iylqrdtppp |
| 181 | ppppltsepp sgkgtahknn hnaiaailig tiiismlvai lmiilwkylr kpvindqnwa |
| 241 | grspfadget pemcmdnire seastkrasv vslmtwkpsk stlladdlev klfessehin |
| 301 | dtsnlktdnv evqinglsed sadgstvgta vssddadlal ppplldlden lpnkptvtvv |
| 361 | splpndsinp qpspdglnqv ceeqhskiqe pfppppdsfn vplsagdfin nqesaheaqc |
| 421 | qefstpdlhp dltdslpppp tell |

SEQ ID NO: 127 Human CLEC7A Transcript Variant 1 cDNA Sequence
(NM_197947.2; CDS: 188-931)

| | |
|---|---|
| 1 | agcatagttt catttcctgc tcttgaatat ctggttgaac tacttaagct taatttgtta |
| 61 | aactccggta agtacctagc ccacatgatt tgactcagag attctctttt gtccacagac |
| 121 | agtcatctca ggagcagaaa gaaaagagct cccaaatgct atatctattc aggggctctc |
| 181 | aagaacaatg gaatatcatc ctgatttaga aaatttggat gaagatggat atactcaatt |
| 241 | acacttcgac tctcaaagca ataccaggat agctgttgtt tcagagaaag gatcgtgtgc |
| 301 | tgcatctcct ccttggcgcc tcattgctgt aattttggga atcctatgct tggtaatact |
| 361 | ggtgatagct gtggtcctgg gtaccatggc tatttggaga tccaattcag gaagcaacac |
| 421 | attggagaat ggctactttc tatcaagaaa taaagagaac cacagtcaac ccacacaatc |
| 481 | atctttagaa gacagtgtga ctcctaccaa agctgtcaaa accacagggg ttctttccag |
| 541 | cccttgtcct cctaattgga ttatatatga gaagagctgt tatctattca gcatgtcact |
| 601 | aaattcctgg gatggaagta aaagacaatg ctggcaactg ggctctaatc tcctaaagat |
| 661 | agacagctca aatgaattgg gatttatagt aaaacaagtg tcttcccaac ctgataattc |
| 721 | attttggata ggcctttctc ggccccagac tgaggtacca tggctctggg aggatggatc |
| 781 | aacattctct tctaacttat ttcagatcag aaccacagct acccaagaaa acccatctcc |
| 841 | aaattgtgta tggattcacg tgtcagtcat ttatgaccaa ctgtgtagtg tgccctcata |
| 901 | tagtatttgt gagaagaagt tttcaatgta agaggaaggg tggagaagga gagagaaata |
| 961 | tgtgaggtag taaggaggac agaaaacaga aaagaaaaga gtaacagctg aggtcaagat |
| 1021 | aaatgcagaa aatgtttaga gagcttggcc aactgtaatc ttaaccaaga aattgaaggg |
| 1081 | agaggctgtg atttctgtat ttgtcgacct acaggtaggc tagtattatt tttctagtta |
| 1141 | gtagatccct agacatggaa tcagggcagc caagattgag tttttatttt ttatttattt |
| 1201 | atttttttga gatagggtct cactttgtta cccaggctgg agtgcagtgg cacaatctcg |
| 1261 | actcactgca gctatctctc gcctcagccc ctcaagtagc tgggactaca ggtgcatgcc |
| 1321 | accatgccag gctaattttt ggtgtttttt gtagagactg ggttttgcca tgttgaccaa |
| 1381 | gctggtctct aactcctggg cttaagtgat ctgcccgcct tggcctccca aagtgctggg |
| 1441 | attacagatg tgagccacca cacctggccc caagcttgaa ttttcattct gccattgact |
| 1501 | tggcatttac cttgggtaag ccataagcga atcttaattt ctggctctat cagagttgtt |
| 1561 | tcatgctcaa caatgccatt gaagtgcacg gtgtgttgcc acgatttgac cctcaacttc |
| 1621 | tagcagtata tcagttatga actgagggtg aaatatattt ctgaatagct aaatgaagaa |
| 1681 | atgggaaaaa atattcacca cagtcagagc aattttatta ttttcatcag tatgatcata |
| 1741 | attatgatta tcatcttagt aaaaagcagg aactcctact ttttctttat caattaaata |
| 1801 | gctcagagag tacatctgcc atatctctaa tagaatcttt tttttttttt ttttttttga |
| 1861 | gacagagttt cgctcttgtt gcccaggctg gagtgcaacg gcacgatctc ggctcaccgc |
| 1921 | aacctccgcc ccctgggttc aagcaattct cctgcctcag cctcccaagt agctgggatt |
| 1981 | acagtcaggc accaccacac ccggctaatt ttgtattttt ttagtagaga cagggtttct |
| 2041 | ccatgtcggt cagggtagtc ccgaactcct gacctcaagt gatctgcctg cctcggcctc |
| 2101 | ccaagtgctg ggattacagg cgtgagccac tgcacccagc ctagaatctt gtataatatg |
| 2161 | taattgtagg gaaactgctc tcataggaaa gttttctgct ttttaaatac aaaaatacat |
| 2221 | aaaaatacat aaaatctgat gatgaatata aaaaagtaac caacctcatt ggaacaagta |
| 2281 | ttaacatttt ggaatatgtt ttattagttt tgtgatgtac tgttttacaa tttttaccat |
| 2341 | ttttttcagt aattactgta aaatggtatt attggaatga aactatattt cctcatgtgc |
| 2401 | tgatttgtct tatttttttc atactttccc actggtgcta tttttatttc caatggatat |
| 2461 | ttctgtatta ctagggaggc atttacagtc ctctaatgtt gattaatatg tgaaaagaaa |
| 2521 | ttgtaccaat tttactaaat tatgcagttt aaaatggatg attttatgtt atgtggattt |
| 2581 | catttcaata aaaaaaaact cttatcaaaa aaaaaaaaaa aa |

SEQ ID NO: 128 Human CLEC7A Isoform A Amino Acid Sequence (NP_922938.1)

| | |
|---|---|
| 1 | meyhpdlenl dedgytqlhf dsqsntriav vsekgscaas ppwrliavil gilclvilvi |
| 61 | avvlgtmaiw rsnsgsntle ngyflsrnke nhsqptqssl edsvtptkav kttgvlsspc |
| 121 | ppnwiiyeks cylfsmslns wdgskrqcwq lgsnllkids snelgfivkq vssqpdnsfw |
| 181 | iglsrpqtev pwlwedgstf ssnlfqirtt atqenpspnc vwihvsviyd qlcsvpsysi |
| 241 | cekkfsm |

SEQ ID NO: 129 Human CLEC7A Transcript Variant 2 cDNA Sequence
(NM_022570.4; CDS: 188-793)

| | |
|---|---|
| 1 | agcatagttt catttcctgc tcttgaatat ctggttgaac tacttaagct taatttgtta |
| 61 | aactccggta agtacctagc ccacatgatt tgactcagag attctctttt gtccacagac |
| 121 | agtcatctca ggagcagaaa gaaaagagct cccaaatgct atatctattc aggggctctc |
| 181 | aagaacaatg gaatatcatc ctgatttaga aaatttggat gaagatggat atactcaatt |
| 241 | acacttcgac tctcaaagca ataccaggat agctgttgtt tcagagaaag gatcgtgtgc |
| 301 | tgcatctcct ccttggcgcc tcattgctgt aattttggga atcctatgct tggtaatact |
| 361 | ggtgatagct gtggtcctgg gtaccatggg ggttatttcc agcccttgtc ctcctaattg |
| 421 | gattatatat gagaagagct gttatctatt cagcatgtca ctaaattcct gggatggaag |
| 481 | taaaagacaa tgctggcaac tgggctctaa tctcctaaag atagacagct caaatgaatt |
| 541 | gggatttata gtaaaacaag tgtcttccca acctgataat tcattttgga taggcctttc |
| 601 | tcggccccag actgaggtac catggctctg ggaggatgga tcaacattct cttctaactt |
| 661 | atttcagatc agaaccacag ctacccaaga aaacccatct ccaadttgtg tatggattca |
| 721 | cgtgtcagtc atttatgacc aactgtgtag tgtgccctca tatagtattt gtgagaagaa |
| 781 | gttttcaatg taagaggaag ggtggagaag gagagagaaa tatgtgaggt agtaaggagg |
| 841 | acagaaaaca gaacagaaaa gagtaacagc tgaggtcaag ataaatgcag aaaatgttta |
| 901 | gagagcttgg ccaactgtaa tcttaaccaa gaaattgaag ggagaggctg tgatttctgt |
| 961 | atttgtcgac ctacaggtag gctagtatta tttttctagt tagtagatcc ctagacatgg |
| 1021 | aatcagggca gccaagcttg agtttttatt ttttatttat ttattttttt gagatagggt |
| 1081 | ctcactttgt tacccaggct ggagtgcagt ggcacaatct cgactcactg cagctatctc |
| 1141 | tcgcctcagc ccctcaagta gctgggacta caggtgcatg ccaccatgcc aggctaattt |
| 1201 | ttggtgtttt ttgtagagac tgggttttgc catgttgacc aagctggtct ctaactcctg |
| 1261 | ggcttaagtg atctgcccgc cttggcctcc caaagtgctg ggattacaga tgtgagccac |
| 1321 | cacacctggc cccaagcttg aattttcatt ctgccattga cttggcattt accttgggta |
| 1381 | agccataagc gaatcttaat ttctggctct atcagagttg tttcatgctc aacaatgcca |
| 1441 | ttgaagtgca cggtgtgttg ccacgatttg accctcaact tctagcagta tatcagttat |
| 1501 | gaactgaggg tgaaatatat ttctgaatag ctaaatgaag aaatgggaaa aaatcttcac |
| 1561 | cacagtcaga gcaattttat tattttcatc agtatgatca taattatgat tatcatctta |
| 1621 | gtaaaaagca ggaactccta ctttttcttt atcaattaaa tagctcagag agtacatctg |
| 1681 | ccatatctct aatagaatct tttttttttt tttttttttt gagacagagt ttcgctcttg |
| 1741 | ttgcccaggc tggagtgcaa cggcacgatc tcggctcacc gcaacctccg ccccctgggt |
| 1801 | tcaagcaatt ctcctgcctc agcctcccaa gtagctggga ttacagtcag gcaccaccac |
| 1861 | acccggctaa ttttgtattt ttttagtaga gacagggttt ctccatgtcg gtcagggtag |
| 1921 | tcccgaactc ctgacctcaa gtgatctgcc tgcctcggcc tcccaagtgc tgggattaca |
| 1981 | ggcgtgagcc actgcaccca gcctagaatc ttgtataata tgtaattgta gggaaactgc |
| 2041 | tctcatagga aagttttctg ctttttaaat acaaaaatac ataaaaatac ataaaatctg |
| 2101 | atgatgaata taaaaaagta accaacctca ttggaacaag tattaacatt ttggaatatg |
| 2161 | ttttattagt tttgtgatgt actgttttac aatttttacc atttttttca gtaattactg |
| 2221 | taaaatggta ttattggaat gaaactatat ttcctcatgt gctgatttgt cttatttttt |
| 2281 | tcatactttc ccactggtgc tatttttatt tccaatggat atttctgtat tactagggag |
| 2341 | gcatttacag tcctctaatg ttgattaata tgtgaaaaga aattgtacca attttactaa |
| 2401 | attatgcagt ttaaaatgga tgattttatg ttatgtggat ttcatttcaa taaaaaaaaa |
| 2461 | ctcttatcaa aaaaaaaaaa aaaa |

SEQ ID NO: 130 Human CLEC7A Isoform B Amino Acid Sequence (NP_072092.2)

| | |
|---|---|
| 1 | meyhpdlenl dedgytqlhf dsqsntriav vsekgscaas ppwrliavil gilcivilvi |
| 61 | avvlgtmgvl sspcppnwii yekscylfsm slnswdgskr qcwqlgsnll kidssnelgf |
| 121 | ivkqvssqpd nsfwiglsrp qtevpwlwed gstfssnlfq irttatqenp spncywihvs |
| 181 | viydqlcsvp sysicekkfs m |

SEQ ID NO: 13 Human CLEC7A Transcript Variant 3 cDNA Sequence
(NM_197948.2; CDS: 188-757)

| | |
|---|---|
| 1 | agcatagttt catttcctgc tcttgaatat ctggttgaac tacttaagct taatttgtta |
| 61 | aactccggta agtacctagc ccacatgatt tgactcagag attctctttt gtccacagac |
| 121 | agtcatctca ggagcagaaa gaaaagagct cccaaatgct atatctattc aggggctctc |
| 181 | aagaacaatg gaatatcatc ctgatttaga aaatttggat gaagatggat atactcaatt |
| 241 | acacttcgac tctcaaagca ataccaggat agctgttgtt tcagagaaag gatcgtgtgc |
| 301 | tgcatctcct ccttggcgcc tcattgctgt aattttggga atcctatgct tggtaatact |
| 361 | ggtgatagct gtggtcctgg gtaccatggc tatttggaga tccaattcag gaagcaacac |
| 421 | attggagaat ggctactttc tatcaagaaa taaagagaac cacagtcaac ccacacaatc |
| 481 | atctttagaa gacagtgtga ctcctaccaa agctgtcaaa accacagggg ttctttccag |
| 541 | cccttgtcct cctaattgga ttatatatga gaagagctgt tatctattca gcatgtcact |
| 601 | aaattcctgg gatggaagta aaagacaatg ctggcaactg ggctctaatc tcctaaagat |
| 661 | agacagctca aatgaattga tttcagatca gaaccacagc tacccaagaa aacccatctc |
| 721 | caaattgtgt atggattcac gtgtcagtca tttatgacca actgtgtagt gtgccctcat |
| 781 | atagtatttg tgagaagaag ttttcaatgt aagaggaagg gtggagaagg agagagaaat |
| 841 | atgtgaggta gtaaggagga cagaaaacag aacagaaaag agtaacagct gaggtcaaga |
| 901 | taaatgcaga aaatgtttag agagcttggc caactgtaat cttaaccaag aaattgaagg |
| 961 | gagaggctgt gatttctgta tttgtcgacc tacaggtagg ctagtattat ttttctagtt |
| 1021 | agtagatccc tagacatgga atcagggcag ccaagcttga gtttttattt tttatttatt |
| 1081 | tatttttttg agatagggtc tcactttgtt acccaggctg gagtgcagtg gcacaatctc |
| 1141 | gactcactgc agctatctct cgcctcagcc cctcaagtag ctgggactac aggtgcatgc |
| 1201 | caccatgcca ggctaatttt tggtgttttt tgtagagact gggttttgcc atgttgacca |
| 1261 | agctggtctc taactcatgg gcttaagtga tctgcccgcc ttggcctccc aaagtgctgg |
| 1321 | gattacagat gtgagccacc acacgtggcc ccaagcttga attttcattc tgccattgac |
| 1381 | ttggcattta ccttgggtaa gccataagcg aatcttaatt tctggctcta tcagagttgt |
| 1441 | ttcatgctca acaatgccat tgaagtgcac ggtgtgttgc cacgatttga ccatcaactt |
| 1501 | ctagcagtat atcagttatg aactgagggt gaaatatatt tctgaatagc taaatgaaga |
| 1561 | aatgggaaaa aatcttcacc acagtcagag caattttatt attttcatca gtatgatcat |
| 1621 | aattatgatt atcatcttag taaaaagcag gaactcctac tttttcttta tcaattaaat |
| 1681 | agctcagaga gtacatctgc catatctcta atagaatctt tttttttttt tttttttttg |
| 1741 | agacagagtt tcgctcttgt tgcccaggct ggagtgcaac ggcacgatct cggctcaccg |
| 1801 | caacctccgc cccctgggtt caagcaattc tcctgcctca gcctcccaag tagctgggat |
| 1861 | tacagtcagg caccaccaca cccggctaat tttgtatttt tttagtagag acagggtttc |
| 1921 | tccatgtcgg tcagggtagt cccgaactcc tgacctcaag tgatctgcct gcctcggcct |
| 1981 | cccaagtgct gggattacag gagtgagcca ctgcacccag cctagaatct tgtataatat |
| 2041 | gtaattgtag ggaaactgct ctcataggaa agttttctgc tttttaaata caaaaataca |
| 2101 | taaaaataca taaaatctga tgatgaatat aaaaaagtaa ccaacctcat tggaacaagt |
| 2161 | attaacattt tggaatatgt tttattagtt ttgtgatgta ctgttttaca atttttacca |
| 2221 | tttttttcag taattactgt aaaatggtat tattggaatg aaactatatt tcctcatgtg |
| 2281 | ctgatttgtc ttattttttt catactttcc cactggtgct atttttattt ccaatggata |
| 2341 | tttctgtatt actagggagg catttacagt cctctaatgt tgattaatat gtgaaaagaa |
| 2401 | attgtaccaa ttttactaaa ttatgcagtt taaaatggat gattttatgt tatgtggatt |
| 2461 | tcatttcaat aaaaaaaaac tcttatcaaa aaaaaaaaaa aaa |

SEQ ID NO: 132 Human CLEC7A Isoform C Amino Acid Sequence (NP_922939.1)

| | |
|---|---|
| 1 | meyhpdlenl dedgytqlhf dsqsntriav vsekgscaas ppwrliavil gilclvilvi |
| 61 | avvlgtmaiw rsnsgsntle ngyflsrnke nhsqptqssl edsvtptkav kttgvlsspc |
| 121 | ppnwiiyeks cylfsmslns wdgskrqcwq lgsnllkids snelisdqnh syprkpiskl |
| 181 | cmdsrvshl |

SEQ ID NO: 133 Human CLEC7A Transcript Variant 4 cDNA Sequence
(NM_197949.2)

| | |
|---|---|
| 1 | agcatagttt catttcctgc tcttgaatat ctggttgaac tacttaagct taatttgtta |
| 61 | aactccggta agtacctagc ccacatgatt tgactcagag attctctttt gtccacagac |
| 121 | agtcatctca ggagcagaaa gaaaagagct cccaaatgct atatctattc aggggctctc |
| 181 | aagaacaatg gaatatcatc ctgatttaga aaatttggat gaagatggat atactcaatt |
| 241 | acacttcgac tctcaaagca ataccaggat agctgttgtt tcagagaaag gatcgtgtgc |
| 301 | tgcatctcct ccttggcgcc tcattgctgt aattttggga atcctatgct tggtaatact |
| 361 | ggtgatagct gtggtcctgg gtaccatggg ggttctttcc agcccttgtc ctcctaattg |
| 421 | gattatatat gagaagagct gttatctatt cagcatgtca ctaaattcct gggatggaag |
| 481 | taaaagacaa tgctggcaac tgggctctaa tctcctaaag atagacagct caaatgaatt |
| 541 | gatttcagat cagaaccaca gctacccaag aaaacccatc tccaaattgt gtatggattc |
| 601 | acgtgtcagt catttatgac caactgtgta gtgtgccctc atatagtatt tgtgagaaga |
| 661 | agttttcact gtaagaggaa gggtggagaa ggagagagaa atatgtgagg tagtaaggag |
| 721 | gacagaaaac agaacagaaa agagtaacag ctgaggtcaa gataaatgca gaaaatgttt |
| 781 | agagagcttg gccaactgta atcttaacca agaaattgaa gggagaggct gtgatttctg |
| 841 | tatttgtcga cctacaggta ggctagtatt atttttctag ttagtagatc cctagacatg |
| 901 | gaatcagggc agccaagctt gagtttttat tttttattta tttatttttt tgagataggg |
| 961 | tctccctttg ttacccaggc tggagtgcag tggcacaatc tcgactcact gcagctatct |
| 1021 | ctcgcctcag cccctcaagt agctgggact acaggtgcat gccaccatgc caggctaatt |
| 1081 | tttggtgttt tttgtagaga ctgggttttg ccatgttgac caagctggtc tctaactcct |
| 1141 | gggcttaagt gatctgcccg ccttggcctc ccaaagtgct gggattacag atgtgagcca |
| 1201 | ccacacctgg ccccaagctt gacttttcat tctgccattg acttggcatt taccttgggt |
| 1261 | aagccataag cgaatcttaa tttctggctc tatcagagtt gtttcatgct caacaatgcc |
| 1321 | attgaagtgc acggtgtgtt gccacgattt gaccctcaac ttctagcagt atatcagtta |
| 1381 | tgaactgagg gtgaaatata tttctgaata gctaaatgaa gaaatgggaa aaaatcttca |
| 1441 | ccacagtcag agcaatttta ttattttcat cagtatgatc ataattatga ttatcatctt |
| 1501 | agtaaaaagc aggaactcct actttttctt tatcaattaa atagctcaga gagtacctct |
| 1561 | gccatatctc taatagaatc tttttttttt tttttttttt tgagacagag tttcgctctt |
| 1621 | gttgcccagg ctggagtgca acggcccgat ctcggctcac cgcaacctcc gccccctggg |
| 1681 | ttcaagcaat tctcctgcct cagcctccca agtagctggg attacagtca ggcaccacca |
| 1741 | cacccggcta attttgtatt tttttagtag agacagggtt tctccatgtc ggtcagggta |
| 1801 | gtcccgaact cctgacctca agtgatctgc ctgcctcggc ctcccaagtg ctgggattac |
| 1861 | aggcgtgagc cactgcaccc agcctagaat cttgtataat atgtaattgt agggaaactg |
| 1921 | ctctcatagg aaagttttct gctttttaaa tacaaaaata cataaaaata cataaaatct |
| 1981 | gatgatgaat ataaaaaagt aaccaacctc attggaacaa gtattaacat tttggaatat |
| 2041 | gttttattag ttttgtgatg tactgtttta caatttttac catttttttc agtaattact |
| 2101 | gtaaaatggt attattggaa tgaaactata tttcctcatg tgctgatttg tcttattttt |
| 2161 | ttcatacttt cccactggtg ctatttttat ttccaatgga tatttctgta ttactaggga |
| 2221 | ggcatttaca gtcctctaat gttgattaat atgtgaaaag aaattgtacc aattttacta |
| 2281 | aattatgcag tttaaaatgg atgattttat gttatgtgga tttcatttca ataaaaaaaa |
| 2341 | actcttatca aaaaaaaaaa aaaaa |

SEQ ID NO: 134 Human CLEC7A Isoform D Amino Acid Sequence (NP_922940.1)

| | |
|---|---|
| 1 | meyhpdlenl dedgytqlhf dsqsntriav vsekgscaas ppwrliavil gilclvilvi |
| 61 | avvlgtmgvl sspcppnwii yekscylfsm slnswdgskr qcwqlgsnll kidssnelis |
| 121 | dcmhsyprkp isklcmdsrv shl |

SEQ ID NO: 135 Human CLEC7A Transcript Variant 5 cDNA Sequence
(NM_197950.2; CDS: 188-694)

| | |
|---|---|
| 1 | agcatagttt catttcctgc tcttgaatat ctggttgaac tacttaagct taatttgtta |
| 61 | aactccggta agtacctagc ccacatgatt tgactcagag attctctttt gtccacagac |
| 121 | agtcatctca ggagcagaaa gaaaagagct cccaaatgct atatctattc aggggctctc |
| 181 | aagaacaatg gaatatcatc ctgatttaga aaatttggat gaagatggat atactcaatt |
| 241 | acacttcgac tctcaaagca ataccaggat agctgttgtt tcagagaaag gggttctttc |
| 301 | cagcccttgt cctcctaatt ggattatata tgagaagagc tgttatctat tcagcatgtc |
| 361 | actaaattcc tgggatggaa gtaaaagaca atgctggcaa ctgggctcta atctcctaaa |
| 421 | gatagacagc tcaaatgaat tgggatttat agtaaaacaa gtgtcttccc aacctgataa |
| 481 | ttcattttgg ataggccttt ctcggcccca gactgaggta ccatggctct gggaggatgg |
| 541 | atcaacattc tcttctaact tatttcagat cagaaccaca gctacccaag aaaacccatc |
| 601 | tccaaattgt gtatggattc acgtgtcagt catttatgac caactgtgta gtgtgccctc |
| 661 | atatagtatt tgtgagaaga agttttcaat gtaagaggaa gggtggagaa ggagagagaa |
| 721 | atatgtgagg tagtaaggag gacagaaaac agaacagaaa agagtaacag ctgaggtcaa |
| 781 | gataaatgca gaaaatgttt agagagcttg gccaactgta atcttaacca agaaattgaa |
| 841 | gggagaggct gtgatttctg tatttgtcga cctacaggta ggctagtatt atttttctag |
| 901 | ttagtagatc cctagacatg gaatcagggc agccaagctt gagtttttat tttttattta |
| 961 | tttatttttt tgagataggg tctcactttg ttacccaggc tggagtgcag tggcacaatc |
| 1021 | tcgactcact gcagctatct ctcgcctcag cccctcaagt agctgggact acaggtgcat |
| 1081 | gccaccatgc caggctaatt tttggtgttt tttgtagaga ctgggttttg ccatgttgac |
| 1141 | caagctggtc tctaactcct gggcttaagt gatctgcccg ccttggcctc ccaaagtgct |
| 1201 | gggattacag atgtgagcca ccacacctgg ccccaagctt gaattttcat tctgccattg |
| 1261 | acttggcatt taccttgggt aagccataag cgaatcttaa tttctggctc tatcagagtt |
| 1321 | gtttcatgct caacaatgcc attgaagtgc acggtgtgtt gccacgattt gaccctcaac |
| 1381 | ttctagcagt atatcagtta tgaactgagg gtgaaatata tttctgaata gctaaatgaa |
| 1441 | gaaatgggaa aaaatcttca ccacagtcag agcaatttta ttattttcat cagtatgatc |
| 1501 | ataattatga ttatcatctt agtaaaaagc aggaactcct actttttctt tatcaattaa |
| 1561 | atagctcaga gagtacatct gccatatctc taatagaatc tttttttttt tttttttttt |
| 1621 | tgagacagag tttcgctctt gttgcccagg ctggagtgca acggcacgat ctcggctcac |
| 1681 | cgcaacctcc gccccctggg ttcaagcaat tctcctgcct cagcctccca agtagctggg |
| 1741 | attacagtca ggcaccacca cacccggcta attttgtatt tttttagtag agacagggtt |
| 1801 | tctccatgtc ggtcagggta gtcccgaact cctgacctca agtgatctgc ctgcctcggc |
| 1861 | ctcccaagtg ctgggattac aggcgtgagc cactgcaccc agcctagaat cttgtataat |
| 1921 | atgtaattgt agggaaactg ctctcatagg aaagttttct gctttttaaa tacaaaaata |
| 1981 | cataaaaata cataaaatct gatgatgaat ataaaaaagt aaccaacctc attggaacaa |
| 2041 | gtattaacat tttggaatat gttttattag ttttgtgatg tactgtttta caatttttac |
| 2101 | catttttttc agtaattact gtaaaatggt attattggaa tgaaactata tttcctcatg |
| 2161 | tgctgatttg tcttattttt ttcatacttt cccactggtg ctatttttat ttccaatgga |
| 2221 | tatttctgta ttactaggga ggcatttaca gtcctctaat gftgattaat atgtgaaaag |
| 2281 | aaattgtacc aattttacta aattatgcag tttaaaatgg atgattttat gttatgtgga |
| 2341 | tttcatttca ataaaaaaaa actcttatca aaaaaaaaaa aaaaa |

SEQ ID NO: 136 Human CLEC7A Isoform E Amino Acid Sequence (NP_922941.1)

| | |
|---|---|
| 1 | meyhpdlenl dedgytqlhf dsqsntriav vsekgvlssp cppnwiiyek scylfsmsln |
| 61 | swdgskrqcw qlgsnllkid ssnelgfivk qvssqpdnsf wiglsrpqte vpwlwedgst |
| 121 | fssnlfqirt tatqenpspn cvwihvsviy dqlcsvpsys icekkfsm |

SEQ ID NO: 137 Human CLEC7A Transcript Variant 6 cDNA Sequence
(NM_197954.2; CDS: 188-421)

| | |
|---|---|
| 1 | agcatagttt catttcctgc tcttgaatat ctggttgaac tacttaagct taatttgtta |
| 61 | aactccggta agtacctagc ccacatgatt tgactcagag attctctttt gtccacagac |
| 121 | agtcatctca ggagcagaaa gaaaagagct cccaaatgct atatctattc aggggctctc |
| 181 | aagaacaatg gaatatcatc ctgatttaga aaatttggat gaagatggat atactcaatt |
| 241 | acacttcgac tctcaaagca ataccaggat agctgttgtt tcagagaaag gatcgtgtgc |
| 301 | tgcatctcct ccttggcgcc tcattgctgt aattttggga atcctatgct tggtaatact |
| 361 | ggtgatagct gtggtcctgg gtaccatggc tggtttcaaa gctgtggaat tcaaaggata |
| 421 | aattaatgaa gaaaacaagc ggagctgaag aagaaagtac aatatggtgc tgtcttccta |
| 481 | atgaaataaa ttcactaaat ggacattaaa aaaaaaaa |

SEQ ID NO: 138 Human CLEC7A Isoform F Amino Acid Sequence (NP_922945.1)

| | |
|---|---|
| 1 | meyhpdlenl dedgytqlhf dsqsntriav vsekgscaas ppwrliavil gilclvilvi |
| 61 | avvlgtmagf kavefkg |

SEQ ID NO: 139 Mouse CLEC7A Transcript Variant 2 cDNA Sequence
(NM_001309637.1; CDS: 95-694)

| | |
|---|---|
| 1 | tgtttcaggg tttgggttag tgagcctcat cctggcagtt attttatagt aaagaacatt |
| 61 | caagtgctct gcctacctag ggccctgtga agcaatgaaa tatcactctc atatagagaa |
| 121 | tctggatgaa gatggatata ctcaattaga cttcagcact caagacatcc ataaaaggcc |
| 181 | caggggatca gagaaaggaa gccaggctcc atcttcacct tggaggccca ttgcagtggg |
| 241 | tttaggaatc ctgtgctttg tggtagtagt ggttgctgca gtgctgggtg ccctaggagg |
| 301 | tttttctcag ccttgccttc ctaattggat catgcatggg aagagctgtt acctatttag |
| 361 | cttctcagga aattcctggt atggaagtaa gagacactgc tcccagctag gtgctcatct |
| 421 | actgaagata gacaactcaa aagaatttga gttcattgaa agccaaacat cgtctcaccg |
| 481 | tattaatgca ttttggatag gcctttcccg caatcagagt gaagggccat ggttctggga |
| 541 | ggatggatca gcattcttcc ccaactcgtt tcaagtcaga aatacagctc cccaggaaag |
| 601 | cttactgcac aattgtgtat ggattcatgg atcagaggtc tacaaccaaa tctgcaatac |
| 661 | ttattcatac agtatctgtg agaaggaact gtaaatgtat gtgagaatat aaagatggtg |
| 721 | tgtgtgtgtg tgtgtgtgtg tgtgtacatg cacacacacc accaccacca ccactaccaa |
| 781 | caacagaaca gaacagaaca gaacagaaca gattaatatt aaaaaacaga aaaaatgctg |
| 841 | ggatgctaag agactttaac ctcatttgag aacttggatg aagaagctga gacttttgta |
| 901 | cttgtcatct tcacaaagat ggtggcacta tcttccagtt aggaagtcac tagacatgga |
| 961 | gtgagggcag ctcaacaata cagagaatat gtgaacctga ggtaccctga ctcaaatttc |
| 1021 | acaaccacaa tgaaacccct acactatcag gaaacactgt agaggagtga gactgaagac |
| 1081 | tttaaaagcc agagaatcag cctacttact gtggtgtttt ctagacagga cagggaaagt |
| 1141 | atatctagga aataaaaaca atacaattca gcaaacaaaa tctgcataat gacaacctca |
| 1201 | gttggtatgg tatgttatgg tatggtatgg gtgtagaagt ttcacaaggc cctatgaaga |
| 1261 | actacagaca gttaaatagg gggaaagctt tttctaggat caagcctact gaaccccaag |
| 1321 | aagtcagcac tgaacatatg tacagatcag tatcattaaa tgaactagta agacatatac |
| 1381 | atatatgtta atcaaatatt ggtaccagag tacacactgt gtttgcatga ttttctcagt |
| 1441 | atctacagta caccagacac agggagaagg caaaatgaac ttctaaattg agaagtgaaa |
| 1501 | aaaatgaggg aagagaatct tcaccacaaa tagggattct attttcaccc acatgatcat |
| 1561 | tattaagatg gccatcaccc aaacgtcgtg acccaagcta cttcctcaac tagataactc |
| 1621 | aaagagtctg cccacctttt ctgatagcaa atctggtatc tagatttcac tgtttcctta |
| 1681 | tgctgtctgg ccagcagtat gacaaaggtg ctgccctttc aggaagcagt ctccttaaat |
| 1741 | gctgtagttg gaaagataaa tcatatctga tagtgaatat ttaaaaagcg cccagtcagg |
| 1801 | ataagtgttt tggaacacag aacatatttc atctttttat gatacactat cttgcaatta |
| 1861 | acaaccaatt cttaagtcat ttctttacaa acatatgact ggaatatgac tgtttcctag |
| 1921 | tgtgatctgt cttgttaact tctaagattg tccattaata ccacccttat ttccagtgtg |
| 1981 | gacttccaaa ttgctgggga tctgtttata gctttctcag actaatcaat atgtgggcag |
| 2041 | aaattgtgct gagtccactg aattgttctc ttgaaaatga ttgggtttat gtcactttca |
| 2101 | tctcaattga aaaactgctt attaaagtat ctttggcctc tgaa |

SEQ ID NO: 140 Mouse CLEC7A Isoform 2 Amino Acid Sequence
(NP_001296566.1)

| | |
|---|---|
| 1 | mkyhshienl dedgytqldf stqdihkrpr gsekgsqaps spwrpiavgl gilcfvvvvv |
| 61 | aavlgalggf sqpclpnwim hgkscylfsf sgnswygskr hcsqlgahll kidnskefef |
| 121 | iesqtsshri nafwiglsrn qsegpwfwed gsaffpnsfq vrntapqesl lhncvwihgs |
| 181 | evynqicnts sysicekel |

SEQ ID NO: 141 Mouse CLEC7A Transcript Variant 1 cDNA Sequence
(NM_020008.3; CDS: 95-829)

| | |
|---|---|
| 1 | tgtttcaggg tttgggttag tgagcctcat cctggcagtt attttatagt aaagaacatt |
| 61 | caagtgctct gcctacctag ggccctgtga agcaatgaaa tatcactctc atatagagaa |
| 121 | tctggatgaa gatggatata ctcaattaga cttcagcact caagacatcc ataaaaggcc |
| 181 | caggggatca gagaaaggaa gccaggctcc atcttcacct tggaggccca ttgcagtggg |
| 241 | tttaggaatc ctgtgctttg tggtagtagt ggttgctgca gtgctgggtg ccctagcatt |
| 301 | ttggcgacac aattcaggga gaaatccaga ggagaaagac aacttcctat caagaaataa |
| 361 | agagaaccac aagcccacag aatcatcttt agatgagaag gtggctccct ccaaggcatc |
| 421 | ccaaactaca ggaggttttt ctcagccttg ccttcctaat tggatcatgc atgggaagag |
| 481 | ctgttaccta tttagcttct caggaaattc ctggtatgga agtaagagac actgctccca |
| 541 | gctaggtgct catctactga agatagacaa ctcaaaagaa tttgagttca ttgaaagcca |
| 601 | aacatcgtct caccgtatta atgcattttg gataggcctt tcccgcaatc agagtgaagg |
| 661 | gccatggttc tgggaggatg gatcagcatt cttccccaac tcgtttcaag tcagaaatac |
| 721 | agctccccag gaaagcttac tgcacaattg tgtatggatt catggatcag aggtctacaa |
| 781 | ccaaatctgc aatacttatt catacagtat ctgtgagaag gaactgtaaa tgtatgtgag |
| 841 | aatataaaga tggtgtgtgt gtgtgtgtgt gtgtgtgtgt acatgcacac acaccaccac |
| 901 | caccaccact accaacaaca gaacagaaca gaacagaaca gaacagatta atattaaaaa |
| 961 | acagaaaaaa tgctgggatg ctaagagact ttaacctcat ttgagaactt ggatgaagaa |
| 1021 | gctgagactt ttgtacttgt catcttcaca aagatggtgg cactatcttc cagttaggaa |
| 1081 | gtcactagac atggagtgag ggcagctcaa caatacagag aatatgtgaa cctgaggtac |
| 1141 | cctgactcaa atttcacaac cacaatgaaa cccctacact atcaggaaac aatgtagagg |
| 1201 | agtgagactg aagactttaa aagccagaga atcagcctac ttactgtggt gttttctaga |
| 1261 | caggacaggg aaagtatatc taggaaataa aaacaataca attcagcaaa caaaatctgc |
| 1321 | ataatgacaa cctcagttgg tatggtatgt tatggtatgg tatgggtgta gaagtttcac |
| 1381 | aaggccctat gaagaactac agacagttaa atagggggaa agctttttct aggatcaagc |
| 1441 | ctactgaacc ccaagaagtc agcactgaac atatgtacag atcagtatca ttaaatgaac |
| 1501 | tagtaagaca tatacatata tgttaatcaa atattggtac cagagtacac actgtgtttg |
| 1561 | catgattttc tcagtatcta cagtacacca gacacaggga gaaggcaaaa tgaacttcta |
| 1621 | aattgagaag tgaaaaaaat gagggaagag aatcttcacc acaaataggg attctatttt |
| 1681 | cacccacatg atcattatta agatggccat cacccaaacg tcgtgaccca agctacttcc |
| 1741 | tcaactagat aactcaaaga gtctgcccac cttttctgat agcaaatctg gtatctagat |
| 1801 | ttcactgttt ccttatgctg tctggccaga agtatgacaa aggtgctgcc ctttcaggaa |
| 1861 | gcagtctcct taaatgctgt agttggaaag ataaatcata tctgatagtg aatatttaaa |
| 1921 | aagcgcccag tcaggataag tgttttggaa cacagaacat atttcatctt tttatgatac |
| 1981 | actatcttgc aattaacaac caattcttaa gtcatttctt tacaaacata tgactggaat |
| 2041 | atgactgttt cctagtgtga tctgtcttgt taacttctaa gattgtccat taataccacc |
| 2101 | cttatttcca gtgtggactt ccaaattgct ggggatctgt ttatagcttt ctcagactaa |
| 2161 | tcaatatgtg ggcagaaatt gtgctgagtc cactgaattg ttctcttgaa aatgattggg |
| 2221 | tttatgtcac tttcatctca attgaaaaac tgcttattaa agtatctttg gcctctgaa |

SEQ ID NO: 142 Mouse CLEC7A Isoform 1 Amino Acid Sequence (NP_064392.2)

| | |
|---|---|
| 1 | mkyhshienl dedgytqldf stqdihkrpr gsekgsqaps spwrpiavgl gilcfvvvvv |
| 61 | aavlgalafw rhnsgrnpee kdnflsrnke nhkptessld ekvapskasq ttggfsqpcl |
| 121 | pnwimhgksc ylfsfsgnsw ygskrhcsql gahllkidns kefefiesqt sshrinafwi |
| 181 | glsrnqsegp wfwedgsaff pnsfqvrnta pqesllhncv wihgsevynq icntssysic |
| 241 | ekei |

SEQ ID NO: 143 Human TBXAS1 Transcript Variant 1 cDNA Sequence
(NM_001061.4; CDS: 236-1840)

| | |
|---|---|
| 1 | aaggaataaa gttgctgatt cattccttta cactgaaacc ctttgttgtg ccctcctctt |
| 61 | ccttcctctt tatagggaga cactctgaga aagagcacat tgtgggggcc cactccatgt |
| 121 | gatgtttgct tggttgcctg ttcccttttc tacctgcaga gcacggttcc cataagggcg |
| 181 | gcgagatcag cctcctgtct catctggaag accaccactc tggggtctca gaggaatgat |
| 241 | ggaagccttg gggtttctaa aattggaagt gaatggcccc atggtgacgg tggccctgtc |
| 301 | agtggctctc ttggccctcc tgaaatggta ctccacatca gcattctcaa gactggagaa |
| 361 | gttaggcctc agacatccca agccttctcc tttcattgga aacttgacat ttttccgcca |
| 421 | gggtttttgg gaaagccaaa tggagctcag aaagctgtat ggacctctgt gtgggtacta |
| 481 | tcttggtcgt cggatgttta ttgttatttc tgagccagac atgatcaagc aggtgttggt |
| 541 | tgagaacttc agtaacttta ccaacagaat ggcgtcgggt ttggagttca agtcggtagc |
| 601 | cgacagcgtt ctgtttttac gtgacacaag atgggaagag gtcagaggtg ccctgatgtc |
| 661 | tgctttcagt cctgaaaagc tgaacgagat ggttcccctc atcagccaag cctgcgacct |
| 721 | tctcctggct catttaaaac gctatgcgga atctggggac gcatttgaca tccagaggtg |
| 781 | ctactgcaat tacaccacag atgtggttgc cagcgtcgcc tttggcaccc cggtggactc |
| 841 | ctggcaggcc cctgaggatc cctttgtgaa acactgcaag cgtttcttcg aattctgcat |
| 901 | ccccagacct atcctggttt tactcttatc atttccatcc ataatggtcc cactggcccg |
| 961 | gattttgccc aataagaacc gagacgaact gaatggcttt tttaacaaac tcattaggaa |
| 1021 | tgtgattgcc ttgcgggacc agcaagctgc cccagagagg cggagagact tcctccaaat |
| 1081 | ggtcctggat gcccgacatt ctgcaagtcc catgggcgtg caagactttg acatcgtcag |
| 1141 | agacgttttc tcctctactg ggtgcaagcc gaacccttcc cggcaacacc agcccagccc |
| 1201 | tatggccagg cctttgactg tggatgagat tgtgggccag gccttcatct tcctcatcgc |
| 1261 | tggctatgaa atcatcacca acacactttc ttttgccacc tacctactgg ccaccaaccc |
| 1321 | tgactgccaa gagaagcttc tgagagaggt agacgttttt aaggagaaac acatggcccc |
| 1381 | tgagttctgc agcctcgagg aaggcctgcc ctatctggac atggtgattg cagagacgct |
| 1441 | gaggatgtac ccgccagctt tcagattcac acgggaggca gctcaggact gcgaggtgct |
| 1501 | ggggcagcgc atccccgccg gcgctgtgct agagatggcc gtgggtgccc tgcaccatga |
| 1561 | ccctgagcac tggccaagcc cggagacctt caaccctgaa aggttcacgg ctgaggcccg |
| 1621 | gcagcagcac cggcccttca cgtacctgcc cttcggggcc ggcccacgga gctgcctcgg |
| 1681 | ggtgcgtcta gggctgcttg aggtcaagtt gacactgctc cacgtgctgc accagttccg |
| 1741 | gttccaagcc tgccctgaga cccaggtacc gctgcagcta gaatccaaat ctgccctagg |
| 1801 | tccaaaaaat ggtgtctata tcaagatcgt atcccgctga cacagaaggc tgccgggtgg |
| 1861 | ggggagggca cccccaaatt caaagaaaac cctaagtgtg gatgttcaga attttggaaa |
| 1921 | aatgtcactg aagtgattga aagagtgcct ggcatgcaag gataagaggt tctttacata |
| 1981 | acatttccta aatgcttaat aaacgtttgt tgcacttggt tttgacattg ccaatggggt |
| 2041 | ttgaaccagt gctctctcta aatgaaaaaa aaaaaaaaaa aa |

SEQ ID NO: 144 Human TBXAS1 Isoform 1 Amino Acid Sequence (NP_001052.2
and NP_001124438.1)

| | |
|---|---|
| 1 | mmealgflkl evngpmvtva lsvallallk wystsafsrl eklglrhpkp spfignltff |
| 61 | rqgfwesqme lrklygplcg yylgrrmfiv isepdmikqv lvenfsnftn rmasglefks |
| 121 | vadsvlflrd krweevrgal msafspekln emvplisqac dlllahlkrv aesgdafdiq |
| 181 | rcycnyttdv vasvafgtpv dswqapedpf vkhckrffef ciprpilvll lsfpsimvpl |
| 241 | arilpnknrd elngffnkli rnvialrdqq aaeerrrdfl qmvldarhsa spmgvqdfdi |
| 301 | vrdvfsstgc kpnpsrqhqp spmarpltvd eivgqafifl iagyeiitnt lsfatyllat |
| 361 | npdcqekllr evdvfkekhm apefcsleeg lpyldmviae tlrmyppafr ftreaaqdce |
| 421 | vlgqripaga vlemavgalh hdpehwpspe tfnperftae arqqhrpfty lpfgagprsc |
| 481 | lgvrlgllev kltllhvlhk flfqacpetq vplqlesksa lgpknqvyik ivsr |

SEQ ID NO: 145 Human TBXAS1 Transcript Variant 2 cDNA Sequence
(NM_030984.3; CDS: 236-1618)

| | |
|---|---|
| 1 | aaggaataaa gttgctgatt cattccttta cactgaaacc ctttgttgtg ccctcctctt |
| 61 | ccttcctctt tatagggaga cactctgaga aagagcacat tgtgggggcc cactccatgt |
| 121 | gatgtttgct tggttgcctg ttcccttttc tacctgcaga gcacggttcc cataagggcg |
| 181 | gcgagatcag cctcctgtct catctggaag accaccactc tggggtctca gaggaatgat |
| 241 | ggaaagcctt gggtttctaa aattggaagt gaatggcccc atggtgacgg tggccctgtc |
| 301 | agtggctctc ttggccctcc tgaaatggta ctccacatca gcattctcaa gactggagaa |
| 361 | gttaggcctc agacatccca agccttctcc tttcattgga aacttgacat ttttccgcca |
| 421 | gggtttttgg gaaagccaaa tggagctcag aaagctgtat ggacctctgt gtgggtacta |
| 481 | tcttggtcgt cggatgttta ttgttatttc tgagccagac atgatcaagc aggtgttggt |
| 541 | tgagaccttc agtaacttta ccaacagaat ggcgtcgggt ttggagttca agtcggtagc |
| 601 | cgacagcgtt ctgtttttac gtgacaaaag atgggaagag gtcagaggtg ccctgatgtc |
| 661 | tgctttcagt cctgaaaagc tgaacgagat ggttcccctc atcagccaag cctgcgacct |
| 721 | tctcctggct catttaaaac gctatgcgga atctggggac gcatttgaca tccagaggtg |
| 781 | ctactgcaat tacaccacag atgtggttgc cagcgtcgcc tttggcaccc cggtggactc |
| 841 | ctggcaggcc cctgaggatc cctttgtgaa acactgcaag cgtttcttcg aattctgcat |
| 901 | ccccagacct atcctggttt tactcttatc atttccatcc ataatggtcc cactggcccg |
| 961 | gattttgccc aataagaacc gagacgaact gaatggcttt tttaacaaac tcattaggaa |
| 1021 | tgtgattgcc ttgcgggacc agcaagctgc cgaagagagg cggagagact tcctccaaat |
| 1081 | ggtcctggat gcccgacatt ctgcaagtcc catgggcgtg caagactttg acatcgtcag |
| 1141 | agacgttttc tcctctactg ggtgcaagcc gaacccttcc cggcaacacc agcccagccc |
| 1201 | tatggccagg cctttgactg tggatgagat tgtgggccag gccttcatct tcctcatcgc |
| 1261 | tggctatgaa atcatcacca acacactttc ttttgccacc tacctactgg ccaccaaccc |
| 1321 | tgactgccaa gagaagcttc tgagagaggt agacgttttt aaggagaaac acatggcccc |
| 1381 | tgagttctgc agcctcgagg aaggcctgcc ctatctggac atggtgattg cagagacgct |
| 1441 | gaggatgtac ccgccagctt tcagattcac acgggaggca gctcaggact gcgaggtgct |
| 1501 | ggggcagcgc atccccgcag gcgctgtgct agagatggcc gtgggtgccc tgcaccatga |
| 1561 | ccctgagcac tggccaagcc cggagacctt caaccctgaa aggtaccgct gcagctagaa |
| 1621 | tccaaatctg ccctaggtcc aaaaaatggt gtctatatca agatcgtatc ccgctgacac |
| 1681 | agaaggctgc cgggtggggg gagggcaccc ccaaattcaa agaaaaccct aagtgtggat |
| 1741 | gttcagaatt ttggaaaaat gtcactgaag tgattgaaag agtgcctggc atgcaaggat |
| 1801 | aagaggttct ttacataaca tttcctaaat gcttaataaa cgtttgttgc acttggtttt |
| 1861 | gacattgcca atggggtttg aaccagtgct ctctctaaat gaaaaaaaaa aaaaaaaaa |

SEQ ID NO: 146 Human TRXAS1 Isoform 2 Amino Acid Sequence (NP_112246.2)

| | |
|---|---|
| 1 | mmealgflkl evngpmvtva lsvallallk wystsafsrl eklglrhpkp spfignltff |
| 61 | rqgfwesqme irklygplcg yylgrrmfiv isepdmikqv lvenfsnftn rmasglefks |
| 121 | vadsvlflrd krweevrgal msafspekln emvplisqac dlllahlkry aesgdafdiq |
| 181 | rcycnyttdv vasvafgtpv dswqapedpf vkhckrffef ciprpilvll lsfpsimvpl |
| 241 | arilpnknrd elngffnkli rnvialrdqq aaeerrrdfl qmvldarhsa spmgvqdfdi |
| 301 | vrdvfsstgc kpnpsrqhqp spmarpltvd eivgqafifl iagyeiitnt lsfatyllat |
| 361 | npdcqekllr evdvfkekhm apefcsleeg lpyldmviae tlrmyppafr ftreaaqdce |
| 421 | vigqripaga vlemavgalh hdpehwpspe tfnperyrcs |

SEQ ID NO: 147 Human TBXAS1 Transcript Variant 3 cDNA Sequence
(NM_001130966.2; CDS: 539-2143)

| | |
|---|---|
| 1 | gagggccggc ctggacccgg cgggcgcacg gctccgagcc cagcgcagcc gagccggtct |
| 61 | gcagaggggc ccccagaact ttgtgtgtgt gtgtgcacgc gcgcgcgtgt gtgcccaggt |
| 121 | gcgagggttc gcgcgtgtgc tggcggggag ggaaccgacc cgaaagagag ccccacgctg |
| 181 | gcaaagtggc tcgcccatct cacagccggt tggcgctcgc ggaggctcac agaacatttc |
| 241 | ctgcctgttg ctcacactct gttgtccaca gcatacccgc accctagatg gctgactctt |
| 301 | gtcttctgtc tctttatttt ccatgtgtga gcctgtgcct gtggtttatc tctggaggat |
| 361 | gtgcctgagg gaggagacat atactgcctg gactgcctgg ctgatgtgag ctagtttgtc |
| 421 | tggttgagtt ggatgtttaa atagaaggca gaacaacaac agagcacggt tcccataagg |
| 481 | gcggcgagat cagcctcctg tctcatctgg aagaccacca ctctggggtc tcagaggaat |
| 541 | gatggaagcc ttggggtttc taaaattgga agtgaatggc cccatggtga cggtggccct |
| 601 | gtcagtggct ctcttggccc tcctgaaatg gtactccaca tcagcattct caagactgga |
| 661 | gaagttaggc ctcagacatc ccaagccttc tcctttcatt ggaaacttga catttttccg |
| 721 | ccagggtttt tgggaaagcc aaatggagct cagaaagctg tatggacctc tgtgtgggta |
| 781 | ctatcttggt cgtcggatgt ttattgttat ttctgagcca gacatgatca agcaggtgtt |
| 841 | ggttgagaac ttcagtaact ttaccaacag aatggcgtcg ggtttggagt tcaagtcggt |
| 901 | agccgacagc gttctgtttt tacgtgacaa aagatgggaa gaggtcagag gtgccctgat |
| 961 | gtctgctttc agtcctgaaa agctgaacga gatggttccc ctcatcagcc aagcctgcga |
| 1021 | ccttctcctg gctcatttaa aacgctatgc ggaatctggg gacgcatttg acatccagag |
| 1081 | gtgctactgc aattacacca cagatgtggt tgccagcgtc gcctttggca ccccggtgga |
| 1141 | ctcctggcag gcccctgagg atccctttgt gaaacactgc aagcgtttct tcgaattctg |
| 1201 | catccccaga cctatcctgg ttttactctt atcatttcca tccataatgg tcccactggc |
| 1261 | ccggattttg cccaataaga accgagacga actgaatggc ttttttaaca aactcattag |
| 1321 | gaatgtgatt gccttgcggg accagcaagc tgccgaagag aggcggagag acttcctcca |
| 1381 | aatggtcctg gatgcccgac attctgcaag tcccatgggc gtgcaagact ttgacatcgt |
| 1441 | cagagacgtt ttctcctcta ctgggtgcaa gccgaaccct tcccggcaac accagcccag |
| 1501 | ccctatggcc aggcctttga ctgtggatga gattgtgggc caggccttca tcttcctcat |
| 1561 | cgctggctat gaaatcatca ccaacacact ttcttttgcc acctacctac tggccaccaa |
| 1621 | ccctgactgc caagagaagc ttctgagaga ggtagacgtt tttaaggaga aacacatggc |
| 1681 | ccctgagttc tgcagcctcg aggaaggcct gccctatctg gacatggtga ttgcagagac |
| 1741 | gctgaggatg tacccgccag ctttcagatt cacacgggag gcagctcagg actgcgaggt |
| 1801 | gctggggcag cgcatccccg caggcgctgt gctagagatg gccgtgggtg ccctgcacca |
| 1861 | tgaccctgag cactggccaa gcccggagac cttcaaccct gaaaggttca cggctgaggc |
| 1921 | ccggcagcag caccggccct tcacgtacct gcccttcggg gccggcccac ggagctgcct |
| 1981 | cggggtgcgt ctagggctgc ttgaggtcaa gttgacactg ctccacgtgc tgcacaagtt |
| 2041 | ccggttccaa gcctgccctg agacccaggt accgctgcag ctagaatcca aatctgccct |
| 2101 | aggtccaaaa aatggtgtct atatcaagat cgtatcccgc tgacacagaa ggctgccggg |
| 2161 | tggggggagg gcacccccaa attcaaagaa aaccctaagt gtggatgttc agaattttgg |
| 2221 | aaaaatgtca ctgaagtgat tgaaagagtg cctggcatgc aaggataaga ggttctttac |
| 2281 | ataacatttc ctaaatgctt aataaacgtt tgttgcactt ggttttgaca ttgccaatgg |
| 2341 | ggtttgaacc agtgctctct ctaaatgaaa aaaaaaaaaa aaaaa |

SEQ ID NO: 148 Human TBXAS1 Transcript Variant 4 cDNA Sequence
(NM_001166253.1; CDS: 236-1978)

| | |
|---|---|
| 1 | aaggaataaa gttgctgatt cattccttta cactgaaacc ctttgttgtg ccctcctctt |
| 61 | ccttcctctt tatagggaga cactctgaga aagagcacat tgtgggggcc cactccatgt |
| 121 | gatgtttgct tggttgcctg ttcccttttc tacctgcaga gcacggttcc cataagggcg |
| 181 | gcgagatcag cctcctgtct catctggaag accaccactc tggggtctca gaggaatgat |
| 241 | ggaagccttg gggtttctaa aattggaagt gaatggcccc atggtgacgg tggccctgtc |
| 301 | agtggctctc ttggccctcc tgaaatggta ctccacatca gcattctcaa gactggagaa |
| 361 | gttaggcctc agacatccca agccttctcc tttcattgga aacttgacat ttttccgcca |
| 421 | gggtttttgg gaaagccaaa tggagctcag aaagctgtat ggacctctgt gtgggtacta |
| 481 | tcttggtcgt cggatgttta ttgttatttc tgagccagac atgatcaagc aggtgttggt |
| 541 | tgagaacttc agtaacttta ccaacagaat ggcgtcgggt ttggagttca agtcggtagc |
| 601 | cgacagcgtt ctgtttttac gtgacaaaag atgggaagag gtcagaggtg ccctgatgtc |
| 661 | tgctttcagt cctgaaaagc tgaacgagct tggcctttta atcatgcaag agagaataaa |
| 721 | aggtcacatg ggtggccagc aggctccaca aagaattcca cccacccgtt tgtcgaagcc |
| 781 | gagtggtatc tatgtgaatc tccattatgc cactctacct ttctgtatgg ttcccctcat |
| 841 | cagccaagcc tgcgaccttc tcctggctca tttaaaacgc tatgcggaat ctggggacgc |
| 901 | atttgacatc cagaggtgct actgcaatta caccacagat gtggttgcca gcgtcgcctt |
| 961 | tggcaccccg gtggactcct ggcaggcccc tgaggatccc tttgtgaaac actgcaagcg |
| 1021 | tttcttcgaa ttctgcatcc ccagacctat cctggtttta ctcttatcat ttccatccat |
| 1081 | aatggtccca ctggcccgga ttttgcccaa taagaaccga gacgaactga atggcttttt |
| 1141 | taacaaactc attaggaatg tgattgcctt gcgggaccag caagctgccg aagagaggcg |
| 1201 | gagagacttc ctccaaatgg tcctggatgc ccgacattct gcaagtccca tgggcgtgca |
| 1261 | agactttgac atcgtcagag acgttttctc ctctactggg tgcaagccga acccttcccg |
| 1321 | gcaacaccag cccagcccta tggccaggcc tttgactgtg gatgagattg tgggccaggc |
| 1381 | cttcatcttc ctcatcgctg gctatgaaat catcaccaac acactttctt ttgccaccta |
| 1441 | cctactggcc accaaccctg actgccaaga gaagcttctg agagaggtag acgtttttaa |
| 1501 | ggagaaacac atggcccctg agttctgcag cctcgaggaa ggcctgccct atctggacat |
| 1561 | ggtgattgca gagacgctga ggatgtaccc gccagctttc agattcacac gggaggcagc |
| 1621 | tcaggactgc gaggtgctgg ggcagcgcat ccccgcaggc gctgtgctag agatggccgt |
| 1681 | gggtgccctg caccatgacc ctgagcactg gccaagcccg gagaccttca accctgaaag |
| 1741 | gttcacggct gaggcccggc agcagcaccg gcccttcacg tacctgccct tcggggccgg |
| 1801 | cccacggagc tgcctcgggg tgcgtctagg gctgcttgag gtcaagttga cactgctcca |
| 1861 | cgtgctgcac aagttccggt tccaagcctg ccctgagacc caggtaccgc tgcagctaga |
| 1921 | atccaaatct gccctaggtc caaaaaatgg tgtctatatc aagatcgtat cccgctgaca |
| 1981 | cagaaggctg ccgggtgggg ggagggcacc cccaaattca aagaaaaccc taagtgtgga |
| 2041 | tgttcagaat tttggaaaaa tgtcactgaa gtgattgaaa gagtgcctgg catgcaagga |
| 2101 | taagaggttc tttacataac atttcctaaa tgcttaataa acgtttgttg cacttggttt |
| 2161 | tgacattgcc aatggggttt gaaccagtgc tctctctaaa tgaaaaaaaa aaaaaaaaaa |

SEQ ID NO: 149 Human TBXAS1 Isoform 3 Amino Acid Sequence
(NM_001139725.1)

| | |
|---|---|
| 1 | mmealgflki evngpmvtva lsvallallk wystsafsrl eklgarhpkp spfignltff |
| 61 | rqgfwesqme lrklygplcg yylgrrmfiv isepdmikqv lvenfsnftn rmasglefks |
| 121 | vadsvlflrd krweevrgal msafspekln elgllimqer ikghmqgqqa pqripptrls |
| 181 | kpsgiyvnlh yatlpfcmvp lisqacdlll ahlkryaesg dafdiqrcyc nyttdvvasv |
| 241 | afgtpvdswq apedpfvkhc krffefcipr pilvlllsfp simvplaril pnknrdelng |
| 301 | ffnklirnvi alrdqqaaee rrrdflqmvl darhsaspmg vqdfdivrdv fsstgckpnp |
| 361 | srqhqpspma rpltvdeivg qafifliagy eiitntlsfa tyllatnpdc qekllrevdv |
| 421 | fkekhmapef csleeglpyl dmviaetlrm yppafrftre aaqdcevlgq ripagavlem |
| 481 | avgalhhdpe hwpspetfnp erftaearqq hrpftylpfg agprsclgvr lgllevkltl |
| 541 | lhvlhkfrfq acpetqvplq lesksalgpk ngvyikivsr |

SEQ ID NO: 150 Human TBXAS1 Transcript Variant 5 cDNA Sequence
(NM_001166254.1; CDS: 490-1890)

| | |
|---|---|
| 1 | gagggccggc ctggacccgg cgggcgcacg gctccgagcc cagcgcagcc gagccggtct |
| 61 | gcagaggggc ccccagaact ttgtgtgtgt gtgtgcacgc gcgcgcgtgt gtgcccaggt |
| 121 | gcgagggttc gcgcgtgtgc tggcggggag ggaaccgacc cgaaagagag ccccacgctg |
| 181 | gcaaagtggc tcgcccatct cacagccggt tggcgctcgc ggagctcttt attttccatg |
| 241 | tgtgagcctg tgcctgtggt ttatctctgg aggatgtgcc tgagggagga gacatatact |
| 301 | gcctggactg cctggctgat gtgagctagt ttgtctggtt gagttggatg tttaaataga |
| 361 | aggcagaaca acaacaggta ctccacatca gcattctcaa gactggagaa gttaggcctc |
| 421 | agacatccca agccttctcc tttcattgga aacttgacat ttttccgcca gggtttttgg |
| 481 | gaaagccaaa tggagctcag aaagctgtat ggacctctgt gtgggtacta tcttggtcgt |
| 541 | cggatgttta ttgttatttc tgagccagac atgatcaagc aggtgttggt tgagaacttc |
| 601 | agtaacttta ccaacagaat ggcgtcgggt ttggagttca agtcggtagc cgacagcgtt |
| 661 | ctgtttttac gtgacaaaag atgggaagag gtcagaggtg ccctgatgtc tgctttcagt |
| 721 | cctgaaaagc tgaacgagat ggttcccctc atcagccaag cctgcgacct tctcctggct |
| 781 | catttaaaac gctatgcgga atctggggac gcatttgaca tccagaggtg ctactgcaat |
| 841 | tacaccacag atgtggttgc cagcgtcgcc tttggcaccc cggtggactc ctggcaggcc |
| 901 | cctgaggatc cctttgtgaa acactgcaag cgtttcttcg aattctgcat ccccagacct |
| 961 | atcctggttt tactcttatc atttccatcc ataatggtcc cactggcccg gattttgccc |
| 1021 | aataagaacc gagacgaact gaatggcttt tttaacaaac tcattaggaa tgtgattgcc |
| 1081 | ttgcgggacc agcaagctgc cgaagagagg cggagagact tcctccaaat ggtcctggat |
| 7741 | gcccgacatt ctgcaagtcc catgggcgtg caagactttg acatcgtcag agacgttttc |
| 1201 | tcctctactg ggtgcaagcc gaacccttcc cggcaacacc agcccagccc tatggccagg |
| 1261 | cctttgactg tggatgagat tgtgggccag gccttcatct tcctcatcgc tggctatgaa |
| 1321 | atcatcacca acacactttc ttttgccacc tacctactgg ccaccaaccc tgactgccaa |
| 1381 | gagaagcttc tgagagaggt agacgttttt aaggagaaac acatggcccc tgagttctgc |
| 1441 | agcctcgagg aaggcctgcc ctatctggac atggtgattg cagagacgct gaggatgtac |
| 1501 | ccgccagctt tcagattcac acgggaggca gctcaggact gcgaggtgct ggggcagcgc |
| 1561 | atccccgcag gcgctgtgct agagatggcc gtgggtgccc tgcaccatga ccctgagcac |
| 1621 | tggccaagcc cggagacctt caaccctgaa aggttcacgg ctgaggcccg gcagcagcac |
| 1681 | cggcccttca cgtacctgcc cttcggggcc ggcccacgga gctgcctcgg ggtgcgtcta |
| 1741 | gggctgcttg aggtcaagtt gacactgctc cacgtgctgc acaagttccg gttccaagcc |
| 1801 | tgccctgaga cccaggtacc gctgcagcta gaatccaaat ctgccctagg tccaaaaaat |
| 1861 | ggtgtctata tcaagatcgt atcccgctga cacagaaggc tgccgggtgg ggggagggca |
| 1921 | cccccaaatt caaagaaaac cctaagtgtg gatgttcaga attttggaaa aatgtcactg |
| 1981 | aagtgattga aagagtgcct ggcatgcaag gataagaggt tctttacata acatttccta |
| 2041 | aatgcttaat aaacgtttgt tgcacttggt tttgacattg ccaatggggt ttgaaccagt |
| 2101 | gctctctcta aatgaaaaaa aaaaaaaaaa aa |

SEQ ID NO: 151 Human TBXAS1 Isoform 4 Amino Acid Sequence
(NM_001159726.1)

| | |
|---|---|
| 1 | melrklygpl cgyylgrrmf ivisepdmik qvlvenfsnf tnrmasglef ksvadsvlfl |
| 61 | rdkrweevrg almsafspek lnemvplisq acdlllahlk ryaesgdafd iqrcycnytt |
| 121 | dvvasvafgt pvdswqaped pfvkhckrff efciprpilv lllsfpsimv plarilpnkn |
| 181 | rdelngffnk lirnvialrd qqaaeerrrd flqmvldarh saspmqvqdf divrdvfsst |
| 241 | gckpnpsrqh qpspmarplt vdeivgqafi fliagyeiit ntlsfatyll atnpdcqekl |
| 301 | lrevdvfkek hmapefcsle eglpyldmvi aetlrmyppa frftreaaqd cevlgqripa |
| 361 | gavlemavga lhhdpehwps petfnperft aearqqhrpf tylpfgagpr sclgvrlgll |
| 421 | evkltllhvl hkfrfqacpe tqvplqlesk salgpkngvy ikivsr |

SEQ ID NO: 152 Human TBXAS1 Transcript Variant 6 cDNA Sequence
(NM_001314028.1; CDS: 325-1869)

| | |
|---|---|
| 1 | aaggaataaa gttgctgatt cattccttta cactgaaacc ctttgttgtg ccctcctctt |
| 61 | ccttcctctt tatagggaga cactctgaga aagagcacat tgtgggggcc cactccatgt |
| 121 | gatgtttgct tggttgcctg ttcccttttc tacctgcaga gcacggttcc cataagggcg |
| 181 | gcgagatcag cctcctgtct catctggaag accaccactc tggggtctca gaggaatgat |
| 241 | ggaagccttg gggtttctaa aattggaagt gaatggcccc atggtgacgg tggccctgtc |
| 301 | agtggctctc ttggccctcc tgaaatggta ctccacatca gcattctcaa gactggagaa |
| 361 | gttaggcctc agacatccca agccttctcc tttcattgga aacttgacat ttttccgcca |
| 421 | gggtttttgg gaaagccaaa tggagctcag aaagctgtat ggacctctgt gtgggtaaga |
| 481 | aggaaactca acgtttctat tatgtactat cttggtcgtc ggatgtttat tgttatttct |
| 541 | gagccagaca tgatcaagca ggtgttggtt gagaacttca gtaactttac caacagaatg |
| 601 | gcgtcgggtt tggagttcaa gtcggtagcc gacagcgttc tgtttttacg tgacaaaaga |
| 661 | tgggaagagg tcagaggtgc cctgatgtct gctttcagtc ctgaaaagct gaacgagatg |
| 721 | gttcccctca tcagccaagc ctgcgacctt ctcctggctc atttaaaacg ctatgcggaa |
| 781 | tctggggacg catttgacat ccagcggtgc tactgcaatt acaccacaga tgtggttgcc |
| 841 | agcgtcgcct ttggcacccc ggtggactcc tggcaggccc ctgaggatcc ctttgtgaaa |
| 901 | cactgcaagc gtttcttcga attctgcatc cccagaccta tcctggtttt actcttatca |
| 961 | tttccatcca taatggtccc actggcccgg attttgccca ataagaaccg agacgaactg |
| 1021 | aatggctttt ttaacaaact cattaggaat gtgattgcct tgcgggacca gcaagctgcc |
| 1081 | gaagagcggc ggagagactt cctccaaatg gtcctggatg cccgacattc tgcaagtccc |
| 1141 | atgggcgtgc aagactttga catcgtcaga gacgttttct cctctactgg gtgcaagccg |
| 1201 | aacccttccc ggcaacacca gcccagccct atggccaggc ctttgactgt ggatgagatt |
| 1261 | gtgggccagg ccttcatctt cctcatcgct ggctatgaaa tcatcaccaa cacactttct |
| 1321 | tttgccacct acctactggc caccaaccct gactgccaag agaagcttct gagagaggta |
| 1381 | gacgttttta aggagaaaca catggcccct gagttctgca gcctcgagga aggcctgccc |
| 1441 | tatctggaca tggtgattgc agagacgctg aggatgtacc cgccagcttt cagattcaca |
| 1501 | cgggaggcag ctcaggactg cgaggtgctg gggcagcgca tccccgcagg cgctgtgcta |
| 1561 | gagatggccg tgggtgccct gcaccatgac cctgagcact ggccaagccc ggagaccttc |
| 1621 | aaccctgcaa ggttcacggc tgaggcccgg cagcagcacc ggcccttcac gtacctgccc |
| 1681 | ttcggggccg gcccacggag ctgcctcggg gtgcgtctag ggctgcttga ggtccagttg |
| 1741 | acactgctcc acgtgctgca caagttccgg ttccaagcct gccctgagac ccaggtaccg |
| 1801 | ctgcagctag aatccaaatc tgccctaggt ccaaaaaatg gtgtctatat caagatcgta |
| 1861 | tcccgctgac acagaaggct gccgggtggg gggagggcac ccccaaattc aaagaaaacc |
| 1921 | ctaagtgtgg atgttcagca ttttggaaaa atgtcactga agtgattgaa agagtgcctg |
| 1981 | gcatgcaagg ataagaggtt ctttacataa catttcctaa atgcttaata aacgtttgtt |
| 2041 | gcacttggtt ttgacattgc caatggggtt tgaaccagtg ctctctctaa atgaaaaaaa |
| 2101 | aaaaaaaaaa a |

SEQ ID NO: 153 Human TBXAS1 Isoform 5 Amino Acid Sequence
(NP_001300957.1)

| | |
|---|---|
| 1 | mvlhisilkt gevrpqtsgq fsfhwkldif ppgflgkpng aqkavwtsvw vrrklnvsim |
| 61 | yylgrrmfiv isepdmikqv lvenfsnftn rmasglefks vadsvlflrd krweevrgal |
| 121 | msafspekln emvplisqac dlllahlkry aesgdafdiq rcycnyttdv vasvafgtpv |
| 181 | dswqapedpf vkhckrffef ciprpilvll lsfpsimvpl arilpnknrd elngffnkli |
| 241 | rnvialrdqq aaeerrrdfl qmvldarhsa spmgvqdfdi vrdvfsstgc kpnpsrqhqp |
| 301 | spmarpltvd eivgqafifl iagyeiitnt lsfatyllat npdcqekllr evdvfkekhm |
| 361 | apefcsleeg lpyldmviae tlrmyppafr ftreaaqdce vlgqripaga vlemavgalh |
| 421 | hdpehwpspe tfnperftae arqqhrpfty lpfgagprsc lgvrlgllev kltllhvlhk |
| 481 | frfqacpetq vplqlesksa lgpkngvyik ivsr |

SEQ ID NO: 154 Mouse TBXAS1 cDNA Sequence (NM_011539.3; CDS: 168-1769)

| | |
|---|---|
| 1 | gcacactcta agaaagagcd cttttggggg actcacttga tgggatgttc agctgactac |
| 61 | ctacttcttt ctccaccacc tatatacaga tcaggactcc tataagggca ggagagcagc |
| 121 | accctctttc ctcctgagga ccacccctcc ggggtctccg aggaaaaatg gaagtgttgg |
| 181 | ggcttctcaa gtttgaagtc agtggtacca tagtgactgt gactctgctc gtggctctct |
| 241 | tggccctcct gaaatggtac tccatgtcag ctttctcaag actggagcag ttgggcatca |
| 301 | ggcaccccaa gccttctcct tttgttggaa acttgatgtt tttccgccag ggtttttggg |
| 361 | agagccaatt ggaactccga gagcgatacg ggcctctgtg tgggtactat cttggccgtc |
| 421 | ggatgcacgt tgtcatttca gagccagaca tgatcaagca ggtgttggtt gagaacttca |
| 481 | gtaacttttc caacagaatg gcctcaggtc tggaacccaa gatggtagca gacagtgtcd |
| 541 | tgttgttacg tgacagaaga tgggaggaag tcaggggtgc cctgatgtct tcattcagtc |
| 601 | ctgaaaagtt ggatgagatg acacctctca tcagccaagc ctgtgaactt ctcgtggctc |
| 661 | acttaaaacg ctatgcagca tccagggacg cattcaacat ccagaggtgt tactgctgtt |
| 721 | acaccataga tgtggtggcc agtgtggcct ttggcaccca ggtggactcc cagaattctc |
| 781 | cagaagatcc ctttgtgcaa cactgccggc gtgcttccac cttctgtatc cccaggcctc |
| 841 | tcctggtatt aatcttatca tttccatcca taatggtccc attggcccgg attctgccca |
| 901 | ataagaaccg agatgaactg aatggctttt ttaacacact cattaggaat gtgattgcct |
| 961 | tacgggacca gcaagcagca gaagagaggc ggagagactt cctgcagatg gtgctggatg |
| 1021 | cccagcactc catgaactct gtgggcgtgg aaggctttga catggtccca gaatccctgt |
| 1081 | cctcttctga gtgcacaaag gaaccacccc aaaggtgcca tcctacctcc acatctaagc |
| 1141 | ctttcactgt ggatgaaatt gtgggccagg ccttcctctt cctcattgcg ggccatgagg |
| 1201 | tcatcacaaa cacgctgtcc ttcatcacat acctgctggc cacccaccct gactgccagg |
| 1261 | agaggcttct gaaagaggtg gacctcttca tggggaagca cccagcccct gagtaccaca |
| 1321 | gcctgcagga aggtctgccg tatctggaca tggtgatttc agagaccctg aggatgtacc |
| 1381 | caccagcttt caggttcaca cgggaggcag cacaggactg tgaggtgctg ggacaacgta |
| 1441 | tccctgcagg tacagtgctg gagatagctg tgggtgccct acaccatgac ccagagcact |
| 1501 | ggccgaatcc tgagaccttt gaccctgaaa ggttcacagc agaggcccgg cttcagcgga |
| 1561 | ggccgttcac atacctgccc tttggagctg gccccaggag ctgcctcgga gtgcggctgg |
| 1621 | gcctgctggt ggtcaagctg acaatactcd aggtcctaca caagttccgc tttgaagcca |
| 1681 | gccctgagac tcaggttcca cttcagctag aatccaaatc tgccctaggc cccaaaaatg |
| 1741 | gagtctacat caagattgtg tcacgctgat atagaaggca cgtgggaaag aggaagcact |
| 1801 | ttgtaatggt aacatctgca catcgctgca ggagggcacc cgggattcag atgaaagcct |
| 1861 | cgtgtggaga tggagagttt ataggaatat atatcttagg gtgatcaaaa gggcacatgg |
| 1921 | catgtgagcc tactttgtca caattcctaa atgtttaata aatgtttacc atgcttcaaa |
| 1981 | aaaaaaaaaa aa |

SEQ ID NO: 155 Mouse TBXAS1 Amino Acid Sequence (NP_035669.3)

| | |
|---|---|
| 1 | mevlgllkfe vsgtivtvtl lvallallkw ysmsafsrle klgirhpkps pfvgnlmffr |
| 61 | qgfwesqlel rerygplcgy ylgrrmhvvi sepdmikqvl venfsnfsnr masglepkmv |
| 121 | adsvlllrdr rweevrgalm ssfspeklde mtplisqace llvahlkrya asrdafniqr |
| 181 | cyccytidvv asvafgtqvd sqnspedpfv qhcrrastfc iprpllvlil sfpsimvpla |
| 241 | rilpnknrde lngffntlir nvialrdqqa aeerrrdflq mvldaqhsmn svgvegfdmv |
| 301 | peslsssect keppqrchpt stskpftvde ivgqaflfli aghevitntl sfityllath |
| 361 | pdcqerllke vdlfmgkhpa peyhslqegl pyldmviset lrmyppafrf treaaqdcev |
| 421 | lgqripagtv leiavgalhh dpehwpnpet fdperftaea rlqrrpftyl pfgagprscl |
| 481 | gvrlgllvvk ltilqvlhkf rfeaspetqv plqlesksal gpkngvyiki vsr |

SEQ ID NO: 156 Human SIGLEC7 Transcript Variant 1 cDNA Sequence
(NM_014385.3; CDS: 70-1473)

| | |
|---|---|
| 1 | gcatttcctg agagaagaac cctgaggaac agacgttccc tcgcggccct ggcacctcca |
| 61 | accccagata tgctgctgct gctgctgctg cccctgctct gggggaggga gagggtggaa |
| 121 | ggacagaaga gtaaccggaa ggattactcg ctgacgatgc agagttccgt gaccgtgcaa |
| 181 | gagggcatgt gtgtccatgt gcgctgctcc ttctcctacc cagtggacag ccagactgac |
| 241 | tctgacccag ttcatggcta ctggttccgg gcagggaatg atataagctg gaaggctcca |
| 301 | gtggccacaa acaacccagc ttgggcagtg caggaggaaa ctcgggaccg attccacctc |
| 361 | cttggggacc cacagaccaa aaattgcacc ctgagcatca gagatgccag aatgagtgat |
| 421 | gcggggagat acttctttcg tatggagaaa ggaaatataa aatggaatta taaatatgac |
| 481 | cagctctctg tgaacgtgac agccttgacc cacaggccca acatccttat ccccggtacc |
| 541 | ctggagtctg gctgcttcca gaatctgacc tgctctgtgc cctgggcctg tgagcagggg |
| 601 | acgcccccta tgatctcctg gatggggacc tctgtgtccc ccctgcaccc ctccaccacc |
| 661 | cgctcctcag tgctcaccct catcccacag ccccagcacc acggcaccag cctcacctgt |
| 721 | caggtgacct tgcctggggc cggcgtgacc acgaacagga ccatccaact caatgtgtca |
| 781 | taccctcctc agaacttgac tgtgactgtc ttccaaggag aaggcacagc atccacagct |
| 841 | ctggggaaca gctcatctct ttcagtccta gagggccagt ctctgcgctt gctctgtgct |
| 901 | gttgacagca atccccctgc caggctgagc tggacctgga ggagtctgac cctgtacccc |
| 961 | tcacagccct caaaccctct ggtactggag ctgcaagtgc acctggggga tgaaggggaa |
| 1021 | ttcacctgtc gagctcagaa ctctctgggt tcccagcacg tttccctgaa cctctccctg |
| 1081 | caacaggagt acacaggcaa aatgaggcct gtatcaggag tgttgctggg ggcggtcggg |
| 1141 | ggcgctggag ccacagccct ggtcttcctc tccttctgtg tcatcttcat tgtagtgagg |
| 1201 | tcctgcagga agaaatcggc aaggccagch gcggacgtgg gagacatagg catgaaggat |
| 1261 | gcaaacacca tcaggggctc agcctctcag ggtaacctga ctgagtcctg ggcagatgat |
| 1321 | aacccccgac accatggcct ggctgcccac tcctcagggg aggaaagaga gctccagtat |
| 1381 | gcacccctca gctttcataa gggggagcct caggacctat caggacaaga agccaccaac |
| 1441 | aatgagtact cagagatcaa gatccccaag taagaaaatg cagaggctcg ggcttgtttg |
| 1501 | agggttcacg acccctccag caaaggagtc tgaggctgat tccagtagaa ttagcagccc |
| 1561 | tcaatgctgt gcaacaagac atcagaactt attcctcttg tctaactgaa aatgcatgcc |
| 1621 | tgatgaccaa actctccctt tccccatcca atcggtccac aatccccgac ctggcctctg |
| 1681 | gtacccacca ttctcctctg tacttctcta aggatgacta ctttagattc cgaatatagt |
| 1741 | gagattgtaa cgtgaaaaaa aaaaaaaaa |

SEQ ID NO: 157 Human SIGLEC7 Isoform 1 Amino Acid Sequence (NP_055200.1)

| | |
|---|---|
| 1 | mllllllpll wgrervegqk snrkdysltm gssvtvqegm cvhvrcsfsy pvdsqtdsdp |
| 61 | vhgywfragn diswkapvat nnpawavqee trdrfhllgd pqtknctlsi rdarmsdagr |
| 121 | yffrmekgni kwnykydqls vnvtalthrp nilipgtles gcfqnltcsv pwaceqgtpp |
| 181 | miswmgtsvs plhpsttrss vltlipqpqh hgtsltcqvt lpgagvttnr tiqlnysypp |
| 241 | qnltvtvfqg egtastalgn ssslsvlegq slrlvcavds npparlswtw rsltlypsgp |
| 301 | snplvlelqv hlgdegeftc raqnslgsqh vslnlslqqe ytgkmrpvsg vllgavggag |
| 361 | atalvflsfc vifivvrscr kksarpaadv gdigmkdant irgsasqgnl teswaddnpr |
| 421 | hhglaahssg eereiqyapl sfhkgepqdl sgqeatnney seikipk |

SEQ ID NO: 158 Human SIGLEC7 Transcript Variant 2 cDNA Sequence
(NM_016543.3; CDS: 70-1194)

| | |
|---|---|
| 1 | gcagttcctg agagaagaac cctgaggaac agacgttccc tcgcggccct ggcacctcca |
| 61 | accccagata tgctgctgct gctgctgctg cccctgctct gggggaggga gagggtggaa |
| 121 | ggacagaaga gtaaccggaa ggattactcg ctgacgctgc agagttccgt gaccgtgcaa |
| 181 | gagggcatgt gtgtccatgt gcgctgctcc ttctcctacc cagtggacag ccagactgac |
| 241 | tctgacccag ttcatggcta ctggttccgg gcagggcatg atataagctg gcaggctcca |
| 301 | gtggccacaa acaacccagc ttgggcagtg caggaggaaa ctcgggaccg attccacctc |
| 361 | cttggggacc cacagaccaa aaattgcacc ctgagcatca gagatgccag aatgagtgat |
| 421 | gcggggagat acttctttcg tatggagaaa ggcaatataa aatggaatta taaatatgac |
| 481 | cagctctctg tgaacgtgac agaccctcct cagaacttga ctgtgactgt cttccaagga |
| 541 | gaaggcacag catccacagc tctggggaac agctcatctc tttcagtcct agagggccag |
| 601 | tctctgcgct tggtctgtgc tgttgacagc aatccccctg ccaggctgag ctggacctgg |
| 661 | aggagtctga ccctgtaccc ctcacaggcc tcaaaccctc tggtactgga gctgcaagtg |
| 721 | cacctggggg atgaagggga attcacctgt cgagctcaga actctctggg ttcccagcac |
| 781 | gtttccctga acctctccct gcaacaggag tacacaggca aaatgaggcc tgtatcagga |
| 841 | gtgttgctgg gggcggtcgg gggagctgga gccacagccc tggtcttcct ctccttctgt |
| 901 | gtcatcttca ttgtagtgag gtcctgcagg aagaaatcgg caaggccagc agcggacgtg |
| 961 | ggagacatag gcatgaagga tgcaaacacc atcaggggct cagcctctca gggtaacctg |
| 1021 | actgagtcct gggcagatga taacccccga caccatggcc tggctgccca ctcctcaggg |
| 1081 | gaggaaagag agatccagta tgcacccctc agctttcata agggggagcc tcaggaccta |
| 1141 | tcaggacaag aagccaccaa caatgagtac tcagagatca agatccccaa gtaagaaaat |
| 1201 | gcagaggctc gggcttgttt gagggttcac gacccctcca gcaaaggagt ctgaggctga |
| 1261 | ttccagtaga attagcagcc ctcaatgctg tgcaacaaga catcagaact tattcctctt |
| 1321 | gtctaactga aaatgcatgc ctgatgacca aactctccct ttccccatcc aatcggtcca |
| 1381 | cactccccgc cctggcctct ggtacccacc attctcctct gtacttctct aaggatgact |
| 1441 | actttagatt ccgaatatag tgagattgta acgtgaaaaa aaaaaaaaaa |

SEQ ID NO: 159 Human SIGLEC7 Isoform 2 Amino Acid Sequence (NP_057627.2)

| | |
|---|---|
| 1 | mllllllpll wgrervegqk snrkdysltm qssvtvqegm cvhvrcsfsy pvdsqtdsdp |
| 61 | vhgywfragn diswkapvat nnpawavqee trdrfhllgd pqtknctlsi rdarmsdagr |
| 121 | yffrmekgni kwnykydqls vnvtdppqnl tvtvfqgegt astalgnsss lsvlegqslr |
| 181 | lvcavdsnpp arlswtwrsl tlypsqpsnp lvlelqvhlg degeftcraq nslgsqhvsl |
| 241 | nlslqqeytg kmrpvsgvll gavggagata lvflsfcvif ivvrscrkks arpaadvgdi |
| 301 | gmkdantirg sasqgnltes waddnprhhg laahssgeer eiqyaplsfh kgepqdlsgq |
| 361 | eatnneysei kipk |

SEQ ID NO: 160 Human SIGLEC7 Transcript Variant 3 cDNA Sequence
(NM_001277201.1; CDS: 70-507)

| | |
|---|---|
| 1 | gcagttcctg agagaagaac cctgaggaac agacgttccc tcgcggccct ggcacctcca |
| 61 | accccagata tgctgctgct gctgctgctg cccctgctct gggggaggga gagggtggaa |
| 121 | ggacagaaga gtaaccggaa ggattactcg ctgacgatgc agagttccgt gaccgtgcaa |
| 161 | gagggcatgt gtgtccatgt gcgctgctcc ttctcctacc cagtggacag ccagactgac |
| 241 | tctgacccag ttcatggcta ctggttccgg gcagggaatg atataagctg gaaggctcca |
| 301 | gtggccacaa acaacccagc ttgggcagtg caggaggaaa ctcgggaccg attccacctc |
| 361 | cttggggacc cacagaccaa aaattgcacc ctgagcatca gagatgccag aatgagtgat |
| 421 | gcggggagat acttctttcg tatggagaaa ggaaatataa aatggaatta taaatatgac |
| 481 | cagctctctg tgaacgtgac agggtaacct gactgagtcc tgggcagatg ataacccccg |
| 541 | acaccatggc ctggctgccc actcctcagg ggaggaaaga gagatccagt atgcacccct |
| 601 | cagctttcat aagggggagc ctcaggacct atcaggacaa gaagccacca acaatgagta |
| 661 | ctcagagatc aagatcccca agtaagaaaa tgcagaggct cgggcttgtt tgagggttca |
| 721 | cgacccctcc agcaaaggag tctgaggctg attccagtag aattagcagc cctcaatgct |
| 761 | gtgcaacaag acatcagaac ttattcctct tgtctaactg aaaatgcatg cctgatgacc |
| 641 | aaactctccc tttccccatc caatcggtcc acactccccg ccctggcctc tggtacccac |
| 901 | cattctcctc tgtacttctc taaggatgac tactttagat tccgaatata gtgagattgt |
| 961 | aacgtgaaaa aaaaaaaaaa a |

SEQ ID NO: 161 Human SIGLEC7 Isoform 3 Amino Acid Sequence
(NP_001264130.1)

| | |
|---|---|
| 1 | mllllllpll wgrervegqk snrkdysltm qssvtvqegm cvhvrcsfsy pvdsqtdsdp |
| 61 | vhgywfragn diswkapvat nnpawavqee trdrfhllgd pqtknctlsi rdarmsdagr |
| 121 | yffrmekgni kwnykydqls vnvtg |

SEQ ID NO: 162 Human DOCK2 Amino Acid Sequence (NP_004937.1)

| | |
|---|---|
| 1 | mapwrkadke rhgvaiynfq gsgapqlslq igdvvriqet cgdwyrgyli khkmaqgifp |
| 61 | ksfihikevt vekrrnteni ipaeiplaqe vtttlwewgs iwkqlyvask kerflqvqsm |
| 121 | mydlmewrsq llsgtlpkde lkelkqkvts kidygnkile ldlivrdedg nildpdntsv |
| 181 | islfhaheea tdkiterike emskdqpdya mysrissspt hslyvfvrnf vcrigedael |
| 241 | fmslydpnkq tvisenylvr wgsrgfpkei emlnnlkvvf tdlgnkdlnr dkiylicqiv |
| 301 | rvgkmdlkdt gakkctqglr rpfgvavmdi tdiikgkaes deekqhfipf hpvtaendfl |
| 361 | hsllgkvias kgdsggqglw vtmkmlvgdi iqirkdyphl vdrttvvark lgfpeiimpg |
| 421 | dvrndiyitl lqgdfdkynk ttqrnvevim cvcaedgktl pnaicvgsgd kpmneyrsvv |
| 461 | yyqvkqprwm etvkvavpie dmqrihlrfm frhrsslesk dkgeknfams yvklmkedgt |
| 541 | tlhdgfhdlv vlkgdskkme dasayltlps yrhhvenkga tlsrssssvg glsvssrdvf |
| 601 | sistlvcstk ltqnvgllgl lkwrmkpqll qenleklkiv dgeevvkflq dtldalfnim |
| 661 | mehsqsdeyd ilvfdaliyi igliadrkfq hfntvleayi qqhfsatlay kklmtvlkty |
| 721 | ldtssrgeqc epilrtlkal eyvfkfivrs rtlfsqlyeg keqmefeesm rrlfesinnl |
| 781 | mksqykttil lqvaalkyip svlhdvemvf dakllsqlly efytcippvk lqkqkvqsmn |
| 841 | eivqsnlfkk qecrdillpv itkelkelle qkddmqhqvl erkycvelln silevlsyqd |
| 901 | aaftyhhiqe imvqllrtvn rtvitmgrdh ilishfvacm tailnqmgdq hysfyietfq |
| 961 | tsselvdflm etfimfkdli gknvypgdwm amsmvqnrvf lrainkfaet mnqkflehtn |
| 1021 | fefqlwnnyf hlavafitqd slqleqfsha kynkilnkyg dmrrligfsi rdmwyklgqn |
| 1061 | kicfipgmvg pilemtlipe aelrkatipi ffdmmlceyq rsgdfkkfen eiilkldhev |
| 1141 | eggrgdeqym qllesilmec aaehptiaks venfvnlvkg lleklldyrg vmtdeskdnr |
| 1201 | msctvnllnf ykdnnreemy irylyklrdi hldcdnytea aytlllhtwl lkwsdeqcas |
| 1261 | qvmqtgqqhp qthrqlketl yetiigyfdk gkmweeaisl ckelaeqyem eifdyellsq |
| 1321 | nliqqakfye simkilrpkp dyfavgyygq gfpsflrnkv fiyrgkeyer redfqmqlmt |
| 1381 | qfpnaekmnt tsapgddvkn apgqyiqcft vqpvldehpr fknkpvpdqi infyksnyvq |
| 1441 | rfhysrpvrr gtvdpenefa smwiertsfv tayklpgilr wfevvhmsqt tisplenaie |
| 1501 | tmstanekil mminqyqsde tlpinplsml lngivdpavm ggfakyekaf fteeyvrdhp |
| 1561 | edqdklthlk dliawqipfl gagikihekr vsdnlrpfhd rmeecfknlk mkvekeygvr |
| 1621 | empdfddrrv grprsmlrsy rqmsiislas mnsdcstpsk ptsesfdlel aspktprveq |
| 1661 | eepispgstl pevklrrskk rtkrssvvfa dekaaaesdl krlsrkhefm sdtnasehaa |
| 1741 | iplkasvlsq msfasqsmpt ipalalsvag ipgldeants prlsqtflql sdgdkktltr |
| 1801 | kkvnqffktm lasksaeegk qipdslstdl |

SEQ ID NO: 163 Human DOCK2 cDNA Sequence (NM_004946.3; CDS: 53-5545)

| | |
|---|---|
| 1 | agccaccccc tgacggcttc cccacgggag gacgcgaggc cccggcccag ccatggcccc |
| 61 | ctggcgcaaa gctgacaagg agcggcacgg cgtggccata tacaacttcc aaggcagcgg |
| 121 | agccccccag ctctccctgc agatcggcga tgtggtgcga atacaggaga cgtgtggaga |
| 181 | ctggtatagg ggatacctca taaagcacaa aatgttacag ggcatttttc ctaagtcatt |
| 241 | tatccacatc aaggaagtga cagttgagaa aagaagaaat actgagaaca tcattcctgc |
| 301 | agaaattcct ctggcacaag aagtgacaac gacactttgg gaatggggaa gcatctggaa |
| 361 | acaactctat gtggccagca aaaaggagcg ttttctccag gtgcagtcca tgatgtacga |
| 421 | tctgatggag tggaggtccc agcttctctc aggaacctta cccaaggatg agctgaagga |
| 481 | actgaagcag aaagtcacgt ccaaaattga ctatggcaac aaaatccttg agcttgattt |
| 541 | gattgtcaga gatgaagacg gaaatatctt ggaccctgat aataccagtg tcatcagctt |
| 601 | gttccatgca catgaggaag caactgataa aatcacagag cgtatcaaag aagaaatgtc |
| 661 | aaaagaccag ccagattatg caatgtattc ccggatctcc tcatccccca cccatagcct |
| 721 | ctatgtgttt gtgagaaact ttgtgtgcag aattggggaa gatgctgagc tcttcatgtc |
| 781 | tctctacgac cccaacaagc aaacggtcat aagtgagaac tacctagtgc gatggggcag |
| 841 | ccggggcttc cctaaggaga ttgagatgct caacaatctg aaggtggtct tcacggatct |
| 901 | tggaaacaaa gacctcaaca gggataaaat ttacttgatt tgtcaaatag tccgggtcgg |
| 961 | caagatggat cttaaggata ctggtgcaaa gaagtgcacg cagggactga ggaggccctt |
| 1021 | tggggtggca gttatggata taacagacat catcaagggg aaagcagaga gtgatgaaga |
| 1081 | aaagcagcac ttcattcctt ttcacccggt tacagctgag aatgacttcc tacacagcct |
| 1141 | gctgggcaaa gtcatagcct ccaaggggga cagtggaggg caaggcctct gggtgaccat |
| 1201 | gaagatgctg gtgggtgaca tcattcagat tcgcaaggac tatccacacc tggtggacag |
| 1261 | gaccaccgtg gtggccagga agctgggatt cccagagatc atcatgccag gggatgtcag |
| 1321 | gaacgacatc tacattactc tcttacaagg tgactttgac aagtacaaca agaccacaca |
| 1381 | gaggaatgtg gaagtcatca tgtgtgtgtg cgcggaggat ggcaaaacgc tgcctaatgc |
| 1441 | aatttgcgtg ggagcagggg acaagcccat gaatgagtat cgctccgttg tgtactatca |
| 1501 | agtcaaacag ccacgctgga tggaaacagt caaggtggct gtccctattg aagacatgca |
| 1561 | gaggatccat ctgcgattca tgtttcgaca tcggtcatct ctggaatcta aagataaagg |
| 1621 | agaaaagaac tttgccatgt cctatgtgaa gctgatgaaa gaagatggga ctactctaca |
| 1681 | cgatggattc catgacttag ttgtcctcaa gggggacagc aagaagatgg aggatgccag |
| 1741 | cgcatacctg acccttcctt cttatcgaca ccatgtggaa aacaaggggg ccacgctgag |
| 1801 | caggagctcc agcagtgttg gggggctttc tgtcagctcc cgggatgtgt tctccatttc |
| 1861 | caccctggtg tgctccacaa agctcactca gaatgtgggc ttgctgggtt tgctgaagtg |
| 1921 | gcgtatgaag cctcaactgc tacaggagaa tttagaaaag ttgaagattg tggatggaga |
| 1981 | ggaagtggtg aagtttctcc aggatactct ggatgccctc ttcaacatca tgatggagca |
| 2041 | ttctcaaagt gatgaatatg acatcctcgt ctttgatgcc ttgatttaca taataggact |
| 2101 | cattgcagac cggaaatttc agcatttcaa caccgttctg gaggcttaca tccaacagca |
| 2161 | tttcagtgcg accttggctt acaagaaatt gatgacagtg ctgaagactt acttggatac |
| 2221 | ctccagcaga ggggagcaat gtgagccaat cctaagaacg ctgaaggctt tggaatatgt |
| 2281 | gttcaagttc attgttcggt cgaggacatt attttcacag ctttatccag gcaaagaaca |
| 2341 | gatggagttt gaagaatcca tgagacggct ctttgaatcc atcaacaatc tgatgaaaag |
| 2401 | tcaatacaaa actaccatcc ttttgcaggt ggcggctttg aaatacatcc catctgtcct |
| 2461 | gcatgatgta gaaatggtct ttgatgcgaa gttactcagc caactcctgt atgagttcta |
| 2521 | cacctgcatc cctcctgtga aactccagaa gcagaaagta cagtctatga atgagatagt |
| 2581 | ccagagcaac ctctttaaaa agcaagaatg ccgggacatt ctgcttcctg tcatcaccaa |
| 2641 | agagctgaag gagctgctgg agcagaagga tgacatgcaa caccaggtcc tggagaggaa |
| 2701 | gtactgcgtt gaattgctca acagcatctt ggaagtcctt agctaccagg atgcggcctt |
| 2761 | cacctaccac catatccagg agatcatggt ccagctgctg cggacagtga accggacagt |
| 2821 | catcaccatg ggcggggatc acattctgat tagtcacttt gtggcatgta tgacagccat |
| 2881 | cttaaaccag atgggtgacc agcactactc cttctacatt gagaccttcc agaccagctc |
| 2941 | tgaacttgtg gacttcttga tggagacctt catcatgttc aaggacctca ttggaaagaa |
| 3001 | cgtgtaccct ggagactgga tggccatgag catggttcaa aacagggtct tcctgagagc |
| 3061 | tatcaacaag tttgcagaaa ccatgaacca gaagttccta gaacacacga actttgagtt |
| 3121 | ccagctgtgg aacaactatt ttcatctggc agtggctttt atcaccgarm attctctgca |
| 3181 | gctggagcag ttctcacacg ccaaatacaa caaaatcctg aataagtatg gggacatgag |
| 3241 | acggctaatt ggcttctcca tccgtgatat gtggtacaag cttggtcaga acaaaatctg |
| 3301 | cttcatccca ggcatggtag gacctatatt agagatgaca cttatccctg aggctgagct |
| 3361 | ccggaaagcc accataccaa tcttcttcga catgatgctg tgtgaatatc aaagaagtgg |
| 3421 | ggatttaact aagtttgaaa acgaaatcat cctgaagctg gaccacgagg tagaaggggg |
| 3481 | ccgaggcgac gagcagtaca tgcagctcct ggagtcaatc ctgatggaat gtgctgcaga |
| 3541 | gcacccaacc attgccaagt cggtggagaa cttcgtgaac ctggtcaaag gcctcctgga |
| 3601 | gaagctgctg gattaccggg gtgtgatgac agatgagagc aaagacaacc gcatgagctg |
| 3661 | caccgtgaac ctgctgaatt tctacaaaga taacaacagg gaggagatgt acataaggta |
| 3721 | cctgtacaaa ctccgcgatc ttcacctgga ctgtgacaat tacacagagg ctgcctacac |
| 3781 | gctccttctc cacacctggc ttctcaagtg gtcggatgag cagtgtgcat cacaggtcat |
| 3841 | gcagacaggc cagcagcacc cccagacaca ccggcagctg aaggagacgc tctacgagac |
| 3901 | catcataggc tactttgaca aaggaaagat gtgggaagag gccataagtc tgtgcaagga |
| 3961 | gctggcggaa cagtacgaga tggagatctt tgactatgag ctgctcagcc agaacctgat |
| 4021 | ccagcaggca aaattctatg aaagcatcat gaaaatcctc aggcccaaac cagactactt |
| 4081 | tgctgttgga tactacggcc agggattccc ctccttcctg cggaacaaag tgttcatcta |
| 4141 | ccgcgggaag gaatatgagc gaagagaaga tttccagatg cagctgatga cccagttccc |
| 4201 | caatgcagag aagatgaaca ccacctctgc cccgggagat gatgtgaaga atgccccagg |
| 4261 | ccagtatatc cagtgcttca ctgtccagcc tgtcttggat gaacatccca ggttcaagaa |
| 4321 | taagccagtg cctgaccaga ttataaactt ctacaaatcc aactacgtgc aaaggttcca |
| 4381 | ctactcccgg cccgtgcgca gggggaccgt agacccagag aatgagtttg cttcgatgtg |
| 4441 | gattgagaga acctccttcg tgactgcata caagctgccg gggatcctgc gctggtttga |
| 4501 | ggtggtgcac atgtcgcaga ccacaattag tcctctggag aatgccatag aaaccatgtc |
| 4561 | cacggccaat gagaagatcc tgatgatgat aaaccagtac cagagtgatg agaccctccc |
| 4621 | catcaaccca ctctccatgc tcctgaacgg gattgtggac cctgctgtca tgggaggctt |
| 4681 | cgccaagtat gagaaggcct tcttcactga agagtatgtc agggaccacc ctgaggacca |
| 4741 | ggacaagctg acccacctca aggacctgat tgcatggcag atccccttct tgggagctgg |
| 4801 | gattaagatc catgagaaaa gggtgtcaga taacttgcga cccttccatg accggatgga |
| 4861 | ggaatgtttc aagaacctga aaatgaaggt ggagaaggag tacggtgtcc gagagatgcc |
| 4921 | tgactttgac gacaggagag tgggccgtcc caggtctatg ctgcgctcat acagacagat |
| 4981 | gtccatcatc tctctggctt ccatgaattc tgactgcagc acccccagca agcctacctc |
| 5041 | agagagcttt gacctggaat tagcatcacc caagacgccg agagtggagc aggaggaacc |
| 5101 | gatctccccg gggagcaccc tgcctgaggt caagctgcgg aggtccaaga agaggacaaa |
| 5161 | gagaagcagc gtagtttttg cggatgagaa agcagctgca gagtcggacc tgaagcggct |
| 5221 | ttccaggaag catgagttca tgagtgacac caacctctcg gagcatgcgg ccatccccct |
| 5281 | caaggcgtct gtcctctctc aaatgagctt tgccagccag tccatgccta ccatcccagc |
| 5341 | cctggcgctc tcagtggcag gcatccctgg gttggatgag gccaacacat ctccccgcct |
| 5401 | cagccagacc ttcctccaac tctcagatgg tgacaagaag acactcacac ggaagaaggt |
| 5461 | caatcagttc ttcaagacaa tgctggccag caaatcggct gaagaaggca aacagatccc |
| 5521 | agactcgctg tccacggacc tgtgagctgc tgctgactag ggctgcatgg gagagccagg |
| 5581 | gaggggagtt tctggaagag ga.aagccat cgtggaacat cgaagcctca gagagtggga |
| 5641 | gactgtcccc atcagttgtc cttacttaga ggagacagag aggccaatca ggtcccagag |
| 5701 | cttgaatgct aacaagccca gcatcccctg gggctgtgat catggtggat gaggaagcct |
| 5761 | caacgtagat tcctgaactc aaggtaccag caagaatgcc ttctcccagt gtgctctccc |
| 5821 | caacatccta ggcacagctt tcataaccca gtttcttagg tgtaagaaac tgtttttatc |
| 5881 | tcatttatta agtctcagaa cttaacagaa aaggaagcct tttaaatatt ctttttaatt |
| 5941 | ttattttaga ttaacagttt tgtactttac atttttttat acaaccaacc agtttctttt |
| 6001 | ctagccaatc atctctgaag agttgctgtt tcttactgac aataaaaaat gttctcttgg |
| 6061 | ttcgaataa |

SEQ ID NO: 164 Mouse DOCK2 Amino Acid Sequence (NP_203538.2)

| | |
|---|---|
| 1 | mapwrktdke rhgvaiynfq gseaqhltlq igdvvriqet cgdwyrgyli khklsqgifp |
| 61 | tsfihlkevt vekrtnieni ipaeiplaqe vtttlwewgs iwkqlyvask kerflqvqsm |
| 121 | mydlmewrsq llsgtlpkde lkelkqkvts kidygnkile ldlivrdedg nildpdktsv |
| 181 | islfhaheea tykiterike emskdqpdyg vysrissspt hslyvfvrnf vcrigedael |
| 241 | fmslydphkq tvisenylvr wgskgfpkei emlnnlkvvf tdlgnkdlnr dkiflicqiv |
| 301 | rigkmdlkdi nakkctqglr rpfgvavmdi tdiikgkaes deekghfipf hpvsaendfl |
| 361 | hsllgkvias kgdsggqglw vtmkmlvgdi iqirkdyphl vdrttvvark lgfpeiimpg |
| 421 | dvrndiyitl lqgdfdkytk ttqrnvevim cvctedgkvl pnaicvgagd kamneyhsvv |
| 481 | yyqvkqprwm etvkvavpie dmqrihlrfm frhrsslesk dkgeknfams yvklmkedgt |
| 541 | tlhdgyhelv vlkgdskkme dasayltlps yrhpvenkga tlsrssssvg glsvssrdvf |
| 601 | sistlvcstk ltqnvgllgl lkwrmkpqll qenleklkiv dgeevvkflq dtldalfnim |
| 661 | mehsqsneyd ilvfdaliyi igliadrkfq hfntvleayi qqhfsatlay kklmtvlkty |
| 721 | ldtssrgeqc epilrtlkal eyvfkfivrs rtlfsqlyeg keqmefeesm rrlfesinnl |
| 781 | mksqykttil lqvaalkyip svlhdvetvf dakllsqlly efytcippvk lqkqkvqsmn |
| 841 | eivqsnlfkk qecrdillpv itkelkelle qrddgqhqae kkhcvellns ilevlscqda |
| 901 | aftydhiqei mvqllrtvnr tvitmgrdha lishfvacmt aildqmgdqh ysfyietfqt |
| 961 | ssdlvdflme tfimfkdlig knvypgdwma msmvqnrvfl rainkfaetm nqkflehtsf |
| 1021 | efqlwnnyfh lavafitqds lqleqfthak ynkilnkygd mrrligfsir dmwyklgqnk |
| 1081 | icfipgmvgp ilemtlipea elrkatipif fdmmlceyqr tgafkkfene iilkldheve |
| 1141 | ggrgdeqymq llesilmect aehptiaksv enfvslvkgl leklldyrgv mtdeskdnrm |
| 1201 | sctvnllnfy kdnnreemyi rylyklrdlh ldcenyteaa ytlllhtwll kwsdeqcasq |
| 1261 | vmqtgqqnpq thrqlketly etiigyfdkg kmweeaislc kelaeqyeme ifdyellsqh |
| 1321 | ltqqakfyen imkilrtkpd yfavgyygqg fpsflrnkvf iyrgkeyerr edfqmqllsq |
| 1381 | fpnaekmntt sapgddvrna pgqyiqcftv qpvldehprf knkpvpdqii nfyksnvvqk |
| 1441 | fhysrpvrrg kvdpenefas mwiertsflt ayklpgilrw fevvhmsqtt isplenaiet |
| 1501 | mstvnekilm minqyqsdes lpinplsmll ngivdpavmg gfakyekaff teeysrehpe |
| 1561 | dqdklshlkd liawqipflg agikihekrv sdnlrpfhdr meecfknlkm kvekeygvre |
| 1621 | mpdfedrrvg rprsmlrsyr qmsvislasm hsdcstpskv paesfdlesa ppktpkveee |
| 1681 | pispgstlpe vklrrskkrt krssvvfade kaatesdlkr lsrkqefmsd tnlsehaaip |
| 1741 | arvsilsqms fasqsmptip altlsvagvp gldeantspr lsqtffqvsd gdkktlkkkk |
| 1801 | vnqffktmla sksseeskqi pdflstnm |

SEQ ID NO: 165 Mouse DOCK2 cDNA Sequence (NM_033374.3; CDS: 83-5569)

| | |
|---|---|
| 1 | agtggggccc tgcaaggcgc ctaaccaccc cagccagctt cttcactaga gaacaggagg |
| 61 | ctctcaaggc tccggtcttg ccatggcccc ctggcgcaaa actgacaagg agcgtcacgg |
| 121 | agtggctatc tacaacttcc aaggcagtga aggccagcat ctcacgctac agattggcga |
| 181 | tgtggtacga atacaggaga cctgtggaga ctggtacaga gggtacctca taaagcataa |
| 241 | actgtcacag ggcattttcc ctacatcctt tatccatctc aaggaagtga cagtggagas |
| 301 | gagaaggaac atagagaaca tcattcctgc agaaatccct ctggcacaag aagtgacaac |
| 361 | cacgctctgg gagtggggaa gcatctggaa gcagctctat gtggccagca aaaaggaacg |
| 421 | cttcctccaa gtgcagtcca tgatgtacga cctgatggaa tggcgctctc agctcctctc |
| 481 | aggaacgcta cccaaggatg agctgaagga actgaagcag aaagtcacat cgaagatcga |
| 541 | ctatggcaac aaastccttg agctcgatct cattgtcaga gatgaagatg gaaacatact |
| 601 | ggaccctgat aagaccagcg tcatcagctt gttccatgct cacgaggagg caacttacaa |
| 661 | aatcacagag cgcatcaaag aagaaatgtc aaaagaccag ccagattatg gggtttattc |
| 721 | tcggatctcc tgatccccca cccacagcct ctacgtcttt gtgagaaact ttgtgtgccg |
| 781 | aattggggag gatgctgagc tcttcatgtc tctctatgac cctcacaagc aaacggtcat |
| 841 | cagtgagaac tatctggtgc gatggggcag caaaggattc ccaaaggaaa ttgagatgct |
| 901 | caataacctg aaggtagtct tcacggatct tggaaataaa gacctcaaca gggataagat |
| 961 | tttcttgatc tgtcaaatag tccgaattgg gaagatggat cttaaggaca ttaatgccaa |
| 1021 | gaagtgcacc caggggctga ggagaccttt tggggtggca gttatggaca tcaccgacat |
| 1081 | catcaagggc aaggcagaga gcgacgaaga gaagcaacat tttattccat ttcacccggt |
| 1141 | ctcagctgag aacgatttcc ttcacagcct tctgggcaaa gtcatagctt ccaagggaga |
| 1201 | cagtggaggg caaggccttt gggtgaccat gaagatgctg gtgggggaca tcattcagat |
| 1261 | ccgtaaagac tacccacact tggtggacag gaccactgtg gtagcccgaa agctgggctt |
| 1321 | cccagagatc atcatgccag gggatgtcag gaacgacatc tacatcactc tcttacaagg |
| 1381 | tgactttgac aagtacacca agaccacaca gcggaacgtg gaagtgatca tgtgtgtgtg |
| 1441 | cacagaggat ggcaaagtgg tacctaatgc aatttgtgtg ggagcggggg ataaggccat |
| 1501 | gaacgagtac cactcagtcg tctactacca agtcaaacag ccccgatgga tggaaacagt |
| 1561 | caaggtggct gtccctattg aagatatgca gaggatccat ttgaggttca tgtttcgaca |
| 1621 | ccgctcatca ctagaatcta aagataaagg agaaaagaac tttgccatgt cctacgtgaa |
| 1681 | actcatgaaa gaagatggaa ccactctgca tgatggatac cacgagttgg tggttctcaa |
| 1741 | gggagacagc aagaaaatgg aagatgccag cgcttacctg acacttcctt cttatcgaca |
| 1801 | ccccgtggaa aacaagggag ctacgctgag ccggaggtcc agcagtgttg ggggcctttc |
| 1861 | tgtcagctcc cgggatgtgt tctccatttc cacgctagtg tgctccacaa agctgaccca |
| 1921 | gaatgtgggt ttgcttggct tgctgaagtg gcgtatgaag ccacaactgc tccaggagaa |
| 1981 | tctagagaag ttgaagattg tggatggcga gccagtagtg aagttcctcc aggacactct |
| 2041 | ggatgccctc ttcaacatca tgatggaaca ctctcagagt aacgagtatg acatcctcgt |
| 2101 | ctttgatgct ttgatctata taataggact cattgcagac cggaaattcc agcattttaa |
| 2161 | caccgtattg gaggcttaca tccaacagca tttcagtgct accttggcct acaagaaact |
| 2221 | gatgacggtg ctgaagactt acttggatac ctccagcagg ggggagcagt gcgagcccat |
| 2281 | cctcagaacg ctcaaagcct tagaatacgt gttcaagttc attgttcggt cgaggacatt |
| 2341 | attctcacag ctttacgaag ggaaagagca gatggagttt gaagaatcca tgagacggct |
| 2401 | cttcgaatcc atcaacaacc tgatgaaaag tcagtacaag accaccatcc tattgcaggt |
| 2461 | ggcggctttg aaatacatcc cttcagtcct tcacgatgtg gaaacagtct ttgatgccaa |
| 2521 | gttgctcagc cagctactgt atgagttcta cacctgcatc cctcccgtga agctgcagaa |
| 2581 | gcagaaagtt cagtccatga atgagatcgt gcagagcaac ctcttcaaaa agcaagaatg |
| 2641 | ccgggacatt ctgcttcctg tcatcaccaa agagctgaag gagctgctgg agcagaggga |
| 2701 | cgatgggcag caccaggctg agaagaagca ctgtgtggaa cttctcaaca gcatcttgga |
| 2761 | ggtcctcagc tgtcaggatg cggccttcac ttatgaccac atccaagaga tcatggtcca |
| 2821 | gctgcttcgc acagtgaacc ggacagtcat cacgatgggc cgagatcacg ctttgattag |
| 2881 | tcactttgtg gcgtgtatga cagctatctt agaccagatg ggcgaccaac actattcatt |
| 2941 | ttacattgag accttccaga ccagctcaga ccttgtggac ttcttgatgg agacattcat |
| 3001 | catgttcaag gacctcattg ggaagaatgt gtatcctggg gactggatgg ccatgagcat |
| 3061 | ggtccagaac cgggtcttcc tgagagccat caacaagttt gcagaaacca tgaaccagaa |
| 3121 | gtttctggaa cacacaagct ttgagttcca gctgtggaac aactattttc atctggcagt |
| 3181 | tgcctttatc actcaggact ctctgcagct ggagcagttc acacacgcta agtacaacaa |
| 3241 | aatcctgaat aagtacgggg acatgagacg gctcatagga ttctccatcc gtgatatgtg |
| 3301 | gtacaagctt ggccagaaca aaatctgctt catcccgggc atggtgggac ctatattaga |
| 3361 | gatgacactt atccctgagg ctgagctcag gaaagccacc atcccaatct tcttcgacat |
| 3421 | gatgctgtgt gaatatcaaa ggactggggc tttcaaaaag tttgaaaatg aaatcatcct |
| 3481 | gaagctggac catgaggtgg aaggaggccg gggggatgag cagtacatgc aactgctgga |
| 3541 | gtccatcctg atggagtgca cagcggaaca cccaaccatt gccaagtcgg tggagaactt |
| 3601 | tgtgagcttg gtcaaagggc tgctggagaa gttgctagat taccggggcg tgatgacaga |
| 3661 | tgagagcaaa gacaaccgta tgagctgcac agtgaacttg ctgaatttct acaaagataa |
| 3721 | caaccgggaa gagatgtaca taagatacct gtataaactg cgtgaccttc acttggactg |
| 3781 | tgaaaactac acagaggctg cctacacact cctcctccac acctggcttc tcaagtggtc |
| 3841 | agatgagcag tgtgcatccc aggtcatgca gacaggccag cagcaccctc agacccaccg |
| 3901 | gcagctgaag gagacactct atgagaccat tataggctac tttgacaaag gaaagatgtg |
| 3961 | ggaagaggcc atcagcctgt gcaaggaact ggcggaacaa tatgagatgg agatctttga |
| 4021 | ctacgagcta ctcagccaga acctgaccca gcaggccaaa ttctatgaaa acatcatgaa |
| 4081 | aatcctcaga accaaaccag actactttgc tgttggatac tatggccagg gattcccctc |
| 4141 | ctttctgcgg aacaaagtgt tcatctaccg aggcaaggaa tatgagcgaa gagaagactt |
| 4201 | ccagatgcag cttttgagcc agttccccaa cgcagaaaag atgaacacaa cttctgcccc |
| 4261 | aggagacgat gtgaggaatg ccccaggcca gtacatccag tgtttcactg tgcagcctgt |
| 4321 | tctggatgaa caccccaggt tcaagaacaa accggtgcct gaccagatca taaactttta |
| 4381 | caagtctaat tatgtgcaaa agttccacta ctccaggcct gtgcgcaggg gcaaggtaga |
| 4441 | cccagagaac gagtttgctt ccatgtggat cgagaggact tccttcctga cggcctacaa |
| 4501 | gctgcctggc atcctgcgct ggtttgaggt agttcacatg tctcagacca caattagtcc |
| 4561 | tctggagaat gccatcgaga ctatgtccac agtcaacgag aagatcctga tgatgataaa |
| 4621 | ccagtaccag agtgatgaaa gcctccccat caaccctctc tccatgctcc tcaatgggat |
| 4681 | cgtggaccct gctgtcatgg gaggcttcgc caagtatgag aaggccttct tcactgaaga |
| 4741 | gtatagcagg gagcacccgg aggaccagga caagctgagc catctcaagg acctgattgc |
| 4801 | atggcagatc cctttcctgg gagctgggat taagatccac gagaaaaggg tgtcagacaa |
| 4861 | cctgcgcccc ttccatgacc ggatggagga gtgcttcaag aacctgaaaa tgaaggtgga |
| 4921 | aaaggagtat ggtgtccgag agatgcctga cttcgaagac aggagagtgg gccgccccag |
| 4981 | gtccatgctg cgttcctaca ggcagatgtc cgtcatctct ctggcctcca tgcattctga |
| 5041 | ctgcagcact cccagcaaag tccccgcaga aagttttgac ctagagtcag ccccacccaa |
| 5101 | gactcccaaa gtggaggagg agcccatctc cccagggagc actctgccag aggtcaagct |
| 5161 | gcggcggtcc aagaaaagga ccaagaggag cagcgtggtg tttgcagacg agaaggcagc |
| 5221 | aacggagtca gacctgaagc ggctttctag aaagcaagag ttcatgagtg acaccaacct |
| 5281 | ctcagagcat gcagccatcc ctgccagggt gtctatcctc tctcagatga gttttgccag |
| 5341 | ccagtccatg cccaccatcc cagccctcac actctccgtg gcaggcgtgc ctgggttgga |
| 5401 | tgaggccaac acatctcccc gcctgagtca gaccttcttc caagtctcag atggtgacaa |
| 5461 | gaagaccctg aaaaagaaga aagtcaatca attcttcaag accatgctgg ctagcaagtc |
| 5521 | gagtgaagaa agcaagcaga tcccagactt cctgtccacc aacatgtgag gcactgctga |
| 5581 | gccgagcact gtgggaactc gagcgggaat gcatgagtat gaccttggag ttccacaagg |
| 5641 | agacaagtct gtaaaagaga cagatgatct caaggggccc ccagaactca agtgatgact |
| 5701 | agccttgcat ctcctgtggc cactacaaca gtgatggatg actcaacaga caaagaagcc |
| 5761 | ccaccatgga ttgttggacc ccaaggagcc agcaagtgcc caatgaatgt tctccccagc |
| 5821 | atccaaggga cgcatgtcat aactcaaatt cacagctagg aggagctgtc ttctgtttca |
| 5881 | ttcctaagtg tcagcactta aaagaactga tttaaatgtt ctattttccc ttaacatttt |
| 5941 | tgtactttta catttttttt ctaataagcc ccttgctact ccatcatctc cagactgctg |
| 6001 | ctctttctta cccacattaa aaatgaccca tagctcaaat gaaccttgtt gctgagtctc |
| 6061 | ttattgaagg actgaggaga aagatcccag gatgggaggc agcccagggg actacagcaa |
| 6121 | tatgaaaggc ttatgttggc tcatctcaat gccatgtttt ctctccttgc tcaggcaaac |
| 6181 | tcaagaatct tctctgctct aagaaagaag caggaatgag atttcttcta atttctccat |
| 6241 | tgtttagtta ttttgatttg agatacttta ctgatataca agctatttaa actatttggg |
| 6301 | gaaaaaagca atgtttgaga tagctaatat aatggcttct caatggaatg atagcacaat |
| 6361 | ttgaaatgat accaaaaaga ataataaaat gaaccgtttg aggtttgctt aaaaaaaaaa |
| 6421 | aaaaaaaaaa a |

*The nucleic acid and polypeptide sequences of the biomarkers encompassed by the present invention listed in Table 1 have been submitted at GenBank under the unique identifier provided herein and each such uniquely identified sequence submitted at GenBank is hereby incorporated in its entirety by reference.
*Included in Table 1 are RNA nucleic acid molecules (e.g., thymidines replaced with uridines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any publicly available sequence listed in Table 1 (see below for example), or a portion thereof Such nucleic acid molecules can have a function of the full-length nucleic acid as described further herein.
*Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 97%, 93%, 94%, 95%, 96%, 97%, 98%, 99%, 99.5%, or more identity across their full length with an amino acid sequence of any publicly available sequence listed in and Table I (see below for example), or a portion thereof. Such polypeptides can have a function of the full-length polypeptide as described further herein.
*Included in Table 1 are additional known nucleic acid and amino acid sequences for the listed biomarkers.

TABLE 2
CD53
FERMT3
CD37
CXorf21
CD48
CD84
SEQ ID NO: 166 Human CD53 Transcript Variant 1 cDNA Sequence
(NM_001040033.1; CDS: 172-831)

| | |
|---|---|
| 1 | gaggacagac tgaagaaaca tccaaggtgg tcttgaagga cactgggatc ctgtaacaca |
| 61 | gccccggata tctgtgttac cagccttgtc tcggccacct caaggataat cactaaattc |
| 121 | tgccgaaagg actgaggaac ggtgcctgga aaagggcaag aatatcacgg catgggcatg |
| 181 | agtagcttga aactgctgcc gtatgtcctg tttttcttca acttgctctt ttggatctgt |
| 241 | ggctgctgca ttttgggctt tgggatctac ctgctgatcc acaacaactt cggagtgctc |
| 301 | ttccataacc tcccctccct cacgctgggc aatgtgtttg tcatcgtggg ctctattatc |
| 361 | atggtagttg ccttcctggg ctgcatgggc tctatcaagg aaaacaagtg tctgcttatg |
| 421 | tcgttcttca tcctgctgct gattatcctc cttgctgagg tgaccttggc catcctgctc |
| 481 | tttgtatatg aacagaagct gcctgagtat gtggctaagg gtctgaccga cagcatccac |
| 541 | cgttaccact cagacaatag caccaaggca gcgtgggact ccatccagtc atttctgcag |
| 601 | tgttgtggta taaatggcac gagtgattgg accagtggcc caccagcatc ttgcccctca |
| 661 | gatcgaaaag tggagggttg ctatgcgaaa gcaagactgt ggtttcattc caatttcctg |
| 721 | tatatcggaa tcatcaccat ctgtgtatgt gtgattgagg tgttggggat gtcctttgca |
| 781 | ctgaccctga actgccagct tgacaaaacc agccagacca tagggctatg atctgcagta |
| 841 | gtcctgtggt gaagagactt gtttcatctc cggaaatgca aaaccattta tagcatgaag |
| 901 | ccctacatga tcactgcagg atgatcctcc tcccatcctt tcccttttta ggtccctgtc |
| 961 | ttatacaacc agagcagtgg gtgttggcca ggcacatccc atctcaggca gcaagacaat |
| 1021 | ctttcactca ctgacggcag cagccatgtc tctcaaagtg gtgcaactaa tatctgagca |
| 1081 | tcttttagac aagagaggca aagacaaact ggatttaatg gcccaacatc aaagggtgaa |
| 1141 | cccaggatat gaatttttgc atcttcccat tgtcgaatta gtctccagcc tctaaataat |
| 1201 | gcccagtctt ctccccaaag tcaagcaaga gactagttga agggagttct ggggccaggc |
| 1261 | tcactggacc attgtcacaa ccctctgttt ctctttgact aagtgccctg gctacaggaa |
| 1321 | ttacacagtt ctctttctcc aaagggcaag atctcatttc aatttcttta ttagagggcc |
| 1381 | ttattgatgt gttctaagtc tttccagaaa aaaactatcc agtgatttat atcctgattt |
| 1441 | caaccagtca cttagctgat aatcacagta agaagacttc tggtattatc tctctatcag |
| 1501 | ataagatttt gttaatgtac tattttactc ttcaataaat aaaagtttat tatctcaatc |
| 1561 | acaacattgc ta |

SEQ ID NO: 167 Human CD53 Isoform 1 Amino Acid Sequence (NP_000551.1 and
NP_001035122.1)

| | |
|---|---|
| 1 | mgmsslkllk yvlfffnllf wicgccilgf giyllihnnf gvlfhnlpsl tlgnvfvivg |
| 61 | siimvvaflg cmgsikenkc llmsffilll iillaevtla illfvyeqkl neyvakgltd |
| 121 | sihryhsdns tkaawdsiqs flqccgingt sdwtsgppas cpsdrkvegc yakarlwfhs |
| 181 | nflyigiiti cvcvievlgm sfaltlncqi dktsqtigl |

SEQ ID NO: 168 Human CD53 Transcript Variant 2 cDNA Sequence (NM_000560.3;
CDS: 167-826)

| | |
|---|---|
| 1 | gtcgtcacag catgatcata ttttttcacc cttcacttct ccttttacac aaatagcccc |
| 61 | ggatatctgt gttaccagcc ttgtctcggc cacctcaagg ataatcacta aattctgccg |
| 121 | aaaggactga ggaacggtgc ctggaaaagg gcaagaatat cacggcatgg gcatgagtag |
| 181 | cttgaaactg ctgaagtatg tcctgttttt cttcaacttg ctcttttgga tctgtggctg |
| 241 | ctgcattttg ggctttggga tctacctgct gatccacaac aacttcggag tgctcttcca |
| 301 | taacctcccc tccctcacgc tgggcaatgt gtttgtcatc gtgggctcta ttatcatggt |
| 361 | agttgccttc ctgggctgca tgggctctat caaggaacac aagtgtctgc ttatgtcgtt |
| 421 | cttcatcctg ctgctgatta tcatccttgc tgaggtgacc ttggccatcc tgctctttgt |
| 481 | atatgaacag aagctgaatg agtatgtggc taagggtctg accgacagca tccaccgtta |
| 541 | ccactcagac aatagcacca aggcagcgtg ggactccatc cagtcatttc tgcagtgttg |
| 601 | tggtataaat ggcacgagtg attggaccag tggcccacca gcatcttgcc cctcagatcg |
| 661 | aaaagtggag ggttgctatg cgaaagcaag actgtggttt cattccaatt tcctgtatat |
| 721 | cggaatcatc accatctgtg tatgtgtgat tgaggtgttg gggatgtcct ttgcactgac |
| 781 | cctgaactgc cagattgaca aaacgagcca gaccataggg ctatgatctg cagtagtcct |
| 841 | gtggtgaaga gacttgtttc atctccggaa atgcaaaacc atttatagca tgaagcccta |
| 901 | catgatcact gcaggatgat cctcctccca tcctttccct ttttaggtcc ctgtcttata |
| 961 | caaccagaga agtgggtgtt ggccaggcac atcccatctc aggaagcaag acaatctttc |
| 1021 | actcactgac ggcagcagcc atgtctctca aagtggtgaa actaatatct gagcatcttt |
| 1081 | tagacaagag aggcaaagac aaactggatt taatggccca acatcaaagg gtgaacccag |
| 1141 | gatatgaatt tttgcatctt cccattgtcg aattagtctc cagcctctaa ataatgccca |
| 1201 | gtcttctccc caaagtcaag caagagacta gttgaaggga gttctggggc caggctcact |
| 1261 | ggaccattgt cacaaccctc tgtttctctt tgactaagtg ccctggctac aggaattaca |
| 1321 | cagttctctt tctccaaagg gcaagatctc atttcaattt ctttattaga gggccttatt |
| 1381 | gatgtgttct aagtctttcc agaaaaaaac tatccagtga tttatatcct gatttcaacc |
| 1441 | agtcacttag ctgataatca cagtaagaag acttctggta ttatctctct atcagataag |
| 1501 | attttgttaa tgtactattt tactcttcaa taaataacag tttattatct caatcacaac |
| 1561 | attgcta |

SEQ ID NO: 169 Human CD53 Isoform 2 Amino Acid Sequence (NP_001307567.1)

| | |
|---|---|
| 1 | mgmsslkllk yvlfffnllf wicgccilgf giyllihnnf gvlfhnlpsl tlgnvfvivg |
| 61 | siimvvaflg cmgsikenkc llmsffilll iillaevtla illfvyeqkg cyakarlwfh |
| 121 | snflyigiit icvcvievlg msfaltlncq idktsqtigl |

SEQ ID NO: 170 Human CD53 Transcript Variant 3 cDNA Sequence
(NM_001320638.1; CDS: 167-649)

| | |
|---|---|
| 1 | gtcgtcacag catgatcata ttttttcacc cttcacttct ccttttacac aaatagcccc |
| 61 | ggatatctgt gttaccagcc ttgtctcggc cacctcaagg ataatcacta aattctgccg |
| 121 | aaaggactga ggaacggtgc ctggaaaagg gcaagaatat cacggcatgg gcatgagtag |
| 181 | cttgaaactg ctgaagtatg tcctgttttt cttcaacttg ctcttttgga tctgtggctg |
| 241 | ctgcattttg ggctttggga tctacctgct gatccacaac aacttcggag tgctcttcca |
| 301 | taacctcccc tccctcacgc tgggcaatgt gtttgtcatc gtgggctcta ttatcatggt |
| 361 | agttgccttc ctgggctgca tgggctctat caaggaaaac aagtgtctgc ttatgtcgtt |
| 421 | cttcatcctg ctgctgatta tcctccttgc tgaggtgacc ttggccatcc tgctctttgt |
| 481 | atatgaacag aagggttgct atgcgaaagc aagactgtgg tttcattcca atttcctgta |
| 541 | tatcggaatc atcaccatct gtgtatgtgt gattgaggtg ttggggatgt cctttgcact |
| 601 | gaccctgaac tgccagattg acaaaaccag ccagaccata gggctatgat ctgcagtagt |
| 661 | cctgtggtga agagacttgt ttcatctccg gaaatgcaaa accatttata gcatgaagcc |
| 721 | ctacatgatc actgcaggat gatcctcctc ccatcctttc cctttttagg tccctgtctt |
| 781 | atacaaccag agaagtgggt gttggccagg cacatcccat ctcaggcagc aagacaatct |
| 841 | ttcactcact gacggcagca gccatgtctc tcaaagtggt gaaactaata tctgagcatc |
| 901 | ttttagacaa gagaggcaaa gacaaactgg atttaatggc ccaacatcaa agggtgaacc |
| 961 | caggatatga atttttgcat cttcccattg tcgaattagt ctccagcctc taaataatgc |
| 1021 | ccagtcttct ccccaaagtc aagcaagaga ctagttgaag ggagttctgg ggccaggctc |
| 1081 | actggaccat tgtcacaacc ctctgtttct ctttgactaa gtgccctggc tacaggaatt |
| 1141 | acacagttct ctttctccaa agggcaagat ctcatttcaa tttctttatt agagggcctt |
| 1201 | attgatgtgt tctaagtctt tccagaaaaa aactatccag tgatttatat cctgatttca |
| 1261 | accagtcact tagctgataa tcacagtaag aagacttctg gtattatctc tctatcagat |
| 1321 | aagattttgt taatgtacta ttttactctt caataaataa aagtttatta tctcaatcac |
| 1381 | aacattgcta |

SEQ ID NO: 171 Mouse CD53 cDNA Sequence (NM_007651.3; CDS: 200-859)

| | |
|---|---|
| 1 | agtctcactt cctcactctt ctcgcttggg tttcctgtcg tcacagcatg attgtatttt |
| 61 | ttctctcttc acttctcctt ttacacaaat agacatagac ttctgggtta caggctgtgc |
| 121 | tggccaccta aaagataatc agtgaattct acctgaagta ctgagggaca ctgccttcaa |
| 181 | aagggcatac tatcccagca tgggcatgag cagcctgaaa ttgctgaaat atgttctgtt |
| 241 | tatctttaac ttgctttttt gggtctgtgg ctgttgcatt ttgggctttg gcatctattt |
| 301 | cctggtccaa aatacctatg gagtactctt ccgtaacctt cccttcctga cacttggcaa |
| 361 | cattctggtc attgtgggat ccattatcat ggtagttgcc ttcttgggtt gcatgggctc |
| 421 | aatcaaggaa aataagtgcc tgcttatgtc gttctttgtt ctgctgctga ttattctcct |
| 481 | tgctgaggtg accatagcca tcctgctctt tgtgtatgaa caaaaactca acactttagt |
| 541 | ggctgagggt ctgaatgaca gcatccaaca ttatcactct gacaacagca ctatgcaggc |
| 601 | atgggacttc atccagacac aactgcagtg ttgtggtgta aatggctcaa gtgattggac |
| 661 | cagtggtcca ccatcttcct gcccatcagg tgcagatgtt cagggttgct ataataaggc |
| 721 | aaaatcgtgg tttcactcca atttcttgta tattggaatc attaccatct gtgtatgtgt |
| 781 | gatacaggtg ctgggaatgt cctttgcact gacactcaac tgccagattg acaaaacaag |
| 841 | ccaggcttta gggctgtgac ttgcaacttc cccctgctta agtgacttat tcctctctag |
| 901 | aaagtcaaag catccattcc atgagaactt aaacaattac ctgcctgact ggcattttgg |
| 961 | cttcttctta ttccatcttt gactggatct ctgtcttata cacatccact gaagagaata |
| 1021 | tttgtcatgg acttcccata tcaagcagaa gacaaacatt aaccaactga tagcagtaac |
| 1081 | catatccctt aaagatggtg aaacatacct gggtgttttt ggtttttttt ttttttttac |
| 1141 | atacttgggt atttttttta aagagacact gtagcactgg tggcttgaga tccactgcca |
| 1201 | gctgctggtg tggttatttc tcagagtact agcctagcaa atgtgagccc ttgagtttag |
| 1261 | ccccaaatac tacaaaaaag aggtccaagt ttaaatgtta gtctcctaac aactgtcaaa |
| 1321 | tcaatttcta gcctctaaat cttgctactt ccactctaca aagtcacata agagagaagc |
| 1381 | tgatggaaat ttttgagtcc cattcattag ataattgaca tactcagttt ccttttgaac |
| 1441 | acagtccttg gtaataggaa tcatacagaa atcttttatt tctggaaaat attccaatLt |
| 1501 | ctttgtctta ttgattttgt tccatccatc catccagaaa agattattcc catcctattg |
| 1561 | ttagtcagtc tggtagcctt gaattacatt gccataaaac aacccagaag tattaatatc |
| 1621 | tccagtgtgt tagctgataa tcacatccat gtctatgttt tatttctcta ttaaataagg |
| 1681 | ttctgttaat gtaccatttt aacctgttaa taaacaaaag tttataatca ctatgcatca |
| 1741 | aacatttgac tcattctgat atttctgtta caagccaaat atatgttata tttctgtata |
| 1801 | acaaattagt tcaaaatgta gtggctaaga acctatatat ttgttttata aattgactga |
| 1861 | tttaaagaca gcattttgct atgtagccca ggctgtcttg gaacttacta tatgaccaaa |
| 1921 | gatggcacca aactcatgat taccctgctt ttaacttctg aatattagaa ttacagttaa |
| 1981 | gtgctactaa gtcagaacaa acatttattg tctgatggct ttatgtaatc aggagtgtgg |
| 2041 | aagtagctta gaaaaatgat tatagatagt tgtttcttgc atatagtgat taagtggtca |
| 2101 | gccatatctt ccatcatctc atgtctcatt ggaaaattaa tccattgtca tgttctcata |
| 2161 | tagttgtaat gaacctcata taatggccac acataagttt atatgggtag tttcgtgaat |
| 2221 | aatgagacag gtaagacata aaatgatagg tagcatccaa gacaaaagtc atagcacttt |
| 2281 | tataacttaa cccagaaatg atggcatcag ttattctggg tcaaaagttc aatttccacc |
| 2341 | cagggcaaaa ttacacaaca ggcatatgac ttctaggaag ttgagatcat tgatggccac |
| 2401 | atgagagatt gctaaccatc tcatgcttac tatgtcaaaa aatatggtgt acttgtctat |
| 2461 | attatgtata tacataacta tattctataa agtaaaaaat tagaacacat ataatgccta |
| 2521 | atttatagaa ctcacaagaa tagaaaataa ttggtttttg ttttagaaat gttctgtaga |
| 2581 | attctctgac ttgctttaag gaaaactggt tattttcgtg gtgctctata gggaaaatat |
| 2641 | ctattgcatt ttctgagtca tataaagagt catgtattcc tctttgttca gactaacact |
| 2701 | tagtggtgac attaccagta agttttcctg cctaatgcct gagctttgtt cttagctcta |
| 2761 | ctttgttctt cactcccaat aaaatgttat ggagttctaa gg |

SEQ ID NO: 172 Mouse CD53 Amino Acid Sequence (NP_031677.1)

| | |
|---|---|
| 1 | mgmsslkllk yvlfifnllf wvcgccilgf giyflvqnty gvlfrnlpfl tlgnilvivg |
| 61 | siimvvaflg cmgsikenkc llmsffvlll iillaevtia illfvyeqkl ntlvaeglnd |
| 121 | siqhyhsdns tmkawdfiqt qlqccgvngs sdwtsgppss cpsgadvqgc ynkakswfhs |
| 181 | nflyigiiti cvcviqvlgm sfaltlncqi dktsqalgl |

SEQ ID NO: 173 Human FERMT3 Transcript Variant 1 cDNA Sequence
(NM_178443.2; CDS: 150-2153)

| | |
|---|---|
| 1 | ccgccctgct cgtgataagg cacaagcaag ggctgccctg aaggaagctc caaagagaaa |
| 61 | ggagggcagg aagcccacgg cccacagggg tgtagcccga gacccacctg cagcccccag |
| 121 | cccttgccag gaaagcagca gccgcagcca tggcggggat gaagacagcc tccggggact |
| 181 | acatcgactc gtcatgggag ctgcgggtgt ttgtgggaga ggaggaccca gaggccgagt |
| 241 | cggtcaccct gcgggtcact ggggagtcgc acatcggcgg ggtgctcctg aagattgtgg |
| 301 | agcagatcaa tcgcaagcag gactggtcag accatgctat ttggtgggaa cagaagaggc |
| 361 | agtggctgct gcagacccac tggacactgg acaagtacgg gatcctggcc gacgcacgcc |
| 421 | tcttctttgg gccccagcac cggcccgtca tccttcggtt gcccaaccgc cgcgcactgc |
| 481 | gcctccgtgc cagcttctcc cagcccctct tccaggctgt ggctgccatc tgccgcctcc |
| 541 | tcagcatccg gcaccccgag gagctgtccc tgctccgggc tcctgagaag aaggagaaga |
| 601 | agaagaaaga gaaggagcca gaggaagagc tctatgactt gagcaaggtt gtcttggctg |
| 661 | ggggcgtggc acctgcactg ttccggggga tgccagctca cttctcggac agcgcccaga |
| 721 | ctgaggcctg ctaccacatg ctgagccggc cccagccgcc acccgacccc ctcctgctcc |
| 781 | agcgtctgcc acggcccagc tccctgtcag acaagaccca gctccacagc aggtggctgg |
| 841 | actcgtcgcg gtgtctcatg cagcagggca tcaaggccgg ggacgcactc tggctgcgct |
| 901 | tcaagtacta cagcttcttc gatttggatc ccaagacaga ccccgtgcgg ctgacacagc |
| 961 | tgtatgagca ggcccggtgg gacctgctgc tggaggagat tgactgcacc gaggaggaga |
| 1021 | tgatggtgtt tgccgccctg cagtaccaca tcaacaagct gtcccagagc ggggaggtgg |
| 1081 | gggagccggc tggcacagac ccagggctgg acgacctgga tgtggccctg agcaacctgg |
| 1141 | aggtgaagct ggaggggtcg gcgcccacag atgtgctgga cagcctcacc accatcccag |
| 1201 | agctcaagga ccatctccga atctttcgca tcccacgaag gccccggaag ctgagcctga |
| 1261 | agggctaccg ccaacactgg gtggtgttca aggagaccac actgtcctac tacaagagcc |
| 1321 | aggacgaggc ccctggggac cccattcagc agctcaacct caagggctgt gaggtggttc |
| 1381 | ccgatgttaa cgtctccggc cagaagttct gcattaaact cctagtgccc tcccctgagg |
| 1441 | gcatgagtga gatctacctg cggtgccagg atgagcagca gtatgcccgc tggatggctg |
| 1501 | gctgccgcct ggcctccaaa ggccgcacca tggccgacag cagctacacc agcgaggtgc |
| 1561 | aggccatcct ggccttcctc agcctgcagc gcacgggcag tgggggcccg ggcaaccacc |
| 1621 | cccacggccc tgatgcctct gccgagggcc tcaaccccta cggcctcgtt gccccccgtt |
| 1681 | tccagcgaaa gttcaaggcc aagcagctca ccccacggat cctggaagcc caccagaatg |
| 1741 | tggcccagtt gtcgctggca gaggcccagc tgcgcttcat ccaggcctgg cagtccctgc |
| 1801 | ccgacttcgg catctcctat gtcatggtca ggttcaaggg cagcaggaaa gacgagatcc |
| 1861 | tgggcatcgc caacaaccga ctgatccgca tcgacttggc cgtgggcgac gtggtcaaga |
| 1921 | cctggcgttt cagcaacatg cgccagtgga atgtcaactg ggacatccgg caggtggcca |
| 1981 | tcgagtttga tgaacacatc aatgtggcct tcagctgcgt gtctgccagc tgccgaattg |
| 2041 | tacacgagta tatcgggggc tacattttcc tgtcgacgcg ggagcgggcc cgtggggagg |
| 2101 | agctggatga agacctcttc ctgcagctca ccgggggcca tgaggccttc tgagggctgt |
| 2161 | ctgattgccc ctgccctgct caccaccctg tcacagccac tcccaagccc acacccacag |
| 2221 | gggctcactg ccccacaccc gctccaggca ggcacccagc tgggcatttc acctgctgtc |
| 2281 | actgactttg tgcaggccaa ggacctggca gggccagacg ctgtaccatc acccaggcca |
| 2341 | gggatggggg tgggggtccc tgagctcatg tggtgccccc tttccttgtc tgagtggctg |
| 2401 | aggctgatac ccctgaccta tctgcagtcc cccagcacac aaggaagacc agatgtagct |
| 2461 | acaggatgat gaaacatggt ttcaaacgag ttctttcttg ttacttttta aaatttcttt |
| 2521 | tttataaatt aatattttat tgttggatcc tcaaaaaaga aaaaaaaaaa |

SEQ ID NO: 174 Human FERMT3 Isoform 1 Amino Acid Sequence (NP_848537.1)

| | |
|---|---|
| 1 | magmktasgd yidsswelrv fvgeedpeae svtlrvtges higgvllkiv eqinrkqdws |
| 61 | dhaiwweqkr qwllqthwtl dkygiladar lffgpqhrpv ilrlpnrral rlrasfsqpl |
| 121 | fqavaaicrl lsirhpeels llrapekkek kkkekepeee lydlskvvla ggvapalfrg |
| 181 | mpahfsdsaq teacyhmlsr pqpppdplll qrlprpssls dktqlhsrwl dssrclmqqg |
| 241 | ikagdalwlr fkyysffdld pktdpvrltq lyeqarwdll leeidcteee mmvfaalqyh |
| 301 | inklsqsgev gepagtdpgl ddldvalsnl evklegsapt dvldslttip elkdhlrifr |
| 361 | iprrprkltl kgyrghwvvf kettlsyyks qdeapgdpiq qlnlkgcevv pdvnvsgqkf |
| 421 | cikllvpspe gmseiylrcq deqqyarwma gcrlaskgrt madssytsev qailaflslq |
| 481 | rtgsggpgnh phgpdasaeg lnpyglvapr fqrkfkakql tprileahqn vaqlslaeaq |
| 541 | lrfiqawqsl pdfgisyvmv rfkgsrkdei lgiannrlir idlavgdvvk twrfsnmrqw |
| 601 | nvnwdirqva iefdehinva fscvsascri vheyiggyif lstrerarge eldedlflql |
| 661 | tggheaf |

SEQ ID NO: 175 Human FERMT3 Transcript Variant 2 cDNA Sequence
(NM_031471.5; CDS: 150-2141)

| | |
|---|---|
| 1 | ccgccctgct cgtgataagg cacaagcaag ggctgccctg agggaagctc caaagagaaa |
| 61 | ggagggcagg aagcccacgg cccacagggg tgtagcccga gacccacctg cagcccccag |
| 121 | cccttgccag gaaagcagca ggcggagcca tggcggggat gaagacagcc tccggggact |
| 181 | acatcgactc gtcatgggag ctgcgggtgt ttgtgggaga ggaggaccca gaggccgagt |
| 241 | cggtcaccct gcgggtcact ggggagtcgc acatcggcgg ggtgctcctg aagattgtgg |
| 301 | agcagatcaa tcgcaagcag gactggtcag accatgctat ttggtgggaa cagaagaggc |
| 361 | agtggctgct gcagacccac tggacactgg acaagtacgg gatcctggcc gacgcacgcc |
| 421 | tcttctttgg gccccagcac cggcccgtca tccttcggtt gcccaaccgc cgcgcactgc |
| 481 | gcctccgtgc cagcttctcc cagcccctct tccaggctgt ggctgccatc tgccgcctcc |
| 541 | tcagcatccg gcaccccgag gagctgtccc tgctccgggc tcctgagaag aaggagaaga |
| 601 | agaagaaaga gaaggagcca gaggaagagc tctatgactt gagcaaggtt gtcttggctg |
| 661 | ggggcgtggc acctgcactg ttccggggga tgccagctca cttctcggac agcgcccaga |
| 721 | ctgaggcctg ctaccacatg ctgagccggc cccagccgcc acccgacccc ctcctgctcc |
| 781 | agcgtctgcc acggcccagc tccctgtcag acaagaccca gctccacagc aggtggctgg |
| 841 | actcgtcgcg gtgtctcatg cagcagggca tcaaggccgg ggacgcactc tggctgcgct |
| 901 | tcaagtacta cagcttcttc gatttggatc ccaagacaga ccccgtgcgg ctgacacagc |
| 961 | tgtatgagca ggcccggtgg gacctgctgc tggaggagat tgactgcacc gaggaggaga |
| 1021 | tgatggtgtt tgccgccctg cagtaccaca tcaacaagct gtcccagagc ggggaggtgg |
| 1081 | gggagccggc tggcacagac ccagggctgg acgacctgga tgtggccctg agcaacctgg |
| 1141 | aggtgaagct ggaggggtcg gcgcccacag atgtgctgga cagcctcacc accatcccag |
| 1201 | agctcaagga ccatctccga atctttcggc cccggaagct gaccctgaag ggctaccgcc |
| 1261 | aacactgggt ggtgttcaag gagaccacac tgtcctacta caagagccag gacgaggccc |
| 1321 | ctggggaccc cattcagcag ctcaacctca agggctgtga ggtggttccc gatgttaacg |
| 1381 | tctccggcca gaagttctgc attaaactcc tagtgccctc ccctgagggc atgagtgaga |
| 1441 | tctacctgcg gtgccaggat gagcagcagt atgcccgctg gatggctggc tgccgcctgg |
| 1501 | cctccaaagg ccgcaccatg gccgacagca gctacaccag cgaggtgcag gccatcctgg |
| 1561 | ccttcctcag cctgcagcgc acgggcagtg ggggcccggg caaccacccc cacggccctg |
| 1621 | atgcctctgc cgagggcctc aacccctacg gcctcgttgc cccccgtttc cagcgaaagt |
| 1681 | tcaaggccaa gcagctcacc ccacggatcc tggaagccca ccagaatgtg gcccagttgt |
| 1741 | cgctggcaga ggcccagctg cgcttcatcc aggcctggca gtccctgccc gacttcggca |
| 1801 | tctcctatgt catggtcagg ttcaagggca gcaggaaaga cgagatcctg ggcatcgcca |
| 1861 | acaaccgact gatccgcatc gacttggccg tgggcgacgt ggtcaagacc tggcgtttca |
| 1921 | gcaacatgcg ccagtggaat gtcaactggg acatccggca ggtggccatc gagtttgatg |
| 1981 | aacacatcaa tgtggccttc agctgcgtgt ctgccagctg ccgaattgta cacgagtata |
| 2041 | tcgggggcta cattttcctg tcgacgcggg agcgggcccg tggggaggag ctggatgaag |
| 2101 | acctcttcct gcagctcacc gggggccatg aggccttctg agggctgtct gattgcccct |
| 2161 | gccctgctca ccaccctgtc acagccactc ccaagcccac acccacaggg gctcactgcc |
| 2221 | ccacacccgc tccaggcagg cacccagctg ggcatttcac ctgctgtcac tgactttgtg |
| 2281 | caggccaagg acctggcagg gccagacgct gtaccatcac ccaggccagg gatgggggtg |
| 2341 | ggggtccctg agctcatgtg gtgccccctt tccttgtctg agtggctgag gctgataccc |
| 2401 | ctgacctatc tgcagtcccc cagcacacaa ggaagaccag atgtagctac aggatgatga |
| 2461 | aacatggttt caaacgagtt ctttcttgtt actttttaaa atttcttttt tataaattaa |
| 2521 | tattttattg ttggatcctc aaaaaaaaaa aaaaaaaa |

SEQ ID NO: 176 Human FERMT3 Isoform 2 Amino Acid Sequence (NP_113659.3)

| | |
|---|---|
| 1 | magmktasgd yidsswelrv fvgeedpeae svtlrvtges higgvllkiv eqinrkqdws |
| 61 | dhaiwweqkr qwllqthwtl dkygiladar lffgpghrpv ilrlpnrral rlrasfsqpl |
| 121 | fqavaaicrl lsirhpeels llrapekkek kkkekepeee lydlskvvla ggvapalfrg |
| 181 | mpahfsdsaq teacyhmlsr pqpppdplll qrlprpssls dktqlhsrwl dssrclmqqg |
| 241 | ikagdalwlr fkyysffdld pktdpvrltq lyeqarwdll leeidcteee mmvfaalqyh |
| 301 | inklsqsgev gepagtdpgl ddldvalsnl evklegsapt dvldslttip elkdhlrifr |
| 361 | prkltlkgyr qhwvvfkett lsyyksqdea pgdpiqqlnl kgcevvpdvn vsgqkfcikl |
| 421 | lvpspegmse iylrcqdeqq yarwmagcrl askgrtmads sytsevqqil aflslqrtgs |
| 481 | ggpgnhphgp dasaeglnpy glvaprfqrk fkakqltpri leahqnvaql slaeaqlrfi |
| 541 | qawqslpdfg isyvmvrfkg srkdeilgia nnrliridla vgdvvktwrf snmrqwnvnw |
| 601 | dirqvaiefd ehinvafscv sascrivhey iggyiflstr erargeelde dlflqltggh |
| 661 | eaf |

SEQ ID NO: 177 Mouse FERMT3 Transcript Variant 2 cDNA Sequence
(NM_001362399.1; CDS: 210-2207)

| | |
|---|---|
| 1 | gtggagccct ctgcgtgctg gcagtgtgct tcctgtaccc agcaacagcc cgccctgggg |
| 61 | agggactagt acaactttca tgataaggcc actacaggct gacctctgta ggaagcaact |
| 121 | gagcaggacc aggccagacc agggtaacag gtgtcccact gccacctgca gctgcagccc |
| 181 | gttgcaggct agcagaaaca gccgcagcca tggcgggtat gaagacagcc tccggggact |
| 241 | atatcgactc ttcctgggag ctgagggtgt ttgtgggcga ggaggaccct gaggcccagt |
| 301 | ctgtcacact ccgagtcacg ggggagtcgc acattggtgg ggtacttctg aagatcgtgg |
| 361 | aggaaatcaa tcgcaagcag gactggtctg accatgccat ttggtgggag cagaagagac |
| 421 | agtggctgct gcagacacac tggacgctgg acaagtacgg gattctggcc gatgcccgcc |
| 481 | tcttctttgg gccacagcac cgacctgtca tcctgcgact gcccaacaga cgtgtgctgc |
| 541 | ggcttcgggc cagtttctcc aagcccctct tccaaacggt ggctgccatc tgccgcctcc |
| 601 | tcagtatcag gcacccagag gagctgtctc tgctgcgtgc tccagagaag aaggagaaga |
| 661 | agaagaagga gaaggagcct gaagaggagg tgcatgacct gacaaaggtt gtcttagctg |
| 721 | gcggtgtggc acccacttta ttcagaggca tgccagcaca cttctcggac agcgcccaga |
| 781 | ctgaggcttg ttaccacatg ctgagccggc cacagccggc acctgaccct ctcctgctcc |
| 841 | agcgcctgcc aaggcccagc tccctgcctg acaagaccca gctccacagc aggtggctgg |
| 901 | attcatctcg gtgcctcatg cagcaaggta tcaaggccgg ggatgtgctt tggctgcgat |
| 961 | ttaagtacta cagctttttt gacctggatc ccaagacaga ccctgtgcgg ctgacccagc |
| 1021 | tgtatgagca ggcccgctgg gacttactga cagaggagat cgactgcact gaagaagaga |
| 1081 | tgatggtgtt tgctgccctg cagtaccaca tcaacaaact gaccctgagc ggggatgtgg |
| 1141 | gtgagctggc ctctggggac ctgggactgg atgacctgga tgcagccctg aacaacctgg |
| 1201 | aggtgaagct gaaggggtca gcaccctcag acatgctgga tagtctcact accatcccag |
| 1261 | aactcaagga ccatctccgg atcttccggc cccggaagct gaccctgaag ggctaccgcc |
| 1321 | agtactgggt ggtatttaag gacaccacac tgtcctacta taagagccaa gatgaagcac |
| 1381 | cgggggaccc tacccagcag ctcaacctca agggctgtga ggtggtccct gatgtcaatg |
| 1441 | tctctggcca gaagttctgc atcaaactcc tggtaccctc accagagggc atgagtgaga |
| 1501 | tctacctgag gtgccaggat gaacagcagt acgcacagtg gatggctgcc tgccgactgg |
| 1561 | cctccaaggg ccgcaccatg gcggacagca gctatgccag cgaggtgcag gccattctgg |
| 1621 | ccttcctcag cctgcagcgg gcaggtggta gcaatggagg ctcagggaac aaacctcaag |
| 1681 | gtcctgaagc ccctgctgag ggccttaacc cctatggcct cgtggctcct cggttccagc |
| 1741 | gaaagttcaa ggccaagcag ctcaccccaa ggatcctgga agctcaccaa aacgtggccc |
| 1801 | aactctcact gaccgaggcc cagctgcgct ttatccaggc ctggcaatcc ttgcccgact |
| 1861 | ttggcatctc ctatgtgatg gtcaggttca agggcagcag gaaagatgag atcctaggca |
| 1921 | ttgccaacaa ccgactcatc cggattgatc tggccgtggg tgacgtggtc aagacctggc |
| 1981 | gcttcagcaa catgcggcaa tggaacgtca actgggacat acggcaggtg gccattgagt |
| 2041 | tcgatgaaca catcaatgtg gccttcagct gtgtgtctgc tagctgccgc atcgtgcacg |
| 2101 | agtacatcgg gggttacatc ttcctgtcca cccgagagcg ggcccgaggg gaagagctgg |
| 2161 | atgaggatct gttcctgcag cttacaggag gccatgaggc cttctgaggg cagcccctgc |
| 2221 | tccactcacc acctccaggg accccccaaa ggccacaccc acctgaacac actgctccat |
| 2281 | cactcccggg atgtacactg ctgggcaact tcacttgttg tcaccaggca ggccagggcc |
| 2341 | ctcgctgggg taggtgctac caccagccag cctagggatg atgggccctg agccacacag |
| 2401 | agccacctcc ctcaccctgt tggcagaagc tcacaccccc gacctctatg tagtccctaa |
| 2461 | gcacacgacg aatgccagac acggatgagg aaccacaaat cctggtttga agctggactt |
| 2521 | tctttctgtg tctgtttcag gttttttaat aaataaatat tttattgttg gatctttctt |
| 2581 | cttcctcctc |

SEQ ID NO: 178 Mouse FERMT3 Transcript Variant 1 cDNA Sequence
(NM_153795.2; CDS: 207-2204)

| | |
|---|---|
| 1 | gtggagccct ctgcgtgctg gcagtgtgct tcctgtaccc agcaacagcc cgccctgggg |
| 61 | agggactagt acaactttca tgataaggcc actacaggct gacctctgaa ggaagcaact |
| 121 | gagcaggacc aggccagacc agggtaacag gtgtcccact gccacctgca gctgcagccc |
| 181 | gttgcaggct agaaacagcc gcagccatgg cgggtatgaa gacagcctcc ggggactata |
| 241 | tcgactcttc ctgggagctg agggtgtttg tgggcgagga ggaccctgag gcccagtctg |
| 301 | tcacactccg agtcacgggg gagtcgcaca ttggtggggt acttctgaag atcgtggagg |
| 361 | aaatcaatcg caagcaggac tggtctgacc atgccatttg gtgggagcag aaaagacagt |
| 421 | ggctgctgca gacacactgg acgctggaca agtacgggat tctggccgat gcccgcctct |
| 481 | tctttgggcc acagcaccga cctgtcatcc tgcgactgcc caacagacgt gtgctgcggc |
| 541 | ttcgggccag tttctccaag cccctcttcc aaacggtggc tgccatctgc cgcctcctca |
| 601 | gtatcaggca cccagaggag ctgtctctgc tgcgtgctcc agagaagaag gagaagaaga |
| 661 | agaaggagaa ggagcctgaa gaggaggtgc atgacctgac aaaggttgtc ttagctggcg |
| 721 | gtgtggcacc cactttattc agaggcatgc cagcacactt ctcggacagc gcccagactg |
| 781 | aggcttgtta ccacatgctg agccggccac agccggcacc tgaccctctc ctgctccagc |
| 841 | gcctgccaag gcccagctcc ctgcctgaca agacccagct ccacagcagg tggctggatt |
| 901 | catctcggtg cctcatgcag caaggtatca aggccgggga tgtgctttgg ctgcgattta |
| 961 | agtactacag cttttttgac ctggatccca agacagaccc tgtgcggctg acccagctgt |
| 1021 | atgagcaggc ccgctgggac ttactgacag aggagatcga ctgcactgaa gaagagatga |
| 1081 | tggtgtttgc tgccctgcag taccacatca acaaactgac cctgagcggg gatgtgggtg |
| 1141 | agctggcctc tggggacctg ggactggatg acctggatgc agccctgaac aacctggagg |
| 1201 | tgaagctgaa ggggtcagca ccctcagaca tgctggatag tctcactacc atcccagaac |
| 1261 | tcaaggacca tctccggatc ttccggcccc ggaagctgac cctgaagggc taccgccagt |
| 1321 | actgggtggt atttaaggac accacactgt cctactataa gagccaagat gaagcaccgg |
| 1381 | gggaccctac ccagcagctc aacctcaagg gctgtgaggt ggtccctgat gtcaatgtct |
| 1441 | ctggccagaa gttctgcatc aaactcctgg taccctcacc agagggcatg agtgagatct |
| 1501 | acctgaggtg ccaggatgaa cagcagtacg cacagtggat ggctgcctgc cgactggcct |
| 1561 | ccaagggccg caccatggcg gacagcagct atgccagcga ggtgcaggcc attctggcct |
| 1621 | tcctcagcct gcagcgggca ggtggtagca atggaggctc agggaacaaa cctcaaggtc |
| 1681 | ctgaagcccc tgctgagggc cttaacccct atggcctcgt ggctcctcgg ttccagcgaa |
| 1741 | agttcaaggc caagcagctc accccaagga tcctggaagc tcaccaaaac gtggcccaac |
| 1801 | tctcactgac cgaggcccag ctgcgcttta tccaggcctg gcaatccttg cccgactttg |
| 1861 | gcatctccta tgtgatggtc aggttcaagg gcagcaggaa agatgagatc ctaggcattg |
| 1921 | ccaacaaccg actcatccgg attgatctgg ccgtgggtga cgtggtcaag acctggcgct |
| 1981 | tcagcaacat gcggcaatgg aacgtcaact gggacatacg gcaggtggcc attgagttcg |
| 2041 | atgaacacat caatgtggcc ttcagctgtg tgtctgctag ctgccgcatc gtgcacgagt |
| 2101 | acatcggggg ttacatcttc ctgtccaccc gagagcgggc ccgaggggaa gagctggatg |
| 2161 | aggatctgtt cctgcagctt acaggaggcc atgaggcctt ctgagggcag cccctgctcc |
| 2221 | actcaccacc tccagggacc ccccaaaggc cacacccacc tgaacacact gctccatcac |
| 2281 | tcccgggatg tacactgctg ggcaacttca cttgttgtca ccaggcaggc cagggccctc |
| 2341 | gctggggtag gtgctaccac cagccagcct agggatgatg ggccctgagc cacacagagc |
| 2401 | ccctcctctc accctgttgg cagaagctca cacccccgac ctctatgtag tccctaagca |
| 2461 | cacgacccat gccagacacg gatgaggaac cacaaatcct ggtttgaagc tggactttct |
| 2521 | ttctgtgtct gtttcaggtt ttttaataaa taaatatttt attgttggat ctttcttctt |
| 2581 | cctcctc |

SEQ ID NO: 179 Mouse FERMT3 Amino Acid Sequence (NP_001349328.1 and
NP_722490.1)

| | |
|---|---|
| 1 | magmktasgd yidsswelrv fvgeedpeaq svtlrvtges higgvllkiv eeinrkqdws |
| 61 | dhaiwweqkr qwllqthwtl dkygiladar lffgpqhrpv ilrlpnrrvl rlrasfskpl |
| 121 | fqtvaaicrl lsirhpeels llrapekkek kkkekepeee vhdltkvvla ggvaptlfrg |
| 181 | mpahfsdsaq teacyhmlsr pqpapdplll qrlprpsslp dktqlhsrwl dssrclmqqg |
| 241 | ikagdvlwlr fkyysffdld pktdpvrltq lyeqarwdll teeidcteee mmvfaalqyh |
| 301 | inkltlsgdv gelasgdlgi ddldaalnnl evklkgsaps dmldslttip elkdhlrifr |
| 361 | prkltlkgyr qywvvfkdtt lsyyksqdea pgdptqqlnl kgcevvpdvn vsgqkfcikl |
| 421 | lvpspegmse iylrcqdeqq yaqwmaacrl askgrtmads syasevqail aflslqragg |
| 481 | snggsgnkpq gpeapaegln pyglvaprfq rkfkakqltp rileahqnva qlslteaqlr |
| 541 | fiqawqslpd fgisyvmvrf kgsrkdeilg iannrlirid lavgdvvktw rfsnmrqwnv |
| 601 | nwdirqvaie fdehinvafs cvsascrivh eyiggyifls trerargeel dedlflqltg |
| 661 | gheaf |

SEQ ID NO: 180 Human CD37 Transcript Variant 1 cDNA Sequence (NM_001774.2;
CDS: 122-967)

| | |
|---|---|
| 1 | ttcctttctc tctcagctct ccgtctctct ttctctctca gcctctttct ttctccctgt |
| 61 | ctcccccact gtcagcacct cttctgtgtg gtgagtggac cgcttacccc actaggtgaa |
| 121 | gatgtcagcc caggagagct gcctcagcct catcaagtac ttcctcttcg ttttcaacct |
| 181 | cttcttcttc gtcctcggca gcctgatctt ctgcttcggc atctggatcc tcattgacaa |
| 241 | gaccagcttc gtgtcctttg tgggcttggc cttcgtgcct ctgcagatct ggtccaaagt |
| 301 | cctggccatc tcaggaatct tcaccatggg catcgccctc ctgggttgtg tgggggccct |
| 361 | caaggagctc cgctgcctcc tgggcctgta ttttgggatg ctgctgctcc tgtttgccac |
| 421 | acagatcacc ctgggaatcc tcatctccac tcagcgggcc cagctggagc gaagcttgcg |
| 481 | ggacgtcgta gagaaaacca tccaaaagta cggcaccaac cccgaggaga ccgcggccga |
| 541 | ggagagctgg gactatgtgc agttccagct gcgctgctgc ggctggcact acccgcagga |
| 601 | ctggttccaa gtcctcatcc tgagaggtaa cgggtcggag gcgcaccgcg tgccctgctc |
| 661 | ctgctacaac ttgtcggcga ccaacgactc cacaatccta gataaggtga tcttgcccca |
| 721 | gctcagcagg cttggacacc tggcgcggtc cagacacagt gcagacatct gcgctgtccc |
| 781 | tgcagagagc cacatctacc gcgagggctg cgcgcagggc ctccagaagt ggctgcacaa |
| 841 | caaccttatt tccatagtgg gcatttgcct gggcgtcggc ctactcgagc tcgggttcat |
| 901 | gacgctctcg atattcctgt gcagaaacct ggaccacgtc tacaaccggc tcgctcgata |
| 961 | ccgttaggcc ccgccctccc caaagtcccg ccccgccccc gtcacgtgcg ctgggcactt |
| 1021 | ccctgctgcc tgtaaatatt tgtttaatcc ccagttcgcc tggagccctc cgccttcaca |
| 1081 | ttcccctggg gacccacgtg gctgcgtgcc cctgctgctg tcacctctcc cacgggacct |
| 1141 | ggggctttcg tccacagctt cctgtcccca tctgtcggcc taccaccacc cacaagatta |
| 1201 | tttttcaccc aaacctcaaa taaatcccct gcgtttttgg taaaaaaaaa aaaaaaaaaa |
| 1261 | aaa |

SEQ ID NO: 181 Human CD37 Isoform A Amino Acid Sequence (NP_001765.1)

| | |
|---|---|
| 1 | msaqesclsl ikyflfvfnl fffvlgslif cfgiwilidk tsfvsfvgla fvplqiwskv |
| 61 | laisgiftmg iallgcvgal kelrcllgly fgmllllfat qitlgilist qraqlerslr |
| 121 | dvvektiqky gtnpeetaae eswdyvqfqa rccgwhypqd wfqvlilrgn gseahrvpcs |
| 181 | cynlsatnds tildkvilpq lsrlghlars rhsadicavp aeshiyregc aqglqkwlhn |
| 241 | nlisivgicl gvgllelgfm tlsiflcrnl dhvynrlary r |

SEQ ID NO: 182 Human CD37 Transcript Variant 2 cDNA Sequence
(NM_001040031.1; CDS: 292-933)

| | |
|---|---|
| 1 | ttcctttctc tctcagctct ccgtctctct ttctctctca gcctctttct ttctccctgt |
| 61 | ctcccccact gtcagcacct cttctgtgtg gtgagtggac cgcttacccc actaggtgaa |
| 121 | gatgtcagcc caggagagct gcctcagcct catcaagtcc tcggcagcct gatcttctgc |
| 181 | ttcggcatct ggatcctcat tgacaagacc agcttcgtgt cctttgtggg cttggccttc |
| 241 | gtgcctgtgc agatctggtc caaagtcctg gccatctcag gaatcttcac catgggcatc |
| 301 | gccctcctgg gttgtgtggg ggccctcaag gagctccgct gcctcctggg cctgtatttt |
| 361 | gggatgctgc tgctcctgtt tgccacacag atcaccctgg gaatcctcat ctccactcag |
| 421 | cgggcccagc tggagcgaag cttgcgggac gtcgtagaga aaaccatcca aaagtacggc |
| 481 | accaaccccg aggagaccgc ggccgaggag agctgggact atgtgcagtt ccagctgcgc |
| 541 | tgctgcggct ggcactaccc gcaggactgg ttccaagtcc tcatcctgag aggtaacggg |
| 601 | tcggaggcgc accgcgtgcc ctgctcctgc tacaacttgt cggcgaccaa cgactccaca |
| 661 | atcctagsta aggtgatctt gccccagctc agcaggcttg gacacctggc gcggtccaga |
| 721 | cacagtgcag acatctgcgc tgtccctgca gagagccaca tctaccgcga gggctgcgcg |
| 781 | cagggcctcc agaagtggct gcacaacaac cttatttcca tagtgggcat ttgcctgggc |
| 841 | gtcggcctac tcgagctcgg gttcatgacg ctctcgatat tcctgtgcag aaacctggac |
| 901 | cacgtctaca accggctcgc tcgataccgt taggccccgc cctccccaaa gtcccgcccc |
| 961 | gcccccgtca cgtgcgctgg gcacttccct gctgcctgta aatatttgtt taatccccag |
| 1021 | ttcgcctgga gccctccgcc ttcacattcc cctggggacc cacgtggctg cgtgccacctg |
| 1081 | ctgctgtcac ctctcccacg ggacctgggg ctttcgtcca cagcttcctg tccccatctg |
| 1141 | tcggcctacc accacccaca agattatttt tcacccaaac ctcaaataaa tcccctgcgt |
| 1201 | ttttggtaaa aaaaaaaaaa aaaaaaaaa |

SEQ ID NO: 183 Human CD37 Isoform B Amino Acid Sequence (NP_001035120.1)

| | |
|---|---|
| 1 | mgiallgcvg alkelrcllg lyfgmllllf atqitlgili stqraqlers lrdvvektiq |
| 61 | kygtnpeeta aeeswdyvqf qlrccgwhyp qdwfqvlilr gngseahrvp cscynlsatn |
| 121 | dstildkvil pqlsrlghla rsrhsadica vpaeshiyre gcaqglqkwl hnnlisivdi |
| 181 | clgvgllelg fmtlsiflcr nldhyynrla ryr |

SEQ ID NO: 184 Mouse CD37 Transcript Variant 1 cDNA Sequence
(NM_001290802.1; CDS: 97-1008)

| | |
|---|---|
| 1 | tgtctgtgag gttgaactct gcagaaacaa ccttgaagtc ccaggaccca cgtgagtgga |
| 61 | agtccatcag aagcccctgt gcgccctgcc tgagctatgg acacctgcga ggaacccatt |
| 121 | gtgtccctgg cacccacata ttccaaggac cctcaggcga agatgtccgc ccaagagagt |
| 181 | tgcctcagcc tcatcaagta cttcctcttc gttttcaacc tcttcttctt tgtactaggc |
| 241 | ggcctgattt tctgcttcgg cacctggatc ctcattgaca agaccagctt cgtgtccttt |
| 301 | gtgggtttgt ccttcgtgcc actgcagact tggtccaagg tcctggctgt ctcaggtgtc |
| 361 | ctcaccatgg ccctggctct cctgggctgt gtgggggctc taaaggagct gcgctgtctc |
| 421 | ctgggcctgt attttggaat gctgctgctc ctgtttgcca cacagattac cctgggcatc |
| 481 | ctcatttcca ctcagcgggt ccggctggag cgaagggtgc aggaattggt gttgaggacg |
| 541 | atccagagct accgcacgaa tccggctgag acagcagccg aggagagttg ggactatgca |
| 601 | cagttccagc tgcgctgctg cggctggcaa tctccgcggt actggaacaa ggcccagatg |
| 661 | ctgaaagcga acgagtctga ggagcccttt gtgccctgct cctgctacaa ctccacggcg |
| 721 | accaatgact ccaccgtctt tgataagctc tttttctccc agctaagccg gttggggccg |
| 781 | cgggcgaagc tgaggcagac tgctgacata tgtgcactcc ctgcaaaagc tcacatctac |
| 841 | cgtgagggct gcgcgcagag cctccagaag tggctgcaca acaatatcat ctccatagtg |
| 901 | ggaatctgtc tgggagtcgg tcttcttgag ctcggcttca tgacgctctc aatattcctg |
| 961 | tgtagaaatc tggatcacgt ctatgaccgg ctggcccggt accgctaggc cccagcccac |
| 1021 | ccatcggcac ccacgcacct ccatgcagtc aggaaatgtt tccttcctgg ttggtcacag |
| 1081 | gcttggagcc catgcctcag gcctcatccc ctggggaccc agctgtctgc cctatcgaca |
| 1141 | gccttcactt tccccacatg gccagggact tttgtgccca gcttccttcc cttcctggct |
| 1201 | ccctcctccc cacaccatct gtctgccacc tcccacatga cacctcaaat aaatcccctt |
| 1261 | tgggtttttg gcttttt |

SEQ ID NO: 185 Mouse CD37 Isoform 1 Amino Acid Sequence (NP_001277731.1)

| | |
|---|---|
| 1 | mdtceepivs laptvskdpq akmsaqescl slikyflfvf nlfffvlggl ifcfgtwili |
| 61 | dktsfvsfvg isfvplqtws kvlavsgvlt malallgcvg alkelrcllg lyfgmllllf |
| 121 | atqitlgili stqrvrlerr vqelvlrtiq syrtnpdeta aeeswdyaqf qlrccgwqsp |
| 181 | rdwnkaqmlk aneseepfvp cscynstatn dstvfdklff sqlsrlgpra klrqtadica |
| 141 | lpakahiyre gcaqslqkwl hnniisivgi clgvgllelg fmtlsiflcr nldhvydrla |
| 301 | ryr |

SEQ ID NO: 186 Mouse CD37 Transcript Variant 2 cDNA Sequence
(NM_001290804.1; CDS: 97-933)

| | |
|---|---|
| 1 | tgtctgtgag gttgaactct gcagaaacaa ccttgaagtc ccaggaccca cgtgagtgga |
| 61 | agtccatcag aagcccctgt gcgccctgcc tgagctatgg acacctgcga ggaacccatt |
| 121 | gtgtccctgg cacccacata ttccaaggac cctcaggtac taggcggcct gattttctgc |
| 181 | ttcggcacct ggatcctcat tgacaagacc agcttcgtgt cctttgtggg tttgtccttc |
| 241 | gtgccactgc agacttggtc caaggtcctg gctgtctcag gtgtcctcac catggccctg |
| 301 | gctctcctgg gctgtgtggg ggctctaaag gagctgcgct gtctcctggg cctgtatttt |
| 361 | ggaatgctgc tgctcctgtt tgccacacag attaccctgg gcatcctcat ttccactcag |
| 421 | cgggtccggc tggagcgaag ggtgcaggaa ttggtgttga ggacgatcca gagctaccgc |
| 481 | acgaatccgg atgagacagc agccgaggag agttgggact atgcacagtt ccagctgcgc |
| 541 | tgctgcggct ggcaatctcc gcgggactgg aacaaggccc agatgctgaa agcgaacgag |
| 601 | tctgaggagc cctttgtgcc ctgctcctgc tacaactcca cggcgaccaa tgactccacc |
| 661 | gtctttgata agctcttttt ctcccagcta agccggttgg ggccgcgggc gaagctgagg |
| 721 | cagactgctg acatatgtgc actccctgca aaagctcaca tctaccgtga gggctgcgcg |
| 781 | cagagcctcc agaagtggct gcacaacaat atcatctcca tagtgggaat ctgtctggga |
| 841 | gtcggtcttc ttgagctcgg cttcatgacg ctctcaatat tcctgtgtag aaatctggat |
| 901 | cacgtctatg accggctggc ccggtaccgc taggccccag cccacccatc ggcacccacg |
| 961 | cacctccatg cagtcaggaa atgtttcctt cctggttggt cacaggcttg gagcccatgc |
| 1021 | ctcaggcctc atcccctggg gacccagctg tctgccctat cgacagcctt cactttcccc |
| 1081 | acatggccag ggacttttgt gcccagcttc cttcccttcc tggctccctc ctccccacac |
| 1141 | catctgtctg ccacctccca catgacacct caaataaatc ccctttgggt ttttggcttt |
| 1201 | tt |

SEQ ID NO: 187 Mouse CD37 Isoform 2 Amino Acid Sequence (NP_001277733.1)

| | |
|---|---|
| 1 | mdtceepivs laptyskdpq vlgglifcfg twilidktsf vsfvglsfvp lqtwskvlav |
| 61 | sgvltmalal lgcvgalkel rcllglyfgm llllfatqit lgilistqrv rlerrvqelv |
| 121 | lrtiqsyrtn pdetaaeesw dyaqfqlrcc gwgsprdwnk aqmlkanese epfvpcscyn |
| 181 | statndstvf dklffsqlsr lgpraklrqt adicalpaka hiyregcaqs lqkwlhnnii |
| 241 | sivgiclgvg llelgfmtls iflcrnldhv ydrlaryr |

SEQ ID NO: 188 Mouse CD37 Transcript Variant 3 cDNA Sequence (NM_007645.4;
CDS: 12-957)

| | |
|---|---|
| 1 | tttgtgcctc tcagtctctg tctttctttg cccactattc cccttccccc ctgcggccaa |
| 61 | cacctcttct gtctgggttt ctcgagtggg tcattcccca tctaggcgaa gatgtccgcc |
| 121 | caagagagtt gcctcagcct catcaagtac ttcctcttcg ttttcaacct cttcttcttt |
| 181 | gtactaggcg gcctgatttt ctgcttcggc acctggatcc tcattgacaa gaccagcttc |
| 241 | gtgtcctttg tgggtttgtc cttcgtgcca ctgcagactt ggtccaaggt cctggctgtc |
| 301 | tcaggtgtcc tcaccatggc cctggctctc ctgggctgtg tgggggctct aaaggagctg |
| 361 | cgctgtctcc tgggcctgta ttttggaatg ctgctgctcc tgtttgccac acagattacc |
| 421 | ctgggcatcc tcatttccac tcagcgggtc cggctggagc gaagggtgca ggaattggtg |
| 481 | ttgaggacga tccagagcta ccgcacgaat ccggatgaga cagcagccga ggagagttgg |
| 541 | gactatgcac agttccagct gcgctgctgc ggctggcaat ctccgcggga ctggaacaag |
| 601 | gcccagatgc tgaaagcgaa cgagtctgag gagccctttg tgccctgctc ctgctacaac |
| 661 | tccacggcga ccaatgactc caccgtcttt gataagctct ttttctccca gctaagccgg |
| 721 | ttggggccgc gggcgaagct gaggcagact gctgacatat gtgcactccc tgcaaaagct |
| 781 | cacatctacc gtgagggctg cgcgcagagc ctccagaagt ggctgcacaa caatatcatc |
| 841 | tccatagtgg gaatctgtct gggagtcggt cttcttgagc tcggcttcat gacgctctca |
| 901 | atattcctgt gtagaaatct ggatcacgtc tatgaccggc tggcccggta ccgctaggcc |
| 961 | ccagcccacc catcggcacc cacgcacctc catgcagtca ggaaatgttt ccttcctggt |
| 1021 | tggtcacagg cttggagccc atgcctcagg cctcatcccc tggggaccca gctgtctgcc |
| 1081 | ctatcgacag ccttcacttt ccccacatgg ccagggactt ttgtgcccag cttccttccc |
| 1141 | ttcctggctc cctcctcccc acaccatctg tctgccacct cccacatgac acctcaaata |
| 1201 | aatccccttt gggtttttgg cttttt |

SEQ ID NO: 189 Mouse CD37 Isoform 3 Amino Acid Sequence (NP_031671.1)

| | |
|---|---|
| 1 | msaqesclsl ikyflfvfnl fffvlgglif cfgtwilidk tsfvsfvgls fvplqtwskv |
| 61 | lavsgvltma lallgcvgal kelrcllgly fgmllllfat qitlgilist qrvrlerrvq |
| 121 | elvlrtiqsy rtnpdetaae eswdyaqfql rccgwqsprd wnkaqmlkan eseepfvpcs |
| 181 | cynstatnds tvfdklffsq lsrlgprakl rqtadicalp akahiyregc aqslqkwlhn |
| 241 | niisivgicl gvgllelgfm tlsiflcrnl dhvydrlary r |

SEQ ID NO: 190 Human CXorf21 cDNA Sequence (NM_025159.2; CDS: 396-1301)

| | |
|---|---|
| 1 | attctgtggc ttgatgtggc tttgtcagtt actgcctccc cacctcccgg gacttcccac |
| 61 | tgcctcttta gcacaaatcg gacttgaaca aaaacaactc tttttccatg ggaccaatta |
| 121 | taaaaagaat ggctttgcac tgagcagctc agtctactgg tctcatcagt gaaaggcttg |
| 181 | tgaaaatttc tggaaagagc attggctggc ttgttcatct ctctgtttgg tcagttgctg |
| 241 | tgtttctgcc ggatcagatg agcagattgg ttagtgaagg tcagtgtgag aagaagattg |
| 301 | tttctgagct tgagaagctt ctggaggatt gaagagtatt tgaagtctgt gtcaaacatc |
| 361 | catatcataa gtggaatttt ggagatattc aaagaatgct gtcagaaggg tatctcagtg |
| 421 | gacttgagta ctggaatgac atccactgga gttgtgcctc ttataatgag caggtggctg |
| 481 | gggaaaagga agaggagaca aattctgttg ctaccctttc ctattcctct gtggatgaaa |
| 541 | cacaagtcag aagtctctac gtgagctgca aatcatctgg caagtttatc tcttcagtgc |
| 601 | attcaagaga gagccaacat agcagaagtc agagagtcac agtgctgcag acaaacccca |
| 661 | atcctgtgtt tgaaagccca aacttggctg cagttgaaat atgtagagat gccagcagag |
| 721 | agacctactt ggttccatct tcttgcaaaa gtatttgcaa gaattataat gacttacaga |
| 781 | ttgcaggggg ccaggtgatg gccattaatt cagtgacaac agattttccc tctgagagca |
| 841 | gttttgaata tggccctttg ctgaagtcat ctgagattcc tttacccatg gaggattcca |
| 901 | tttctactca gcccagtgac tttccccaaa aacctatcca gcggtactca tcctattgga |
| 961 | gaataacaag catcaaagag aaaagcagct tgcaaatgca gaatcctatt tctaatgcag |
| 1021 | ttctgaatga gtacctggag cagaaggttg tggagttata taaacagtac attatggaca |
| 1081 | ccgtgtttca tgacagttct cctacccaga ttctggcgtc tgaactcatc atgacaagtg |
| 1141 | tagaccaaat cagtcttcaa gtgtctagag agaagaatct ggagacctca aaagccagag |
| 1201 | atatagtctt tagccgccta ttgcaattga tgtcaactga aattactgaa attagcactc |
| 1261 | ctagtctcca tatttctcag tatagcaatg taaatccata gagaggatgc ttccattact |
| 1321 | gtctcgcatt tacttaaaca tgaagcaaca ctttatccat ttattctgag aatgtgcagg |
| 1381 | aggggttagt gaaggggaat taagggctag agaagtaaag atcagctgga agtcatgtgt |
| 1441 | gaatcatgga gaaatctcat aatattacac ttgatgaagg aatatggtaa gaggagccat |
| 1501 | aaggaatgat ttcaaagaga ggtgtgtaca gcaaggaagc aaaaataaaa atgatcaaaa |
| 1561 | aagaaacggt taattcattt gaagaaacat aggaattaaa aaagaagaca gtcaaatgag |
| 1621 | atgaaacaaa aaggccaaaa ggtggccggg tgtagtggct cacgcctgta atcccagcat |
| 1681 | ttcggaggct gatgtgggtg gattacctga cgtcaggaat tcgagaccgg cctggccaat |
| 1741 | atggtgaaac cccatctcta ctaaaaatac aaaaaattag ctggacatgg tggtgggtac |
| 1801 | ctgtaatccc agctactagg gaggctgagg caggagaatc tcttgaacct gagaggcaga |
| 1861 | ggttgcagtg agccgaggtc gctccattgc actccagcct gggcaacaag agtgagactc |
| 1921 | ccgtctc |

SEQ ID NO: 191 Human CXorf21 Amino Acid Sequence (NP_079435.1)

| | |
|---|---|
| 1 | mlsegylsgl eywndihwsc asyneqvage keeetnsvat lsyssvdetq vrslyvscks |
| 61 | sgkfissvhs resqhsrsqr vtvlqtnpnp vfespnlaav eicrdasret ylvpsscksi |
| 121 | cknyndlqia ggqvmainsv ttdfpsessf eygpllksse iplpmedsis tqpsdfpqkp |
| 181 | iqryssywri tsikeksslq mqnpisnavl neyleqkvve lykqyimdtv fhdssptqil |
| 241 | aselimtsvd qislqvsrek nletskardi vfsrllqlms teiteistps lhisqysnvn |
| 301 | p |

SEQ ID NO: 192 Mouse CXorf21 cDNA Sequence (NM_001163539.1; CDS: 166-
1062)

| | |
|---|---|
| 1 | ctcttgttcc ataggatcag tgatactaag aagggggcag atcttagtag cctcttcagt |
| 61 | gagagacttg agagcttttt gaaaagagcc ctgtctggct agttcttctc tattagatca |
| 121 | gtcattgtgt tgctgcagga tccaaggagc agactccggt gaagaatgct atcagaagga |
| 181 | tatctcagtg gacttaccta ctggaatgac attcattgga attgtgcatc ttataatgaa |
| 241 | ccggtggctg gggaccaagg caaagagaca agttctgttg ctgctctttc atattcctct |
| 301 | gtggatgaaa cacaagttca aagtctttat gtgagctgca aatcctctgg gaagtttatt |
| 361 | tcatcagtgc atgcaagggc gagtcagcac agcagaagcc agagcagaac agtgctgcag |
| 421 | gcaaacagca accctgtatt tgaaagtcca actttagctg cagttggtat atgcagagat |
| 481 | gtgatcaggg agacctactt ggttccacct tcttgtaaaa gtatttgcaa aaattacaac |
| 541 | gacttacata ttgcaggggg acaggtgatg gccattaact cagtaatggc aaatttcccc |
| 601 | tctgagagca gctttgaaga tggtcctttg ctaaagtcat ctgagatttc tttgtccatg |
| 661 | gaggattcca cttccactca gctcactgaa cttcccctca aacctatcca gcggtactca |
| 721 | tcctactgga ggataaccag catcaaagag aaaagcagcc tgcaaatgca gaagcctatt |
| 781 | tcaaatgcag tgctcaatga gtacctggag cagaaggtgg tggaattgta taagcaatat |
| 841 | attatggaca ctgtgtttca tgacagttct cctacccaga ttctggcatc agaattcatc |
| 901 | atgacgaatg tagatcaaat tagtcttcaa gtgtctaaag agaagaacct ggacacttca |
| 961 | aaagtcaagg acatagttat tagccacctg ttgcagttgg tatcatctga gatcagcacc |
| 1021 | cctagtcttc atatttctca gtatagcaat ataactccat agagaaggct cctgtcaatt |
| 1081 | ccccttattc ctatgaatcc caaggcacta ttgtattaat ttgatcagag gatgtgatgg |
| 1141 | agggaattct ggaagggaat taacaactag taaaggacac tttgagaaat cacatatgga |
| 1201 | aaggaacagg aagaggaacc aaaagaatgg tttcaaggac aagtttatac agaatcagag |
| 1261 | caaaattaaa aatgatcaca gagagaagga aggtactgtg attcaatttt aggagttctg |
| 1321 | aaactggaag ttaagccaaa tcaggaacaa aaggaaacac tatggcaaac tagaaagtcg |
| 1381 | atggatttga gaattgggaa ttaaacacct gtttcctatt tagatacatt tatctaggtt |
| 1441 | gtttcttctg gaaagaaatg gactacaaga tggttgttaa attcaggtgg actgacattt |
| 1501 | ttccaagtat aagcttgtat ttcttttcct ctgtgctata tcagtaagtc atgtagatat |
| 1561 | actctttata aatattaaat gaatgacaaa caaatctcag ttgtttaaac attttttttt |
| 1621 | tttgcaacaa ggctctatta gctcaggctg gttttacaat ccttacatag ccgatgatct |
| 1681 | tgaattcctt attctcttgc ttctatccca caagtgctgg tattataggt atgtatcatc |
| 1741 | aaatctggat gtgtttttat ttgaaatgaa aaggttcagc cacaagacta tgacttaaat |
| 1801 | ctcctaattc tatttagtga tagagatagc agtgtttcaa cggaaaacct cctattgtga |
| 1861 | acattgaagc attttatttc actgagtgtg attttcatgg tgctatgagg ttcaaaagga |
| 1921 | ttatgaatac tttgcccccc atttcccttt ttcatgtgtt cttctgacct ggagccagaa |
| 1981 | aagggtcttc tgattgttct atacaagacc aaggactcca taatttcaat ttctgtatat |
| 2041 | ttgtttacta aaacagtagc tttacatgtg tattactgtc ttgatctttt attcagctca |
| 2101 | ggacttagaa aactatgagc ctttggtcaa atctaacttt atgatagctt tggactctcg |
| 2161 | tgagctaagg ataggttttc cttttctctt ttcttttttc cctttctttt ccttttttcc |
| 2221 | ttttcttttt cttgagggag ggcctcttta ttgtatagct ccagctgtct ggaactccct |
| 2281 | atgtagacca ggctggtgtc taacacacag agatttgtct gcctgtgctt cccacatgct |
| 2341 | aggaggatta agggcatgtg ccattatatc acacttggca tgttttatta tttttaattt |
| 2401 | taatatattt taaaagccca aaagcttaag ttaaaaatac tttagagcat atgaaaatta |
| 2461 | tatgaaattt aaaattgaaa ttcagtgtat ataaactaga ctttatttgt ttatttattt |
| 2521 | atttatttat ttattggcag tgctggtgat ggaattcaag gccttattca tgacaggcaa |
| 2581 | gttctataac atattccatg ctgcctacta aattttattt gtttgtttat attttatata |
| 2641 | gtttgtagtt gcttttacat aagaatggtc gaacagacat gacggagaac atatgacaca |
| 2701 | acaaagctta atatttcttt ttaccatctg gctgtttaca gtgatgtttg ccaactctgt |
| 2761 | tcaatgacag tgtcttgccc aaacttgatt taggttttcc aggaagcaga ctttcgaaag |
| 2821 | gacattgggg agcattttta tccttatggg gaataggttc cctctgtgat ggaatgaggg |
| 2881 | aagcaaatcc agatgttacc agaaagagtt agggtgaatc ttcaaaatag ttccaaattg |
| 2941 | aaacaagtga gtaggcgtct gttctcaagc gtctaagtgc aaggtgcata ccttgactga |
| 3001 | tggccacagg tctatggagt ggagtggagt ggagtggagt ggagtggagt gaatagctgc |
| 3061 | agcctctggt caggagcctg gtgtctagga gcatggaaaa aggcctacca ttccagtgca |
| 3121 | ggctaaggtc acctgctggg ttaccggggt tttaaaccta gctcaaccag aaaccaggct |
| 3181 | gcctctcata gtcccacaac taatgaccca aagtccatta catccacacc cagccaaatt |
| 3241 | ttctactatc attcttgaaa ttatgtgagg ttctataact gtacataaat aaataaataa |
| 3301 | aagcactgca gaaatctgaa atggctttag taaaattttc tgccacaatg ggactacaac |
| 3361 | agtttagatt cagaaattca gtcttccttt ttttcttttt aaaaaatttt attgggttca |
| 3421 | ttgattctac caagactagt ctgcacaggg gagagtgtgt cactctatga aagactaata |
| 3481 | tttcctaaaa ttgtgaggca gagaaaattc tttccttaag ttgcttctgt gtggtgtttg |
| 3541 | ttatggcaat aagaaattaa tcgagccatc ttagttccca gatccctctg agactatctg |
| 3601 | cacaggtgag agtatggact acagaagcta acagcttccg ggacagaccc tgtttcaggt |
| 3661 | tctcatcttc tgccaggagg cagatctgat tgccagatat ctgtgaacct ttcctgcaag |
| 3721 | aggagagctt gcatgcagtg agtgctctga ccactgaaac tcagaagaga gctagtcccc |
| 3781 | caggtctgct gatagagcgt aacagaatca cctgaggaac aagctctaac cagag |

SEQ ID NO: 193 Mouse CXorf21 Amino Acid Sequence (NP_001157011.1)

| | |
|---|---|
| 1 | mlsegylsgl tvwndihwnc asynepvagd qgketssvaa lsyssvdetq vqslyvscks |
| 61 | sgkfissvha rasqhsrsqs rtvlqansnp vfesptlaav gicrdviret ylvppscksi |
| 121 | ckhyndlhia ggqvmainsv manfpsessf edgpllksse islsmedsts tqltelplkp |
| 181 | iqryssywri tsikeksslq mqkpisnavl neyleqkvve lykqyimdtv fhdssptqil |
| 241 | asefimtnvd qislqvskek nldtskvkdi vishllqlvs seistpslhi sqvsnitp |

SEQ ID NO: 194 Human CD48 Transcript Variant 1 cDNA Sequence (NM_001778.3;
CDS: 89-820)

| | |
|---|---|
| 1 | gtttggtaag ttccgttttt agccccggcc tttttctagc caggctctca actgtctcct |
| 61 | gcgttgctgg gaagttctgg aaggaagcat gtgctccaga ggttgggatt cgtgtctggc |
| 121 | tctggaattg ctactgctgc ctctgLcact cctggtgacc agcattcaag gtcacttggt |
| 181 | acatatgacc gtggtctccg gcagcaacgt gactctgaac atctctgaga gcctgcctga |
| 241 | gaactacaaa caactaacct ggttttatac tttcgaccag aagattgtag aatgggattc |
| 301 | cagaaaatct aagtactttg aatccaaatt taaaggcagg gtcagacttg atcctcagag |
| 361 | tggcgcactg tacatctcta aggtccagaa agaggacaac agcacctaca tcatgagggt |
| 421 | gttgaaaaag actgggaatg agcaagaatg gaagatcaag ctgcaagtgc ttgaccctgt |
| 481 | acccaagcct gtcatcaaaa ttgagaagat agaagacatg gatgacaact gttatctgaa |
| 541 | actgtcatgt gtgatacctg gcgagtctgt aaactacacc tggtatgggg acaaaaggcc |
| 601 | cttcccaaag gagctccaga acagtgtgct tgcaaccacc cttatgccac ataattactc |
| 661 | caggtgttat acttgccaag tcagcaattc tgtgagcagc aagaatggca cggtctgcct |
| 721 | cagtccaccc tgtaccctgg cccggtcctt tggagtagaa tggattgcaa gttggctagt |
| 781 | ggtcacggtg cccaccattc ttggcctgtt acttacctga gatgagctct tttaactcaa |
| 841 | gcgaaacttc aaggccagaa gatcttgcct gttggtgatc atgctcctca ccaggacaga |
| 901 | gactgtatag gctgaccaga agcatgctgc tgaattatca acgaggattt tcaagttaac |
| 961 | ttttaaatac tggttattat ttaattttat atccctttgt tgttttctag tacacagagc |
| 1021 | tatagagata cacatgcttt tttcccaccc aaaattgtga caacattatg tgaatgtttt |
| 1081 | attatttttt aaaataaaca tttgatataa ttgtcaatta actgaaaaaa aaaaaaaaaa |
| 1141 | aaaaaaaaaa aaaaa |

SEQ ID No: 195 Human CD48 Isoform 1 Amino Acid Sequence (NP_001769.2)

| | |
|---|---|
| 1 | mcsrgwdecl alellllpls llvtsiqghl vhmtvvsgsn vtlniseslp enykqltwfy |
| 61 | tfdqkivewd srkskyfesk fkgrvrldpq sgalyiskvq kednstyimr vlkktgneqe |
| 121 | wkiklqvldp vpkpvikiek iedmddncyl klscvipges vnytwygdkr pfpkelqnsv |
| 181 | lettlmphny srcytcqvsn svsskngtvc lsppctlars fgvewiaswl vvtvptilgl |
| 241 | llt |

SEQ ID NO: 196 Human CD48 Transcript Variant 2 cDNA Sequence
(NM_001256030.1; CDS: 89-847)

| | |
|---|---|
| 1 | gtttggtaag ttccgttttt agccccggcc tttttctagc caggctctca actgtctcct |
| 61 | gcgttgctgg gaagttctgg aaggaagcat gtgctccaga ggttgggatt cgtgtctggc |
| 121 | tctggaattg ctactgctgc ctctgtcact cctggtgacc agcattcaag gtcacttggt |
| 181 | acatatgacc gtggtctccg gcagcaacgt gactctgaac atctctgaga gcctgcctga |
| 241 | gaactacaaa caactaacct ggttttatac tttcgaccag aagattgtag aatgggattc |
| 301 | cagaaaatct aagtactttg aatccaaatt taaaggcagg gtcagacttg atcctcagag |
| 361 | tggcgcactg tacatctcta aggtccagaa agaggacaac agcacctaca tcatgagggt |
| 421 | gttgaaaaag actgggaatg agcaagaatg gaagatcaag ctgcaagtgc ttgaccctgt |
| 481 | acccaagcct gtcatcaaaa ttgagaagat agaagacatg gatgacaact gttatctgaa |
| 541 | actgtcatgt gtgatacctg gcgagtctgt aaactacacc tggtatgggg acaaaaggcc |
| 601 | cttcccaaag gagctccaga acagtgtgct tgaaaccacc cttatgccac ataattactc |
| 661 | caggtgttat acttgccaag tcagcaattc tgtgagcagc aagaatggca cggtctgcct |
| 721 | cagtccaccc tgtaccctgg gtaagaagga tccctgggag ctgagggggg cacagggtaa |
| 781 | ctggagttgt tttgaacaaa gaaaggctgg gggtcctatt cagcctcctt gcacagtgtg |
| 841 | gtggtgaatc cctaaggtgt ctgggagagc tgggagacgt gggttctgcc accagctcta |
| 901 | ccaccacctc ccagccagct tacctcaact tcgtgggggc tcagtgttct cacctgcaaa |
| 961 | ggacgtttgg gagagatctc tgatactcct cttccctctc ccgctctaac aaagcatagt |
| 1021 | cctaacatct gaggccaggg tcatcataga gtagactgaa acatcagggt gagcagggag |
| 1081 | aaggaagggc aagtgggcga gcagctgtct agaggggctt cattagacag ccgaagtcag |
| 1141 | ccaaggaaag agggaccgag gtcattagac cgccaaagtc agccagggaa agagggactg |
| 1201 | aggagacggg cctgagagag gccgtcgagg aggcgtgaga gcctgagcct caggcgaagc |
| 1261 | ttctcctccc cagcctgatg ttcctagatg aacttaggaa gccagattcc cctgtctcct |
| 1321 | gggaggatcc actcatgagt gtcacacctg gctctagatc aggcctacac tggtgctagc |
| 1381 | atgggacagc taaggccatg ggttttagag tcagtcatac ctggggtcac ttctaggact |
| 1441 | gtcacttact agctaaacaa gttacttagc ttccccaagt catgttcttc ctaaataaag |
| 1501 | gacaaaataa cagttcctat aaaaaaaaaa aaaaaaa |

SEQ ID NO: 197 Human CD48 Isoform 2 Amino Acid Sequence (NP_001242959.1)

| | |
|---|---|
| 1 | mcsrgwdscl alellllpls llvtsiqghi vhmtvvsgsn vtlniseslp enykqltwfy |
| 61 | tfdqkivewd srkskyfesk fkgrvrldpq sgalyiskvq kednstyimr vlkktgnege |
| 121 | wkiklqvldp vpkpvikiek iedmddncyl klscvipges vnytwygdkr pfpkelqnsv |
| 181 | lettlmphny srcytcqvsn svsskhgtvc lsppctlgkk dpwelrgaqg nwscfeqrka |
| 241 | ggpiqppctv ww |

SEQ ID NO: 198 Mouse CD48 Transcript Variant 1 cDNA Sequence (NM_007649.5;
CDS: 103-825)

| | |
|---|---|
| 1 | atacgacttc cggttttggg ttttgcttcc tgattgaagg gcaggcgccc tgacttctct |
| 61 | tacagttgtc tccagtgttc tggggaagct tctctaagta ttatgtgctt cataaaacag |
| 121 | ggatggtgtc tggtcctgga actgctactg ctgcccttgg gaactggatt tcaaggtcat |
| 181 | tcaataccag atataaatgc caccaccggc agcaatgtaa ccctgaaaat ccataaggac |
| 241 | ccacttggac catataaacg tatcacctgg cttcatacta aaaatcagaa gattttagag |
| 301 | tacaactata atagtacaaa gacaatcttc gagtctgaat ttaaaggcag ggtttatctt |
| 361 | gaagaaaaca atggtgcact tcatatctct aatgtccgga aagaggacaa aggtacctac |
| 421 | tacatgagag tgctgcgtga aactgagaac gagttgaaga taaccctgga agtatttgat |
| 481 | cctgtgccca agccttccat agaaatcaat aagactgaag cgtcgactga ttcctgtcac |
| 541 | ctgaggctat cgtgtgaggt aaaggaccag catgttgact atacttggta tgagagctcg |
| 601 | ggacctttcc ccaaaaagag tccaggatat gtgctcgatc tcatcgtcac accacagaac |
| 661 | aagtctacat tttacacctg ccaagtcagc aatcctgtaa gcagcaagaa cgacacagtg |
| 721 | tacttcactc taccttgtga tctagccaga tcttctggag tatgttggac tgcaacttgg |
| 781 | ctagtggtca caacactcat cattcacagg atcctgttaa cctgacaaga actcttctca |
| 841 | cccaagaagg caacttggaa gcacagagtc ttgccttcat ccctagcagt gttcctagcc |
| 901 | agcgaagcaa ctctggctct attggacaaa ggaaaatgtg ttactgaacg tctgcgagag |
| 961 | tttgcatgca tgctctatga aacaagcaca ggaccttgta cagtgctcca ccactgacct |
| 1021 | gtgtgcccag tcctttacaa agatttcaaa tcaacctttt aaaaactgtg cataatatct |
| 1081 | aattttatat accctagttg tttcccaaca tatattaaag ataaatgcat tctttttacc |
| 1141 | aaaatgtgac tatattattt tcatgttttc atatctcttt ttaaaataaa ttcttttaaa |
| 1201 | aaact |

SEQ ID NO: 199 Mouse CD48 Isoform 1 Amino Acid Sequence (NP_031675.1)

| | |
|---|---|
| 1 | mcfikqgwcl vlellllplg tgfqghsipd inattgsnvt lkihkdplgp ykritwlhtk |
| 61 | nqkileynyn stktifesef kgrvyleenn galhisnvrk edkgtyymrv lretenelki |
| 121 | tlevfdpvpk psieinktea stdschlrls cevkdqhvdy twyessgpfp kkspgyvldl |
| 181 | ivtpqnkstf ytcqvsnpvs skndtvyftl pcdlarssgv cwtatwlvvt tliihrillt |

SEQ ID NO: 200 Mouse CD48 Transcript Variant 2 cDNA Sequence
(NM_001360767.1; CDS: 103-558)

| | |
|---|---|
| 1 | atacgacttc cggttttggg ttttgcttcc tgattccagg gcaggcgccc tgacttctct |
| 61 | tacagttgtc tccagtgttc tggggaagct tctctaagta ttatgtgctt cataaaacag |
| 121 | ggatggtgtc tggtcctgga actgctactg ctgcccttgg gaactggatt tcaaggtcat |
| 181 | tcaataccag atataaatgc caccaccggc agcaatgtaa ccctgaaaat ccataaggac |
| 241 | ccacttggac catataaacg tatcacctgg cttcatacta aaaatcagaa gattttagag |
| 301 | tacaactata atagtacaaa gacaatcttc gagtctgaat ttaaaggcag ggtttatctt |
| 361 | gaagaaaaca atggtgcact tcatatctct aatgtccgga aagaggacaa aggtacctac |
| 421 | tacatgagag tgctgcgtga aactgagaac gagttgaaga taaccctgga agtatttgcc |
| 481 | agatcttctg gagtatgttg gactgcaact tggctagtgg tcacaacact catcattcac |
| 541 | aggatcctgt taacctgaca agaactcttc tcacccaaga aggcaacttg ccagcacaga |
| 601 | gtcttgcctt catccctagc agtgttccta gccagcgaag caactctggc tctattggac |
| 661 | aaaggaaaat gtgttactga acgtctgcga gagtttgcat gcatgctcta tgaaacaagc |
| 721 | acaggacctt gtacagtgct ccaccactga cctgtgtgcc cagtccttta caaagatttc |
| 781 | aaatcaacct tttaaaaact gtgcataata tctaatttta tataccctag ttgtttccca |
| 841 | acatatatta aagataaatg cattcttttt accaaaatgt gactatatta ttttcatgtt |
| 901 | ttcatatctc tttttaaaat aaattctttt aaaaaact |

SEQ ID NO: 201 Mouse CD48 Isoform 2 Amino Acid Sequence (NP_001347696.1)

| | |
|---|---|
| 1 | mcfikqgwcl vlellllplg tgfqghsipd inattgsnvt lkihkdplgp ykritwlhtk |
| 61 | nqkileynyn stktifesef kgrvyleenn galhisnvrk edkgtyymrv lretenelki |
| 121 | tlevfarssg vcwtatwlvv ttliihrill t |

SEQ ID NO: 202 Human CD84 Transcript Variant 1 cDNA Sequence
(NM_001184879.1; CDS: 80-1117)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctggccg gaagcagctg gaaaagactc agaaatcttc acagtgaatg ggattctggg |
| 181 | agagtcagtc actttccctg taaatattca agaaccacgg caagttaaaa tcattgcttg |
| 241 | gacttctaaa acatctgttg cttatgtaac accaggagac tcagaaacag cacccgtagt |
| 301 | tactgtgacc cacagaaatt attatgaacg gatacatgcc ttaggtccga actacaatct |
| 361 | ggtcattagc gatctgagga tggaagacgc aggagactac aaagcagaca taaatacaca |
| 421 | ggctgatccc tacaccacca ccaagcgcta caacctgcaa atctatcgtc ggcttgggaa |
| 481 | accaaaaatt acacagagtt taatggcatc tgtgaacagc acctgtaatg tcacactgac |
| 541 | atgctctgta gagaaagaag aaaagaatgt gacatacaat tggagtcccc tgggagaaga |
| 601 | gggtaatgtc cttcaaatct tccagactcc tgaggaccaa gagctgactt acacgtgtac |
| 661 | agcccagaac cctgtcagca acaattctga ctccatctct gcccggcagc tctgtgcaga |
| 721 | catcgcaatg ggcttccgta ctcaccacac cgggttgctg agcgtgctgg ctatgttctt |
| 781 | tctgcttgtt ctcattctgt cttcagtgtt tttgttccgt ttgttcaaga gaagacaagg |
| 841 | taggattttc ccagaaggtt cctgcttgaa caccttcact aagaaccctt atgctgcctc |
| 901 | aaagaaaacc atatacacat atatcatggc ttcaaggaac acccagccag cagagtccag |
| 961 | aatctatgat gaaatcctgc agtccaaggt gcttccctcc aaggaagagc cagtgaacac |
| 1021 | agtttattcc gaagtgcagt ttgctgataa gatggggaaa gccagcacac aggacagtaa |
| 1081 | acctcctggg acttcaagct atgaaattgt gatctaggct gctgggctga attctccctc |
| 1141 | tggaaactga gttacaacca ccaatactgg caggttccct ggatccagat cttctctgcc |
| 1201 | caactcttac tgggagattg caaactgcca catctcagcc tgtaagcaaa gcaggaaacc |
| 1261 | ttctgctggg catagcttgt gcctaaatgg acaaatggat gcataccctt cctgaaatga |
| 1321 | ctcccttctg aatgaatgac aaagcaggtt acctagtata gttttcccaa acttcttccc |
| 1381 | atcatagcac atgtagaaaa taatattttt atggcacact gggataaaca agcaagattg |
| 1441 | ctcacttctg gaagctgcat atgactagag gcctcttgtg actggaggta acaaccctgc |
| 1501 | ccagtaactg tgggagaagg ggatcaatat tttgcacacc tgtaataggc catggcacac |
| 1561 | cagccaagat gctctgctca cagtcagtat gtgtgaagat ccctggtgcg tggccttcac |
| 1621 | cacgcatctt gagcaaatta ggaaaatgta cccttcgctt gaggcagatg cagcccttcc |
| 1681 | cccgagtgca tggcttggag agcagaatgt gggctgcata taagcacact catccctttg |
| 1741 | tctgggaatc tttgtgcagg gcataacagg cttagtaagt ccaaacacag atgacagtgc |
| 1801 | tgtgtgggtc tctgtcagag ttgtggctct cagccatgta gacacactct ccaaatggag |
| 1861 | tgttggaaaa tgttctttct gcagggtcta gagactgctg ggacactttt cttggagtgc |
| 1921 | tacttcagaa gccttatagg attttctttc tggccaagat ttccttctgt atcactccaa |
| 1981 | gcagcctcag cagaagaagc agccatgccc agtattccca ctctccaaaa ggaactgacc |
| 2041 | agcttatatt tctcacactt ctggggaact gggtataatc caaccatcaa aatagaagac |
| 2101 | cttgcaagaa gcagagtcat tctccagaag gaacttggga gatgatggtg cagatgatga |
| 2161 | aactgggttc atcccagttc caaagactca gagaactaga gtttaagctg aggcagagtg |
| 2221 | ccgccaccct ggcatgcccc acaaacagat caccagccag cttacacagg cattaactct |
| 2281 | cctcaatgag gaagaatcat tcacaactga gcaagacatt catatgatca tttaaggaag |
| 2341 | tgtttccctt atgtgttagc aagtataatc ggctaactcc taaatcccaa tgaatagtcc |
| 2401 | taggctggac agcaatgggc tgcaattagg cagataaaga catcagtccc agtaaatgaa |
| 2461 | tccatagact catctagcac caactaccat tagcactatg ttaggagctg caaggcccca |
| 2521 | aagtagaaga tgtgcataat gtctgctctt gtgtagctca ggagacaatt ccagcacaga |
| 2581 | cactacagtt aacgctgaac tgcagctgca agtaatagca tgaacagtca gaaaaatacc |
| 2641 | ttatgagggg gcagggctga agctgggcct tgaaggatgg atgaaatttg gatagagaat |
| 2701 | gaggaagaca gagggcctcc aagtgagaga agcatgaaaa atgagcaggg gcctggatca |
| 2761 | gtggggtgta ttcagagcac ctactccaga gcaccatgca tgctcacagt cccttgccta |
| 2821 | tgtgtggcag agtgtcccag ccagatgtgt gccctcaccc catgtccatt tacatgtcct |
| 2881 | tcaatgccca cctcaaaagg tacctcttct gtaaagcttt ccctggtatc aggaatcaaa |
| 2941 | attaatcagg gatcttttca cactgctgtt ttttcctctt tggtccttct atcactaaaa |
| 3001 | ctcatctcat tcagccttac agcataacta attatttgtt ttcctcacta cattgtacat |
| 3061 | gtgggaatta cagataaacg gaagccggct ggggtggtgg ctcacgcctg taatcccaac |
| 3121 | actttgggag gccaaggcag gcggatcacc tgaggtcagg agttcgagat tagtctggcc |
| 3181 | aacatggtga aaccccatct ctactaaaaa tacgaaatta gccaggtgtg gtggcacaca |
| 3241 | tctgtagtcc cagctactct ggaggctgag acaggagaat cgcttgaacc caggaagtgg |
| 3301 | aggttgcagt gagctgagat cacaccactg cactccagcc tgggagagac agagtgagac |
| 3361 | tccatctcga aaaaaaaaaa aagatagaag ccaataagca tggtgcaatc aaattctggc |
| 3421 | aagcattaaa tatcaggatg cagctgggca cggtggctca cgcctgtaat cccagcactt |
| 3481 | tgggaggcca aggtgggcgg atcacttgag gtcaggaatt tgagaggatc ctggccagca |
| 3541 | tggcaaaacc ccatctgtac ttaaaataca aaaaaattag ctgggcgtgg tggtgcacac |
| 3601 | ctgtaatccc agctacttgg gaggctgagg tgggagaatt gcttgaacct gggaggtgga |
| 3661 | ggttgcagtg agctgagatc ctgccactgc actccaggct gggcaacaga gtgagaccat |
| 3721 | gtctcaaaaa ataaaaataa aataaaataa tatcaggatg catacatcag aggctgttcc |
| 3781 | tagtgtaaag gcactttgga gggagaagac tttcagagtt aggcagacca actaagaggt |
| 3841 | cagctgaagc acctaaccag ttgtaaggag gtgaaagaca gcaccccaag aagagacgtg |
| 3901 | caggaaggag gaaagaggct tggtcataaa ggatggagga attccaaagt gacactgaac |
| 3961 | aggctgcgtt tatcctaaaa taaaaccact cctcactctg tggatgcgtt gaagactcat |
| 4021 | tcccaaacat ctttattctc taacttgccc tcttcctctt cctaatatgc tcactcaagt |
| 4081 | aaaattacta gtgtcctaat gcccctatgc atattgtcaa aaataaaaat cagaagcagg |
| 4141 | ttagatctgt taggtcttcc agaagagcaa acctgggatg aagccagagc ccaggaattc |
| 4201 | tgaaggtagc ctttggactc aggacaccct actcttgtct ctcctctcag tttctctgct |
| 4261 | atgaatctcc tgattcatga acacgttatc tgttcaccct tctctctagg tcttagttct |
| 4321 | tagattttcc ttctgtaaaa tgcatgtgat cttattttcc cctccacaac tttccagatg |
| 4381 | aactagactg tgaccaagag gtctataaaa tcaaagcatc atggaacagg atcttgtatc |
| 4441 | agaccaaagt gtgccagttt ttaaaaatgt gcatcaaaat ggaagtctca gagacagagc |
| 4501 | cctctggtgg aaagttctag taggttagga cagtcctgcc tgcagacacc ttgggcttta |
| 4561 | ctgagggact caactgagaa aatgaggaat gttgcagctc atgattctta gaagaagaaa |
| 4621 | gtgaagcttg tttaaaatat gatttaaaaa atctgtagaa cactgtaaac tacacaggct |
| 4681 | atgagggaat agcctggttg ggccagcttg gaaatcgggc acaggcagga aggggcctgt |
| 4741 | ctggtttggg ccgtgtccac agagagcact tcttaggtcc tgcctggaga gaaggaatgg |
| 4801 | ctgggctata ttttcttcca gactcattat ttttcttctg tttgactttt ctctgaattt |
| 4861 | cccttgattt gtataaattt tctcaataat tagtgacagt gtctactgat tgtaaaatga |
| 4921 | agcttgaagg ccaggcgcag tggctcatgc ctgtaatcct agaattttgg gaggccaagg |
| 4981 | tgggtggatc acaaggagtt cgagaccagc ctggccaagg ttgtgaaacc gcgtctctac |
| 5041 | taaaaataca aaaaaattag grgggcatgg tggcacgtgc ctgtagtccc agctactcag |
| 5101 | gaagctgagg caacagaatc acttgaacct gggaggtgga ggttgcagtg agccgagatc |
| 5161 | acgccactgc actccagcct gggcgagaga gtgagactcc gcctcaaaaa aaaaaaaaaa |
| 5221 | aaaaaaagtt tgaagaacaa agacaataag aggaaatata atgagtggtc ataaatgtgg |
| 5281 | gctctgacag tagagtgcct gggtctgcat cctggtttct tagtcatgtg accttaggca |
| 5341 | agttacttta acctcgctgt acctcaggtt gtccatctgt aaaatgggga taataatagt |
| 5401 | gcctaccttt taaggttgat gtggggatta aatgaggtgt tgctcataca ggaatgtgcc |
| 5461 | tgtgcatggc aaagttcggg aaatttttta taagctgttc taggcctgaa atcttcagaa |
| 5521 | gatgctaatc taaattcatg aaataagctt cttacaacag aaatgctgct agtattatgc |
| 5581 | aaaattaatg ttgtatatca aacttttaac tctcatccct ccttattcag atatattttg |
| 5641 | ttataagcaa tgtttgttcc cctcgttatt ataccacagt ctacttacct gatgctatat |
| 5701 | ctgcctcccc agttagactg agagaacagg ggatatacct aaataataat aataataata |
| 5761 | ataataataa ataataatgg agagctcctt gaagataggg agcctgtaag aatcattgag |
| 5821 | ggcttatttt gtataccaac tgctaaacta gatgcttcat acattgttgt caatactcat |
| 5881 | gacagccttg taaagtagaa attaattctt ccagttaaca ctaaggctga catatgaata |
| 5941 | ccttggcaaa tctggaaagc tgggaagaca gtatttgaat tcaagacttc ttgtcaccaa |
| 6001 | gggccatgca cttgtactct gccatgtggc ccttttttac ctcctgtgga ttctccctac |
| 6061 | ctggtacttg gccttaggtg tacacacacc tggcactttg cttgacacat aataggtgga |
| 6121 | ccacaaatat ctactaaatg aatatttgca tatagtaata ttttaaggta ctaaaagcag |
| 6181 | ctcaaagtaa atatattaat atattaattc cattgctatc tggataacca ctcaactttc |
| 6241 | ctgctgaaaa tgcccattta attaaagaag gttggataga gctctctata tgcattttgg |
| 6301 | acaggcaggg gtttcaggtc ataaacattc tgatgagtta atataaaata agagaaactg |
| 6361 | taaatttcca ctactaaaaa tcacaaaaat aacagaaaca aaagaagaga taagaatttg |
| 6421 | gggaattgtg ctgaacaatt tagtggttaa aaaaaacaac tgtgcatgtt tagacttaaa |
| 6481 | taagccccca tccaagtgtg aggggtccag taatttttca aaacatatga aagtgttaat |
| 6541 | acatttcgac aaaggaccat taaaaaagtc ctgaattctg acttgaggga ggaaagtaat |
| 6601 | gactaataca ttctctagag acttgcagac tttgggaatt cataaaggaa tggatgataa |
| 6661 | ttattaactg ttgctggctg attgcccaga cagttctcaa cagccctgta caagtctctg |
| 6721 | ggtttgggat ggatcaattc tgagactgga aaatggccaa atctttgcaa atgagaaata |
| 6781 | tttttcttat aagttcttat tgtaggcaaa taattacata gattattcat cagagaattt |
| 6841 | ttaaatgctc ataatctcaa ctctttcatt tacaacttgt atttccaata gtttatgggt |
| 6901 | catctctgca tagatgtcag aagtcacctc aagtttagcg tgtccaaaat ctaactcaca |
| 6961 | ggtctgtttc tgacctccca acttgctttc cttgtgtttt tcctatgcta atgatccacc |
| 7021 | ataatcaaaa taattaacat ttatccagtg cctactatgt actattccct gtcctgtttt |
| 7081 | acatttactc atttaaagtc cataagaaac attaaatctc atctgccttc tgaagaagat |
| 7141 | acaaccatgc tctcttttac aaagtaggaa actgggtcdc agaaaggtga agtctttaag |
| 7201 | gctgaatcac agtagctcat cctagtaaat agaaaagcca ggattcaact ccaggggctg |
| 7261 | ggtgcagaac tgctattctt cactgcttca ccaatcagca gctacccaag gcagaaaact |
| 7321 | ttttcatcct tggctccttc attctccctg tcaccccaga tcccctctac atctagtcag |
| 7381 | agaataggtc ctgtcaattc caacttctct atatggatcc tctcaggcat gtgcccttaa |
| 7441 | ttggcctaat tctctaatac accttccctc tacatgatca ctccctcaga tcattgcttt |
| 7501 | atcacgtgtt acctgggttg ctattacata aagagcaatc tttctaaaat gaggatctta |
| 7561 | tcacttcact tccacactaa aatgtttttc ctggggaacc acactcctta gcaatctgac |
| 7621 | ccatcagacc ttccaggctg tctcctgcct gctccataag gctccagcca cacagaatta |
| 7681 | tcatgggccc acacacccac caaatcctcc catgcctttg cccatgttgt ctgggatgcc |
| 7741 | cttctctccc ttctgtctac atcaagcatc agactgaata tccctcttgt gcggccttct |
| 7801 | aaaacctccc gtccaaagcg aaatatattg ccctctattt atacttttac agcatttggc |
| 7861 | acacaagtac agagtagtag ctttttatca cattctctga taattatata gatatggtat |
| 7921 | ttcttagctc tctctccaac tggctaataa gttgcttttt gtctgagtgc ctaattttgt |
| 7981 | gttttgtgtc tgagtgcctc agttcctcaa aaaaaggttt tttgattagt tcattattca |
| 8041 | tttgaacatg gaaattatgc tcactagtgg caaatgccac taaccgtatt ccagaagcta |
| 8101 | ggtgtcatgt ttgcaataag atatattatc ccttctacaa gtcacctttt atttcaggca |
| 8161 | tttgtaaatg cccattaata aagtatggtt cataaatttt accttgtaag tgcctaagaa |
| 8221 | atgagactac aagctccatt tcagcaggac acaataaata ttattttata atgcatctaa |
| 8281 | aaaaaaaaaa aaaaaa |

SEQ ID NO: 203 Human CD84 Isoform 1 Amino Acid Sequence (NP_001171808.1)

| | |
|---|---|
| 1 | maqhhlwill iclqtwpeaa gkdseiftvn gilgesvtfp vniqeprqvk iiawtsktsv |
| 61 | ayvtpgdset apvvtvthrn yyerihalgp nynlvisdlr medagdykad intqadpytt |
| 121 | tkrynlqiyr rlgkpkitqs lmasvnstcn vtltcsveke eknvtynwsp lgeegnvlqi |
| 181 | fqtpedqelt ytctaqnpvs nnsdsisarq lcadiamgfr thhtgllsvl amffllvlii |
| 241 | ssvflfrlfk rrqgrifpeg sclntftknp yaaskktiyt yimasrntqp aesriydeil |
| 301 | qskvlpskee pvntvysevq fadkmgkast qdskppgtss yeivi |

SEQ ID NO: 204 Human CD84 Transcript Variant 2 cDNA Sequence (NM_003874.3;
CDS: 80-1066)

| | |
|---|---|
| 1 | gccggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctggccg gaagcagctg gaaaagactc agaaatcttc acagtgaatg ggattctggg |
| 181 | agagtcagtc actttccctg taaatatcca agaaccacgg caagttaaaa tcattgcttg |
| 241 | gacttctaaa acatctgttg cttatgtaac accaggagac tcagaaacag cacccgtagt |
| 301 | tactgtgacc cacagaaatt attatgaacg gatacatgcc ttaggtccga actacaatct |
| 361 | ggtcattagc gatctgagga tggaagacgc aggagactac aaagcagaca taaatacaca |
| 421 | ggctgatccc tacaccacca ccaagcgcta caacctgcaa atctatcgtc ggcttgggaa |
| 481 | accaaaaatt acacagagtt taatggcatc tgtgaacagc acctgtaatg tcacactgac |
| 541 | atgctctgta gagaaagaag aaaagaatgt gacatacaat tggagtcccc tgggagaaga |
| 601 | gggtaatgtc cttcaaatct tccagactcc tgaggaccaa gagctgactt acacgtgtac |
| 661 | agcccagaac cctgtcagca acaattctga ctccatctct gcccggcagc tctgtgcaga |
| 721 | catcgcaatg ggcttccgta ctcaccacac cgggttgctg agcgtgctgg ctatgttctt |
| 781 | tctgcttgtt ctcattctgt cttcagtgtt tttgttccgt ttgttcaaga gaagacaaga |
| 841 | tgctgcctca aagaaaacca tatacacata tatcatggct tcaaggaaca cccagccagc |
| 901 | agagtccaga atctatgatg aaatcctgca gtccaaggtg cttccctcca aggaagagcc |
| 961 | agtgaacaca gtttattccg aagtgcagtt tgctgataag atggggaaag ccagcacaca |
| 1021 | ggacagtaaa cctcctggga cttcaagcta tgaaattgtg atctaggctg ctgggctgaa |
| 1081 | ttctccctct ggaaactgag ttacaaccac caatactggc aggttccctg gatccagatc |
| 1141 | ttctctgccc aactcttact gggagattgc aaactgccac atctcagcct gtaagcaaag |
| 1201 | caggaaacct tctgctcggc atagcttgtg cctaaatgga caaatggatg catacccttc |
| 1261 | ctgaaatgac tcccttctga atgaatgaca aagcaggtta cctagtatag ttttcccaaa |
| 1321 | cttcttccca tcatagcaca tgtagaaaat aatattttta tggcacactg ggataaacaa |
| 1381 | gcaagattgc tcacttctgg aagctgcata tgactagagg cctcttgtga ctggaggtaa |
| 1441 | caaccctgcc cagtaactgt gggagaaggg gatcaatatt ttgcacacct gtaataggcc |
| 1501 | atggcacacc agccaagatg ctctgctcac agtcagtatg tgtgaagatc cctggtgcgt |
| 1561 | ggccttcacc acgcatcttg agcaaattag gaaaatgtac ccttcgcttg aggcagatgc |
| 1621 | agcccttccc ccgagtgcat ggcttggaga gcagaatgtg ggctgcatat aagcacactc |
| 1681 | atccctttgt ctgggaatct ttgtgcaggg cataacaggc ttagtaagtc caaacacaga |
| 1741 | tgacagtgct gtgtgggtct ctgtcagagt tgtggctctc agccatgtag acacactctc |
| 1801 | caaatggagt gttggaaaat gttctttctg cagggtctag agactgctgg gacacttttc |
| 1861 | ttggagtgct acttcagaag ccttatagga ttttctttct ggccaagatt tccttctgta |
| 1921 | tcactccaag cagcctcagc agaagaagca gccatgccca gtattcccac tctccaaaag |
| 1981 | gaactgacca gcttatattt ctcacacttc tggggaactg ggtataatcc aaccatcaaa |
| 2041 | atagaagacc ttgcaagaag cagagtcatt ctccagaagg aacttgggag atgatggtgc |
| 2101 | agatgatgaa actgggttca tcccagttcc aaagactcag agaactagag tttaagctga |
| 2161 | ggcagagtgc cgccaccctg gcatgcccca caaacagatc accagccagc ttacacaggc |
| 2221 | attaactctc ctcaatgagg aagaatcatt cacaactgag caagacattc atatgatcat |
| 2281 | ttaaggaagt gtttccctta tgtgttagca agtataatcg gctaactcct aaatcccaat |
| 2341 | gaatagtcct aggctggaca gcaatgggct gcaattaggc agataaagac atcagtccca |
| 2401 | gtaaatgaat ccatagactc atctagcacc aactaccatt agcactatgt taggagctgc |
| 2461 | aaggccccaa agtagaagat gtgcataatg tctgctcttg tgtagctcag gagacaattc |
| 2521 | cagcacagac actacagtta acgctgaact gcagctgcaa gtaatagcat gaacagtcag |
| 2581 | aaaaatacct tatgaggggg cagggctgaa gctgggcctt gaaggatgga tgaaatttgg |
| 2641 | atagagaatg aggaagacag agggcctcca agtgagagaa gcatgaaaaa tgagcagggg |
| 2701 | cctggatcag tggggtgtat tcagagcacc tctccagatg caccatgcat gctcacagtc |
| 2761 | ccttgcctat gtgtggcaga gtgtcccagc cagatgtgtg ccctcacccc atgtccattt |
| 2821 | acatgtcctt caatgcccac ctcaaaaggt acctcttctg taaagctttc cctggtatca |
| 2881 | ggaatcaaaa ttaatcaggg atcttttcac actgctgttt tttcctcttt ggtccttcta |
| 2941 | tcactaaaac tcatctcatt cagccttaca gcataactaa ttatttgttt tcctcactac |
| 3001 | attgtacatg tgggaattac agataaacgg aagccggctg gggtggtggc tcacgcctgt |
| 3061 | aatcccaaca ctttgggagg ccaaggcagg cggatcacct gaggtcagga gttcgagatt |
| 3121 | agtctggcca acatggtgaa accccatctc tactaaaaat acgaaattag ccaggtgtgg |
| 3181 | tggcacacat ctgtagtccc agctactctg gaggctgaga caggagaatc gcttgaaccc |
| 3241 | aggaagtgga ggttgcagtg agctgagatc acaccactgc actccagcct gggagagaca |
| 3301 | gagtgagact ccatctcgaa aaaaaaaaaa agatagaagc caataagcat ggtgcaatca |
| 3361 | aattctggca agcattaaat atcaggatgc agctgggcac ggtggctcac gcctgtaatc |
| 3421 | ccagcacttt gggaggccaa ggtgggcgga tcacttgagg tcaggaattt gagaggatcc |
| 3481 | tggccagcat ggcaaaaccc catctgtact taaaatacaa aaaaattagc tgggcgtggt |
| 3541 | ggtgcacacc tgtaatccca gctacttggg aggctgaggt gggagaattg cttgaacctg |
| 3601 | ggaggtggag gttgcagtga gctgagatcc tgccactgca ctccaggctg ggcaacagag |
| 3661 | tgagaccatg tctcaaaaaa taaaaataaa ataaaataat atcaggatgc atacatcaga |
| 3721 | ggctgttcct agtgtaaagg cactttggag ggagaagact ttcagagtta ggcagaccaa |
| 3781 | ctaagaggtc agctgaagca cctaaccagt tgtaaggagg tgaaagacag caccccaaga |
| 3841 | agagacgtgc aggaaggagg aaagaggctt ggtcataaag gatggaggaa ttccaaagtg |
| 3901 | acactgaaca ggctgcgttt atcctaaaat aaaaccactc ctcactctgt ggatgcgttg |
| 3961 | aagactcatt cccaaacatc tttattctct aacttgccct cttcctcttc ctaatatgct |
| 4021 | cactcaagta aaattactag tgtcctaatg cccctatgca tattgtcaaa aataaaaatc |
| 4081 | agaagcaggt tagatctgtt aggtcttcca gaagagcaaa cctgggatga agccagagcc |
| 4141 | caggaattct gaaggtagcc tttggactca ggacacccta ctcttgtctc tcctctcagt |
| 4201 | ttctctgcta tgaatctcct gattcatgaa cacgttatct gttcaccctt ctctctaggt |
| 4261 | cttagttctt agattttcct tctgtaaaat gcatgtgatc ttattttccc ctccacaact |
| 4321 | ttccagatga actagactgt gaccaagagg tctataaaat caaagcatca tggaacagga |
| 4381 | tcttgtatca gaccaaagtg tgccagtttt taaaaatgtg catcaaaatg gaagtctcag |
| 4441 | agacagagcc ctctggtgga aagttctagt aggttaggac agtcctgcct gcagacacct |
| 4501 | tgggctttac tgagggactc aactgagaaa atgaggaatg ttgcagctca tgattcttag |
| 4561 | aagaagaaag tgaagcttgt ttaaaatatg atttaaaaaa factgtagaa actgtaaact |
| 4621 | acacaggcta tgagggaata gcctggttgg gccagcttgg aaatcgggca caggcaggaa |
| 4681 | ggggcctgtc tggtttgggc cgtgtccaca gagagcactt cttaggtcct gcctggagag |
| 4741 | aaggaatggc tgggctatat tttcttccag actcattatt tttcttctgt ttgacttttc |
| 4801 | tctgaatttc ccttgatttg tataaatttt ctcaataatt agtgacagtg tctactgatt |
| 4861 | gtaaaatgaa gcttgaaggc caggcgcagt ggctcatgcc tgtaatccta gaattttggg |
| 4921 | aggccaaggt gggtggatca caaggagttc gagaccagcc tggccaaggt tgtgaaaccg |
| 4981 | cgtctctact aaaaatacaa aaaaattagc cgggcatggt ggcacgtgcc tgtagtccca |
| 5041 | gctactcagg aagctgaggc aacagaatca cttgaacctg ggaggtggag gttgcagtga |
| 5101 | gccgagatca cgccactgca ctccagcctg ggcgagagag tgagactccg tctcaaaaaa |
| 5161 | aaaaaaaaaa aaaaaagttt gaagaacaaa gacaataaga ggaaatataa tgagtggtca |
| 5221 | taaatgtggg ctctgacagt agagtgcctg ggtctgcatc ctggtttctt agtcatgtga |
| 5281 | ccttaggcaa gttactttaa cctcgctgta cctcaggttg tccatctgta aaatggggat |
| 5341 | aataatagtg cctacctttt aaggttgatg tggggattaa atgaggtgtt gctcatacag |
| 5401 | gaatgtgcct gtgcatggca aagttcggga aattttttat aagctgttct aggcctgaaa |
| 5461 | tcttcagaag atgctaatct aaattcatga aataagcttc ttacaacaga aatgctgcta |
| 5521 | gtattatgca aaattaatgt tgtatatcaa acttttaact ctcatccctc cttattcaga |
| 5581 | tatattttgt tataagcaat gtttgttccc ctcgttatta taccacagtc tacttacctg |
| 5641 | atgctatatc tgcctcccca gttagactga gagaacaggg gatataccta aataataata |
| 5701 | ataataataa taataataaa taataatgga gagctccttg aagataggga gcctgtaaga |
| 5761 | atcattgagg gcttattttg tataccaact gctaaactag atgcttcata cattgttgtc |
| 5821 | aatactcatg acagccttgt aaagtagaaa ttaattcttc cagttaacac taaggctgac |
| 5881 | atatgaatac cttggcaaat ctggaaagct gggaagacag tatttgaatt caagacttct |
| 5941 | tgtcaccaag ggccatgcac ttgtactctg ccatgtggcc cttttttacc tcctgtggat |
| 6001 | tctccctacc tggtacttgg ccttaggtgt acacacacct ggcactttgc ttgacacata |
| 6061 | ataggtggac cacaaatatc tactaaatga atatttgcat atagtaatat tttaaggtac |
| 6121 | taaaagcagc tcaaagtaaa tatattaata tattaattcc attgctatct ggataaccac |
| 6181 | tcaactttcc tgctgaaaat gcccatttaa ttaaagaagg ttggatagag ctctctatat |
| 6241 | gcattttgga caggcagggg tttcaggtca taaacattct gatgagttaa tataaaataa |
| 6301 | gagaaactgt aaatttccac tactaaaaat cacaaaaata acagaaacaa aagaagagat |
| 6361 | aagaatttgg ggaattgtgc tgaacaattt agtggttaaa aaaaacaact gtgcatgttt |
| 6421 | agaacttaaa aagcccccat ccaagtgtga ggggtccagt aatttttcaa aacatatgaa |
| 6481 | agtgttaata catttcgaca aaggaccatt aaaaaagtcc tgaattctga cttgagggag |
| 6541 | gaaagtaatg actaatacat tctctagaga cttgcagact ttgggaattc ataaaggaat |
| 6601 | ggatgataat tattaactgt tgctggctga ttgcccagac agttctcaac agccctgtac |
| 6661 | aagtctctgg gtttgggatg gatcaattct gagactggaa aatggccaaa tctttgcaaa |
| 6721 | tgagaaatat ttttcttata agttcttatt gtaggcaaat aattacatag attattcatc |
| 6781 | agagaatttt taaatgctca taatctcaac tctttcattt acaacttgta tttccaatag |
| 6841 | tttatgggtc atctctgcat agatgtcaga agtcacctca agtttagcgt gtccaaaatc |
| 6901 | taactcacag gtctgtttct gacctcccaa cttgctttcc ttgtgttttt cctatgctaa |
| 6961 | tgatccacca taatcaaaat aattaacatt tatccagtgc ctactatgta ctattccctg |
| 7021 | tcctgtttta catttactca tttaaagtcc ataagaaaca ttaaatctca tctgccttct |
| 7081 | gaagaagata caaccatgct ctcttttaca aagtaggaaa ctgggtcaca gaaaggtgaa |
| 7141 | gtctttaagg ctgaatcaca gtagctcatc ctagtaaata gaaaagccag gattcaactc |
| 7201 | caggggctgg gtgcagaact gctattcttc actgcttcac caatcagcag ctacccaagg |
| 7261 | cagaaaactt tttcatcctt ggctccttca ttctccctgt caccccagat cccctctaca |
| 7321 | tctagtcaga gaataggtcc tgtcaattcc aacttctcta tatggctcct ctcaggcatg |
| 7381 | tgcccttaat tggcctaatt ctctaataca ccttccctct acatgctcac tccctcagat |
| 7441 | cattgcttta tcacgtgtta cctgggttgc tattacataa agagcaatct ttctaaaatg |
| 7501 | aggatcttat cacttcactt ccacactaaa atgtttttcc tggggaacca cactccttag |
| 7561 | caatctgacc catcagacct tccaggctgt ctcctgcctg ctccctaagg ctccagccac |
| 7621 | acagaattat catgggccca cacacccacc aaatcctccc atgcctttgc ccatgttgtc |
| 7681 | tgggatgccc ttctctccct tctgtctaca tcaagcatca gactgaatat ccctcttgtg |
| 7741 | cggccttcta aaacctcccg tccaaagcga aatatattgc cctctattta tacttttaca |
| 7801 | gcatttggca cacaagtaca gagtagtagc tttttatcac attctctgat aattatatag |
| 7861 | atatggtatt tcttagctct ctctccaact ggctaataag ttgctttttg tctgagtgcc |
| 7921 | taattttgtg ttttgtgtct gagtgcctca gttcctcaaa aaaaggtttt ttgattagtt |
| 7981 | cattattcat ttgaacatgg aaattatgct cactagtggc aaatgccact aaccgtattc |
| 8041 | cagaagctag gtgtcatgtt tgcaataaga tatattatcc cttctacaag tcacctttta |
| 9101 | tttcaggcat ttgtaaatgc ccattaataa agtatggttc ataaatttta ccttgtaagt |
| 9161 | gcctaagaaa tgagactaca agctccattt cagcaggaca caataaatat tattttataa |
| 9221 | tgcatctaaa aaaaaaaaaa aaaaa |

SEQ ID NO: 205 Human CD84 Isoform 2 Amino Acid Sequence (NP_003865.1)

| | |
|---|---|
| 1 | maqhhlwill lclqtwpeaa gkdseiftvn gilgesvtfp vniqeprqvk iiawtsktsv |
| 61 | ayvtpgdset apvvtvthrn yyerihalgp nynlvisdlr medagdykad intqadpytt |
| 121 | tkrynlqiyr rlgkpkitqs lmasvnstcn vtltcsveke eknvtynwsp lgeeghvlqi |
| 161 | fqtpedqelt ytctaqnpvs nnsdsisarq lcadiamgfr thhtgllsvl amffllvlil |
| 241 | ssvflfrlfk rrqdaaskkt iytvimasrn tqpaesriyd eilqskvlps keepvntvys |
| 301 | evqfadkmgk astqdskppg tssyeivi |

SEQ ID NO: 206 Human CD84 Transcript Variant 3 cDNA Sequence
(NM_001184881.1; CDS: 80-898)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctggccg gaagcagctg gaaaagactc agaaatcttc acagtgaatg ggattctagg |
| 181 | agagtcagtc actttcccta taaatatcca agaaccacgg caagttaaaa tcattgcttg |
| 241 | gacttctaaa acatctattg cttatgtaac accaggagac tcagaaacag cacccgtagt |
| 301 | tactgtgacc cacagaaatt attatgaacg gatacatgcc ttaggtccga actacaatct |
| 361 | ggtcattagc gatctgagga tggaagacgc aggagactac aaagcagaca taaatacaca |
| 421 | ggctgatccc tacaccacca ccaagcgcta caacctgcaa atctatcgtc ggcttgggaa |
| 481 | accaaaaatt acacaaagtt taatggcatc tgtgaacagc acctataatg tcacactgac |
| 541 | atgctctgta gagaaagaag aaaagaatgt gacatacaat tggagtcccc tgggagaaga |
| 601 | gagtaatatc cttcaaatct tccagactcc tgaggaccaa gagctgactt acacgtgtac |
| 661 | agcccagaac cctgtcagca acaattctga ctccatctct gcccggcagc tctgtgcaga |
| 721 | catcgcaatg ggcttccgta ctcaccacac caggttgctg agcgtgctgg ctatattctt |
| 781 | tctgcttgtt ctcattctgt cttcagtgtt tttattccgt ttgttcaaga gaagacaagg |
| 841 | tgcttccctc caaggaagag ccagtgaaca cagtttattc cgaagtgcag tttgctgata |
| 901 | agatggggaa agccagcaca caggacagta aacctcctgg gacttcaagc tatgaaattg |
| 961 | tgatctaggc tgctaggctg aattctccct ctggaaactg agttacaacc accaatactg |
| 1021 | gcaggttccc tggatccaga tcttctctgc ccaactctta ctaggagatt gcaaactgcc |
| 1081 | acatctcagc ctgtaagcaa agcaggaaac cttctgctga gcatagcttg tgcctaaatg |
| 1141 | gacaaatgga tgcataccct tcctgaaatg actcccttct gaatgaatga caaagcaggt |
| 1201 | tacctagtat agttttccca aacttcttcc catcatagca catgtagaaa ataatatttt |
| 1261 | tatggcacac taggataaac aagcaagatt gctcacttct ggaagctgca tatgactaga |
| 1321 | ggcctcttgt gactggaggt aacaaccctg cccagtaact gtgggagaag gggatcaata |
| 1381 | ttttgcacac ctgtaatagg ccatggcaca ccagccaaga tgctctgctc acagtcagta |
| 1441 | tgtgtgaaga tccctggtgc gtggccttca ccacgcatct tgagcaaatt aggaaaatgt |
| 1501 | acccttcgct tgaggcagat gcagcccttc ccccgagtgc atggcttgga gagcagaatg |
| 1561 | tgggctgcat ataagcacac tcatcccttt gtctgagaat ctttgtgcag ggcataacag |
| 1621 | gcttagtaag tccaaacaca gatgacagtg ctgtgtggat ctctatcaga gttgtggctc |
| 1681 | tcagccatgt agacacactc tccaaatgga gtgttggaaa atattctttc tgcagggtct |
| 1741 | agagactgct gggacacttt tcttggagtg ctacttcaga agccttatag gattttcttt |
| 1801 | ctggccaaga tttccttctg tatcactcca agcagcctca gcagaagaag cagccatgcc |
| 1861 | cagtattccc actctccaaa aggaactgac cagcttatat ttctcacact tctgaggaac |
| 1921 | tgagtataat ccaaccatca aaatagaaga ccttgcaaga aacagagtca ttctccagaa |
| 1981 | ggaacttggg agatgatggt gcagatgatg aaactggatt catcccagtt ccaaagactc |
| 2041 | agagaactag agtttaagct gaggcagagt gccgccaccc tggcatgccc cacaaacaga |
| 2101 | tcaccagcca gcttacacag gcattaactc tcctcaatga ggaagaatca ttcacaactg |
| 2161 | agcaagacat tcatatgatc atttaaggaa gtgtttccct tatgtattag caagtataat |
| 2221 | cggctaactc ctaaatccca ataaatagtc ctaggctaaa cagcaatggg ctacaattag |
| 2281 | gcagataaag acatcagtcc cagtaaatga atccatagac tcatctagca ccaactacca |
| 2341 | ttagcactat gttaggagct gcaaggcccc aaagtagaag atgtgcataa tatctgctct |
| 2401 | tgtgtagctc aggagacaat tccagcacag acactacagt taacgctgaa ctgcagctgc |
| 2461 | aagtaatagc atgaacagtc agaaaaatac cttatgaggg ggcagggcta aagctaggcc |
| 2521 | ttgaaggatg gatgaaattt ggatagagaa tgaagaagac aaagggcctc caagtgagag |
| 2581 | aagcatgaaa aatgagcagg ggcctggatc agtggggtgt attcagagca cctctccaga |
| 2641 | tgcaccatgc atgctcacag tcccttgcct atgtgtggca gagtgtccca gccagatgtg |
| 2701 | tgccctcacc ccatgtccat ttacatgtcc ttcaatgccc acctcaaaag gtacctcttc |
| 2761 | tgtaaagctt tccgtggtat caagaatcaa aattaatcag ggatcttttc acactgctgt |
| 2821 | tttttcctct ttgatccttc tatcactaaa actcatctca ttcagcctta cagcataact |
| 2881 | aattatttgt tttcctcact acattgtaca tgtgggaatt acagataaac ggaagccggc |
| 2941 | tagggtgatg gctcacgcct gtaatcccaa cactttgaga ggccaaggca ggcggatcac |
| 3001 | ctgaggtcag gagttcgaga ttagtctggc caacatggtg aaaccccatc tctactaaaa |
| 3061 | atacaaaatt aaccaggtgt ggtggcacac atctgtagtc ccagctactc tggaggctga |
| 3121 | gacaggagaa tcacttgaac ccaggaagtg gaggttgcag tgagctgaga tcacaccact |
| 3181 | gcactccagc ctgggagaga cagagtgaga ctccatctcg aaaaaaaaaa aaagatagaa |
| 3241 | gccaataagc atggtgcaat caaattctgg caagcattaa atatcaggat gcagctgggc |
| 3301 | acggtggctc acgcctgtaa tcccagcact ttgggaggcc aaggtgggcg gatcacttga |
| 3361 | ggtcaggaat ttgagaggat cctggccagc atggcaaaac cccatctgta cttaaaatac |
| 3421 | aaaaaaatta gctgggcgtg gtggtgcaca cctgtaatcc cagctacttg ggaggctgag |
| 3481 | gtaggagaat tgcttgaacc taggaggtgg aggttgcagt gagctgagat cctgccactg |
| 3541 | cactccaggc tgggcaacag agtgagacca tgtctcaaaa aataaaaata aaataaaata |
| 3601 | atatcaggat gcatacatca gaggctattc ctaatgtaaa ggcactttaa agggagaaga |
| 3661 | ctttcagagt taggcagacc aactaaaagg tcaactgaag cacctaacca gttgtaagga |
| 3721 | cgtgaaagac agcaccccaa gaagagacgt gcaggaagga ggaaagaggc ttggtcataa |
| 3781 | aggatggagg aattccaaag tgacactgaa caggctgcgt ttatcctaaa ataaaaccac |
| 3841 | tcctcactct gtggatgcgt tgaagactca ttcccaaaca tctttattct ctaacttgcc |
| 3901 | ctcttcctct tcctaatatg ctcactcaag taaaattact agtgtcctaa tgcccctatg |
| 3961 | catattgtca aaaataaaaa tcagaagcag gttagatctg ttaggtcttc cagaagagca |
| 4021 | aacctgggat gaagccagag cccaggaatt ctgaaggtag cctttggact caggacaccc |
| 4081 | tactcttgtc tctcctctca gtttctctgc tatgaatctc ctgattcatg aacacgttat |
| 4141 | ctgttcaccc ttctctctag gtcttagttc ttagattttc cttctgtaaa atgcatgtga |
| 4201 | tcttattttc ccctccacaa ctttccagat gaactagact gtgaccaaga ggtctataaa |
| 4261 | atcaaagcat catggaacag gatcttgtat cagaccaaag tgtgccagtt tttaaaaatg |
| 4321 | tgcatcaaaa tggaagtctc agagacagag ccctctggtg gaaagttcta gtaggttagg |
| 4381 | acagtcctgc ctgcagacac cttgggcttt actgagggac tcaactgaga aaatgaggaa |
| 4441 | tgttgcagct catgattctt agaagaagaa agtgaagctt gtttaaaata tgatttaaaa |
| 4501 | aatctgtaga acactgtaaa ctacacaggc tatgagggaa tagcctggtt gggccagctt |
| 4561 | ggaaatcggg cacaggcagg aaggggcctg tctggtttgg gccgtgtcca cagagagcac |
| 4621 | ttcttaggtc ctgcctggag agaaggaatg gctgggctat attttcttcc agactcatta |
| 4681 | tttttcttct gtttgacttt tctctgaatt tcccttgatt tgtataaatt ttctcaataa |
| 4741 | ttagtgacag tgtctactga ttgtaaaatg aagcttgaag gccaggcgca gtggctcatg |
| 4801 | cctgtaatcc tagaattttg ggaggccaag gtgggtggat cacaaggagt tcgagaccag |
| 4861 | cctggccaag gttgtgaaac cgcgtctcta ctaaaaatac aaaaaaatta gcccggcatg |
| 4921 | gtggcacgtg cctgtagtcc cagctactca ggaagctgag gcaacagaat cacttgaacc |
| 4981 | tgggaggtgg aggttgcagt gagccgagat cacgccactg cactccagcc tgggcgagag |
| 5041 | agtgagactc cgtctcaaaa aaaaaaaaaa aaaaaaaagt ttgaagaaca aagacaataa |
| 5101 | gaggaaatat aatgagtggt cataaatgtg ggctctgaca gtagagtgcc tgggtctgca |
| 5161 | tcctggtttc ttagtcatgt gaccttaggc aagttacttt aacctcgctg tacctcaggt |
| 5221 | tgtccatctg taaaatgggg ataataatag tgcctacctt ttaaggttga tgtggggatt |
| 5281 | aaatgaggtg ttgctcatac aggaatgtgc ctgtgcatgg caaagttcgg gaaatttttt |
| 5341 | ataagctgtt ctaggcctga aatcttcaga agatgctaat ctaaattcat gaaataagct |
| 5401 | tcttacaaca gaaatgctgc tagtattatg caaaattaat gttgtatatc aaacttttaa |
| 5461 | ctctcatccc tccttattca gatatatttt gttataagca atgtttgttc ccctcgttat |
| 5521 | tataccacag tctacttacc tgatgctata tctgcctccc cagttagact gagagaacag |
| 5581 | gggatatacc taaataataa taataataat aataataata aataataatg gagagctcct |
| 5641 | tgaagatagg gagcctgtaa gaatcattga gggcttattt tgtataccaa ctgctaaact |
| 5701 | agatgcttca tacattgttg tcaatactca tgacagcctt gtaaagtaga aattaattct |
| 5761 | tccagttaac actaaggctg acatatgaat accttggcaa atctggaaag ctgggaagac |
| 5821 | agtatttgaa ttcaagactt cttgtcacca agggccatgc acttgtactc tgccatgtgg |
| 5881 | ccctttttta cctcctgtgg attctcccta cctggtactt ggccttaggt gtacacacac |
| 5941 | ctggcacttt gcttgacaca taataggtgg accacaaata tctactaaat gaatatttgc |
| 6001 | atatagtaat attttaaggt actaaaagca gctcaaagta aatatattaa tatattaatt |
| 6061 | ccattgctat ctggataacc actcaacttt cctgctgaaa atgcccattt aattaaagaa |
| 6121 | ggttggatag agctctctat atgcattttg gacaggcagg ggtttcaggt cataaacatt |
| 6181 | ctgatgagtt aatataaaat aagagaaact gtaaatttcc actactaaaa atcacaaaaa |
| 6241 | taacagaaac aaaagaagag ataagaattt ggggaattgt gctgaacaat ttagtggtta |
| 6301 | aaaaaaacaa ctgtgcatgt ttagacttaa ataagccccc atccaagtgt gaggggtcca |
| 6361 | gtaatttttc aaaacatatg aaagtgttaa tacatttcga caaaggacca ttaaaaaagt |
| 6421 | cctgaattct gacttgaggg aggaaagtaa tgactaatac attctctaga gacttgcaga |
| 6481 | ctttgggaat tcataaagga atggatgata attattaact gttgctggct gattgcccag |
| 6541 | acagttctca acagccctgt acaagtctct cggtttggga tggatcaatt ctgagactgg |
| 6601 | aaaatggcca aatctttgca aatgagaaat atttttctta taagttctta ttgtaggcaa |
| 6661 | ataattacat agattattca tcagagaatt tttaaatgct cataatctca actctttcat |
| 6721 | ttacaacttg tatttccaat agtttatggg tcatctctgc atagatgtca gaagtcacct |
| 6781 | caagtttagc gtgtccaaaa tctaactcac aggtctgttt ctgacctccc aacttgcttt |
| 6841 | ccttgtgttt ttcctatgct aatgatccac cataatcaaa ataattaaca tttatccagt |
| 6901 | gcctactatg tactattccc tgtcctgttt tacatttact catttaaagt ccataagaaa |
| 6961 | cattaaatct catctgcctt ctgaagaaga tacaaccatg ctctctttta caaagtagga |
| 7021 | aactgggtca cagaaaggtg aagtctttaa ggctgaatca cagtagctca tcctagtaaa |
| 7081 | tagaaaagcc aggattcaac tccaggggct gggtgcagaa ctgctattct tcactgcttc |
| 7141 | accaatcagc agctacccaa ggcagaaaac tttttcatcc ttggctcctt cattctccct |
| 7201 | gtcaccccag atcccctcta catctagtca gagaataggt cctgtcaatt ccaacttctc |
| 7261 | tatatggctc ctctcaggca tgtgccctta attggcctaa ttctctaata caccttccct |
| 7321 | ctacatgctc actccctcag atcattgctt tatcacgtgt tacctgggtt gctattacat |
| 7381 | aaagagcaat ctttctaaaa tgaggatctt atcacttcac ttccacacta aaatgttttt |
| 7441 | cctggggaac cacactcctt agcaatctga cccatcagac cttccaggct gtctcctgcc |
| 7501 | tgctccctaa ggctccagcc acacagaatt atcatgggcc cacacaccca ccaaatcctc |
| 7561 | ccatgccttt gcccatgttg tctgggatgc ccttctctcc cttctgtcta catcaagcat |
| 7621 | cagactgaat atccctcttg tgcggccttc taaaacctcc cgtccaaagc gaaatatatt |
| 7681 | gccctctatt tatactttta cagcatttgg cacacaagta cagagtagta gctttttatc |
| 7741 | acattctctg ataattatat agatatggta tttcttagct ctctctccaa ctggctaata |
| 7801 | agttgctttt tgtctgagtg cctaattttg tgttttgtgt ctgagtgcct cagttcctca |
| 7861 | aaaaaaggtt ttttgattag ttcattattc atttgaacat ggaaattatg ctcactagtg |
| 7921 | gcaaatgcca ctaaccgtat tccagaagct aggtgtcatg tttgcaataa gatatattat |
| 7981 | cccttctaca agtcaccttt tattacaggc atttgtaaat gcccattaat aaagtatggt |
| 8041 | tcataaattt taccttgtaa gtgcctaaga aatgagacta caagctccat ttcagcagga |
| 8101 | cacaataaat attattttat aatgcatcta aaaaaaaaaa aaaaaaa |

SEQ ID NO: 207 Human CD84 Isoform 3 Amino Acid Sequence (NP_001171810.1)

| | |
|---|---|
| 1 | maqhhlwill lclqtwpeaa gkdseiftvn gilgesvtfp vniqeprqvk iiawtsktsv |
| 61 | ayvtpgdset apvvtvthrn yyerihalgp nynlvisdlr medagdykad intqadpytt |
| 121 | tkrynlqiyr rlgkpkitqs lmasvnstcn vtltcsveke eknvtynwsp lgeegnvlqi |
| 181 | fqtpedqelt ytctaqnpvs nhsdsisarq lcadiamgfr thhtgllsvl amffllvlil |
| 241 | ssvflfrlfk rrqgaslqgr asehslfrsa vc |

SEQ ID NO: 208 Human CD84 Transcript Variant 4 cDNA Sequence
(NM_001184882.1; CDS: 80-724)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctgtcgg cttgggaaac caaaaattac acagagttta atggcatctg tgaacagcac |
| 181 | ctgtaatgtc acactgacat gctctgtaga gaaagaagaa aagaatgtga catacaattg |
| 241 | gagtcccctg ggaccagagg gtaatgtcct tcaaatcttc cagactcctg aggaccaaga |
| 301 | gctgacttac acgtgtacag cccagaaccc tgtcagcaac aattctgact ccatctctgc |
| 361 | ccggcagctc tgtgcagaca tcgcaatggg cttccgtact caccacaccg ggttgctgag |
| 421 | cgtgctggct atgttctttc tgcttgttct cattctgtct tcagtgtttt tgttccgttt |
| 481 | gttcaagaga agacaagatg ctgcctcaaa gaaaaccata tacacatata tcatggcttc |
| 541 | aaggaacacc cagccagcag agtccagaat ctatgatgaa atcctgcagt ccaaggtgct |
| 601 | tccctccaag gaagagccag tgaacacagt ttattccgaa gtgcagtttg ctgataagat |
| 661 | ggggaaagcc agcacacagg acagtaaacc tcctgggact tcaagctatg aaattgtgat |
| 721 | ctaggctgct gggctgaatt ctccctctgg aaactgagtt acaaccacca atactggcag |
| 781 | gttccctgga tccagatctt ctctgcccaa ctcttactgg gagattgcaa actgccacat |
| 841 | ctcagcctgt aagcaaagca ggaaaccttc tgctgggcat agcttgtgcc taaatggaca |
| 901 | aatggatgca tacccttcct gaaatgactc ccttctgaat gaatgacaaa gcaggttacc |
| 961 | tagtatagtt ttcccaaact tcttcccatc atagcacatg tagaaaataa tatttttatg |
| 1021 | gcacactggg ataaacaagc aagattgctc acttctggaa gctgcatatg actagaggcc |
| 1081 | tcttgtgact ggaggtaaca accctgccca gtaactgtgg gagaagggga tcaatatttt |
| 1141 | gcacacctgt aataggccat ggcacaccag ccaagatgct ctgctcacag tcagtatgtg |
| 1201 | tgaagatccc tggtgcgtgg ccttcaccac gcatcttgag caaattagga aaatgtaccc |
| 1261 | ttcgcttgag gcagatgcag cccttccccc gagtgcatgg cttggagagc agaatgtggg |
| 1321 | ctgcatataa gcacactcat ccctttgtct gggaatcttt gtgcagggca taacaggctt |
| 1381 | agtaagtcca aacacagatg acagtgctgt gtgggtctct gtcagagttg tggctctcag |
| 1441 | ccatgtagac acactctcca aatggagtgt tggaaaatgt tctttctgca gggtctagag |
| 1501 | actgctggga cacttttctt ggagtgctac ttcagaagcc ttataggatt ttctttctgg |
| 1561 | ccaagatttc cttctgtatc actccaagca gcctcagcag aagaagcagc catgcccagt |
| 1621 | attcccactc tccaaaagga actgaccagc ttatatttct cacacttctg gggaactggg |
| 1681 | tataatccaa ccatcaaaat agaagacctt gcaagaagca gagtcattct ccagaaggaa |
| 1741 | cttgggagat gatggtgcag atgatgaaac tgggttcatc ccagttccaa agactcagag |
| 1801 | aactagagtt taagctgagg cagagtgccg ccaccctggc atgccccaca aacagatcac |
| 1861 | cagccagctt acacaggcat taactctcct caatgaggaa gaatcattca caactgagca |
| 1921 | agacattcat atgatcattt aaggaagtgt ttcccttatg tgttagcaag tataatcggc |
| 1981 | taactcctaa atcccaatga atagtcctag gctggacagc aatgggctgc aattaggcag |
| 2041 | ataaagacat cagtcccagt aaatgaatcc atagactcat ctagcaccaa ctaccattag |
| 2101 | cactatgtta ggagctgcaa ggccccaaag tagaagatgt gcataatgtc tgctcttgtg |
| 2161 | tagctcagga gacaattcca gcacagacac tacagttaac gctgaactgc agctgcaagt |
| 2221 | aatagcatga acagtcagaa aaatacctta tgagggggca gggctgaagc tgggccttga |
| 2281 | aggatggatg aaatttggat agagaatgag gaagacagag ggcctccaag tgagagaagc |
| 2341 | atgaaaaatg agcaggggcc tggatcagtg gggtgtattc agagcacctc tccagatgca |
| 2401 | ccatgcatgc tcacagtccc ttgcctatgt gtggcagagt gtcccagcca cctgtgtgcc |
| 2461 | ctcaccccat gtccatttac atgtccttca atgcccacct caaaaggtac ctcttctgta |
| 2521 | aagctttccc tggtatcagg aatcaaaatt aatcagggat cttttcacac tgctgttttt |
| 2581 | tcctctttgg tccttctatc actaaaactc atctcattca gccttacagc ataactaatt |
| 2641 | atttgttttc ctcactacat tgtacatgtg ggaattacag ataaacggaa gccggctggg |
| 2701 | gtggtggctc acgcctgtaa tcccaacact ttgggaggcc aaggcaggcg gatcacctga |
| 2761 | ggtcaggagt tcgagattag tctggccaac atggtgaaac cccatctcta ctaaaaatac |
| 2821 | gaaattagcc aggtgtggtg gcacacatct gtagtcccag ctactctgga ggctgagaca |
| 2881 | ggagaatcgc ttgaacccag gaagtggagg ttgcagtgag ctgagatcac accactgcac |
| 2941 | tccagcctgg gagagacaga gtgagactcc atctcgaaaa aaaaaaaaag atagaagcca |
| 3001 | ataagcatgg tgcaatcaaa ttctggcaag cattaaatat caggatgcag ctgggcacgg |
| 3061 | tggctcacgc ctgtaatccc agcactttgg gaggccaagg tgggcggatc acttgaggtc |
| 3121 | aggaatttga gaggatcctg gccagcatgg caaaacccca tctgtactta aaatacaaaa |
| 3181 | aaattagctg ggcgtggtgg tgcacacctg taatcccagc tacttgggag gctgaggtgg |
| 3241 | gagaattgct tgaacctggg aggtggaggt tgcagtgagc tgagatcctg ccactgcact |
| 3301 | ccaggctggg caacagagtg agaccatgtc tcaaaaaata aaaataaaat aaaataatat |
| 3361 | caggatgcat acatcagagg ctgttcctag tgtaaaggca ctttggaggg agaagacttt |
| 3421 | cagagttagg cagaccaact aagaggtcag ctgaagcacc taaccagttg taaggaggtg |
| 3481 | aaagacagca ccccaagaag agacgtgcag gaaggaggaa agaggcttgg tcataaagga |
| 3541 | tggaggaatt ccaaagtgac actgaacagg ctgcgtttat cctaaaataa aaccactcct |
| 3601 | cactctgtgg atgcgttgaa gactcattcc caaacatctt tattctctaa cttgccctct |
| 3661 | tcctcttcct aatatgctca ctcaagtaaa attactagtg tcctaatgcc cctatgcata |
| 3721 | ttgtcaaaaa taaaaatcag aagcaggtta gatctgttag gtcttccaga agagcaaacc |
| 3781 | tgggatgaag ccagagccca ggaattctga aggtagcctt tggactcagg acaccctact |
| 3841 | cttgtctctc ctctcagttt ctctgctatg aatctcctga ttcatgaaca cgttatctgt |
| 3901 | tcacccttct ctctaggtct tagttcttag attttccttc tgtaaaatgc atgtgatctt |
| 3961 | attttcccct ccacaacttt ccagatgaac tagactgtga ccaagaggtc tataaaatca |
| 4021 | aagcatcatg gaacaggatc ttgtatcaga ccaaagtgtg ccagttttta aaaatgtgca |
| 4081 | tcaaaatgga agtctcagag acagagccct ctggtggaaa gttctagtag gttaggacag |
| 4141 | tcctgcctgc agacaccttg ggctttactg agggactcaa ctgagaaaat gaggaatgtt |
| 4201 | gcagctcatg attcttagaa ga.agaaagt aagcttgttt aaaatatgat ttaaaaaatc |
| 4261 | tgtagaacac tgtaaactac acaggctatg agggaatagc ctggttgggc cagcttggaa |
| 4321 | atcgggcaca ggcaggaagg ggcctgtctg gtttgggccg tgtccacaga gagcacttct |
| 4381 | taggtcctgc ctggagagaa ggaatggctg ggctatattt tcttccagac tcattatttt |
| 4441 | tcttctgttt gacttttctc tgaatttccc ttgatttgta taaattttct caataattag |
| 4501 | tgacagtgtc tactgattgt aaaatgaagc ttgaaggcca ggcgcagtgg ctcatgcctg |
| 4561 | taatcctaga attttgggag gccaaggtgg gtggatcaca aggagttcga gaccagcctg |
| 4621 | gccaaggttg tgaaaccgcg tctctactaa aaatacaaaa aaattagccg ggcatggtgg |
| 4681 | cacgtgcctg tagtcccagc tactcaggaa gctgaggcaa cagaatcact tgaacctggg |
| 4741 | aggtggaggt tgcagtgagc cgagatcacg ccactgcact ccagcctggg cgagagagtg |
| 4801 | agactccgtc tcaaaaaaaa aaaaaaaaaa aaaagtttga agaacaaaga caataagagg |
| 4861 | aaatataatg agtggtcata aatgtgggct ctgacagtag agtgcctggg tctgcatcct |
| 4921 | ggtttcttag tcatgtgacc ttaggcaagt tactttaacc tcgctgtacc tcaggttgtc |
| 4981 | catctgtaaa atggggataa taatagtgcc taccttttaa ggttgatgtg gggattaaat |
| 5041 | gaggtgttgc tcatacagga atgtgcctgt gcatggcaaa gttcgggaaa ttttttataa |
| 5101 | gctgttctag gcctgaaatc ttcagaagat gctaatctaa attcatgaaa taagcttctt |
| 5161 | acaacagaaa tgctgctagt attatgcaaa attaatgttg tatatcaaac ttttaactct |
| 5221 | catccctcct tattcagata tattttgtta taagcaatgt ttgttcccct cgttattata |
| 5281 | ccacagtcta cttacctgat gctatatctg cctccccagt tagactgaga gaacagggga |
| 5341 | tatacctaaa taataataat aataataata ataataaata ataatggaga gctccttgaa |
| 5401 | gatagggagc ctgtaagaat cattgagggc ttattttgta taccaactgc taaactagat |
| 5461 | gcttcataca ttgttgtcaa tactcatgac agccttgtaa agtagaaatt aattcttcca |
| 5521 | gttaacacta aggctgacat atgaatacct tggcaaatct ggaaagctgg gaagacagta |
| 5581 | tttgaattca agacttcttg tcaccaaggg ccatgcactt gtactctgcc atgtggccct |
| 5641 | tttttacctc ctgtggattc tccctacctg gtacttggcc ttaggtgtac acacacctgg |
| 5701 | cactttgctt gacacataat aggtggacca caaatatcta ctaaatgaat atttgcatat |
| 5761 | agtaatattt taaggtacta aaagcagctc aaagtaaata tattaatata ttaattccat |
| 5821 | tgctatctgg ataaccactc aactttcctg ctgaaaatgc ccatttaatt aaagaaggtt |
| 5881 | ggatagagct ctctatatgc attttggaca ggcaggggtt tcaggtcata aacattctga |
| 5941 | tgagttaata taaaataaga gaaactgtaa atttccacta ctaaaaatca caaaaataac |
| 6001 | agaaacaaaa gaagagataa gaatttgggg aattgtgctg aacaatttag tggttaaaaa |
| 6061 | aaacaactgt gcatgtttag acttaaataa gcccccatcc aagtgtgagg ggtccagtaa |
| 6121 | tttttcaaaa catatgaaag tgttaataca tttcgacaaa ggaccattaa aaaagtcctg |
| 6181 | aattctgact tgagggagga aagtaatgac taatacattc tctagagact tgcagacttt |
| 6241 | gggaattcat aaaggaatgg atgataatta ttaactgttg ctggctgatt gcccagacag |
| 6301 | ttctcaacag ccctgtacaa gtactctggg ttgggatgga tcaattctga gactggaaaa |
| 6361 | tggccaaatc tttgcaaatg agaaatattt ttcttataag ttcttattgt aggcaaataa |
| 6421 | ttacatagat tattcatcag agaattttta aatgctcata atctcaactc tttcatttac |
| 6481 | aacttgtatt tccaatagtt tatgggtcat ctctgcatag atgtcagaaa tcacctcaag |
| 6541 | tttagcgtgt ccaaaatcta actcacaggt ctgtttctga cctcccaact tgctttcctt |
| 6601 | gtgtttttcc tatgctaatg atccaccata atcaaaataa ttaacattta tccagtgcct |
| 6661 | actatgtact attccctgtc ctgttttaca tttactcatt taaagtccat aagaaacatt |
| 6721 | aaatctcatc tgccttctga agaagataca accatgctct cttttacaaa gtaggaaact |
| 6781 | gggtcacaga aaggtgaagt ctttaaggct gaatcacagt agctcatcct agtaaataga |
| 6841 | aaagccagga ttcaactcaa ggggctgggt gcagaactgc tattcttcac tgcttcacca |
| 6901 | atcagcagct acccaaggca gaaaactttt tcatccttgg ctccttcatt ctccctgtca |
| 6961 | ccccagatcc cctctacatc tagtcagaga ataggtcctg tcaattccaa cttctctata |
| 7021 | tggctcctct caggcatgtg cccttaattg gcctaattct ctaatacacc ttccctctac |
| 7081 | atgctcactc cctcagatca ttgctttatc acgtgttacc tgggttgcta ttacataaag |
| 7141 | agcaatcttt ctaaaatgag gatcttatca cttcacttcc acactaaaat gtttttcctg |
| 7201 | gggaaccaca ctccttagca atctgaccca tcagaccttc caggctgtct cctgcctgct |
| 7261 | ccctaaggct ccagccacac agaattatca tgggcccaca cacccaccaa atcctcccat |
| 7321 | gcctttgccc atgttgtctg ggatgccctt ctctcccttc tgtctacatc aagcatcaga |
| 7381 | ctgaatatcc ctcttgtgcg gccttctaaa acctcccgtc caaagcgaaa tatattgccc |
| 7441 | tctatttata cttttacagc atttggcaca caagtacaga gtagtagctt tttatcacat |
| 7501 | tctctgataa ttatatagat atggtatttc ttagctctct ctccaactgg ctaataagtt |
| 7561 | gctttttgtc tgagtgccta attttgtgtt ttgtgtctga gtgcctcagt tcctcaaaaa |
| 7621 | aaggtttttt gattagttca ttattcattt gaacatggaa attatgctca ctagtggcaa |
| 7681 | atgccactaa ccgtattcca gaagctaggt gtcatgtttg caataagata tattatccct |
| 7741 | tctacaagtc accttttatt tcaggcattt gtaaatgccc attaataaag tatggttcat |
| 7801 | aaattttacc ttgtaagtgc ctaagaaatg agactacaag ctccatttca gcaggacaca |
| 7861 | ataaatatta ttttataatg catctaaaaa aaaaaaaaaa aaa |

SEQ ID NO: 209 Human CD84 Isoform 4 Amino Acid Sequence (NP_001171811.1)

| | |
|---|---|
| 1 | maqhhlwill lclqtcrlgk pkitqslmas vnstcnvtlt csvekeeknv tynwsplgee |
| 61 | gnvlqifqtp edqeltytct aqnpvsnnsd sisarqlcad iamgfrthht gllsvlamff |
| 121 | llvlilssvf lfrlfkrrqd aaskktiyty imasrntqpa esriydeilq skvlpskeep |
| 181 | vntvysevqf adkmgkastq dskppgtssy eivi |

SEQ ID NO: 210 Human CD84 Transcript Variant 5 cDNA Sequence
(NM_001330742.1; CDS: 80-1099)

| | |
|---|---|
| 1 | ggaggaagaa aactcaagtg aaactgactc tgctagaaca gtgccgtgct tttccacaga |
| 61 | aggttagacc ctgaaagaga tggctcagca ccacctatgg atcttgctcc tttgcctgca |
| 121 | aacctggccg gaagcagctg gaaaagactc agaaatcttc acagtgaatg ggattctggg |
| 181 | agagtcagtc actttccctg taaatatcca agaaccacgg caagttaaaa tcattgcttg |
| 241 | gacttctaaa acatctgttg cttatgtaac accaggagac tcagaaacag cacccgtagt |
| 301 | tactgtgacc cacagaaatt attatgaacg gatacatgcc ttaggtccga actacaatct |
| 361 | ggtcattagc gatctgagga tggaagacgc aggagactac aaagcagaca taaatacaca |
| 421 | ggctgatccc tacaccacca ccaagcgcta caacctgcaa atctatcgtc ggcttgggaa |
| 481 | accaaaaatt acacagagtt taatggcatc tgtgaacagc acctgtaatg tcacactgac |
| 541 | atgctctgta gagaaagaag aaaagaatgt gacatacaat tggagtcccc tgggagaaga |
| 601 | gggtaatgtc cttcaaatct tccagactcc tgaggaccaa gagctgactt acacgtgtac |
| 661 | agcccagaac cctgtcagca acaattctga ctccatctct gcccggcagc tctgtgcaga |
| 721 | catcgcaatg ggcttccgta ctcaccacac cgggttgctg agcgttctgg ctatgttctt |
| 781 | tctgcttgtt ctcattctgt cttcagtgtt tttgttccgt ttgttcaaga gaagacaagg |
| 841 | ttcctgcttg aacaccttca ctaagaaccc ttatgctgcc tcaaagaaaa ccatatacac |
| 901 | atatatcatg gcttcaagga acacccagcc agcagagtcc agaatctatg atgaaatcct |
| 961 | gcagtccaag gtgcttccct ccaaggaaga gccagtgaac acagtttatt ccgaagtgca |
| 1021 | gtttgctgat aagatgggga aagccagcac acaggacagt aaacctcctg ggacttcaag |
| 1081 | ctatgaaatt gtgatctagg ctgctgggct gaattctccc tctggaaact gagttacaac |
| 1141 | caccaatact ggcaggttcc ctggatccag atcttctctg cccaactctt actgggagat |
| 1201 | tgcaaactgc cacatctcag cctgtaagca aagcaggaaa ccttctgctg ggcatagctt |
| 1261 | gtgcctaaat ggacaaatgg atgcataccc ttcctgaaat gactcccttc tgaatgaatg |
| 1321 | acaaaggagg ttacctagta tagttttccc aaacttcttc ccatcatagc acatgtagaa |
| 1381 | aataatattt ttatggcaca ctgggataaa caagcaagat tgctcacttc tggaagctgc |
| 1441 | atatgactag aggcctcttg tgactggagg taacaaccct gcccagtaac tgtgggagaa |
| 1501 | ggggatcaat attttgcaca cctgtaatag gccatggcac accagccaag atgctctgct |
| 1561 | cacagtcagt atgtgtgaag atccctggtg cgtggccttc accacgcatc ttgagcaaat |
| 1621 | taggaaaatg tacccttcgc ttgaggcaga tgcagccctt cccccgagtg catggcttgg |
| 1681 | agagcagaat gtgggctgca tataagcaca ctcatccctt tgtctgggaa tctttgtgca |
| 1741 | gggcataaca ggcttagtaa gtccaaacac agatgacagt gctgtgtggg tctctgtcag |
| 1801 | agttgtggct ctcagccatg tagacacact ctccaaatgg agtgttggaa aatgttcttt |
| 1861 | ctgcagggtc tagagactgc tgggacactt ttcttggagt gctacttcag aagccttata |
| 1921 | ggattttctt tctggccaag atttccttct gtatcactcc aagcagcctc agcagaagaa |
| 1981 | gcagccatgc ccagtattcc cactctccaa aaggaactga ccagcttata tttctcacac |
| 2041 | ttctggggaa ctgggtataa tccaaccatc aaaatagaag accttgcaag aagcagagtc |
| 2101 | attctccaga aggaacttgg gagatgatgg tgcagatgat gaaactgggt tcatcccagt |
| 2161 | tccaaagact cagagaacta gagtttaagc tgaggcagag tgccgccacc ctggcatgcc |
| 2221 | ccacaaacag atcaccagcc agcttacaca ggcattaact ctcctcaatg aggaagaatc |
| 2281 | attcacaact gagcaagaca ttcatatgat catttaagga agtgtttccc ttatgtgtta |
| 2341 | gcaagtataa tcggctaact cctaaatccc aatgaatagt cctaggctgg acagcaatgg |
| 2401 | gctgcaatta ggcagataaa gacatcagtc ccagtaaatg aatccataga ctcatctagc |
| 2461 | accaactacc attagcacta tgttaggagc tgcaaggccc caaagtagaa gatgtgcata |
| 2521 | atgtctgctc ttgtgtagct caggagacaa ttccagcaca gacactacag ttaacgctga |
| 2581 | actgcagctg caagtaatag catgaacagt cagaaaaata ccttatgagg gggcagggct |
| 2641 | gaagctgggc cttgaaggat ggatgaaatt tggatagaga atgaggaaga cagagggcct |
| 2701 | ccaagtgaga gaagcatgaa aaatgagcag gggcctggat cagtggggtg tattcagagc |
| 2761 | acctctccag atgcaccatg catgctcaca gtcccttgcc tatgtgtggc agagtgtccc |
| 2821 | agccagatgt gtgccctcac cccatgtcca tttacatgtc cttcaatgcc cacctcaaaa |
| 2881 | ggtacctctt ctgtaaagct ttccctggta tcaggaatca aaattaatca gggatctttt |
| 2941 | cacactgctg ttttttcctc tttggtcctt ctatcactaa aactcatctc attcagcctt |
| 3001 | acagcataac taattatttg ttttcctcac tacattgtac atgtgggaat tacagataaa |
| 3061 | cggaagccgg ctggggtggt ggctcacgcc tgtaatccca acactttggg aggccaaggc |
| 3121 | aggcggatca cctgaggtca ggagttcgag attagtctgg ccaacatggt gaaaccccat |
| 3181 | ctctactaaa aatacgaaat tagccaggtg tggtggcaca catctgtagt cccagctact |
| 3241 | ctggaggctg agacaggaga atcgcttgaa cccaggaagt ggaggttgca gtgagctgag |
| 3301 | atcacaccac tgcactccag cctgggagag acagagtgag actccatctc gaaaaaaaaa |
| 3361 | aaaagataga agccaataag catggtgcaa tcaaattctg gcaagcatta aatatcagga |
| 3421 | tgcagctggg cacggtggct cacgcctgta atcccagcac tttgggaggc caaggtgggc |
| 3481 | ggatcacttg aggtcaggaa tttgagagga tcctggccag catggcaaaa ccccatctgt |
| 3541 | acttaaaata caaaaaaatt agctgggcgt ggtggtgcac acctgtaatc ccagctactt |
| 3601 | gggaggctga ggtgggagaa ttgcttgaac ctgggaggtg gaggttgcag tgagctgaga |
| 3661 | tcctgccact gcactccagg ctgggcaaca gagtgagacc atgtctcaaa aaataaaaat |
| 3721 | aaaataaaat aatatcagga tgcatacatc agaggctgtt cctagtgtaa aggcactttg |
| 3781 | gagggagaag actttcagag ttaggcagac caactaagag gtcagctgaa gcacctaacc |
| 3841 | agttgtaagg aggtgaaaga cagcacccca agaagagacg tgcaggaagg aggaaagagg |
| 3901 | cttggtcata aaggatggag gaattccaaa gtgacactga acaggctgcg tttatcctaa |
| 3961 | aataaaacca ctcctcactc tgtggatgcg ttgaagactc attcccaaac atctttattc |
| 4021 | tctaacttgc cctcttcctc ttcctaatat gctcactcaa gtaaaattac tagtgtccta |
| 4081 | atgcccctat gcatattgtc aaaaataaaa atcagaagca ggttagatct gttaggtctt |
| 4141 | ccagaagagc aaacctggga tgaagccaga gcccaggaat tctgaaggta gcctttggac |
| 4201 | tcaggacacc ctactcttgt ctctcctctc agtttctctg ctatgaatct cctgattcat |
| 4261 | gaacacgtta tctgttcacc cttctctcta ggtcttagtt cttagatttt ccttctgtaa |
| 4321 | aatgcatgtg atcttatttt cccctccaca actttccaga tgaactagac tgtgaccaag |
| 4381 | aggtctataa aatcaaagca tcatggaaca ggatcttgta tcagaccaaa gtgtgccagt |
| 4441 | ttttaaaaat gtgcatcaaa atggaagtct cagagacaga gccctctggt ggaaagttct |
| 4501 | agtaggttag gacagtcctg cctgcagaca ccttgggctt tactgaggga ctcaactgag |
| 4561 | aaaatgagga atgttgcagc tcatgattct tagaagaaga aagtgaagct tgtttaaaat |
| 4621 | atgatttaaa aaatctgtag aacactgtaa actacacagg ctatgaggga atagcctggt |
| 4681 | tgggccagct tggaaatcgg gcacaggcag gaaggggcct gtctggtttg ggccgtgtcc |
| 4741 | acagagagca cttcttaggt cctgcctgga gagaaggaat ggctgggcta tattttcttc |
| 4801 | cagactcatt atttttcttc tgtttgactt ttctctgaat ttcccttgat ttgtataaat |
| 4861 | tttctcaata attagtgaca gtgtctactg attgtaaaat gaagcttgaa ggccaggcgc |
| 4921 | agtggctcat gcctgtaatc ctagaatttt gggaggccaa ggtgggtgga tcacaaggag |
| 4981 | ttcgagacca gcctggccaa ggttgtgaaa ccgcgtctct actaaaaata caaaaaaatt |
| 5041 | agccgggcat ggtggcacgt gcctgtagtc ccagctactc aggaagctga ggcaacagaa |
| 5101 | tcacttgaac ctgggaggtg gaggttgcag tgagccgaga tcacgccact gcactccagc |
| 5161 | ctgggcgaga gagtgagact ccgtctcaaa aaaaaaaaaa aaaaaaaaag tttgaagaac |
| 5221 | aaagacaata agaggaaata taatgagtgg tcataaatgt gggctctgac agtagagtgc |
| 5281 | ctgggtctgc atcctggttt cttagtcatg tgaccttagg caagttactt taacctcgct |
| 5341 | gtacctcagg ttgtccatct gtaaaatggg gataataata gtgcctacct tttaaggttg |
| 5401 | atgtggggat taaatgaggt gttgctcata caggaatgtg cctgtgcatg gcaaagttcg |
| 5461 | ggaaattttt tataagctgt tctaggcctg aaatcttcag aagatgctaa tctaaattca |
| 5521 | tgaaataagc ttcttacaac agaaatgctg ctagtattat gcaaaattaa tgttgtatat |
| 5581 | caaactttta actctcatcc ctccttattc agatatattt tgttataagc aatgtttgtt |
| 5641 | cccctcgtta ttataccaca gtctacttac ctgatgctat atctgcctcc ccagttagac |
| 5701 | tgagagaaca ggggatatac ctaaataata ataataataa taataataat aaataataat |
| 5761 | ggagagctcc ttgaagatag ggagcctgta accatcattg agggcttatt ttgtatacca |
| 5821 | actgctaaac tagatgcttc atacattgtt gtcaatactc atgacagcct tgtaaagtag |
| 5881 | aaattaattc ttccagttaa cactaaggct gacatatgaa taccttggca aatctggaaa |
| 5941 | gctgggaaga cagtatttga attcaagact tcttgtcacc aagggccatg cacttgtact |
| 6001 | ctgccatgtg gccctttttt acctcctgtg gattctccct acctggtact tggccttagg |
| 6061 | tgtacacaca cctggcactt tgcttgacac ataataggtg gaccacaaat atctactaaa |
| 6121 | tgaatatttg catatagtaa tattttaagg tactaaaagc agctcaaagt aaatatatta |
| 6181 | atatattaat tccattgcta tctggataac cactcaactt tcctgctgaa aatgcccatt |
| 6241 | taattaaaga aggttggata gagctctcta tatgcatttt ggacaggcag gggtttcagg |
| 6301 | tcataaacat tctgatgagt taatataaaa taagagaaac tgtaaatttc cactactaaa |
| 6361 | aatcacaaaa ataacagaaa caaaagaaga gataagaatt tggggaattg tgctgaacaa |
| 6421 | tttagtggtt aaaaaaaaca actgtgcatg tttagactta aataagcccc catccaagtg |
| 6481 | tgaggggtcc agtaattttt caaaacatat gaaagtgtta atacatttcg acaaaggacc |
| 6541 | attaaaaaag tcctgaattc tgacttgagg gaggaaagta atgactaata cattctctag |
| 6601 | agacttgcag actttgggaa ttcataaagg aatggatgat aattattaac tgttgctggc |
| 6661 | tgattgccca gacagttctc aacagccctg tacaagtctc tgggtttggg atggatcaat |
| 6721 | tctgagactg gaaaatggcc aaatctttgc aaatgagaaa tatttttctt ataagttctt |
| 6781 | attgtaggca aataattaca tagattattc atcagagaat ttttaaatgc tcataatctc |
| 6841 | aactctttca tttacaactt gtatttccaa tagtttatgg gtcatctctg catagatgtc |
| 6901 | accagtcacc tcaagtttag cgtgtccaaa atctaactca caggtctgtt tctgacctcc |
| 6961 | caacttgctt tccttgtgtt tttcctatgc taatgatcca ccataatcaa aataattaac |
| 7021 | atttatccag tgcctactat gtactattcc ctgtcctgtt ttacatttac tcatttaaag |
| 7081 | tccataagaa acattaaatc tcatctgcct tctgaagaag atacaaccat gctctctttt |
| 7141 | acaaagtagg aaactgggtc acagaaaggt gaagtcttta aggctgaatc acagtagctc |
| 7201 | atcctagtaa atagaaaagc caggattcaa ctccaggggc tgggtgcaga actgctattc |
| 7261 | ttcactgctt caccaatcag cagctaccca aggcagaaaa ctttttcatc cttggctcct |
| 7321 | tcattctccc tgtcacccca gatcccctct acatctagtc agagaatagg tcctgtcaat |
| 7381 | tccaacttct ctatatggct cctctcaggc atgtgccctt aattggccta attctctaat |
| 7441 | acaccttccc tctacatgct cactccctca gatcattgct ttatcacgtg ttacctgggt |
| 7501 | tgctattaca taaagagcaa tctttctaaa atgaggatct tatcacttca cttccacact |
| 7561 | aaaatgtttt tcctggggaa ccacacttct tagcaatctg acccatcaga ccttccaggc |
| 7621 | tgtctcctgc ctgctcccta aggctccagc cacacagaat tatcatgggc ccacacaccc |
| 7681 | accaaatcct cccatgcctt tgcccatgtt gtctgggatg cccttctctc ccttctgtct |
| 7741 | acatcaagca tcagactgaa tatccctctt gtgcggcctt ctaaaacctc ccgtccaaag |
| 7801 | cgaaatatat tgccctctat ttatactttt acagcatttg gcacacaagt acagagtagt |
| 7861 | agctttttat cacattctct gataattata tagatatggt atttcttagc tctctctcca |
| 7921 | actggctaat aagttgcttt ttgtctgagt gcctaatttt gtgttttgtg tctgagtgcc |
| 7981 | tcagttcctc aaaaaaaggt tttttgatta gttcattatt catttgaaca tggaaattat |
| 8041 | gctcactagt ggcaaatgcc actaaccgta ttccagaagc taggtgtcat gtttgcaata |
| 8101 | agatatatta tctcttctac aagtcacctt ttatttcagg catttgtaaa tgcccattaa |
| 8161 | taaagtatgg ttcataaatt ttaccttgta agtgcctaag aaatgagact acaagctcca |
| 8221 | tttcagcagg acacaataaa tattatttta taatgcatct a |

SEQ ID NO: 211 Human CD84 Isoform 5 Amino Acid Sequence (NP_001317671.1)

| | |
|---|---|
| 1 | maqhhlwill lclqtwpeaa gkdseiftvn gilgesvtfp vniqeprqvk iiawtsktsv |
| 61 | ayvtpgdset apvvtvthrn yyerihalgp nynlvisdlr medagdykad intqadpytt |
| 121 | tkrynlqivr rlgkpkitqs lmasvnstcn vtltcsveke eknvtvnwsp lgeegnvlqi |
| 181 | fqtpedqelt ytctaqnpvs nnsdsisarq lcadiamgfr thhtgllsvl amffllvlil |
| 241 | ssvflfrlfk rrqgsclntf tknpyaaskk tiytyimasr ntqpaesriy deilqskylp |
| 301 | skeepvntvy sevqfadkmq kastqdskpp gtssyeivi |

SEQ ID NO: 212 Mouse CD84 Transcript Variant 1 cDNA Sequence (NM_013489.3;
CDS: 180-1169)

| | |
|---|---|
| 1 | agtgcttgga gttcctctgt gactgaccac ttcttccttt tctgtctaat ggtgaacacc |
| 61 | tttctggacc agctctggac cagaatctga tttatgctct gctccggaaa caccacactg |
| 121 | aagtgaaagc agctaccaca ccagttattt ttcctcagaa gactggagtc tgactggaca |
| 181 | tggcccagcg ccatctgtgg atctggttcc tttgcctaca aacctggtct gaagcagcag |
| 241 | gaaaagatgc agacccggtg gtaatgaatg ggattcttgg ggagtcagtt actttcctct |
| 301 | taaatattca agaaccaaag aaaattgaca acattgactg gacttctcaa tcatctgttg |
| 361 | cttttataaa accaggagta aataaagctg aagttaccat aacccagggc acttataaag |
| 421 | gacgaataga aatcatagat cagaagtatg acatggtcat tagagacctg aggatggaag |
| 481 | atgcaggaac ttacaaagca gacatcaatg aagagaatga ggaaaccatc accaagatct |
| 541 | actaccttca tatctaccgt cgacttaaaa caccaaaaat tacacagagt ttgatatcat |
| 601 | ctttgaacaa tacctgtaat atcacactga catgctctgt ggaaaaggaa gaaaaggatg |
| 661 | tcacatatag ctggagtccc tttggagaga aaagcaatgt ccttcaaatc gtccactccc |
| 721 | ccatggacca aaaactgacc tacacatgta cagcccagaa ccctgtcagc aacagttctg |
| 781 | actctgtcac tgtccagcag ccatgtacag acactccaag cttccatcct cgccatgctg |
| 841 | tgttgccagg aggattggcc gtgctctttc tgcttattct cattccgatg ttggcatttc |
| 901 | tgttccgttt gtataagaga aggcgagaca ggattgtcct ggaagcagat gatgtctcaa |
| 961 | agaaaacagt atatgctgta gtttcaagaa atgctcaacc cacagagtcc agaatctatg |
| 1021 | atgaaatccc taagtccaag atgctgtcct gtaagaaaga tccggtgacc accatttatt |
| 1081 | cctaagtgca gctttctgag aagatgaagg aaaccaacat gaaggacaga agtctgccta |
| 1141 | aggatttggg taatgaaatt gttgtctagg tgattctcta agaccacgaa ggacacaagg |
| 1201 | acaagtcatc tatgaggatt gaatcaacgg tttcagtctt ttggatataa cctgggccag |
| 1261 | ccaagggatt taggaatgaa gcaagctccg tgggtagagg tctgatcccc agtgtgtaat |
| 1321 | gttaggggcc atgtacagga ttgactctca ggcccacaga tctttaccca gagaaaccct |
| 1381 | gacctgctcc catgatgttt ttcctgggga aaggacccta gggaactcaa cctttatgca |
| 1441 | atcagacatg cctctcagag actgtctaac agcttccaga ctaatctctg tgcagtactt |
| 1501 | agtcttacaa ctctcacggg caacggcttc aagttccaat tttacgatgt gtctagcctg |
| 1561 | ggatgactgt ttagtttcta atgtggcgag aatgtatgtt accatgtagg aagcacagac |
| 1621 | tatggcaatc tataaatgat ttgtggcatg agactgatgt ccgaatttag gggaagggga |
| 1681 | atggtcttac ttaggcattt tatggaaatt gagtctctct ccccgagaag agggtgatga |
| 1741 | agcagcatcc acgtctgcct cttctccagt aacctgcttg ttatcgacaa tgtccagccg |
| 1801 | atggtaatga actgaaacaa atgcgcttga aagagatgaa tcaatttgag atttaacaaa |
| 1861 | tcgggtcaat ttctgaaatg cccaaggaca gaaggagatc aataatagga gtcccaatag |
| 1921 | gggccctaaa agggagggca acaaggttga aagtcagggg gaggttgaaa accagttttg |
| 1981 | gtagtctacc ttccccctag gccattgtaa atacttgtgt atgggtgtaa ctcagctatc |
| 2041 | tatagttcta agtatccact ctggttcctc tttaggtctt aaacttactt cttcatagtt |
| 2101 | gatggtaaat tactgtgtaa ggcagtggcc tagcttttat taaaagtgat agtgtaatgc |
| 2161 | cagaggctat tatgaatgta actgaatagg caacactctc cctgaattct aagtccatgg |
| 2221 | tctgttcaag ggctttttag gacattggaa caccagtgaa ggcttagcta tgtcagaatt |
| 2281 | caatcttaaa atgcacttat aataagataa tattaaaaga gagcacatgg atctatacac |
| 2341 | cagactaact cgggaataga atatgagtac aaaatgggca gtatgcagat gctgaactaa |
| 2401 | ggctgtggta gatacctttt caaagtttgc attcccagat ttttaaacca aggatcgttt |
| 2461 | cctaactcta ataggcagca aaacgtaagc aggtctctaa caaaaacata acagtagatt |
| 2521 | ccttatctaa attaggatct acacaattag ttaattgaca agaattacaa tgaatataaa |
| 2581 | aagacttgcc aagattggcg atcttaactc ttaaaattat ctaacaataa gaatataggt |
| 2641 | aaggtacaca aactttcata gctataagga gctgacctga aaggccaaaa acagtgtctc |
| 2701 | tgacaaaagc atcttgtaca ttctctgtac cagtcttttt gcctcatgag tcagcttttt |
| 2761 | tagttgtttt tattttaagt tggcaccagg ttggtactcc ttgctgcagc ccatggcgga |
| 2821 | gatacgaagt ttctttatct gtttgtaagt ggctgctctc tgatttctct tcttttgtat |
| 2881 | actcaacata gatttctggt caccactgtc agggcactca caggtcacag gtcagcctgt |
| 2941 | cacattggaa gatagcatga tcttgtagca ttctgtggaa aaaacagaaa cattctctct |
| 3001 | tttccccata ttaagtatct gaacaggatc atggcaagtg ccaataagtg gatccttttt |
| 3061 | atctgtccta gacatcatta tatctagttt gttttttttt tgtaaataaa aatgtgattt |
| 3121 | tatgtgcaca gggatataat tcctaccttc tttgttttta aagaaggtat agtttttaaa |
| 3181 | gttttacaat accttgtctt tgagaattat aaaatatctc agtaacatgt gtaacattaa |
| 3241 | attgttaaca aaacatctct tggaggtttt gaaaataaaa attttgaagc |

SEQ ID NO: 213 Mouse CD84 Isoform 1 Amino Acid Sequence (NP_038517.1)

| | |
|---|---|
| 1 | maqrhlwiwf lclqtwseaa gkdadpvvmn gilgesvtfl lniqepkkid niawtsqssv |
| 61 | afikpgvnka evtitqgtyk grieiidqky dlvirdlrme dagtykadin eeneetitki |
| 121 | yylhiyrrlk tpkitqslis slnntcnitl tcsvekeekd vtyswspfge ksnvlqivhs |
| 181 | pmdqkltytc taqnpvsnss dsvtvqqpct dtpsfhprha vlpgglavlf llilipmlaf |
| 241 | lfrlykrrrd rivleaddvs kktvyavvsr naqptesriy deipqskmls ckkdpvttiy |
| 301 | ssvqlsekmk etnmkdrslp kalgneivv |

SEQ ID NO: 214 Mouse CD84 Transcript Variant 2 cDNA Sequence
(NM_001252472.1; CDS: 180-602)

| | |
|---|---|
| 1 | agtgcttgga gttcctctgt gactgaccac ttcttccttt tctgtctaat ggtgaacacc |
| 61 | tttctggacc agctctggac cagaatctga tttatgctct gctccggaaa caccacactg |
| 121 | aagtgaaagc agctaccaca ccagttattt ttcctcagaa gactggagtc tgactggaca |
| 181 | tggcccagcg ccatctgtgg atctggttcc tttgcctaca aacctggtct gaagcagcag |
| 241 | gaaaagatgc agacccggtg gtaatgaatg ggattcttgg ggagtcagtt actttcctct |
| 301 | taaatattca agaaccaaag aaaattgaca acattgcctg gacttctcaa tcatctgttg |
| 361 | cttttataaa accaggagtc aataaagctg aagttaccat aacccagggc acttataaag |
| 421 | gacgaataga aatcatagat cagaagtatg acctggtcat tagagacctg aggatggaag |
| 481 | atgcaggaac ttacaaagca gacatcaatg aagagaatga ggaaaccatc accaagatct |
| 541 | actaccttca tatctaccgt aagttatggc agcacggggc cttggattta cttttgattt |
| 601 | gaagatttat gatctaaacc acacccatat ttctgataac agagtttcct aactcttctt |
| 661 | atccttataa ttacataagc acatcagtac ttataaaggt ctaactttac ttctggcctt |
| 721 | gacagacttt agctgtaatc tgttttgcag cagaatttgt cctctgttct ttgttttcct |
| 781 | ttcctataaa atgtcaataa tcatattaat caatttgtaa gcattattat gacattctag |
| 841 | taagaaacta tatgcagagt ggtctttata aggcttctgc ttttaagata attacagaat |
| 901 | gcataggaaa atgcaaagaa ctatgaaagg atgtgttctg tgcccttctc ttgccctctc |
| 961 | ctctgttata ctagatccaa actcttagtc tcatcccccg tcttatacag accttctggg |
| 1021 | gtagcctttc ttcattgtct ctcctgattc caaagagata aaataagcag atcctggcac |
| 1081 | acacctttga ttccagcact caggaggcaa agacagaagg atctctatga gttcaaggct |
| 1141 | agcttggtct tcagagaaag ttccagaaca gccaagacta acaaaaagaa accctttctc |
| 1201 | aaaccaacct cccatcccca tcctcacccc ccaaaaacta ctagaatgaa agaaaaaaaa |
| 1261 | ataccaagac acaaaatgaa cagtgtctga ggaactctta tttctgtaag tattaatact |
| 1321 | ttcagaaatg cagaacagtg ttcaaggtca aaaacagaaa attggaactt tcttggaatg |
| 1381 | tgccagcact tacttaggaa tggaatcact tatattccta tagaatttaa tcacatatat |
| 1441 | agcaccggac agcattaatc acatatatag caccggacat cataagcaag gtagtagtga |
| 1501 | agcaccatcc caacattttc ttgtctcagt tctgcagggc agagaactgt tagccctgac |
| 1561 | tccagctttt tctacctaac agttttgtgc aggtgacatc tcgtccctgc tgagaaaatg |
| 1621 | aaatgagatt tagttttcac cactatggct gtaccaatac ctaccccaac gggtggcaca |
| 1681 | cacacacaca cacacacaca cacacacaca cacacacaca cacacacgtc tctccgaaaa |
| 1741 | taggtaaata atgatacttt tcttgtgttt cggctatttt ggaatattca gagcctactg |
| 1801 | ttgtagaata gctgggataa aatggagaca tcctgcccag gctgttattg actgtgcttt |
| 1861 | tttgttggca tctaggcatc tgggactggg atgattataa atctaggtga tgatgtttgg |
| 1921 | atttgtcttt gttgggtggg tggcttgttc cttgttttct gtttcctctc tggattttca |
| 1981 | gagagtgtgg tagctctatg ttttagtagg acattttctt tggatctgac atttatagcc |
| 2041 | actggaggtt ctcaataaaa gatgtttctg ggtattggga gctaacactt aggacctgtg |
| 2101 | ataggttagg agtatgaaag tgttcatagg agagagaaga agagtgttta gccaggatct |
| 2161 | gtttatttca tccccttgga atcagggaga cagtaaagtg aggtcaaccc acagggtctt |
| 2221 | ctctagatac tggggatgag actgggaagt tggatctaga ggaacagaag gaaacaggaa |
| 2281 | gatatacagg ctcctatctg cttctgggca ggaatgatct gggttagcag ggagtgcctg |
| 2341 | ctgaagatga gggctgggat aaaccaacaa gtggggagaa ggtgggtaga aagggaagat |
| 2401 | ctgtggaacc acaatagatg tgggattggg gatgtgcagg agtggggaca ttagaaggaa |
| 2461 | ggctgcagca ggtgttctgt tgcagagcta gggatgaagc tggagcttta gatttgtagg |
| 2521 | agaggaagcc ttctgttagc ttacatgttt cccaggcctg cttgggtggt gtgttcacaa |
| 2581 | ggaacactgg ttttggggac ttgtttactg gaatgaattg ggggaaggaa ggttggagta |
| 2641 | gaagatagag gtcctcaaag agaaaattaa taagtccaca aaatccaaac aaacagtgga |
| 2701 | aagaagtgaa taaactgtat aagacttgaa aatgaaaata aaatcaataa agaaaactaa |
| 2761 | aaccgagaga attctagaaa tgaaaatttt aagaatttga acagaaatta cagaggtaaa |
| 2821 | cttcaccaac aaaatatgag agatggaaga gagaatatta ggcattgaag atacaataga |
| 2881 | gaaaatgaat atatcagtca aagaaaatgt taaaccaaaa aaaaaaagtc ctaacaaaaa |
| 2941 | atacaaaaaa tttaggacac taagaaaaga caaaacctaa gactaataga aatataggaa |
| 3001 | ggaaaagaat tctacctcaa aggcccagaa aatatttcaa caaaatcata gaagaaaaat |
| 3061 | tttctcacct aaagaagata gatgcctata aggtatatga agcatatata acaacaaata |
| 3121 | gatttaacaa gaaaataaac tctccttggc acataacagt caaatcacaa agcatacaga |
| 3181 | acaaagaaag aatattaaaa gctacaaggg gaaaaggcca agtagtatat aaatgcagac |
| 3241 | ctagaatgat acctgatttc tcagtgaaga ctctaaaggc caatagagtc tggacaaatg |
| 3301 | tgctataaac tctaagagac cccagaggtc atccctgatt actataccca ccaaaatatc |
| 3361 | caacatccta aatggaaaaa ataggatatt ccataataaa gccaaattta aacaatatct |
| 3421 | gtctacaaat ccatctatag aagatgacag ggcggaaaat tccaaccaaa agagtttaac |
| 3481 | tataccaaat aaaaacaaaa ggaataaaca atctcaaagc |

SEQ ID NO: 215 Mouse CD84 Isoform 2 Amino Acid Sequence (NP_001239401.1)

| | |
|---|---|
| 1 | maqrhlwiwf lclqtwseaa gkdadpvvmn gilqesvtfl lniqepkkid niawtsqssv |
| 61 | afikpgvnka evtitqgtyk grieiidqky dlvirdlrme dagtykadin eeneetitki |
| 121 | yylhiyrklw qhgaldllli |

SEQ ID NO: 216 Mouse CD84 Transcript Variant 3 cDNA Sequence
(NM_001289470.1; CDS: 180-1166)

| | |
|---|---|
| 1 | agtgcttgga gttcctctgt gactgaccac ttcttccttt tctgtctaat ggtgaacacc |
| 61 | tttctggacc agctctggac cagaatctga tttatgctct gctccggaaa caccacactg |
| 121 | aagtgaaagc agctaccaca ccagttattt ttcctcagaa gactggagtc tgactggaca |
| 181 | tggcccagcg ccatctgtgg atctggttcc tttgcctaca aacctggtct ccagcagcag |
| 241 | gaaaagatgc agacccggtg gtaatgaatg ggattattgg ggagtcagtt actttcctct |
| 301 | taaatattca agaaccaaag aaaattgaca acattgcctg gacttctcaa tcatctgttg |
| 361 | cttttataaa accaggagtc aataaagctg aagttaccat aacccagggc acttataaag |
| 421 | gacgaataga aatcatagat cagaagtatg acctggtcat tagagacctg aggatggaag |
| 481 | atgcaggaac ttacaaagca gacatcaatg aagagaatga ggaaaccatc accaagatct |
| 541 | actaccttca tatctaccgt cgacttaaaa caccaaaaat tacacagagt ttgatatcat |
| 601 | ctttgaacaa tacctgtaat atcacactga catgctctgt ggaaaaggaa gaaaaggatg |
| 661 | tcacatatag ctggagtccc tttggagaga aaagcaatgt ccttcaaatc gtccactccc |
| 721 | ccatggacca aaaactgacc tacacatgta cagcccagaa ccctgtcagc aacagttctg |
| 781 | actctgtcac tgtccagcag ccatgtacag acactccaag cttccatcct cgccatgctg |
| 841 | tgttgccagg aggattggcc gtgctctttc tgcttattct cattccgatg ttggcatttc |
| 901 | tgttccgttt gtataagaga aggcgagaca ggattgtcct ggaagatgat gtctcaaaga |
| 961 | aaacagtata tgctgtagtt tcaagaaatg ctcaacccac agagtccaga atctatgatg |
| 1021 | aaatccctca gtccaagatg ctgtcctgta agaaagatcc ggtgaccacc atttattcct |
| 1081 | cagtgcagct ttctgagaag atgaaggaaa ccaacatgaa ggacagaagt ctgcctaagg |
| 1141 | ctttgggtaa tgaaattgtt gtctaggtga ttctctaaga ccacgaagga cacaaggaca |
| 1201 | agtcatctat gaggattgaa tcaacggttt cagtcttttg gatataacct gggccagcca |
| 1261 | agggatttag gaatgaagca agctccgtgg gtagaggtct gatccccagt gtgtaatgtt |
| 1321 | aggggccatg tacaggattg actctcaggc ccacagatct ttacccagag aaaccctgac |
| 1381 | ctgctcccat gctgtttttc ctggggaaag gaccctaggg cactcaacct ttatgcaatc |
| 1441 | agacatgcct ctcagagact gtctaacagc ttccagacta atctctgtgc agtacttagt |
| 1501 | cttacaactc tcacgggcaa cggcttcaag ttccaatttt acgatgtgtc tagcctggga |
| 1561 | tgactgttta gtttctaatg tggcgagaat gtatgttacc atgtaggaag cacagactat |
| 1621 | ggcaatctat aaatgatttg tggcatgaga ctgatgtccg aatttagggg aaggggaatg |
| 1681 | gtcttactta ggcattttat ggaaattgag tctctctccc cgagaagagg gtgatgaagc |
| 1741 | agcatccacg tctgcctctt ctccagtaac ctgcttgtta tcgacaatgt ccagccgatg |
| 1801 | gtaatgaact gaaacaaatg cgcttgaaag agatgaatca atttgagatt taacaaatcg |
| 1861 | ggtcaatttc tgaaatgccc aaggaccgaa ggagatcaat aataggagtc ccaatagggg |
| 1921 | ccctaaaagg gagggcaaca aggttgaaag tcagggggag gttgaaaacc agttttggta |
| 1981 | gtctaccttc cccctaggcc attgtaaata cttgtgtatg ggtgtaactc agctatctat |
| 2041 | agttctaagt atccactctg gttcctcttt aggtcttaaa cttccttctt cctagttgat |
| 2101 | ggtaaattcc tgtgtaaggc agtggcctag cttttattca aagtgatagt gtaatgccag |
| 2161 | aggctattct gaatgtcact gaataggcaa cactctccct gaattctaag tccatggtct |
| 2221 | gttcaagggc tttttaggac attggaacac cagtgaaggc ttagctatgt cagaattcaa |
| 2281 | tcttaaaatg cacttataat aagataatat taaaagagag cacatggatc tatacaccag |
| 2341 | actaactcgg gaatagaata tgagtacaaa atgggcagta tgcagctgct gaactaaggc |
| 2401 | tgtggtagat accttttcaa agtttgcctt cccagatttt taaaccaagg atcgtttcct |
| 2461 | aactctaata ggcagcaaaa cgtaagcagg tctctaacaa aaacataaca gtagattcct |
| 2521 | tatctaaatt aggatctaca caattagtta attgacaaga attacaatga atataaaaag |
| 2581 | acttgccaag attggcgatc ttaactctta aaattatcta acaataagca tataggtaag |
| 2641 | gtacacaaac tttcatagct ataaggagct gacctgaaag gccaaaaaca gtgtctctga |
| 2701 | caaaagcatc ttgtacattc tctgtaccag tctttttgcc tcatgagtca gcttttttag |
| 2761 | ttgtttttat tttaagttgg caccaggttg gtactccttg ctgcagccca tggcggagat |
| 2821 | acgaagtttc tttatctgtt tgtaagtggc tgctctctga tttctcttct tttgtatact |
| 2881 | caacatagct ttctggtcac cactgtcagg gcactcacag gtcacaggtc agcctgtcac |
| 2941 | attggaagct agcatgctct tgtagcattc tgtggaaaaa acagaaacat tctccctttt |
| 3001 | ccccatatta agtatctgaa caggatcatg gcaagtgcca ataagtggat cctttttatc |
| 3061 | tgtcctagac atcattatat ctagtttgtt ttttttttgt aaataaaaat gtgattttat |
| 3121 | gtgcacaggg atataattcc taccttcttt gtttttaaag aaggtatagt ttttaaagtt |
| 3181 | ttacaatacc ttgtctttga gaattataaa atatctcagt aacatgtgta acattaaatt |
| 3241 | gttaacaaaa catctcttgg aggttttgaa tataaaaatt ttgaagc |

SEQ ID NO: 217 Mouse CD84 Isoform 3 Amino Acid Sequence (NP_001276399.1)

| | |
|---|---|
| 1 | maqrhlwiwf lclqtwseaa gkdadpvvmn gilgesvtfl lniqepkkid niawtsqssv |
| 61 | afikpgvnka evtitqgtyk grieiidqky dlvirdlrme dagtykadin eeneetitki |
| 121 | yylhiyrrlk tpkitqslis slnntcnitl tcsvekeekd vtyswspfge ksnvqqivhs |
| 181 | pmdqkltytc taqnpvsnss dsvtvqqpct dtpsfhprha vlpgglavif llilipmlaf |
| 241 | lfrlykrrrd rivleddvsk ktvyavvsrn aqptesriyd eipqskmlsc kkdpvttiys |
| 301 | svqlsekmke tnmkdrslpk algneivv |

*The nucleic acid and polypeptide sequences of the biomarkers encompassed by the present invention listed in Table 2 have been submitted at GenBank under the unique identifier provided herein and each such uniquely identified sequence submitted at GenBank is hereby incorporated in its entirety by reference.
*included in Table 2 are RNA nucleic acid molecules e.g., thymidines replaced with uridines), nucleic acid molecules encoding orthologs of the encoded proteins, as well as DNA or RNA nucleic acid sequences comprising a nucleic acid sequence having at least 80%, 81%, 87%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any publicly available sequence listed in Table 2, or a portion thereof Such nucleic acid molecules can have a function of the full-length nucleic acid as described further herein.
*Included in Table 2 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any publicly available sequence listed in Table 2, or a portion thereof. Such polypeptides can have a function of the full-length polypeptide as described further herein.
*Included in Table 2 are additional known nucleic acid and amino acid sequences for the listed biomarkers.

### IV. Agents Useful for Modulating Targets and Biomarkers

It is demonstrated herein that the inflammatory phenotype of monocytes and/or macrophages can be controlled by modulating the copy number, amount, and/or activity of certain biomarkers (e.g., at least one target listed in Table 1 and/or Table 2), either alone or in combination and that the modulation of the inflammatory phenotype can modulate immune responses. Thus, the present invention provides compositions that modulate the copy number, amount, and/or activity of at least one biomarker (e.g., at least one target listed in Table 1 and/or Table 2) can upregulate or downregulate the inflammatory phenotype and, thereby, upregulate or downregulate, respectively, an immune response. Agents are also described herein that can detect the copy number, amount, and/or activity of the at least one biomarker (e.g., at least one target listed in Table 1 and/or Table 2), such that the agents are useful for diagnosing, prognosing, and screening effects mediated by the at least one biomarker (e.g., at least one target listed in Table 1 and/or Table 2).

An agent that downregulates the copy number, amount, and/or activity of at least one target listed in Table 1, such as by agents that downregulate the at least one target like antibodies, siRNAs, and the like described herein, can increase the inflammatory phenotype of monocytes and/or macrophages.

An agent that downregulates the copy number, amount, and/or activity of at least one target listed in Table 2, such as agents that downregulate the at least one target like antibodies, siRNAs, and the like described herein, can decrease the inflammatory phenotype of monocytes and/or macrophages.

Any agent that modulates the at least one biomarker described herein (e.g., at least one target listed in Table 1 and/or Table 2) is encompassed by the present invention. The agent can modulate genetic sequence, copy number, gene expression, translation, post-translational modification, subcellular localization, degradation, conformation, stability, secretion, enzymatic activity, transcription factors, receptor activation, signal transduction, and other biochemical functions mediated by the at least one biomarker.

The agent can bind any cell moiety, such as a receptor, a cell membrane, an antigenic determinant, or other binding site present on a target molecule or a target cell. In some embodiments, the agent can diffuse or be transported into the cell, where it can act intracellularly. In some embodiments, the agent is cell-based.

As described further below, representative agents include, without limitation, nucleic acids (DNA and RNA), oligonucleotides, polypeptides, peptides, antibodies, fusion proteins, antibiotics, small molecules, lipids/fats, sugars, vectors, conjugates, vaccines, gene therapy agents, cell therapy agents, and the like, such as a small molecule, mRNA encoding a polypeptide, CRISPR guide RNA (gRNA), RNA interfering agent, small interfering RNA (siRNA), CRISPR RNA (crRNA and tracrRNA), a small hairpin RNA (shRNA), a microRNA (miRNA), a piwi-interacting RNA (piRNA), antisense oligonucleotide, peptide or peptidomimetic inhibitor, aptamer, natural ligands and derivative thereof that bind and either activate or inhibit protein biomarkers, antibody, intrabody, or cells, either alone or in combination with other agents.

In some embodiments, agents that modulate the interaction between at least one biomarker (e.g., at least one target listed in Table 1 and/or Table 2) and a natural binding partner are useful according to the present invention. For example, in one embodiment, agents which directly block an interaction(s) between a biomarker and one or more of its natural binding partners (e.g., blocking antibodies) can modulate biomarker activity and thereby modulate inflammatory phenotype. Alternatively, agents that indirectly block the interaction(s) are useful. For example, a soluble protein, by binding to a biomarker natural binding partner or, alternatively, by mimicking the natural binding partner of the biomarker indirectly reduces the effective concentration of biomarker and/or biomarker natural binding partner available to bind to the respective protein on cells. Exemplary agents include antibodies against the biomarker or natural binding partner(s) of the biomarker that block the interaction between the biomarker and the natural binding partner(s); a non-activating form of the biomarker and/or natural binding partner(s) of the biomarker (e.g., a dominant negative polypeptide), small molecules or peptides that block the interaction between the biomarker and natural binding partner(s) thereof; fusion proteins (e.g., the extracellular portion of the biomarker and/or natural binding partner(s) thereof fused to the Fc portion of an antibody or immunoglobulin) that inhibit the interaction between the biomarker and natural binding partner(s) thereof; nucleic acid molecules and/or genetic modifications that block transcription or translation of the biomarker and/or the natural binding partner(s) thereof; a non-activating form of a biomarker and/or natural binding partner(s) thereof.

In other exemplary embodiments, agents that promote the binding of a biomarker (e.g., one or more targets listed in Table 1 and/or Table 2) to one or more natural binding partners are encompassed by the present invention. Agents that modulate such an interaction can do so either directly or indirectly. Thus, in one embodiment, agents which directly enhance the interaction between a biomarker and natural binding partner(s) of the biomarker are useful modulatory agents. Alternatively, agents that block binding of a biomarker and/or natural binding partner(s) of the biomarker to other binding partners increase the effective concentration of the two components available to bind to each other. Exemplary agents include antibodies against a biomarker and/or natural binding partner(s) thereof, small molecules, and peptides that activate or promote the interaction between the biomarker and natural binding partner(s) thereof.

Agents encompassed by the present invention can comprise any number, type, and modality. For example, agents can comprise 1, 2, 3, 4, 5, or more, or any range in between, inclusive, number of agents that modulates a biomarker or more than on biomarker (e.g., 2 agents that modulate the same target listed in Table 1 or Table 2, one agent that modulates a target listed in Table 1 and a second agent that modulates a target listed in Table 2, a combination of an siRNA and an antibody agent that modulates a target listed in Table 2, a combination of two siRNAs that modulates a single target listed in Table 1 along with a single siRNA that modulates a single target listed in Table 2 and an antibody agent that modulates a different target listed in Table 2, etc.).

In some embodiments, modulatory agents encompassed by the present invention further comprise one or more additional agents that target phagocytes, e.g., monocytes and/or macrophages. Such monocyte/macrophage targeting agents include, but are not limited to, rovelizumab which targets CD11b, small molecules, including NRP1685A (which targets Neurophilin-1), nesvcumab targeting ANG2, pascolizumab specific to IL-4, dupilumab specific to IL4Rα, tocilizumab and sarilumab specific to IL-6R, adalimumab, certolizumab, tanercept, golimumab, and infliximab specific to TNF-α, and CP-870 and CP-893 targeting CD40.

In addition to agents described below and herein, exemplary agents for modulating biomarkers of interest encompassed by the present invention are described in the art (see, e.g., (i) a co-pending application filed by Novobrantseva et al. (Verseau Therapeutics, Inc.) on Jun. 4, 2019 as U.S. Ser. No. 62/857,169 having the title “Anti-PSGL-1 Compositions and Methods for Modulating Monocyte and Macrophage Inflammatory Phenotypes and Uses Thereof;” (ii) a co-pending application filed by Novobrantseva et al. (Verseau Therapeutics, Inc.) on Jun. 27, 2019 as U.S. Ser. No. 62/867,569 having the title “Anti-PSGL-1 Compositions and Methods for Modulating Monocyte and Macrophage Inflammatory Phenotypes and Uses Thereof;” (iii) a co-pending application filed by Novobrantseva et al. (Verseau Therapeutics, Inc.) on Jun. 4, 2019 as U.S. Ser. No. 62/857,194 having the title “Anti-SIGLEC-9 Compositions and Methods for Modulating Monocyte and Macrophage Inflammatory Phenotypes and Uses Thereof;” (iv) a co-pending application filed by Novobrantseva et al. (Verseau Therapeutics, Inc.) on Jun. 27, 2019 as U.S. Ser. No. 62/867,577 having the title “Anti-SIGLEC-9 Compositions and Methods for Modulating Monocyte and Macrophage Inflammatory Phenotypes and Uses Thereof;” (v) a co-pending application filed by Novobrantseva et al. (Verseau Therapeutics, Inc.) on Jun. 27, 2019 as U.S. Ser. No. 62/867,593 having the title “Anti-LRRC25 Compositions and Methods for Modulating Monocyte and Macrophage Inflammatory Phenotypes and Uses Thereof;” and (vi) a co-pending application filed by Novobrantseva et al. (Verseau Therapeutics, Inc.) on Jun. 27, 2019 as U.S. Ser. No. 62/867,602 having the title “Anti-CD53 Compositions and Methods for Modulating Monocyte and Macrophage Inflammatory Phenotypes and Uses Thereof;” the entire contents of each of said applications being incorporated herein in their entirety by this reference).

1. Nucleic Acid Agents

One aspect encompassed by the present invention involves the use of nucleic acid molecules. Nucleic acid molecules can be deoxyribonucleic acid (DNA) molecules (e.g., cDNA, genomic DNA, and the like), ribonucleic acid (RNA) molecules (e.g., mRNA, long non-coding RNA, small RNA species, and the like), DNA/RNA hybrids, and analogs of the DNA or RNA generated using nucleotide analogs. RNA agents can include RNAi (RNA interfering) agents (e.g., small interfering RNA (siRNA)), single-strand RNA (ssRNA) molecules (e.g., antisense oligonucleotides) or double-stranded RNA (dsRNA) molecules. A dsRNA molecule comprises a first strand and a second strand, wherein the second strand is substantially complementary to the first strand, and the first strand and the second strand form at least one double-stranded duplex region. The dsRNA molecule can be blunt-ended or have at least one terminal overhang. When used as agents that bind target nucleic acid sequences, nucleic acid agents encompassed by the present invention can n hybridize to any region of a target sequence, such as genomic sequence and/or mRNA sequence, including, but not limited to, the enhancer region, the promoter region, the transcriptional start and/or stop region, splice sites, the coding region, the 3′-untranslated region (3′-UTR), the 5′-untranslated region (5′-UTR), the 5′ cap, the 3′ poly adenylyl tail, or any combination thereof.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A nucleic acid molecule encompassed by the present invention can be isolated using standard molecular biology techniques and the sequence information in the database records described herein. Using all or a portion of such nucleic acid sequences, nucleic acid molecules encompassed by the present invention can be isolated using standard hybridization and cloning techniques (e.g., as described in Sambrook et al., ed., A Molecular Cloning: A Laboratory Manual, 4th ed., Cold Spring Harbor Laboratory Press, Cold Spring Harbor, N.Y., 2012).

A nucleic acid molecule encompassed by the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, nucleic acid molecules corresponding to all or a portion of a nucleic acid molecule encompassed by the present invention can be prepared by standard synthetic techniques, e.g., using an automated nucleic acid synthesizer. Alternatively, the nucleic acid molecules can be produced biologically using an expression vector into which a nucleic acid has been sub-cloned. For example, antisense nucleic acid molecules can be cloned in an antisense orientation (i.e., RNA transcribed from the inserted nucleic acid will be of an antisense orientation to a target nucleic acid of interest as described further below).

Moreover, a nucleic acid molecule encompassed by the present invention can comprise only a portion of a nucleic acid sequence, wherein the full length nucleic acid sequence comprises a marker encompassed by the present invention or which encodes a polypeptide corresponding to a marker encompassed by the present invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers encompassed by the present invention. The probe comprises a label group attached thereto, e.g., a radioisotope, a fluorescent compound, an enzyme, or an enzyme co-factor.

Biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that can affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's can occur in protein-coding nucleic acid sequences, in which case they can give rise to a defective or otherwise variant protein, or genetic disease. Such a SNP can alter the coding sequence of the gene and therefore specify another amino acid (a “missense” SNP) or a SNP can introduce a stop codon (a “nonsense” SNP). When a SNP does not alter the amino acid sequence of a protein, the SNP is called “silent.” SNP's can also occur in noncoding regions of the nucleotide sequence. This can result in defective protein expression, e.g., as a result of alternative spicing, or it can have no effect on the function of the protein.

As used herein, the terms “gene” and “recombinant gene” refer to nucleic acid molecules comprising an open reading frame encoding a polypeptide corresponding to a marker encompassed by the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope encompassed by the present invention.

In another embodiment, a biomarker nucleic acid molecule can be at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker encompassed by the present invention or to a nucleic acid molecule encoding a protein corresponding to a marker encompassed by the present invention. The term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule encompassed by the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species can be non-essential for activity and thus would be likely targets for alteration. Alternatively, amino acid residues that are conserved among the homologs of various species (e.g., murine and human) can be essential for activity and thus would not be likely targets for alteration.

Accordingly, another aspect encompassed by the present invention encompasses nucleic acid molecules encoding a polypeptide encompassed by the present invention that contain changes in amino acid residues that are not essential for activity. Such polypeptides differ in amino acid sequence from the naturally-occurring proteins which correspond to the markers encompassed by the present invention, yet retain biological activity. In one embodiment, a biomarker protein has an amino acid sequence that is at least about 40%/identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids encompassed by the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

As described further below, some forms of nucleic acids useful according to the present invention can act as inhibitors, which refers to an agent that inhibits the function of a biological target. In some embodiments, the inhibitor is a gene silencing agent that prevents the expression of a gene or gene product. “Gene silencing” is often referred to as “gene knockdown.” Gene silencing can occur on the transcriptional level, i.e., prevent the transcription of DNA to RNA, or on the translational level, i.e., post-transcriptional silencing i.e., prevent the translation of mRNA to protein. Types of transcriptional gene silencing include genomic imprinting, paramutation, transposon silencing, histone modification, transgene silencing, position effect, and RNA-directed DNA methylation, for example. Examples of post-transcriptional gene silencing include RNA interference (RNAi), RNA silencing, and nonsense mediated decay. A gene silencing agent can be designed to silence (e.g., inhibit the expression of) a specific gene or to silence multiple genes simultaneously. A gene silencing agent can reduce the expression of a gene and/or gene product by at least about 20%, at least about 30%, at least about 40%, at least about 50%, at least about 60%, at least about 70%, at least about 80%, at least about 90%, at least about 95%, at least about 98%, at least about 99%, or at least about 100%. In some embodiments, a gene silencing agent reduces expression of a gene and/or gene product by at least about 70%.

In some embodiments, nucleic acids in genomes are useful and can be used as targets and/or agents. For example, target DNA in the genome can be manipulated using well-known methods in the art. Target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, can be altered by site-directed mutagenesis.

a. Messenger RNA (mRNA) and cDNA

In some embodiments, mRNAs and/or cDNA that encode target proteins and variants thereof can be used as agents to modulate target protein amount and/or activity of interest. mRNA and cDNA can be modified to increase the stability and/or immunogenicity, for example, codon optimization.

b. Small Interfering RNA (siRNA)

In some embodiments, a nucleic acid agent can be an RNAi (RNA interference) agent. An RNAi agent can be a single stranded RNA molecule, or a double-stranded RNA molecule such as small (or short) interfering RNA (siRNA) molecule. A siRNA molecule is a double-stranded oligonucleotide or RNA molecules having a sense strand and an antisense strand wherein the antisense strand is substantially complementary to a sequence in a target mRNA molecule. A siRNA molecule upon cellular delivery will induce RNA interference (RNAi). RNAi is a post-transcriptional mechanism of gene silencing through chromatin remodeling, inhibition of protein translation, or direct mRNA degradation. During the RNAi process, small RNA molecules such as siRNAs are recruited to the RNA-induced silencing complex (RISC). This complex is able, via the siRNA molecules, to bind to substantially complementary sequences (i.e., the mRNA of a transcribed gene) and degrade them by endonuclease activity. This leads ultimately to inhibition of expression of the corresponding gene that encodes the mRNA complementary to the siRNA molecules (e.g., McManus and Sharp (2002) Nat. Rev. Genet. 3: 737-747).

The term “double stranded RNA,” a “duplex RNA,” or a “RNA duplex” refers to an RNA of two strands and with at least one double-stranded region, and includes RNA molecules that have at least one gap, nick, bulge, loop, and/or bubble either within a double-stranded region or between two neighboring double-stranded regions. If one strand has a gap or a single-stranded region of unmatched nucleotides between two double-stranded regions, that strand is considered as having multiple fragments. A double-stranded RNA as used here can have terminal overhangs on either end or both ends. In some embodiments, the two strands of the duplex RNA can be linked through certain chemical linker.

The term “antisense strand” refers to an RNA strand that has substantial sequence complementarity against a target messenger RNA. An antisense strand can be part of a siRNA molecule, part of a miRNA/miRNA duplex, or a single-strand mature miRNA.

The sense and antisense strand of a siRNA molecule each can comprise about 10 to 50 nucleotides or nucleotide analogs. Preferably, the sense and antisense strand of the siRNA molecule each has a length from about 15-45 nucleotides. Further preferably, the antisense and the sense strand of the siRNA molecule each has a length from 18 to 30 nucleotides, or from 21 to 23 nucleotides, for example, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, or about 30 nucleotides.

The sense and antisense strands of a siRNA molecule form a duplex region. The antisense strand comprises (or alternatively, consists essentially of, or consists of) a nucleotide sequence that is substantially complementary to a target mRNA to mediate RNAi.

The term “substantially complementary” refers to complementarity in a based-paired and double stranded region of the siRNA molecule. The complementarity does not need to be perfect; there can be any number of base pair mismatches that do not impact hybridization under even the least stringent hybridization conditions. For example, the antisense region of the siRNA molecule encompassed by the present invention can comprise at least about 70% or greater complementary, at least about 75% or greater complementary, at least about 80% or greater complementary, or at least about 85% or greater complementary, or at least about 90% or greater complementary, or at least about 91% or greater complementary, or at least about 92% or greater complementary, or at least about 93% or greater complementary, or at least about 94% or greater complementary, or at least about 95% or greater complementary, or at least about 96% or greater complementary, or at least about 97% or greater complementary, or at least about 98% or greater complementary, or at least about 99% or greater complementary, to the nucleic acid sequence of the target mRNA molecule.b

siRNA molecules can further include at least one overhang region, wherein each overhang region has six or fewer nucleotides. For example, when the antisense and sense strands of a siRNA molecule are aligned, there are at least one, two, three, four, five or six nucleotides at the end of the strands which do not align (i.e., no complementary bases in the opposing strand). In some examples, an overhang can occur at one or both ends of the duplex when the sense and antisense strands are annealed.

In some examples, the antisense region and the sense region of the siRNA molecule can vary in lengths, sequences and the nature of chemical modifications thereto.

c. MicroRNA (miRNA) and Piwi-Interacting RNA (piRNA)

In some embodiments, nucleic acid molecules can be miRNAs, miRNA mimetics, or miRNA inhibitors. miRNAs are a class of naturally occurring, small noncoding RNA molecules 21-25 nucleotides in length that regulate gene expression post-transcriptionally and part of the cell's RNAi mechanism. miRNAs are partially complementary to messenger RNA (mRNA) molecules, and their main function is down-regulation of gene expression via translational repression, mRNA cleavage and deadenylation.

MicroRNA inhibitors are antagomirs, which can be used in the silencing of endogenous miRNAs. miRNA mimetics or mimics are miRNA agonists, and can be used to replace endogenous miRNAs as functional equivalents and thereby up-regulating pathways affected by such endogenous miRNAs.

“Piwi-interacting RNA (piRNA)” is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

d. Antisense Nucleic Acids and Oligonucleotides

In some embodiments, nucleic acid molecules can comprise antisense nucleic acid molecules, such as those having a sequence complementary to a target mRNA and/or complementary to the coding strand of a double-stranded cDNA. An antisense nucleic acid molecule encompassed by the present invention can hydrogen bond to (i.e. anneal with) can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of interest. The non-coding regions (“5′ and 3′ untranslated regions”) are the 5′ and 3′ sequences which flank the coding region and are not translated into amino acids.

In some embodiments, nucleic acid molecules can comprise oligonucleotides, including antisense and sense oligonucleotides. Oligonucleotides are short single-strand nucleic acid molecules that upon cellular uptake can selectively inhibit the expression and function of a target protein. Antisense oligonucleotides are complementary to target mRNA and/or complementary to the coding strand of a double-stranded cDNA and typically have 10-50 nucleotides in length, preferably 15-30 nucleotides in length, more preferably 18-20 nucleotides in length. For example, the antisense oligonucleotide can comprise 18 nucleotides, or 19 nucleotides, or 20 nucleotides, or 21 nucleotides, or 22 nucleotides, or 23 nucleotides, or 24 nucleotides, or 25 nucleotides, or 26 nucleotides, or 27 nucleotides, or 28 nucleotides, or 29 nucleotides, or 30 nucleotides. Antisense oligonucleotides can form a duplex with the target mRNA and inhibit its translation or processing, consequently inhibiting protein biosynthesis. Antisense oligonucleotides are preferably designed to target the initiator codons, the transcriptional start site of the targeted gene or the intron-exon junctions. For therapeutic purpose, oligonucleotides can be used to selectively block the expression of target proteins associated with macrophages that are implicated in the diseases.

Antisense oligonucleotides can inhibit gene expression through various mechanisms: (1) degradation of the complexes between target RNA/DNA oligonucleotide by RNase H. The latter is a ubiquitous nuclear enzyme required for DNA synthesis, which functions as an endonuclease that recognizes and cleaves the RNA in the duplex. Most types of oligonucleotides, but not all, from complexes with mRNA that direct the cleavage by RNase H: (2) inhibition of translation by the ribosomal complexes; (3) competition for mRNA splicing when oligonucleotides are designed against intron-exon junctions.

e. Ribozymes and DNAzymes

In some embodiments, nucleic acid molecules can be ribozymes and DNAzymes. Ribozymes are single stranded RNA molecules retaining catalytic activities which are capable of sequence specific cleaving of RNA molecules (see, e.g., Haselhoff and Gerlach (1988) Nature 334:585-591). They function by binding to the target through antisense sequence specific hybridization and inactivating it by cleaving the phosphodiester backbone at a specific site. Their structures are based on naturally occurring site-specific, self-cleaving RNA molecules. Five classes of ribozymes have been described based on their unique characters, i.e., the Tetrahymena group I intron, RNase P, the hammerhead ribozyme, the hairpin ribozyme and the hepatitis delta virus ribozyme. Hammerhead ribozymes cleave RNA at the nucleotide sequence U-H (H=A, C or U) by hydrolysis if a 3′-5′ phosphodiester bond. Hairpin ribozymes utilize the nucleotide sequence C-U-G as their cleavage site. In some embodiments, ribozymes can be used for knocking out therapy by targeting overexpressed genes in cells of interest. A ribozyme having specificity for a nucleic acid molecule encoding a polypeptide corresponding to a marker encompassed by the present invention can be designed based upon the nucleotide sequence of a cDNA corresponding to the marker. For example, a derivative of a ltrahymena L-19 IVS RNA can be constructed in which the nucleotide sequence of the active site is complementary to the nucleotide sequence to be cleaved (see, e.g., U.S. Pat. Nos. 4,987,071 and 5,116,742). Alternatively, an mRNA encoding a polypeptide of interest can be used to select a catalytic RNA having a specific ribonuclease activity from a pool of RNA molecules (see, e.g., Bartel and Szostak (1993) Science 261:1411-1418).

DNAzymes are analogs of ribozymes with greater biological stability in which the RNA backbone is replaced by DNA motifs that confer improved biological stability.

f. Aptamers

In some embodiments, nucleic acid molecules can be aptamers. DNA or RNA aptamers are double stranded (i.e. DNA aptamers) or single stranded (i.e., RNA aptamers) nucleic acid segments that can directly interact with target proteins and interfere their activities. Generally, “aptamers” are oligonucleotide or peptide molecules that bind to a specific target molecule. “Nucleic acid aptamers” are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In some embodiments, aptamers can be used to modulate the molecular functions of the target proteins of macrophages as implicated in the diseases. In some instances, aptamers are preferred over antibodies in protein inhibition owing to their specificity and affinity to the target protein, non-immunogenicity, and stability of pharmaceutical formulations.

g. Nucleic Acid Decoys

In some embodiments, nucleic acid molecules can be decoy DNAs or decoy RNAs. Nucleic acid decoys are particularly useful for targeting transcription factors. RNA decoys are specifically designed small RNA molecules to provide alternate, competing binding sites for proteins that act as translational activators or mRNA stabilizing elements. RNA decoys can be used to prevent translation or induce instability and, ultimately destruction of the mRNA molecules. In some examples, overexpressed short RNA molecules corresponding to critical cis-acting regulatory elements can be used as decoys for trans-activating proteins, thus preventing binding of these trans-activators to their corresponding cis-acting elements.

In other examples, decoys can be double-stranded nucleic acid molecules (e.g., DNA) with high binding affinity for the targeted proteins particularly, transcription factors which are sequence-specific double-stranded DNA binding proteins which modulate (increase or decrease) the rate of transcription of one or more specific genes in the macrophage.

h. Nucleic Acid Chimeras

In some embodiments, nucleic acid molecules can be nucleic acid chimeras. Nucleic acid chimeras are conjugates of different types of nucleic acid molecules which are designed to modulate the macrophage associated target protein. For example, a conjugate of cell internalizing DNA or RNA aptamers that bind to cell surface receptors as carriers and siRNA molecules (or miRNAs) specific to a target protein can be used an approach for macrophage regulation. The aptamer-siRNA chimeras can improve the delivery and therapeutic effect.

i. Triple Helical Structures

In some embodiments, nucleic acid molecules encompassed by the present invention can form triple helical structures. For example, expression of a protein of interest can be inhibited by targeting nucleotide sequences complementary to the regulatory region of the gene encoding the polypeptide (e.g., the promoter and/or enhancer) to form triple helical structures that prevent transcription of the gene in target cells (see, e.g., Helene (1991) Anticancer Drug Des. 6:569-584; Helene (1992) Ann. N.Y. Acad. Sci. 660:27-36, Maher (1992) Bioassays 14:807-815). Such nucleic acids can bind to DNA duplexes through specific interactions in the major groove of the double helix.

j. Nucleic Acid Modifications and Variants

In some embodiments, nucleic acid molecules encompassed by the present invention can contain one or more chemical modifications. The modifications will not compromise the activity of the nucleic acid molecules. Chemical modifications well-known in the art are capable of increasing stability, availability, and/or cell uptake of the nucleic acid molecules. In one embodiment, modifications can be used to provide improved resistance to degradation (by nucleases) or improved uptake of nucleic acid molecules by cells. In some embodiments, modified nucleic acid molecules encompassed by the present invention can have an enhanced target efficiency as compared to corresponding non-modified nucleic acid molecules.

In some embodiments, nucleic acid molecules encompassed by the present invention can be optimized, such as to increase expression, improve the effectiveness of gene silencing for use to silence a target gene, and the like. In another embodiment, modifications can be used to increase or decrease affinity for the complementary nucleotides in the target mRNA and/or in the complementary siRNA strand. In some embodiments, siRNAs encompassed by the present invention can be modified to increase the ability to avoid or modulate an immune response in a cell, tissue or organism.

In some embodiments, nucleic acid molecules encompassed by the present invention can be further modified to increase the membrane penetrance and/or delivery to a target organ, tissue and cell. In one example, the nucleic acid molecule can be modified to increase its delivery to myeloid cells, monocytes and macrophages. For example, nucleic acid molecules can be modified such that they specifically bind to receptors or antigens expressed on a selected cell surface, e.g., by linking the antisense nucleic acid molecules to peptides or antibodies which bind to cell surface receptors or antigens. The nucleic acid molecules can also be modified as part of vectors that target cells of interest and/or selectively express within cells of interest.

Duplex molecules encompassed by the present invention, such as siRNA molecules, can comprise a modified sense strand, a modified anti-sense strand, or modified sense and antisense strands.

In some embodiments, a nucleic acid molecule encompassed by the present invention can be an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

Nucleic acid molecules encompassed by the present invention can be modified at the 5′ end, 3′ end, 5′ and 3′ end, and/or at internal residues, or any combination thereof. As described herein, a naturally occurring nucleic acid with repeating nucleotide residues has a backbone consisting of sugars and phosphodiesters, and nitrogenous bases (often called nucleobases or simply bases). Accordingly chemically modified nucleotides can include modified nucleobases, modified sugars and/or non-phosphodiester linkages (i.e., backbone modifications). In some embodiments, the modification is a mixture of different kinds of modifications described herein, such as a combination of unlocked nucleomonomer agents (UNAs), modified cap structures, modified inter-nucleoside linkages and or nucleobase modifications.

In some embodiments, nucleic acid molecules encompassed by the present invention can further comprise at least one terminal modification or “cap.”

For example, the cap can be a 5′ and/or a 3′-cap structure. The terms “cap” and “end-cap” include chemical modifications at either terminus of each strand of the nucleic acid molecule (with respect to terminal ribonucleotides), and/or modifications at the linkage between the last two nucleotides at the 5′ end and/or the last two nucleotides at the 3′ end. The cap structure can increase resistance of the nucleic acid molecule to exonucleases without compromising molecular interactions with target mRNAs or cellular machinery. Such modifications can be selected on the basis of their increased potency in vitro or in vivo.

The cap can be present at the 5′-terminus (5′-cap) or at the 3′-terminus (3′-cap) or can be present at both ends. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′) (e.g., Invabasic X, Abasic II, rSpacer/RNA abasic), and dSpacer), phosphorodithioate monophosphate, and methylphosphonate moiety. The phosphorothioate or phosphorodithioate linkage(s), when part of a cap structure, are generally positioned between the two terminal nucleotides at the 5′ end and the two terminal nucleotides at the 3′ end.

In some embodiments, nucleic acid molecules encompassed by the present invention have at least one terminal phosphorothioate monophosphate. The phosphorothioate monophosphate can be at the 5′ and/or 3′ end of each strand of the nucleic acid molecule. In other embodiments, the nucleic acid molecule has terminal phosphorothioate monophosphate at both 5′ and 3′ terminus of the sense and/or antisense strand. The phosphorothioate monophosphate can support a higher potency by inhibiting the action of exonucleases.

In some embodiments, modifications at the 5′ end is preferred in the sense strand, and comprises, for example, a 5′-propylamine group. Modifications to the 3′ OH terminus are in the sense strand, antisense strand, or in the sense and antisense strands. A 3′ end modification comprises, for example, 3′-puromycin, 3′-biotin and the like.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof. Exemplary 5′-modifications include, but are not limited to, 5′-monophosphate ((HO)₂(O)P-O-5′); 5′-diphosphate ((HO)₂(O)P—O—P(HO)(O)—O-5′); 5′-triphosphate ((HO)₂(S)P—O—(HO)(O)P—O—P(HO)(O)-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc.), 5′-alkyletherphosphonates (R(OH)(O)P—O-5′, R=alkylether, e.g., methoxymethyl (CH₂OMe), ethoxymethyl, etc.).

In some embodiments, the cap at the terminus of the nucleic acid molecule can be a conjugate, for example, a 5′ conjugate. The 5′ end conjugates can inhibit 5′ to 3′ exonucleolytic cleavage (e.g., naproxen; ibuprofen; small alkyl chains; aryl groups; heterocyclic conjugates; modified sugars (D-ribose, deoxyribose, glucose etc.)).

In some embodiments, nucleic acid molecules encompassed by the present invention can include base modifications and/or substitutions of natural nucleobases.

The term “unmodified” or “natural” nucleobases include the purine bases adenine (A) and guanine (G), and the pyrimidine bases thymine (T), cytosine (C) and uracil (U). In some embodiments, nucleic acid molecules can comprise one or more nucleobase-modified nucleotides. It can comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, abut 27, about 28, about 29, or more nucleobase-modified nucleotides. In some examples, nucleic acid molecules can comprise about 1% to 10% modified nucleotides, or about 10% to 50% modified nucleotides. Modified bases refer to nucleotide bases such as, for example, adenine (A), guanine (G), cytosine (C), thymine (T), uracil (U), xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine, 5-propynyluridine, 5-propynylcytidine, 6-methyladenine, 6-methylguanine, N,N,-dimethyladenine, 2-propyladenine, 2-propylguanine, 2-aminoadenine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties can be, or be based on, mannoses, arabinoses, glucopyranoses, galactopyranoses, 4′-thioribose, and other sugars, heterocycles, or carbocycles.

Exemplary modified nucleobases include, but are not limited to, other synthetic and naturally modified nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. In some particular embodiments, nucleobase-modified nucleotides useful in the invention include, but are not limited to: 5-bromo-uridine, 5-iodo-uridine, 5-methyl-cytidine, ribo-thymidine, 2-aminopurine, 5-fluoro-cytidine, and 5-fluoro-uridine, 2,6-diaminopurine, 4-thio-uridine; and 5-amino-allyl-uridine and the like.

In some embodiments, nucleic acid molecules encompassed by the present invention can also contain nucleotides with base analogues.

The nucleobase can be naturally occurring non canon bases such as CpG islands, inosine which can base pair with C, U or A, thiouridine, dihydrouridine, queuosine, xanthine, hypoxanthine, nubularine, isoguanisine, tubercidine and wyosine. Other analogues can include fluorophores (e.g., rhodamine, fluorescein) and other fluorescent base analogues such as 2-AP (2-aminopurine), 3-MI, 6-MI, 6-MAP, pyrrolo-dC, modified and improved derivatives of pyrrolo-dC, furan-modified bases, and tricyclic cytosine family (e.g., 1,3-Diaza-2-oxophenothiazine, tC; oxo-homologue of tC, tC; 1,3-diaza-2-oxophenoxazine). Nucleobase modified nucleotides can also include universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group. As used herein, a universal nucleobase is any modified nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenzyl, tetracenyl, pentacenyl, and structural derivatives thereof. In some embodiments, the nucleotides of the nucleic acid molecules can incorporate base analogues and modified bases that are described in U.S. Pat. Nos. 6,008,334; 6,107,039; 6,664,058; 7,678,894; 7,786,292; and 7,956,171; U.S. Pat. Publ. Nos. 2013/122,506 and 2013/0296402; carboxamido-modified bases as described in PCT Pat. Publ. No. WO 2012/061810).

In some embodiments, modified nucleic acid molecules encompassed by the present invention can comprise artificial nucleic acid analogues.

The term “artificial nucleic acid analogues” or simply “nucleic acid analogues” refers to compounds that are structurally similar to naturally occurring DNA or RNA. An analogue can have any of the phosphate backbone, sugar, or the nucleobase (i.e., G, C, T, U, and A) altered. In some embodiments, the modified nucleotide can be an unlocked nucleomonomer agent (UNA). UNAs include any monomer unit suitable for inclusion in an oligomeric or polymeric composition such as an oligonucleotide or polynucleotide and which have, in reference to nucleosides or nucleotides, an unlocked or acyclic sugar moiety. Where such UNAs are included in a larger oligomer or polymer, such larger oligomer or polymer, e.g., oligonucleotide, can also be referred to as a UNA oligomer or UNA polymer, or UNA oligonucleotide. Where a UNA is included in a standard nucleotide, such variant nucleotide is referred to as a UNA nucleotide. Where a UNA is included in a standard nucleoside, such variant nucleoside is referred to as a UNA nucleoside. UNAs can be used as substitutes for nucleosides or nucleotides in oligonucleotides. In this case, UNAs, whether the monomer or oligomer containing the monomer, have often been referred to as “unlocked nucleic acids” in the art. When referred to as an unlocked nucleic acid herein, one of skill will understand that the inventors are referring to UNAs. According to the present invention, UNAs are not naturally occurring nucleomonomer agents. In one embodiment, one or more nucleotides in the nucleic acid molecule can be replaced with one or more unlocked nucleic acid/nuclomonomer agent (UNA) moieties, including those described in, e.g., PCT Publ. WO 2015/148580. A UNA oligomer can be a chain composed of UNA monomers, as well as various nucleotides that can be based on naturally-occurring nucleosides or modified nucleotides. UNA oligomers have been reported to have reduced off-target effects as compared to counterpart oligonucleotides lacking the modifications. Other UNA modifications and uses which can be utilized in accordance with the present invention include any of those disclosed in US Publication US20150232851, U.S. Pat. No. 9,051,570, US Publication US20150232849, EP Publication EP2162538, US Publication US20150239926, US Publication US20150239834, US Publication US20150141678, International publication WO2015074085, and/or EP Publication EP2370577.

In some embodiments, artificial nucleic acid analogues with backbone analogues include, but are not limited to, a bicyclic nucleotide analogue such as locked nucleic acid (LNA), bridged nucleic acid (BNA), glycol nucleic acid (GNA), threose nucleic acid (TNA), and morpholino. The modified oligonucleotides that comprise these backbone analogues, although having a different backbone sugar, or in case of PNA, an amino acid residue in place of the ribose phosphate, still bind to RNA or DNA according to Watson and Crick pairing, but are immune to nuclease activity. LNAs are described, for example, in U.S. Pat. Nos. 6,268,490; 6,316,198; 6,403,566; 6,770,748; 6,998,484; 6,670,461; and 7,034,133; PCT Publ. No. 99/14226. Other suitable locked nucleotides that can be incorporated in the nucleic acid molecules encompassed by the present invention include those described in U.S. Pat. Nos. 6,403,566; 6,833,361; and 7,060,809. Other locked nucleic acid derivatives, such as D-oxy-LNA, α-L-oxy-LNA, β-D-amino-LNA, α-L-amino-LNA, thio-LNA, α-L-thio-LNA, seleno-LNA, methylene-LNA and β-D-ENA, can be incorporated into nucleic acid molecules encompassed by the present invention. Those LNA derivatives described in U.S. Pat. Nos. 7,569,575; 8,084,458; and 8,429,390, can also be incorporated into the nucleic acid molecules.

In some embodiments, nucleic acid molecules encompassed by the present invention can comprise one or more sugar-modified nucleotides.

It can comprise about 1, about 2, about 3, about 4, about 5, about 6, about 7, about 8, about 9, about 10, or more sugar-modified nucleotides. Sugar-modified nucleotides useful in the invention include, but are not limited to: 2′-fluoro modified ribonucleotide, 2′-OMe modified ribonucleotide, 2′-deoxy ribonucleotide, 2′-amino modified ribonucleotide and 2′-thio modified ribonucleotide. The sugar-modified nucleotide can be, for example, 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine or 2′-amino-butyryl-pyrene-uridine. In addition to 2′ modification of the backbone sugar, the sugar group can be modified at other positions. The sugar group can comprise two different modifications at the same carbon of the sugar. The sugar group can also contain one or more carbons that possess the opposite stereochemical configuration than that of the corresponding carbon in ribose. Thus, a nucleic acid molecule can include nucleotides containing, e.g., arabinose, as the sugar. The nucleotide can have an alpha linkage at the 1′ position on the sugar, e.g., alpha-nucleosides. The nucleotide can also have the opposite configuration at the 4′-position, e.g., C5′ and H4′ or substituents replacing them are interchanged with each other. When the CS' and H4′ or substituents replacing them are interchanged with each other, the sugar is said to be modified at the 4′ position.

The nucleic acid molecules encompassed by the present invention can also include abasic sugars, which lack a nucleobase at C-1′ or have other chemical groups in place of a nucleobase at C1′ (see, e.g., U.S. Pat. No. 5,998,203). These abasic sugars can also be further containing modifications at one or more of the constituent sugar atoms. In other embodiments, nucleic acid molecules can also contain one or more sugars that are the L isomers. In one aspect, modification to the sugar group can also include replacement of the 4′-O with a sulfur, optionally substituted nitrogen or CH2 group. In another aspect, modifications to the sugar group can also include acyclic nucleotides, wherein a C—C bond between ribose carbons is absent and/or at least one of ribose carbons or oxygen are independently or in combination absent from the nucleotide. Such acyclic nucleotides have been disclosed in U.S. Pat. Nos. 5,047,533 and 7,737,273, and U.S. Pat. Publ. No. 20130130378. It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration. The term “xylose configuration”, as used herein, refers to the placement of a substituent on the C3′ of ribose in the same configuration as the 3′-OH is in the xylose sugar. The hydrogen attached to C4′ and/or C1′ of the sugar group can be replaced by substitutes as described for 2′ modification. In one example, nucleic acid molecules encompassed by the present invention can comprise 2′-fluoro modified ribonucleotide. Preferably, the 2′-fluoro ribonucleotides are in the sense and antisense strands. More preferably, the 2′-fluoro ribonucleotides are every uridine and cytidine.

In some embodiments, the internucleoside linkage groups of the nucleic acid molecules encompassed by the present invention are modified.

The internucleoside linkage modification can be within the sense strand, antisense strand, or within the sense and antisense strands. The term “internucleoside linkage group” is intended to mean a group capable of covalently coupling together two nucleobases, such as between DNA residues, between RNA residues, between DNA and RNA residues and nucleotide analogues, between two non-LNA residues, between a non-LNA residue and a LNA residue, and between two LNA residues, etc. The naturally standard linkage is the phosphodiester linkage (PO linkage), consisting of —O—P(O)₂—O— (from 5′ to 3′ end), wherein the deoxyribose/ribose sugars are joined at both the 3′-hydroxyl and 5′-hydroxyl groups to phosphate groups in ester links, also known as “phosphodiester” bonds/linker. The linker can be modified by the replacement of one or both linking oxygens (i.e., oxygens that link the phosphate to the nucleoside), with nitrogen (bridged phosphoroamidates), sulfur (bridged phosphorothioates) and carbon (bridged methylenephosphonates). In some embodiments, the phosphate linker moiety can be replaced by non-phosphorus containing linkers, e.g., dephospho-linkers. While not wishing to be bound by theory, it is believed that since the charged phosphodiester group is the reaction center in nucleolytic degradation, its replacement with neutral structural mimics should impart enhanced nuclease stability. Examples of moieties which can replace the phosphate linker include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S-C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts.

In some embodiments, the modification of the linkage further comprises at least one of the oxygen atoms of one phosphate which is replaced or modified. In some aspects, one or both of the non-linking phosphate oxygens on the phosphate linker can be modified or replaced. The modified phosphates can include, but are not limited to, phosphonocarboxylate (in which one of the non-linking oxygen atoms has been replaced/modified with a carboxylic acid)(e.g., phosphoacetate, phosphonoformic acid, phosphoramidate); phosphorothioate (—O—P(O,S)—O—, —O—P(S)₂—O—); methylphosphonate (—O—P(OCH3)—O—), and alkyl or aryl phosphonates. As discussed herein, one or more atoms of the linkage between two successive monomers in the siRNA molecules encompassed by the present invention are modified. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═, —CH₂—CH₂—O—, —NR^{H}—, CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H} CO—NR^{H}—, —NR^{H}—CS—NR^{H}—, —NR^{H} C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—O—NR^{H}—, —NR^{H}—CO—CH₂—, —O—CH₂—CO—NR^{H}—, —O—CH₂—CH₂—NR^{H}—, —CH═N—O—, —CH₂—NR^{H} O—, —CH₂—O—N═, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, —O—PO(NR^{H})—O—, —O—PO(OCH₂CH₂S—R)—O—, —O—PO(BH₃)—O—, —O—PO(NHR^{H})—, —O—P(O)₂—NR^{H}—, —NR^{H}—P(O)₂—O—, —NR″—CO—O—, —NR^{H}—CO—NR^{H}—, —O—CO—O—, —O—CO—NR^{H}—, —NR^{H}—CO—CH₂—, —O—CH₂—CO—NR^{H}—, —O—CH₂—CH₂—NR^{H}—, —CO—NR^{H}CH₂, —CH₂—NR^{H}—CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, —CH₂—NCH₃—O—CH₂—, —S—CH₂—CH═, —O-PO(OCH₂CH₃)—O—, —O—PO(OCH₂CH₂S—R)—O—, —O—PO(BH₃)—O—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O-P(O)₂—O—, —O—P(O,S)—O—, —O—P(S)₂—O—, —O—P(O,NR^{H})—O—, —O—PO(R″)—O—, —O—PO(CH₃)—O—, and —O—PO(N—R^{N})—O—, wherein R^{H} is selected from hydrogen and C1-alkyl.

In the context encompassed by the present invention, preferred examples include phosphate, phosphodiester (PO) linkages and phosphorothioate (PS) linkages. Phosphorodithioates have both non-bridging oxygens replaced by sulfur. The phosphorus center in the phosphorodithioates is achiral which precludes the formation of oligonucleotide diastereomers. Thus, while not wishing to be bound by theory, modifications to both non-linking oxygens, which eliminate the chiral center, e.g., phosphorodithioate formation, can be desirable in that they cannot produce diastereomer mixtures. Thus, the non-linking oxygens can be independently any one of O, S, Se, B, C, H, N, or OR (R is alkyl or aryl). In some embodiments, nucleic acid molecules encompassed by the present invention can contain one or more phosphorothioate linkages. For example, the polynucleotide can be partially phosphorothioate-linked, for example, phosphorothioate linkages can alternate with phosphodiester linkages. In certain embodiments, the oligonucleotide is fully phosphorothioate-linked. In other embodiments, the oligonucleotide has from one to seven, one to five or one to three phosphodiester linkages. Phosphorothioate linkages have been used to render oligonucleotides more resistant to nuclease cleavage. In addition to normal 5′-3′ linkage, modified oligonucleotide can have 5′-2′ linkage and those having inverted polarity wherein the adjacent pairs of nucleoside units are linked 3′-5′ to 5′-3′ or 2′-5′ to 5′-2′. Representative U.S. patents that teach modifications of internucleoside linkage groups include U.S. Pat. Nos. 5,519,126; 5,536,821; 5,541,306; 5,550,111; 5,563,253; 5,571,799; 5,587,361; 5,625,050; 5,378,825; 5,697,248 and 7,368,439. Other references that teach internucleoside linkage modifications include Mesmaeker et al. (1995) Curr. Opin. Siruct. Biol. 5:343-355; Freier and Altmann (1997) Nucl. Acids Res. 25:4429-4443; and Micklefield (2001) Curr. Med. Chem. 8:1157-1179.

In some embodiments, nucleic acid molecules encompassed by the present invention can comprise one or more backbone-modified nucleotides.

The backbone-modified nucleotide is within the sense strand, antisense strand, or within the sense and antisense strands. A normal “backbone”, as used herein, refers to the repeating alternating sugar-phosphate sequences in a DNA or RNA molecule. In naturally occurring DNA and RNA molecules, the backbone of a nucleic acid molecule includes deoxyribose/ribose sugars joined at both the 3′-hydroxyl and 5′-hydroxyl groups to phosphate groups in ester links (i.e. PO linkage). The natural phosphodiester bonds can be replaced by amide bonds but the four atoms between two sugar units are kept. Such amide modifications can increase the thermodynamic stability of duplex formed with miRNA complement (see, e.g., Mesmaeker et al. (1997) Pure Appl. Chem. 3:437-440). In some embodiments, nucleic acid molecules encompassed by the present invention can contain chemical modifications with respect to non-locked nucleotides in the sequence, such as 2′ modification with respect to 2′hydroxyl. For example, incorporation of 2′-position modified nucleotides in an siRNA molecule can increase both resistance of the oligonucleotides to nucleases and their thermal stability with complementary targets. Various modifications at the 2′ positions can be independently selected from those that provide increased nuclease resistance, without compromising molecular interactions with the target or cellular machinery. Such modifications can be selected on the basis of their increased potency in vitro or in vivo. In some embodiments, the 2′ modification can be independently selected from a number of different “oxy” or “deoxy” substituents. Examples of “oxy”-2′ hydroxyl group modifications include alkoxy or aryloxy (e.g., Omethyl, R═H, alkyl, cycloalkyl, aryl, aralkyl, heteroaryl or sugar; polyethyleneglycols (PEG), O(CH₂CH₂O)ₙCH₂CH₂OR (n=1-50); O-AMINE or O—(CH₂)ₙAMINE (n=1-10), AMINE=NH₂; alkylamino, dialkylamino, heterocyclyl, arylamino, diaryl amino, heteroaryl amino, diheteroaryl amino, ethylene diamine or poyamino; and O—CH₂CH₂(NCH₂CH₂NMe₂)₂). “Deoxy” modifications include hydrogen (i.e., deoxyribose sugars, which are of particular relevance to the single-strand overhangs); halo (e.g., fluoro); amino (e.g., NH₂; alkylamino, dialkylamino, heterocyclyl, arylamino, diaryl amino, heteroaryl amino, diheteroaryl amino, or amino acid; NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino; —NHC(O)R(R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl.

Substantially all, or all, nucleotide 2′ positions of the non-locked nucleotides can be modified in certain embodiments. For example, the 2′ modifications can each be independently selected from O-methyl and fluoro. In exemplary embodiments, purine nucleotides each have a 2′O-methyl and pyrrolidine nucleotides each have a 2′-F. In accordance with the present invention, 2′ position modifications can also include small hydrocarbon substituents. The hydrocarbon substituents include alkyl, alkenyl, alkynyl, and alkoxyalkyl, where the alkyl (including the alkyl portion of alkoxy), alkyl and alkyl can be substituted or unsubstituted. The alkyl, alkenyl, and alkynyl can be C1 to C10 alkyl, alkenyl or alkynyl, such as C1, C2, or C3. The hydrocarbon substituents can include one or two or three non-carbon atoms, which can be independently selected from N, O, and/or S. The 2′ modifications can further include the alkyl, alkenyl, and alkynyl as O-alkyl, O-alkenyl, and O-alkynyl. Exemplary 2′ modifications in accordance with the invention include 2′-H, 2′-O-alkyl (C1-3alkyl, such as 2′O-Methyl or 2′OEt), 2′-O-methoxyethyl (2′-0-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethyiaminoethyioxyethyl (2′-O-DMAEOE), 2′-O—N-methylacetamido (2′-O-NMA) or gem 2′-OMe/2′F substitutions. In some embodiments, nucleic acid molecules encompassed by the present invention contains at least one 2′ position modified as 2′O-Methoxy (2′-OMe) in non-locked nucleotides. The oligonucleotide can contain from 1 to about 5 2′—O-Methoxy (2′-OMe) modified nucleotides, or from 1 to about 3 2′-O-Methoxy (2′-OMe) modified nucleotides. In some embodiments, all the nucleotides of the miR-124 mimic contain 2′-O-Methoxy (2′-OMe) modification. Other exemplary combinations of different types of 2′ position modifications can contain at least one 2′-halo modification (e.g., in place of a 2′ hydroxyl), such as 2′-fluoro, 2′-chloro, 2′-bromo, and 2′-iodo.

In some embodiments, the backbone of a strand or the strand of the nucleic acid molecule can be constructed wherein the phosphate linker and ribose sugar are replaced by nuclease resistant nucleosides or nucleotide surrogates. While not wishing to be bound by theory, it is believed that the absence of a repetitively charged backbone diminishes binding to proteins that recognize polyanions (e.g., nucleases). As non-limiting examples, such nucleotide surrogates include morpholino, cyclobutyl, pyrrolidine, peptide nucleic acid (PNA), aminoethylglycyl PNA (Aegina) and backbone-extended pyrimidine PNA (bepPNA) nucleoside surrogates (e.g., U.S. Pat. Nos. 5,359,044; 5,519,134; 5,142,047 and 5,235,033; Bioorganic & Medicinal Chemistry (1996), 4:5-23). A surrogate for the replacement of the sugar-phosphate backbone involves a PNA surrogate (peptide nucleic acid). The term “peptide nucleic acid (PNA)” is chemically synthesized polymer similar to DNA and RNA, wherein the backbone is composed of repeating N-(2-aminoethyl)-glycine (AEG) units linked by peptide bonds (Nielsen et al. (1991) Science 254:1497-1500). Synthetic oligonucleotides with PNAs have higher binding strength and greater specificity in binding to complementary DNAs or RNAs, with a PNA/DNA base mismatch being more desirable than a similar DNA/RNA duplex. PNAs are not easily recognized by either nucleases or proteases, making them resistant to enzyme degradation. PNAs are also stable over a wide pH range. PNA has been suggested for use in antisense and anti-gene therapy in a number of studies. PNA is resistant to DNases and proteases and can be further modified for increased cell penetration, etc.

PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup et al. (1996) Bioorg. Med. Chem. 4:5-23; or as probes or primers for DNA sequence and hybridization (Perry-O'Keefe et al. (1996) Proc. Natl. Acad. Sci. U.S.A. 93:14670-14675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup et al. (1996) Bioorg. Med. Chem. 4:5-23). The synthesis of PNA-DNA chimeras can be performed as described in Hyrup et al. (1996) Bioorg. Med. Chem. 4:5-23 and Finn et al. (1996) Nucleic Acids Res. 24:3357-3363. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-5988). PNA monomers are then coupled in a step-wise manner to produce a chimeric molecule with a 5′ PNA segment and a 3′ DNA segment (Finn et al. (1996) Nucleic Acids Res. 24:3357-3363). Alternatively, chimeric molecules can be synthesized with a 5′ DNA segment and a 3′ PNA segment (Peterser et al. (1975) Bioorganic Med. Chem. Lett. 5:1119-11124).

Nucleic acid molecules encompassed by the present invention can also contain additional modifications, such as mismatches, bulges, or crosslinks. Similarly, they can also include other conjugates, such as linkers, heterofunctional cross linkers, dendrimer, nano-particle, peptides, organic compounds (e.g., fluorescent dyes), and/or photocleavable compounds. In some embodiments, nucleic acid molecules encompassed by the present invention can comprise any combination of two or more modifications as described herein. The nucleic acid sequences can comprise, independently, one or more modifications to one or more sugar moieties, to one or more intemucleoside linkages, and/or to one or more nucleobases. As disclosed herein, these sequences can be modified with any combinations of chemical modifications.

In some embodiments, the nucleic acid molecule is a siRNA which comprises a nucleic acid sequence wherein the sense strand and anti-sense strand comprise one or more mismatches, for example, 1, 2, 3, 4, 5, 6, 7, 8, 9, 10, or more mismatches. The term “mismatch” refers to a basepair consisting of non-complementary bases, e.g., not normal complementary G:C, A:T or A:U base pairs. In some embodiments, the antisense strand of the siRNA molecule encompassed by the present invention and the target mRNA sequence can comprise one or more mismatches, for example, 1, 2, 3, 4, 5, 6, 7, 8, 9, 10, or more mismatches. In some instances, the mismatch can be downstream of the cleavage site referencing the antisense strand. More preferably, the mismatch can be present within 1-6 nucleotides from the 3′ end of the antisense strand. In another embodiment, the siRNA molecule encompassed by the present invention comprises a bulge, e.g., one or more, e.g., 1, 2, 3, 4, 5, 6, 7, 8, 9, 10, or more, unpaired bases in the duplex siRNA. Preferably, the bulge can be in the sense strand.

In some embodiments, the siRNA molecule encompassed by the present invention comprises one or more (e.g., about 1, 2, 3, 4, 5, 6, 7, 8, 9, 10, or more) crosslinks, e.g., a crosslink wherein the sense strand is crosslinked to the antisense strand of the siRNA duplex. Crosslinkers useful in the invention are those commonly known in the art, including, but not limited to, psoralen, mitomycin C, cisplatin, chloroethylnitrosoureas and the like. Preferably, the crosslink is present downstream of the cleavage site referencing the antisense strand, and more preferably, the crosslink is present at the 5′ end of the sense strand. In accordance with the present invention, siRNA derivatives are also included, such as a siRNA derivative having a single crosslink (e.g., a psoralen crosslink), a siRNA having a photocleavable biotin (e.g., photocleavable biotin), a peptide (e.g., a Tat peptide), a nanoparticle, a peptidomimetic, organic compounds (e.g., a dye such as a fluorescent dye), or dendrimer.

In some embodiments, nucleic acid molecules encompassed by the present invention can include other appended groups, such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Pat. Publ. No. WO 88/09810) or the blood-brain barrier (see, e.g., PCT Publ. No. WO 89/10134). In addition, nucleic acid molecules can be modified with hybridization-triggered cleavage agents (see, e.g., Krol et al. (1988) BioTechniques 6:958-976) or intercalating agents (see, e.g., Zon (1988) Pharm. Res. 5:539-549).

k. Vectors and Other Nucleic Acid Vehicles

In accordance with the present invention, nucleic acid molecules and variants thereof can be produced by any methods known in the art, such as direct synthesis and genetic recombination techniques. Nucleic acid molecules can be present in any forms such as pure nucleic acid molecules, plasmids, DNA vectors, RNA vectors, viral vectors and particles. The term “vector” refers to a nucleic acid molecule capable of transporting another nucleic acid to which it has been linked. Vectors encompassed by the present invention can also be used to deliver the packaged polynucleotides to a cell, a local tissue site or a subject.

One type of vector is a “plasmid,” which refers to a circular double-stranded DNA loop into which additional nucleic acid segments can be ligated. Another type of vector is a “viral vector,” wherein additional DNA segments can be ligated into a viral genome. Viral nucleic acid delivery vectors can be of any kind, including Retroviruses, Adenoviruses, Adeno-associated viruses, Herpes simplex viruses and variants thereof. Viral vector technology is well-known and described in Sambrook et al. (2012, Molecular Cloning: A Laboratory Manual, Cold Spring Harbor Laboratory (4^{th} Ed.), New York).

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

Recombinant expression vectors encompassed by the present invention comprise a nucleic acid encompassed by the present invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Fzymology: Gene Fxpression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors encompassed by the present invention can be introduced into host cells to thereby produce proteins or peptides, including fusion proteins or peptides, encoded by nucleic acids as described herein. For example, in general, vectors contain an origin of replication functional in at least one organism, a promoter sequence and convenient restriction endonuclease site, and one or more selectable markers e.g., a drug resistance gene. Vectors can comprise native or non-native promoters operably linked to the polynucleotides encompassed by the present invention. The promoters selected can be strong, weak, constitutive, inducible, tissue specific, development stage-specific, and/or organism specific. In some embodiments, the vector can comprise regulatory sequences, such as, enhancers, transcription and translation initiation and termination codons, which are specific to the type of host cell into which the vector is to be introduced.

Recombinant expression vectors for use according to the present invention can be designed for expression of a polypeptide corresponding to a biomarker encompassed by the present invention in prokaryotic (e.g., E. coli) or eukaryotic cells (e.g., insect cells, such as using baculovirus expression vectors, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E col with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.), which fuse glutathione S-transferase (GST), maltose E binding protein, or protein A, respectively, to the target recombinant protein.

Representative, non-limiting examples of suitable inducible non-fusion E. coli expression vectors include pTrc (Amann et al. (1988) Gene 69:301-315) and pET 11d (Studier et al. (1991) Meth. Enzvmol. 185:60-89). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman (1990) Meth. Enzymol. 185:119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences encompassed by the present invention can be carried out by standard DNA synthesis techniques.

In some embodiments, the expression vector is a yeast expression vector. Examples of vectors for expression in yeast S. cerevisiae include pYepSecl (Baldari et al. (1987) FMBO J. 6:229-234), pMFa (Kuran and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In some embodiments, a nucleic acid encompassed by the present invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. U.S.A. 86:5473-5477), pancreas-specific promoters (Edlund el al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Camper and Tilghman (1989) Genes Dev. 3:537-546).

The present invention also provides recombinant expression vectors for expressing antisense nuceleic acids, as described further below. For example, DNA molecule can be operably linked to a regulatory sequence in a manner which allows for expression (by transcription of the DNA molecule) of an RNA molecule which is antisense to the mRNA encoding a polypeptide encompassed by the present invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al. (1986) Trends Genet. 1(1)).

In some embodiments, a retroviral vector is useful according to the present invention. Retroviruses are named because reverse transcription of viral RNA genomes to DNA is required before integration into the host cell genome. As such, the most important features of retroviral vectors are the permanent integration of their genetic material into the genome of a target/host cell. The most commonly used retroviral vectors for nucleic acid delivery are lentiviral vehicles/particles. Some examples of lentiviruses include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia virus, visna-maedi and caprine arthritis encephalitis virus (CAEV).

Typically, lentiviral particles making up the gene delivery vehicle are replication defective on their own, such that they are unable to replicate in the host cell and can infect only one cell (also referred to as “self-inactivating”). Lentiviruses are able to infect both dividing and non-dividing cells by virtue of the entry mechanism through the intact host nuclear envelope (Naldini et al. (1998) Curr. Opin. Biotechnol. 9:457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells. The term “recombinant” refers to a vector or other nucleic acid containing both lentiviral sequences and non-lentiviral retroviral sequences. Lentiviral particles can be generated by co-expressing the virus packaging elements and the vector genome itself in a producer cell such as HEK293T cells, 293G cells, STAR cells, and other viral expression cell lines. These elements are usually provided in three (in second generation lentiviral systems) or four separate plasmids (in third generation lentiviral systems). The producer cells are co-transfected with plasmids that encode lentiviral components including the core (i.e., structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector).

The envelope proteins of recombinant lentiviral vectors can be heterologous envelope proteins from other viruses, such as the G protein of vesicular stomatitis virus (VSV G) or baculoviral gp64 envelop proteins. The VSV-G glycoprotein can especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pikefry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedrovirus (AcMNPV), Anagrapha falcifera nuclear polyhedrosis virus, Bombyr mori nuclear polyhedrosis virus, Choristoneura fumiferana nucleopolyhedrovirus, Orgyia pseudotsugata single capsid nuclear polyhedrosis virus, Epiphyas postvittana nucleopolyhedrovirus, Hyphantria cunea nucleopolyhedrovirus, Galleria mellonella nuclear polyhedrosis virus, Dhori virus, Thogoto virus, Antheraea pemyi nucleopolyhedrovirus or Batken virus.

Methods for generating recombinant lentiviral particles are discussed in the art, for example, U.S. Pat. Nos. 8,846,385; 7,745,179; 7,629,153; 7,575,924; 7,179,903; and 6,808,905.

Lentivirus vectors used can be selected from, but are not limited to pLVX, pLenti, pLenti6, pLJMI, FUGW, pWPXL, pWPI, pLenti CMV puro DEST, pLJM1-EGFP, pULTRA, pInducer20, pHIV-EGFP, pCW57.1, pTRPE, pELPS, pRRL, and pLionII. Lentiviral vehicles known in the art can also be used (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994,136; PCT Publ. No. WO 2012079000).

Additional elements can be included in recombinant lentiviral particles including, retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer. The effector module is linked to the vector. In addition to lentiviral vectors based on complex HIV-1/2, retroviral vectors based on simple gamma-retroviruses have been widely used to deliver therapeutic nucleic acids and demonstrated clinically as one of the most efficient and powerful nucleic acid delivery systems capable of transducing a broad range of cell types. Example species of gamma retroviruses include the murine leukemia viruses (MLVs) and the feline leukemia viruses (FeLV). Gamma-retroviral vectors derived from a mammalian gamma-retrovirus such as murine leukemia viruses (MLVs) can be recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xpr1) receptor, but differ in their species tropism. Xenotropic viruses such as NZB-9-1 infect human and other species but not murine species, whereas polytropic viruses such as focus-forming viruses (MCF) infect murine, human and other species.

Gamma-retroviral vectors can be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the compositions encompassed by the present invention that is to be packaged in newly formed viral particles. The recombinant gamma-retroviral vectors can be pseudotyped with envelope proteins from other viruses. Envelope glycoproteins are incorporated in the outer lipid layer of the viral particles which can increase/alter the cell tropism. Exemplary envelop proteins include the gibbon ape leukemia virus envelope protein (GALV) or vesicular stomatitis virus G protein (VSV-G), or Simian endogenous retrovirus envelop protein, or Measles Virus H and F proteins, or Human immunodeficiency virus gp120 envelope protein, or cocal vesiculovirus envelop protein (see, e.g., U.S. Publ. No. 2012/164118). In other embodiments, envelope glycoproteins can be genetically modified to incorporate targeting/binding ligands into gamma-retroviral vectors, binding ligands including, but not limited to, peptide ligands, single chain antibodies and growth factors (Waehler et al. (2007) Nat. Rev. Genet. 8:573-587). These engineered glycoproteins can retarget vectors to cells expressing their corresponding target moieties. In other aspects, a “molecular bridge” can be introduced to direct vectors to specific cells. The molecular bridge has dual specificities: one end can recognize viral glycoproteins, and the other end can bind to the molecular determinant on the target cell. Such molecular bridges, such as ligand-receptor, avidin-biotin, chemical conjugations, monoclonal antibodies, and engineered fusogenic proteins, can direct the attachment of viral vectors to target cells for transduction (Yang et al. (2008) Biotechnol. Bioeng. 101:357-368; Maetzig et al. (2011) Viruses 3:677-713). The recombinant gamma-retroviral vectors can be self-inactivating (SIN) gammaretroviral vectors. The vectors are replication incompetent. SIN vectors can harbor a deletion within the 3′ U3 region initially comprising enhancer/promoter activity. Furthermore, the 5′ U3 region can be replaced with strong promoters (needed in the packaging cell line) derived from cytomegalovirus or RSV, or an internal promoter of choice, and/or an enhancer element. The choice of the internal promoters can be made according to specific requirements of gene expression needed for a particular purpose encompassed by the present invention.

Similarly, recombinant adeno-associated viral (rAAV) vectors can be used to package and deliver nucleic acid molecules encompassed by the present invention. Such vectors or viral particles can be designed to utilize any of the known serotype capsids or combinations of serotype capsids. The serotype capsids can include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10 (see, for example. U.S. Pat. Publ. 20030138772) or variants thereof. AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors can be manufactured by standard methods in the art such as by triple transfection, in sf9 insect cells or in suspension cell cultures of human cells such as HEK293 cells. Nucleic acid molecules encompassed by the present invention can be encoded in one or more viral genomes to be packaged in the AAV capsids. Such vectors or viral genomes can also include, in addition to at least one or two ITRs (inverted terminal repeats), certain regulatory elements necessary for expression from the vector or viral genome. Such regulatory elements are well-known in the art and include for example promoters, introns, spacers, stuffer sequences, and the like.

In addition, non-viral delivery systems of nucleic acid molecules are well-known in the art. The term “non-viral vectors” collectively refers to any vehicles that transfer nucleic acid molecules encompassed by the present invention into cells of interest without using viral particles. Representative examples of such non-viral delivery vectors are vectors that coat nucleic acids based on the electrical interaction between cationic sites on the vectors and anionic sites on the negatively charged nucleic acids constituting genes. Some exemplary non-viral vectors for delivery can include naked nucleic acid delivery systems, polymeric delivery systems and liposomal delivery systems. Cationic polymers and cationic lipids are used for nucleic acids delivery because they can easily complex with the anionic nucleotides. Commonly used polymers can include, but are not limited to, polyethylenimine, poly-L-lysin, chitosans, and dendrimers. Cationic lipids can include but are not limited to, monovalent cationic lipids, polyvalent cationic lipids, guanidine containing lipids, cholesterol derivative compounds, cationic polymers: Poly(ethylenimine) (PEI), poly-1-lysine) (PLL), protamine, other cationic polymers and lipid-polymer hybrid.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells can integrate the foreign DNA into their genome. In order to identify and select these integrants, a gene that encodes a selectable marker (e.g., for resistance to antibiotics like neo, DHFR, Gln synthetase, ADA, and the like) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

Accordingly, the present invention encompasses host cells into which a recombinant expression vector encompassed by the present invention has been introduced. The terms “host cell” and “recombinant host cell” are used interchangeably herein. It is understood that such terms refer not only to the particular subject cell but to the progeny or potential progeny of such a cell. Because certain modifications can occur in succeeding generations due to either mutation or environmental influences, such progeny can not, in fact, be identical to the parent cell, but are still included within the scope of the term as used herein. A host cell can be any prokaryotic (e.g., E. coli) or eukaryotic cell (e.g., insect cells, yeast or mammalian cells).

2. Protein Agents

Another aspect encompassed by the present invention involves the use of amino acid-based agents. The agents can include, but are not limited to, antibodies, fusion proteins, synthetic polypeptides, and peptides, as well as fragments thereof (e.g., biologically active fragments). Polynucleotides that encode such amino acid-based compounds are also provided.

Amino acid-based agents (e.g., antibodies and recombinant proteins) of the present invention can exist as a whole polypeptide, a plurality of polypeptides or fragments of polypeptides, which independently can be encoded by one or more nucleic acids, a plurality of nucleic acids, fragments of nucleic acids or variants of any of the aforementioned.

The term “polypeptide” refers to a polymer of amino acid residues (natural or unnatural) linked together most often by peptide bonds. The term, as used herein, refers to proteins, polypeptides, and peptides of any size, structure, or function. Thus, the term polypeptide is mutually inclusive of the terms “peptide” and “protein.” The term “fusion protein” refers to a fusion polypeptide molecule comprising at least two amino acid sequences from different resources, wherein the component amino acid sequences are linked to each other by peptide-bonds, either directly or through one or more peptide linkers. In some instances the polypeptide encoded is smaller than about 50 amino acids and the polypeptide is then termed a “peptide.” If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide can be a single molecule or can be a multi-molecular complex such as a dimer, trimer or tetramer. They can also comprise single chain or multichain polypeptides and can be associated or linked. The term polypeptide can also apply to amino acid polymers in which one or more amino acid residues are an artificial chemical analogue of a corresponding naturally occurring amino acid.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

In some embodiments, the native polypeptide corresponding to a marker can be isolated from cells or tissue sources by an appropriate purification scheme using standard protein purification techniques. In another embodiment, polypeptides corresponding to a marker encompassed by the present invention are produced by recombinant DNA techniques. Alternative to recombinant expression, a polypeptide corresponding to a marker encompassed by the present invention can be synthesized chemically using standard peptide synthesis techniques.

Polypeptide fragments include polypeptides comprising amino acid sequences sufficiently identical to or derived from an amino acid sequence of interest, but which includes fewer amino acids than the full length protein. They can also exhibit at least one activity of the corresponding full-length protein. Typically, biologically active portions comprise a domain or motif with at least one activity of the corresponding protein. A biologically active portion of a protein encompassed by the present invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide encompassed by the present invention.

Preferred polypeptides have an amino acid sequence of a polypeptide of interest, such as a polypeptide encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

The term “identity” as is applies to amino acid sequences is defined as the percentage of residues in the candidate amino acid sequence that are identical with the residues in the amino acid sequence of a second sequence after aligning the sequences and introducing gaps, if necessary, to achieve the maximum percent identity. Methods and computer programs for alignment are well-known in the art. It is understood that homology depends on a calculation of percent identity but can differ in value due to gaps and penalties introduced in the calculation.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. U.S.A. 87:2264-2268, modified as in Karlin and Altschul (1993) Proc. Natl. Acad. Sci. U.S.A. 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules encompassed by the present invention. BLAST protein searches can be performed with the XBLAST program, score=50, wordlength=3 to obtain amino acid sequences homologous to a protein molecules encompassed by the present invention. To obtain gapped alignments for comparison purposes, Gapped BLAST can be utilized as described in Altschul et a. (1997) Nucl. Aci Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used (see, for example, ncbi.nlm.nih.gov). Another preferred, non-limiting example of a mathematical algorithm utilized for the comparison of sequences is the algorithm of Myers and Miller (1988) Comput. Appl. Biosci. 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad Sci. U.S.A. 85:2444-2448. When using the FASTA algorithm for comparing nucleotide or amino acid sequences, a PAM120 weight residue table can, for example, be used with a k-tuple value of 2. The percent identity between two sequences can be determined using techniques similar to those described above, with or without allowing gaps. In calculating percent identity, only exact matches are counted.

The term “polypeptide variant” or “amino acid sequence variant” refers to molecules which differ in their amino acid sequence from a native or reference sequence. The amino acid sequence variants can possess substitutions, deletions, and/or insertions at certain positions within the amino acid sequence, as compared to a native or reference sequence. The terms “native” or “reference” when referring to sequences are relative terms referring to an original molecule against which a comparison can be made. Native or reference sequences should not be confused with wild type sequences. Native sequences or molecules can represent the wild-type (that sequence found in nature) but do not have to be identical to the wild-type sequence. Variants can possess at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5% or at least about 99.9% amino acid sequence identity (homology) to a native or reference sequence.

Polypeptide variants have an altered amino acid sequence and, in some embodiments, can function as either agonists or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein which function as either agonists or as antagonists can be identified by screening combinatorial libraries of mutants, e.g., truncation mutants, of the protein encompassed by the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides encompassed by the present invention from a degenerate oligonucleotide sequence. Methods for synthesizing degenerate oligonucleotides are known in the art (see, e.g. Narang (1983) Tetrahedron 39:3; Itakura et al. (1984) Annu. Rev. Biochem. 53:323; Itakura et al. (1984) Science 198:1056; and Ike et al. (1983) Nucleic Acid Res. 11:477).

In addition, libraries of fragments of the coding sequence of a polypeptide corresponding to a marker encompassed by the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein encompassed by the present invention (Arkin and Yourvan (1992) Proc. Natl. Acad. Sci. U.S.A. 89:7811-7815 and Delgrave et al. (1993) Pro. Engin. 6:327-331).

In some embodiments, “variant mimics” are provided. As used herein, the term “variant mimic” refers to a variant which contains one or more amino acids which would mimic an activated sequence. For example, glutamate can serve as a mimic for phospho-threonine and/or phospho-serine. Alternatively, variant mimics can result in deactivation or in an inactivated product containing the mimic, e.g., phenylalanine can act as an inactivating substitution for tyrosine; or alanine can act as an inactivating substitution for serine. The amino acid sequences can comprise naturally occurring amino acids and as such can be considered to be proteins, peptides, polypeptides, or fragments thereof. Alternatively, the agents encompassed by the present invention can comprise both naturally and non-naturally occurring amino acids. Non-naturally occurring amino acids can include, but are not limited to, amino acids comprising a carbonyl group, or an aminooxy group or a hydrazide group, or a semicarbazide group, or an azide group.

The term “homolog” as it applies to amino acid sequences is meant the corresponding sequence of other species having substantial identity to a second sequence of a second species.

The term “analog” is meant to include polypeptide variants which differ by one or more amino acid alterations, e.g., substitutions, additions or deletions of amino acid residues that still maintain the properties of the parent polypeptide.

The term “derivative” is used synonymously with the term “variant” and refers to a molecule that has been modified or changed in any way relative to a reference molecule or starting molecule. The present invention contemplates several types of compounds and/or compositions which are amino acid based including variants and derivatives. These include substitutional, insertional, deletional and covalent variants and derivatives. As such, included within the scope of the present invention is agents comprising substitutions, insertions, additions, deletions and/or covalent modifications. Amino acid residues located at the carboxy- and amino-terminal regions of the amino acid sequence of a peptide or protein can optionally be deleted providing for truncated sequences. Certain amino acids (e.g., C-terminal or N-terminal residues) can alternatively be deleted depending on the use of the sequence, as for example, expression of the sequence as part of a larger sequence which is soluble, or linked to a solid support.

“Substitutional variants” when referring to proteins are those that have at least one amino acid residue in a native or reference sequence removed and a different amino acid inserted in its place at the same position. The substitutions can be single, where only one amino acid in the molecule has been substituted, or they can be multiple, where two or more amino acids have been substituted in the same molecule. In one example, an amino acid in a polypeptide encompassed by the present invention is substituted with another amino acid having similar structural and/or chemical properties, e.g., conservative amino acid substitution. As used herein, the term “conservative amino acid substitution” refers to the substitution of an amino acid that is normally present in the sequence with a different amino acid of similar size, charge, polarity, solubility, hydrophobicity, hydrophilicity, and/or the amphipathic nature of the residues involved. Examples of conservative substitutions include the substitution of a non-polar (hydrophobic) residue such as alanine, proline, phenylalanine, tryptophan, isoleucine, valine, leucine and methionine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue, such as lysine, arginine or histidine for another, or the substitution of one acidic residue such as aspartic acid or glutamic acid for another acidic residue are additional examples of conservative substitutions. “Non-conservative substitutions” entail exchanging a member of one of these classes for another class. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue. Amino acid substitutions can be generated using genetic or chemical methods well-known in the art. Genetic methods can include site-directed mutagenesis, PCR, gene synthesis and the like. It is contemplated that methods of altering the side chain group of an amino acid by methods other than genetic engineering, such as chemical modification, can also be useful.

The term “insertional variants” when referring to proteins are those with one or more amino acids inserted immediately adjacent to an amino acid at a particular position in a native or starting sequence. As used herein, the term “immediately adjacent” refers to an adjacent amino acid that is connected to either the alpha-carboxy or alpha-amino functional group of a starting or reference amino acid. By contrast, the term “deletional variants” when referring to proteins, are those with one or more amino acids in the native or starting amino acid sequence removed. Ordinarily, deletional variants will have one or more amino acids deleted in a particular region of the molecule.

The term “derivatives” includes variants of a native or reference protein comprising one or more modifications with organic proteinaceous or non-proteinaceous derivatizing agents, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues can be present in the proteins used in accordance with the present invention. Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

In some embodiments, covalently modified polypetides (e.g., fusion proteins) are provided, such as polypeptides modified with a heterologous polypeptide and/or a non-polypeptide modification. For example, covalent derivatives specifically include fusion molecules in which proteins encompassed by the present invention are covalently bonded to a non-proteinaceous polymer. The non-proteinaceous polymer ordinarily is a hydrophilic synthetic polymer (i.e., a polymer not otherwise found in nature). However, polymers which exist in nature and are produced by recombinant or in vitro methods are useful, as are polymers which are isolated from nature. Hydrophilic polyvinyl polymers fall within the scope of this invention, e.g., polyvinylalcohol and polyvinylpyrrolidone. Particularly useful are polyvinylalkylene ethers such a polyethylene glycol, polypropylene glycol (PEG). The proteins can be linked to various non-proteinaceous polymers, such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337. Fusion molecules can further comprise proteins encompassed by the present invention which are covalently bonded to other biologically active molecules, or linkers.

The terms “chimeric protein” or “fusion protein” refer to polypeptides comprising all or part (preferably a biologically active part) of a polypeptide corresponding to a polypeptide encompassed by the present invention operably linked to a heterologous polypeptide (e.g., a polypeptide other than the biomarker polypeptide). Within the fusion protein, the term “operably linked” is intended to indicate that the polypeptide encompassed by the present invention and the heterologous polypeptide are fused in-frame to each other. The heterologous polypeptide can be fused to the amino-terminus or the carboxyl-terminus of the polypeptide encompassed by the present invention.

One useful fusion protein is a GST fusion protein in which a polypeptide corresponding to a marker encompassed by the present invention is fused to the carboxyl terminus of GST sequences. Such fusion proteins can facilitate the purification of a recombinant polypeptide encompassed by the present invention. In another embodiment, the fusion protein contains a heterologous signal sequence, immunoglobulin fusion protein, toxin, or other useful protein sequence. Chimeric and fusion proteins encompassed by the present invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide encompassed by the present invention can be cloned into such an expression vector such that the fusion moiety is linked in-frame to the polypeptide encompassed by the present invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present invention encompasses the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The term “features” when referring to proteins are defined as distinct amino acid sequence-based components of a molecule. Features of the proteins encompassed by the present invention include surface manifestations, local conformational shape, folds, loops, half-loops, domains, half-domains, sites, termini or any combination thereof. For example, the term “surface manifestation” when referring to proteins refers to a polypeptide based component of a protein appearing on an outermost surface. The term “local conformational shape” when referring to proteins refers to a polypeptide based structural manifestation of a protein which is located within a definable space of the protein. The term “fold” when referring to proteins refers to the resultant conformation of an amino acid sequence upon energy minimization. A fold can occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like. The term “turn” as it relates to protein conformation refers to a bend which alters the direction of the backbone of a peptide or polypeptide and can involve one, two, three or more amino acid residues. The term “loop” as it relates to proteins refers to a structural feature of a peptide or polypeptide which reverses the direction of the backbone of a peptide or polypeptide and comprises four or more amino acid residues (Oliva et al. (1997) J. Mol. Biol. 266:814-830). The term “half-loop” when referring to proteins refers to a portion of an identified loop having at least half the number of amino acid resides as the loop from which it is derived. It is understood that loops do not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4). The term “domain” when referring to proteins refers to a motif of a polypeptide having one or more identifiable structural or functional characteristics or properties (e.g., binding capacity and/or serving as a site for protein-protein interactions). The term “half-domain” when referring to proteins refers to a portion of an identified domain having at least half the number of amino acid resides as the domain from which it is derived. It is understood that domains do not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains can be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids can fold structurally to produce a domain, half-domain or subdomain). The term “site” as it pertains to amino acid-based embodiments is used synonymously with “amino acid residue” and “amino acid side chain.” A site represents a position within a peptide or polypeptide that can be modified, manipulated, altered, derivatized or varied within the amino acid based molecules encompassed by the present invention. The terms “termini” or “terminus” when referring to proteins refer to an extremity of a peptide or polypeptide. Such extremities are not limited only to the first or final site of the peptide or polypeptide but can include additional amino acids in the terminal regions. The polypeptide based molecules encompassed by the present invention can be characterized as having both an N-terminus (i.e., terminated by an amino acid with a free amino group (NH2)) and a C-terminus (i.e., terminated by an amino acid with a free carboxyl group (COOH)). Proteins encompassed by the present invention are in some cases made up of multiple polypeptide chains brought together by disulfide bonds or by non-covalent forces, such as multimers or oligomers. These proteins have multiple N- and C-termini. Alternatively, the termini of the polypeptides can be modified such that they begin or end, as the case can be, with a non-polypeptide based moiety such as an organic conjugate.

Once any of the features have been identified or defined as a component of a molecule encompassed by the present invention, any of several manipulations and/or modifications of these features can be performed by moving, swapping, inverting, deleting, randomizing or duplicating. Furthermore, it is understood that manipulation of features can result in the same outcome as a modification to the molecules encompassed by the present invention. For example, a manipulation which involved deleting a domain would result in the alteration of the length of a molecule just as modification of a nucleic acid to encode less than a full length molecule would. Modifications and manipulations can be accomplished by methods known in the art such as site directed mutagenesis.

In some embodiments, agents described herein can comprise one or more atoms that are isotopes. As used herein, the term “isotope” refers to a chemical element that has one or more additional neutrons, such as deuterium isotopes.

3. Antibody Agents

In another aspect, antibody agents, and variant and/or antigen-binding fragments thereof, are encompassed by the present invention.

a. Antibody Compositions

The term “antibody” or “Ab” is used in the broadest sense and specifically includes, without limitation, whole antibodies, monoclonal antibodies, polyclonal antibodies, multispecific antibodies (e.g., bispecific antibodies formed from at least two intact antibodies, trispecific, or antibodies of greater multispecificity), antibody fragments, diabodies, antibody variants, and antibody-derived binding domains that are part of or associated with other peptides. Antibodies are primarily amino-acid based molecules but can also comprise one or more modifications (including, but not limited to the addition of sugar moieties, fluorescent moieties, chemical tags, etc.). In some cases, antibodies can include non-amino acid-based molecules. Antibodies encompassed by the present invention can be naturally occurring or produced by bioengineering.

In some embodiment, an antibody can comprise a heavy and light variable domain as well as an Fc region. The term “native antibody” refers to a usually heterotetrameric glycoprotein of about 150,000 daltons that is composed of two identical light (L) chains and two identical heavy (H) chains. Each light chain is linked to a heavy chain by one covalent disulfide bond, while the number of disulfide linkages varies among the heavy chains of different immunoglobulin isotypes (e.g., IgG, IgA, IgE and IgM). Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. The rest of the constant domains of a heavy chain of an antibody's two heavy chains compose of the fragment crystallizable (Fc) region of the antibody. The Fc region in the tail region of an antibody interacts with cell surface receptors called Fc receptors and some proteins of the complement system.

The term “light chain” refers to a component of an antibody from any vertebrate species assigned to one of two clearly distinct types, called kappa and lambda, based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these can be further divided into subclasses (isotypes), e.g., IgG1, IgG2, IgG3, IgG4, IgA, and IgA2.

The term “variable domain” refers to specific antibody domains on both the antibody heavy and light chains that differ extensively in sequence among antibodies and are used in the binding and specificity of each particular antibody for its particular antigen. For example, the term “VH” refers to “heavy chain variable domain” and the term “VL” refers to “light chain variable chain.” Variable domains comprise hypervariable regions. The term “hypervariable region” refers to a region within a variable domain comprising amino acid residues responsible for antigen binding. These regions are hypervariable in sequence and/or form structurally defined loops. The amino acids present within the hypervariable regions determine the structure of the complementarity determining regions (CDRs) that become part of the antigen-binding site of the antibody. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies (see, e.g., Xu et al. (2000) Immunity 13, 37-45; Johnson and Wu (2003) Meth. Mol. Biol. 248:1-25). The term “CDR” refers to a region of an antibody comprising a structure that is complimentary to its target antigen or epitope. Other portions of the variable domain that do not interact with the antigen are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments based on comparisons with other antibodies can also be used (Strohl, W. R. Therapeutic Antibody Engineering. Woodhead Publishing, Philadelphia Pa. 2012. Ch. 3, p47-54). Determining residues that make up CDRs can include the use of numbering schemes including, but not limited to, those taught by Kabat (Wu et al. (1970) JEM 132:211-250; Kabat et al. (1992) in “Sequences of Proteins of Immunological Interest,” 5^{th} Edition, U.S. Department of Health and Human Services; Johnson et al. (2000) Nucl. Acids Res. 28:214-218), Chothia (Chothia and Lesk (1987) J. Mol. Biol. 196:901; Chothia et al. (1989) Nature 342:877; Al-Lazikani et al. (1997) J. Mol. Biol. 273:927-948), Lefranc (Lefranc et al. (1995) Immunome Res. 1:3), Honegger (Honegger and Pluckthun (2001) J. Mol. Biol. 309: 657-670), and MacCallum (MacCallum et al. (1996) J. Mol. Biol. 262:732).

VH and VL domains each have three CDRs. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2 and CDR-H3, in order of occurrence when moving from N- to C-terminus along the variable domain polypeptide. Each of CDRs has favored canonical structures, with the exception of the CDR-H3, which comprises amino acid sequences that can be highly variable in sequence and length between antibodies resulting in a variety of three-dimensional structures in antigen-binding domains (Nikoloudis et al. (2014) Peer J. 2:e456). In some cases, CDR-H3s can be analyzed among a panel of related antibodies to assess antibody diversity. Various methods of determining CDR sequences are known in the art and can be applied to known antibody sequences (Strohl, W. R. Therapeutic Antibody Engineering. Woodhead Publishing, Philadelphia Pa. 2012. Ch. 3, p 47-54).

In some embodiments, antibody fragments and variants can comprise any portion of an intact antibody. The terms “antibody fragments” and “antibody variants” also include any synthetic or genetically engineered proteins/polypeptides that act like an antibody by binding to a specific antigen to form a complex. In some embodiments, antibody fragments and variants comprise antigen binding regions from intact antibodies. Examples of antibody fragments can include, but are not limited to Fab, Fab′, F(ab′)₂, and Fv fragments; Fd, diabodies; intrabodies, linear antibodies; single-chain antibody molecules such as single chain variable fragment (scFv); multi-specific antibodies formed from antibody fragments, and the like. Regardless of structure, an antibody fragment or variant binds with the same antigen that is recognized by the parent full-length antibody.

Antibody fragments produced by limited proteolysis of wild-type antibodies are called proteolytic antibody fragments. These include, but are not limited to, Fab fragments, Fab′ fragments and F(ab′)₂ fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin or ficin treatment yields a F(ab′)₂ fragment that has two antigen-binding sites and is still capable of cross-linking antigen. In general, an F(ab′)₂ fragment comprises two “arms,” each of which comprises a variable region that is directed to and specifically binds a common antigen. The two Fab′ molecules are joined by interchain disulfide bonds in the hinge regions of the heavy chains; the Fab′ molecules can be directed toward the same (bivalent) or different (bispecific) epitopes. As used herein, the “Fab′ fragments” contain a single anti-binding domain including an Fab and an additional portion of the heavy chain through the hinge region. Compounds and/or compositions encompassed by the present invention can comprise one or more of these fragments.

The term “Fv” refers to antibody fragments comprising complete antigen-recognition and antigen-binding sites. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain (to form a single chain Fv (scFv) or through the introduction of a disulfide bridge between heavy and light chain variable domains (Strohl, W. R. Therapeutic Antibody Engineering. Woodhead Publishing, Philadelphia Pa. 2012. Ch. 3, p 46-47).

The term “single-chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding. In some embodiments, the VH and VL domains can be linked by a peptide of 10 to 30 amino acid residues. In some embodiments, scFvs are utilized in conjunction with phage display, yeast display or other display methods where they can be expressed in association with a surface member (e.g., phage coat protein) and used in the identification of high affinity peptides for a given antigen. In some embodiments, the term “single-chain antibody” can further include, but is not limited to, a disulfide-linked Fv (dsFv) in which two single-chain antibodies (each of which can be directed to a different epitope) linked together by a disulfide bond. Using molecular genetics, two scFvs can be engineered in tandem into a single polypeptide, separated by a linker domain, called a “tandem scFv” (tascFv). Construction of a tascFv with genes for two different scFvs yields a “bispecific single-chain variable fragments” (bis-scFvs) (Nelson (2010) Mabs 2:77-83). Maxibodies (bivalent scFv fused to the amino terminus of the Fc (CH₂—CH₃ domains) of IgG can also be included.

In some embodiments, the antibody can comprise a modified Fc region. As a non-limiting example, the modified Fc region can be made by the methods or can be any of the regions described in U.S. Pat. Publ. No. US 2015-0065690.

The term “polyclonal antibodies” includes antibodies generated in an immunogenic response to a protein having many epitopes. A composition (e.g., serum) of polyclonal antibodies thus includes a variety of different antibodies directed to the same and to different epitopes within the protein. Methods for producing polyclonal antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-37 to 11-41).

By contrast, the term “monoclonal antibody” refers to an antibody obtained from a population of substantially homogeneous cells (or clones), i.e., the individual antibodies comprising the population are identical and/or bind the same specific epitope of an antigen, except for possible variants that can arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

The term “antibody variant” refers to a modified antibody (in relation to a native or starting antibody) or a biomolecule resembling a native or starting antibody in structure and/or function which includes some differences in their amino acid sequence, composition or structure as compared to the native or starting antibody (e.g., an antibody mimetic). Antibody variants can be altered in their amino acid sequence, composition or structure as compared to a native antibody. Antibody variants can include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments.

In some embodiments, antibodies encompassed by the present invention can comprise antibody fusion proteins. As used herein, the term “antibody fusion protein” is a recombinantly produced antigen-binding molecule in which two or more of the same or different natural antibody, single-chain antibody or antibody fragment segments with the same or different specificities are linked. Valency of the fusion protein indicates the total number of binding arms or sites the fusion protein has to an antigen or epitope; i.e., monovalent, bivalent, trivalent or multivalent. The multivalency of the antibody fusion protein means that it can take advantage of multiple interactions in binding to an antigen, thus increasing the avidity of binding to the antigen. Specificity indicates how many different antigens or epitopes an antibody fusion protein is able to bind, i.e., monospecific, bispecific, trispecific, multispecific, etc. Using these definitions, a natural antibody, e.g., an IgG, is bivalent because it has two binding arms but is monospecific because it binds to one antigen. Monospecific, multivalent fusion proteins have more than one binding site for an epitope but only bind with the same epitope on the same antigen, for example a diabody with two binding sites reactive with the same antigen. The fusion protein can include a multivalent or multispecific combination of different antibody components or multiple copies of the same antibody component. The fusion protein can additionally include a therapeutic agent. Examples of therapeutic agents suitable for such fusion proteins include immunomodulators (“antibody-immunomodulator fusion protein”) and toxins (“antibody-toxin fusion protein”). One preferred toxin comprises a ribonuclease (RNase), preferably a recombinant RNase.

In some embodiments, antibodies encompassed by the present invention can include multispecific antibodies. As used herein, the term “multispecific antibody” refers to an antibody that binds more than one epitope. As used herein, the terms “multibody” or “multispecific antibody” refer to an antibody wherein two or more variable regions bind to different epitopes. The epitopes can be on the same or different targets. In one embodiment, the multispecific antibody can be generated and optimized by the methods described in PCT Publ. No. WO 2011/109726 and U.S. Pat. Publ. No. 2015-0252119. These antibodies are able to bind to multiple antigens with high specificity and high affinity. In some embodiments, a multispecific antibody is a “bispecific antibody.” As used herein, the term “bispecific antibody” refers to an antibody capable of binding two different epitopes on the same or different antigens. In one aspect, bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen. Bispecific antibodies can include any of those described in Riethmuller (2012) Cancer Immun. 12:12-18, Marvin et al. (2005) Acta Pharmacol. Sinica 26:649-658, and Schaefer et al. (2011) Proc. Natl. Acad. Sci. U.S.A. 108:11187-11192. New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

In some embodiments, compositions encompassed by the present invention can include anti-peptide antibodies. As used herein, the term “anti-peptide antibodies” refers to “monospecific antibodies” that are generated in a humoral response to a short (typically, 5 to 20 amino acids) immunogenic polypeptide that corresponds to a few (preferably one) isolated epitopes of the protein from which it is derived (e.g., a target protein encompassed by the present invention). A plurality of antipeptide antibodies includes a variety of different antibodies directed to a specific portion of the protein, i.e., to an amino acid sequence that contains at least one, preferably only one, epitope. Methods for producing antipeptide antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-42 to 11-46).

In some embodiments, antibodies encompassed by the present invention can include diabodies. As used herein, the term “diabody” refers to a small antibody fragment with two antigen-binding sites. Diabodies comprise a heavy chain variable domain VH connected to a light chain variable domain VL in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. U.S.A. 90:6444-6448.

In some embodiments, antibodies encompassed by the present invention can include intrabodies. As used herein, the term “intrabody” refers to a form of antibody that is not secreted from a cell in which it is produced, but instead targets one or more intracellular proteins. Intrabodies can be used to affect a multitude of cellular processes including, but not limited to intracellular trafficking, transcription, translation, metabolic processes, proliferative signaling and cell division. In some embodiments, methods encompassed by the present invention can include intrabody-based therapies. In some such embodiments, variable domain sequences and/or CDR sequences disclosed herein can be incorporated into one or more constructs for intrabody-based therapy. For example, intrabodies can target one or more glycated intracellular proteins or can modulate the interaction between one or more glycated intracellular proteins and an alternative protein. The intracellular expression of intrabodies in different compartments of mammalian cells allows blocking or modulation of the function of endogenous molecules (Biocca et al. (1990) EMBO J. 9:101-108; Colby et al. (2004) Proc. Natl. Acad. Sci. U.S.A. 101: 17616-17621). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. With high specificity and affinity to target antigens, intrabodies have advantages to block certain binding interactions of a particular target molecule, while sparing others. Sequences from donor antibodies can be used to develop intrabodies. Intrabodies are often recombinantly expressed as single domain fragments such as isolated VH and VL domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain intrabodies are often expressed from a recombinant nucleic acid molecule and engineered to be retained intracellularly (e.g., retained in the cytoplasm, endoplasmic reticulum, or periplasm). Intrabodies can be produced using methods known in the art, such as those disclosed and reviewed in, for example, Marasco et al. (1993) Proc. Natl. Acad. Sci. U.S.A. 90:7889-7893; Chen et al. (1994) Hum. Gene Ther. 5:595-601; Chen et al. (1994) Proc. Natl. Acad. Sci. U.S.A. 91:5932-5936; Maciejewski et al. (1995) Nat. Med. 1:667-673; Marasco (1995) Immunotech. 1: 1-19; Mhashilkar et al. (1995) AfBO J. 14: 542-1451; Chen et al. (1996) Hum. Gene Therap. 7:1515-1525; Marasco (1997) Gene 7her. 4:11-15; Rondon and Marasco (1997) Annu. Rev. Microbiol. 51:257-283; Cohen et al. (1998) Oncogene 17:2445-2456; Proba et al. (1998) J Mol. Biol. 275:245-253; Cohen et al. (1998) Oncogene 17:2445-2456; Hassanzadeh et al. (1998) FEBS Lett. 437:81-86; Richardson et al. (1998) Gene Ther. 5:635-644; Ohage and Steipe (1999) J. Mol. Biol. 291:1119-1128; Ohage et al. (1999) J. Mol. Biol. 291:1129-1134; Wirtz and Steipe (1999) Protein Sci. 8:2245-2250; Zhu et al. (1999) J. Immunol. Methods 231:207-222; Arafat et al. (2000) Cancer Gene Ther. 7:1250-1256; der Maur et al. (2002) J. Biol. Chem. 277:45075-45085; Mhashilkar et al. (2002) Gene Ther. 9:307-319; and Wheeler et al. (2003) FASEB J. 17:1733-1735).

In some embodiments, antibodies encompassed by the present invention can include chimeric antibodies. As used herein, the term “chimeric antibody” refers to a recombinant antibody in which a portion of the heavy and light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; Morrison et al. (1984) Proc. Natl. Acad. Sci. U.S.A. 81:6851-6855). For example, a chimeric antibodies of interest herein can include “primatized” antibodies comprising variable domain antigen-binding sequences derived from a non-human primate (e.g., Old World Monkey, such as baboon, rhesus or cynomolgus monkey) and human constant region sequences.

In some embodiments, antibodies encompassed by the present invention can be humanized antibodies. As used herein, the term “humanized antibody” refers to a chimeric antibody comprising a minimal portion from one or more non-human (e.g., murine) antibody source with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity. In one embodiment, the antibody can be a humanized full-length antibody. Humanized antibodies can be generated using protein engineering techniques (e.g., Gussow and Seemann (1991) Meth. Enzymol. 203:99-121). As a non-limiting example, the antibody can have been humanized using the methods taught in U.S. Pat. Publ. No. 2013/0303399.

In some embodiments, antibodies encompassed by the present invention can include cysteine-modified antibodies. In “cysteine-modified antibodies,” a cysteine amino acid is inserted or substituted on the surface of antibody by genetic manipulation and used to conjugate the antibody to another molecule via, e.g., a disulfide bridge. Cysteine substitutions or insertions for antibodies have been described (see, e.g., U.S. Pat. No. 5,219,996). Methods for introducing cysteine residues into the constant region of the IgG antibodies for use in site-specific conjugation of antibodies are described by Stimmel et al. (2000). J. Biol. Chem. 275:330445-30450).

In some embodiments, antibody variants encompassed by the present invention can be antibody mimetics. As used herein, the term “antibody mimetic” refers to any molecule which mimics the function or effect of an antibody and which binds specifically and with high affinity to their molecular targets. In some embodiments, antibody mimetics can be monobodies, designed to incorporate the fibronectin type III domain (Fn3) as a protein scaffold (see U.S. Pat. Nos. 6,673,901 and 6,348,584). In some embodiments, antibody mimetics can include any of those known in the art including, but are not limited to affibody molecules, affilins, affitins, anticalins, avimers, Centyrins, DARPINS™, Fynomers and Kunitz and domain peptides. In other embodiments, antibody mimetics can include one or more non-peptide region.

In some embodiments, antibodies encompassed by the present invention can comprise a single antigen-binding domain. These molecules are extremely small, with molecular weights approximately one-tenth of those observed for full-sized mAbs. Further antibodies can include “nanobodies” derived from the antigen-binding variable heavy chain regions (VHHs) of heavy chain antibodies found in camels and llamas, which lack light chains (see, e.g., Nelson (2010) Mabs 2:77-83).

In some embodiments, antibodies encompassed by the present invention can be “miniaturized.” On example of mAb miniaturization is small modular immunopharmaceuticals (SMIPs). These molecules, which can be monovalent or bivalent, are recombinant single-chain molecules containing one VL, one VH antigen-binding domain, and one or two constant “effector” domains, all connected by linker domains. (see, e.g., Nelson (2010) Mabs 2:77-83). Such a molecule is believed to offer the advantages of increased tissue or tumor penetration claimed by fragments while retaining the immune effector functions conferred by constant domains. Another example of miniaturized antibodies is called a “unibody” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration can minimize the risk of immune activation or oncogenic growth, as IgG4 interacts poorly with FcRs and monovalent unibodies fail to promote intracellular signaling complex formation (see, e.g., Nelson (2010) Mabs 2:77-83).

In some embodiments, antibody variants encompassed by the present invention can be single-domain antibodies (sdAbs, or nanobodies). As used herein the term “sdAb” or “nanobody” refers to an antibody fragment consisting of a single monomeric variable antibody domain. Like a whole antibody, it is able to bind selectively to a specific antigen. In one aspect, a sdAb can be a “Camel Ig or “camelid VHH.” As used herein, the term “camel Ig” refers to the smallest known antigen-binding unit of a heavy chain antibody (Koch-No Ite et al (2007) FASEB J. 21:3490-3498). A “heavy chain antibody” or a “camelid antibody” refers to an antibody that contains two VH domains and no light chains (Hamers-Casterman et al. (1993) Nature 363:446-448 (1993); Sheriff et al. (1996) Nat. Struct. Biol. 3:733-736; Riechmann et al (1999). J. Immunol. Meth. 231:25-38; PCT Publ. Numbers WO1 994/04678 and WO 1994/025591; and U.S. Pat. No. 6,005,079). In another aspect, a sdAb can be a “immunoglobulin new antigen receptor” (IgNAR). The term “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds. Other miniaturized antibody fragments can include “complementary determining region peptides” or “CDR peptides.” A CDR peptide (also known as “minimal recognition unit”) is a peptide corresponding to a single complementarity-determining region (CDR), and can be prepared by constructing genes encoding the CDR of an antibody of interest. Such genes are prepared, for example, by using the polymerase chain reaction to synthesize the variable region from RNA of antibody-producing cells (see, e.g., Larrick et al (1991) Methods Ezymol. 2:106).

Other variants comprising antigen-binding fragments of antibodies can include but are not limited to, disulfide-linked Fvs (sdFv), VL, VH, Camel Ig, V-NAR, VHH, trispecific (Fab₃), bispecific (Fab₂), triabody (trivalent), tetrabody (tetravalent), minibody ((scFv—CH3)₂), bispecific single-chain Fv (Bis-scFv), IgGdeltaCH2, scFv-Fc, (scFv)₂-Fc, affibody, peptide aptamer, avimer or nanobody, or other antigen binding subsequences of an intact immunoglobulin.

In some embodiments, antibodies encompassed by the present invention can be antibodies as described in U.S. Pat. No. 5,091,513. Such an antibody can include one or more sequences of amino acids constituting a region which behaves as a biosynthetic antibody binding site (BABS). The sites comprise 1) non-covalently associated or disulfide bonded synthetic VH and VL dimers, 2) VH-VL or VL-VH single chains wherein the VH and VL are attached by a polypeptide linker, or 3) individuals VH or VL domains. The binding domains comprise linked CDR and FR regions, which can be derived from separate immunoglobulins. The biosynthetic antibodies can also include other polypeptide sequences which function, e.g., as an enzyme, toxin, binding site, or site of attachment to an immobilization media or radioactive atom. Methods are disclosed for producing the biosynthetic antibodies, for designing BABS having any specificity that can be elicited by in vivo generation of antibody, and for producing analogs thereof.

In some embodiments, antibodies encompassed by the present invention can be antibodies with antibody acceptor frameworks taught in U.S. Pat. No. 8,399,625. Such antibody acceptor frameworks can be particularly well suited accepting CDRs from an antibody of interest.

In one embodiment, the antibody can be a conditionally active biologic protein. An antibody can be used to generate a conditionally active biologic protein which are reversibly or irreversibly inactivated at the wild-type normal physiological conditions, as well as to such conditionally active biologic proteins and uses of such conditional active biologic proteins are provided. Such methods and conditionally active proteins are taught in, for example, PCT. Publ. Numbers WO 2015/175375 and WO 2016/036916 and U.S. Pat. Publ. No. 2014/0378660.

The antibodies, as well as variants and/or fragments thereof, as described herein can be produced using recombinant polynucleotides. In one embodiment, the polynucleotides have a modular design to encode at least one of the antibodies, fragments or variants thereof. As a non-limiting example, the polynucleotide construct can encode any of the following designs: (1) the heavy chain of an antibody, (2) the light chain of an antibody, (3) the heavy and light chain of the antibody, (4) the heavy chain and light chain separated by a linker, (5) the VH1, CH1, CH₂, CH₃ domains, a linker and the light chain or (6) the VH1, CHI, CH₂, CH₃ domains, VL region, and the light chain. Any of these designs can also comprise optional linkers between any domain and/or region. The polynucleotides encompassed by the present invention can be engineered to produce any standard class of immunoglobulins using an antibody described herein or any of its component parts as a starting molecule.

In some embodiments, antibodies encompassed by the present invention are therapeutic antibodies. As used herein, the term “therapeutic antibody” means an antibody that is effective in treating a disease or disorder in a mammal with or predisposed to the disease or disorder. An antibody can be a cell penetrating antibody, a neutralizing antibody, an agonist antibody, partial agonist, inverse agonist, partial antagonist or an antagonist antibody.

In some embodiments, antibodies encompassed by the present invention can be naked antibodies. As used herein, the term “naked antibody” is an intact antibody molecule that contains no further modifications such as conjugation with a toxin, or with a chelate for binding to a radionuclide. The Fc portion of the naked antibody can provide effector functions, such as complement fixation and ADCC (antibody dependent cell cytotoxicity), which set mechanisms into action that can result in cell lysis (see, e.g., Markrides (1998) Pharmacol. Rev. 50:59-87).

In some embodiments, antibodies encompassed by the present invention do not have an ADCC activity against cells expressing a biomarker of interest (e.g., biomarkers listed in Table 1 and/or Table 2). In some embodiments, antibodies encompassed by the present invention do not have a CDC activity against cells expressing a biomarker of interest (e.g., biomarkers listed in Table 1 and/or Table 2). In some embodiments, antibodies encompassed by the present invention are not conjugated to another therapeutic moiety (e.g., a cytotoxic agent). In some embodiments, antibodies encompassed by the present invention do not kill cells expressing a biomarker of interest (e.g., biomarkers listed in Table 1 and/or Table 2) upon binding the cells and/or upon internalization by the cells.

b. Antibody Generation

Antibodies encompassed by the present invention can be naturally occurring or man-made through any methods known in the art, such as monoclonal antibodies (mAbs) produced by conventional hybridoma technology, recombinant technology, mutation or optimization of a known antibody, selection from a an antibody library or antibody fragment library, and immunization. The generation of antibodies, whether monoclonal or polyclonal, is well-known in the art. Techniques for the production of antibodies are well-known in the art and described, e.g., in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

Methods of antibody development typically rely on the use of a target molecule for selection, immunization, and/or confirmation of antibody affinity and/or specificity. Target molecules used according to the present invention include target antigens. Target antigens can be amino acid-based molecules, non-amino acid based molecules, or compounds made up of both amino acid-based molecules and non-amino acid-based molecules. The terms “amino acid” and “amino acids” refer to all naturally occurring L-alpha-amino acids as well as non-naturally occurring amino acids. Amino acids are identified by either the one-letter or three-letter designations as follows: aspartic acid (Asp: D), isoleucine (Ile: I), threonine (Thr: T), leucine (Leu: L), serine (Ser: S), tyrosine (Tyr: Y), glutamic acid (Glu: E), phenylalanine (Phe: F), proline (Pro: P), histidine (His: H), glycine (Gly: G), lysine (Lys: K), alanine (Ala: A), arginine (Arg: R), cysteine (Cys: C), tryptophan (Trp: W), valine (Val: V), glutamine (Gin: Q) methionine (Met: M), and asparagine (Asn: N), where the amino acid is listed first followed parenthetically by the three and one letter codes, respectively. Amino acid-based target antigens can be proteins or peptides. As used herein, the term “peptide” refers to an amino-acid based molecule having from 2 to 50 or more amino acids. Special designators apply to the smaller peptides with “dipeptide” referring to a two amino acid molecule and “tripeptide” referring to a three amino acid molecule. Amino acid based molecules having more than 50 contiguous amino acids are considered polypeptides or proteins.

In some embodiments, antibodies can be prepared through immunization of a host with one or more target antigens, which act as immunogens to elicit an immunological response. In some cases, only portions or regions of a given antigen can be used. In the case of amino acid-based antigens, one or more antigen-derived polypeptides or peptides (referred to herein as “antigen peptides”) can be used. Antigen peptides suitable for generating antibodies preferably contain a sequence of at least 4, at least 5, at least 6, at least 7, at least 8, at least 9, at least 10, at least 11, at least 12, at least 13, at least 14, at least 15, from about 5 to about 50 amino acids, from about 10 to about 30 amino acids, from about 10 to about 20 amino acids, from about 40 to about 200 amino acids, or at least 200 amino acids in length. In certain embodiments encompassed by the present invention, where larger polypeptides or proteins are used for generating antibodies, these preferably are at least 50, at least 55, at least 60, at least 70, at least 80, at least 90, or more amino acids in length.

Antibody generation by immunization typically involves the use of non-human animal hosts as subjects for immunization, referred to herein as “immunogenic hosts.” In some embodiments, immunogenic hosts are selected from any vertebrates. In further embodiments, immunogenic hosts are selected from all mammals. In further embodiments, immunogenic hosts are mice, including transgenic or knockout mice. Other immunogenic hosts can include, but are not limited to rats, rabbits, cats, dogs, goats, sheep, hamsters, guinea pigs, cows, horses, pigs, llamas, camels, and chickens.

Immunization of immunogenic hosts with target antigens described herein can comprise the use of one or more adjuvants. Adjuvants can be used to elicit a higher immune response in such immunogenic hosts. As such, adjuvants used according to the present invention can be selected based on their ability to affect antibody titers. Adjuvants can include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and other useful human adjuvants such as BCG (Bacillus Calmette-Guerin) and Corynebacterium parvum. Such adjuvants are also well-known in the art. In some embodiments, water-in-oil emulsions can be useful as adjuvants. Water-in-oil emulsions can act by forming mobile antigen depots, facilitating slow antigen release and enhancing antigen presentation to immune components. Freund's adjuvant can be used as complete Freund's adjuvant (CFA), which comprises mycobacterial particles that have been dried and inactivated, or as incomplete Freund's adjuvant (IFA), lacking such particles. Other water-in-oil-based adjuvants include EMULSIGEN® (MVP Technologies, Omaha, Nebr.). EMULSIGEN® comprises micron-sized oil droplets that are free from animal-based components. It can be used alone or in combination with other adjuvants, including, but not limited to aluminum hydroxide and CARBIGEN™ (MVP Technologies, Omaha, Nebr.). In some embodiments, TITERMAX® adjuvant can be used. TITERMAX® is another water-in-oil emulsion comprising squalene, as well as sorbitan monooleate 80 (as an emulsifier) and other components. In some cases, TITERMAX® can provide higher immune responses, but with decreased toxicity toward immunogenic hosts. Immunostimmulatory oligonucleotides can also be used as adjuvants. Such adjuvants can include, for example, CpG oligodeoxynucleotide (ODN) (Chu et al. (2000) Infect. Immunity 68:1450-1456; ODNs can include any of those available commercially, such as ODN-1585, ODN-1668, ODN-1826, ODN-2006, ODN-2007, ODN-2216, ODN-2336, ODN-2395 and/or ODN-M362, each of which can be purchased, for example, from InvivoGen, San Diego, Calif.) or immune stimulating complexes (ISCOMs), which are spherical open cage-like structures (typically nm in diameter) that are spontaneously formed when mixing together cholesterol, phospholipids and Quillaia saponins under a specific stoichiometry (see, for example, AbISCO-100, Isconova, Uppsala, Sweden). According to embodiments encompassed by the present invention, adjuvant components of immunization solutions can be varied in order to achieve desired results. Such results can include modulating the overall level of immune response and/or level of toxicities in immunogenic hosts.

Monoclonal antibodies encompassed by the present invention can be prepared using well-established methods known by those skilled in the art. In one embodiment, the monoclonal antibodies are prepared using hybridoma technology (Kohler et al. (1975) Nature 256:495-497). In a hybridoma method, a mouse, hamster, or other appropriate immunogenic host animal, is typically immunized with an immunizing agent (e.g., a target antigen) to elicit lymphocytes that produce or are capable of producing antibodies that will specifically bind to the immunizing agent. Alternatively, the lymphocytes can be immunized in vitro. The lymphocytes are then fused with an immortalized cell line using a suitable fusing agent, such as polyethylene glycol, to form a hybridoma cell (Goding, J. W., Monoclonal Antibodies: Principles and Practice. Academic Press. 1986; 59-1031). Immortalized cell lines are usually transformed mammalian cells, particularly myeloma cells of rodent, rabbit, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In some embodiment, immortalized cell lines are those that fuse efficiently, support stable high level expression of antibody by the selected antibody-producing cells, and are sensitive to a medium such as HAT medium. Such cell lines can be murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor et al. (1984) J. Immunol. 133:3001-3005; Brodeur, B. et al., Monoclonal Antibody Production Techniques and Applications. Marcel Dekker, Inc., New York. 1987; 33:51-63). The culture medium in which the hybridoma cells are cultured can then be assayed for the presence of monoclonal antibodies. Preferably, the binding specificity (i.e., specific immunoreactivity) of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known by those skilled in the art. The binding specificity of the monoclonal antibody can, for example, be determined by Scatchard analysis (Munson (1980) Anal. Biochem. 107:220-239). After the desired hybridoma cells are identified, the clones can be subcloned by limiting dilution procedures and grown by standard methods. Suitable culture media for this purpose include, for example, Dulbecco's Modified Eagle's Medium or RPMI-1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, monoclonal antibodies encompassed by the present invention can also be made by recombinant DNA methods, such as those described in U.S. Pat. No. 4,816,567. DNA encoding the monoclonal antibodies encompassed by the present invention can be readily isolated and sequenced using conventional procedures (e.g., by using oligonucleotide probes that are capable of binding specifically to genes encoding the heavy and light chains of murine antibodies). The hybridoma cells encompassed by the present invention serve as a preferred source of DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see, e.g., U.S. Pat. No. 4,816,567) or by covalently joining the immunoglobulin coding sequence with all or part of the coding sequence for a non-immunoglobulin polypeptide. Such a non-immunoglobulin polypeptide can be substituted for the constant domains of an antibody encompassed by the present invention, or can be substituted for the variable domains of an antibody encompassed by the present invention to create a chimeric bivalent antibody.

Antibodies encompassed by the present invention can also be produced by various procedures well-known in the art for the production of polyclonal antibodies. Polyclonal antibody production typically involves immunization of immunogenic host animals, such as rabbits, rats, mice, sheep, or goats, with either free or carrier-coupled immunogens (e.g., target antigens), for example, by intraperitoneal and/or intradermal injection. Injection material is typically an emulsion containing about 100 μg of immunogen or carrier protein. Several booster injections can be needed, for instance, at intervals of about two weeks, to provide a useful titer of antibody which can be detected, for example, by ELISA assay using free peptide adsorbed to a solid surface. The titer of antibodies in serum from an immunized animal can be increased by selection of antibodies, e.g., by adsorption of the peptide onto a solid support and elution of the selected antibodies according to methods well-known in the art.

c. Antibody Selection

A desired antibody can be selected from a larger pool of two or more candidate antibodies based on affinity and/or specificity for target antigens and/or epitopes thereof. In some embodiments, antibody selection can be carried out using an antibody binding assay. Such assays can include, but are not limited to surface plasmon resonance (SPR)-based assays, ELISAs, and flow cytometry-based assays. Assays can utilize a target antigen to bind a desired antibody and then use one or more detection methods to detect binding.

In some embodiments, antibodies encompassed by the present invention can be selected and produced using high throughput methods of discovery. In one embodiment, antibodies encompassed by the present invention are produced through the use of display libraries. The term “display” refers to the expression or “display” of proteins or peptides on the surface of a given display host. The term “library” refers to a collection of unique cDNA sequences. A library can contain from as little as two unique cDNAs to hundreds of billions of unique cDNAs. In some embodiments, detection agents comprising synthetic antibodies are produced using antibody display libraries or antibody fragment display libraries. The term “antibody fragment display library” refers to a display library wherein each member encodes an antibody fragment containing at least one variable region of an antibody. Such antibody fragments are preferably Fab fragments, but other antibody fragments such as single-chain variable fragments (scFvs) are contemplated as well. In a Fab antibody fragment library, each Fab encoded can be identical except for the amino acid sequence contained within the variable loops of the complementarity determining regions (CDRs) of the Fab fragment. In an alternative or additional embodiment, amino acid sequences within the individual VH and/or VL regions can differ as well.

Display libraries can be expressed in a number of possible hosts (referred to herein as “display hosts”) including, but not limited to yeast, bacteriophage (also referred to herein as “phages” or “phage particles,” bacteria and retroviruses. Additional display technologies that can be used include ribosome-display, microbead-display and protein-DNA linkage techniques. When expressed, the Fabs decorate the surface of the host (e.g., phage or yeast) where they can interact with a given target antigen. Any target antigens can be used to select display hosts expressing antibody fragments with the highest affinity for that target. The DNA sequence encoding the variable domains of the bound antibody fragment can then be determined through sequencing using the bound particle or cell. In some embodiments, positive selection is used in the development of antibodies. The term “positive selection” refers to processes by which antibodies and/or fragments thereof are selected from display libraries based on affinity for target antigens containing desirable target sites. In some embodiments, negative selection is utilized in the development of antibodies. The term “negative selection” refers to processes by which non-target agents are used to exclude antibodies and/or fragments thereof from a given display library during antibody development. In some embodiments, both positive and negative selection processes are utilized during multiple rounds of selection in the development of antibodies using display libraries.

In yeast display, cDNA encoding different antibody fragments are introduced into yeast cells where they are expressed and the antibody fragments are “displayed” on the cell surface as described by Chao et al. (2006) Nat. Protoc. 1:755-768. In yeast surface display, expressed antibody fragments contain an additional domain comprising the yeast agglutinin protein, Aga2p. This domain allows the antibody fragment fusion protein to attach to the outer surface of the yeast cell through the formation of disulfide bonds with surface-expressed Aga1p. The result is a yeast cell, coated in a particular antibody fragment. Display libraries of cDNA encoding these antibody fragments are utilized initially in which the antibody fragments each have a unique sequence. These fusion proteins are expressed on the cell surface of millions of yeast cells where they can interact with a desired targets, incubated with the cells. Target peptides can be covalently or otherwise modified with a chemical or magnetic group to allow for efficient cell sorting after successful binding with a suitable antibody fragment takes place. Recovery can be by way of magnetic-activated cell sorting (MACS), fluorescence-activated cell sorting (FACS) or other cell sorting methods known in the art. Once a subpopulation of yeast cells is selected, the corresponding plasmids can be analyzed to determine the sequence of displayed antibody fragments.

Bacteriophage display methods typically utilize filamentous phage including fd, F1 and M13 virions. Such strains are non-lytic, allowing for continued propagation of the host and increased viral titers. Examples of phage display methods that can be used to make the antibodies encompassed by the present invention include those disclosed in Miersch et al. (2012) Methods. 57:486-498; Bradbury et al. (2011) Nat. Biotechnol. 29:245-254; Brinkman et al. (1995) J. Immunol. Meth. 182:41-50; Ames et al. (1995) J. Immunol. Meth. 184:177-186; Kettleborough et al. (1994) Eur. J. Immunol. 24:952-958); Persic et al. (1997) Gene 187:9-18); PCT Publ. Numbers PCT/GB91/01134, WO 90/02809, WO 91/10737, WO 92/01047, WO 92/18619, WO 93/11236, WO 95/15982, and WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

Antibody fragment expression on bacteriophages can be carried out by inserting the cDNA encoding the fragment into the gene expressing a viral coat protein. The viral coat of filamentous bacteriophages is made up of five coat proteins, encoded by a single-stranded genome. Coat protein pIII is the preferred protein for antibody fragment expression, typically at the N-terminus. If antibody fragment expression compromises the function of pIII, viral function can be restored through coexpression of a wild-type pIII, although such expression will reduce the number of antibody fragments expressed on the viral coat, but can enhance access to the antibody fragment by the target. Expression of viral as well as antibody fragment proteins can alternatively be encoded on multiple plasmids. This method can be used to reduce the overall size of infective plasmids and enhance the transformation efficiency. Phage display libraries can comprise millions to billions of phage particles, each expressing unique antibody fragments on their viral coats. Such libraries can provide richly diverse resources that can be used to select potentially hundreds of antibody fragments with diverse levels of affinity for one or more targets (McCafferty et al. (1990) Nature 348:552-554; Edwards et al. (2003). JMB 334:103-118; Schofield et al. (2007) Genome Biol. 8:R254; and Pershad et al. (2010) Prot. Digin. Design Select. 23:279-288). Often, the antibody fragments present in such libraries comprise scFv antibody fragments, comprising a fusion protein of VH and VL antibody domains joined by a flexible linker (e.g., a Ser/Gly-rich linker). These fragments typically comprise the VH domain first, but VL-linker-VH fragments are also contemplated herein. In some cases, scFvs can contain the same sequence with the exception of unique sequences encoding variable loops of the complementarity determining regions (CDRs). In some cases, scFvs are expressed as fusion proteins, linked to viral coat proteins (e.g., the N-terminus of the viral pIII coat protein). VL chains can be expressed separately for assembly with VH chains in the periplasm prior to complex incorporation into viral coats.

In some embodiments, phage enrichment comprises solution-phase phage enrichment where target antigens are present in a solution that is combined with phage solutions. According to such methods, target antigens can comprise detectable labels (e.g., biotin labels) to facilitate retrieval from solution and recovery of bound phage. In other embodiments, solution-phase phage enrichment can comprise the use of targets bound to beads (e.g., streptavidin beads). In some cases, such beads can be magnetic beads to facilitate precipitation. In other embodiments, phage enrichment can comprise solid-phase enrichment where target antigens are immobilized on solid surfaces. According to such methods, phage solutions can be used to contact the solid surface for enrichment with the immobilized targets. Solid surfaces can include any surfaces capable of retaining targets and can include, but are not limited to dishes, plates, flasks, membranes, and tubes. In some cases, immunotubes can be used wherein the inner surface of such tubes are coated with target antigens (e.g., by passing biotinylated targets through streptavidin or neutravidin-coated tubes). Phage enrichment with immunotubes can be carried out by passage of phage solution through the tubes to enrich bound targets.

After selection, bound phage can be used to infect E. coli cultures that are co-infected with helper phage, to produce an amplified output library for the next round of enrichment. This process can be repeated producing narrower and narrower clone sets. In some embodiments, rounds of enrichment are limited to improve the diversity of selected phage. Precipitated library members can be sequenced from the bound phage to obtain cDNA encoding desired scFvs. Such sequences can be directly incorporated into antibody sequences for recombinant antibody production, or mutated and utilized for further optimization through in vitro affinity maturation. IgG antibodies comprising one or more variable domains from selected scFvs can be synthesized for further testing and/or product development. Such antibodies can be produced by insertion of one or more segments of scFv cDNA into expression vectors suited for IgG production.

d. Antibody Engineering

As described above, techniques that can be used to produce antibodies and antibody fragments, such as Fabs and scFvs, are well-known in the art and include those described in U.S. Pat. Nos. 4,946,778 and 5,258,498; Miersch et al. (2012) Methods 57:486-498; Chao et al. (2006) Nat. Protoc. 1:755-768), Huston et al. (1991) Methods Enzymol. 203:46-88; Shu et al. (1993) Proc. Natl. Acad. Sci. U.S.A. 90:7995-7999; and Skerra et al. (1988) Science 240:1038-1041).

After isolation or selection of target antigen-specific antibodies, antibody sequences can be used for recombinant production and/or optimization of such antibodies. In the case of antibody fragment isolation from a display library, coding regions from the isolated fragment can be used to generate whole antibodies, including human antibodies, or any other desired target binding fragment, and expressed in any desired host, including mammalian cells, insect cells, plant cells, yeast, and bacteria, e.g., as described in detail below. If desired, IgG antibodies (e.g., IgG1, IgG2, IgG3 or IgG4) can be synthesized for further testing and/or product development from variable domain fragments produced or selected according to the methods described herein. Such antibodies can be produced by insertion of one or more segments of cDNA encoding desired amino acid sequences into expression vectors suited for IgG production. Expression vectors can comprise mammalian expression vectors suitable for IgG expression in mammalian cells. Mammalian expression of IgGs can be carried out to ensure that antibodies produced comprise modifications (e.g., glycosylation) characteristic of mammalian proteins and/or to ensure that antibody preparations lack endotoxin and/or other contaminants that can be present in protein preparations from bacterial expression systems.

In some embodiments, affinity maturation is performed. The term “affinity maturation” refers to a method whereby antibodies are produced with increasing affinity for a given target through successive rounds of mutation and selection of antibody- or antibody fragment-encoding cDNA sequences. In some cases, this process is carried out in vitro. To accomplish this, amplification of variable domain sequences (in some cases limited to CDR coding sequences) can be carried out using error-prone PCR to produce millions of copies containing mutations including, but not limited to point mutations, regional mutations, insertional mutations and deletional mutations. As used herein, the term “point mutation” refers to a nucleic acid mutation in which one nucleotide within a nucleotide sequence is changed to a different nucleotide. As used herein, the term “regional mutation” refers to a nucleic acid mutation in which two or more consecutive nucleotides are changed to different nucleotides. As used herein, the term “insertional mutation” refers to a nucleic acid mutation in which one or more nucleotides are inserted into a nucleotide sequence. As used herein, the term “deletional mutation” refers to a nucleic acid mutation in which one or more nucleotides are removed from a nucleotide sequence. Insertional or deletional mutations can include the complete replacement of an entire codon or the change of one codon to another by altering one or two nucleotides of the starting codon.

Mutagenesis can be carried out on CDR-encoding cDNA sequences to create millions of mutants with singular mutations in heavy and light chain CDR regions. In another approach, random mutations are introduced only at CDR residues most likely to improve affinity. These newly generated mutagenic libraries can be used to repeat the process to screen for clones that encode antibody fragments with even higher affinity for the target peptide. Continued rounds of mutation and selection promote the synthesis of clones with greater and greater affinity (see, e.g., Chao et al. (2006) Nat. Protoc. 1:755-768).

Affinity matured clones can be selected based on affinity as determined by binding assay (e.g., FACS, ELISA, surface plasmon resonance, etc.). Select clones can then be converted to IgG and tested further for affinity and functional activity. In some cases, the goal of affinity optimization is to increase the affinity by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100 fold, at least 500-fold or at least 1,000-fold or more as compared to the affinity of the original antibody. In cases where optimized affinity is less than desired, the process can be repeated.

In some embodiments, generating chimeric and/or humanized antibodies is useful. For example, for some uses, including the in vivo use of antibodies in humans and in vitro detection assays, it can be preferable to use chimeric, humanized, or human antibodies. A chimeric antibody is a molecule in which different portions of the antibody are derived from different animal species, such as antibodies having a variable region derived from a murine monoclonal immunoglobulin and a human immunoglobulin constant region. Methods for producing chimeric antibodies are well-known in the art (see, e.g., Morrison (1985) Science 229:1202-1207; Gillies et al. (1989)J. Immunol. Meth. 125:191-202.; and U.S. Pat. Nos. 5,807,715; 4,816,567; and 4,816,397).

Humanized antibodies are antibody molecules from non-human species that bind to the desired target and have one or more complementarity determining regions (CDRs) from the nonhuman species and framework regions from a human immunoglobulin molecule. Often, framework residues in the human framework regions are substituted with corresponding residues from the CDR and framework regions of the donor antibody to alter, preferably improve, target binding. These framework substitutions are identified by methods well-known in the art, e.g., by modeling of the interactions of the CDR and framework residues to identify framework residues important for target binding, and by sequence comparison to identify unusual framework residues at particular positions (see, e.g., U.S. Pat. Nos. 5,693,762 and 5,585,089: Riechmann et al. (1988) Nature 332:323-327).

Antibodies can be humanized using a variety of techniques known in the art, including, for example, CDR-grafting (see, e.g., EP Pat. Publ. No. 239,400; PCT Publ. No. WO 91/09967; U.S. Pat. Nos. 5,225,539; 5,530,101; and 5,585,089); veneering or resurfacing (see, e.g., EP Pat. Publ. No. 592,106; EP Pat. Publ. No. 519,596; Padlan (1991) Mol. Immunol. 28:489-498; Studnicka et al. (1994) Protein Eng. 7:805-814; Roguska et al. (1994) Proc. Natl. Acad. Sc. U.S.A. 91:969-973); and chain shuffling (see, e.g., U.S. Pat. No. 5,565,332).

Completely human antibodies are particularly desirable for therapeutic treatment of human patients, so as to avoid or alleviate immune reaction to foreign protein. Human antibodies can be made by a variety of methods known in the art, including the antibody display methods described above, using antibody libraries derived from human immunoglobulin sequences (see, e.g., U.S. Pat. Nos. 4,444,887 and 4,716,111; and PCT Publ. Numbers WO 98/46645, WO 98/50433, WO 98/24893, WO 98/16654, WO 96/34096, WO 96/33735, and WO 91/10741). Human antibodies can also be produced using transgenic mice which are incapable of expressing functional endogenous immunoglobulins, but which can express human immunoglobulin polynucleotides. For example, the human heavy and light chain immunoglobulin polynucleotide complexes can be introduced randomly, or by homologous recombination, into mouse embryonic stem cells. Alternatively, the human variable region, constant region, and diversity region can be introduced into mouse embryonic stem cells, in addition to the human heavy and light chain polynucleotides. The mouse heavy and light chain immunoglobulin polynucleotides can be rendered nonfunctional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected immunogen (e.g., target antigen). Using such a technique, it is possible to produce useful human IgG, IgA, IgM, IgD and IgE antibodies. As illustrated above, methods for producing human antibodies and human monoclonal antibodies and protocols for producing such antibodies are well-known in the art (see also, e.g., PCT Publ. Numbers WO 98/24893, WO 92/01047, WO 96/34096, and WO 96/33735; and U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598).

Once an antibody molecule encompassed by the present invention has been produced by an animal, a cell line, chemically synthesized, or recombinantly expressed, it can be purified (i.e., isolated) by any method known in the art for the purification of an immunoglobulin or polypeptide molecule, for example, by chromatography (e.g., ion exchange, affinity, particularly by affinity for the specific target, Protein A, and sizing column chromatography), centrifugation, differential solubility, or by any other standard technique for the purification of proteins. In addition, the antibodies encompassed by the present invention or fragments thereof can be fused to heterologous polypeptide sequences described herein or otherwise known in the art, to facilitate purification.

In accordance with the present invention, antibodies specifically binding to an antigen can be present in a solution or bound to a substrate. In some embodiments, the antibodies are bound to cellulose nanobeads and confined in one or more detection area of a substrate of a detection device.

e. Antibody Characterization

Antibodies encompassed by the present invention can be characterized by one or more of characteristic selected from the group consisting of structure, isotype, binding (e.g., affinity and specificity), conjugation, glycosylation, and other distinguishing features.

Antibodies encompassed by the present invention can be from any animal origin including birds and mammals. Preferably, such antibodies are of human, murine (e.g., mouse and rat), donkey, sheep, rabbit, goat, guinea pig, camel, horse, or chicken origin. Antibodies encompassed by the present invention can be monospecific or multispecific. Multispecific antibodies can be specific for different epitopes of a peptide encompassed by the present invention, or can be specific for both a peptide encompassed by the present invention, and a heterologous epitope, such as a heterologous peptide or solid support material (see, e.g., PCT Publ. Numbers WO 93/17715, WO 92/08802, WO 91/00360, and WO 92/05793; Tutt et al. (1991). Immunol. 147:60-69; U.S. Pat. Nos. 4,474,893; 4,714,681; 4,925,648; 5,573,920; and 5,601,819; and Kostelny et al. (1992)J. Immunol. 148:1547-1553). For example, the antibodies can be produced against a peptide containing repeated units of a peptide sequence encompassed by the present invention, or they can be produced against a peptide containing two or more peptide sequences encompassed by the present invention, or the combination thereof. As a non-limiting example, a heterobivalent ligand (HBL) system that competitively inhibits antigen binding to mast cell bound IgE antibody, thereby inhibiting mast cell degranulation, has been designed (Handlogten et al. (2011) Chem. Biol. 18:1179-1188).

Antibody characteristics can be determined relative to a standard under normal physiologic conditions, either in vitro or in vivo. Measurements can also be made relative to the presence or absence of the antibodies. Such methods of measuring include standard measurement in tissue or fluids such as serum or blood such as Western blot, enzyme-linked immunosorbent assay (ELISA), activity assays, reporter assays, luciferase assays, polymerase chain reaction (PCR) arrays, gene arrays, real time reverse transcriptase (RT) PCR and the like.

Antibodies can bind or interact with any number of locations on or along a target protein. Antibody target sites contemplated include any and all possible sites on the target protein. Antibodies can be selected for their ability to bind (reversibly or irreversibly) to one or more epitopes on a specific target. Epitopes on targets can include, but are not limited to, one or more feature, region, domain, chemical group, functional group, or moiety. Such epitopes can be made up of one or more atom, group of atoms, atomic structure, molecular structure, cyclic structure, hydrophobic structure, hydrophilic structure, sugar, lipid, amino acid, peptide, glycopeptide, nucleic acid molecule, or any other antigen structure.

f. Antibody Conjugates

In some embodiments, antibodies encompassed by the present invention can be conjugated with one or more detectable label for purposes of detection according to methods well-known in the art. The label can be a radioisotope, fluorescent compound, chemiluminescent compound, enzyme, or enzyme co-factor, or any other labels known in the art. In some embodiments, the antibody that binds to a desired target (also referred to herein as a “primary antibody”) is not labeled, but can be detected by binding of a second antibody that specifically binds to the primary antibody (referred to herein as a “secondary antibody”). According to such methods, the secondary antibody can include a detectable labeled.

In some embodiments, enzymes that can be attached to antibodies can include, but are not limited to horseradish peroxidase (HRP), alkaline phosphatase, and glucose oxidase (GOx). Fluorescent compounds can include, but are not limited to, ethidium bromide; fluorescein and derivatives thereof (e.g., FITC); cyanine and derivatives thereof (e.g., indocarbocyanine, oxacarbocyanine, thiacarbocyanine, and merocyanine); rhodamine; oregon green; eosin; texas red; nile red; nile blue: cresyl violet; oxazine 170; proflavin; acridine orange; acridine yellow; auramine; crystal violet; malachite green; porphin; phthalocyanine; bilirubin; allophycocyanin (APC); green fluorescent protein (GFP) and variants thereof (e.g., yellow fluorescent protein YFP, blue fluorescent protein BFP, and cyan fluorescent protein CFP); ALEXIFLOUR® compounds (Thermo Fisher Scientific, Waltham, Mass.); and quantum dots. Other conjugates that can be used to label antibodies can include biotin, avidin, and streptavidin.

In some embodiments, the present invention encompasses antibody-drug conjugate (ADCs) agents. ADCs are conjugates of an antibody with another moiety such that the agent has targeting ability conferred by the antibody and an additional effect conferred by the moiety. For example, a cytotoxic drug can be tethered to a monoclonal antibody that targets the drug to a cell of interest that contribute to disease progression (e.g., tumor progression) and, upon internalization, releases its toxic payload to the cell. Different effects are achieved based on the conjugated moiety as described above.

4. Small Molecule Agents

In another aspect, small molecule agents are encompassed by the present invention. The small molecule can be an inhibitor, an activator, or a modulator of a biomarker described herein (e.g., one or more targets listed in Table 1 and/or Table 2). The term “small molecule” refers to a low molecular weight (i.e., less than about 900 Daltons) organic compound with a size on the order of 10⁻⁹ m that can help regulate a biological process. In some embodiments, the small molecules can be inhibitors of enzymes (e.g., kinases and transcription factors).

Small molecules can also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. The terms “crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is intended.

Known small molecules that bind to and modulate target genes and/or gene products, as well as information regarding the affected biological activities and pathways can be readily determined from publicly available databases, such as Drugbank, PharmGKB, MedChemExpress, and Selleckchem.

5. Cell-Based Agents

In another aspect, cell-based agents are contemplated. In some embodiments, monocytes and/or macrophages are manipulated, such as being contacted with one or more agents to modulate one or more biomarkers encompassed by the present invention (e.g., one or more targets listed in Table 1 and/or Table 2). For example, cultured cells and/or primary cells can be contacted with agents, processed, and introduced into assays, subjects, and the like. Progeny of such cells are encompassed by the cell-based agents described herein.

In some embodiments, monocytes and/or macrophages are recombinantly engineered to modulate one or more biomarkers encompassed by the present invention (e.g., one or more targets listed in Table 1 and/or Table 2). For example, as describe above, genome editing can be used to modulate the copy number or genetic sequence of a biomarker of interest, such as constitutive or induced knockout or mutation of a biomarker of interest. For example, the CRISPR-Cas system can be used for precise editing of genomic nucleic acids (e.g., for creating non-functional or null mutations). In such embodiments, the CRISPR guide RNA and/or the Cas enzyme can be expressed. For example, a vector containing only the guide RNA can be administered to an animal or cells transgenic for the Cas9 enzyme. Similar strategies can be used (e.g., zinc finger nucleases (ZFNs), transcription activator-like effector nucleases (TALENs), or homing meganucleases (HEs), such as MegaTAL, MegaTev, Tev-mTALEN, CPF1, and the like). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010)Mol. Cell 37:7; U.S. Pat. Publ. Numbers 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

Cell-based agents have an immunocompatibility relationship to a subject host and any such relationship is contemplated for use according to the present invention. For example, the cells, such as adoptive monocytes and/or macrophages, T cells, and the like, can be syngeneic. The term “syngeneic” can refer to the state of deriving from, originating in, or being members of the same species that are genetically identical, particularly with respect to antigens or immunological reactions. These include identical twins having matching MHC types. Thus, a “syngeneic transplant” refers to transfer of cells from a donor to a recipient who is genetically identical to the donor or is sufficiently immunologically compatible as to allow for transplantation without an undesired adverse immunogenic response (e.g., such as one that would work against interpretation of immunological screen results described herein).

A syngeneic transplant can be “autologous” if the transferred cells are obtained from and transplanted to the same subject. An “autologous transplant” refers to the harvesting and reinfusion or transplant of a subject's own cells or organs. Exclusive or supplemental use of autologous cells can eliminate or reduce many adverse effects of administration of the cells back to the host, particular graft versus host reaction.

A syngeneic transplant can be “matched allogeneic” if the transferred cells are obtained from and transplanted to different members of the same species yet have sufficiently matched major histocompatibility complex (MHC) antigens to avoid an adverse immunogenic response. Determining the degree of MHC mismatch can be accomplished according to standard tests known and used in the art. For instance, there are at least six major categories of MHC genes in humans, identified as being important in transplant biology. HLA-A, HLA-B, HLA-C encode the HLA class I proteins while HLA-DR, HLA-DQ, and HLA-DP encode the HLA class I1 proteins. Genes within each of these groups are highly polymorphic, as reflected in the numerous HLA alleles or variants found in the human population, and differences in these groups between individuals is associated with the strength of the immune response against transplanted cells. Standard methods for determining the degree of MHC match examine alleles within HLA-B and HLA-DR, or HLA-A, HLA-B and HLA-DR groups. Thus, tests can be made of at least 4, and even 5 or 6 MHC antigens within the two or three HLA groups, respectively. In serological MHC tests, antibodies directed against each HLA antigen type are reacted with cells from one subject (e.g., donor) to determine the presence or absence of certain MIHC antigens that react with the antibodies. This is compared to the reactivity profile of the other subject (e.g., recipient). Reaction of the antibody with an MHC antigen is typically determined by incubating the antibody with cells, and then adding complement to induce cell lysis (i.e., lymphocytotoxicity testing). The reaction is examined and graded according to the amount of cells lysed in the reaction (see, for example, Mickelson and Petersdorf (1999) Hematopoietic Cell Transplantation, Thomas, E. D. et al. eds., pg 28-37, Blackwell Scientific, Malden, Mass.). Other cell-based assays include flow cytometry using labeled antibodies or enzyme linked immunoassays (ELISA). Molecular methods for determining MHC type are well-known and generally employ synthetic probes and/or primers to detect specific gene sequences that encode the HLA protein. Synthetic oligonucleotides can be used as hybridization probes to detect restriction fragment length polymorphisms associated with particular HLA types (Vaughn (2002) Method. Mol. Biol. MHC Protocol. 210:45-60). Alternatively, primers can be used for amplifying the HLA sequences (e.g., by polymerase chain reaction or ligation chain reaction), the products of which can be further examined by direct DNA sequencing, restriction fragment polymorphism analysis (RFLP), or hybridization with a series of sequence specific oligonucleotide primers (SSOP) (Petersdorf et al. (1998) Blood 92:3515-3520; Morishima et al. (2002) Blood 99:4200-4206; and Middleton and Williams (2002) Method. Mol. Biol. MHC Protocol. 210:67-112).

A syngeneic transplant can be “congenic” if the transferred cells and cells of the subject differ in defined loci, such as a single locus, typically by inbreeding. The term “congenic” refers to deriving from, originating in, or being members of the same species, where the members are genetically identical except for a small genetic region, typically a single genetic locus (i.e., a single gene). A “congenic transplant” refers to transfer of cells or organs from a donor to a recipient, where the recipient is genetically identical to the donor except for a single genetic locus. For example, CD45 exists in several allelic forms and congenic mouse lines exist in which the mouse lines differ with respect to whether the CD45.1 or CD45.2 allelic versions are expressed.

By contrast, “mismatched allogeneic” refers to deriving from, originating in, or being members of the same species having non-identical major histocompatibility complex (MHC) antigens (i.e., proteins) as typically determined by standard assays used in the art, such as serological or molecular analysis of a defined number of MHC antigens, sufficient to elicit adverse immunogenic responses. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members.

Similarly, in contrast, “xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor.

In addition, cells can be obtained from a single source or a plurality of sources (e.g., a single subject or a plurality of subjects). A plurality refers to at least two (e.g., more than one). In still another embodiment, the non-human mammal is a mouse. The animals from which cell types of interest are obtained can be adult, newborn (e.g., less than 48 hours old), immature, or in utero. Cell types of interest can be primary cancer cells, cancer stem cells, established cancer cell lines, immortalized primary cancer cells, and the like. In certain embodiments, the immune systems of host subjects can be engineered or otherwise elected to be immunological compatible with transplanted cancer cells. For example, in one embodiment, the subject can be “humanized” in order to be compatible with human cancer cells. The term “immune-system humanized” refers to an animal, such as a mouse, comprising human HSC lineage cells and human acquired and innate immune cells, survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, neutrophils), DCs, NK cells and mast cells. Representative, non-limiting examples include SCID-hu, Hu-PBL-SCID, Hu-SRC-SCID, NSG (NOD-SCID IL2r-gamma(null) lack an innate immune system, B cells, T cells, and cytokine signaling), NOG (NOD-SCID IL2r-gamma(truncated)), BRG (BALB/c-Rag2(null)IL2r-gamma(null)), and H2dRG (Stock-H2d-Rag2(null)IL2r-gamma(null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98; McCune et al. (1988) Science 241:1632-1639, U.S. Pat. No. 7,960,175, and U.S. Pat. Publ. No. 2006/0161996), as well as related null mutants of immune-related genes like Rag1 (lack B and T cells), Rag2 (lack B and T cells), TCR alpha (lack T cells), perforin (cD8+ T cells lack cytotoxic function), FoxP3 (lack functional CD4+T regulatory cells), IL2rg, or Prf1, as well as mutants or knockouts of PD-1, PD-L1, Tim3, and/or 2B4, allow for efficient engrafiment of human immune cells in and/or provide compartment-specific models of immunocompromised animals like mice (see, for example, PCT Publ. No. WO 2013/062134). In addition, NSG-CD34+(NOD-SCID IL2r-gamma(null) CD34+) humanized mice are useful for studying human gene and tumor activity in animal models like mice.

As used herein, “obtained” from a biological material source means any conventional method of harvesting or partitioning a source of biological material from a donor. For example, biological material can obtained from a solid tumor, a blood sample, such as a peripheral or cord blood sample, or harvested from another body fluid, such as bone marrow or amniotic fluid. Methods for obtaining such samples are well-known to the artisan. In the present invention, the samples can be fresh (i.e., obtained from a donor without freezing). Moreover, the samples can be further manipulated to remove extraneous or unwanted components prior to expansion. The samples can also be obtained from a preserved stock. For example, in the case of cell lines or fluids, such as peripheral or cord blood, the samples can be withdrawn from a cryogenically or otherwise preserved bank of such cell lines or fluid. Such samples can be obtained from any suitable donor.

The obtained populations of cells can be used directly or frozen for use at a later date. A variety of mediums and protocols for cryopreservation are known in the art. Generally, the freezing medium will comprise DMSO from about 5-10%, 10-90/serum albumin, and 50-90% culture medium. Other additives useful for preserving cells include, by way of example and not limitation, disaccharides such as trehalose (Scheinkonig et al. (2004) Bone Marrow Dransplant. 34:531-536), or a plasma volume expander, such as hetastarch (i.e., hydroxyethyl starch). In some embodiments, isotonic buffer solutions, such as phosphate-buffered saline, can be used. An exemplary cryopreservative composition has cell-culture medium with 4% HSA, 7.5% dimethyl sulfoxide (DMSO), and 2% hetastarch. Other compositions and methods for cryopreservation are well-known and described in the art (see, e.g., Broxmeyer et al. (2003) Proc. Natl. Acad. Sci. U.S.A. 100:645-650). Cells are preserved at a final temperature of less than about −135° C.

In some embodiments, the immunotherapy can be CAR (chimeric antigen receptor)-T therapy, where T cells engineered to express CARs comprising an antigen-binding domain specific to an antigen on tumor cells of interest. The term “chimeric antigen receptor” or “CAR” refers to receptors having a desired antigen specificity and signaling domains to propagate intracellular signals upon antigen binding. For example, T lymphocytes recognize specific antigens through interaction of the T cell receptor (TCR) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV, Fav, and another protein binding fragments described herein.

In some embodiments, monocytes and macrophages can be engineered to, for example, express a chimeric antigen receptor (CAR). The modified cell can be recruited to the tumor microenvironment where it acts as a potent immune effector by infiltrating the tumor and killing target cancer cells. The CAR includes an antigen binding domain, a transmembrane domain and an intracellular domain. The antigen binding domain binds to an antigen on a target cell. Examples of cell surface markers that can act as an antigen that binds to the antigen binding domain of the CAR include those associated with viral, bacterial, parasitic infections, autoimmune disease and cancer cells (e.g., tumor antigens).

In one embodiment, the antigen binding domain binds to a tumor antigen, such as an antigen that is specific for a tumor or cancer of interest. Non-limiting examples of tumor associated antigens include BCMA, CD19, CD24, CD33, CD38; CD44v6, CD123, CD22, CD30, CD117, CD171, CEA, CS-1, CLL-1, EGFR, ERBB2, EGFRvill, FLT3, GD2, NY-BR-1, NY-ESO-1, p53, PRSS21, PSMA, ROR1, TAG72, Tn Ag, VEGFR2.

In one embodiment, the transmembrane domain is naturally associated with one or more of the domains in the CAR. The transmembrane domain can be derived either from a natural or from a synthetic source. Transmembrane regions of particular use in this invention can be derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. In some instances, a variety of human hinges can be employed as well including the human Ig (immunoglobulin) hinge.

In one embodiment, the intracellular domain of the CAR includes a domain responsible for signal activation and/or transduction. Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA, CD49a, ITGA4, IA4, CD49D, ITGA6, VLA-6, CD49f, ITGAD, CD 1 id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly08), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

In some embodiments, agents, compositions and methods encompassed by the present invention can be used to re-engineer monocytes and macrophages to increase their ability to present antigens to other immune effector cells, for example, T cells. Engineered monocytes and macrophages as antigen presenting cells (APCs) will process tumor antigens and present antigenic epitopes to T cells to stimulate adaptive immune responses to attack tumor cells.

### V. Uses and Methods

The compositions and agents described herein can be used in a variety of modulatory, therapeutic, screening, diagnostic, prognostic, and therapeutic applications regarding biomarkers described herein (e.g., one or more targets listed in Table 1 and/or Table 2). In any method described herein, such as a modulatory method, therapeutic method, screening method, diagnostic method, prognostic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

In addition, any aspect of the present invention described herein can be performed either alone or in combination with any other aspect of the present invention, including one, more than one, or all embodiments thereof. For example, diagnostic and/or screening methods can be performed alone or in combination with a treatment step, such as providing an appropriate therapy upon determining an appropriate diagnosis and/or screening result.

1. Modulatory and Treatment Methods

One aspect encompassed by the present invention relates to methods of modulating the copy number, amount (e.g., expression), and/or activity (e.g., modulating subcellular localization) of at least one biomarker (e.g., one or more targets listed in Table 1, Table 2, the Examples, etc.) described herein, such as for therapeutic purposes. Such agents can be used to manipulate a particular subpopulation of monocytes and/or macrophages and regulate their numbers and/or activities in a physiological condition, and uses thereof for treating macrophages associated diseases and other clinical conditions. For example, agents, including compositions and pharmaceutical formulations, encompassed by the present invention can modulate the copy number, amount, and/or activity of biomarkers (e.g., at least one target listed in Table 1, Table 2, the Examples, etc.) to thereby modulate the inflammatory phenotype of monocytes and/or macrophages and further modulate immune responses. In some embodiments, cell activities (e.g., cytokine secretion, cell population ratios, etc.) are modulated rather than modulating immune responses per se. Methods for modulating monocyte and macrophage inflammatory phenotypes using the agents, compositions, and formulations disclosed herein, are provided. Accordingly, the agents, compositions and methods can be used for modulating immune responses by modulating the copy number, amount, and/or activity of biomarkers (e.g., at least one target listed in Table 1, Table 2, the Examples, etc.) depletes or enriches for certain types of cells and/or to modulate the ratio of cell types. For example, certain targets listed in Table 1 and/or Table 2 are required for cell survival such that inhibiting the target leads to cell death. Such modulation can be useful for modulating immune responses because the ratio of cell types (e.g., pro-inflammatory versus anti-inflammatory cells) mediating immune responses is modulated. In some embodiments, the agents are used to treat cancer in a subject afflicted with a cancer.

The present disclosure demonstrates that the downregulation of the expression of these genes in macrophages can re-polarize (e.g., change the phenotype of) the macrophages. In some embodiments, the phenotype of an M2 macrophage is changed to result in a macrophage with a Type 1 (M2-like) or M1 phenotype, or vice versa regarding M1 macrophages and Type 2 (M2-like) or M2 phenotypes. In some embodiments, agents encompassed by the present invention are used to modulate (e.g., inhibit) the trafficking, polarization, and/or activation of monocytes and macrophages with an M2 phenotype, or vice versa regarding Type 1 and M1 macrophages. The present invention further provides method for reducing populations of monocytes and/or macrophages of interest, such as M1 macrophages, M2 macrophages (e.g., TAMs in a tumor), and the like.

In some embodiments, the present invention provides methods for changing the distribution of monocytes and/or macrophages, including subtypes thereof, such as pro-tumoral macrophages and anti-tumoral macrophages. In one example, the present invention provides methods for driving macrophages towards a pro-inflammatory immune response from an anti-inflammatory immune response and vice versa. Cell types can be depleted and/or enriched by 5%, 10%, 15%, 20%, 25%, 30%, 35%, 40%, 45%, 50%, 55%, 60%, 65%, 70%, 75%, 80%, 85%, 90%, 95%, 99%, or more, or any range in between inclusive, such as 45-55%.

In some embodiments, the modulation occurs in cells, such as monocyte, macrophage, or other phagocyte, like a dendritic cell. In some embodiments, the cell is a macrophage subtype, such as a macrophage subtype described herein. For example, the macrophage can be a tissue resident macrophage (TAM) or a macrophage derived from a circulating monocyte in the bloodstream.

In some embodiments, modulating monocyte and/or macrophage inflammatory phenotypes results in desired modulated immune responses, such as modulation of abnormal monocyte migration and proliferation, unregulated proliferation of tissue resident macrophages, unregulated pro-inflammatory macrophages, unregulated anti-inflammatory macrophages, unbalanced distribution of pro-inflammatory and anti-inflammatory macrophage subpopulations in a tissue, an abnormally adopted activation state of monocytes and macrophages in a disease condition, modulated cytotoxic T-cell activation and function, overcoming of resistance of cancer cells to therapy, and sensitivity of cancer cells to immunotherapy, such as immune checkpoint therapy. In some embodiments, such phenotypes are reversed.

Methods for treating and/or preventing a disease associated with monocytes and macrophages comprise contacting cells, either in vitro, ex vivo, or in vivo (e.g., administering to a subject), with agents and compositions encompassed by the present invention, wherein the agents and compositions manipulate the migration, recruitment, differentiation and polarization, activation, function, and/or survival of monocytes and macrophages. In some embodiments, modulating one or more biomarkers encompassed by the present invention is used to modulate (e.g., inhibit or deplete) the proliferation, recruitment, polarization, and/or activation of monocytes and macrophages in a tissue microenvironment, such as tumor tissue.

In one aspect encompassed by the present invention, methods for reducing anti-inflammatory activities of monocytes and/or macrophages are provided.

In another aspect encompassed by the present invention, methods for increasing pro-inflammatory activities of monocytes and/or macrophages are provided.

In another aspect encompassed by the present invention, methods for balancing pro-inflammatory monocytes and macrophages and anti-inflammatory monocytes and macrophages in a tissue are provided.

Modulatory methods encompassed by the present invention involve contacting a cell with one or more modulators of a biomarker encompassed by the present invention, including at least one biomarker (e.g., at least one target listed in Table 1 and/or Table 2) encompassed by the present invention, including at least one biomarker (e.g., at least one target listed in Table 1 and/or Table 2) listed in Table 1, Table 2, and the Examples, or a fragment thereof or agent that modulates one or more of the activities of biomarker activity associated with the cell. An agent that modulates biomarker activity can be an agent as described herein, such as a nucleic acid or a polypeptide, a naturally-occurring binding partner of the biomarker, an antibody against the biomarker, a combination of antibodies against the biomarker and antibodies against other immune related targets, at least one biomarker (e.g., at least one target listed in Table 1 and/or Table 2) agonist or antagonist, a peptidomimetic of at least one biomarker (e.g, at least one target listed in Table 1 and/or Table 2) agonist or antagonist, at least one biomarker (e.g., at least one target listed in Table 1 and/or Table 2) peptidomimetic, other small molecule, or small RNA directed against or a mimic of at least one biomarker (e.g., at least one target listed in Table 1 and/or Table 2) nucleic acid gene expression product.

An agent that modulates the expression of at least one biomarker (e.g., at least one target listed in Table 1 and/or Table 2) encompassed by the present invention, including at least one biomarker (e.g., at least one target listed in Table 1 and/or Table 2) encompassed by the present invention, including at least one biomarker (e.g., at least one target listed in Table 1 and/or Table 2) listed in Table 1, Table 2, and the Examples, or a fragment thereof is, e.g., an antisense nucleic acid molecule, RNAi molecule, shRNA, mature miRNA, pre-miRNA, pri-miRNA, miRNA*, anti-miRNA, or a miRNA binding site, or a variant thereof, or other small RNA molecule, triplex oligonucleotide, ribozyme, or recombinant vector for expression of at least one biomarker (e.g., at least one target listed in Table 1 and/or Table 2) polypeptide. For example, an oligonucleotide complementary to the area around at least one biomarker (e.g., at least one target listed in Table 1 and/or Table 2) polypeptide translation initiation site can be synthesized. One or more antisense oligonucleotides can be added to cell media, typically at 200 μg/ml, or administered to a patient to prevent the synthesis of at least one biomarker (e.g., at least one target listed in Table 1 and/or Table 2) polypeptide. The antisense oligonucleotide is taken up by cells and hybridizes to at least one biomarker (e.g, at least one target listed in Table 1 and/or Table 2) mRNA to prevent translation. Alternatively, an oligonucleotide which binds double-stranded DNA to form a triplex construct to prevent DNA unwinding and transcription can be used. As a result of either, synthesis of biomarker polypeptide is blocked. When biomarker expression is modulated, such modulation can occur by a means other than by knocking out the biomarker gene.

Agents that modulate expression, by virtue of the fact that they control the amount of biomarker in a cell, also modulate the total amount of biomarker activity in a cell.

In one embodiment, the agent stimulates one or more activities of at least one biomarker (e.g., at least one target listed in Table 1 and/or Table 2) encompassed by the present invention, including at least one biomarker (e.g., at least one target listed in Table 1 and/or Table 2) listed in Table 1 and the Examples or a fragment thereof. Examples of such stimulatory agents include active biomarker polypeptide or a fragment thereof and a nucleic acid molecule encoding the biomarker or a fragment thereof that has been introduced into the cell (e.g., cDNA, mRNA, shRNAs, siRNAs, small RNAs, mature miRNA, pre-miRNA, pri-miRNA, miRNA*, anti-miRNA, or a miRNA binding site, or a variant thereof, or other functionally equivalent molecule known to a skilled artisan). In another embodiment, the agent inhibits one or more biomarker activities. In one embodiment, the agent inhibits or enhances the interaction of the biomarker with its natural binding partner(s). Examples of such inhibitory agents include antisense nucleic acid molecules, anti-biomarker antibodies, biomarker inhibitors, and compounds identified in the screening assays described herein.

In some embodiments, the one or more biomarkers is one or more, two or more, three or more, four or more, etc. up to and including all of the biomarkers described herein and any range in between, such as 2-4 targets listed in Table 1 and/or Table 2.

These modulatory methods can be performed in vitro (e.g., by contacting the cell with the agent) or, alternatively, by contacting an agent with cells in vivo (e.g., by administering the agent to a subject). In some embodiments, agents, compositions and methods encompassed by the present invention can be used to modulate monocytes and/or macrophages during vaccination. Vaccine protection often requires the induction of pro-inflammatory cytokines. One potential therapeutic intervention can be to manipulate monocyte and/or macrophage populations during vaccination, for example, to minimize the induction of regulatory macrophages.

a. Subjects

The present invention provides methods of treating an individual afflicted with a condition or disorder that would benefit from up- or down-modulation of at least one biomarker (e.g., at least one target listed in Table 1 and/or Table 2) encompassed by the present invention listed in Table 1 and/or Table 2 and the Examples or a fragment thereof, e.g., a disorder characterized by unwanted, insufficient, or aberrant expression or activity of the biomarker or fragments thereof. In one embodiment, the method involves administering an agent (e.g., an agent identified by a screening assay described herein), or combination of agents that modulates (e.g., upregulates or downregulates) biomarker expression or activity. In another embodiment, the method involves administering at least one biomarker (e.g., at least one target listed in Table 1 and/or Table 2) polypeptide or nucleic acid molecule as therapy to compensate for reduced, aberrant, or unwanted biomarker expression or activity. Subjects in need of therapy can be treated according to methods described herein and additional methods, such as those also described herein, can be combined with such therapeutic methods, such as methods to diagnose, prognose, monitor, and the like (e.g., modulation of populations of monocytes and/or macrophages confirmed to have expression of the biomarker of interest, and subjects comprising such monocytes and/or macrophages).

Stimulation of biomarker activity is desirable in situations in which the biomarker is abnormally downregulated and/or in which increased biomarker activity is likely to have a beneficial effect. Likewise, inhibition of biomarker activity is desirable in situations in which biomarker is abnormally upregulated and/or in which decreased biomarker activity is likely to have a beneficial effect.

In some embodiments, the subject is an animal. The animal can be of either sex and can be at any stage of development. In some embodiments, the animals is a vertebrate, such as a mammal. In some embodiments, the subject is a non-human mammal. In some embodiments, the subject is a domesticated animal, such as a dog, cat, cow, pig, horse, sheep, or goat. In some embodiments, the subject is a companion animal, such as a dog or cat. In some embodiments, the subject is a livestock animal, such as a cow, pig, horse, sheep, or goat. In some embodiments, the subject is a zoo animal. In some embodiments, the subject is a research animal, such as a rodent (e.g., mouse or rat), dog, pig, or non-human primate. In some embodiments, the animal is a genetically engineered animal. In some embodiments, the animal is a transgenic animal (e.g., transgenic mice and transgenic pigs). In some embodiments, the subject is a fish or reptile. In some embodiments, the subject is a human. In some embodiments, the subject is an animal model of cancer. For example, the animal model can be an orthotopic xenograft animal model of a human-derived cancer.

In some embodiments of the methods encompassed by the present invention, the subject has not undergone treatment, such as chemotherapy, radiation therapy, targeted therapy, and/or immunotherapies. In some embodiments, the subject has undergone treatment, such as chemotherapy, radiation therapy, targeted therapy, and/or immunotherapies.

In some embodiments, the subject has had surgery to remove cancerous or precancerous tissue. In some embodiments, the cancerous tissue has not been removed, e.g., the cancerous tissue can be located in an inoperable region of the body, such as in a tissue that is essential for life, or in a region where a surgical procedure would cause considerable risk of harm to the patient.

In some embodiments, the subject or cells thereof are resistant to a therapy of relevance, such as resistant to immune checkpoint inhibitor therapy. For example, modulating one or more biomarkers encompassed by the present invention can overcome resistance to immune checkpoint inhibitor therapy.

In some embodiments, the subjects are in need of modulation according to compositions and methods described herein, such as having been identified as having an unwanted absence, presence, or aberrante expression and/or activity of one or more biomarkers described herein.

In some embodiments, the subjects have a solid tumor that is infiltrated with macrophages that represent at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, or more, or any range in between, inclusive, such as at least about 5% to at least about 20%, of the mass, volume, and/or number of cells in the tumor or the tumor microenvironment. Such cells can be any described as being useful in other embodiments herein, such as Type 1 macrophages, M1 macrophages, TAMs, monocytes and/or macrophages expressing CD11b or CD14 or both CD11 and CD14, and the like.

The methods encompassed by the present invention can be used to determine the responsiveness to cancer therapy (e.g., at least one modulator of biomarkers listed in Table 1 and/or Table 2) of many different cancers in subjects such as those described herein.

In addition, these modulatory agents can also be administered in combination therapy to further modulate a desired activity. For examples, agents and compositions that target to IL-4, IL-4Rα, IL-13, and CD40 can be used to modulate monocyte and/or macrophage differentiation and/or polarization. Agents and compositions that target to CD11b, CSF-1R, CCL2, neurophilim-1 and ANG-2 can be used to modulate macrophage recruitment to a tissue. Agents and compositions that target to IL-6, IL-6R and TNF-α can be used to modulate macrophage function. Additional agents include, without limitations, chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

b. Cancer Therapies

In some embodiments, agents encompassed by the present invention are used to treat cancer. For example, the present invention provides methods for reducing pro-tumoral functions of monocytes and/or macrophages (i.e., tumorigenicity) and/or increasing anti-tumoral functions of monocytes and/or macrophages. In some particular embodiments, the method encompassed by the present invention can reduce at least one of the pro-tumoral functions of macrophages including 1) recruitment and polarization of tumor associate macrophages (TAMs), 2) tumor angiogenesis, 3) tumor growth, 4) tumor cell differentiation, 5) tumor cell survival, 6) tumor invasion and metastasis, 7) immune inhibition, and 8) immunosuppressive tumor microenvironment.

Cancer therapy (e.g., at least one modulator of one or more targets listed in Table 1 and/or Table 2) or combinations of therapies (e.g., at least one modulator of one or more targets listed in Table 1 and/or Table 2, in combination with at least one immunotherapy) can be used to contact cancer cells and/or administered to a desired subject, such as a subject that is indicated as being a likely responder to cancer therapy (e.g., at least one modulator of one or more targets listed in Table 1 and/or Table 2). In another embodiment, such cancer therapy (e.g., at least one modulator of one or more targets listed in Table 1 and/or Table 2) can be avoided once a subject is indicated as not being a likely responder to the cancer therapy (e.g., at least one modulator of one or more targets listed in Table 1 and/or Table 2) and an alternative treatment regimen, such as targeted and/or untargeted cancer therapies can be administered. Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with or without cancer therapy (e.g., at least one modulator of one or more targets listed in Table 1 and/or Table 2).

Representative exemplary agents useful for modulating biomarkers encompassed by the present invention (e.g., one or more targets listed in Table 1 and/or Table 2), are described above. As described further below, anti-cancer agents encompass biotherapeutic anti-cancer agents (e.g., interferons, cytokines (e.g., tumor necrosis factor, interferon α, interferon γ, etc.), vaccines, hematopoietic growth factors, monoclonal serotherapy, immunostimulants and/or immunodulatory agents (e.g., IL-1, 2, 4, 6, and/or 12), immune cell growth factors (e.g., GM-CSF), and antibodies (e.g., trastuzumab, T-DM1, bevacizumab, cetuximab, panitumumab, rituximab, tositumomab, and the like), as well as chemotherapeutic agents.

The term “targeted therapy” refers to administration of agents that selectively interact with a chosen biomolecule to thereby treat cancer. For example, targeted therapy regarding the inhibition of immune checkpoint inhibitor is useful in combination with the methods encompassed by the present invention.

The term “immunotherapy” or “immunotherapies” generally refers to any strategy for modulating an immune response in a beneficial manner and encompasses the treatment of a subject afflicted with, or at risk of contracting or suffering a recurrence of, a disease by a method comprising inducing, enhancing, suppressing or otherwise modifying an immune response, as well as any treatment that uses certain parts of a subject's immune system to fight diseases, such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” In some embodiments, an immunotherapy is specific for cells of interest, such as cancer cells. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Some forms of immunotherapy are targeted therapies that can comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They can also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer. Similarly, immunotherapy can take the form of cell-based therapies. For example, adoptive cellular immunotherapy is a type of immunotherapy using immune cells, such as T cells, that have a natural or genetically engineered reactivity to a patient's cancer are generated and then transferred back into the cancer patient. The injection of a large number of activated tumor-specific T cells can induce complete and durable regression of cancers.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, an immunotherapeutic agent is an agonist of an immune-stimulatory molecule; an antagonist of an immune-inhibitory molecule; an antagonist of a chemokine; an agonist of a cytokine that stimulates T cell activation; an agent that antagonizes or inhibits a cytokine that inhibits T cell activation; and/or an agent that binds to a membrane bound protein of the B7 family. In some embodiments, the immunotherapeutic agent is an antagonist of an immune-inhibitory molecule. In some embodiments, the immunotherapeutic agents can be agents for cytokines, chemokines and growth factors, for examples, neutralizing antibodies that neutralize the inhibitory effect of tumor associated cytokines, chemokines, growth factors and other soluble factors including IL-10, TGF-β and VEGF.

In some embodiments, immunotherapy comprises inhibitors of one or more immune checkpoints. The term “immune checkpoint” refers to a group of molecules on the cell surface of CD4+ and/or CD8+ T cells that fine-tune immune responses by modulating anti-cancer immune responses, such as down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD200R, CD160, gp49B, PIR-B, KRLG-1, KIR family receptors, TIM-1, TIM-3, TIM-4, LAG-3 (CD223), IDO, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624).

Some immune checkpoints are “immune-inhibitory immune checkpoints” encompassing molecules (e.g., proteins) that inhibit, down-regulate, or suppress a function of the immune system (e.g., an immune response). For example, PD-L1 (programmed death-ligand 1), also known as CD274 or B7-H1, is a protein that transmits an inhibitory signal that reduces proliferation of T cells to suppress the immune system. CTLA-4 (cytotoxic T-lymphocyte-associated protein 4), also known as CD152, is a protein receptor on the surface of antigen-presenting cells that serves as an immune checkpoint (“off” switch) to downregulate immune responses. TIM-3 (T-cell immunoglobulin and mucin-domain containing-3), also known as HAVCR2, is a cell surface protein that serves as an immune checkpoint to regulate macrophage activation. VISTA (V-domain Ig suppressor of T cell activation) is a type I transmembrane protein that functions as an immune checkpoint to inhibit T cell effector function and maintain peripheral tolerance. LAG-3 (lymphocyte-activation gene 3) is an immune checkpoint receptor that negatively regulates proliferation, activation, and homeostasis of T cells. BTLA (B- and T-lymphocyte attenuator) is a protein that displays T cell inhibition via interactions with tumor necrosis family receptors (TNF-R). KIR (killer-cell immunoglobulin-like receptor) is a family of proteins expressed on NK cells, and a minority of T cells, that suppress the cytotoxic activity of NK cells. In some embodiments, immunotherapeutic agents can be agents specific to immunosuppressive enzymes such as inhibitors that can block the activities of arginase (ARG) and indoleamine 2,3-dioxygenase (IDO), an immune checkpoint protein that suppresses T cells and NK cells, which change the catabolism of the amino acids arginine and tryptophan in the immunosuppressive tumor microenvironment. The inhibitors can include, but are not limited to, N-hydroxy-L-Arg (NOHA) targeting to ARG-expressing M2 macrophages, nitroaspirin or sildenafil (Viagra®), which blocks ARG and nitric oxide synthase (NOS) simultaneously; and IDO inhibitors, such as 1-methyl-tryptophan. The term further encompasses biologically active protein fragment, as well as nucleic acids encoding full-length immune checkpoint proteins and biologically active protein fragments thereof. In some embodiment, the term further encompasses any fragment according to homology descriptions provided herein.

By contrast, other immune checkpoints are “immune-stimulatory” encompassing molecules (e.g., proteins) that activate, stimulate, or promote a function of the immune system (e.g., an immune response). In some embodiments, the immune-stimulatory molecule is CD28, CD80 (B7.1), CD86 (B7.2), 4-1BB (CD137), 4-1BBL (CD137L), CD27, CD70, CD40, CD40L, CD122, CD226, CD30, CD30L, OX40, OX40L, HVEM, BTLA, GITR and its ligand GITRL, LIGHT, LTpR, LTas, ICOS (CD278), ICOSL (B7-H2), and NKG2D. CD40 (cluster of differentiation 40) is a costimulatory protein found on antigen presenting cells that is required for their activation. OX40, also known as tumor necrosis factor receptor superfamily member 4 (TNFRSF4) or CD134, is involved in maintenance of an immune response after activation by preventing T-cell death and subsequently increasing cytokine production. CD137 is a mgember of the tumor necrosis factor receptor (TNF-R) family that co-stimulates activated T cells to enhance proliferation and T cell survival. CD122 is a subunit of the interleukin-2 receptor (IL-2) protein, which promotes differentiation of immature T cells into regulatory, effector, or memory T cells. CD27 is a member of the tumor necrosis factor receptor superfamily and serves as a co-stimulatory immune checkpoint molecule. CD28 (cluster of differentiation 28) is a protein expressed on T cells that provides co-stimulatory signals required for T cell activation and survival. GITR (glucocorticoid-induced TNFR-related protein), also known as TNFRSF18 and AITR, is a protein that plays a key role in dominant immunological self-tolerance maintained by regulatory T cells. ICOS (inducible T-cell co-stimulator), also known as CD278, is a CD28-superfamily costimulatory molecule that is expressed on activated T cells and play a role in T cell signaling and immune responses.

Immune checkpoints and their sequences are well-known in the art and representative embodiments are described further below. Immune checkpoints generally relate to pairs of inhibitory receptors and the natural binding partners (e.g., ligands). For example, PD-1 polypeptides are inhibitory receptors capable of transmitting an inhibitory signal to an immune cell to thereby inhibit immune cell effector function, or are capable of promoting costimulation (e.g, by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells. The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell inhibitory signals, and the ability to modulate the immune response. The term “PD-1 ligand” refers to binding partners of the PD-1 receptor and includes both PD-L1 (Freeman et al. (2000). J. Exp. Med. 192:1027-1034) and PD-L2 (Latchman et al. (2001) Nat. Immunol. 2:261). The term “PD-1 ligand activity” includes the ability of a PD-1 ligand polypeptide to bind its natural receptor(s) (e.g., PD-1 or B7-1), the ability to modulate immune cell inhibitory signals, and the ability to modulate the immune response.

As used herein, the term “immune checkpoint therapy” refers to the use of agents that inhibit immune-inhibitory immune checkpoints, such as inhibiting their nucleic acids and/or proteins. Inhibition of one or more such immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins that block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g., the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. Therapeutic agents used for blocking the PD-1 pathway include antagonistic antibodies and soluble PD-L1 ligands. The antagonist agents against PD-1 and PD-L1/2 inhibitory pathway can include, but are not limited to, antagonistic antibodies to PD-1 or PD-L1/2 (e.g., 17D8, 2D3, 4H1, 5C4 (also known as nivolumab or BMS-936558), 4A11, 7D3 and 5F4 disclosed in U.S. Pat. No. 8,008,449; AMP-224, pidilizumab (CT-011), pembrolizumab, and antibodies disclosed in U.S. Pat. Nos. 8,779,105; 8,552,154; 8,217,149; 8,168,757; 8,008,449; 7,488,802; 7,943,743; 7,635,757; and 6,808,710. Similarly, additional representative checkpoint inhibitors can be, but are not limited to, antibodies against inhibitory regulator CTLA-4 (anti-cytotoxic T-lymphocyte antigen 4 anti-cytotoxic T-lymphocyte antigen 4), such as ipilimumab, tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and other antibodies, such as those disclosed in U.S. Pat. Nos. 8,748,815; 8,529,902; 8,318,916; 8,017,114; 7,744,875; 7,605,238; 7,465,446, 7,109,003; 7,132,281; 6,984,720; 6,682,736; 6,207,156; and 5,977,318, as well as EP Pat. No. 1212422, U.S. Pat Publ. Numbers 2002/0039581 and 2002/086014, and Hurwitz et al. (1998) Proc. Natl. Acad. Sci. U.S.A. 95:10067-10071.

The representative definitions of immune checkpoint activity, ligand, blockade, and the like exemplified for PD-1, PD-L1, PD-L2, and CTLA-4 apply generally to other immune checkpoints.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent can be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary agents include, but are not limited to, alkylating agents: nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Similarly, additional exemplary agents including platinum-ontaining compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AGO13736), bosutinib (SKI-606), cediranib (RECENTIN®, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC1-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. Compositions comprising one or more chemotherapeutic agents (e.g., FLAG, CHOP) can also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.): PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard et. al. (2003) Exp. Hematol. 31:446-454); Herceg (2001) Mut. Res. 477:97-110). Poly(ADP-ribose) polymerase 1 (PARP1) is a key molecule in the repair of DNA single-strand breaks (SSBs) (de Murcia J. et al. (1997) Proc. Natl. Acad Sci. U.S.A. 94:7303-7307; Schreiber et al. (2006) Nat. Rev. Mol. Cell Biol. 7:517-528; Wang et al. (1997) Genes Dev. 11:2347-2358). Knockout of SSB repair by inhibition of PARP1 function induces DNA double-strand breaks (DSBs) that can trigger synthetic lethality in cancer cells with defective homology-directed DSB repair (Bryant et al. (2005) Nature 434:913-917; Farmer et al. (2005) Nature 434:917-921). The foregoing examples of chemotherapeutic agents are illustrative and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat can help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area can be heated externally with high-frequency waves aimed at a tumor from a device outside the body. To achieve internal heating, one of several types of sterile probes can be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It can also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and can be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time can be needed because the precision of the laser allows for a smaller incision. Healing time is often shortened; since laser heat seals blood vessels, there is less bleeding, swelling, or scarring. Laser surgery can be less complicated. For example, with fiber optics, laser light can be directed to parts of the body without making a large incision. More procedures can be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They can be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also can be used with low-power microscopes, giving the doctor a clear view of the site being treated. Used with other instruments, laser systems can produce a cutting area as small as 200 microns in diameter-less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers can be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat can help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with cancer therapy (e.g., at least one modulator of biomarkers listed in Table 1 and/or Table 2) can vary according to the particular modulator of biomarkers listed in Table 1 and/or Table 2 or combination thereof. An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods encompassed by the present invention is a factor in determining optimal treatment doses and schedules.

2. Screening Methods

Another aspect encompassed by the present invention encompasses screening assays.

In some embodiments, methods are provided for selecting agents (e.g., antibodies, fusion proteins, peptides, or small molecules) which modulate the copy number, amount, and/or activity of one or more biomarkers encompassed by the present invention (e.g., one or more targets listed in Table 1 and/or Table 2) in monocytes and/or macrophages. In some embodiments, the selected agents also modulate immune responses mediated by such monocytes and/or macrophages (e.g., modulating CD8+ cyototoxic T cell killing; modulating sensitivity of cancer cells to immune checkpoint therapy; modulating resistance to anti-cancer therapies like immunecheckpoint therapy; modulating the modulating cancer therapy; modulating immune cell micgration, recruitment, differentiation, and/or survival, such as of NK, neutrophil, and macrophage cells; and the like). Thus, any diagnostic, prognostic, or screening method described herein can use biomarkers described herein as readouts of a desired phenotype, such as modulated immune phenotype, as well as agents that modulate the copy number, amount, and/or activity of one or more biomarkers described herein to confirm modulation of the one or more biomarkers and/or to confirm the effects of the agents on readouts of a desired phenotype, such as modulated immune responses, sensitivity to immune checkpoint blockade, and the like. Such methods can utilize screening assays, including cell-based and non-cell based assays.

For example, a method for screening for agents that sensitize cancer cells to cytotoxic T cell-mediated killing and/or immune checkpoint therapy comprising a) contacting cancer cells with cytotoxic T cells and/or immune checkpoint therapy in the presence of monocytes and/or macrophages contacted with i) at least one agent that decreases the copy number, amount, and/or activity of at least one target listed in Table 1 and/or ii) at least one agent that increases the copy number, amount, and/or activity of the at least one target listed in Table 2; b) contacting cancer cells with cytotoxic T cells and/or immune checkpoint therapy in the presence of control monocytes and/or macrophages that are not contacted with the at least one agent or agents; and c) identifying agents that sensitize cancer cells to cytotoxic T cell-mediated killing and/or immune checkpoint therapy by identifying agents that increase cytotoxic T cell-mediated killing and/or immune checkpoint therapy efficacy (such as cell killing) in a) compared to b), is provided.

In some embodiments, the assays are directed to identifying agents that inhibit immune cell proliferation and/or effector function, or to induce anergy, clonal deletion, and/or exhaustion by assaying the opposite modulation effect of the one or more biomarkers. The present invention further encompasses methods of inhibiting immune cell proliferation and/or effector function, or to induce anergy, clonal deletion, and/or exhaustion through such a modulation.

In another example, a method for screening for agents that sensitize cancer cells to cytotoxic T cell-mediated killing and/or immune checkpoint therapy comprising a) contacting cancer cells with cytotoxic T cells and/or immune checkpoint therapy in the presence of monocytes and/or macrophages engineered to decrease the copy number, amount, and/or activity of at least one target listed in Table 1 and/or ii) engineered to increase the copy number, amount, and/or activity of the at least one target listed in Table 2; b) contacting cancer cells with cytotoxic T cells and/or immune checkpoint therapy in the presence of control monocytes and/or macrophages; and c) identifying agents that sensitize cancer cells to cytotoxic T cell-mediated killing and/or immune checkpoint therapy efficacy (such as cell killing) in a) compared to b), is provided.

Generally, the present invention encompasses assays for screening agents, such as test compounds, that bind to, or modulate the activity of, one or more biomarkers encompassed by the present invention (e.g., targets listed in Table 1, Table 2, Examples, etc.). In one embodiment, a method for identifying an agent to modulate an immune response entails determining the ability of the agent to modulate, e.g. enhance or inhibit, one or more targets listed in Table 1 and/or Table 2. Such agents include, without limitation, antibodies, proteins, fusion proteins, small molecules, and nucleic acids.

In some embodiments, a method for identifying an agent which enhances an immune response entails determining the ability of the candidate agent to modulate the one or more biomarkers and further modulate an immune response of interest, such as modulated inflammatory phenotype, cytotoxic T cell activation and/or activity, sensitivity of cancer cells to immune checkpoint therapy, and the like.

In some embodiments, an assay is a cell-free or cell-based assay, comprising contacting one or more biomarkers (e.g., one or more targets listed in Table 1 and/or Table 2), with a test agent, and determining the ability of the test agent to modulate (e.g., upregulate or downregulate) the copy number, amount, and/or activity of the biomarker, such as by measuring direct or indirect parameters as described below.

In some embodiments, an assay is a cell-based assay, such as one comprising contacting (a) a cell of interest (e.g., monocytes and/or macrophages) with a test agent and determining the ability of the test agent to modulate (e.g. upregulate or downregulate) the copy number, amount, and/or activity of the one or more biomarkers, such as binding between the one or more biomarkers and one or more natural binding partners.

Determining the ability of the polypeptides to bind to, or interact with, each other can be accomplished, e.g., by measuring direct binding or by measuring a parameter of immune cell activation.

In another embodiment, an assay is a cell-based assay, comprising contacting a cancer cell with cytotoxic T cells, monocytes and/or macgraophes, and a test agent, and determining the ability of the test agent to modulate the copy number, amount, and/or activity of at least one target listed in Table 1 and/or Table 2, and/or modulated immune responses, such as by measuring direct or indirect parameters as described below.

The methods described above and herein can also be adapted to test one or more agents that are already known to modulate the copy number, amount, and/or activity of one or more biomarkers described herein to confirm modulation of the one or more biomarkers and/or to confirm the effects of the agents on readouts of a desired phenotype, such as modulated immune responses, sensitivity to immune checkpoint blockade, and the like.

In a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ¹H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it can be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies encompassed by the present invention can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

For example, in a direct binding assay, the polypeptides can be coupled with a radioisotope or enzymatic label such that polypeptide interactions and/or activity, such as binding events, can be determined by detecting the labeled protein in a complex. For example, the polypeptides can be labeled with ¹²⁵I, ³⁵S, ¹⁴C, or ³H, either directly or indirectly, and the radioisotope detected by direct counting of radioemmission or by scintillation counting. Alternatively, the polypeptides can be enzymatically labeled with, for example, horseradish peroxidase, alkaline phosphatase, or luciferase, and the enzymatic label detected by determination of conversion of an appropriate substrate to product.

It is also within the scope of the present invention to determine the ability of an agent to modulate a parameter of interest without the labeling of any of the interactants. For example, a microphysiometer can be used to detect interaction between polypeptides without the labeling of polypeptides to be monitored (McConnell et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In some embodiments, determining the ability of the blocking agents (e.g. antibodies, fusion proteins, peptides, or small molecules) to antagonize the interaction between a given set of polypeptides can be accomplished by determining the activity of one or more members of the set of polypeptides. For example, the activity of a protein and/or one or more natural binding partners can be determined by detecting induction of a cellular second messenger (e.g., intracellular signaling), detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the protein and/or the one or more natural binding partners. Determining the ability of the blocking agent to bind to or interact with said polypeptide can be accomplished, for example, by measuring the ability of a compound to modulate immune cell costimulation or inhibition in a proliferation assay, or by interfering with the ability of said polypeptide to bind to antibodies that recognize a portion thereof.

Agents that modulate biomarker amount and/or activity, such as interactions with one or more natural binding partners, can be identified by their ability to inhibit immune cell proliferation, and/or effector function, or to induce anergy, clonal deletion, and/or exhaustion when added to an in vitro assay. For example, cells can be cultured in the presence of an agent that stimulates signal transduction via an activating receptor. A number of recognized readouts of cell activation can be employed to measure, cell proliferation or effector function (e.g., antibody production, cytokine production, phagocytosis) in the presence of the activating agent. The ability of a test agent to block this activation can be readily determined by measuring the ability of the agent to effect a decrease in proliferation or effector function being measured, using techniques known in the art.

For example, agents encompassed by the present invention can be tested for the ability to inhibit or enhance costimulation in a T cell assay, as described in Freeman et al. (2000) J. Exp. Afed. 192:1027 and Latchman et al. (2001) Nat. Immunol. 2:261. CD4+ T cells can be isolated from human PBMCs and stimulated with activating anti-CD3 antibody. Proliferation of T cells can be measured by ³H thymidine incorporation. An assay can be performed with or without CD28 costimulation in the assay. Similar assays can be performed with Jurkat T cells and PHA-blasts from PBMCs.

Alternatively, agents encompassed by the present invention can be tested for the ability to modulate cellular production of cytokines which are produced by or whose production is enhanced or inhibited in immune cells in response to modulation of the one or more biomarkers. Indicative cytokines released by immune cells of interest can be identified by ELISA or by the ability of an antibody which blocks the cytokine to inhibit immune cell proliferation or proliferation of other cell types that is induced by the cytokine. For example, an IL-4 ELISA kit is available from Genzyme (Cambridge Mass.), as is an IL-7 blocking antibody. Blocking antibodies against IL-9 and IL-12 are available from Genetics Institute (Cambridge, Mass.). An in vitro immune cell costimulation assay can also be used in a method for identifying cytokines which can be modulated by modulation of the one or more biomarkers. For example, if a particular activity induced upon costimulation, e.g., immune cell proliferation, cannot be inhibited by addition of blocking antibodies to known cytokines, the activity can result from the action of an unknown cytokine. Following costimulation, this cytokine can be purified from the media by conventional methods and its activity measured by its ability to induce immune cell proliferation. To identify cytokines which can play a role the induction of tolerance, an in vitro T cell costimulation assay as described above can be used. In this case, T cells would be given the primary activation signal and contacted with a selected cytokine, but would not be given the costimulatory signal. After washing and resting the immune cells, the cells would be rechallenged with both a primary activation signal and a costimulatory signal. If the immune cells do not respond (e.g., proliferate or produce cytokines) they have become tolerized and the cytokine has not prevented the induction of tolerance. However, if the immune cells respond, induction of tolerance has been prevented by the cytokine. Those cytokines which are capable of preventing the induction of tolerance can be targeted for blockage in vivo in conjunction with reagents which block B lymphocyte antigens as a more efficient means to induce tolerance in transplant recipients or subjects with autoimmune diseases.

In some embodiments, an assay encompassed by the present invention is a cell-free assay for screening for agents that modulate the interaction between a biomarker and/or one or more natural binding partners, comprising contacting a polypeptide and one or more natural binding partners, or biologically active portion thereof, with a test agent and determining the ability of the test compound to modulate the interaction between the polypeptide and one or more natural binding partners, or biologically active portion thereof. Binding of the test compound can be determined either directly or indirectly as described above. In one embodiment, the assay includes contacting the polypeptide, or biologically active portion thereof, with its binding partner to form an assay mixture, contacting the assay mixture with a test compound, and determining the ability of the test compound to interact with the polypeptide in the assay mixture, wherein determining the ability of the test compound to interact with the polypeptide comprises determining the ability of the test compound to preferentially bind to the polypeptide or biologically active portion thereof, as compared to the binding partner.

In some embodiments, whether for cell-based or cell-free assays, a test agent can further be assayed to determine whether it affects binding and/or activity of the interaction between the polypeptide and the one or more natural binding partners, with other binding partners. Other useful binding analysis methods include the use of real-time Biomolecular Interaction Analysis (BIA) (Sjolander and Urbaniczky (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological polypeptides. Polypeptides of interest can be immobilized on a BIAcore chip and multiple agents (blocking antibodies, fusion proteins, peptides, or small molecules) can be tested for binding to the polypeptide of interest. An example of using the BIA technology is described by Fitz et al. (1997) Oncogene 15:613.

The cell-free assays encompassed by the present invention are amenable to use of both soluble and/or membrane-bound forms of proteins. In the case of cell-free assays in which a membrane-bound form protein is used it can be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In one or more embodiments of the above described assay methods, it can be desirable to immobilize either polypeptides to facilitate separation of complexed from uncomplexed forms of one or both of the proteins, as well as to accommodate automation of the assay. Binding of a test compound to a polypeptide, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-based polypeptide fusion proteins, or glutathione-S-transferase/target fusion proteins, can be adsorbed onto glutathione sepharose beads (Sigma Chemical, St. Louis, Mo.) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of polypeptide binding or activity determined using standard techniques.

In an alternative embodiment, determining the ability of the test compound to modulate the activity of a biomarker of interest (e.g., one or more targets listed in Table 1 and/or Table 2) can be accomplished as described above for cell-based assays, such as by determining the ability of the test compound to modulate the activity of a polypeptide that functions downstream of the polypeptide. For example, levels of second messengers can be determined, the activity of the interactor polypeptide on an appropriate target can be determined, or the binding of the interactor to an appropriate target can be determined as previously described.

The present invention further pertains to novel agents identified by the above-described screening assays. Accordingly, it is within the scope of the present invention to further use an agent identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. Furthermore, the present invention pertains to uses of novel agents identified by the above-described screening assays for treatments as described herein.

3. Diagnostic Uses and Assays

The present invention provides, in part, methods, systems, and code for accurately classifying whether a biological sample is associated with an output of interest, such as monocytes and/or macrophages that are able to have modulated phenotypes according to modulation of one or more biomarkers described herein, a cancer that is likely to respond to cancer therapy (e.g., at least one modulator of one or more targets listed in Table 1 and/or Table 2), and the like. In some embodiments, the present invention is useful for classifying a sample (e.g., from a subject) as associated with or at risk for responding to or not responding to cancer therapy (e.g, at least one modulator of biomarkers listed in Table 1 and/or Table 2) using a statistical algorithm and/or empirical data (e.g., the amount or activity of at least one target listed in Table 1 and/or Table 2). In some embodiments, the present invention encompasses methods of detecting the immune phenotype status of a monocyte and/or macrophage (e.g., M1, Type 1, M2, Type 2, etc.) based on detecting the presence, absence, and/or modulated expression of a biomarker described herein, such as those listed in Table 1, Table 2, the Examples, etc. (e.g., CD53, PSGL1, and/or VSIG4).

An exemplary method for detecting the amount or activity of a biomarker (e.g., one or more targets listed in Table 1 and/or Table 2), and thus useful for classifying whether a sample is likely or unlikely to respond to modulation of inflammatory phenotype, cancer therapy, and the like involves contacting a biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, the method further comprise obtaining a biological sample, such as from a test subject. In some embodiments, at least one agent is used, wherein two, three, four, five, six, seven, eight, nine, ten, or more such agents can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method encompassed by the present invention further comprises sending the sample classification results to a clinician, e.g., an oncologist.

In some embodiments, the diagnosis of a subject is followed by administering to the individual a therapeutically effective amount of a defined treatment based upon the diagnosis.

In some embodiments, the methods further involve obtaining a control biological sample (e.g., biological sample from a subject who does not have a cancer or whose cancer is susceptible to cancer therapy, a biological sample from the subject during remission, or a biological sample from the subject during treatment for developing a cancer progressing despite cancer therapy.

4. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect encompassed by the present invention encompasses diagnostic assays for determining (e.g., detecting) the presence, absence, copy number, amount, and/or activity level of a biomarker described herein, such as those listed in Table 1 and/or Table 2, in the context of a biological sample (e.g., blood, serum, cells, or tissue) to thereby determine whether an individual afflicted with a cancer is likely to respond to cancer therapy (e.g., at least one modulator of biomarkers listed in Table 1 and/or Table 2), whether in an original or recurrent cancer. Such assays can be used for prognostic or predictive purpose to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those listed in Table 1 and/or Table 2. For any predictive medicine analysis, a biomarker of interest, a stratification indicator of interest (e.g., CD11b+ status, CD14+ status, etc.), or any combination thereof, can be analyzed.

Another aspect encompassed by the present invention encompasses monitoring the influence of agents (e.g., drugs, compounds, and small nucleic acid-based molecules) on the expression or activity of a target listed in Table 1 and/or Table 2 and/or inflammatory phenotypes of cells of interest. These and other agents are described in further detail in the following sections.

The skilled artisan will also appreciate that, in certain embodiments, the methods encompassed by the present invention implement a computer program and computer system. For example, a computer program can be used to perform the algorithms described herein. A computer system can also store and manipulate data generated by the methods encompassed by the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part encompassed by the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods encompassed by the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

5. Clinical Efficacy

Clinical efficacy can be measured by any method known in the art. For example, the response to a cancer therapy (e.g., at least one modulator of biomarkers listed in Table 1 and/or Table 2), relates to any response of the cancer, e.g., a tumor, to the therapy, preferably to a change in the number of cancer cells, tumor mass, and/or tumor volume, such as after initiation of neoadjuvant or adjuvant chemotherapy. Tumor response can be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response can also be assessed by caliper measurement or pathological examination of the tumor after biopsy or surgical resection. Response can be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response can be performed early after the onset of neoadjuvant or adjuvant therapy, e.g, after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein can be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular modulator of biomarkers listed in Table 1 and/or Table 2 therapeutic regimen is at least 25%, 30%, 35%, 40%, 45%, 50%, 55%, 60%, 65%, 70%, 75%, 80%, 85%, or more.

Additional criteria for evaluating the response to cancer therapy (e.g., e.g., at least one modulator of biomarkers listed in Table 1 and/or Table 2) are related to “survival,” which includes all of the following: survival until mortality, also known as overall survival (wherein said mortality can be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival can be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular modulator of one or more biomarkers (e.g., targets listed in Table 1 and/or Table 2) can be administered to a population of subjects and the outcome can be correlated to biomarker measurements that were determined prior to administration of any cancer therapy (e.g., e.g., at least one modulator of biomarkers listed in Table 1 and/or Table 2). The outcome measurement can be pathologic response to therapy given in the neoadjuvant setting. Alternatively, outcome measures, such as overall survival and disease-free survival can be monitored over a period of time for subjects following cancer therapy (e.g., at least one modulator of biomarkers listed in Table 1 and/or Table 2) for whom biomarker measurement values are known. In certain embodiments, the same doses of the agent modulating at least one biomarkers listed in Table 1 and/or Table 2 are administered to each subject. In related embodiments, the doses administered are standard doses known in the art for the agent modulating at least one biomarker encompassed by the present invention (e.g., one or more targets listed in Table 1 and/or Table 2). The period of time for which subjects are monitored can vary. For example, subjects can be monitored for at least 2, 4, 6, 8, 10, 12, 14, 16, 18, 20, 25, 30, 35, 40, 45, 50, 55, or 60 months. Biomarker measurement threshold values that correlate to outcome of an cancer therapy (e.g., at least one modulator of biomarkers listed in Table 1 and/or Table 2) can be determined using methods such as those described in the Examples section.

6. Analyzing Biomarker Nucleic Acids and Polypeptides

a. Sample Collection and Preparation

In some embodiments, biomarker amount and/or activity measurement(s) in a sample from a subject is compared to a pre-determined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) can be a biomarker amount and/or activity measurement(s) used to, by way of example only, evaluate a subject that can be selected for treatment, evaluate a response to cancer therapy (e.g., at least one modulator of one or more biomarkers listed in Table 1 and/or Table 2), and/or evaluate a response to a combination cancer therapy (e.g., at least one modulator of one or more biomarkers listed in Table 1 and/or Table 2 in combination of at least one immunotherapy). A pre-determined biomarker amount and/or activity measurement(s) can be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject can affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of cancer therapy. Post-treatment biomarker measurement can be made at any time after initiation of cancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of cancer therapy, and even longer toward indefinitely for continued monitoring. Treatment can comprise cancer therapy, such as a therapeutic regimen comprising one or more modulators of at least one target listed in Table 1 and/or Table 2, either alone or in combination with other cancer agents, such as immune checkpoint inhibitors.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments encompassed by the present invention the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cut-off values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process can isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters can further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis can have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention can include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

b. Analyzing Biomarker Nucleic Acids and Poypeptides

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

c. Methods for Detection of Copy Number and/or Genomic Nucleic Acid Mutations

Methods of evaluating the copy number and/or genomic nucleic acid status (e.g. mutations) of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. In some embodiments, the increased copy number of at least one target listed in Table 1, and/or the decreased copy number of at least one target listed in Table 2 is predictive of poor outcome of cancer therapy (e.g., at least one modulator of biomarkers listed in Table 1 and/or Table 2). A copy number of at least 3, 4, 5, 6, 7, 8, 9, or 10 of at least one target listed in Table 1 and/or Table 2 is predictive of likely responsive to cancer therapy (e.g., at least one modulator of biomarkers listed in Table 1 and/or Table 2).

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g., membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot can be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a pre-determined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids can comprise cDNAs, genomic DNAs, oligonucleotides (e.g., to detect single nucleotide polymorphisms) and the like. Array-based CGH can also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3:1227-1234; Pinkel (1988) Proc. Natl. Acad. Sci. U.S.A. 85:9138-9142; EP Pat. Publ. No. 430,402; Methods in Molecular Biology, Vol. 33: In situ Hybridization Protocols, Choo, ed., Humana Press, Totowa, N.J. (1994), etc.). In another embodiment, the hybridization protocol of Pinkel, et al. (1998) Nat. Genet. 20:207-211, or of Kallioniemi (1992) Proc. Natl. Acad. Sci. U.S.A. 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g., healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided in Innis, et at. (1990) PCR Protocols, A Guide to Methods and Applications, Academic Press, Inc. N.Y.). Measurement of DNA copy number at microsatellite loci using quantitative PCR analysis is described in Ginzonger et al. (2000) Cancer Res. 60:5405-5409. The known nucleic acid sequence for the genes is sufficient to enable one of skill in the art to routinely select primers to amplify any portion of the gene. Fluorogenic quantitative PCR can also be used in the methods encompassed by the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4:560, Landegren, et al. (1988) Science 241:1077, and Barringer et al. (1990) Gene 89:117), transcription amplification (Kwoh et al. (1989) Proc. Natl. Acad. Sci. U.S.A. 86:1173), self-sustained sequence replication (Guatelli et al. (1990) Proc. Natl. Acad. Sci. U.S.A. 87:1874), dot PCR, and linker adapter PCR, etc.

Loss of heterozygosity (LOH) and major copy proportion (MCP) mapping (Wang et al. (2004) Cancer Res. 64:64-71; Seymour et al. (1994) Cancer Res. 54:2761-2764; Hahn et at. (1995) Cancer Res. 55:4670-4675; Kimura et al. (1996) Genes Chromosomes Cancer 17:88-93: Li et al. (2008) MBC Bioinform. 9:204-219) can also be used to identify regions of amplification or deletion.

d. Methods for Detection of Biomarker Nucleic Acid Expression

Biomarker expression can be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278:1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it can be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells can quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al. (1979) Biochem. 18:5294-5299). RNA from single cells can be obtained as described in methods for preparing cDNA libraries from single cells, such as those described in Dulac (1998) Curr. Top. Dev. Biol. 36:245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides can be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences.

Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope encompassed by the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by Marshall et al. (1994) PCR Methods Appls. 4:80-84. Real-time PCR can also be used.

Other known amplification methods which can be utilized herein include but are not limited to the so-called “NASBA” or “3SR” technique described in Guatelli et al. (1990) Proc. Natl. Acad. Sci. U.S.A. 87:1874-1878 and also described in Compton et al. (1991) Nature 350:91-92: Q-beta amplification as described in EP Pat. Publ. No. 4544610; strand displacement amplification (as described in Walker et al. (1996) Clin. Chem. 42:9-13 and EP Pat. Publ. No. 684315: target mediated amplification, as described by PCT Publ. No. WO 93/22461; PCR; ligase chain reaction (LCR) (see, e.g., Wu and Wallace (1989) Genomics 4:560, Landegren et al. (1988) Science 241:1077); self-sustained sequence replication (SSR) (see, e.g., Guatelli et al. (1990) Proc. Nat. Acad. Sci. U.S.A. 87:1874); and transcription amplification (see, e.g., Kwoh et al. (1989) Proc. Nat. Acad. Sci. U.S.A. 86:1173).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with hematoxylin to demonstrate the histological composition of the sample, and dark field imaging with a suitable light filter shows the developed emulsion. Non-radioactive labels such as digoxigenin can also be used.

Alternatively, mRNA expression can be detected on a DNA array, chip or a microarray. Labeled nucleic acids of a test sample obtained from a subject can be hybridized to a solid surface comprising biomarker DNA. Positive hybridization signal is obtained with the sample containing biomarker transcripts. Methods of preparing DNA arrays and their use are well-known in the art (see, e.g., U.S. Pat. Numbers 6,618,6796; 6,379,897; 6,664,377; 6,451,536; and 6,548,257; U.S. Pat. Publ. No. 2003/0157485; and Schena el al. (1995) Science 20:467-470; Gerhold el al. (1999) Trends Biochem. Sci. 24:168-173; and Lennon et al. (2000) Drug Discovery Today 5:59-65). Serial Analysis of Gene Expression (SAGE) can also be performed (See for example U.S. Pat. Publ. No. 2003/0215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes can be as short as is required to differentially recognize marker mRNA transcripts, and can be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes can be any that is appropriate, such as the use of radioisotopes, for example, ³²P and ³⁵S. Labeling with radioisotopes can be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

e. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to a modulator of T cell mediated cytotoxicity alone or in combination with an immunotherapy treatment. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991). Preferred are binder-ligand immunoassay methods including reacting antibodies with an epitope or epitopes and competitively displacing a labeled polypeptide or derivative thereof.

For example, ELISA and RIA procedures can be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies can be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited, but can be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect biomarker protein according to a practitioner's preference based upon the present disclosure. One such technique is Western blotting (Towbin et al. (1979) Proc. Nat. Acad. Sci. U.S.A. 76:4350), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection can also be used.

Immunohistochemistry can be used to detect expression of biomarker protein, e.g., in a biopsy sample. A suitable antibody is brought into contact with, for example, a thin layer of cells, washed, and then contacted with a second, labeled antibody. Labeling can be by fluorescent markers, enzymes, such as peroxidase, avidin, or radiolabeling. The assay is scored visually, using microscopy.

Anti-biomarker protein antibodies, such as intrabodies, can also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose can be any that do not substantially interfere with the antibody binding, but which allow external detection. Suitable markers can include those that can be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which can be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that can be used to detect biomarker protein include any antibody, whether natural or synthetic, full length or a fragment thereof, monoclonal or polyclonal, that binds sufficiently strongly and specifically to the biomarker protein to be detected. An antibody can have a K_{d} of at most about 10⁻⁶M, 10⁻⁷M, 10⁻⁸M, 10⁻⁹M, 10⁻¹⁰M, 10⁻¹¹M, or 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody can bind preferentially to the biomarker protein relative to other proteins, such as related proteins.

Antibodies are commercially available or can be prepared according to methods known in the art.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

f. Method for Detection of Biomarker Structural Alterations

The following illustrative methods can be used to identify the presence of a structural alteration in a biomarker nucleic acid and/or biomarker polypeptide molecule in order to, for example, identify sequences or agents that affect T cell mediated killing of cancer cells.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et a. (1988) Science 241:1077-1080; and Nakazawa et al. (1994) Proc. Natl. Acad. Sci. USA. 91:360-364), the latter of which can be particularly useful for detecting point mutations in a biomarker nucleic acid such as a biomarker gene (see Abravaya et al. (1995) Nucl. Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR can be desirable to use as a preliminary amplification step in conjunction with any of the techniques used for detecting mutations described herein.

Alternative amplification methods include: self-sustained sequence replication (Guatelli et al. (1990) Proc. Natl. Acad Sci. U.S.A. 87:1874-1878), transcriptional amplification system (Kwoh et al. (1989) Proc. Natl. Acad. Sci. U.S.A. 86:1173-1177), Q-Beta Replicase (Lizardi et al. (1988) Biotechntol. 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin et al. (1996) Hum. Autat. 7:244-255, Kozal et al. (1996) Nat. Med 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. U.S.A. 74:560 or Sanger (1977) Proc. Natl. Acad Sci. U.S.A. 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT Publ. No. WO 94/16101; Cohen et al. (1996) Adv. Chromatogr. 36:127-162; and Griffin et al. (1993)Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. U.S.A. 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039).

In other embodiments, alterations in electrophoretic mobility can be used to identify mutations in biomarker genes. For example, single strand conformation polymorphism (SSCP) can be used to detect differences in electrophoretic mobility between mutant and wild type nucleic acids (Orita et al. (1989) Proc Natl. Acad. Sci U.S.A. 86:2766; Cotton (1993) Mutat. Res. 285:125-144; Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers can be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. U.S.A. 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it can be desirable to introduce a novel restriction site in the region of the mutation to create cleavage-based detection (Gasparini et al. (1992) Mol. Cell Probes 6:1). It is anticipated that in certain embodiments amplification can also be performed using Taq ligase for amplification (Barany (1991) Proc. Natl. Acad. Sci U.S.A. 88:189). In such cases, ligation will occur only if there is a perfect match at the 3′ end of the 5′ sequence making it possible to detect the presence of a known mutation at a specific site by looking for the presence or absence of amplification.

### VI. Compositions, Including Formulations and Pharmaceutical Compositions

Compositions comprising agents encompassed by the present invention are contemplated without limitation. For example, nucleic acid-based compositions (e.g., messenger RNA (mRNA), cDNA, siRNA, antisense nucleic acids, oligonucleotides, ribozymes, DNAzymes, aptamers, nucleic acid decoys, nucleic acid chimeras, triple helical structures, etc.), protein-based compositions, cell-based componsitions, as well as variants, modifications, and engineered versions thereof, are contemplated for use in the methods described herein as well as compositions per se. In some embodiments, siRNA molecules having a sense strande nucleic acid sequence and an antisense strand nucleic acid sequence, each selected from sequences described herein, as well as sequence variant and/or chemically modified versions thereof, are encompassed by the present invention and are described in detail above. In some embodiments, cells modified as described herein, such as monocytes and/or macrophages having a modulated inflammatory phenotype.

Such compositions can be comprised within pharmaceutical compositions and/or formulations. Such compositions can be prepared by any method known or hereafter developed in the art of pharmacology. In general, such preparatory methods include the step of bringing the agent, such as an active ingredient, into association with an excipient and/or one or more other accessory ingredients, and then, if necessary and/or desirable, dividing, shaping and/or packaging the product into a desired single- or multi-dose unit. As used herein, the term “active ingredient” refers to any chemical and biological substance that has a physiological effect in human or in animals, when exposed to it. In the context encompassed by the present invention, the active ingredient in the formulations can be any of the agents that modulate a biomarker encompassed by the present invention (e.g., at least one target listed in Table 1 and/or Table 2).

### 1. Composition Preparation

A composition in accordance with the invention can be prepared, packaged, and/or sold in bulk, as a single unit dose, and/or as a plurality of single unit doses. As used herein, a “unit dose” is discrete amount of the pharmaceutical composition comprising a pre-determined amount of the active ingredient. The amount of the active ingredient is generally equal to the dosage of the active ingredient which would be administered to a subject and/or a convenient fraction of such a dosage such as, for example, one-half or one-third of such a dosage.

The term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutical compositions encompassed by the present invention can be presented as anhydrous pharmaceutical formulations and dosage forms, liquid pharmaceutical formulations, solid pharmaceutical formulations, vaccines, and the like. Suitable liquid preparations can include, but are not limited to, isotonic aqueous solutions, suspensions, emulsions, or viscous compositions that are buffered to a selected pH.

As described in detail below, the agents and other compositions encompassed by the present invention can be specially formulated for administration in solid or liquid form, including those adapted for various routes of administration, such as (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. Any appropriate form factor for an agent or composition described herein, such as, but not limited to, tablets, capsules, liquid syrups, soft gels, suppositories, and enemas, is contemplated.

Pharmaceutical compositions encompassed by the present invention can be presented as discrete dosage forms, such as capsules, sachets, or tablets, or liquids or aerosol sprays each containing a pre-determined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered peptide or peptidomimetic moistened with an inert liquid diluent.

Tablets, and other solid dosage forms, such as dragees, capsules, pills and granules, can optionally be scored or prepared with coatings and shells, such as enteric coatings and other coatings well-known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions, which can be dissolved in sterile water, or some other sterile injectable medium immediately before use. These compositions can also optionally contain opacifying agents and can be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more excipients.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents. Suspensions, in addition to the active agent can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration can be presented as a suppository, which can be prepared by mixing one or more agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component can be mixed under sterile conditions with a pharmaceutically-acceptable carrier, and with any preservatives, buffers, or propellants which can be required.

The ointments, pastes, creams and gels can contain, in addition to an agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Agent can be administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of an agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

In some embodiments, pharmaceutical compositions encompassed by the present invention are formulated in parenteral dosage forms. The parenteral formulations can be aqueous solutions containing carriers or excipients such as salts, carbohydrates and buffering agents (e.g., at a pH of from 3 to 9), or sterile non-aqueous solutions, or dried forms which can be used in conjunction with a suitable vehicle such as sterile, pyrogen-free water. For example, an aqueous solution of the therapeutic agents encompassed by the present invention comprises an isotonic saline, 5% glucose or other pharmaceutically acceptable liquid carriers such as liquid alcohols, glycols, esters, and amides, for example, as disclosed in U.S. Pat. No. 7,910,594. In another example, an aqueous solution of the therapeutic agents encompassed by the present invention comprises a phosphate buffered formulation (pH 7.4) for intravenous administration as disclosed in PCT Publ. No. WO 2011/014821. The parenteral dosage form can be in the form of a reconstitutable lyophilizate comprising the dose of the therapeutic agents encompassed by the present invention. Any prolonged release dosage forms known in the art can be utilized such as, for example, the biodegradable carbohydrate matrices described in U.S. Pat. Nos. 4,713,249, 5,266,333; and 5,417,982, or, alternatively, a slow pump (e.g., an osmotic pump) can be used. The preparation of parenteral formulations under sterile conditions, for example, by lyophilization under sterile conditions, can readily be accomplished using standard pharmaceutical techniques well-known to those skilled in the art. The solubility of a therapeutic agent encompassed by the present invention used in the preparation of a parenteral formulation can be increased by the use of appropriate formulation techniques, such as the incorporation of solubility-enhancing agents. Formulations for parenteral administration can comprise one or more agents in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain antioxidants, buffers, bacteriostats, solutes which render the formulation isotonic with the blood of the intended recipient or suspending or thickening agents.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the agents encompassed by the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention can be determined by the methods encompassed by the present invention so as to obtain an amount of the active ingredient, which is effective to achieve the desired therapeutic response for a particular subject, composition, and mode of administration, without being toxic to the subject.

In some embodiments, pharmaceutical compositions encompassed by the present invention can be formulated for controlled release and/or targeted delivery. As used herein, “controlled release” refers to a pharmaceutical composition or compound release profile that conforms to a particular pattern of release to effect a therapeutic outcome. In one embodiment, the compositions encompassed by the present invention can be encapsulated into a delivery agent described herein and/or known in the art for controlled release and/or targeted delivery. As used herein, the term “encapsulate” means to enclose, surround or encase. As it relates to the formulation encompassed by the present invention, encapsulation can be substantial, complete or partial. The term “substantially encapsulated” means that at least greater than 50, 60, 70, 80, 85, 90, 95, 96, 97, 98, 99, 99.9, 99.9 or greater than 99.999% of a therapeutic agent encompassed by the present invention can be enclosed, surrounded or encased within the particle. The term “partially encapsulation” means that less than 10, 10, 20, 30, 40 50 or less of the conjugate encompassed by the present invention can be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound encompassed by the present invention are encapsulated in the formulation.

In some embodiments, such formulations can also be constructed or compositions altered such that they passively or actively are directed to different cell types in vivo, including but not limited to monocytes, macrophages, and other immune cells (e.g., dendritic cells, antigen presenting cells, T lymphocytes, B lymphocytes, and natural killer cells), cancer cells and the like. Formulations can also be selectively targeted through expression of different ligands on their surface as exemplified by, but not limited by, folate, transferrin, N-acetylgalactosamine (GalNAc), and antibody targeted approaches.

2. Excipients

The pharmaceutical compositions encompassed by the present invention can be formulated using one or more excipients to: (1) increase stability; (2) permit the sustained or delayed release (e.g., from a depot formulation); (3) alter the biodistribution (e.g., target an agent to a specific tissue or cell type); (4) alter the release profile of the agent in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients encompassed by the present invention can also include, without limitation, lipidoids, liposomes, lipid nanoparticles, polymers, lipoplexes, core-shell nanoparticles, peptides, proteins, hyaluronidase, nanoparticle mimics and combinations thereof.

The term “pharmaceutically acceptable carrier” or “pharmaceutically acceptable excipient” is intended to include any and all solvents, dispersion media, diluents or other liquid vehicles, dispersion or suspension agents, surface active agents, isotonic agents, thickening or emulsifying agents, disintegrating agents, preservatives, buffering agents, solid binders, lubricants, oils, coatings, antibacterial and antifungal agents, absorption delaying agents, and the like, as suited to the particular dosage form desired. Remington's The Science and Practice of Pharmacy, 21st Edition, A. R. Gennaro (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention. Supplementary active ingredients can also be incorporated into the described compositions.

In some embodiments, a pharmaceutically acceptable excipient is at least 95%, at least 96%, at least 97%, at least 98%, at least 99%, at least 99.5%, or at least 99.9% or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, mannitol,); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol, etc.; and combinations thereof.

Exemplary preservatives can include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP, methylparaben, GERMALL®115, GERMABEN® II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Pharmaceutical formulations can also comprise pharmaceutically acceptable salts. The term “pharmaceutically acceptable salt” refers to salts derived from a variety of organic and inorganic counter ions known in the art (see, e.g., Berge et al. (1977) J. Pharm. Sci. 66:1-19). These salts can be prepared in situ during the final isolation and purification of the agents, or by separately reacting a purified agent in its free base form with a suitable organic or inorganic acid, and isolating the salt thus formed. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In some embodiments, agents encompassed by the present invention can contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression. These salts can likewise be prepared in situ during the final isolation and purification of the agents, or by separately reacting the purified agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

The term “co-crystal” refers to a molecular complex derived from a number of co-crystal formers known in the art. Unlike a salt, a co-crystal typically does not involve hydrogen transfer between the co-crystal and the drug, and instead involves intermolecular interactions, such as hydrogen bonding, aromatic ring stacking, or dispersive forces, between the co-crystal former and the drug in the crystal structure.

Exemplary surfactants which can be used to form pharmaceutical compositions and dosage forms encompassed by the present invention include, but are not limited to, hydrophilic surfactants, lipophilic surfactants, and mixtures thereof. That is, a mixture of hydrophilic surfactants can be employed, a mixture of lipophilic surfactants can be employed, or a mixture of at least one hydrophilic surfactant and at least one lipophilic surfactant can be employed. Hydrophilic surfactants can be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof. Ionic surfactants can include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants can be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants can include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterifcation products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol can be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants can include, but are not limited to, fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers: lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

Solubilizers can be included in the present formulations to ensure good solubilization and/or dissolution of the agent (e.g., a chemical compound) encompassed by the present invention and to minimize precipitation of the drug modality encompassed by the present invention. This can be especially important for compositions for non-oral use, such as compositions for injection. A solubilizer can also be added to increase the solubility of the hydrophilic drug and/or other components, such as surfactants, or to maintain the composition as a stable or homogeneous solution or dispersion. Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, [symbol] -caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon-caprolactone and isomers thereof, j-valerolactone and isomers thereof, ü-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers can also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

Pharmaceutically acceptable additives can be included in a formulation as needed. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base can be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example can include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### 3. Lipid-Based Formulations

In some embodiments, lipid-based formulations are used. Accordingly, provided herein are lipid-based formulations comprising a composition as described herein and one or more lipids. In some embodiments, the lipidisalipid particle or amphiphilic compound. The lipid can be neutral, anionic, or cationic at physiologic pH.

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), for example, between 0.1-30 (weight lipid/w polymer). Phospholipids which can be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-γ-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) can also be used.

In some embodiments, lipid-based particles are used. The term “lipid particles” refers to liposomes, lipid micelles, solid lipid particles, lipoplexes, lipid nanoparticles (LNPs), or lipid-stabilized polymeric particles, composed of one or a mixture of different biocompatible lipids, e.g., at least one or more cationic lipids and/or one or more neutral lipids and/or polyethylene glycol (PEG)-lipids.

The particle can be a lipid micelle. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a solid lipid particle. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as 1-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

Liposomes are artificially-prepared vesicles which can primarily be composed of a lipid bilayer and can be used as a delivery vehicle for the administration of nutrients and pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations.

The formation of liposomes can depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein can include, without limitation, liposomes such as those formed from 1,2-dioleyloxy-NN-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (e.g., as described in U.S. Pat. Publ. No. 2010/0324120).

In one embodiment, the compositions encompassed by the present invention can be formulated in a lipid-polycation complex. The formation of the lipid-polycation complex can be accomplished by methods known in the art and/or as described in U.S. Pat. Publ. No. 2012/0178702. As a non-limiting example, the polycation can include a cationic peptide or a polypeptide such as, but not limited to, polylysine, polyornithine and/or polyarginine and the cationic peptides described in PCT Publ. No. WO 2012/013326. In another embodiment, the compositions encompassed by the present invention can be formulated in a lipid-polycation complex which can further include a neutral lipid such as, but not limited to, cholesterol or dioleoyl phosphatidylethanolamine (DOPE). The liposome formulation can be influenced by, but not limited to, the selection of the cationic lipid component, the degree of cationic lipid saturation, the nature of the PEGylation, ratio of all components and biophysical parameters such as size.

In some embodiments, the lipid particle is a lipid nanoparticle (LNP). The term “lipid nanoparticle (LNP)” refers to lipid-based particles in the submicron range which include one or more lipid components as described herein. LNPs can have structural characteristics of liposomes and/or have alternative non-bilayer types of structures, which can be used to systemically deliver nucleic acid based drugs, including, for example, siRNA molecules complementary to the nucleic acid sequence of mRNA transcribed from at least one biomarker (e.g., at least one target listed in Table 1 and/or Table 2) described herein. In some embodiments, the LNP formulation comprises one or more cationic lipids. Cationic lipids are lipids that carry a net positive charge at any physiological pH. In certain particular embodiments, the LNP comprises a lipidoid as described herein. The positive charge is useful for association with negatively charged therapeutic agents, such as siRNA molecules.

In certain embodiments, a lipid nanoparticle comprises one or more lipids and a composition as described herein. In certain particular embodiments, a composition as described herein is encapsulated within a lipid nanoparticle.

In some embodiments, the sizes and charge ratios and other physical properties (e.g., membrane fluidity) of LNPs are optimized for increased cell transfection and delivery.

Lipid or lipidoid particles can comprise, for example, cationic lipids, neutral lipids, amino acid- or peptide-based lipids, polyethylene glycol (PEG)-lipids, e.g., lipids with PEG chains such as hydrogenated soybean phosphatidylcholine (HSPC), cholesterol (CHE), 1, 2-distearoyl-glycero-3-phosphoethanolamine-N-[methoxy (PEG)-2000] (DSPE-PEG2000), 1, 2-distearoyl-sn-glycero-3-phosphoethanolamine-N-[methoxy (PEG)-2000] modified with a maleimidic group in the distal end of the chain 1, 2-distearoyl-sn-glycero-3-phosphoethanolamine-N-[maleimide (PEG)-2000], DSPE-PEG2000-MAL, 1,2-dimyristoyl-sn-glycero-3-phosphoethanolamine-N-[methoxy(polyethylene glycol)-550](DMPE-PEG550), 1, 2-dioleoyl-1-3-trimethylammonium propane (DOTAP), and those with a glycerol backbone e.g., DMG-PEG, DSG-PEG (DMG-PEG2000) etc. As used herein, a liposome is a structure comprising lipid-containing membranes enclosing an aqueous interior. For example, lipid-based formulations can be used to deliver nucleic acid agents of the present invention, e.g., siRNAs, miRNAs, oligonucleotides, modified mRNAs and other types of nucleic acid molecules.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

A number of cationic lipids, and methods for making them, are described in, for example, U.S. Pat. Nos. 5,830,430; 6,056,938; 7,893,302; 7,404,969; 8,034,376; 8,283,333; and 8,642,076, as well as PCT Publ. Numbers WO 2010/054406, WO 2010/054401, WO 2010/054405, WO 2010/054384, WO 2012/040184, WO 2011/153120, WO 2011/149733, WO 2011/090965, WO 2011/043913, WO 2011/022460, WO 2012/061259, WO 2012/054365, WO 2012/044638, WO 2010/080724, WO 2010/21865, and WO 2008/103276.

The term “cationic lipid” is meant to include those lipids having one or two fatty acid or fatty aliphatic chains and an amino head group (including an alkylamino or dialkylamino group) that can be protonated to form a cationic lipid at physiological pH, which consist of a positively charged headgroup and a hydrophobic tail. The positively charged headgroup can serve to electrostatically bind the negatively charged siRNA molecule, while the hydrophobic tail leads to self-assembly into lipophilic particles. Examples of cationic lipids can include, but are not limited to: DLin-K-DMA, DLinDMA, DLinDAP, DLin-K-C2-DMA, DLin-K2-DMA, DOTAP, DMRIE, DORIE, DOTMA, DDAB, Ethyl PC, multivalent cationic lipid and DC-cholesterol, DODA, DODMA, DSDMA, DOTMA, DDAB, DODAP, DOTAP, DOTAP-C1, DC-Chol, DMRIE, DOSPA, DOGS, DOPE, CLinDMA, CpLinDMA, DMOBA, DOcarbDAP, DLincarbDAP, DLinCDAP. A number of these lipids and related analogs have been described in U.S. Pat. Publ. Numbers 2006/0083780 and 2006/0240554; and U.S. Pat. Nos. 5,208,036; 5,264,618; 5,279,833; 5,283,185; 5,753,613 and 5,785,992. Cationic lipids can also be a lipofectin (see, e.g., U.S. Pat. No. 5,705,188), such as Lipofectamine®, Lipofectamine 2000®, Lipofectamine 3000®, RNAiMAX®, and the like.

Other cationic lipids, which carry a net positive charge at about physiological pH, can be used in the lipid particles of the present invention, including, but not limited to, N,N-dioleyl-N,N-dimethylammonium chloride (DODAC), dioctadecyldimethylammonium (DODMA), distearyldimethylammonium (DSDMA), N-(1-(2,3-dioleyloxy)propyl)-N,N,N-trimethylammonium chloride (DOTMA), N,N-distearyl-N,N-dimethylammonium bromide (DDAB), 1,2-dioleoyl-3-dimethylammonium propane (DODAP), N-(1-(2,3-dioleoyloxy)propyl)-N,N,N-trimethylammonium chloride (DOTAP), 1,2-Dioleyloxy-3-trimethylaminopropane chloride salt (DOTAP.Cl), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS). 1,2-dileovl-sn-3-phosphoethanolamine (DOPE, which carries a positive charge at physiological pH but at acidic pH), 3-dimethylamino-2-(cholest-5-en-3-beta-oxybutan-4-oxy)-1-(cis,cis-9,12-octadecadienoxy)propane (CLinDMA), 2-[5′-(cholest-5-en-3β-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-Dilinoleoylcarbamyl-3-dimethylaminopropane (DLinCDAP), and mixtures thereof. A number of these lipids and related analogs have been described in U.S. patent application publication NOs. 2006/0083780 and 2006/0240554; U.S. Pat. Nos. 5,208,036; 5,264,618; 5,279,833; 5,283,185; 5,753,613 and 5,785,992.

Suitable additional cationic lipids can also include, but are not limited to, N-[1-(2,3-dioleoyloxy)propyl]-N,N,N-trimethyl ammonium salts, also referenced as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), 0-alanyl cholesterol, cetyl trimethyl ammonium bromide (CTAB), diCia-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Cationic lipids can also be ionizable cationic lipids. Suitable ionizable cationic lipids for use in formulating a composition described herein include lipids described in WO2015/074805. Other suitable ionizable cationic lipids suitable for formulating a composition of the present invention can include those described in US 2015/0239834.

In some embodiments, symmetric or asymmetric or ionizable cationic lipids can be used in a nanoparticle or lipid formulation. Such lipids are disclosed in, for example, U.S. Patent application publication Nos. 2015/0239926, 2015/0239834 and 2015/0141678 and PCT Publ. No. WO 2015/074805.

Additionally, a number of commercial preparations of cationic lipids can be used, such as LIPOFECTIN® (including DOTMA and DOPE, available from GIBCO/BRL), LIPOFECTAMINE® (comprising DOSPA and DOPE, available from GIBCO/BRL), TRANSFECTIN® (from Bio-Rad Laboratories, Inc.) and siPORT NEOFX® (from Applied Biosystems).

Cationic lipids can also be modified cationic lipids suitable for cellular delivery of compositions comprising agents described herein, such as siRNA molecules (see, for example, those described in U.S. Pat. Publ. No. 2013/0323269); cationic glycerol derivatives, and polycationic molecules, such as polylysine (PCT Publ. No. WO 97/30731), cationic group including one or more biodegradable groups (U.S. Pat. Publ. No. 2013/0195920).

In some embodiments, the ionizable lipid can be ionizable amino lipids described in WO 2015/074805 or US 2015/0239834.

In certain embodiments, a composition described herein further comprises an aminoalcohol lipidoid as described in WO 2010/053572. In certain embodiments, the lipidoid compound is selected from Formulae (I)-(V):

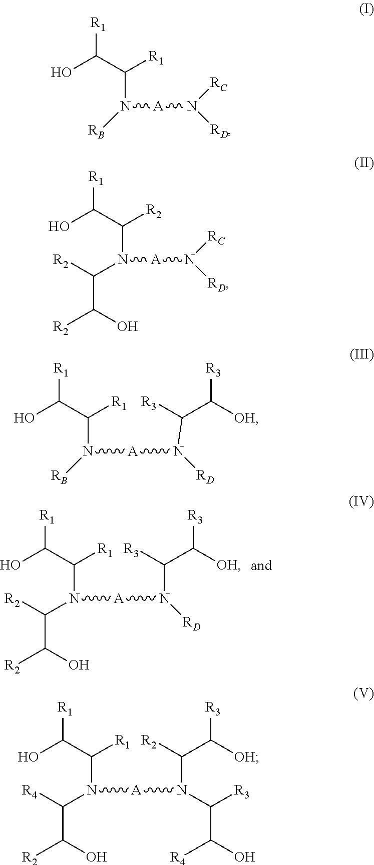

and pharmaceutically acceptable salts thereof, wherein:

A is a substituted or unsubstituted, branched or unbranched, cyclic or acyclic C₂₋₂₀ alkylene, optionally interrupted by 1 or more heteroatoms independently selected from O, S and N, or A is a substituted or unsubstituted, saturated or unsaturated 4-6-membered ring;

R₁ is hydrogen, a substituted, unsubstituted, branched or unbranched C₁₋₂₀-aliphatic or a substituted, unsubstituted, branched or unbranched C₁₋₂₀ heteroaliphatic, wherein at least one occurrence of R₁ is hydrogen; R_{B}, R_{C}, and R_{D} are, independently, hydrogen, a substituted, unsubstituted, branched or unbranched C₁₋₂₀-aliphatic, or a substituted, unsubstituted, branched or unbranched C₁₋₂₀-heteroaliphatic or —CH₂CH(OH)R_{E};

R_{B} and R_{D} together can optionally form a cyclic structure;

R_{C} and R_{D} together can optionally form a cyclic structure; and
R_{E} is a substituted, unsubstituted, branched or unbranched C₁₋₂₀ aliphatic or a substituted, unsubstituted, branched or unbranched C₁₋₂₀ heteroaliphatic.

In certain particular embodiments, the lipidoid is of Formula (VI):

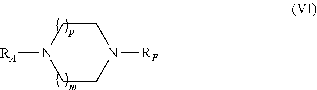

or a pharmaceutically acceptable salt thereof, wherein:

p is an integer between 1 and 3, inclusive;

m is an integer between 1 and 3, inclusive;

R_{A} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

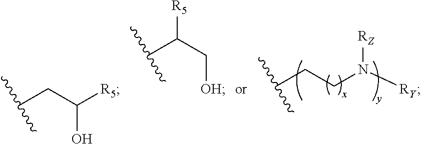

R_{F} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

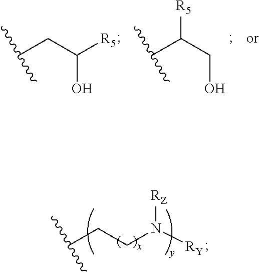

each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;

wherein, at least one of R_{A}, R_{F}, R_{Y}, and R_{Z} is

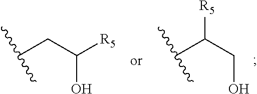

each occurrence of x is an integer between 1 and 10, inclusive;

each occurrence of y is an integer between 1 and 10, inclusive;

each occurrence of R_{Y} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

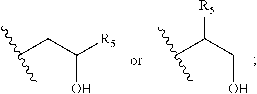

each occurrence of R_{Z} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

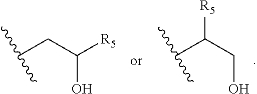

In certain embodiments of Formula (VI), p is 1. In certain embodiments, m is 1. In certain embodiments, p and m are both 1. In certain embodiments, R_{F} is

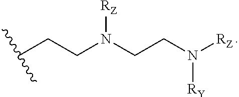

In certain embodiments, R_{A} is

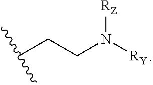

In certain embodiments, the composition comprises an aminoalcohol lipidoid selected from C14-120, C16-120, C14-98, C14-113, C14-96, C12-200, C12-205, C16-96, C12-111, and C12-210 (see U.S. Pat. No. 8,450,298 and PCT Publ. No. WO 2010/053572, referenced above).

In certain particular embodiments, the aminoalcohol lipidoid is C12-200:

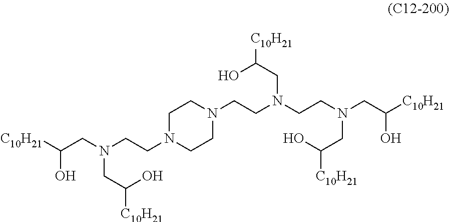

In certain particular embodiments, the lipidoid is of Formula (VII):

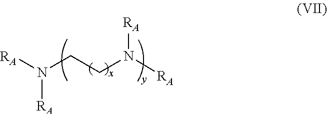

or a pharmaceutically acceptable salt thereof, wherein:

each occurrence of R_{A} is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

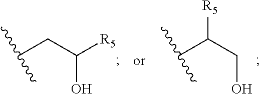

wherein at least one R_{A} is

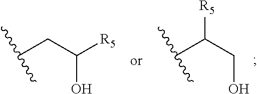

each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;

each occurrence of x is an integer between 1 and 10, inclusive; and

each occurrence of y is an integer between 1 and 10, inclusive.

In certain embodiments, a composition described herein further comprises an amine-containing lipidoid as described in WO 2014/028847.

In certain embodiments, the amine-containing lipidoid is of Formula (VIII):

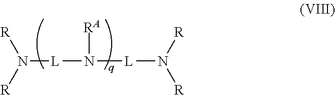

or a pharmaceutically acceptable salt thereof, wherein:

each L is, independently, branched or unbranched C₁₋₆ alkylene, wherein L is optionally substituted with one or more fluorine radicals;

each R^{A} is, independently, branched or unbranched C₁₋₆ alkyl, C₃₋₇ cycloalkyl, or branched or unbranched C₄₋₁₂ cycloalkylalkyl, wherein R^{A} is optionally substituted with one or more fluorine radicals;

each R is, independently, hydrogen or —CH₂CH₂C(═O)OR^{B};

each R^{B} is, independently, C₁₀₋₁₄ alkyl, wherein R^{B} is optionally substituted with one or more fluorine radicals; and

q is 1, 2, or 3;

provided that at least three R groups are —CH₂CH₂C(═O)OR^{B};

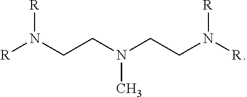

provided that the compound is not CH₃

In certain embodiments, a composition described herein further comprises a polyamine-fatty acid derived lipidoid as described in WO 2016/004202.

In certain embodiments, the amine-containing lipidoid is of Formula (IX):

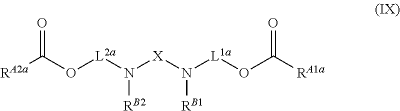

or a pharmaceutically acceptable salt, wherein:

X is substituted or unsubstituted alkylene, substituted or unsubstituted alkenylene, substituted or unsubstituted alkynylene, substituted or unsubstituted heteroalkylene, substituted or unsubstituted heteroalkenylene, substituted or unsubstituted heteroalkynylene, substituted or unsubstituted carbocyclylene, substituted or unsubstituted heterocyclylene, substituted or unsubstituted arylene, substituted or unsubstituted heteroarylene, a divalent moiety of the formula:

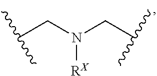

or a combination thereof, wherein each instance of R^{X} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group, or a moiety of the formula:

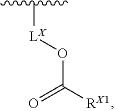

or R^{B1} and an instance of R^{X} are joined to forma substituted or unsubstituted, heterocyclic ring or a substituted or unsubstituted, heteroaryl ring, or R^{B2} and an instance of R^{X} are joined to form a substituted or unsubstituted, heterocyclic ring or a substituted or unsubstituted, heteroaryl ring, wherein:

each instance of L^{X} is independently substituted or unsubstituted alkylene or substituted or unsubstituted heteroalkylene; and

each instance of R^{X1} is independently substituted or unsubstituted, C₄₋₃₀ alkyl, substituted or unsubstituted, C₄₋₃₀ alkenyl, or substituted or unsubstituted, C₄₋₃₀ alkynyl;

L^{1a} is substituted or unsubstituted alkylene or substituted or unsubstituted heteroalkylene;

R^{A1a} is substituted or unsubstituted, C₄₋₃₀ alkyl, substituted or unsubstituted, C₄₋₃₀ alkenyl, or substituted or unsubstituted, C₄₋₃₀ alkynyl;

R^{B1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group, or a moiety of the formula:

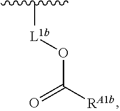

wherein L^{1b} is substituted or unsubstituted alkylene or substituted or unsubstituted heteroalkylene, and R^{A1b} is substituted or unsubstituted, C₄₋₃₀ alkyl, substituted or unsubstituted, C₄₋₃₀ alkenyl, or substituted or unsubstituted, C₄₋₃₀ alkynyl;

L^{2a} is substituted or unsubstituted alkylene or substituted or unsubstituted heteroalkylene;

R^{A2a} is substituted or unsubstituted, C₄₋₃₀ alkyl, substituted or unsubstituted, C₄₋₃₀ alkenyl, or substituted or unsubstituted, C₄₋₃₀ alkynyl; and

R^{B2} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group, or a moiety of the formula:

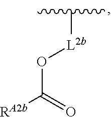

wherein L^{2b} is substituted or unsubstituted alkylene or substituted or unsubstituted heteroalkylene, and R^{A2b} is substituted or unsubstituted, C₄₋₃₀ alkyl, substituted or unsubstituted, C₄₋₃₀ alkenyl, or substituted or unsubstituted, C₄₋₃₀ alkynyl; or
R^{B1} and R^{B2} are joined to form a substituted or unsubstituted, heterocyclic ring or a substituted or unsubstituted, heteroaryl ring.

In certain embodiments, a composition described herein further comprises an amino acid-, peptide- or polypeptide-lipid as described in WO 2013/063468. In certain embodiments, the amine-containing lipidoid is of Formula (X):

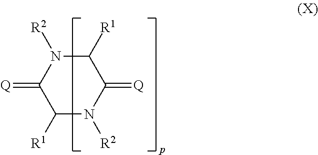

or a pharmaceutically acceptable salt, wherein:

p is an integer of between 1 and 9, inclusive;

each instance of Q is independently O, S, or NR^{Q}, wherein R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), (iii);

each instance of R¹ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —OR^{A1}, —N(R^{A1})₂, —SR^{A1}; wherein each occurrence of R^{A1} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to an sulfur atom, a nitrogen protecting group when attached to a nitrogen atom, or two R^{A1} groups are joined to form an optionally substituted heterocyclic or optionally substituted heteroaryl ring;

or at least one instance of R¹ is a group of formula:

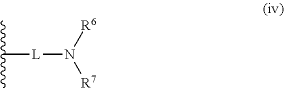

wherein L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, and

R⁶ and R⁷ are each independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group,

each instance of R² is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii); and

Formulae (i), (ii), and (iii) are:

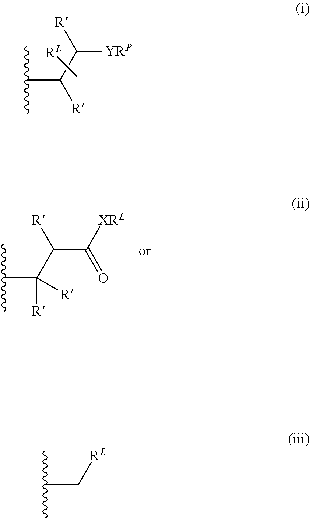

wherein:

each instance of R′ is independently hydrogen or optionally substituted alkyl;

X is O, S, NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;

Y is O, S, NR^{Y}, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;

R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and

R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer;

provided that at least one instance of R^{Q}, R², R⁶, or R⁷ is a group of the formula (i), (ii), or (iii).

In certain particular embodiments, the amino acid-, peptide- or polypeptide-lipid has the formula:

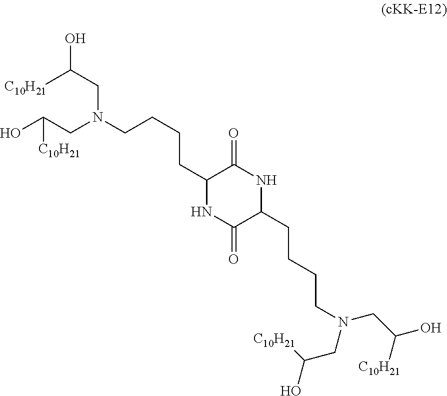

In certain particular embodiments, a composition as described herein can be formulated with C12-200 containing lipid nanoparticles. In some embodiments, the C12-200 is present in a molar percentage of about 1.0% to about 60.0%, about 10.0% to 40.0%, or about 20.0% to about 50.0% of the total composition. In some embodiments, the composition comprises C12-200 in a concentration of about 5.0%, about 7.5%, about 10.0%, about 12.5%, about 15.0%, about 17.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 30.5%, about 31.0%, about 31.5%, about 32.0%, about 32.5%, about 33.0%, about 33.5%, about 34.0%, about 34.5%, about 35.0%, about 35.5%, about 36.0%, about 36.5%, about 37.0%, about 37.5%, about 38.0%, about 38.5%, about 39.0%, about 39.5%, about 40.0%, about 40.5%, about 41.0%, about 41.5%, about 42.0%, about 42.5%, about 43.0%, about 43.5%, about 44.0%, about 44.5%, about 45.0%, about 45.5%, about 46.0%, about 46.5%, about 47.0%, about 47.5%, about 48.0%, about 48.5%, about 49.0%, about 49.5%, about 50.0%, about 50.5%, about 51.0%, about 52.0%, about 53.0%, about 54.0%, about 55.0%, about 56.0%, about 57.0%, about 58.0%, about 59.0% or about 60.0% by mole of the total composition. In certain embodiments, the composition comprises about 50.0% by mole C12-200.

In some embodiments, the lipid nanoparticles can also include one or more auxiliary lipids (also referred to herein as “co-lipids”) including, but not limited to, neutral lipids, amphipathic lipids, PEG-containing lipids, anionic lipids, and sterols.

In some embodiments, the lipid nanoparticles further comprise one or more neutral lipids. Neutral lipids, when present, can be any of a number of lipid species, which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. In some embodiments, the neutral lipid component is a lipid having two acyl groups (e.g., diacylphosphatidylcholine and diacylphosphatidylethanolamine). In some embodiments, the neutral lipid comprises saturated fatty acids with carbon chain lengths in the range of C₁₀ to C₂₀, inclusive, In some embodiments, the neutral lipid includes mono- or di-unsaturated fatty acids with carbon chain lengths in the range of C₁₀ to C₂₀, inclusive. Suitable neutral lipids include, but are not limited to, DPPC (Dipalmitoyl phosphatidylcholine), POPC (Palmitoyl-Oleoyl Phosphatidyl Cholin), DOPE (1,2-dioleoyl-sn-glycero-3-phosphoethanolamine), DSPC (disteroylphosphatidyl choline), egg L-alpha-phosphatidylcholine (EPC); 1,2-dipalmitoyl-sn-glycero-3-phosphoethanolamine (DPPE); and SM (Sphingomyelin). In some embodiments, the neutral lipid is DSPC (disteroylphosphatidyl choline). In some embodiments, the composition comprises DSPC at about 1.0% to about 20.0%, or from about 5.0% to about 10.0% by mole of the total composition. In some embodiments, the composition comprises DSPC at about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0% about 19.5% or about 20.0% by mole of the total composition. In some embodiments, composition comprises about 10% DSPC by mole.

In some embodiments, the lipid nanoparticles further comprise one or more anionic lipids. Anionic lipids are lipids that carry a net negative charge at physiological pH. Anionic lipids, when used in combination with cationic lipids, can reduce the overall surface charge of lipid particles, and/or introduce pH-dependent disruption of lipid structures, facilitating the release of therapeutic agents formulated in the lipid particles (e.g., siRNA molecules). Anionic lipids can include, but are not limited to, fatty acids (e.g., oleic, linoleic, linolenic acids); cholesteryl hemisuccinate (CHEMS); 1,2-di-0-tetradecyl-sn-glycero-3-phospho-(1′-rac-glycerol) (Diether PG); 1,2-dimyristoyl-sn-glycero-3-phospho-(1′-rac-glycerol) (sodium salt); 1,2-dimyristoyl-sn-glycero-3-phospho-L-serine (sodium salt); 1-hexadecanoyl,2-(9Z,12Z)-octadecadienoyl-sn-glycero-3-phosphate; 1,2-dioleoyl-sn-glycero-3-[phospho-rac-(1-glycerol)] (DOPG); dioleoylphosphatidic acid (DOPA); 1,2-dioleoyl-sn-glycero-3-phospho-L-serine (DOPS); and derivatives thereof. Other examples of suitable anionic lipids include, but are not limited to: fatty acids, such as oleic, linoleic, and linolenic acids; and cholesteryl hemisuccinate. Such lipids can be used alone or in combination, for a variety of purposes, such as to attach ligands to the liposome surface.

The lipid nanoparticle can also include one or more lipids capable of reducing aggregation. Examples of lipids that reduce aggregation of particles during formulation include PEG lipids (e.g., DMG-PEG (1,2-Dimyristoyl-sn-glycerol, methoxypolyethylene glycol-PEG), DMA-PEG (poly(ethylene glycol)-dimethacrylate-PEG) and DMPE-PEG550 (1,2-dimyristoyl-sn-glycero-3-phosphoethanolamine-N-[methoxy(polyethylene glycol)-550]), PEG), monosialoganglioside Gm1, and polyamide oligomers (PAO), such as those described in U.S. Pat. No. 6,320,017. The lipid nanoparticles can include DMPE-PEG2000 or DMG-PEG which could be substituted with DMPE-PEG2000 in any of the formulations taught herein. Other suitable PEG lipids include, but are not limited to, PEG-modified phosphatidylethanolamine and phosphatidic acid, PEG-ceramide conjugates (e.g., PEG-CerC₁₄ or PEG-CerC₂₀) (such as those described in U.S. Pat. No. 5,820,873), PEG-modified dialkylamines and PEG-modified 1,2-diacyloxypropan-3-amines, PEG-modified diacylglycerols and dialkylglycerols, mPEG (mw2000)-diastearoylphosphatidylethanolamine (PEG-DSPE).

In some embodiments, a lipid capable of reducing aggregation is DMPE-PEG2000 or DMG-PEG (1,2-Dimyristoyl-sn-glycerol, methoxypolyethylene glycol, PEG). In some embodiments, the compositions comprises about 0.1% to about 5.0% DMPE-PEG2000 or DMG-PEG by mole (i.e., about 0.1% to about 5.0% DMPE-PEG2000 or 0.1% to about 5.0% DMG-PEG) or from about 0.5% to 2.0% DMPE-PEG2000 or DMG-PEG by mole. In some embodiments, the composition comprises about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% DMPE-PEG2000 or DMG-PEG by mole in the total composition. In some embodiments, the composition comprises about 1.5% DMPE-PEG2000 or DMG-PEG by mole.

In some embodiments, the lipid nanoparticle further comprises a sterol. In some embodiments, the sterol is cholesterol. In some embodiments, the composition comprises from about 10.0% to about 50.0% cholesterol by mole, or about 15.0% to about 40.0% cholesterol by mole. In some embodiments, the composition comprises about 10.0%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0%, about 19.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 30.5%, about 31.0%, about 31.5%, about 32.00, about 32.5%, about 33.0%, about 33.5%, about 34.0%, about 34.5%, about 35.0%, about 35.5%, about 36.0%, about 36.5%, about 37.0%, about 37.5%, about 38.0%, about 38.5%, about 39.0%, about 39.5% or about 40.0% cholesterol by mole. In some embodiments, the composition comprises about 38.5% cholesterol by mole.

The ratio of PEG in the LNP formulations can be increased or decreased and/or the carbon chain length of the PEG lipid can be modified from C14 to C18 to alter the pharmacokinetics and/or biodistribution of the LNP formulations.

In some embodiments, the lipid nanoparticles described herein further comprise one or more compounds that are capable of enhancing the cellular uptake or cytosolic distribution of the lipid nanoparticle and/or its encapsulated composition (e.g., gene silencing agent, siRNA molecule, peptide, etc.). Compounds that can enhance the cellular uptake can include levodopa, naphazoline hydrochloride, acetohexamide, niclosamide, diprophylline, and isoxicam, or a combination thereof. Compounds that can enhance the cytosolic distribution can include azaguanine-8, isoflupredone acetate, chloroquine, trimethobenzamide, hydrochloride, isoxsuprine hydrochloride, and diphemanil methylsulfate, or a combination thereof.

In some embodiments, the lipid nanoparticles comprise lipid bilayers encapsulating one or more agents encompassed by the present invention, such as siRNA molecules sufficiently complementary to the mRNA transcription product of at least one biomarker described herein. In some embodiments, the lipid nanoparticles are formulated to facilitate an uptake into cells. In some embodiments, the lipid nanoparticles are formulated to facilitate uptake into monocytes, dendritic cells, and/or macrophages.

The lipid nanoparticle can, in some aspects, further comprise additional agents. In some embodiments, the lipid nanoparticle further comprises one or more antioxidants. Without wishing to be bound by any particular theory, the antioxidant can help stabilize the lipid nanoparticle and prevent, decrease, and/or inhibit degradation of the cationic lipids and/or active agents encapsulated in the lipid nanoparticle. In some embodiments, the antioxidant is a hydrophilic antioxidant, a lipophilic antioxidant, a metal chelator, a primary antioxidant, a secondary antioxidant, or salts or mixtures thereof. In some embodiments, the antioxidant comprises EDTA, or a salt thereof. In some embodiments, the lipid nanoparticle further comprises EDTA in combination with one, two, three, four, five, six, seven, eight, or more additional antioxidants (e.g., primary antioxidants, secondary antioxidants, or other metal chelators). Examples of antioxidants include, but are not limited to, hydrophilic antioxidants, lipophilic antioxidants, and mixtures thereof. Non-limiting examples of hydrophilic antioxidants include chelating agents (e.g., metal chelators) such as ethylenediaminetetraacetic acid (EDTA), citrate, ethylene glycol tetraacetic acid (EGTA), 1,2-bis(o-aminophenoxy)ethane-N,N,N′,N′-tetraacetic acid (BAPTA), diethylene triamine pentaacetic acid (DTPA), 2,3-dimercapto-1-propanesulfonic acid (DMPS), dimercaptosuccinic acid (DMSA), cc-lipoic acid, salicylaldehyde isonicotinoyl hydrazone (SIR), hexyl thioethylamine hydrochloride (HTA), desferrioxamine, salts thereof, and mixtures thereof. Additional hydrophilic antioxidants include ascorbic acid, cysteine, glutathione, dihydrolipoic acid, 2-mercaptoethane sulfonic acid, 2-mercaptobenzimidazole sulfonic acid, 6-hydroxy-2,5,7,8-tetramethylchroman-2-carboxylic acid, sodium metabisulfite, salts thereof, and mixtures thereof. Non-limiting examples of lipophilic antioxidants include vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) (e.g., 2-teri-butyl-4-hydroxyanisole and 3-tert-butyl-4-hydroxyanisole); butylhydroxytoluene (BHT); tert-butylhydroquinone (TBHQ); ascorbyl palmitate; rc-propyl gallate; salts thereof; and mixtures thereof.

In some embodiments, the lipid-based particles formulated for delivery of one or more agents (e.g., gene silencing agents, siRNA molecules, peptides) are selected from lipid vectors, liposomes, lipoplexes, lipid nanoparticles, and micelles. In some embodiments, the lipid-based particle is a pH-sensitive nanoparticle. Such pH-sensitive nanoparticles (PNSDS), which are positive-charge-free nanocarriers comprising siRNA chemically cross-linked with multi-armed poly(ethylene glycol) carriers via acid-labile acetal linkers, can be beneficial for the delivery of siRNA molecules (Tang et al., SiRNA Crosslinked Nanoparticles for the Treatment of Inflammation-induced Liver Injury, Advanced Science, 2016, 4(2), e1600228).

In some embodiments, the lipid nanoparticle further comprises one or more C12-200 aminoalcohol lipids. In some embodiments, the lipid nanoparticle comprises from about 40.0% to about 50.0% C12-200 by mole. In some embodiments, the lipid nanoparticle comprises from about 5.0% to about 10.0% DSPC by mole. In some embodiments, the lipid nanoparticle comprises from about 1.0% to about 2.0% DMG-PEG by mole. In some embodiments, the lipid nanoparticle comprises from about 20.0% to about 40.0% cholesterol by mole. In some embodiments, the lipid nanoparticle comprises 50% C12-200, 10.0% DSPC, 1.5% DMG-PEG, and 38.5% cholesterol by mole.

In some embodiments, the total siRNA molecule moles with respect to the total lipid moles within the formulation ranges from about 1:5 to about 1:20. In some embodiments, the total siRNA molecule moles with respect to the total lipid moles is about 1:5, about 1:6, about 1:7, about 1:8, about 1:9, about 1:10, about 1:11, about 1:12, about 1:13, about 1:14, about 1:15, about 1:16, about 1:17, about 1:18, about 1:18, about 1:19, or about 1:20. In some embodiments, the total siRNA molecule moles with respect to the total lipid moles is about 1:9.

In some embodiments, the lipid nanoparticle (LNP) is formulated to encapsulate an agent, such as an siRNA, using a spontaneous vesicle formation formulation procedure as previously described in Semple et al. (2010) Nat. Biotechnol. 28172-28176.

In some embodiments, the total concentration of one or more agents encompassed by the present invention, such as siRNA molecules that are sufficiently complementary to the mRNA transcription product of at least one biomarker described herein in the formulation is about 0.001 mg/ml to about 100 mg/ml, about 0.01 mg/ml to about 10 mg/ml, or about 0.1 mg/ml to about 20 mg/ml. In some embodiments, the total concentration of two or more, three or more, four or more, five or more, or all six siRNA molecules is about 0.001 mg/ml to about 100 mg/ml, about 0.01 mg/ml to about 10 mg/ml, or about 0.1 mg/ml to about 20 mg/ml.

In some embodiments, the lipid nanoparticles (LNPs) ranging in size from about 40 to about 200 nm, or from about 50 nm to about 100 nm. In some embodiments, the lipid nanoparticle is about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 110 nm, about 120 nm, about 130 nm, about 140 nm, about 150 nm, about 160 nm, about 170 nm, about 180 nm, or about 200 nm in size. In some embodiments, the lipid nanoparticle is about 80 nm in size.

In accordance with the present invention, the formulations as described herein are stable. The term “stable,” as used herein, means remaining in a state or condition that is suitable for administration to a patient. In some embodiments, the formulations are substantially pure. As used herein, “substantially pure” means that the active ingredient (e.g., the siRNA molecules sufficiently complementary to the mRNA transcription product of at least one biomarker described herein) is the predominant species present in the formulation. In some embodiments, a substantially pure composition comprises a composition that is more than 80% comprised of macromolecular species (e.g., active agents, gene silencing agents, siRNA molecules, additional agents (e.g., antioxidants)). In some embodiments, the substantially pure composition comprises a composition that is more than 85%, 90%0, 95%, 96%, 97%, 98%, or 99% comprised of macromolecular species. In some embodiments, the one or more active agents are purified to essential homogeneity (i.e., contaminant species cannot be detected in the composition by conventional detection methods), wherein the composition consists essentially of a single macromolecular species.

Other nanoparticles can be used as delivery vehicles of the agents and compositions described herein. In some embodiments, the nanoparticles comprises chemically and/or enzymatically modified lipoproteins (e.g., apolipoproteins as described in U.S. patent publication No. 2011/0256224). In some embodiments, the nanoparticles comprise other lipoprotein-based nanoparticles, such as HDL, HDL-like lipoprotein particles, or synthetic HDL-like particles (See, e.g., U.S. patent publication No. 2009/0110739; and U.S. Pat. No. 7,824,709).

In some embodiments, nanoparticles with increased macrophage targeted delivery are used to encapsulate a composition as described herein. In some embodiments, the nanoparticle is a GP nanoparticle comprising 1,3-D-glucan (Soto et al. (2012) J. Drug. Deliv. e143524), or a mannosylated chitosan (MCS) nanoparticle (Peng el al. (2015) J. Nanosci. Nanotechnol. 15:2619-2627).

The nanoparticle formulations can be a carbohydrate nanoparticle comprising a carbohydrate carrier. As a non-limiting example, the carbohydrate carrier can include, but is not limited to, an anhydride-modified phytoglycogen or glycogen-type material, phtoglycogen octenyl succinate, phytoglycogen beta-dextrin, anhydride-modified phytoglycogen beta-dextrin. (see, e.g., PCT Publ. No. WO 2012/109121).

In some embodiments, lipid nanoparticles can be engineered to alter the surface properties of particles so the lipid nanoparticles can penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles can be removed from the mucosa tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. (2007) Proc. Natl. Acad. Sci. U.S.A. 104:1482-1487; Lai et al. (2009) Adv Drug Deliv Rev. 61:158-171). The transport of nanoparticles can be determined using rates of permeation and/or fluorescent microscopy techniques including, but not limited to, fluorescence recovery after photo bleaching (FRAP) and high resolution multiple particle tracking (MPT). As a non-limiting example, compositions which can penetrate a mucosal barrier can be made as described in U.S. Pat. No. 8,241,670.

Lipid nanoparticle engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material can be biodegradable and/or biocompatible. The polymeric material can additionally be irradiated. As a non-limiting example, the polymeric material can be gamma irradiated (see, e.g., PCT Publ. No. WO 2012/082165). Non-limiting examples of specific polymers include poly(caprolactone) (PCL), ethylene vinyl acetate polymer (EVA), poly(lactic acid) (PLA), poly(L-lactic acid) (PLLA), poly(glycolic acid)(PGA), poly(lactic acid-co-glycolic acid)(PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone. The lipid nanoparticle can be coated or associated with a co-polymer such as, but not limited to, a block co-polymer, and (poly(ethylene glycol))-(poly(propylene oxide))-(poly(ethylene glycol)) triblock copolymer (see, e.g., U.S. Pat. Publ. Numbers 2012/0121718 and 2010/0003337; and U.S. Pat. No. 8,263,665). The co-polymer can be a polymer that is generally regarded as safe (GRAS) and the formation of the lipid nanoparticle can be in such a way that no new chemical entities are created. For example, the lipid nanoparticle can comprise poloxamers coating PLGA nanoparticles without forming new chemical entities which are still able to rapidly penetrate human mucus (Yang et al. (2011) Angew. Chem. Int. Ed. 50:2597-2600).

For example, LNPs encompassed by the present invention can comprise a PLGA-PEG block copolymer (see, e.g., U.S. Pat. Publ. No. 2012/0004293 and U.S. Pat. No. 8,236,330); a diblock copolymer of PEG and PLA or PEG and PLGA (see, e.g., U.S. Pat. No. 8,246,968); a multiblock copolymer (see, e.g., U.S. Pat. Nos. 8,263,665 and 8,287,910); a polyion complex comprising a non-polymeric micelle and the block copolymer (see, e.g., U.S. Pat. Publ. No. 2012/00768); or amine-containing polymer such as, but not limited to polylysine, polyethylene imine, poly(amidoamine) dendrimers, poly(beta-amino esters) (see, e.g., U.S. Pat. No. 8,287,849).

LNPs encompassed by the present invention can comprise one or more other polymer such as acrylic polymers. Acrylic polymers can include but are not limited to, acrylic acid, methacrylic acid and methacrylic acid copolymersx, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

LNPs encompassed by the present invention can comprise at least one degradable polyester which can contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters can include a PEG conjugation to form a PEGylated polymer. The LNPs can further include at least one targeting ligand. The targeting ligand can be any ligand known in the art such as, but not limited to, a monoclonal antibody (Kirpotin et al. (2006) Cancer Res. 66:6732-6740).

In some embodiments, compositions encompassed by the present invention can be formulated as a solid lipid nanoparticle. A solid lipid nanoparticle (SLN) can be spherical with an average diameter between 10 to 1000 nm. SLN possess a solid lipid core matrix that can solubilize lipophilic molecules and can be stabilized with surfactants and/or emulsifiers. In a further embodiment, the lipid nanoparticle can be a self-assembly lipid-polymer nanoparticle (see, e.g., Zhang el al. (2008) ACS Nano 2:1696-1702).

In some embodiments, agents encompassed by the present invention can be sustained release formulations, such as encapsulated into a nanoparticle or a rapidly eliminated nanoparticle and the nanoparticles or a rapidly eliminated nanoparticle can then be encapsulated into a polymer, hydrogel and/or surgical sealant described herein and/or known in the art. As a non-limiting example, the polymer, hydrogel or surgical sealant can be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.). In another embodiment, the nanoparticle can be encapsulated into any polymer known in the art which can form a gel when injected into a subject. As a non-limiting example, the nanoparticle can be encapsulated into a polymer matrix which can be biodegradable.

In some embodiments, compositions encompassed by the present invention can be formulated as controlled release nanoparticles. In one example, the nanoparticle formulation for controlled release and/or targeted delivery can further include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®). In another example, the controlled release and/or targeted delivery formulation can comprise at least one degradable polyester which can contain polycationic side chains. Degradable polyesters include, but are not limited to, poly(serine ester), poly(L-lactide-co-L-lysine), poly(4-hydroxy-L-proline ester), and combinations thereof.

In another embodiment, the degradable polyesters can include a PEG conjugation to form a PEGylated polymer.

In some embodiments, compositions encompassed by the present invention can be formulated as a lipoplex, such as, without limitation, the ATUPLEX™ system, the DACC system, the DBTC system and other conjugate-lipoplex technology from Silence Therapeutics (London, United Kingdom), STEMFECT™ from STEMGENT® (Cambridge, Mass.), and polyethylenimine (PEI) or protamine-based targeted and non-targeted delivery of therapeutic agents (Aleku et al. (2008) Cancer Res. 68: 9788-9798; Strumberg et al. (2012) Int. J. Clin. Pharmacol. Ther. (2012) 50:76-78; Santel et al. (2006) Gene Ther. 13:1222-1234; Santel et al. (2006) Gene Ther. 13:1360-1370; Gutbier et al. (2010) Pulm. Pharmacol. Ther. 23:334-344; Kaufmann et al. (2010) Microvasc. Res. 80:286-293; Weide et al. (2009) J. Immunother. 32:498-507; Weide et al. (2008) J. Immunother. 31:180-188; Pascolo (2004) Fxp. Opin. Biol. Ther. 4:1285-1294; Fotin-Mleczek et al. (2011) J. Immunother. 34:1-15; Song et al. (2005) Nature Biotechnol. 23:709-717; Peer et al. (2007) Proc. Natl. Acad. Sci. U.S.A. 6:4095-4100; and deFougerolles (2008) Hum. Gene Ther. 19:125-132).

In some embodiments, therapeutic agents and compositions encompassed by the present invention can be encapsulated in, linked to and/or associated with synthetic nanocarriers. Synthetic nanocarriers include, but are not limited to, those described in International Pub. Nos. WO 2010/005740, WO 2010/030763, WO 2012/13501, WO 2012/149252, WO 2012/149255, WO 2012/149259, WO 2012/149265, WO 2012/149268, WO 2012/149282, WO 2012/149301, WO 2012/149393, WO 2012/149405, WO 2012/149411, and WO 2012/149454, and U.S. Pat. Publ. Numbers 2011/0262491, 2010/0104645, 2010/0087337, and 2012/0244222. In another embodiment, the synthetic nanocarrier formulations can be lyophilized, such as by methods described in PCT Publ. No. WO 2011/072218 and U.S. Pat. No. 8,211,473.

In some embodiments, the synthetic nanocarriers can contain reactive groups to release the conjugates described herein (see, e.g., PCT Publ. No. WO 2012/0952552 and U.S. Pat. Publ. No. 2012/0171229). In one embodiment, the synthetic nanocarriers can be formulated for targeted release. In one embodiment, the synthetic nanocarrier is formulated to release the therapeutic agents at a specified pH and/or after a desired time interval. As a non-limiting example, the synthetic nanoparticle can be formulated to release the conjugates after 24 hours and/or at a pH of 4.5 (see, e.g., PCT Publ. Numbers WO 2010/138193 and WO 2010/138194 and U.S. Pat. Publ. Numbers 2011/0020388 and 2011/0027217). In some embodiments, the synthetic nanocarriers can be formulated for controlled and/or sustained release of conjugates described herein. As a non-limiting example, the synthetic nanocarriers for sustained release can be formulated by methods known in the art, described herein and/or as described in PCT Publ. No. WO 2010/138192 and U.S. Pat. Publ. No. 2010/0303850.

In some embodiments, the nanoparticle can be optimized for oral administration. The nanoparticle can comprise at least one cationic biopolymer such as, but not limited to, chitosan or a derivative thereof. As a non-limiting example, the nanoparticle can be formulated by the methods described in U.S. Pat. Publ. No. 20120282343.

In some embodiments, agents encompassed by the present invention can also be formulated using natural and/or synthetic polymers. Non-limiting examples of polymers which can be used for drug delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.). For example, agents and compositions encompassed by the present invention can be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof.

The polymers used in the present invention can have undergone processing to reduce and/or inhibit the attachment of unwanted substances such as, but not limited to, bacteria, to the surface of the polymer. The polymer can be processed by methods known and/or described in the art and/or described in PCT Publ. No. WO 2011/50467.

Nanoparticles can contain one or more polymers. Polymers can contain one more of the following polyesters: homopolymers including glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA,” and caprolactone units, such as poly(ε-caprolactone), collectively referred to herein as “PCL,” and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA,” and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers.” In certain embodiments, the PEG region can be covalently associated with polymer to yield “PEGylated polymers” by a cleavable linker.

The nanoparticles can contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol); polyoxazoline; and copolymers thereof.

The nanoparticles can contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The nanoparticles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both. The particle can contain two or more amphiphilic polymers.

The polymer can also include but is not limited to, polyethenes, polyethylene glycol (PEG), poly(l-lysine) (PLL), PEG grafted to PLL, cationic lipopolymer, biodegradable cationic lipopolymer, polyethylenimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

The polymers can be a cross linkable polyester. Cross linkable polyesters include those known in the art and described in U.S. Pat. Publ. No. 2012/0269761.

The nanoparticles can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose such as methyl cellulose and ethyl cellulose, hydroxyalkyl celluloses such as hydroxypropyl cellulose, hydroxy-propyl methyl cellulose, and hydroxybutyl methyl cellulose, cellulose ethers, cellulose esters, nitro celluloses, cellulose acetate, cellulose propionate, cellulose acetate butyrate, cellulose acetate phthalate, carboxylethyl cellulose, cellulose triacetate, cellulose sulphate sodium salt, polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxyalkanoates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In some embodiments the particle contains biodegradable polyesters or polyanhydrides such as poly(lactic acid), poly(glycolic acid), and poly(lactic-co-glycolic acid).

Degradable polyesters can contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters can include a PEG conjugation to form a PEGylated polymer.

The biodegradable cationic lipopolymer can be made by methods known in the art, such as those described in U.S. Pat. No. 6,696,038 and U.S. Pat Publ. Numbers 2003/0073619 and 2004/0142474. The poly(alkylene imine) can be made using methods known in the art, such as those described in U.S. Pat. Publ. No. 2010/0004315. The biodegradable polymer, biodegradable block copolymer, the biodegradable random copolymer, biodegradable polyester block copolymer, biodegradable polyester polymer, or biodegradable polyester random copolymer can be made using methods known in the art, such as those described in U.S. Pat. Nos. 6,517,869 and 6,267,987. The linear biodegradable copolymer can be made using methods known in the art, such as those described in U.S. Pat. No. 6,652,886. The PAGA polymer can be made using methods known in the art, such as those described in U.S. Pat. No. 6,217,912. The PAGA polymer can be copolymerized to form a copolymer or block copolymer with polymers such as but not limited to, poly-L-lysine, polyarginine, polyornithine, histones, avidin, protamines, polylactides and poly(lactide-co-glycolides). The biodegradable cross-linked cationic multi-block copolymers can be made using methods known in the art, such as those described in U.S. Pat. No. 8,057,821 and U.S. Pat. Publ. No. 2012/009145. For example, the multi-block copolymers can be synthesized using linear polyethylenimine (LPEI) blocks which have distinct patterns as compared to branched polyethyleneimines.

The polymers described herein can be conjugated to a lipid-terminating PEG. As a non-limiting example, PLGA can be conjugated to a lipid-terminating PEG forming PLGA-DSPE-PEG. As another non-limiting example, PEG conjugates for use according to the present invention are described in PCT Publ. No. WO 2008/103276. The polymers can be conjugated using a ligand conjugate such as, but not limited to, conjugates described in U.S. Pat. No. 8,273,363.

Polymer nanoparticles can also comprise chitosan. The chitosan formulation includes a core of positively charged chitosan and an outer portion of negatively charged substrate (see, e.g., U.S. Pat. Publ. No. 2012/0258176). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

Polymer nanoparticles can also comprise PLGA. The PLGA formulations can include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space. In other examples, PLGA microspheres can be formulated by preparing the PLGA microspheres with tunable release rates (e.g., days and weeks) and encapsulating the active agents in the PLGA microspheres while maintaining the integrity of the agent during the encapsulation process.

In some embodiments, Evac, which are non-biodegradable, biocompatible polymers used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; and catheters), can be used. Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Other representative examples of polymer nanoparticles useful according to the present invention include the polymeric compound of PEG grafted with PLL as described in U.S. Pat. No. 6,177,274, as well as suspensions in a solution or medium with a cationic polymer, in a dry pharmaceutical composition or in a solution that is capable of being dried as described in U.S. Pat. Publ. Numbers 2009/0042829 and 2009/0042825.

A polyamine derivative can be used to deliver therapeutic agents and compositions encompassed by the present invention or to treat and/or prevent a disease or to be included in an implantable or injectable device (U.S. Pat. Pubi. No. 2010/0260817). As a non-limiting example the agents encompassed by the present invention can be delivered using a polyamide polymer comprising a 1,3-dipolar addition polymer prepared by combining a carbohydrate diazide monomer with a dilkyne unite comprising oligoamines (U.S. Pat. No. 8,236,280).

Other polymers can include acrylic polymers, such as acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof; or amine-containing polymers such as, but not limited to polylysine, polyethylene imine, poly(amidoamine) dendrimers or combinations thereof; or a PEG-charge-conversional polymer (Pitella et al. (2011) Biomat. 32:3106-3114).

Polymer nanoparticle can further comprise a diblock copolymer. In one embodiment, the diblock copolymer can include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In some embodiments, agents encompassed by the present invention can be formulated with a PLGA-PEG block copolymer (see, e.g., U.S. Pat. Publ. No. US 2012/0004293 and U.S. Pat. No. 8,236,330) or PLGA-PEG-PLGA block copolymers (see, e.g., U.S. Pat. No. 6,004,573). As a non-limiting example, the agents encompassed by the present invention can be formulated with a diblock copolymer of PEG and PLA or PEG and PLGA (see, e.g., U.S. Pat. No. 8,246,968).

In some embodiments, polymer nanoparticles can comprise a plurality of polymers such as, but not limited to hydrophilic-hydrophobic polymers (e.g., PEG-PLGA), hydrophobic polymers (e.g., PEG) and/or hydrophilic polymers (see, e.g., PCT Publ. No. WO 2012/0225129).

In some embodiments, polymer nanoparticles can be formulated as therapeutic nanoparticles. Therapeutic nanoparticles can be formulated by methods and polymers described herein and known in the art such as, but not limited to, PCT Publ. Numbers WO 2010/005740, WO 2010/030763, WO 2010/005721, WO 2010/005723, and WO 2012/054923, and U.S. Pat. Publ. Numbers 2011/0262491, 2010/0104645, 2010/0087337, 2010/0068285, 2011/0274759, 2010/0068286, and 2012/0288541, and U.S. Pat. Nos. 8,206,747; 8,293,276; 8,318,208; and 8,318,211. In some embodiments, therapeutic polymer nanoparticles can be identified by the methods described in U.S. Pat. Publ. No. 2012/0140790.

Polymer formulations can also be selectively targeted through expression of different ligands as exemplified by, but not limited by, folate, transferrin, and N-acetylgalactosamine (GalNAc) (Benoit et al. (2011) Biomacromol. 12:2708-2714; Rozema et al. (2007) Proc. Natl. Acad. Sci. U.S.A. 104:12982-12887; Davis (2009) Mol. Pharm. 6:659-668; Davis (2010) Nature 464:1067-1070).

In some embodiments, the polymer formulation encompassed by the present invention can be stabilized by contacting the polymer formulation, which can include a cationic carrier, with a cationic lipopolymer which can be covalently linked to cholesterol and polyethylene glycol groups. The polymer formulation can be contacted with a cationic lipopolymer using the methods described in U.S. Pat. Publ. No. 2009/0042829. The cationic carrier can include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

The conjugates encompassed by the present invention can be formulated in a polyplex of one or more polymers (see, e.g., U.S. Pat. Publ. Numbers 2012/0237565 and 2012/0270927). In one embodiment, the polyplex comprises two or more cationic polymers. The catioinic polymer can comprise a poly(ethylene imine) (PEI), such as linear PEI.

In some embodiments, other forms of nanoparticles can be used.

For example, agents and compositions encompassed by the present invention can be formulated as a nanoparticle using a combination of polymers, lipids, and/or other biodegradable agents, such as, but not limited to, calcium phosphate. Components can be combined in a core-shell, hybrid, and/or layer-by-layer architecture, to allow for fine-tuning of the nanoparticle so that delivery of the composition encompassed by the present invention. Biodegradable calcium phosphate nanoparticles in combination with lipids and/or polymers have been shown to deliver therapeutic agents in vivo. In one embodiment, a lipid coated calcium phosphate nanoparticle, which can also contain a targeting ligand such as anisamide, can be used to deliver the composition encompassed by the present invention (see, e.g., Li et al. (2010)J. Contr. Rel. 142:416-421; Li et al. (2012) J. Contr. Rel. 158:108-114; Yang et al. (2012) Mol. Ther. 20:609-615). This delivery system combines both a targeted nanoparticle and a component to enhance the endosomal escape, calcium phosphate, in order to improve delivery of the agent.

In some embodiments, the particles can be hydrophobic ion-pairing complexes or hydrophobic ioin-pairs formed by one or more conjugates described above and counterions.

In some embodiments, core-shell nanoparticles can be used for pharmaceutical formulations. The use of core-shell nanoparticles has additionally focused on a high-throughput approach to synthesize cationic cross-linked nanogel cores and various shells (Siegwart et al. (2011) Proc. Natl. Acad. Sci. U.S.A. 108:12996-13001). The complexation, delivery, and internalization of the polymeric nanoparticles can be precisely controlled by altering the chemical composition in both the core and shell components of the nanoparticle. For example, the core-shell nanoparticles can efficiently deliver a therapeutic agent to mouse hepatocytes after they covalently attach cholesterol to the nanoparticle. Core-shell nanoparticles for use with the composition encompassed by the present invention are described and can be formed by the methods described in U.S. Pat. No. 8,313,777.

Inorganic nanoparticles exhibit a combination of physical, chemical, optical and electronic properties and provide a highly multifunctional platform to image and diagnose diseases, to selectively deliver therapeutic agents, and to sensitive cells and tissues to treatment regiments. Not wishing to be bound to any theory, enhanced permeability and retention (EPR) effect of inorganic nanoparticle provides a basis for the selective accumulation of many high-molecular-weight drugs. Circulating inorganic nanoparticles preferentially accumulate at tumor sites and in inflamed tissues (Yuan et al. (1995) Cancer Res. 55:3752-3756) and remain lodged due to their low diffusivity (Pluen et al. (2001) Proc. Natl. Acad. Sci. U.S.A. 98:4628-4633. The size of the inorganic nanoparticles can be nm-500 nm, 10 nm-100 nm, or 100 nm-500 nm. The inorganic nanoparticles can comprise metal (gold, iron, silver, copper, nickel, etc.), oxides (ZnO, TiO₂, Al₂O₃, SiO₂, iron oxide, copper oxide, nickel oxide, etc.), or semiconductor (CdS, CdSe, etc.). The inorganic nanoparticles can also be perfluorocarbon or FeCo.

Inorganic nanoparticles have high surface area per unit volume. Therefore, they can be loaded with therapeutic drugs and imaging agents at high densitives. A variety of methods can be used to load therapeutic drugs into/onto the inorganic nanoparticles, including but not limited to, colvalent bonds, electrostatic interactions, entrapment, and encapsulation. In addition to therapeutic agent drug loads, the inorganic nanoparticles can be funcationalized with targeting moieties, such as tumor-targeting ligands, on the surface. Formulating therapeutic agents with inorganic nanoparticles allows imaging, detection and monitoring of the therapeutic agents.

In some embodiments, agents and compositions encompassed by the present invention is hydrophobic and can be form a kinetically stable complex with gold nanoparticles funcationalized with water-soluble zwitterionic ligands (see, e.g., Kim et al. (2009) JACS 131:1360-1361).

Agents and compositions encompassed by the present invention can be formulated with gold nanoshells. As a non-limiting example, the compositions can be delivered with a temperature sensitive system comprising polymers and gold nanoshells and can be released photothermally (see, e.g., Sershen el al. (2000) J. Biomed. Mater. 51:293-298). Irradiation at 1064 nm was absorbed by the nanoshells and converted to heat, which led to the collapse of the hydrogen and release of the drug. Agents can also be encapsulated inside hollow gold nanoshells, such as by covalent bonding between agents and nanoparticles. Covalent attachment to gold nanoparticles can be achieved through a linker, such as a free thiol, amine or carboxylate functional group. In some embodiments, the linkers are located on the surface of the gold nanoparticles. In some embodiments, agents encompassed by the present invention can be modified to comprise the linkers. The linkers can comprise a PEG or oligoethylene glycol moiety with varying length to increase the particles' stability in biological environment and to control the density of the drug loads. PEG or oligoethylene glycol moieties also minimize nonspecific adsorption of undesired biomolecules. PEG or oligoethylene gycol moieties can be branched or linear (see, e.g., Tong el al. (2009) Langnuir 25:12454-12549). Agents encompassed by the present invention can be tethered to an amine-functionalized gold nanoparticles (see, e.g., Lippard el al. (2009) JACS 131:14652-14653). The cytotoxic effects for the Pt(IV)-gold nanoparticle complex are higher than the free Pt(IV) drugs and free cisplatin.

In some embodiments, agents encompassed by the present invention can be formulated with magnetic nanoparticles, such as those made from iron, cobalt, nickel, and oxides thereof, or iron hydroxide nanoparticles. Localized magnetic field gradients can be used to attract magnetic nanoparticles to a chosen site, to hold them until the therapy is complete, and then to remove them (see, e.g., Alexiou et al. (2000) Cancer Res. 60:6641-6648). In some embodiments, agents encompassed by the present invention can be bonded to magnetic nanoparticles with a linker. The linker can be a linker capable of undergoing an intramolecular cyclization to release agents. Any linker and nanoparticles disclosed can be used (see, e.g., PCT Publ. No. WO 2014/124329). Cyclization can be induced by heating the magnetic nanoparticle or by application of an alternating electromagnetic field to the magnetic nanoparticles.

In some embodiments, agents encompassed by the present invention are loaded onto iron oxide nanoparticles. In some embodiments, the agents encompassed by the present invention are formulated with super paramagnetic nanoparticles based on a core consisting of iron oxides (SPION). SPION are coated with inorganic materials (silica, gold, etc.) or organic materials (phospholipids, fatty acids, polysaccharides, peptides or other surfactants and polymers) and can be further functionalized with drugs, proteins or plasmids.

In one embodiment, water-dispersible oleic acid (OA)-poloxamer-coated iron oxide magnetic nanoparticles are used (see, e.g., Jain Mol. Pharm. (2005) 2:194-205) can be used to deliver the agents. Agents can partition into the OA shell surrounding the iron oxide nanoparticles and the poloxamer copolymers (e.g., Pluronics) confer aqueous dispersity to the formulation.

In some embodiments, nanoparticles having a phosphate moiety are used to deliver agents encompassed by the present invention (see, e.g., U.S. Pat. No. 8,828,975). The nanoparticles can comprise gold, iron oxide, titanium dioxide, zinc oxide, tin dioxide, copper, aluminum, cadmium selenide, silicon dioxide, and/or diamond. The nanoparticles can contain a PEG moiety on the surface.

In some embodiments, agents encompassed by the present invention can be formulated with peptides and/or other conjugates in order to increase penetration of cells such as macrophages and other immune cells. In one embodiment, peptides such as, but not limited to, cell penetrating peptides and proteins and peptides that enable intracellular delivery can be used to deliver pharmaceutical formulations. A non-limiting example of a cell-penetrating peptide that can be used with agents encompassed by the present invention include a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al. (2001)Mol. Ther. 3:310-318; Langel, Cell-Penetrating Peptides: Processes and Applications (CRC Press, Boca Raton Fla., 2002); El-Andaloussi et al. (2003) Curr. Pharm. Des. 11:3597-35611; and Deshayes et al. (2005) Cell. Mol. Life Sci. 62:1839-1849).

In some embodiments, agents encompassed by the present invention can further comprise one or more conjugates that enhance delivery of the active agents (e.g., siRNA molecules) to targeted cells (e.g., monocytes, macrophages, and the like). The conjugate can be a ligand that can be incorporated into lipid formulations to specifically target cells of interest. Using a ligand targeting strategy for lipid particle drug delivery has the advantages of potentially increasing target specificity and avoiding the need for cationic lipids to trigger intracellular delivery. The ligand can include peptides, antibodies, proteins, polysaccharides, glycolipids, glycoproteins, and lectins which make use of mononuclear phagocytes characteristic receptor expression and phagocytic innate processes.

In some embodiments, the conjugated ligand can be a cell targeting peptide (CTP) or a cell-penetrating peptide (CPP) which can improve cell-specific targeting and cell uptake. A few example of the peptides include, but are not limited to muramyl tripeptide (MTP), RGD peptide, GGP-peptide that is selectively associated with monocytes (Karathanasis et al. (2009) Ann. Biomed. Figin. 37:1984-1992). The macrophage peptide targeting agent can also include those identified from phage display and sequencing (see, e.g., Liu et al. (2015) Bioconjug. Chem. 26:1811-1817). In some embodiments the ligand can be antibodies and fragments thereof, Exemplary antibodies specific to monocytes and macrophages include anti-VCAM-1 antibodies, anti-CC52 antibodies, anti-CC531 antibodies, anti-CD11c/DEC-205 antibodies. For example, antibodies can be coupled to the surface of liposomes or distally via their Fc-region to liposome-attached PEG.

In some embodiments, the nanoparticles can be mannosylated by incorporating into the lipid particles a lectin such as alkyl mannosides, Mann-C4-Chol, Mann-His-C4-Chol, Man2DOG, 4-aminophenyl-a-D-mannopyranoside, Aminophenyl-α-D-mannopyranoside, and Man3-DPPE. Immune cells, including alveolar macrophages, peritoneal macrophages, monocyte-derived dendritic cells, and Kupffer cells, constitutively express high levels of the mannose receptor (MR). Macrophages and DCs can therefore be targeted via mannosylated lipid nanoparticles.

Other ligands can also include maleylated bovine serum albumin (MBSA), O-steroly amylopectin (O-SAP), and fibronectin (see, e.g., Ahsan et al. (2002). J. Cont. Rel. 79:29-40: Vyas et al. (2004) Intl. J. Pharm. 269:37-49).

### VII. Administration and Dosing

Agents (e.g., compositions and formulations) described herein can contact desired objects (e.g., cells, cell-free binding partners, and the like) and/or be administered to organisms using well-known methods in the art. For example, agents can be delivered into cells via chemical methods, such as cationic liposomes and polymers, or physical methods, such as gene gun, electroporation, particle bombardment, ultrasound utilization, and magnetofection.

Methods of administration to contact macrophages are well-known in the art, particularly because macrophages are generally present across tissue types (see Ries et al. (2014) Cancer Cell 25:846-859; Perry et al. (2018) J. Exp. Med 215:877-893; Novobrantseva et al. (2012)Mol. Ther. Nucl. Acids 1:e4; Majmudar et al. (2013) Circulation 127:2038-2046; Leuschner et al. (2011) Nat. Biotechnol. 29:11) In addition, administration methods can be tailored to target macrophage populations of interest, such as by using local administration of agents to target spatially restricted populations of macrophages (e.g., intratumoral administration to target TAMs) (see Shirota et al. (2012). J. Immunol. 188:1592-1599; Wang et al. (October 2016) Proc. Natl. Acad. Sci. U.S.A. 113:11525-11530). Such differential administration methods can selectively target macrophage populations of interest while reducing or eliminating contact with other macrophage populations (e.g., intratumoral administration to target TAMs selectively from circulating macrophages).

Agents can also be administered in an effective amount by any route that results in therapeutically effective outcomes. The administration routes can include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

Agents are typically formulated in dosage unit form for ease of administration and uniformity of dosage. It will be understood, however, that the total daily usage of the agents encompassed by the present invention can be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific agent employed; the specific composition employed; the age, body weight, general health, sex and diet of the patient; the time of administration, route of administration, and rate of excretion of the specific agent employed; the duration of the treatment; drugs used in combination or coincidental with the specific compound employed; and like factors well-known in the medical arts.

In some embodiments, agents in accordance with the present invention can be administered at dosage levels sufficient to deliver from about 0.0001 mg/kg to about 1000 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, or from about 10 mg/kg to about 100 mg/kg, or from about 100 mg/kg to about 500 mg/kg, of subject body weight per day, one or more times a day, to obtain the desired therapeutic, diagnostic, prophylactic, or imaging effect. The desired dosage can be delivered three times a day, two times a day, once a day, every other day, every third day, every week, every two weeks, every three weeks, or every four weeks, or every two months. In some embodiments, the desired dosage can be delivered using multiple administrations (e.g., two, three, four, five, six, seven, eight, nine, ten, eleven, twelve, thirteen, fourteen, or more administrations). When multiple administrations are employed, split dosing regimens such as those described herein can be used.

In some embodiments, an agent encompassed by the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors can influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment can increase or decrease over the course of a particular treatment. Changes in dosage can result from the results of diagnostic assays.

As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hour period. It can be administered as a single unit dose.

In some embodiments, the dosage forms can be liquid dosage forms. Liquid dosage forms for parenteral administration include, but are not limited to, pharmaceutically acceptable emulsions, microemulsions, solutions, suspensions, syrups, and/or elixirs. In addition to active ingredients, liquid dosage forms can comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions can be mixed with solubilizing agents such as CREMOPHOR®, alcohols, oils, modified oils, glycols, polysorbates, cyclodextrins, polymers, and/or combinations thereof.

In certain embodiments, the dosages forms can be injectable. Injectable preparations, for example, sterile injectable aqueous or oleaginous suspensions can be formulated according to the known art and can include suitable dispersing agents, wetting agents, and/or suspending agents. Sterile injectable preparations can be sterile injectable solutions, suspensions, and/or emulsions in nontoxic parenterally acceptable diluents and/or solvents, for example, a solution in 1,3-butanediol. Among the acceptable vehicles and solvents that can be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids, such as oleic acid, can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, solid dosage forms of tablets, dragees, capsules, pills, and granules can be prepared with coatings and shells such as enteric coatings and other coatings well-known in the pharmaceutical formulating art. They can optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

Cells can be administered at 0.1×10⁶, 0.2×10⁶, 0.3×10⁶, 0.4×10⁶,0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted can be adjusted based on the desired level of engraftment in a given amount of time. Generally, 1-10⁵ to about 1-10⁹ cells/kg of body weight, from about 1×10⁶ to about 1×10⁸ cells/kg of body weight, or about 1×10⁷ cells/kg of body weight, or more cells, as necessary, can be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective.

Cells can be administered in any suitable route as described herein, such as by infusion. Cells can also be administered before, concurrently with, or after, other anti-cancer agents.

Administration can be accomplished using methods generally known in the art. Agents, including cells, can be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration. For example, subjects of interest can be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine of the present invention is injected to the subject intratumorally or subcutaneously. Cells can be administered in one infusion, or through successive infusions over a defined time period sufficient to generate a desired effect. Exemplary methods for transplantation, engrafiment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Heratol. 27:1418-1427).

Two or more cell types can be combined and administered, such as cell-based therapy and adoptive cell transfer of stem cells, cancer vaccines and cell-based therapy, and the like. For example, adoptive cell-based immunotherapies can be combined with the cell-based therapies of the present invention. In some embodiments, the cell-based agents can be used alone or in combination with additional cell-based agents, such as immunotherapies like adoptive T cell therapy (ACT). For example, T cells genetically engineered to recognize CD19 used to treat follicular B cell lymphoma. Immune cells for ACT can be dendritic cells, T cells such as CD8⁺ T cells and CD4⁺ T cells, natural killer (NK) cells, NK T cells, cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like. The ratio of an agent encompassed by the present invention, such as cancer cells, to another agent encompassed by the present invention or other composition can be 1:1 relative to each other (e.g., equal amounts of 2 agents, 3 agents, 4 agents, etc.), but can modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

Engraftment of transplanted cells can be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or can signal the time for tumor harvesting. Any such metrics are variables that can be adjusted according to well-known parameters in order to determine the effect of the variable on a response to anti-cancer immunotherapy. In addition, the transplanted cells can be co-transplanted with other agents, such as cytokines, extracellular matrices, cell culture supports, and the like.

### VII. Kits

The present invention also encompasses kits for detecting and/or modulating biomarkers described herein. A “kit” is any manufacture (e.g. a package or container) comprising at least one reagent, e.g. a probe or small molecule, for specifically detecting and/or affecting the expression of a marker of the present invention. The kit can be promoted, distributed, or sold as a unit for performing the methods of the present invention. The kit can comprise one or more reagents necessary to detect, express, screen, and the like one or more agents useful in the methods of the present invention. For example, combinations of agents useful for detecting biomarkers encompassed by the present invention (e.g., targets listed in Table 1 and/or Table 2) can be provided in a kit to detect the biomarkers and modulation thereof, which is useful for identifying monocyte and/or macrophage inflammatory phenotype, immune response, anti-cancer function, sensitivity to immune checkpoint therapy, and the like. Such combinations can include one or more agents to detect 1, 2, 3, 4, 5, 6, 7, 8, 9, 10, or more biomarkers inclusive, such as up to and including all of the biomarkers encompassed by the present invention.

In some embodiments, the kit can further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit can be provided in individual containers or as mixtures of two or more reagents in a single container. In addition, instructional materials which describe the use of the compositions within the kit can be included. A kit encompassed by the present invention can also include instructional materials disclosing or describing the use of the kit or an antibody of the disclosed invention in a method of the disclosed invention as provided herein. A kit can also include additional components to facilitate the particular application for which the kit is designed. For example, a kit can additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit can additionally include buffers and other reagents recognized for use in a method of the disclosed invention. Non-limiting examples include agents to reduce non-specific binding, such as a carrier protein or a detergent.

Other embodiments encompassed by the present invention are described in the following Examples. The present invention is further illustrated by the following examples which should not be construed as further limiting.

### EXAMPLES

### Example 1: A Primary Monocyte and Macrophage System Recapitulates Biological Properties of In Vivo Monocytes and Macrophages

Human macrophages exist along a differentiation spectrum from pro-inflammatory (M1-like, also referred to herein as Type 1) to pro-tumorigenic/anti-inflammatory (M2-like, also referred to herein as Type 2) (see, e.g., Biswas et al. (2010) Nat. Immunol. 11: 889-896; Mosser and Edwards (2008) Nat. Rev. Immunol. 8:958-969; Mantovani et al. (2009) Hum. Immunol. 70:325-330). Along this spectrum of functionality, macrophages alter their surface marker expression and morphology, in additional to altering multiple other characteristics. Understanding how these markers change along this spectrum in primary human macrophages is important for understanding what cells are present in a given immunological environment, such as within tumors (tumor-associated macrophages) and/or inflamed tissues, and for understanding how these macrophages affect the immune response within these tissues. Certain cell surface markers, including CD163, CD16, and CD206, traditionally have been used to classify macrophage subtypes. In addition to these surface markers, macrophage subtypes display unique morphologies. M1 macrophages display a dendritic cell-like appearance with increased dendrite projections. M2 macrophages display either a more rounded or spindle-like morphology.

For each monocyte/macrophage cell-based experiment described herein, primary human monocytes/macrophages were used, as opposed to using cell lines, in order to recapitulate the biological properties mimicking in vivo existing cells in the closest possible way that any in vitro experimental system with isolated cell types allows. In particular, the system provides access to studying natural biological properties of primary cells and provides access to natural diversity arising from different donors having different genetic and environmental exposures. Therefore, it is important to consider natural genetic and immunological variability among the human population when interpreting the results of the assays.

Monocytes were differentiated in vitro to M1-like (Type 1) and/or M2-like (Type 2) phenotypes (Ries et a. (2014) Cancer Cell 25:846-859; Vogel et al. (2014) Immunobiol. 219:695-703). In order to differentiate monocytes into M1 versus M2 phenotypes monocytes were isolated from whole blood of healthy donors by Ficoll separation with RosetteSep™ Human Monocyte Enrichment Cocktail (Stemcell Technologies, Vancouver, Canada) according to the manufacturer's instructions. Isolated monocytes were arrayed in 24-well plates overnight in IMDM Media containing 10% fetal bovine serum and non-adherent cells were washed off after 24 hours. Monocytes were differentiated into macrophages by culturing for 6 days in IMDM 10% FBS plus 50 ng/ml human M-CSF for M2 macrophages, or 50 ng/ml GM-CSF (Biolegend, San Diego, Calif.) for M1 macrophages. After 6 days, M1 macrophages were activated with 10 ng/ml human interferon gamma and 100 ng/ml LPS (Invivogen, San Diego, Calif.) and M2-like macrophages were divided into two separate cultures where each was further induced into M2c macrophages by adding IL- and M2d macrophages by adding IL-4, IL-10 and TGF-β, respectively. At day 8, macrophages were harvested and processed for further analysis. Expression of M1 and M2 macrophage markers and surface expressed targets was assessed by flow cytometry. For flow cytometry, cells were collected and resuspended in 50 ul FACS buffer (PBS+2.5% FBS+0.5% sodium azide) and blocked for 15 minutes with TruStain FcX™ (Biolegend Cat. No. 422302) on ice. Antibodies (Table 3) were diluted in FACS buffer according to the manufacturer's instructions and added to cells for 15 minutes on ice.

Labeled cells were washed twice with FACS buffer and fixed with PBS+2% paraformaldehyde for flow cytometry analysis on an Attune™ flow cytometer (ThermoFisher). Data were analyzed via FlowJo software. Morphology was assessed via microscopy.

TABLE 3
Flow Antibodies

| Antigen | Clone | Source |
|---|---|---|
| CD163 | 215927 | RnD Systems |
| CD16 | 3a8 | BioLegend |
| CD206 | 15-2 | BioLegend |
| SIGLEC9 | 191240 | RnD Systems |
| LCP2 | 130407-085 | Miltenyi Biatec |
| CLEC7A | 15E2 | BioLegend |
| SIGLEC7 | 194211 | RnD Systems |
| CD33 | WM53 | Biolegend |
| SELPLG | 688101 | RnD Systems |
| CCR5 | J418F1 | BioLegend |
| CD84 | CD84.1.21 | BioLegend |
| C3AR1 | hC3ARZ8 | BioLegend |
| CD37 | MB371 | BD Biosciences |
| VISG4 | JAV4 | eBioscience |
| LILRB2 | 287219 | RnD Systems |
| TIM3 | 344823 | RnD Systems |
| TLR8 | 935166 | RnD Systems |
| CD48 | 394607 | RnD Systems |
| CD53 | H129 | BioLegend |
| SRPa | SE5A5 | BioLegend |
| CD74 | LN2 | BioLegend |
| RP105 | MHR73-11 | BioLegend |
| CD45 | 2D1 | BioLegend |
| CD3 | OKT3 | BioLegend |
| CD4 | A161A1 | BioLegend |
| CD19 | HIB19 | BioLegend |
| PD-1 | NAT105 | BioLegend |
| CD11b | ICRF44 | BioLegend |
| CD8a | RPA-T8 | BioLegend |
| CD14 | M5E2 | BioLegend |
| CD56 | 5.1H11 | BioLegend |
| IFNg | 4S.B3 | BioLegend |
| Granzyme B | GB11 | BioLegend |

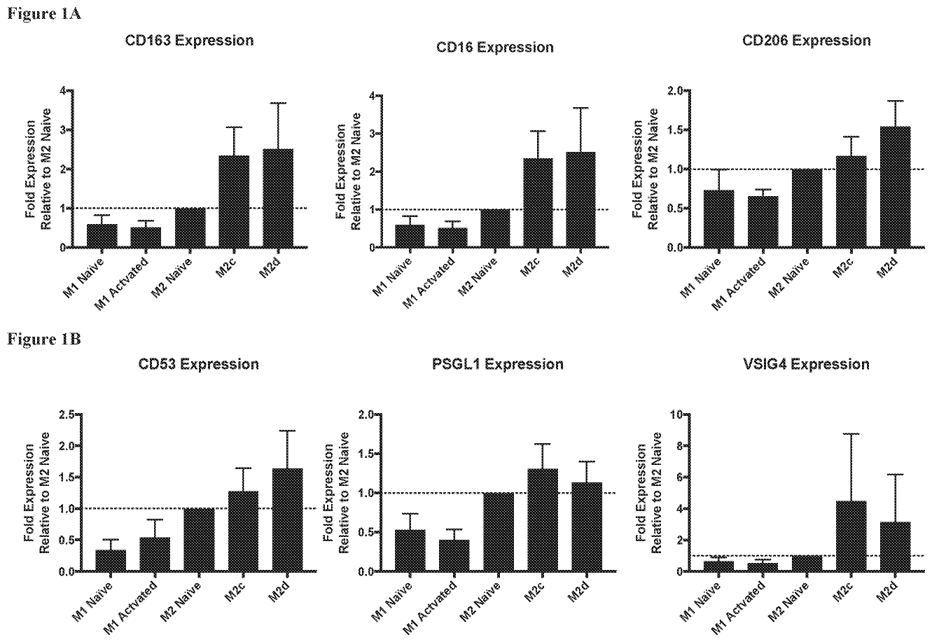

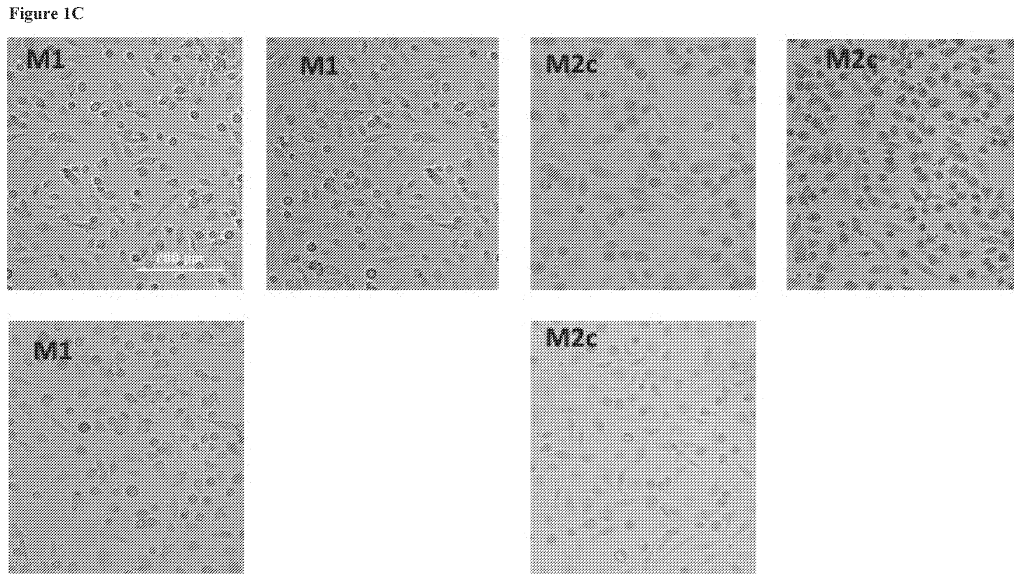

The skewing of macrophages towards an M2 phenotype was shown to upregulate CD163, CD16, and CD206 relative to M1 macrophages (FIG. 1A). In addition to these classic markers, FIG. 1B shows that the new biomarkers described herein, such as CD53, PSGL1, and VSIG4, are also upregulated on M2 macrophages. FIG. 1C demonstrates the morphological differences that exist within the macrophage spectrum and, importantly, demonstrates the variability that exists within primary human cells.

### Example 2: Validation of Targets that Modulate Macrophage Inflammatory Phenotype by Target Nucleic Acid Knockdown

In order to validate the ability of macrophage associated targets described herein to modulate macrophage phenotype, target knockdown experiments were performed, such as by using target-specific siRNAs that were designed, validated, and tested in primary human macrophages.

siRNAs were synthesized by AXO Labs (Kulmbach, Germany). Oligoribonucleotides were synthesized using phosphoramidite technology on solid phase employing an ABI 394 synthesizer (Applied Biosystems) at 10 μmol scale. Syntheses were performed on a solid support made of controlled pore glass (CPG, 520\acute{Å}, with a loading of 75 μmol/g, obtained from Prime Synthesis, Aston, Pa., USA). Regular RNA phosphoramidites, 2′-O-methylphosphoramidites, and ancillary reagents were purchased from Proligo (Hamburg, Germany). Specifically, the following amidites were used: (5′-O-dimethoxytrityl-N6-(benzoyl)-2′-O-t-butyldimethylsilyl-adenosine-3′-O-(2-cyanoethyl-N,N-diisopropylamino) phosphoramidite, 5′-O-dimethoxytrityl-N4-(acetyl)-2′-O-t-butyldimethylsilyl-cytidine-3′-O-(2-cyanoethyl-N,N-diisopropylamino) phosphoramidite, (5′-O-dimethoxytrityl-N2-(isobutyryl)-2′-O-t-butyldimethylsilyl-guanosine-3′-O-(2-cyanoethyl-N,N-diisopropylamino) phosphoramidite, and 5′-O-dimethoxytrityl-2′-O-t-butyldimethylsilyl-uridine-3′-O-(2-cyanoethyl-N,N-diisopropylamino) phosphoramidite. 2′-O-Methylphosphoramidites carried the same protecting groups as the regular RNA amidites. All amidites were dissolved in anhydrous acetonitrile (100 mM) and molecular sieves (3\acute{Å}) were added. 5-ethyl thiotetrazole (ETT, 500 mM in acetonitrile) was used as activator solution. Coupling times were 6 minutes. In order to introduce phosphorothioate linkages, a 50 mM solution of 3-((N,N-dimethylaminomethylidene)amino)-3H-1,2,4-dithiazole-5-thione (DDTT, obtained from Chemgenes, Wilmington, Mass., USA) in anhydrous acetonitrile was employed.

After finalization of the solid phase synthesis, the dried solid support was transferred to a 15 mL tube and treated with methylamine in methanol (2M, Aldrich) for 180 min at 45° C. After centrifugation, the supernatant was transferred to a new 15 mL tube and the CPG was washed with 1200 μL N-methylpyrolidin-2-one (NMP, Fluka, Buchs, Switzerland). The washing was combined with the methanolic methylamine solution and 450 μL triethylamine trihydrofluoride (TEA·3HF, Alfa Aesar, Karlsruhe, Germany) was added. This mixture was brought to 65° C. for 150 min. After cooling to room temperature, 0.75 mL NMP and 1.5 mL of ethoxytrimethylsilane (Merck, Darmstadt, Germany) was added. Ten minutes later, the precipitated oligoribonucleotide was collected by centrifugation, the supernatant was discarded, and the solid was reconstituted in 1 mL Buffer A (described below).

Crude oligomers were purified by anionic exchange HPLC using a column packed with Source Q15 (GE Healthcare) and an AKTA Explorer system (GE Healthcare). Buffer A was 10 mM sodium perchlorate, 20 mM Tris, 1 mM EDTA, pH 7.4 (Sigma Aldrich) and contained 20% acetonitrile. Buffer B was the same as Buffer A with the exception of 500 mM sodium perchlorate. A gradient of 22% B to 42% B within 42 column volumes (CV) was employed. UV traces at 280 nm were recorded. Appropriate fractions were pooled and precipitated with 3M NaOAc, pH of 5.2, and 70% ethanol. Finally, the pellet was washed with 70% ethanol. Alternatively, desalting was carried out using Sephadex® HiTrap® columns (GE Healthcare) according to the manufacturer's recommendations. The concentration of the solution was determined by absorbance measurement at 260 nm in a UV photometer (Eppendorf, Hamburg, Germany). Until annealing, the individual strands were stored as frozen solutions at −20° C.

Complementary strands were annealed by combining equimolar RNA solutions. The mixture was lyophilized and reconstituted with an appropriate volume of annealing buffer (100 mM NaCl, 20 mM sodium phosphate, pH 6.8) to achieve the desired concentration. This solution was placed into a water bath at 75° C. and was cooled to room temperature within 2 hours.

Dose response curves for siRNAs were generated in vitro using a variety of cell lines according to Table 4.

TABLE 4
Cell lines and conditions for siRNA analyses

| | | Cell Seeding | Endpoint Assay to Measure mRNA |
|---|---|---|---|
| Gene | Cell Line | Density per Well | Knockdown |
| SIGLEC9 | Hepa1-6 + reporter plasmid | 15,000 | Dual-Glo Luciferase Assay |
| CD74 | THP-1 | 25,000 | bDNA Assay |
| SELPLG | THP-1 | 25,000 | bDNA Assay |
| VSIG4 | Hepa1-6 + reporter plasmid | 15,000 | Dual-Glo Luciferase Assay |
| LRRC25 | Hepa1-6 + reporter plasmid | 15,000 | Dual-Glo Luciferase Assay |
| CD84 | Hepa1-6 + reporter plasmid | 15,000 | Dual-Glo Luciferase Assay |
| IGSF6 | Hepa1-6 + reporter plasmid | 15,000 | Dual-Glo Luciferase Assay |
| CD48 | Hepa1-6 + reporter plasmid | 15,000 | Dual-Glo Luciferase Assay |
| CD33 | SK-MEL-2 | 15,000 | bDNA Assay |
| CD207 | Hepal-6 + reporter plasmid | 15,000 | Dual-Glo Luciferase Assay |
| LILRB2 | Hepa1-6 + reporter plasmid | 20,000 | Dual-Glo Luciferase Assay |
| EVI2B | A172 | 15,000 | bDNA Assay |
| CLEC7A | Hepa1-6 + reporter plasmid | 20,000 | Dual-Glo Luciferase Assay |
| SIGLEC7 | Hepa1-6 + reporter plasmid | 20,000 | Dual-Glo Luciferase Assay |
| AIF1 | THP-1 | 25,000 | bDNA Assay |
| LST1 | THP-1 | 25,000 | bDNA Assay |
| TNFAIP8L2 | THP-1 | 25,000 | bDNA Assay |
| SPI1 | THP-1 | 25,000 | bDNA Assay |
| TBXAS1 | A549 | 15,000 | bDNA Assay |
| CD37 | THP-1 | 25,000 | bDNA Assay |
| CD53 | THP-1 | 25,000 | bDNA Assay |
| FERMT3 | THP-1 | 25,000 | bDNA Assay |
| CXorf21 | THP-1 | 25,000 | bDNA Assay |
| CCR5 | Hepal-6 + reporter plasmid | 20,000 | Dual-Glo Luciferase Assay |
| DOCK2 | THP-1 | 25,000 | bDNA Assay |

Briefly, cells were seeded in 96-well plates and reverse transfected with siRNA, Lipofectamine® 2000 (0.5 uL/well; ThermoFisher), and, in some cases, a reporter plasmid (50 ng/well). The reporter plasmid (psiCHECK™-2, Promega) encoded both firefly luciferase and the gene of interest fused to renilla luciferase; silencing of the gene of interest results in proportional silencing of renilla luciferase but leaves firefly luciferase unaffected as an internal control.

After 24 hr incubation at 37° C., mRNA knockdown was measured using an endpoint assay. For targets without a reporter plasmid, a branched DNA (bDNA) assay was performed according to the manufacturer's instructions (QuantiGene® Singleplex Gene Expression Assay, ThermoFisher) to measure both the target gene and GAPDH (housekeeping gene) mRNA. Data are plotted as siRNA concentration (nM) vs. remaining mRNA (ratio of target gene to GAPDH, normalized to mock siRNA-transfected cells). For targets with a reporter plasmid, a Dual-Glo® Luciferase Assay was performed according to the manufacturer's instructions (Promega) to measure firefly and renilla luciferase expression. Data are plotted as siRNA concentration (nM) vs. remaining mRNA (ratio of renilla to firefly luminescence, normalized to mock siRNA-transfected cells).

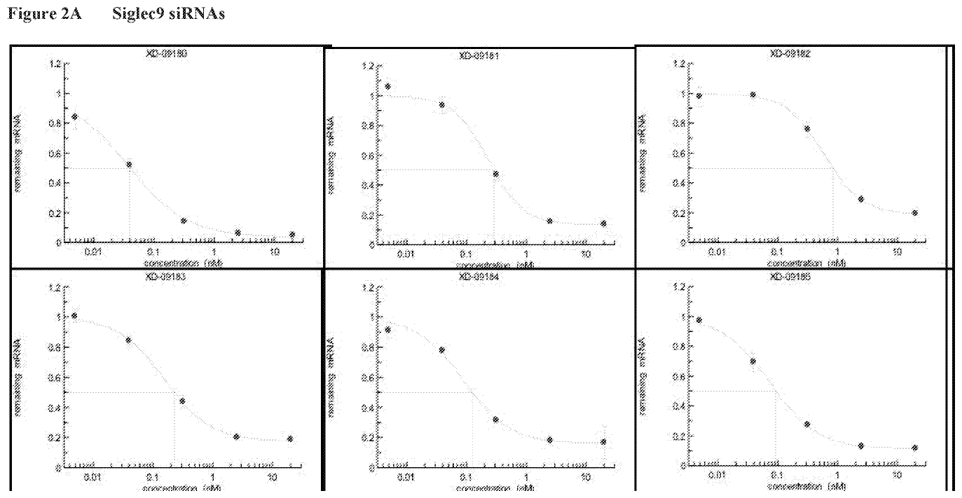

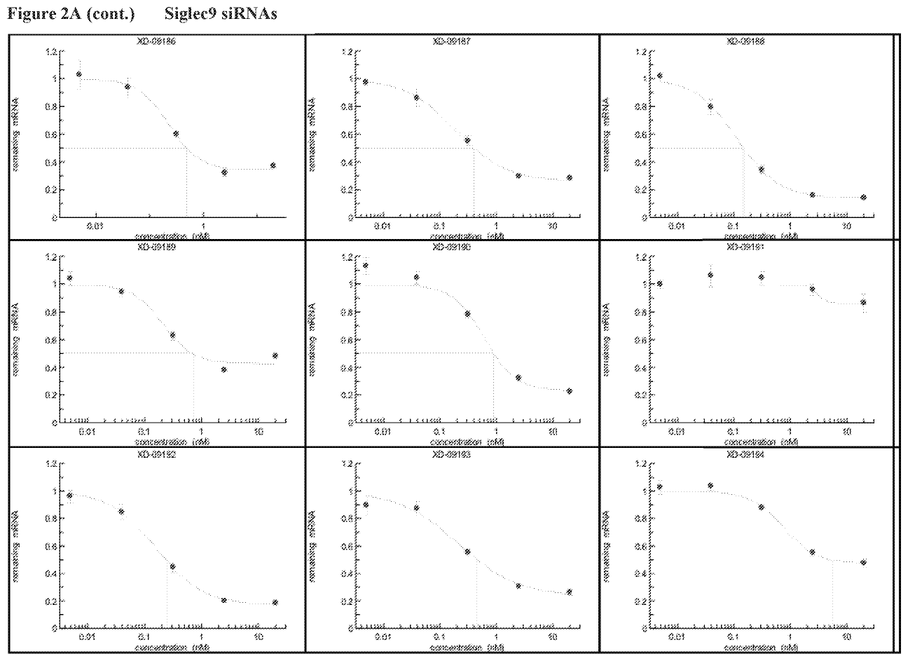

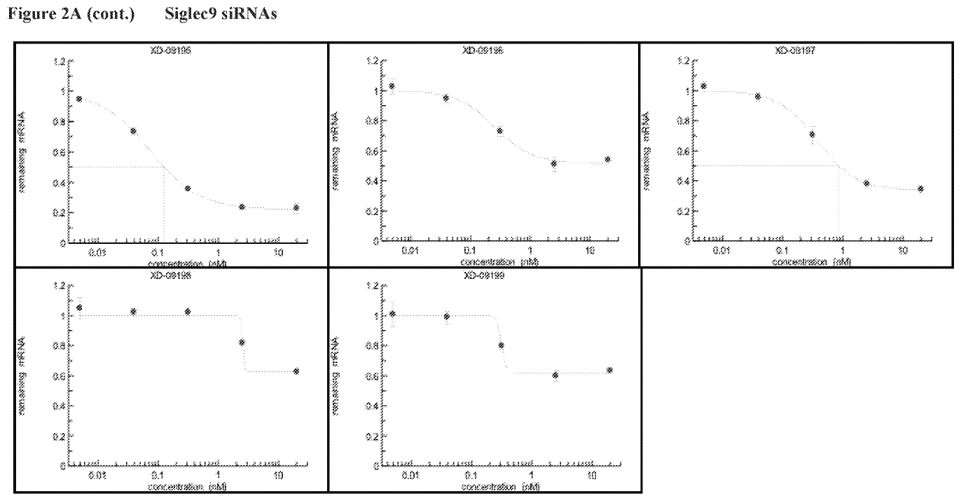

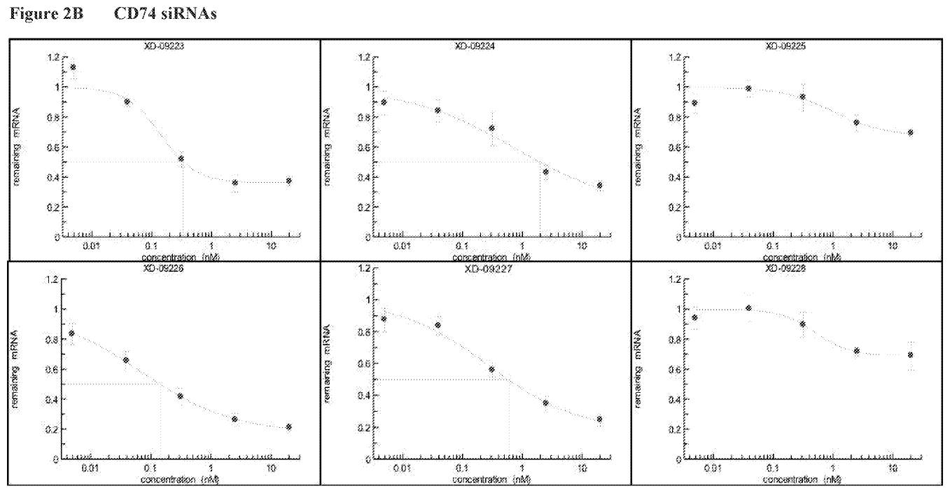

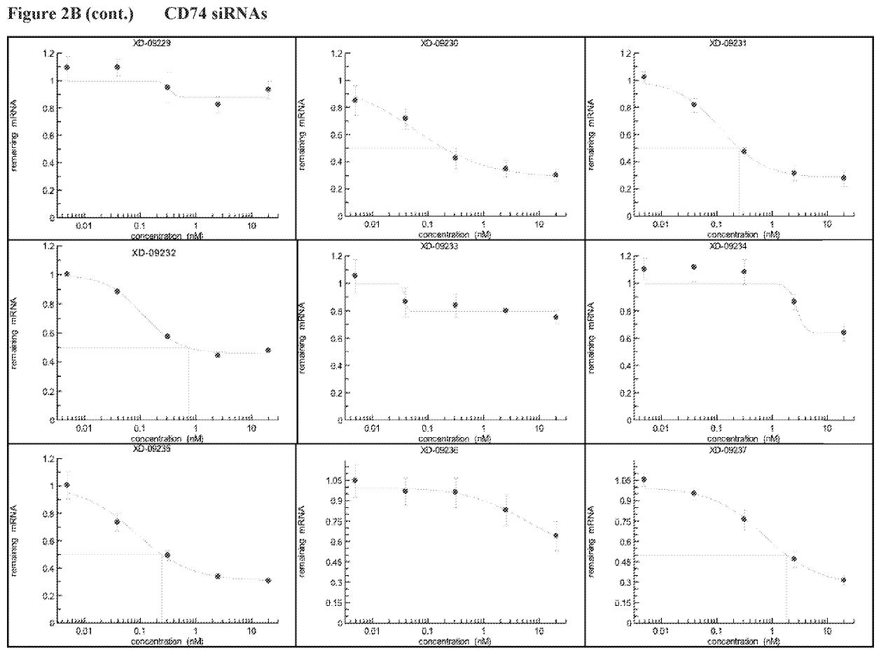

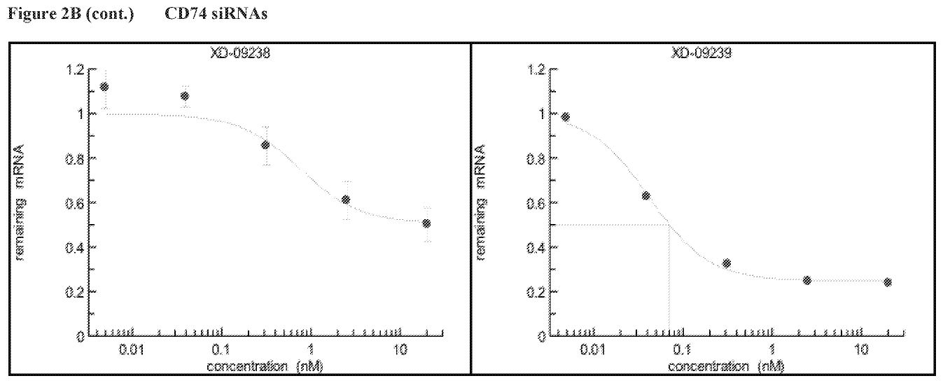

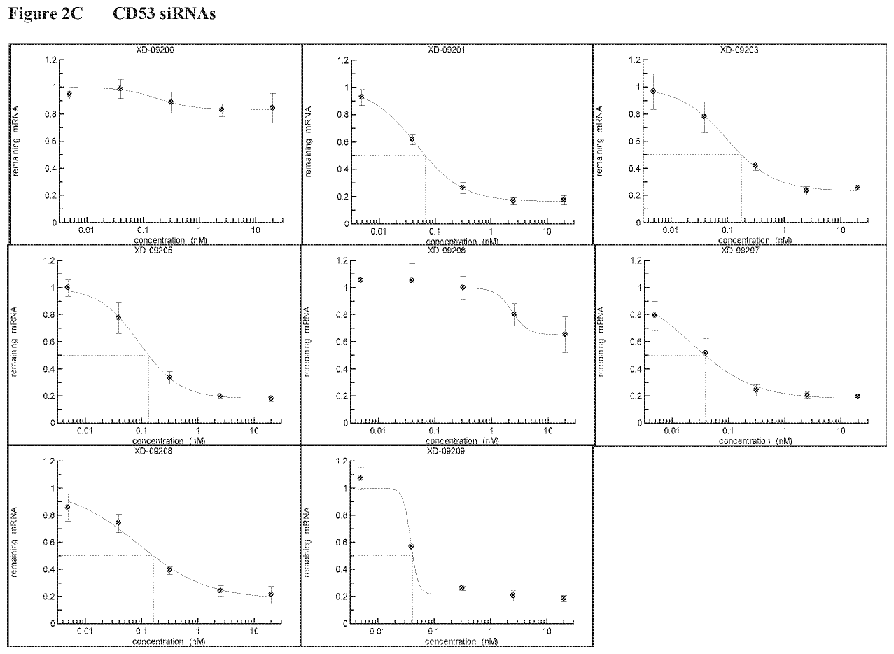

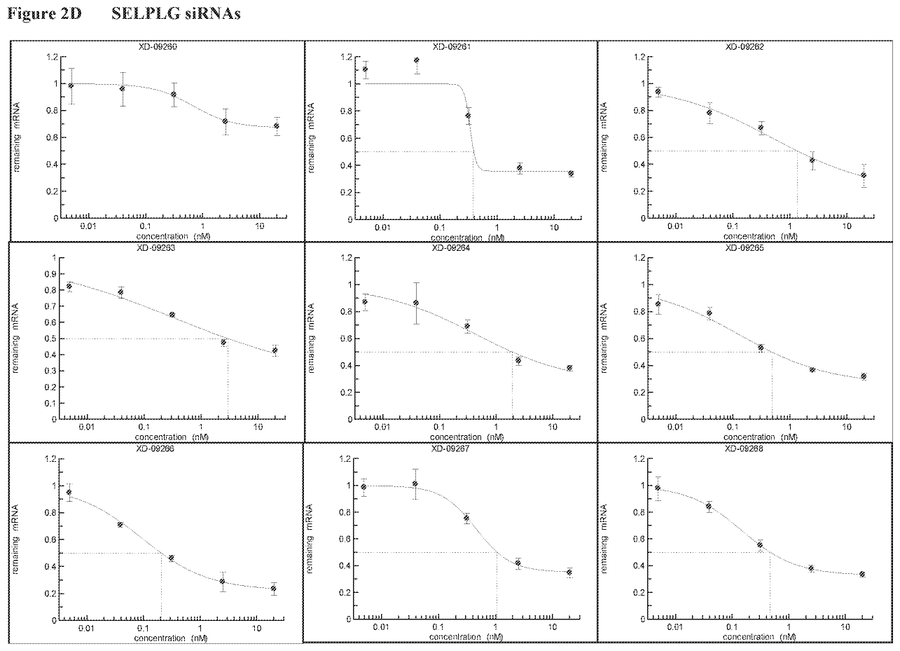

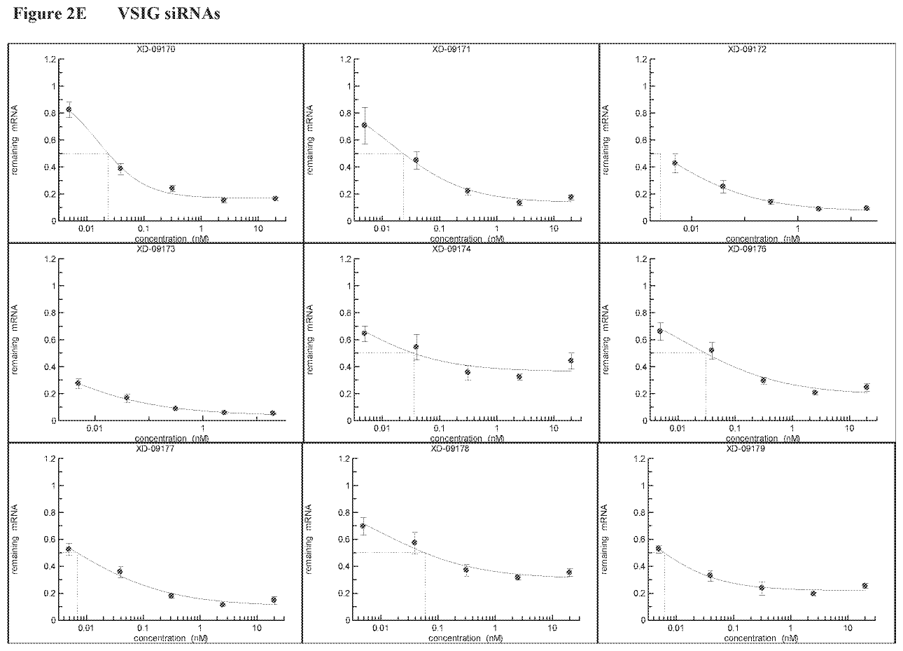

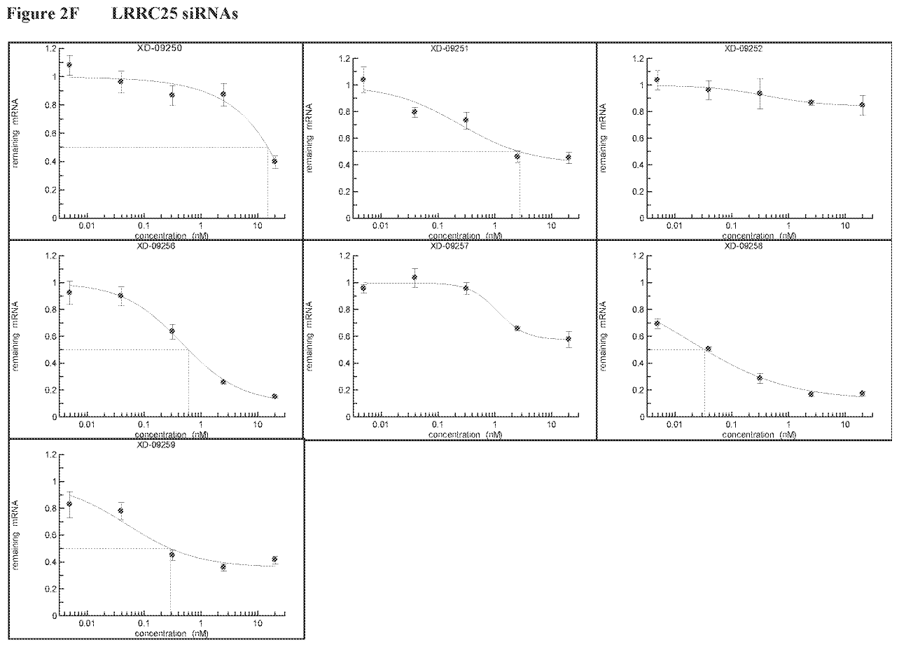

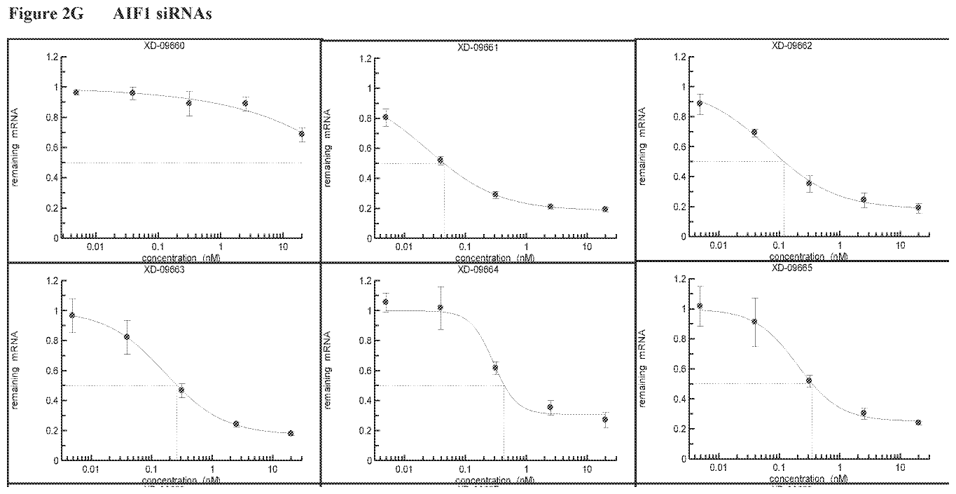

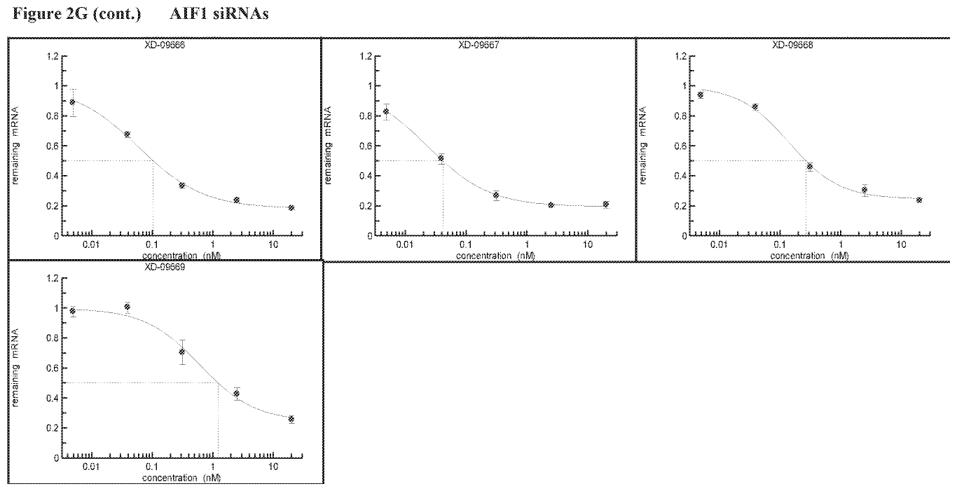

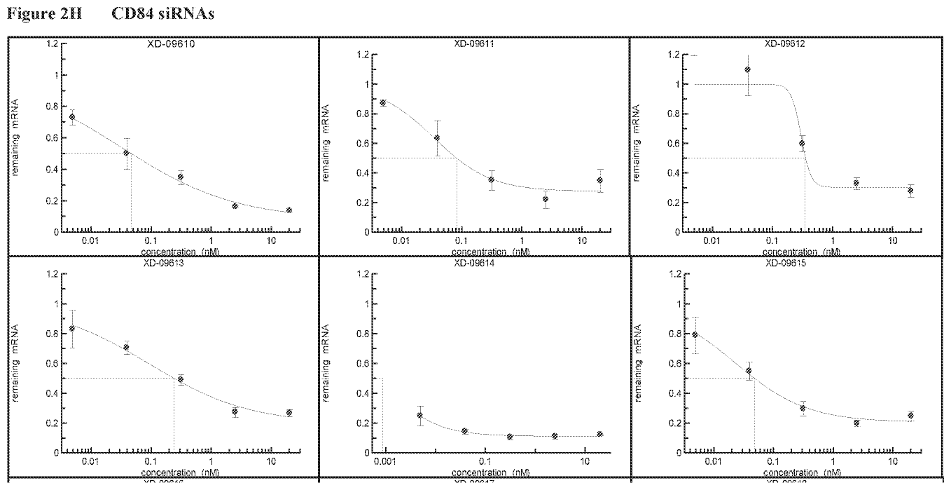

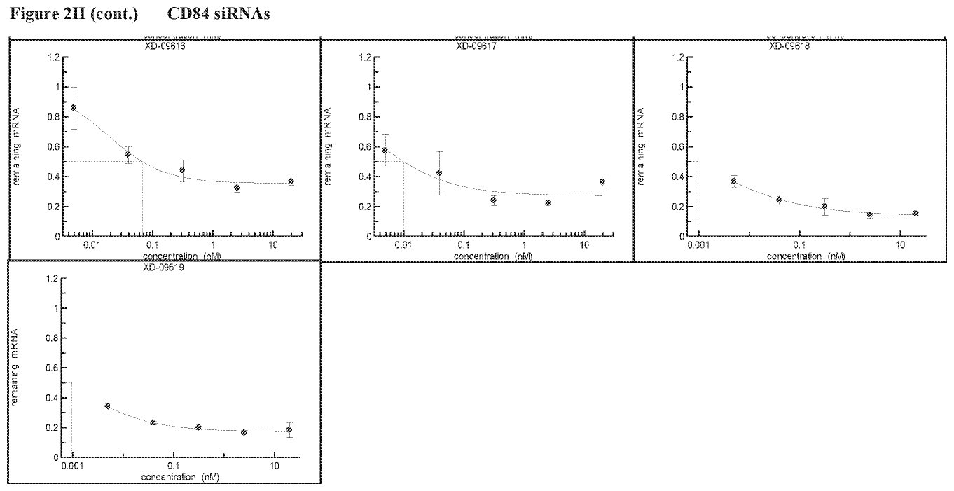

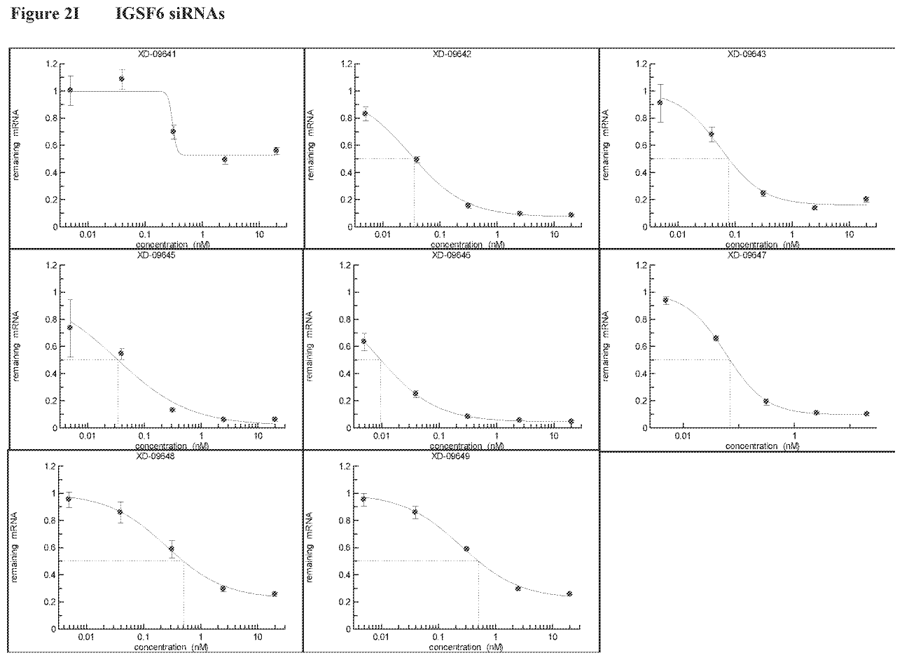

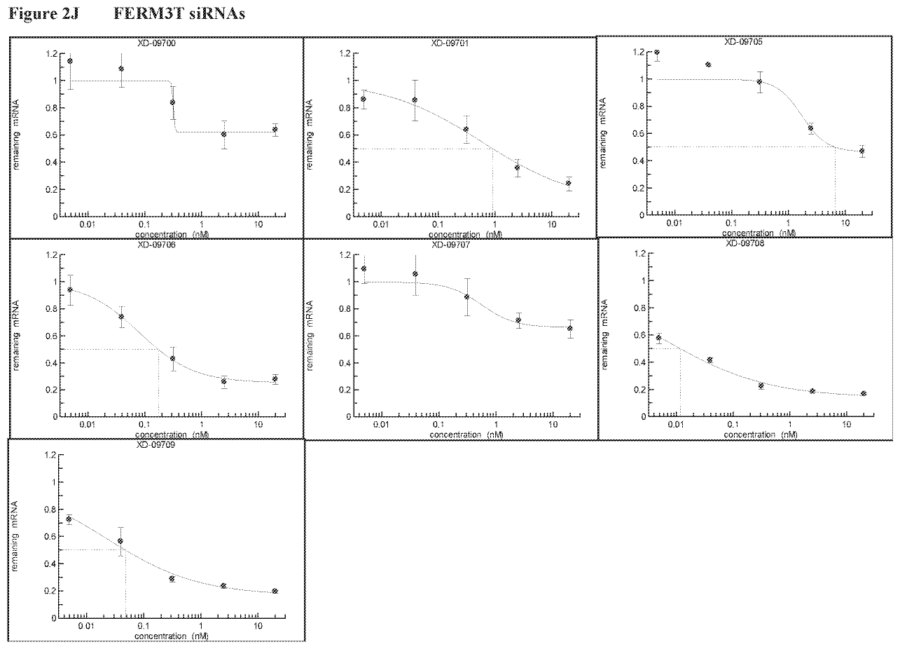

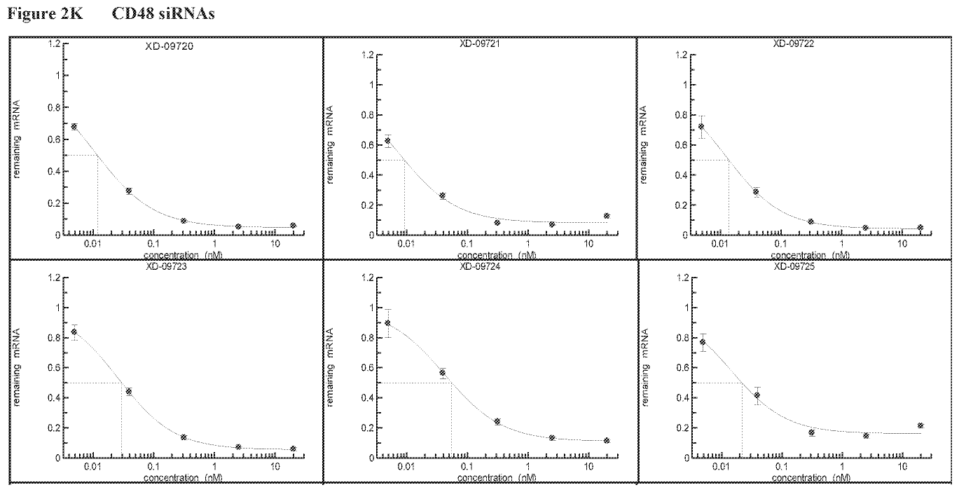

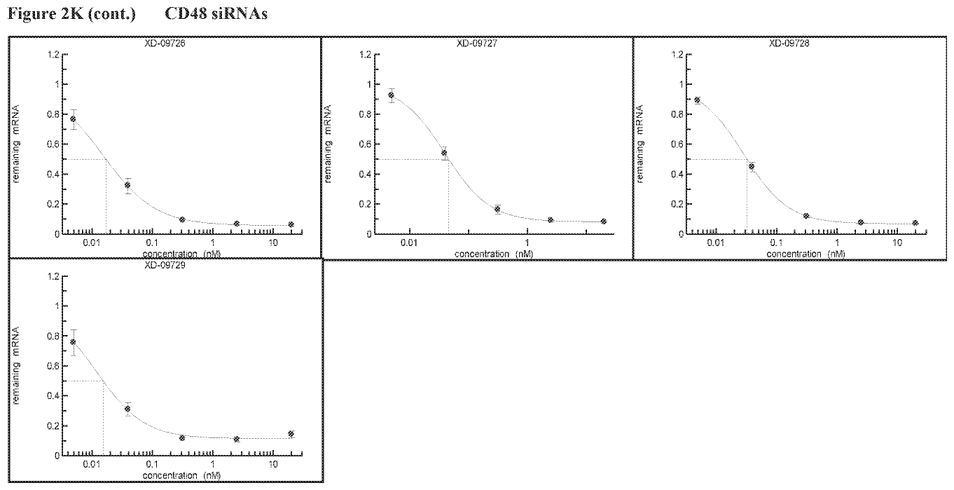

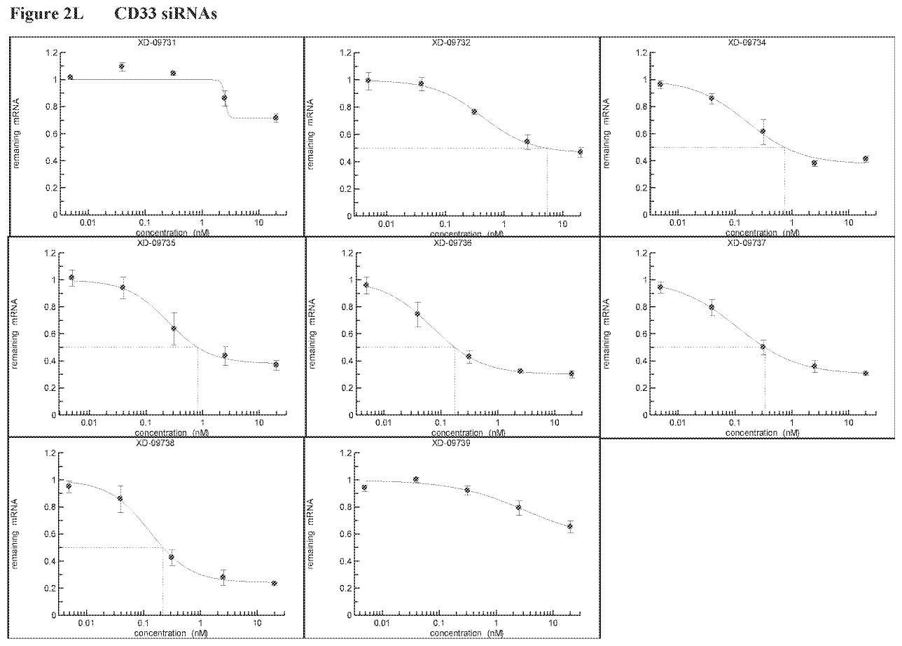

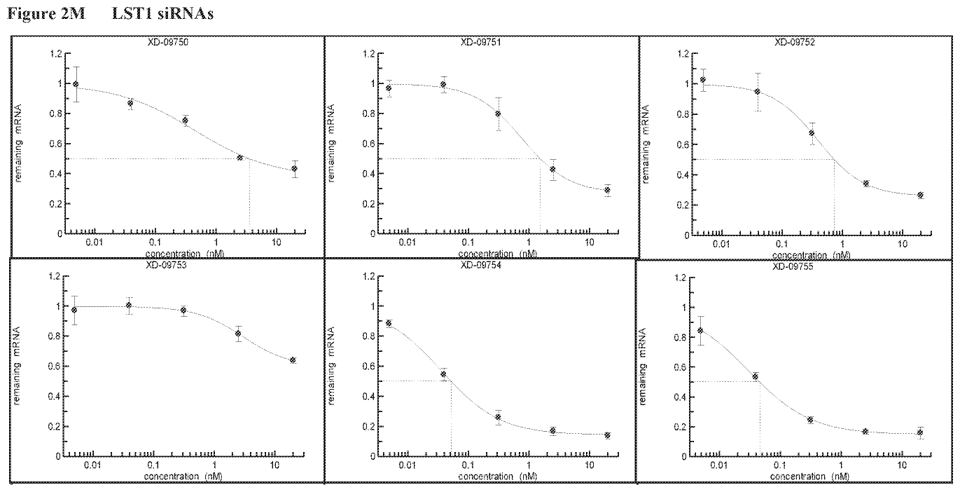

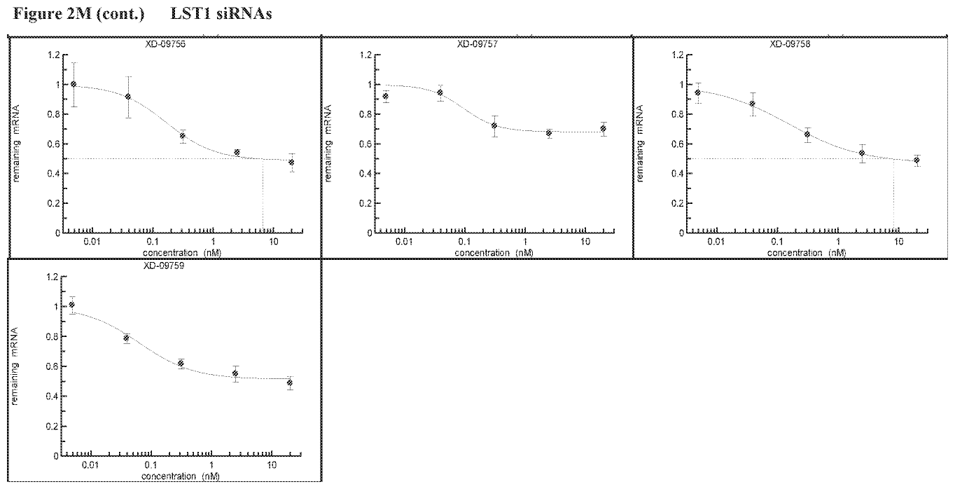

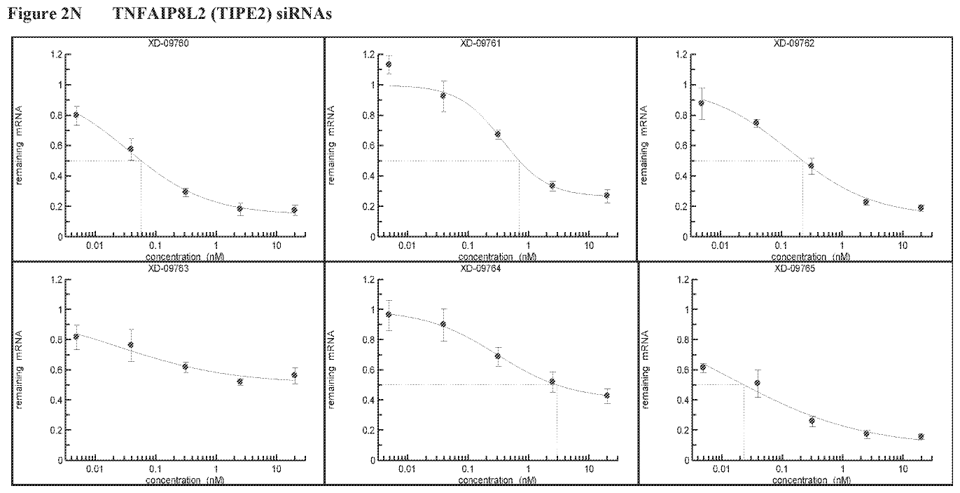

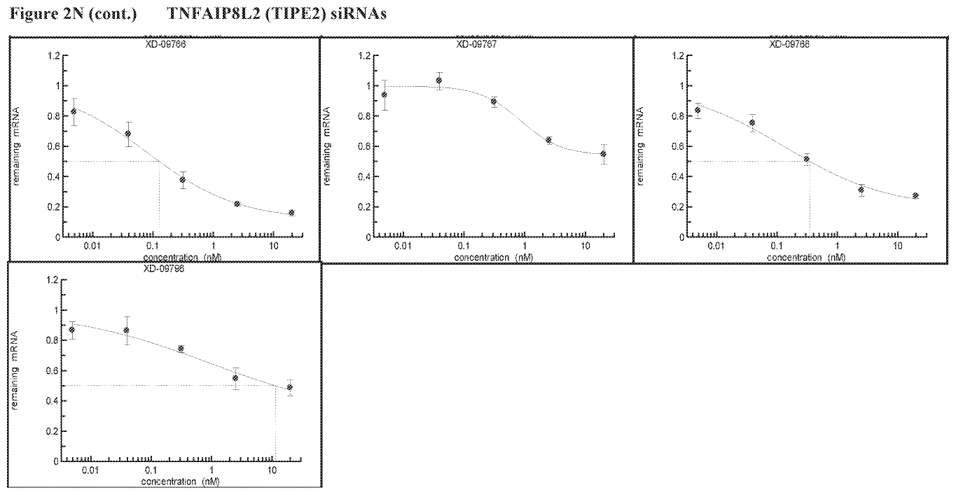

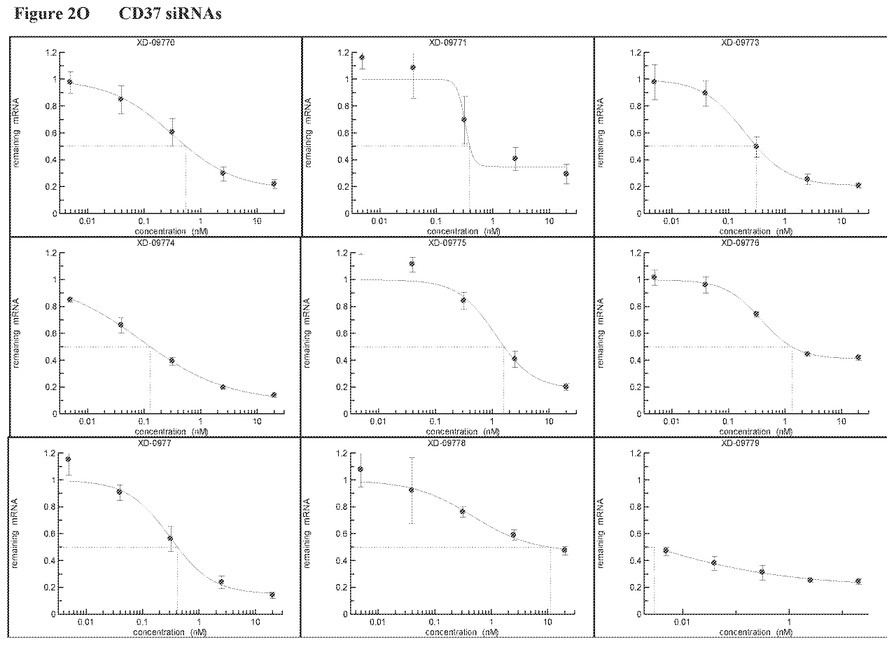

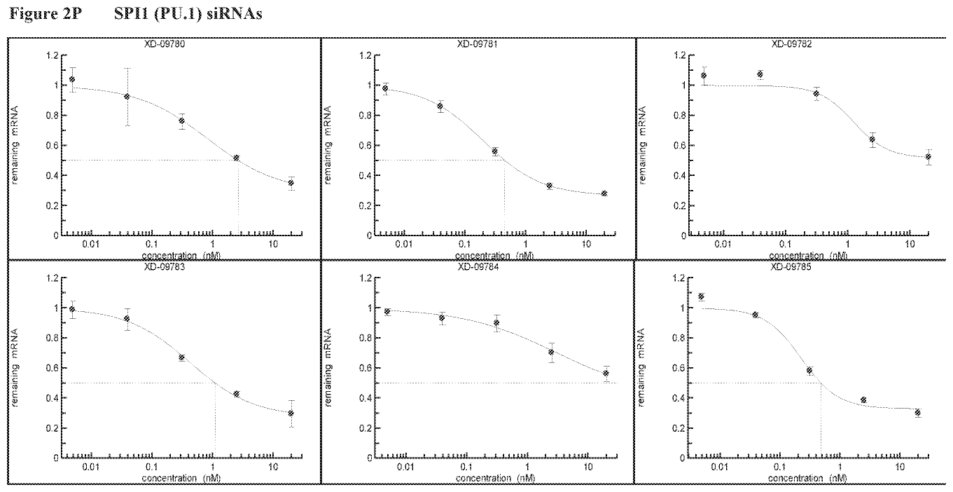

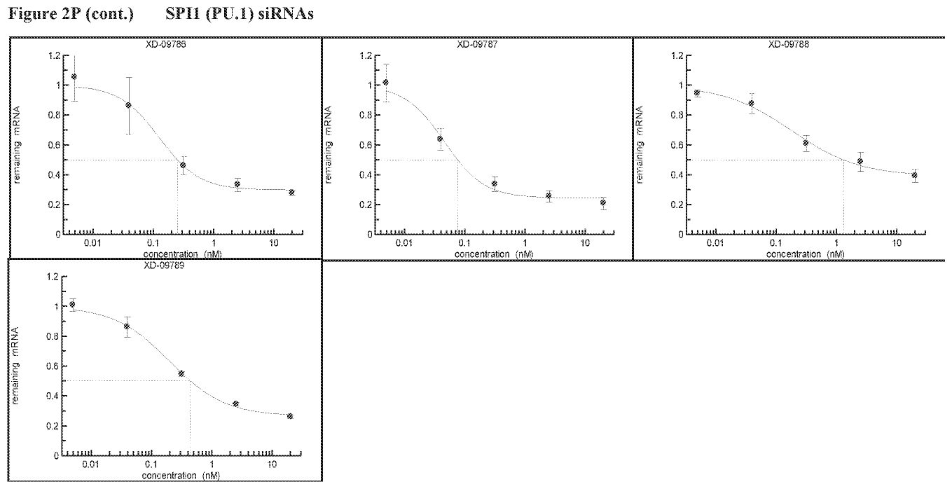

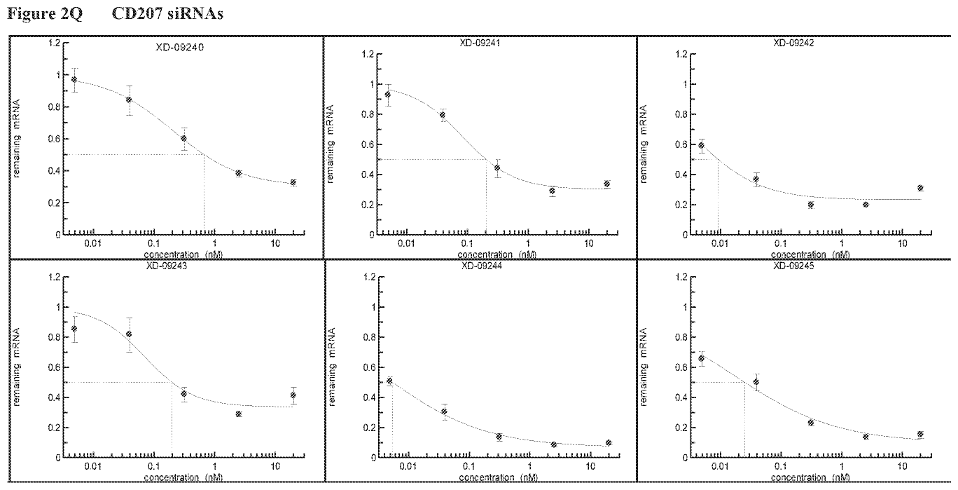

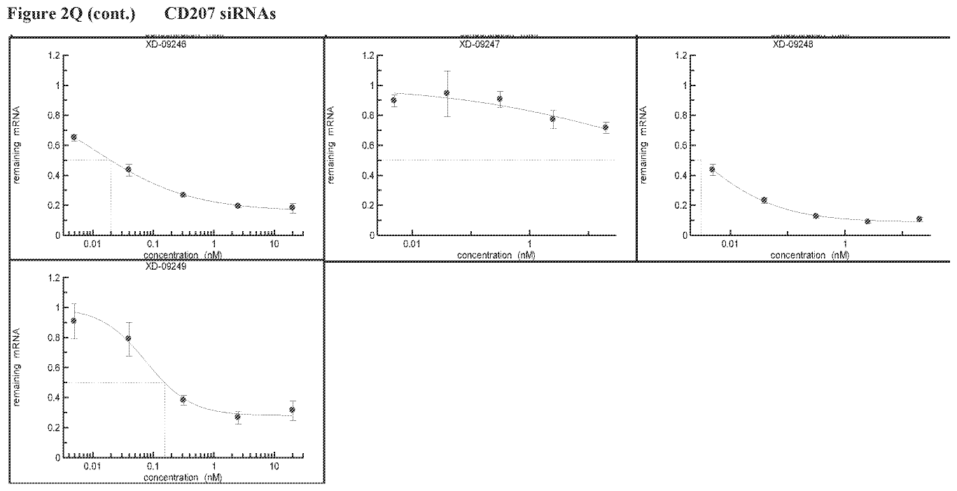

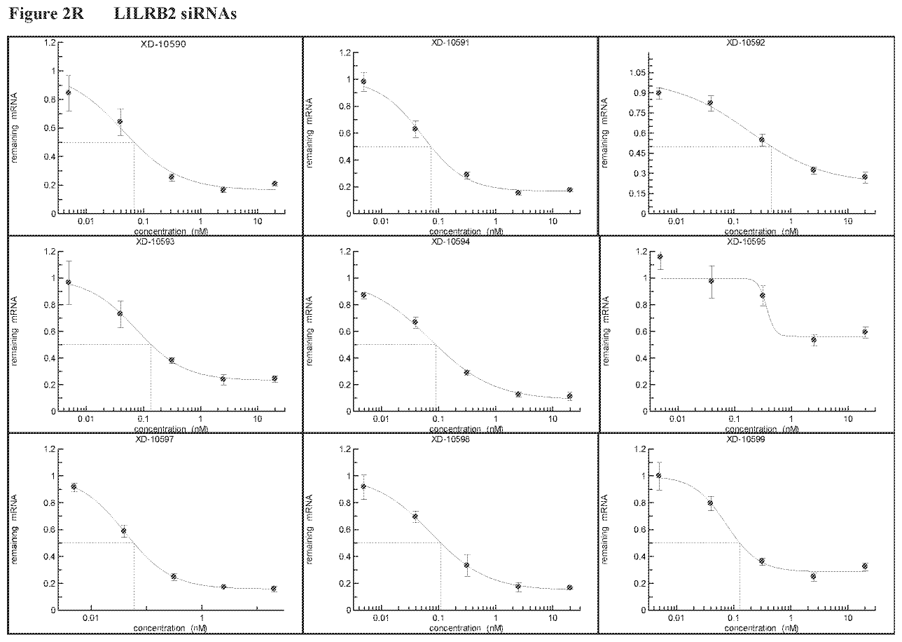

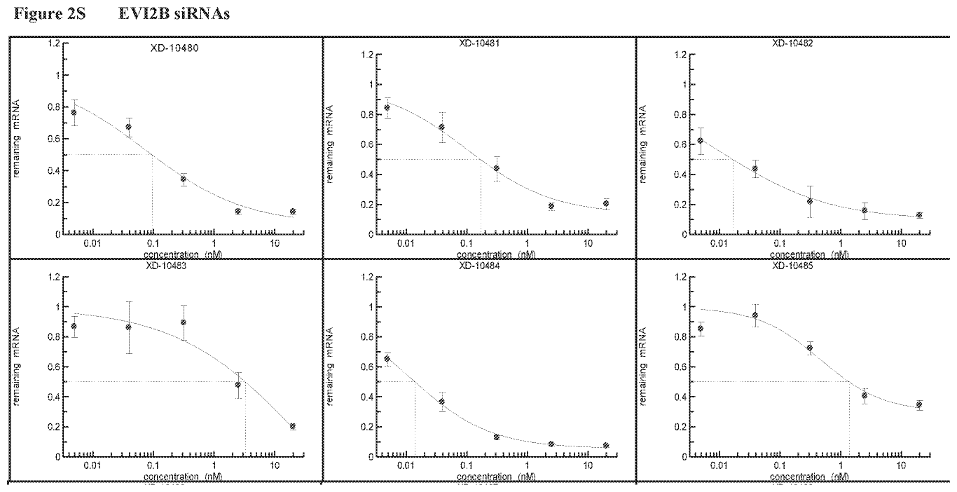

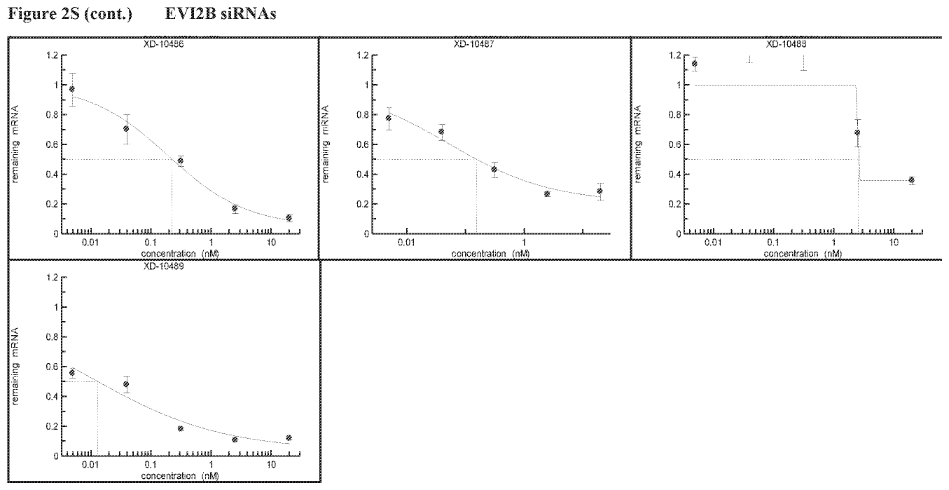

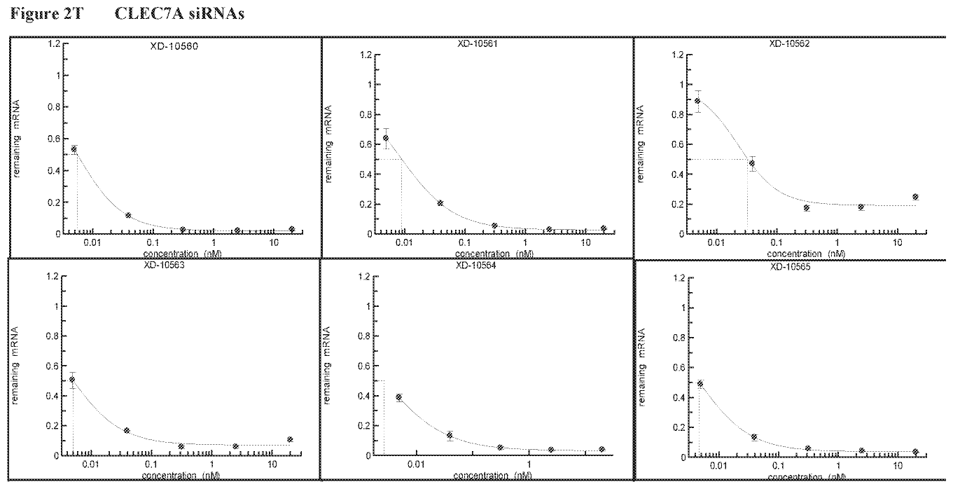

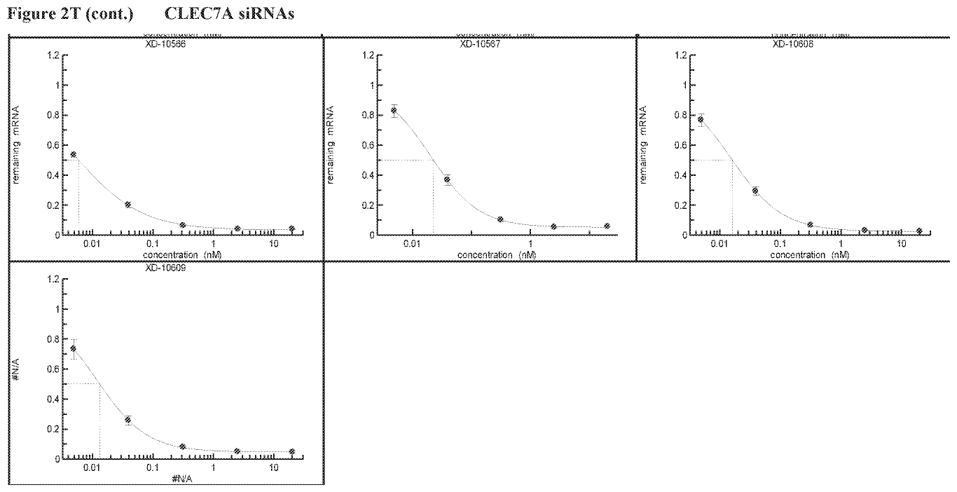

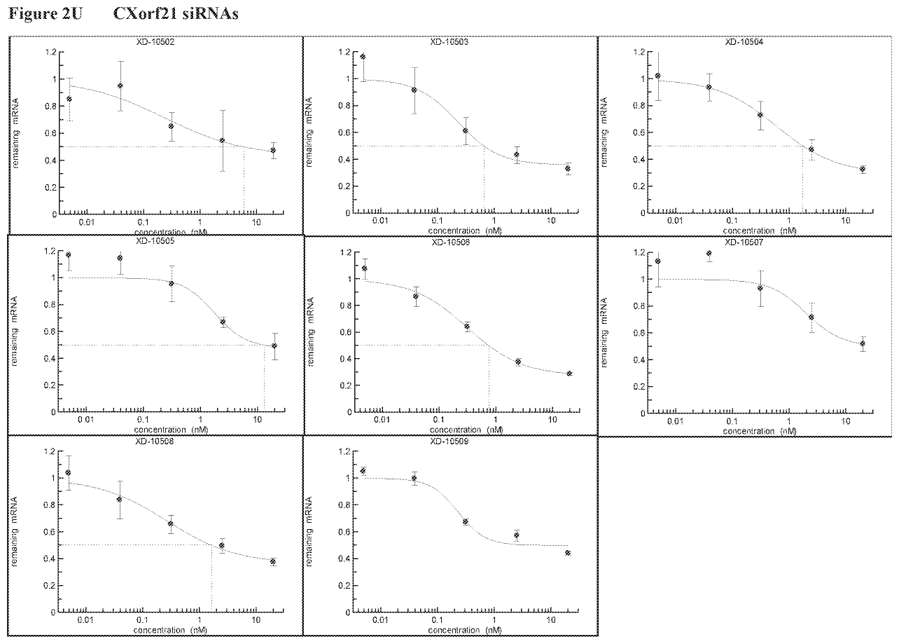

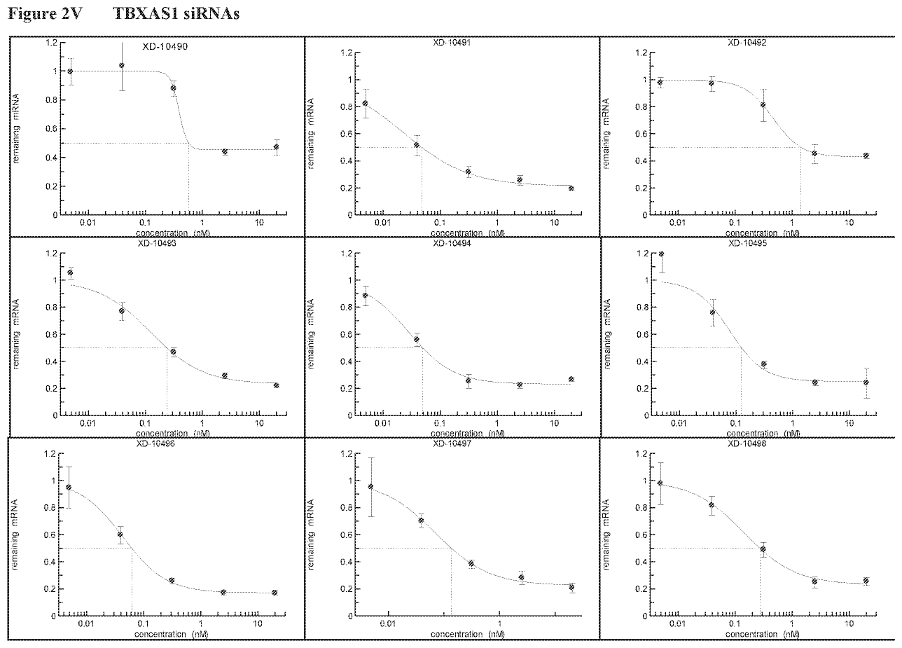

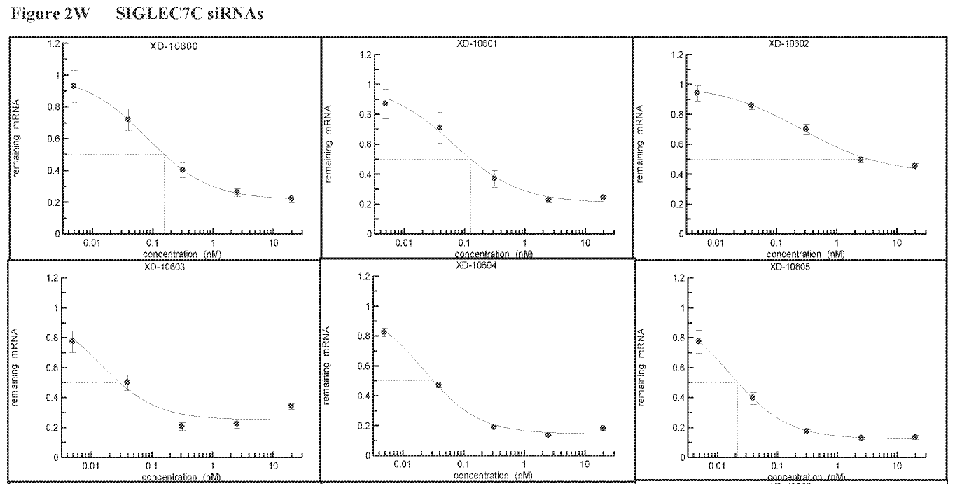

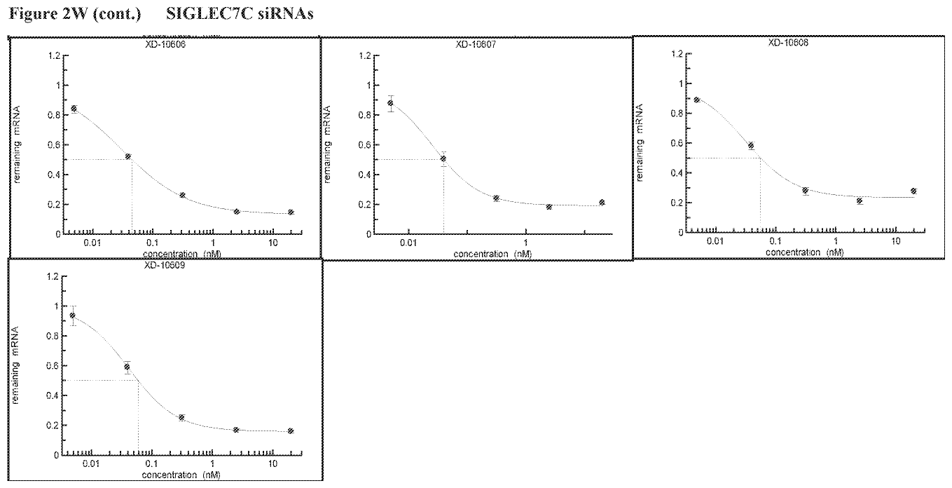

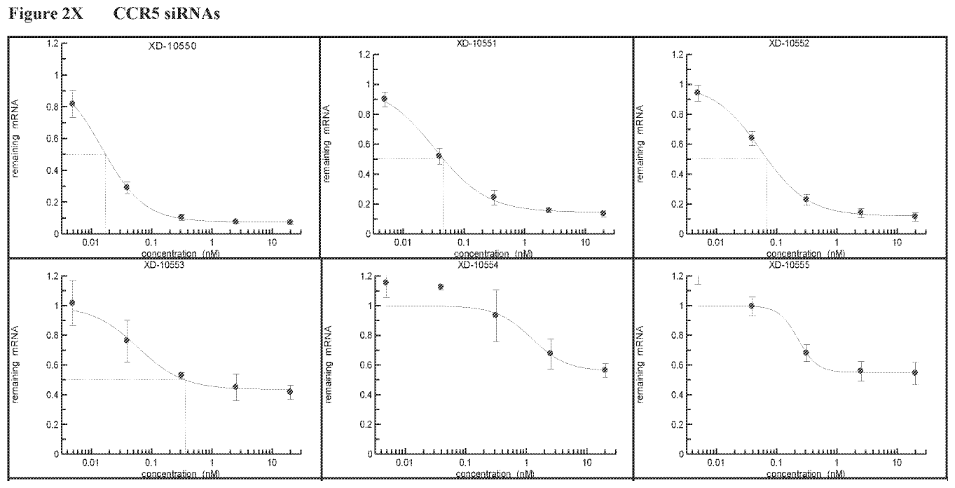

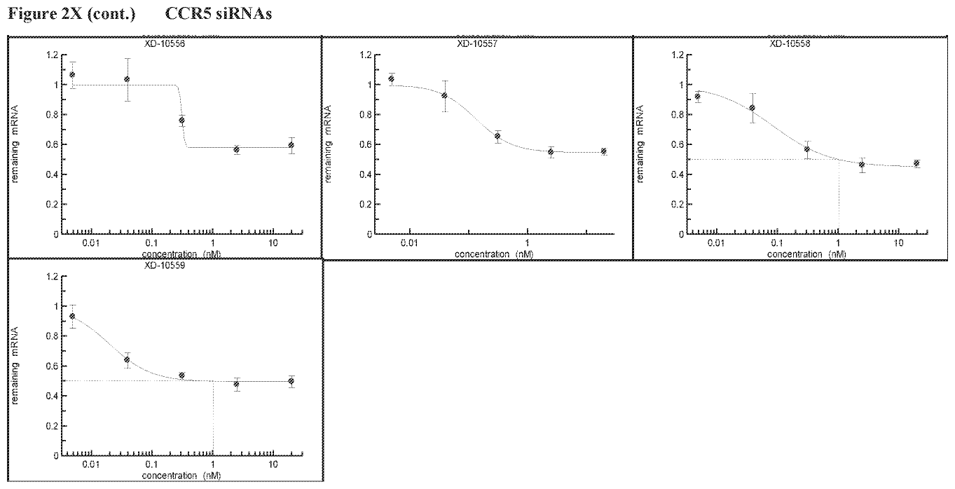

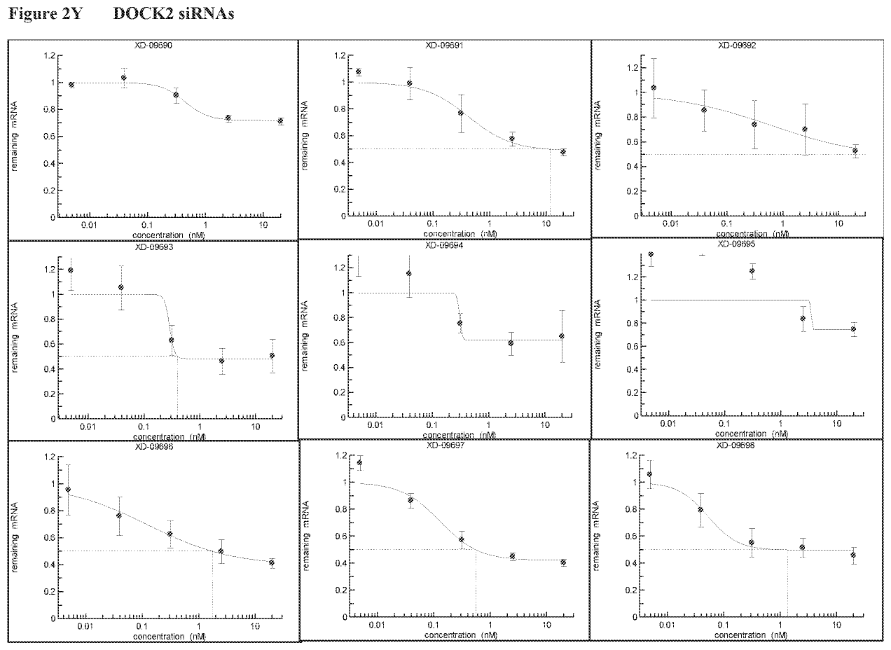

For all dose response curves, a 4-parameter logistic model was applied to determine the IC50 of each siRNA sequence (Table 5 and FIG. 2). Data shown in Table 5 and FIG. 2 represent the mean of quadruplicates, +/−standard deviation.

TABLE 5
siRNA sequences and IC50 values

| Target | siRNA ID | IC50 (nM) | Sense | Antisense |
|---|---|---|---|---|
| SIGLEC9 | XD-09180 | 0.040 | 5′-acAAGuAAAcuGcuGacGAdTsdT-3′ | 5′-UCGUcAGcAGUUuACUUGUdTsdT-3′ |
| SIGLEC9 | XD-09181 | 0.286 | 5′-guAAAcuGcuGAcgAuGcAdTsdT-3′ | 5′-UGcAUCGUcAGcAGUUuACdTsdT-3′ |
| SIGLEC9 | XD-09182 | 0.837 | 5′-ucGcAuGGcuGGAuuuAccdTsdT-3′ | 5′-GGuAAAUCcAGCcAUGCGAdTsdT-3′ |
| SIGLEC9 | XD-09183 | 0.229 | 5′-guuccGGGAAGGGgccAAudTsdT-3′ | 5′-AUUGGCCCCUUCCCGGAACdTsdT-3′ |
| SIGLEC9 | XD-09184 | 0.127 | 5′-ggGccAAuAcAGAccAGGAdTsdT-3′ | 5′-UCCUGGUCUGuAUUGGCCCdTsdT-3′ |
| SIGLEC9 | XD-09185 | 0.094 | 5′-ccAGAAGAAGuGAuGcGGGdTsdT-3′ | 5′-CCCGcAUcACUUCUUCUGGdTsdT-3′ |
| SIGLEC9 | XD-09186 | 0.488 | 5′-ucAccGGcucucuguGAAudTsdT-3′ | 5′-AUUcAcAGAGAGCCGGUGAdTsdT-3′ |
| SIGLEC9 | XD-09187 | 0.406 | 5′-gaccAcGaacAAGaccGucdTsdT-3′ | 5′-GACGGUCUUGUUCGUGGUCdTsdT-3′ |
| SIGLEC9 | XD-09188 | 0.148 | 5′-gaAcAAGAccGuccAucucdTsdT-3′ | 5′-GAGAUGGACGGUCUUGUUCdTsdT-3′ |
| SIGLEC9 | XD-09189 | 0.694 | 5′-agAccGuccAucucAAcGudTsdT-3′ | 5′-ACGUUGAGAUGGACGGUCUdTsdT-3′ |
| SIGLEC9 | XD-09190 | 0.905 | 5′-ccGuccAucucAAcGuGucdTsdT-3′ | 5′-GAcACGUUGAGAUGGACGGdTsdT-3′ |
| SIGLEC9 | XD-09191 | n.d. | 5′-ggAGAcGGcAcAGuAuccAdTsdT-3′ | 5′-UGGAuACUGUGCCGUCUCCdTsdT-3′ |
| SIGLEC9 | XD-09192 | 0.236 | 5′-caGuAuccAcAGucuuGGGdTsdT-3′ | 5′-CCcAAGACUGUGGAuACUGdTsdT-3′ |
| SIGLEC9 | XD-09193 | 0.459 | 5′-guAuccAcAGucuuGGGAAdTsdT-3′ | 5′-UUCCcAAGACUGUGGAuACdTsdT-3′ |
| SIGLEC9 | XD-09194 | 5.626 | 5′-cucAGcAGGucuAccuGAAdTsdT-3′ | 5′-UUcAGGuAGACCUGCUGAGdTsdT-3′ |
| SIGLEC9 | XD-09195 | 0.125 | 5′-gcGuGGGAGAuAcgGGcAudTsdT-3′ | 5′-AUGCCCGuAUCUCCcACGCdTsdT-3′ |
| SIGLEC9 | XD-09196 | n.d. | 5′-ugGGAGAuAcGGGcAuAGAdTsdT-3′ | 5′-UCuAUGCCCGuAUCUCCcAdTsdT-3′ |
| SIGLEC9 | XD-09197 | 0.851 | 5′-ggAGAuAcGGGcAuAGAGGdTsdT-3′ | 5′-CCUCuAUGCCCGuAUCUCCdTsdT-3′ |
| SIGLEC9 | XD-09198 | n.d. | 5′-gauAcGGGcAuAGaGGAuGdTsdT-3′ | 5′-cAUCCUCuAUGCCCGuAUCdTsdT-3′ |
| SIGLEC9 | XD-09199 | n.d. | 5′-acGGGcAuAGAGGauGcAAdTsdT-3′ | 5′-UUGCAUCCUCuAUGCCCGudTsdT-3′ |
| CD74 | XD-09223 | 0.340 | 5′-ggAGcuGucGGGAaGAucAdTsdT-3′ | 5′-UGAUCUUCCCGAcAGCUCCdTsdT-3′ |
| CD74 | XD-09224 | 1.983 | 5′-gcGcGAccuuAucuccAAcdTsdT-3′ | 5′-GUUGGAGAuAAGGUCGCGCdTsdT-3′ |
| CD74 | XD-09225 | n.d. | 5′-gcGAccuuAucuccAAcAAdTsdT-3′ | 5′-UUGUUGGAGAuAAGGUCGCdTsdT-3′ |
| CD74 | XD-09226 | 0.143 | 5′-cuGGAcAAAcuGAcAGucAdTsdT-3′ | 5′-AGACUGUcAGUUUGUCcAGdTsdT-3′ |
| CD74 | XD-09227 | 0.583 | 5′-ggAAcuGGAGGAcccGucudTsdT-3′ | 5′-AGACGGGUCCUCcAGUUCCdTsdT-3′ |
| CD74 | XD-09228 | n.d. | 5′-gcAGAGGcGGucuucAAcAdTsdT-3′ | 5′-UGUUGAAGACCGCCUCUGCdTsdT-3′ |
| CD74 | XD-09229 | n.d. | 5′-uguAccucAucccauGAGAdTsdT-3′ | 5′-UCUcAUGGGAUGAGGuAcAdTsdT-3′ |
| CD74 | XD-09230 | 0.196 | 5′-gacAAAccAAGucgGaacAdTsdT-3′ | 5′-UGUUCCGACUUGGUUUGUCdTsdT-3′ |
| CD74 | XD-09231 | 0.255 | 5′-aaAccAAGucGGAacAGcAdTsdT-3′ | 5′-UGCUGUUCCGACUUGGUUUdTsdT-3′ |
| CD74 | XD-09232 | 0.747 | 5′-agcuAGAcAGAuccccGuudTsdT-3′ | 5′-AACGGGGAUCUGUCuAGCUdTsdT-3′ |
| CD74 | XD-09233 | n.d. | 5′-cuAGGcucAuGGAcGAGAudTsdT-3′ | 5′-AUCUCGUCcAUGAGCCuAGdTsdT-3′ |
| CD74 | XD-09234 | n.d. | 5′-gaGAAGGGAuAAcccuAcAdTsdT-3′ | 5′-UGuAGGGUuAUCCCUUCUCdTsdT-3′ |
| CD74 | XD-09235 | 0.245 | 5′-ccAAuGuuuuccAcccAuAdTsdT-3′ | 5′-uAUGGGUGGAAAAcAUUGGdTsdT-3′ |
| CD74 | XD-09236 | n.d. | 5′-guuuuccAcccAuaAuccudTsdT-3′ | 5′-AGGAUuAUGGGUGGAAAACdTsdT-3′ |
| CD74 | XD-09237 | 1.841 | 5′-ccAuAAuccuuucuGccGAdTsdT-3′ | 5′-UCGGcAGAAAGGAUuAUGGdTsdT-3′ |
| CD74 | XD-09238 | n.d. | 5′-ccAAGcuuGuuAucAGcuudTsdT-3′ | 5′-AAGCUGAuAAcAAGCUUGGdTsdT-3′ |
| CD74 | XD-09239 | 0.070 | 5′-ggccAuGGuucAcauuAGAdTsdT-3′ | 5′-UCuAAUGUGAACcAUGGCCdTsdT-3′ |
| CD53 | XD-09200 | n.d. | 5′-ccGGAuAucuGuGuuAccAdTsdT-3′ | 5′-UGGuAAcAcAGAuAUCCGGdTsdT-3′ |
| CD53 | XD-09201 | 0.067 | 5′-gcAuGAGuAGcuugAAAcudTsdT-3′ | 5′-AGUUUcAAGCuACUcAUGCdTsdT-3′ |
| CD53 | XD-09203 | 0.178 | 5′-gcAuGGGcucuAucAAGGAdTsdT-3′ | 5′-UCCUUGAuAGAGCCcAUGCdTsdT-3′ |
| CD53 | XD-09205 | 0.132 | 5′-acuAAuAucuGAGcAucuudTsdT-3′ | 5′-AAGAUGCUcAGAuAUuAGUdTsdT-3′ |
| CD53 | XD-09206 | n.d. | 5′-ggAuuuAAuGGcccAAcAudTsdT-3′ | 5′-AUGUUGGGCcAUuAAAUCCdTsdT-3′ |
| CD53 | XD-09207 | 0.038 | 5′-agGGcAAGAucucauuucAdTsdT-3′ | 5′-UGAAAUGAGAUCUUGCCCUdTsdT-3′ |
| CD53 | XD-09208 | 0.162 | 5′-uuuAuuAGAGGGccuuAuudTsdT-3′ | 5′-AAuAAGGCCCUCuAAuAAAdTsdT-3′ |
| CD53 | XD-09209 | 0.041 | 5′-auuGAuGuGuucuaAGucudTsdT-3′ | 5′-AGACUuAGAAcAcAUcAAUdTsdT-3′ |
| SELPLG | XD-09260 | n.d. | 5′-ggGcAGAuGAAGccGAGAAdTsdT-3′ | 5′-UUCUCGGCUUcAUCUGCCCdTsdT-3′ |
| SELPLG | XD-09261 | 0.386 | 5′-ccAccGAAuAUGAguAccudTsdT-3′ | 5′-AGGuACUcAuAUUCGGUGGdTsdT-3′ |
| SELPLG | XD-09262 | 1.354 | 5′-ccGAAuAuGAGuAccuAGAdTsdT-3′ | 5′-UCuAGGuACUcAuAUUCGGdTsdT-3′ |
| SELPLG | XD-09263 | 2.964 | 5′-gcAcAGAccAcucaAcccAdTsdT-3′ | 5′-UGGGUUGAGUGGUCUGUGCdTsdT-3′ |
| SELPLG | XD-09264 | 1.963 | 5′-accAAAAGAGGucuGuucAdTsdT-3′ | 5′-UGAAcAGACCUCUUUUGGUdTsdT-3′ |
| SELPLG | XD-09265 | 0.486 | 5′-aaGcAGAcGGGcAaGuGGAdTsdT-3′ | 5′-UCcACUUGCCCGUCUGCUUdTsdT-3′ |
| SELPLG | XD-09266 | 0.207 | 5′-aguAcuGAAGAGugAcGGAdTsdT-3′ | 5′-UCCGUcACUCUUcAGuACUdTsdT-3′ |
| SELPLG | XD-09267 | 1.067 | 5′-acuGAAGAGuGAcgGAcuudTsdT-3′ | 5′-AAGUCCGUcACUCUUcAGUdTsdT-3′ |
| SELPLG | XD-09268 | 0.471 | 5′-ccAAGGGcAGAccuuucuudTsdT-3′ | 5′-AAGAAAGGUCUGCCCUUGGdTsdT-3′ |
| VSIG4 | XD-09170 | 0.024 | 5′-aaGGcuAcAcccAaGucuudTsdT-3′ | 5′-AAGACUUGGGUGuAGCCUUdTsdT-3′ |
| VSIG4 | XD-09171 | 0.023 | 5′-cuuucuAcGuGAcucuucudTsdT-3′ | 5′-AGAAGAGUcACGuAGAAAGdTsdT-3′ |
| VSIG4 | XD-09172 | <0.005 | 5′-gcAAccAAGucGugAGAGAdTsdT-3′ | 5′-UCUCUcACGACUUGGUUGCdTsdT-3′ |
| VSIG4 | XD-09173 | <0.005 | 5′-cguGAGAGAuAAGauuAcudTsdT-3′ | 5′-AGuAAUCUuAUCUCUcACGdTsdT-3′ |
| VSIG4 | XD-09174 | 0.036 | 5′-aacAAGAGcAuGucuAcGAdTsdT-3′ | 5′-UCGuAGAcAUGCUCUUGUUdTsdT-3′ |
| VSIG4 | XD-09176 | 0.031 | 5′-cacuAGGAcuuGGucAucAdTsdT-3′ | 5′-AGAUGACcAAGUCCuAGUGdTsdT-3′ |
| VSIG4 | XD-09177 | 0.007 | 5′-acuAGGAcuuGGucAucAudTsdT-3′ | 5′-AUGAUGACcAAGUCCuAGUdTsdT-3′ |
| VSIG4 | XD-09178 | 0.060 | 5′-gucAucAuGccuAcAGAcAdTsdT-3′ | 5′-UGUCUGuAGGcAUGAUGACdTsdT-3′ |
| VSIG4 | XD-09179 | 0.006 | 5′-ggccAGAcAGcuuuuAAuudTsdT-3′ | 5′-AAUuAAAAGCUGUCUGGCCdTsdT-3′ |
| LRRC25 | XD-09250 | 14.978 | 5′-gccAGAGGAAcGccAGcGAdTsdT-3′ | 5′-UCGCUGGCGUUCCUCUGGCdTsdT-3′ |
| LRRC25 | XD-09251 | 2.770 | 5′-gccGucGcuuuuccAGcAudTsdT-3′ | 5′-AUGCUGGAAAAGCGACGGCdTsdT-3′ |
| LRRC25 | XD-09252 | n.d. | 5′-ggAAcGGccuGcGaGAGcudTsdT-3′ | 5′-AGCUCUCGcAGGCCGUUCCdTsdT-3′ |
| LRRC25 | XD-09256 | 0.601 | 5′-ccGAcuAuGAGAAcAuGuudTsdT-3′ | 5′-AAcAUGUUCUcAuAGUCGGdTsdT-3′ |
| LRRC25 | XD-09257 | n.d. | 5′-gccuAAGAuGuccuAAccudTsdT-3′ | 5′-AGGUuAGGAcAUCUuAGGCdTsdT-3′ |
| LRRC25 | XD-09258 | 0.033 | 5′-caAuAAAuGcuucgAGGcudTsdT-3′ | 5′-AGCCUCGAAGcAUUuAUUGdTsdT-3′ |
| LRRC25 | XD-09259 | 0.293 | 5′-cuucGAGGcuGAugAGGcudTsdT-3′ | 5′-AGCCUcAUcAGCCUCGAAGdTsdT-3′ |
| AIF1 | XD-09660 | n.d. | 5′-cuuAAuGGAAAuGgcGAuAdTsdT-3′ | 5′-uAUCGCcAUUUCcAUuAAGdTsdT-3′ |
| AIF1 | XD-09661 | 0.045 | 5′-uaAuGGAAAuGGcgAuAuudTsdT-3′ | 5′-AAuAUCGCcAUUUCcAUuAdTsdT-3′ |
| AIF1 | XD-09662 | 0.120 | 5′-uaucAuGucccuGaAAcGAdTsdT-3′ | 5′-UCGUUUcAGGGAcAUGAuAdTsdT-3′ |
| AIF1 | XD-09663 | 0.263 | 5′-gaAAcGAAuGcuGgAGAAAdTsdT-3′ | 5′-UUUCUCcAGcAUUCGUUUCdTsdT-3′ |
| AIF1 | XD-09664 | 0.433 | 5′-agAcucAccuAGAgcuAAAdTsdT-3′ | 5′-UUuAGCUCuAGGUGAGUCUdTsdT-3′ |
| AIF1 | XD-09665 | 0.349 | 5′-cucAccuAGAGcuaAAGAAdTsdT-3′ | 5′-UUCUUuAGCUCuAGGUGAGdTsdT-3′ |
| AIF1 | XD-09666 | 0.105 | 5′-ucAccuAGAGcuAaAGAAAdTsdT-3′ | 5′-UUUCUUuAGCUCuAGGUGAdTsdT-3′ |
| AIF1 | XD-09667 | 0.042 | 5′-accuAGAGcuAAAgAAAuudTsdT-3′ | 5′-AAUUUCUUuAGCUCuAGGUdTsdT-3′ |
| AIF1 | XD-09668 | 0.267 | 5′-ccuAGAGcuAAAGaAAuuAdTsdT-3′ | 5′-uAAUUUCUUuAGCUCuAGGdTsdT-3′ |
| AIF1 | XD-09669 | 1.234 | 5′-ccGGGGAGAcGuucAGcuAdTsdT-3′ | 5′-uAGCUGAACGUCUCCCCGGdTsdT-3′ |
| CD84 | XD-09610 | 0.047 | 5′-accAAAAAuuAcAcAGAGudTsdT-3′ | 5′-ACUCUGUGuAAUUUUUGGUdTsdT-3′ |
| CD84 | XD-09611 | 0.082 | 5′-gaGGGuAAuGuccuucAAAdTsdT-3′ | 5′-UUUGAAGGAcAUuACCCUCdTsdT-3′ |
| CD84 | XD-09612 | 0.351 | 5′-gguAAuGuccuucaAAucudTsdT-3′ | 5′-AGAUUUGAAGGAcAUuACCdTsdT-3′ |
| CD84 | XD-09613 | 0.241 | 5′-ucAuAuGAucAuuuAAGGAdTsdT-3′ | 5′-UCCUuAAAUGAUcAuAUGAdTsdT-3′ |
| CD84 | XD-09614 | <0.005 | 5′-gaAcAcAGuuuAuuccGAAdTsdT-3′ | 5′-UUCGGAAuAAACUGUGUUCdTsdT-3′ |
| CD84 | XD-09615 | 0.048 | 5′-ggAAcuGGGuAuAauccAAdTsdT-3′ | 5′-UUGGAUuAuACCcAGUUCCdTsdT-3′ |
| CD84 | XD-09616 | 0.067 | 5′-gcAccAAcuAccAuuAGcAdTsdT-3′ | 5′-AGCuAAUGGuAGUUGGUGCdTsdT-3′ |
| CD84 | XD-09617 | 0.010 | 5′-cacuAcAGuuAAcgcuGAAdTsdT-3′ | 5′-UUcAGCGUuAACUGuAGUGdTsdT-3′ |
| CD84 | XD-09618 | <0.005 | 5′-gguAucAGGAAucaAAAuudTsdT-3′ | 5′-AAUUUUGAUUCCUGAuACCdTsdT-3′ |
| CD84 | XD-09619 | <0.005 | 5′-ccuuAcAGcAuAAcuAAuudTsdT-3′ | 5′-AAUuAGUuAUGCUGuAAGGdTsdT-3′ |
| IGSF6 | XD-09641 | n.d. | 5′-gaGGccGucAccAuAAAGudTsdT-3′ | 5′-ACUUuAUGGUGACGGCCUCdTsdT-3′ |
| IGSF6 | XD-09642 | 0.035 | 5′-gaAGcGAGAGcuAaAcAGAdTsdT-3′ | 5′-UCUGUUuAGCUCUCGCUUCdTsdT-3′ |
| IGSF6 | XD-09643 | 0.077 | 5′-guGcucGGcGuAuuuuucAdTsdT-3′ | 5′-UGAAAAAuACGCCGAGcACdTsdT-3′ |
| IGSF6 | XD-09645 | 0.034 | 5′-agAAcuAuAccAuaAGAGAdTsdT-3′ | 5′-UCUCUuAUGGuAuAGUUCUdTsdT-3′ |
| IGSF6 | XD-09646 | 0.010 | 5′-ccAuAAGAGAcAuguGGAAdTsdT-3′ | 5′-UUCcAcAUGUCUCUuAUGGdTsdT-3′ |
| IGSF6 | XD-09647 | 0.069 | 5′-ggccAuAGAAAcGuuuuAAdTsdT-3′ | 5′-UuAAAACGUUUCuAUGGCCdTsdT-3′ |
| IGSF6 | XD-09648 | 0.503 | 5′-ccAuAGAAAcGuuuuAAuudTsdT-3′ | 5′-AAUuAAAACGUUUCuAUGGdTsdT-3′ |
| IGSF6 | XD-09649 | 0.503 | 5′-acAuAuAucAucAgGucuudTsdT-3′ | 5′-AAGACCUGAUGAuAuAUGUdTsdT-3′ |
| FERMT3 | XD-09700 | n.d. | 5′-cgAcGcAcGccucuucuuudTsdT-3′ | 5′-AAAGAAGAGGCGUGCGUCGdTsdT-3′ |
| FERMT3 | XD-09701 | 0.921 | 5′-ggcuGcGcuucAAguAcuAdTsdT-3′ | 5′-uAGuACUUGAAGCGcAGCCdTsdT-3′ |
| FERMT3 | XD-09705 | 6.753 | 5′-guucccGAuGuuAacGucudTsdT-3′ | 5′-AGACGUuAAcAUCGGGAACdTsdT-3′ |
| FERMT3 | XD-09707 | n.d. | 5′-ccAGcuGccGAAuuGuAcAdTsdT-3′ | 5′-UGuAcAAUUCGGcAGCUGGdTsdT-3′ |
| FERMT3 | XD-09708 | 0.012 | 5′-gcuGccGAAuuGuacAcGAdTsdT-3′ | 5′-UCGUGuAcAAUUCGGcAGCdTsdT-3′ |
| FERMT3 | XD-09709 | 0.048 | 5′-guAGcuAcAGGAugAuGAAdTsdT-3′ | 5′-UUcAUcAUCCUGuAGCuACdTsdT-3′ |
| CD48 | XD-09720 | 0.012 | 5′-cucuGGAAuuGcuacuGcudTsdT-3′ | 5′-AGcAGuAGcAAUUCcAGAGdTsdT-3′ |
| CD48 | XD-09721 | 0.009 | 5′-cuAAccuGGuuuuauAcuudTsdT-3′ | 5′-AAGuAuAAAACcAGGUuAGdTsdT-3′ |
| CD48 | XD-09722 | 0.014 | 5′-cuuucGAccAGAAgAuuGudTsdT-3′ | 5′-AcAAUCUUCUGGUCGAAAGdTsdT-3′ |
| CD48 | XD-09723 | 0.030 | 5′-uccAGAAAAucuAaGuAcudTsdT-3′ | 5′-AGuACUuAGAUUUUCUGGAdTsdT-3′ |
| CD48 | XD-09724 | 0.055 | 5′-gaAuccAAAuuuAaAGGcAdTsdT-3′ | 5′-UGCCUUuAAAUUUGGAUUCdTsdT-3′ |
| CD48 | XD-09725 | 0.022 | 5′-cccAAGccuGucAucAAAAdTsdT-3′ | 5′-UUUUGAUGAcAGGCUUGGGdTsdT-3′ |
| CD48 | XD-09726 | 0.017 | 5′-acAAcuGuuAucugAAAcudTsdT-3′ | 5′-AGUUUcAGAuAAcAGUUGUdTsdT-3′ |
| CD48 | XD-09727 | 0.046 | 5′-ccuGGcGAGucuGuAAAcudTsdT-3′ | 5′-AGUUuAcAGACUCGCcAGGdTsdT-3′ |
| CD48 | XD-09728 | 0.032 | 5′-cuGGcGAGucuGuaAAcuAdTsdT-3′ | 5′-UagUUuAcAGACUCGCcAGdTsdT-3′ |
| CD48 | XD-09729 | 0.015 | 5′-uguuAuAcuuGccaAGucAdTsdT-3′ | 5′-UGACUUGGcAAGuAuAAcAdTsdT-3′ |
| CD33 | XD-09731 | n.d. | 5′-ccGGccAcuccAAaAAccudTsdT-3′ | 5′-AGGUUUUUGGAGUGGCCGGdTsdT-3′ |
| CD33 | XD-09732 | 5.400 | 5′-ggccccAGGAcuAcucAcudTsdT-3′ | 5′-AGUGAGuAGUCCUGGGGCCdTsdT-3′ |
| CD33 | XD-09734 | 0.740 | 5′-acuccucGGuGcucAuAAudTsdT-3′ | 5′-AUuAUGAGcACCGAGGAGUdTsdT-3′ |
| CD33 | XD-09735 | 0.818 | 5′-caAcGucAccuAuguuccAdTsdT-3′ | 5′-UGGAAcAuAGGUGACGUUGdTsdT-3′ |
| CD33 | XD-09736 | 0.176 | 5′-ucAccuAuGuuccacAGAAdTsdT-3′ | 5′-UUCUGUGGAAcAuAGGUGAdTsdT-3′ |
| CD33 | XD-09737 | 0.334 | 5′-cccAAcAAcuGGuaucuuudTsdT-3′ | 5′-AAAGAuACcAGUUGUUGGGdTsdT-3′ |
| CD33 | XD-09738 | 0.220 | 5′-ccAGAGcAGGAGugGuucAdTsdT-3′ | 5′-UGAACcACUCCUGCUCUGGdTsdT-3′ |
| CD33 | XD-09739 | n.d. | 5′-gguucAuGGGGccauuGGAdTsdT-3′ | 5′-UCcAAUGGCCCcAUGAACCdTsdT-3′ |
| LST1 | XD-09750 | 3.594 | 5′-cuGAucAuuucGccuAAAAdTsdT-3′ | 5′-UUUuAGGCGAAAUGAUcAGdTsdT-3′ |
| LST1 | XD-09751 | 1.539 | 5′-cauuucGccuAAAaGAGcAdTsdT-3′ | 5′-UGCUCUUUuAGGCGAAAUGdTsdT-3′ |
| LST1 | XD-09752 | 0.737 | 5′-gccuAAAAGAGcAaGGAcudTsdT-3′ | 5′-AGUCCUUGCUCUUUuAGGCdTsdT-3′ |
| LST1 | XD-09753 | n.d. | 5′-ggAAcuccAcuAugcAucudTsdT-3′ | 5′-AGAUGcAuAGUGGAGUUCCdTsdT-3′ |
| LST1 | XD-09754 | 0.052 | 5′-caAGGAGGAuccAaGAGcudTsdT-3′ | 5′-AGCUCUUGGAUCCUCCUUGdTsdT-3′ |
| LST1 | XD-09755 | 0.046 | 5′-ggAuccAAGAGcugAcuAudTsdT-3′ | 5′-AuAGUcAGCUCUUGGAUCCdTsdT-3′ |
| LST1 | XD-09756 | 6.598 | 5′-ucucGAGccuccGuucAAAdTsdT-3′ | 5′-UUUGAACGGAGGCUCGAGAdTsdT-3′ |
| LST1 | XD-09757 | n.d. | 5′-cuccGuucAAAuugAucAudTsdT-3′ | 5′-AUGAUcAAUUUGAACGGAGdTsdT-3′ |
| LST1 | XD-09758 | 8.205 | 5′-ugAucAucAucAAaAcuuAdTsdT-3′ | 5′-uAAGUUUUGAUGAUGAUcAdTsdT-3′ |
| LST1 | XD-09759 | n.d. | 5′-accuuuGAAuAGGgAAuuudTsdT-3′ | 5′-AAAUUCCCuAUUcAAAGGUdTsdT-3′ |
| TNFAIP8L2 | XD-09760 | 0.058 | 5′-ggAccAGGccGuGaucucudTsdT-3′ | 5′-AGAGAUcACGGCCUGGUCCdTsdT-3′ |
| TNFAIP8L2 | XD-09761 | 0.701 | 5′-ggucAGGuGGAGAucucuudTsdT-3′ | 5′-AAGAGAUCUCcACCUGACCdTsdT-3′ |
| TNFAIP8L2 | XD-09762 | 0.226 | 5′-uuuAAuGuAucGcuAccAAdTsdT-3′ | 5′-UUGGuAGCGAuAcAUuAAAdTsdT-3′ |
| TNFAIP8L2 | XD-09763 | n.d. | 5′-aaAucGAcAccuGaAAAAGdTsdT-3′ | 5′-CUUUUUcAGGUGUCGAUUUdTsdT-3′ |
| TNFAIP8L2 | XD-09764 | 2.979 | 5′-cguuGcGcuGGAAgGAAuudTsdT-3′ | 5′-AAUUCCUUCcAGCGcAACGdTsdT-3′ |
| TNFAIP8L2 | XD-09765 | 0.023 | 5′-cuGcGcGAGGcGcaAuAcAdTsdT-3′ | 5′-UGuAUUGCGCCUCGCGcAGdTsdT-3′ |
| TNFAIP8L2 | XD-09766 | 0.127 | 5′-cccucGAuGuAcAuAGcuudTsdT-3′ | 5′-AAGCuAUGuAcAUCGAGGGdTsdT-3′ |
| TNFAIP8L2 | XD-09767 | n.d. | 5′-cacAGuGcAuAAGcAGuuudTsdT-3′ | 5′-AAACUGCUuAUGcACUGUGdTsdT-3′ |
| TNFAIP8L2 | XD-09768 | 0.353 | 5′-aaAucAAucAuGuuAcAcudTsdT-3′ | 5′-AGUGuAAcAUGAUUGAUUUdTsdT-3′ |
| TNFAIP8L2 | XD-09796 | 11.602 | 5′-gcAcAuAGcAAGcuGAAcudTsdT-3′ | 5′-AGUUcAGCUUGCuAUGUGCdTsdT-3′ |
| CD37 | XD-09770 | 0.553 | 5′-gcuuGcGGGAcGucGuAGAdTsdT-3′ | 5′-UCuACGACGUCCCGcAAGCdTsdT-3′ |
| CD37 | XD-09771 | 0.388 | 5′-gacGucGuAGAGAaAAccAdTsdT-3′ | 5′-UGGUUUUCUCuACGACGUCdTsdT-3′ |
| CD37 | XD-09773 | 0.311 | 5′-cgAccAAcGAcuccAcAAudTsdT-3′ | 5′-AUUGUGGAGUCGUUGGUCGdTsdT-3′ |
| CD37 | XD-09774 | 0.129 | 5′-ccAAcGAcuccAcaAuccudTsdT-3′ | 5′-AGGAUUGUGGAGUCGUUGGdTsdT-3′ |
| CD37 | XD-09775 | 1.600 | 5′-ccAcAAuccuAGAuAAGGudTsdT-3′ | 5′-ACCUuAUCuAGGAUUGUGGdTsdT-3′ |
| CD37 | XD-09776 | 1.320 | 5′-ggcuGcAcAAcAAccuuAudTsdT-3′ | 5′-AuAAGGUUGUUGUGcAGCCdTsdT-3′ |
| CD37 | XD-09777 | 0.414 | 5′-guucAuGAcGcucucGAuAdTsdT-3′ | 5′-uAUCGAGAGCGUcAUGAACdTsdT-3′ |
| CD37 | XD-09778 | 11.400 | 5′-cgGcucGcucGAuaccGuudTsdT-3′ | 5′-AACGGuAUCGAGCGAGCCGdTsdT-3′ |
| CD37 | XD-09779 | <0.005 | 5′-accAcccAcAAGAuuAuuudTsdT-3′ | 5′-AAAuAAUCUUGUGGGUGGUdTsdT-3′ |
| SPI1 | XD-09780 | 2.663 | 5′-ggAucuAuAccAAcGccAAdTsdT-3′ | 5′-UUGGCGUUGGuAuAGAUCCdTsdT-3′ |
| SPI1 | XD-09781 | 0.455 | 5′-cgccAAAcGcAcGaGuAuudTsdT-3′ | 5′-AAuACUCGUGCGUUUGGCGdTsdT-3′ |
| SPI1 | XD-09782 | n.d. | 5′-gcuucGccGAGAAcAAcuudTsdT-3′ | 5′-AAGUUGUUCUCGGCGAAGCdTsdT-3′ |
| SPI1 | XD-09783 | 1.108 | 5′-gcGAcAuGAAGGAcAGcAudTsdT-3′ | 5′-AUGCUGUCCUUcAUGUCGCdTsdT-3′ |
| SPI1 | XD-09784 | n.d. | 5′-cccGcuGGccAuAgcAuuAdTsdT-3′ | 5′-uAAUGCuAUGGCcAGCGGGdTsdT-3′ |
| SPI1 | XD-09785 | 0.479 | 5′-gucucAAGuccGuauGuaadTsdT-3′ | 5′-UuAcAuACGGACUUGAGACdTsdT-3′ |
| SPI1 | XD-09786 | 0.250 | 5′-ucucAAGuccGuAuGuAAAdTsdT-3′ | 5′-UUuAcAuACGGACUUGAGAdTsdT-3′ |
| SPI1 | XD-09787 | 0.075 | 5′-ccGuAuGuAAAucaGAucudTsdT-3′ | 5′-AGAUCUGAUUuAcAuACGGdTsdT-3′ |
| SPI1 | XD-09788 | 1.316 | 5′-acAAGuAAAGuuAuucucAdTsdT-3′ | 5′-UGAGAAuAACUUuACUUGUdTsdT-3′ |
| SPI1 | XD-09789 | 0.442 | 5′-aaAGuuAuucucAauccAudTsdT-3′ | 5′-AUGGAUUGAGAAuAACUUUdTsdT-3′ |
| CD207 | XD-09240 | 0.678 | 5′-guGAGcAcucAGGauGAcudTsdT-3′ | 5′-AGUcAUCCUGAGUGCUcACdTsdT-3′ |
| CD207 | XD-09241 | 0.203 | 5′-gaAAAcAcccAcAguccGudTsdT-3′ | 5′-ACGGACUGUGGGUGUUUUCdTsdT-3′ |
| CD207 | XD-09242 | 0.009 | 5′-caGuccGuGcuGcauuAAudTsdT-3′ | 5′-AUuAAUGcAGcACGGACUGdTsdT-3′ |
| CD207 | XD-09243 | 0.198 | 5′-guccGuGcuGcAuuAAucudTsdT-3′ | 5′-AGAUuAAUGcAGcACGGACdTsdT-3′ |
| CD207 | XD-09244 | 0.005 | 5′-ugcuGAAAGGucGuGuGGAdTsdT-3′ | 5′-UCcAcACGACCUUUcAGcAdTsdT-3′ |
| CD207 | XD-09245 | 0.020 | 5′-gcGuucAGAuccAgAuGGudTsdT-3′ | 5′-ACcAUCUGGAUCUGAACGCdTsdT-3′ |
| CD207 | XD-09246 | 0.025 | 5′-aaAuAcAAAGAuccGGGcAdTsdT-3′ | 5′-UGCCCGGAUCUUUGuAUUUdTsdT-3′ |
| CD207 | XD-09247 | n.d. | 5′-auAuGAGcAAGuugcucAAdTsdT-3′ | 5′-UUGAGcAACUUGCUcAuAUdTsdT-3′ |
| CD207 | XD-09248 | <0.005 | 5′-uauucuAcAGGuGguuucudTsdT-3′ | 5′-AGAAACcACCUGuAGAAuAdTsdT-3′ |
| CD207 | XD-09249 | 0.153 | 5′-uauAGuGccGAGcaGuucudTsdT-3′ | 5′-AGAACUGCUCGGcACuAuAdTsdT-3′ |
| LILRB2 | XD-10590 | 0.068 | 5′-caGcAuCuuGGAuuAcAcsa-3′ | 5′-dTGUGuAAUCcAAGAUGCUGusu-3′ |
| LILRB2 | XD-10591 | 0.073 | 5′-ggAuAcGAccAGAgcuugsa-3′ | 5′-dTCAAGCUCUGGUCGuAUccusu-3′ |
| LILRB2 | XD-10592 | 0.459 | 5′-gcGAuAUGGcuGucAGuasa-3′ | 5′-dTUACUGAcAGCcAUAUCGCusu-3′ |
| LILRB2 | XD-10593 | 0.136 | 5′-cuAuGGUuAuGAcuuGAasa-3′ | 5′-dTUUcAAGUcAuAACcAuAGusu-3′ |
| LILRB2 | XD-10594 | 0.089 | 5′-gguuAuGAcuuGAccucusa-3′ | 5′-dTAGAGUUcAAGUcAuAACCusu-3′ |
| LILRB2 | XD-10595 | n.d. | 5′-ggcAcACccuucAucucasa-3′ | 5′-dTUGAGAUGAAGGGUGUGCCusu-3′ |
| LILRB2 | XD-10597 | 0.059 | 5′-cccAcuCcGucuAaGAucsa-3′ | 5′-dTGAUCUuAGACGGAGUGGGusu-3′ |
| LILRB2 | XD-10598 | 0.109 | 5′-ccAcucCGucuAAgAucasa-3′ | 5′-dTUGAUCUuAGACGGAGUGGusu-3′ |
| LILRB2 | XD-10599 | 0.127 | 5′-cacuccGucuAAGaucAasa-3′ | 5′-dTUUGAUCUuAGACGGAGUGusu-3′ |
| EVI2B | XD-10480 | 0.094 | 5′-gaGAcAAuuAcAAcAGAGAdTsdT-3′ | 5′-UCUCUGUUGuAAUUGUCUDdTsdT-3′ |
| EVI2B | XD-10481 | 0.169 | 5′-cuuuGGGucAAccaAcAcAdTsdT-3′ | 5′-UGUGUUGGUUGACCcAAAGdTsdT-3′ |
| EVI2B | XD-10482 | 0.017 | 5′-caAccAAcAccAAuAGccAdTsdT-3′ | 5′-UGGCuAUUGGUGUUGGUUGdTsdT-3′ |
| EVI2B | XD-10483 | 3.272 | 5′-ccAucuGcccGuAcuucuAdTsdT-3′ | 5′-uAGAAGuACGGGcAGAUGGdTsdT-3′ |
| EVI2B | XD-10484 | 0.014 | 5′-ccAccAAAGucAuuuGucudTsdT-3′ | 5′-AGAcAAAUGACUUUGGUGGdTsdT-3′ |
| EVI2B | XD-10485 | 1.380 | 5′-gucAAAAAuucAccuAGGAdTsdT-3′ | 5′-UCCuAGGUGAAUUUUUGACdTsdT-3′ |
| EVI2B | XD-10486 | 0.225 | 5′-ggAuuuAucuuAGauAcuAdTsdT-3′ | 5′-uAGuAUCuAAGAuAAAUCCdTsdT-3′ |
| EVI2B | XD-10487 | 0.156 | 5′-cauAcuAAuuGGuguAcuudTsdT-3′ | 5′-AAGuAcACcAAUuAGuAUGdTsdT-3′ |
| EVI2B | XD-10488 | 2.552 | 5′-agcuAuAAucAucauuGuAdTsdT-3′ | 5′-uAcAAUGAUGAUuAuAGCUdTsdT-3′ |
| EVI2B | XD-10489 | 0.013 | 5′-cucccAAcucuGAucAAGAdTsdT-3′ | 5′-UCUUGAUcAGAGUUGGGAGdTsdT-3′ |
| CLEC7A | XD-10560 | 0.005 | 5′-ggAuAuAcucAAuuAcAcudTsdT-3′ | 5′-AGUGuAAUUGAGuAuAUCCdTsdT-3′ |
| CLEC7A | XD-10561 | 0.009 | 5′-ggucAAGAuAAAugcAGAAdTsdT-3′ | 5′-UUCUGcAUUaAUCUUAACCdTsdT-3′ |
| CLEC7A | XD-10562 | 0.033 | 5′-agGuAGGcuAGuAuuAuuudTsdT-3′ | 5′-AAAuAAuACuAGCCuACCUdTsdT-3′ |
| CLEC7A | XD-10563 | 0.005 | 5′-ccccAAGcuuGAAuuuucAdTsdT-3′ | 5′-AGAAAAUUcAAGCUUGGGGdTsdT-3′ |
| CLEC7A | XD-10564 | <0.004 | 5′-ggGuAAGccAuAAgcGaaudTsdT-3′ | 5′-AUUCGCUuAUGGCUuACCCdTsdT-3′ |
| CLEC7A | XD-10565 | <0.004 | 5′-ccAuAAGcGAAucuuAAuudTsdT-3′ | 5′-AAUuAAGAUUCGCUuAUGGdTsdT-3′ |
| CLEC7A | XD-10566 | 0.006 | 5′-ugccAuAucucuAauAGAAdTsdT-3′ | 5′-UUCuAUuAGAGAuAUGGcAdTsdT-3′ |
| CLEC7A | XD-10567 | 0.023 | 5′-gccuAGAAucuuGuAuAAudTsdT-3′ | 5′-AUuAuAcAAGAUUCuAGGCdTsdT-3′ |
| CLEC7A | XD-10568 | 0.016 | 5′-ccuAGAAucuuGuauAAuAdTsdT-3′ | 5′-uAUuAuAcAAGAUUCuAGGdTsdT-3′ |
| CLEC7A | XD-10569 | 0.016 | 5′-gcucucAuAGGAAaGuuuudTsdT-3′ | 5′-AAAACUUUCCuAUGAGAGCdTsdT-3′ |
| CXorf21 | XD-10502 | 6.027 | 5′-cuGucAGAAGGGuaucucAdTsdT-3′ | 5′-UGAGAuACCCUUCUGAcAGdTsdT-3′ |
| CXorf21 | XD-10503 | 0.654 | 5′-cuAcGuGAGcuGcaAAucAdTsdT-3′ | 5′-UGAUUUGcAGCUcACGuAGdTsdT-3′ |
| CXorf21 | XD-10504 | 1.756 | 5′-gguGAuGGccAuuaAuucAdTsdT-3′ | 5′-UGAAUuAAUGGCcAUcACCdTsdT-3′ |
| CXorf21 | XD-10505 | 13.103 | 5′-cauuAuGGAcAccguGuuudTsdT-3′ | 5′-AAAcACGGUGUCcAuAAUGdTsdT-3′ |
| CXorf21 | XD-10506 | 0.750 | 5′-gucuGAAcucAucauGAcAdTsdT-3′ | 5′-UGUcAUGAUGAGUUcAGACdTsdT-3′ |
| CXorf21 | XD-10507 | n.d. | 5′-gcAAuuGAuGucAacuGAAdTsdT-3′ | 5′-UUcAGUUGAcAUcAAUUGCdTsdT-3′ |
| CXorf21 | XD-10508 | 1.653 | 5′-caAuGuAAAuccAuAGAGAdTsdT-3′ | 5′-UCUCuAUGGAUUuAcAUUGdTsdT-3′ |
| CXorf21 | XD-10509 | n.d. | 5′-guAAGAGGAGccAuAAGGAdTsdT-3′ | 5′-UCCUuAUGGCUCCUCUuACdTsdT-3′ |
| TBXAS1 | XD-10490 | 0.573 | 5′-gccGAcAGcGuucuGuuuudTsdT-3′ | 5′-AAAAcAGAACGCUGUCGGCdTsdT-3′ |
| TBXAS1 | XD-10491 | 0.048 | 5′-cuGcAAGcGuuucuucGAAdTsdT-3′ | 5′-UUCGAAGAAACGCUUGcAGdTsdT-3′ |
| TBXAS1 | XD-10492 | 1.437 | 5′-gcccGGAuuuuGcccAAuAdTsdT-3′ | 5′-uAUUGGGcAAAAUCCGGGCdTsdT-3′ |
| TBXAS1 | XD-10493 | 0.239 | 5′-ggAuuuuGcccAAuAAGAAdTsdT-3′ | 5′-UUCUuAUUGGGcAAAAUCCdTsdT-3′ |
| TBXAS1 | XD-10494 | 0.049 | 5′-caAuAAGAAccGAgAcGAAdTsdT-3′ | 5′-UUCGUCUCGGUUCUuAUUGdTsdT-3′ |
| TBXAS1 | XD-10495 | 0.125 | 5′-gaAccGAGAcGAAcuGAAudTsdT-3′ | 5′-AUUcAGUUCGUCUCGGUUCdTsdT-3′ |
| TBXAS1 | XD-10496 | 0.061 | 5′-agAcGAAcuGAAugGcuuudTsdT-3′ | 5′-AAAGCcAUUcAGUUCGUCUdTsdT-3′ |
| TBXAS1 | XD-10497 | 0.137 | 5′-ccAuGGGcGuGcAaGAcuudTsdT-3′ | 5′-AAGUCUUGcACGCCcAUGGdTsdT-3′ |
| TBXAS1 | XD-10498 | 0.273 | 5′-gcGuGcAAGAcuuuGAcAudTsdT-3′ | 5′-AUGUcAAAGUCUUGcACGCdTsdT-3′ |
| SIGLEC7 | XD-10600 | 0.155 | 5′-agAGuAAccGGAAgCAuusa-3′ | 5′-dTAAUCCUUCCGGUUACUCUusu-3′ |
| SIGLEC7 | XD-10601 | 0.127 | 5′-gaGuAACcGGAAGgAuuasa-3′ | 5′-dTUAAUCCUUCCGGUuACUCusu-3′ |
| SIGLEC7 | XD-10602 | 3.534 | 5′-guAAccGGAAGGAuuAcusa-3′ | 5′-dTAGuAAUCCUUCCGGUuACusu-3′ |
| SIGLEC7 | XD-10603 | 0.029 | 5′-cgGAAGGAuuAcucGcugsa-3′ | 5′-dTCAGCGAGuAAUCCUUCCGusu-3′ |
| SIGLEC7 | XD-10604 | 0.032 | 5′-ggAAGGAuuAcucgcuGasa-3′ | 5′-dTUcAGCGAGuAAUCCUUCCusu-3′ |
| SIGLEC7 | XD-10605 | 0.022 | 5′-ggAuuACucGcuGacGAusa-3′ | 5′-dTAUCGUcAGCGAGUAAUCCusu-3′ |
| SIGLEC7 | XD-10606 | 0.045 | 5′-ccGuGcAAGAGGGcAuGusa-3′ | 5′-dTAcAUGCCCUCUUGcACGGusu-3′ |
| SIGLEC7 | XD-10607 | 0.039 | 5′-cgGGcAGGGAAuGauAuasa-3′ | 5′-dTUAuAUcAUUCCCUGCCCGusu-3′ |
| SIGLEC7 | XD-10608 | 0.056 | 5′-gcAGGGAAuGAuAuAAGcsa-3′ | 5′-dTGCUuAuAUcAUUCCCUGCusu-3′ |
| SIGLEC7 | XD-10609 | 0.059 | 5′-ggAAuGAuAuAAGcuGGasa-3′ | 5′-dTUCcAGCUuAuAUCAUUCCusu-3′ |
| CCR5 | XD-10550 | 0.017 | 5′-ggAAcAAGAuGGAuuAucAdTsdT-3′ | 5′-UGAuAAUCcAUCUUGUUCCdTsdT-3′ |
| CCR5 | XD-10551 | 0.046 | 5′-ccAuAcAGucAGuaucAAudTsdT-3′ | 5′-AUUGAuACUGACUGuAUGGdTsdT-3′ |
| CCR5 | XD-10552 | 0.070 | 5′-cauuAAAGAuAGucAucuudTsdT-3′ | 5′-AAGAUGACuAUCUUuAAUGdTsdT-3′ |
| CCR5 | XD-10553 | 0.361 | 5′-agGuAuGGuuAAuaAGuuudTsdT-3′ | 5′-AAACUuAUuAACcAuACCUdTsdT-3′ |
| CCR5 | XD-10554 | n.d. | 5′-gauccuGGuuGGuguuGcAdTsdT-3′ | 5′-UGcAAcACcAACcAGGAUCdTsdT-3′ |
| CCR5 | XD-10555 | n.d. | 5′-guAuGAGGucuAGgAAcAUdTsdT-3′ | 5′-AUGUUCCuAGACCUcAuACdTsdT-3′ |
| CCR5 | XD-10556 | n.d. | 5′-caucAAAcucuuAguuAcudTsdT-3′ | 5′-AGuAACuAAGAGUUUGAUGdTsdT-3′ |
| CCR5 | XD-10557 | n.d. | 5′-cuccGuAuuucAGacuGAAdTsdT-3′ | 5′-UUcAGUCUGAAAuACGGAGdTsdT-3′ |
| CCR5 | XD-10558 | 1.052 | 5′-gcAcAuAcuuGAGacuGuudTsdT-3′ | 5′-AAcAGUCUcAAGuAUGUGCdTsdT-3′ |
| CCR5 | XD-10559 | 1.018 | 5′-gcAAcGAAGGGAAauGucudTsdT-3′ | 5′-AGAcAUUUCCCUUCGUUGCdTsdT-3′ |
| DOCK2 | XD-09690 | n.d. | 5′-cacGGcGuGGccAuAuAcAdTsdT-3′ | 5′-UGuAuAUGGCcACGCCGUGdTsdT-3′ |
| DOCK2 | XD-09691 | 11.713 | 5′-ggGGAuAccucAuaAAGcAdTsdT-3′ | 5′-UGCUUuAUGAGGuAUCCCCdTsdT-3′ |
| DOCK2 | XD-09692 | n.d. | 5′-caAGcAAAcGGucauAAGudTsdT-3′ | 5′-ACUuAUGACCGUUUGCUUGdTsdT-3′ |
| DOCK2 | XD-09693 | n.d. | 5′-uuGuGuAcuAucAaGucAAdTsdT-3′ | 5′-UUGACUUGAuAGuAcAcAAdTsdT-3′ |
| DOCK2 | XD-09694 | 0.403 | 5′-ccAucuGcGAuucauGuuudTsdT-3′ | 5′-AAAcAUGAAUCGcAGAUGGdTsdT-3′ |
| DOCK2 | XD-09695 | n.d. | 5′-cucuAcAcGAuGGauuccAdTsdT-3′ | 5′-UGGAAUCcAUCGUGuAGAGdTsdT-3′ |
| DOCK2 | XD-09696 | 1.775 | 5′-cucAuuGcAGAccgGAAAudTsdT-3′ | 5′-AUUUCCGGUCUGcAAUGAGdTsdT-3′ |
| DOCK2 | XD-09697 | 0.556 | 5′-accGGAAAuuucAgcAuuudTsdT-3′ | 5′-AAAUGCUGAAAUUUCCGGUdTsdT-3′ |
| DOCK2 | XD-09698 | 1.356 | 5′-ggucGAGGAcAuuauuuucdTsdT-3′ | 5′-GAAAAuAAUGUCCUCGACCdTsdT-3′ |
| FLuc | XD-00194 | n.d. | 5′-cuuAcGcuGAGuAcuucGAdTsdT-3′ | 5′-UCGAAGuACUcAGCGuAAGdTsdT-3′ |
| (control) | | | | |

The validated siRNAs were then used in primary human macrophage assays to determine the ability of target knockdown to alter the pro-tumorigenic (M2) or pro-inflammatory (M1) phenotype as described above in Example 1. Briefly, monocytes were isolated from whole blood of fresh donors by Ficoll separation with RosetteSep™ Human Monocyte Enrichment Cocktail (Stemcell Techhnologies, Vancouver Canada) according to the manufacturer's instructions. Isolated monocytes were arrayed in 24-well plates overnight in Iscove's Modified Dubelcos Media (IMDM) (ThermoFisher) media containing 10% fetal bovine serum and non-adherent cells were washed off after 24 hours. Monocytes were differentiated into macrophages by culturing for 6 days in MDM 10% FBS plus 50 ng/ml human M-CSF for M2 macrophages, or 50 ng/ml GM-CSF (Biolegend, San Diego, Calif.) for M1 macrophages.

siRNA lipid nanoparticles were administered at a final concentration of 50 nM on Day 1 and Day 3. C12-200 lipid nanoparticles (LNPs) were formulated as described in Novobrantseva el al. (2012) Mol Ther. Nucl. Acids 1:e4. Briefly, an ethanolic phase containing the ionizable lipid C12-200 (described in Love et al. (2010) AXO Labs GmbH, Kulmbach, Germany; available on the World Wide Web at doi.org/10.1073/pnas.0910603106), distearoyl-sn-glycero-3-phosphocholine (DSPC, Avanti Polar Lipids, Alabaster Ala.), cholesterol (MP Biomedicals, Santa Ana Calif.), and DMPE-PEG2000 (1,2-dimyristoyl-sn-glycero-3-phosphoethanolamine-N-[methoxy(polyethylene glycol)-2000], Avanti) at a molar ratio of 50:10:38.5:1.5 were mixed together with an aqueous phase of siRNA in 10 mM citrate buffer via microfluidic mixing. The ethanol:aqueous volume ratio was 1:3, and the total lipid:siRNA weight ratio was approximately 9:1. The resulting LNPs were dialyzed against 1×PBS overnight. Formulated LNPs had median particle diameters of approximately 60-70 nm as measured by nanoparticle tracking analysis (ZetaView, ParticleMetrix) and siRNA encapsulation efficiencies of approximately 80-90% as measured by a modified Quant-iT™ RiboGreen® assay (Heyes et al., 2005; doi:10.1016/j.jconrel.2005.06.014).

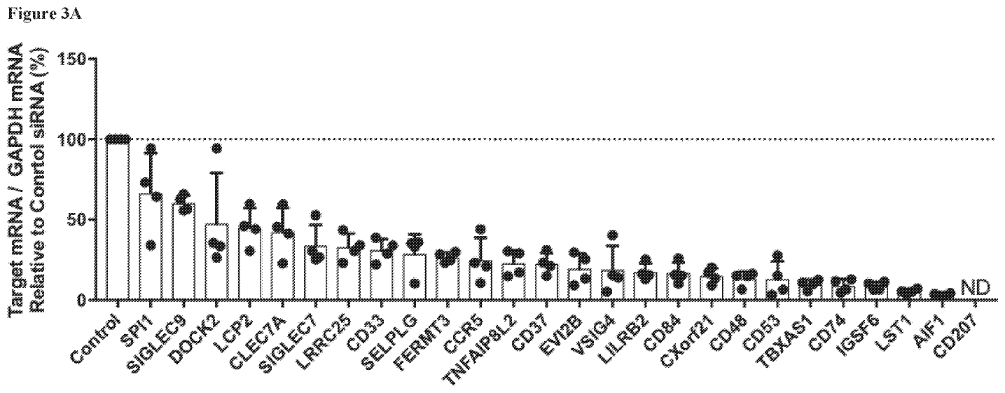

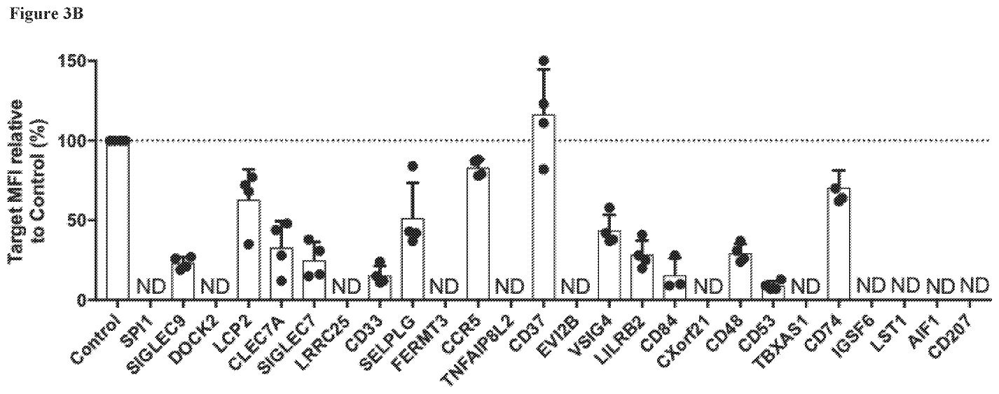

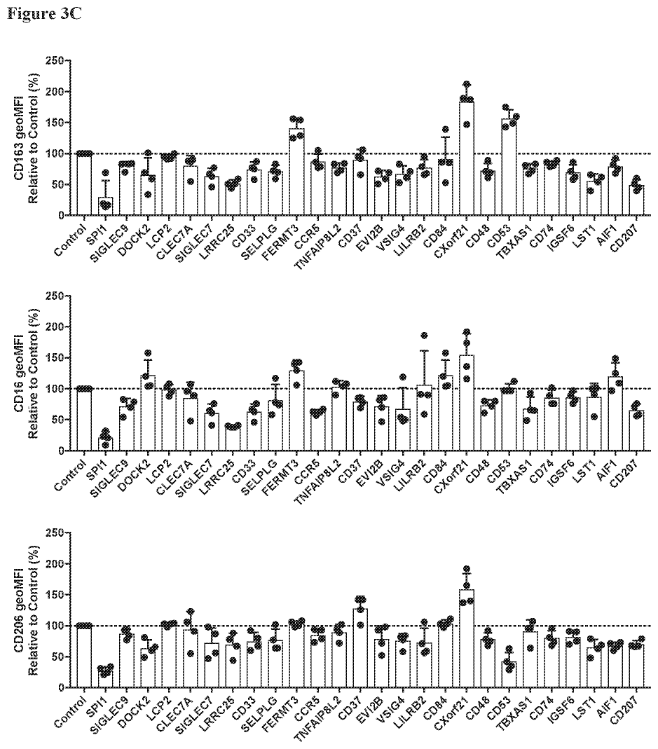

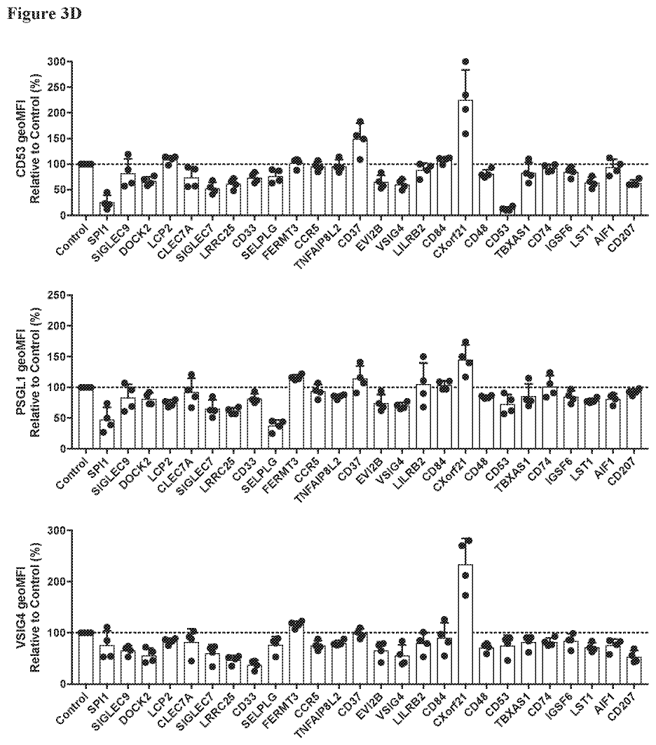

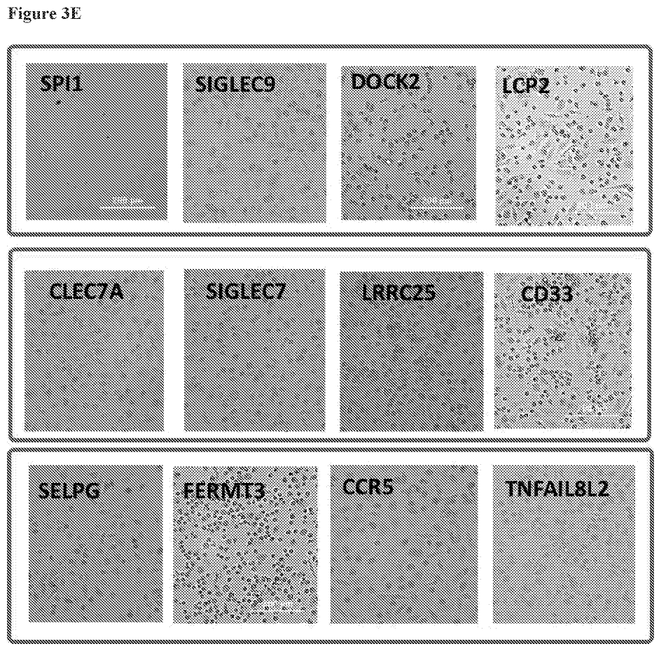

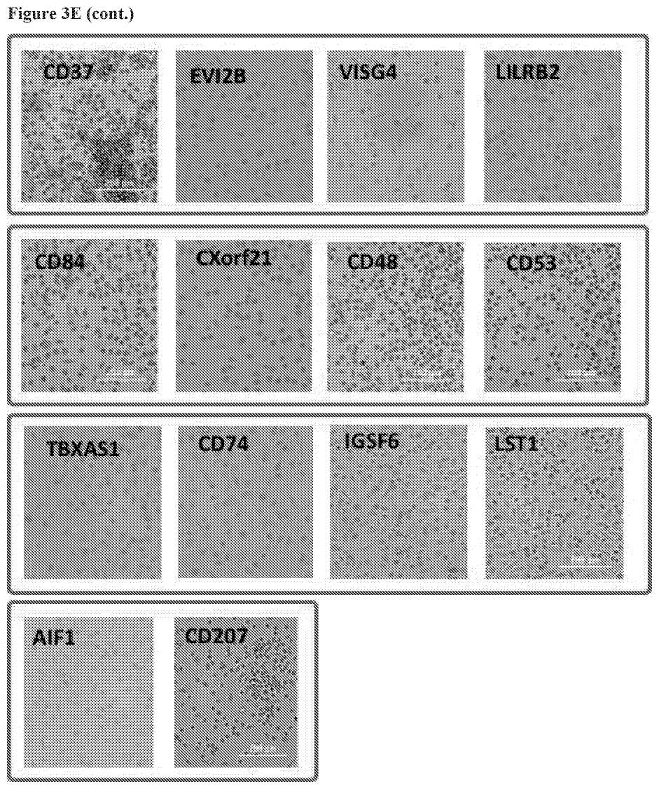

On day 6 of culture M2 macrophages were polarized with 20 ng/ml human IL-10 (Biolegend, San Diego, Calif.). Forty-eight hours later, macrophages were removed from plates by scraping and mRNA levels were assessed by bDNA as described above. FIG. 3A shows the relative knockdown of each individual target assessed in M2 macrophages. FIG. 3B shows the knockdown of the target protein if they are surface expressed, in the same M2 macrophages, via flow cytometric anlaysis.

FIG. 3 shows the results from the siRNA-treated M2 macropahges described above demonstrating 26 validated targets that were determined to drive M2 macrophages toward an M1 phenotype and/or further along the M2 spectrum, such as by assessment using the expression of traditional markers of CD163, CD16, and CD206, as well as via biomarkers described in Example 1 above, such as CD53, PSGL1, and VSIG4. Additionally, these targets demonstrated an ability to alter the morphology of these M2 macrophages to be M1-like and/or more M2-like. Importantly, cells within these assays undergoing differentiation remain in the presence of the skewing conditions through the entirety of the assay. Thus, for an siRNA to drive a target from M2-like to M1-like, it must do so in the presence of a continuous strong skewing cocktail. This sets a very high bar for function of these siRNAs as they do not achieve full knockdown, yet significant phenotypic changes are demonstrated. Example 1 demonstrates the expression of traditional macrophage markers CD163, CD16 and CD206, as well biomarkers CD53, PSGL1, and VSIG4. FIG. 1 also shows the natural and significant variability within these markers between individual donors. Also, of note is the difference in expression between M1 differentiated cells and M2 differentiated cells for some markers is less than a 0.5 fold change (FIG. 1A), indicating that even small expression changes can have very dramatic functional consequences. Therefore, targets from Table 1 and Table 2 were considered validated, via siRNA knockdown, when the mean change of a minimum of 4 donors was 10% or greater of either; i) a classic marker, ii) a new biomarker, iii) a combination of classic or new biomarkers, or iv) i, ii, or iii in combination with a morphologic change.

### Example 3: Validation of Targets that Modulate Macrophage Inflammatory Phenotype by Blocking Target Protein

Macrophages are biologically optimized to either induce or suppress an immune response. Therefore, targeting macrophages via siRNA, antibody, or other modality allows for the alteration of the initiation, suppression and/or perpetuation of immune responses.

Antibodies to targets validated as described in Example 2 have been identified and generated. Antibodies used in experiments described herein were sourced from commercial vendors or generated recombinantly. For example, antibody variable region sequences were derived from known binders, such as those described in the following sources: U.S. Pat. Publ. No. 2007/0160601, U.S. Pat. Nos. 7,833,530, 7,604,802, and U.S. Pat. Publ. No. 2017/0190782. Antibody variable region sequences are described in Table 6 or produced.

TABLE 6
Antibody variable region sequences

| Antibody | Antibody | |
|---|---|---|
| Name | Region | Sequence |
| AB 5 | VH | EVQLQQSGPDLVKPGALVKISCKASGYSFTAYYIHWVKQSHGKSLEWIGRVNPNTGGTSYN |
| | | PKFKGKAILNVDKSSSTAYMELRSLTSEDSAVYYCARSGSPYYRYDDWGQGTTLTVSS |
| AB 5 | VL | ENVLTQSPAIMSASPGEKVTMTCRASSTVNSTYLHWFQQKSGASPKLWIYGSSNLASGVPA |
| | (kappa) | RFSGSGSGTSYSLTISSVEAEDAATYYCQQYSGYPLTFGAGTTLELK |
| AB 6 | VH | EVQLVETGGGLVQPKGSLKLSCAASGFTFNTNAMNWVRQAPGKGLEWVARIRSKSNNYATY |
| | | YASVKDRFTISRDDTQSMIYLQMNNLKTEDTGMYYCVRGGSYWYFDVWGAGTTVTVSS |
| AB 6 | VL | DVLMTQTPLSLPVSLGDQASISCRSSQSIVNSNGNTYLEWYLQKPGQSPKLLIYKVSNRFS |
| | (kappa) | GVPDRFSGSGSGTDFTLKISRVEAEDLGVYYCFQGSHVPWTFGGGTKLEIK |
| AB 7 | VH | EVQLVESGGGLVQPGGSLRLSCAASGFTFSSFGMHWVRQAPGKGLEWVAYINGGSSTIFYA |
| | | NAVKGRFTISRDNAKNTLYLQMNSLRAEDTAVYYCARASYGGGAMDYWGQGTLVTVSS |
| AB 7 | VL | DIQMTQSPSSLSASBGDRVTITCRSSQSIVHNDGNTYFEWYQQKPGKAPKLLIYKVSNRFS |
| | (kappa) | GVPSRFSGSGSGTHFTLTISSLQPEDFATYYCFQGSYVPLTFGQGTKVEIK |
| AB 8 | VH | QVQLQQSGPGLVKPSQTLSLTCAISGDSVSSNIAAWHWIRLSPSRGLEWLGRTYYRRSKWN |
| | | YDYALSVKSRININPDTSKNLFSLQLNSVTPEDTAVYYCTRGGGRAHSAWGQGTLVTVSS |
| AB 8 | VL | EIVLTQSPGTLSVSPGERATLSCRASQSVSRSHLAWYQQKPGQAPRLLIFGASSRATGIPD |
| | (kappa) | RFSGSGSGTDFTLTISRLEPEDFAVYYCQQYGRPGVTFGQGTKVEIK |
| AB 16 | VH | QVQLVQSGAEVKKPGSSVRVSCKASGGSFSSYGIHWVQQAPGQGLEWMGRIIPVLRIRNYA |
| | | QKFHDRVTIDADTSTGTAYMELSSLTSDDTAVYYCAGSRQGVAPTGYWGQGTMVTVSS |
| AB 16 | VL | QAVLTQPSSLSASPGASASLTCNLRSGIDVGTYRIYWYQQKPGSPPQYLLRYKSDSDKQQG |
| | (lambda) | SGVPSRFSGTKDASANAGILLISGLQSEDEADYYCMIWHSNGWVFGGGTKLTVL |
| AB 18 | VH | EVQLLESGGGLVRPGGSLRLSCAASGFTFSSYAMSWVRQAPGKGLEWVSAISGSGGSTYYA |
| | | DSVKGRFTISRDNSKNTLYLQMNSLRAEDTAVYYCARDKRLTYWGQGTMVTVSS |
| AB 18 | VL | QSVVTQPPSVSAAPGQKVTISCSGSSSNIGNNYVSWYQQLPGTAPKLLIYDNNKRPSGIPD |
| | (lambda) | RFSGSKSGTSATLGITGLQTGDEADYYCGTWDSSLSARVFGGGTKVTVL |
| AB 33 | VH | EVQLQQSGPELVKPGASVKMSCKASGYTFTSYVMHWMKQKPGQGLEWIGYINPYNDGTKYN |
| | | DKFKGKATLTSDKSSSTAYMELSSLTSEDSAVYYCARGSNYEDFAMDYRGQGTSVTVSS |
| AB 33 | VL | DIVMTQSPASQSASLGESVTITCLASQTIGTWLAWYQQKPGKSPQLLIYAATTLADGVPSR |
| | (kappa) | FSGSGSGTKFSFKISSLQAEDFVSYYCQQLYSTPLTFGGGTKVEIK |
| AB 34 | VH | EVQLQQSGPELVKPGASVKVSCKASGYAFTSYNMYWVKQSHGKSLEWIGYIDPYNGGTRHN |
| | | QKFKDKATLTVDKSSSTAYMHLNSLTSEDSAVYYCASQNYEYFDYWGQGTTLTVSS |
| AB 34 | VL | DIVLTQSPKFMSTSVGDRVSITCKASQDVGDAVAWYQQKPGQSPKLLFYWTSTRHTGVPDR |
| | (kappa) | FTGSGSGTEFTLTIRNVQSEDLADYFCQQYRSTPLTFGSGTKVEIK |

All recombinant antibodies were expressed as human IgG4 chimeras with a S228P heavy chain mutation paired with either kappa or lambda light chain. Variable heavy chain (HC) and light chain (LC) sequences were cloned into vectors containing the antibody constant region sequences shown in Table 7.

TABLE 7
Antibody constant region sequences

| Region | Sequence |
|---|---|
| hIgG4 | ASTKGPSVFPLAPCSRSTSESTAALGCLVKDYFPEPVTVSWNSGALTSGVHTFPAVLQSSGLYSLSSVVTVPSS |
| (S228P) | SLGTKTYTCNVDHKPSNTKVDKRVESKYGPPCPPCPAPEFLGGPSVFLFPPKPKDTLMISRTPEVTCVVVDVSQ |
| | EDPEVQFNWYDGVEVHNAKIKPREEQFNSTYRVVSVLTVLHQDWLNGKEYKCKNVSNKGLPSSIEKTISKAKGQ |
| | PREPQVYTLPFSQEEMTKNQVSLTCLVKGFYPSDIAVEWESNGQPENNYKTTPPVLDSDGSFFLYSRLTVDKSR |
| | WQEGNVFSCSVMHEALHNHYTQKSLSLSLGK |
| hKappa LC | RTVAAPSVFIFPPSDEQLKSGTASVVCLLNNFYPREAKVQWKVDNALQSGNSQESVTEQDSKDSTYSLSSTLTL |
| | SKADYEKHKVYACEVTHQGLSSPVTKSFNRGEC |
| hLambda LC | GQPKAAPSVTLFPPSSEELQANKATLVCLISDFYPGAVTVAWKADSSPVKAGVETTTPSKQSNNKYAASSYLSL |
| | TPEQWKSHRSYSCQVTHEGSTVEKTVAPTECS |

Protein expression and purification was performed by ATUM (Newark, Calif.), by transient transfection of heavy chain- and light chain-containing proprietary vectors into suspension-adapted HEK293 cells. Cell culture supernatant was purified by protein A affinity chromatography (MabSelect SuRe™ pcc, GE Life Sciences) according to the manufacturer's protocol. Eluted, neutralized proteins were buffer-exchanged into PBS, pH 7.4 (Corning) and filter-sterilized. Purified antibodies were quantified by OD280 using extinction coefficients calculated from the primary amino acid sequence. Purified antibodies were characterized by capillary gel electrophoresis (Perkin Elmer GXII) or SDS-PAGE (Bio-Rad Criterion™ Tris/Glycine/SDS, 4-20%) and HPLC-SEC. Endotoxin levels were also characterized (Charles River Endosafe™).

Commercial antibodies listed in Table 8 were purchased and used in the assays. In addition, antibodies listed in Table 9 were generated against validated targets described in Example 2 and included in these assays.

TABLE 8
Commercial antibodies

| Antibody Name | Vendor | Clone Name | Antigen |
|---|---|---|---|
| AB 62 | RND Systems | 287219 | LILRB2 |
| AB 63 | BioLegend | WM53 | CD33 |
| AB 64 | BioLegend | KPL-1 | PSGL1 |
| AB 65 | Ebiosciences | 156-4H9 | CD48 |
| AB 66 | NovusBio | 394607 | CD48 |
| AB 67 | RND Systems | 424925 | CD37 |
| AB 68 | Ebiosciences | GE2 | Dectin |
| AB 69 | Merck | Keytruda | PD1 |
| AB 70 | BioLegend | QA16a12 | hIgG1 Isotype |
| AB 71 | BioLegend | QA16A15 | hIgG4 Isotype |
| AB 72 | BioLegend | LN2 | CD74 |
| AB 73 | BioLegend | MOPC-21 | mIgG1 Isotype |
| AB 74 | BioLegend | CD84.1.2 | CD84 |

TABLE 9
ATCC-Deposited Antibodies

| Antibody | ATCC Deposition # | Antigen |
|---|---|---|
| AB 75 | 13H10 | Siglec9 |
| | (PTA-125944) | |
| | (Ike9.m.13H10.hyb.mG1) | |
| AB 76 | 13J19 | Siglec9 |
| | (PTA-125945) | |
| | (Ike9.m.13J19.hyb.mG1) | |
| AB 77 | 18F02 | PSGL1 |
| | (PTA-125946) | |
| | (Hn1.m.18F02.hyb.mG1) | |
| AB 78 | 19I01 | PSGL1 |
| | (PTA-125943) | |
| | (Hn1.m.19I01.hyb.mG1) | |
| AB 79 | 3C01 | LRC25 |
| | (PTA-126026) | |
| | (Lf2.m.FJP5_3C01.hG4) | |
| AB 80 | 1F01 | LRC25 |
| | (PTA-126025) | |
| | (Lf2.m.FJP5_1F01.hG4) | |
| AB 81 | 7A12 | CD53 |
| | (PTA-126029) | |
| | (Px1.m.7A12.hyb.mG2b) | |
| AB 82 | 7F19 | CD53 |
| | (PTA-126030) | |
| | (Px1.m.7F19.hyb.mG2b) | |
| AB 83 | 10M15 | CD53 |
| | (PTA-126027) | |
| | Px1.m.10M15.hyb.mG3 | |
| AB 84 | 4H23 | CD53 |
| | (pTA-126028) | |
| | (Px1.m.4H23.hyb.mG2b) | |

The generated antibodies to targets validated as described in Example 2 have been utilized in functional assays. The effect of these antibodies on macrophage differentiation state was measured by readouts, including macrophage state-specific biomarkers, cytokine secretion, and other functional characteristics, such as the ability to perpetuate a concerted immune response in complex multi-cellular assays.

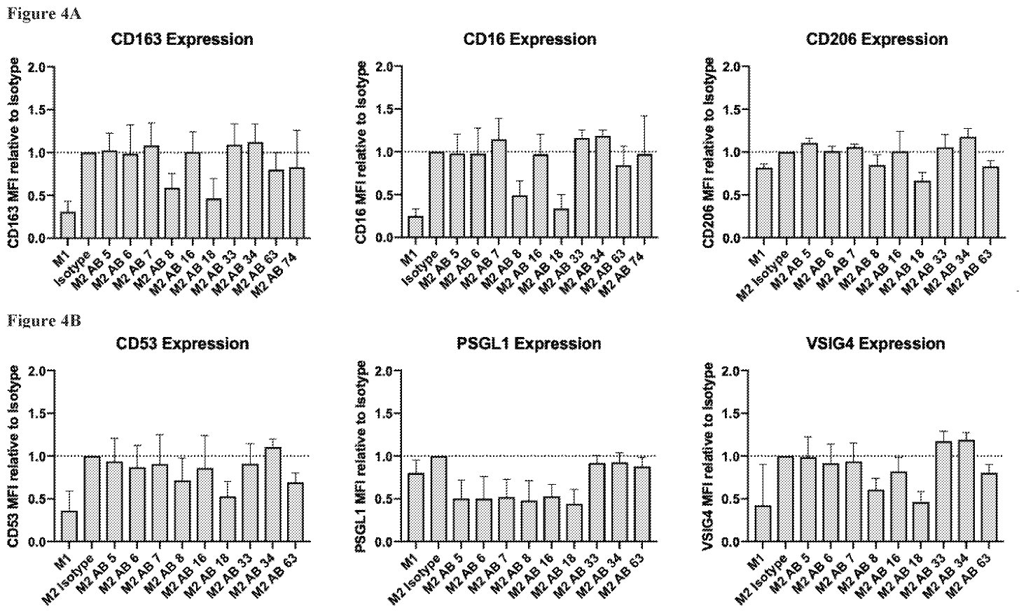

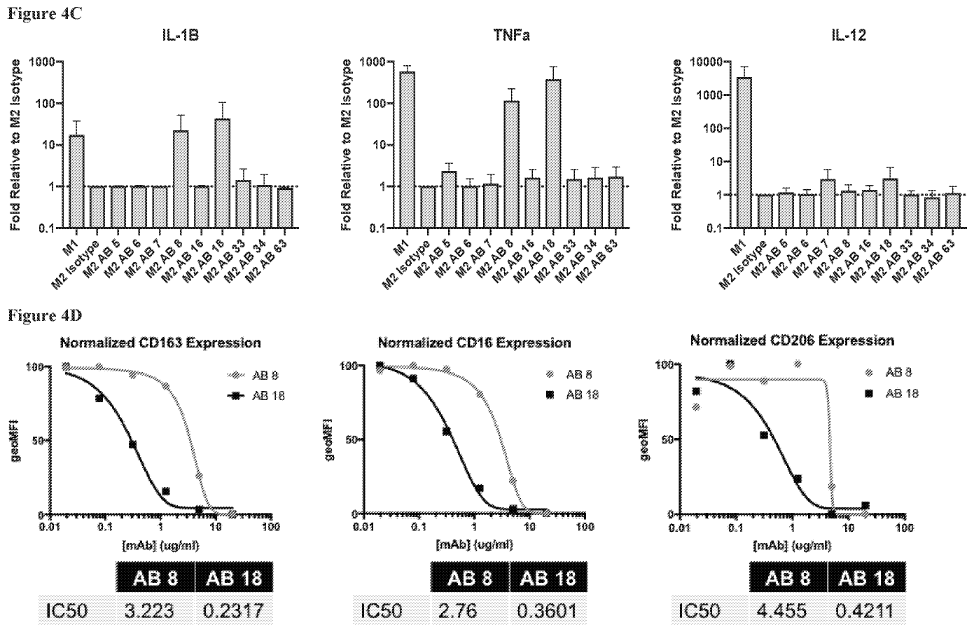

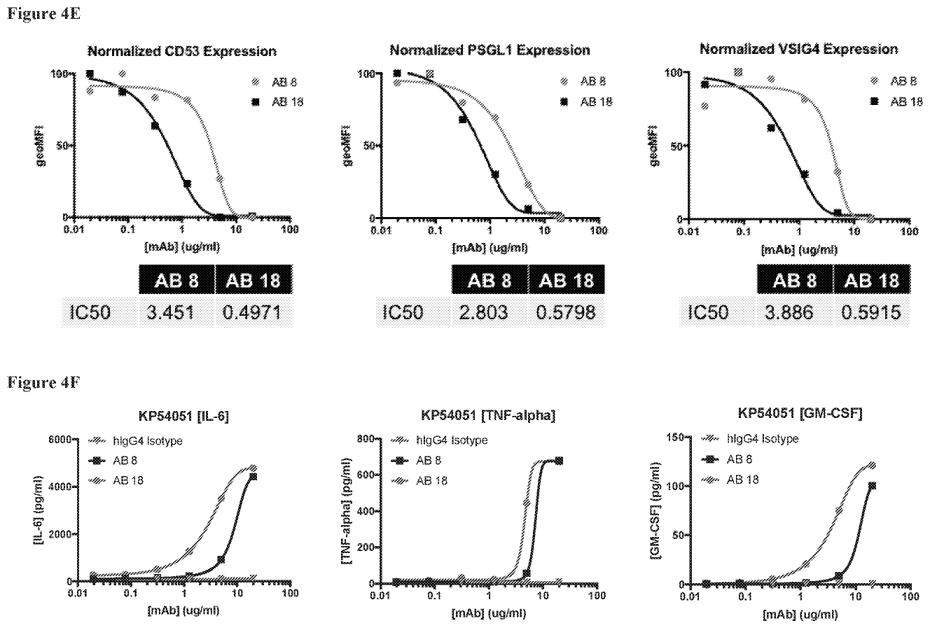

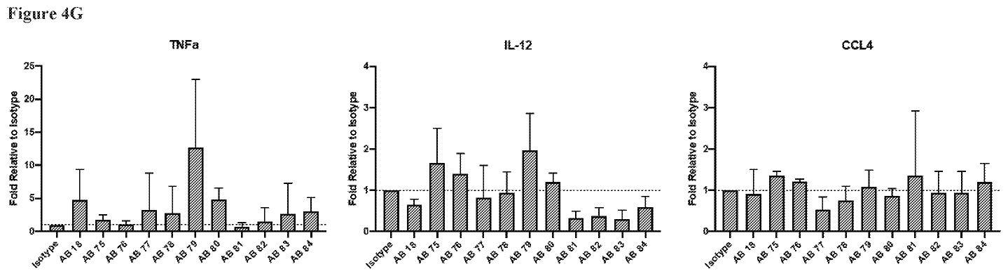

For example, FIG. 4 shows the results of 20 antibodies listed in Tables 6,8, and 9 that were utilized in macrophage differentiation assays.

Briefly, monocytes were isolated from whole blood of fresh donors by Ficoll separation with RosetteSep™ Human Monocyte Enrichment Cocktail (Stemcell Techhnologies, Vancouver Canada) according to the manufacturer's instructions. Isolated monocytes were arrayed in 24-well plates overnight in Iscove's Modified Dubelcos Media (EWDM) (ThermoFisher) media containing 10% fetal bovine serum and non-adherent cells were washed off after 24 hours. Monocytes were differentiated into macrophages by culturing for 6 days in IMDM 10% FBS plus 50 ng/ml human M-CSF for M2 macrophages, or 50 ng/ml GM-CSF (Biolegend, San Diego, Calif.) for M1 macrophages. M1 macrophages were activated on day 6 with IFN-gamma and LPS, whereas M2 macrophages were polarized on day 6 with 20 ng/ml IL-10 and activated on day 7 with 100 ng/ml LPS. Monoclonal antibodies listed in Tables 6 and 8 were administered at a final concentration of 10 ug/ml on day 1, 3 and 7 of culture. Cells were assessed for expression of M1 and M2 markers as described above and cell free supernatants were collected for analysis. Data are representative of at least 3-4 healthy donors.

Specific antibodies were able to reverse the skewing towards the M2 phenotype, as determined by both classical and novel biomarkers described herein, such as in Example 1. This is not a pan-functional effect for all antibodies against a target since not all mAbs directed against a given target induced a notable change in macrophage phenotypic markers. Furthermore, antibodies Ab 8 and Ab 18 were able to demonstrate a dose-titratable effect on all of these marker sets (FIG. 4). As above, targets were considered validated, via antibody treatment, when the mean change between a minimum of 4 donors was 10% or greater of either; i) a classic marker, ii) a new biomarker, iii) a combination of classic or new biomarkers, or iv) i, ii, or iii in combination with a morphologic change.

Beyond phenotypic surface markers, M1 (such as Type 1) and M2 (such as Type 2) macrophages produce different cytokines and chemokines. For example, M1 macrophages produce more pro-inflammatory cytokines, including but not limited to, GM-CSF, IL-12, and TNF alpha, whereas M2 macrophages produce more pro-tumorigenic and immunosuppressive cytokines, such as VEGF, IL-10, and TGFb. This effect can be seen in FIG. 4C where M1-differentiated macrophages produce high levels of pro-inflammatory cytokines as compared to M2 macrophages. Throughout these assays the macrophages are strongly driven, via the presence of potent cytokines IL-10 and M-CSF, to an M2 phenotype. The addition of mAbs that bind to validated targets, throughout the differentiation process, are able to overcome this potent polarization and drive the M2 macrophages to a more M1 like state. This is evidenced by the change in not only classic phenotypic markers but also novel biomarkers, as well as in functional induction of pro-inflammatory cytokine production.

The ability to effect a response when the siRNA or antibody that is added for the entirety of the differentiation and polarization process has been demonstrated above. In a disease setting, such as a tumor, it is believed that the cells will already be differentiated to some extent along the M2 spectrum. Therefore, in FIGS. 4D-4G, monocytes were polarized to M2 as described above, but antibodies were added only during the last two days of the polarization process. Furthermore, antibodies 77, 78, and 81-84 were dosed at 1 ug/mL rather than the 10 ug/mL that all other mAbs were dosed at as noted above. During this limited window, mAbs 8, 18, and 75-82 were able to dramatically effect polarization of M2 macrophages to a more M1-like state as demonstrated by the increase in pro-inflammatory cytokines. As demonstrated in Example 2, down-regulation versus siRNA knockdown of some targets led the macrophages to achieve a more M2-like immunosuppresive phenotype. mAbs 83 and 84 demonstrate the ability to recapitulate this functional effect via target blocking with mAbs.

These figures demonstrate the ability of antibodies to validated targets to reverse both the phenotype, as well as functional characteristics, of M2 macrophages to make them more M1-like, as well as drive macrophages to a more immunosuppresive M2 like phenotype.

### Example 4: Validation of Targets that Modulate Macrophage Inflammatory Phenotype Using Complex Multicellular Assays

In order for macrophages to induce tumor immunogenicity or reverse the course of autoimmune and inflammatory disorders, they generally should able to induce or block a concerted immune response. This would include having direct and downstream effects on both myeloid and lymphoid cells. Complex multi-cellular assays consisting of primary cells from both the lymphoid and myeloid lineage are needed to analyze such effects.

Several systems have been utilized to demonstrate the ability of validated targets described herein to lead to a concerted immune response, including a Staphylococcal enterotoxin B (SEB) assay and a mixed lymphocyte reaction (MLR) assay. These assays take advantage of primary human cells, which are the most natural cells to study and have the best predictive power for in vivo disease, such as human disease. These assays naturally have high variability from donor to donor both in the amplitude of background activity and response.

For the SEB assay, peripheral blood mononuclear cells (PBMCs) were isolated from blood of fresh donors by Ficoll® separation and frozen in 90% fetal bovine serum (FBS), 10% DMSO at −150° C. for long term storage. PBMCs were thawed into complete RPMI media containing 10% FBS, 50 nM 2-mercaptoethanol, non-essential amino acids, 1 mM sodium pyruvate, and 10 mM HEPES. Next, 200,000 cells were plated in each well of a 96-well plate in complete RPMI. Anti-human PD-1 pembrolizumab (Merck, KEYTRUDA®, MK-3475) was added at 5 μg/ml and other antibodies indicated in Tables 6-9 were added at 10 μg/ml as indicated with the exception of mAbs 77, 78, and 81-84, which were added at 1 ug/mL. Cells and mAbs were incubated at 37° C. for 30 minutes and Staphylococcal enterotoxin B (SEB) (EMD Millipore, Billerica, Mass.) was added at a final concentration of 0.1 μg/ml. After 4 days of activation, supernatant was collected and frozen at −20° C. Cytokine concentration was measured using multi-parameter ProcartaPlex™ Assay (ThermoFisher Scientific). Data are representative of at least 4 healthy donors.

For the MLR assay, monocytes and T cells were isolated from MHC-mismatched donors by first isolating PBMCs from whole blood by gradient centrifugation over Ficoll® Paque Plus (GE Healthcare, Chicago Ill.). Monocytes were isolated from PBMC using the EasySep™ Human Monocyte Enrichment Kit without CD16 depletion (StemCell Technologies, Vancouver Canada) according to the manufacturer's manual. When indicated, monocytes were differentiated into M2 macrophages in the presence of antibodies as described above. T cells were isolated from PBMCs using the EasySep™ Human T Cell Isolation Kit (StemCell Technologies, Vancouver Canada) according to the manufacturer's manual. Allogeneic mixed lymphocyte reactions were set up by plating 20,000 monocytes (M0) in U bottom 96-well plates and pre-incubating them with 1 ug/mL of the indicated antibody at 37° C. for 30 min. Then, 50,000 T cells were added to each well and cells were incubated in a humidified incubator at 37° C./5% CO2 for 3 days. Culture media was IMDM+10% FCS (fetal calf serum). On day 3, cells were re-stimulated for 4 h using the T Cell Activation Cocktail with brefeldin A (BioLegend, San Diego, Calif.) according to the manufacturer's manual. Where indicated, supernatant was collected and T cells were analyzed by flow cytometry on an Attune flow cytometer (Thermo Fisher Scientific, Waltham, Mass.) and analyzed using FlowJo Software (BD Bioscience, San Jose, Calif.). Cytokines from supernatant were measured using a Luminex panel (Thermo Fisher, Waltham, Mass.) according to the manufacturer's protocol. Luminescence was detected using a Cytation 5 Imaging Reader (Biotek, Winooski, Vt.). Data were presented as normalized to isotype control groups.

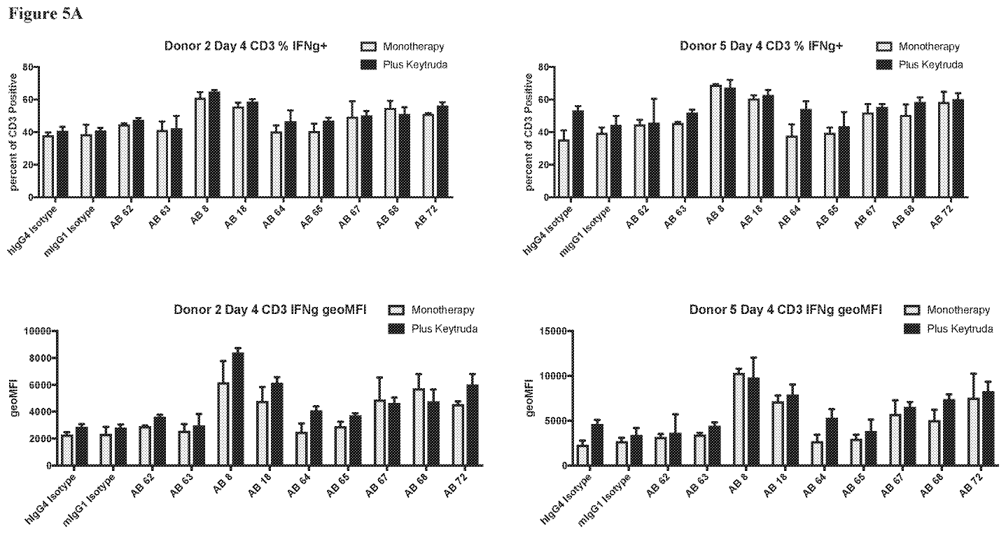

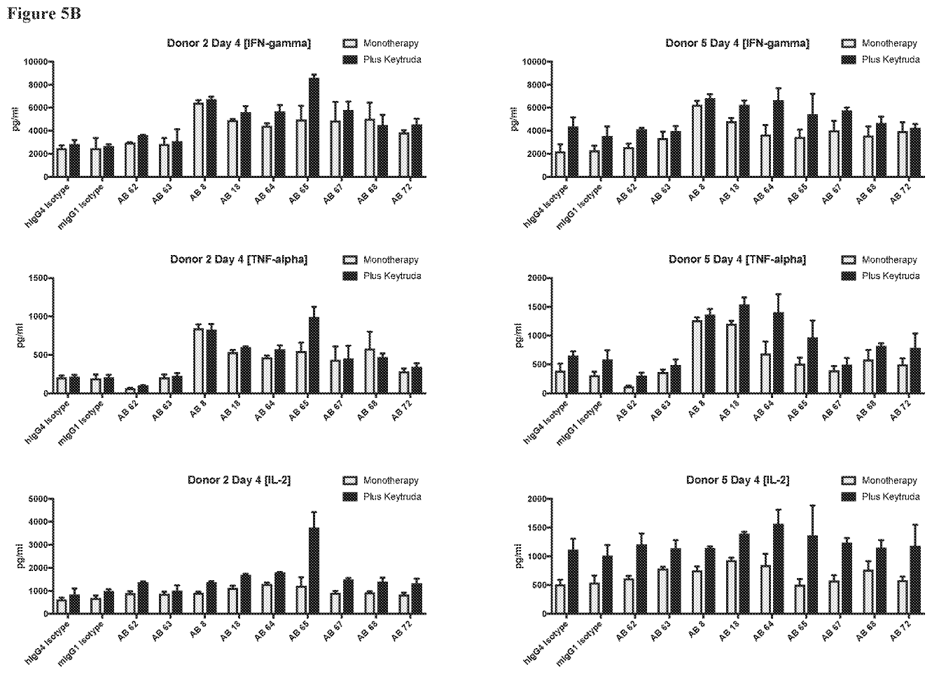

In these assays, specific antibodies to validated targets were demonstrated to be able to impact a concerted multi-cellular immune response. This concerted multi-cellular response included not only altering the phenotype and function of myeloid cells as previously demonstrated but also the functional output of lymphoid cells, specifically T cells. The results of SEB assays are shown in FIG. 5. FIG. 5A demonstrates T cell specific intra-cellular staining of IFNγ. As can be seen specific validated mAbs are able to increase IFNγ above control levels. FIGS. 5B and 5C demonstrate secreted cytokine levels from the SEB assay. Treatment with validated mAbs led to changes in the production of myeloid-derived cytokines and chemokines (e.g., IL-1B, GM-CSF, and CCL3.4) and T cell derived cytokines (e.g., IL-2, IFNγ, and IL-10). This clearly indicates that mAbs that were validated to drive macrophages to a more pro-inflammatory M1-like state can have a consistent effect in multi-cellular assay and increase inflammatory cytokines, as well as mAbs that were validated to drive macrophages to a more immunosuppressive state recapitulated that effect in a complex multi-cellular assay.

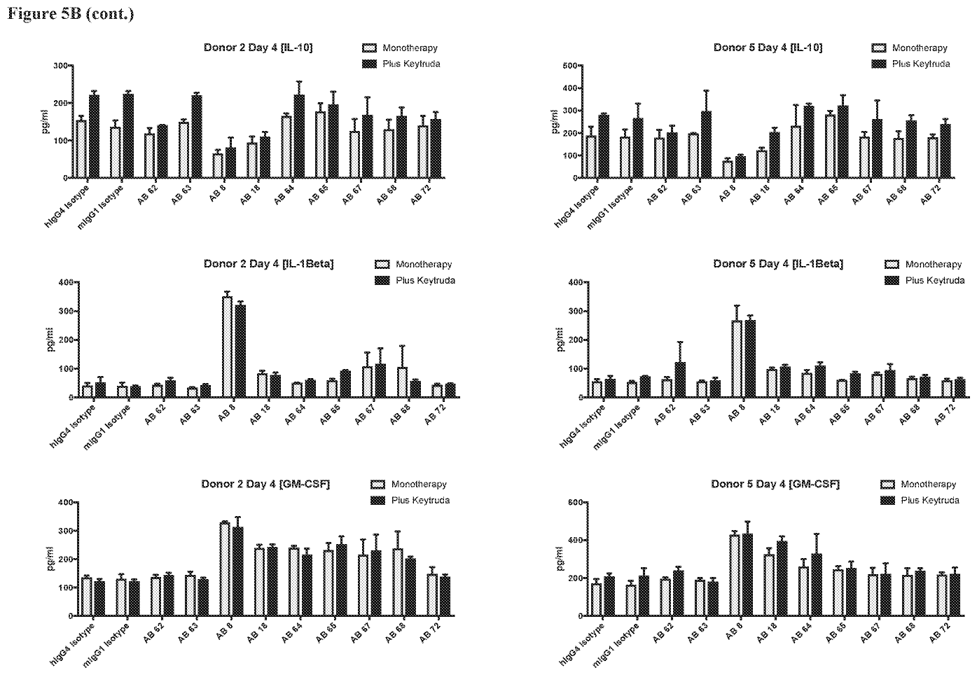

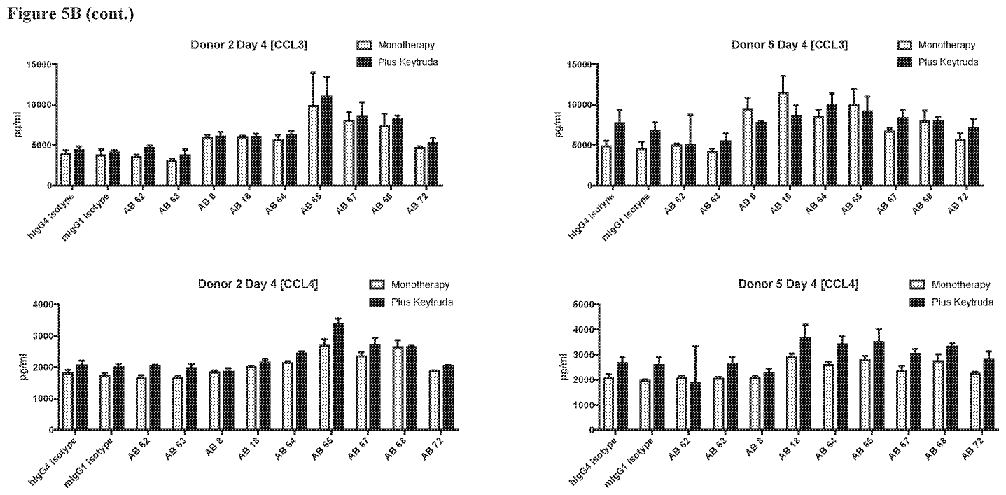

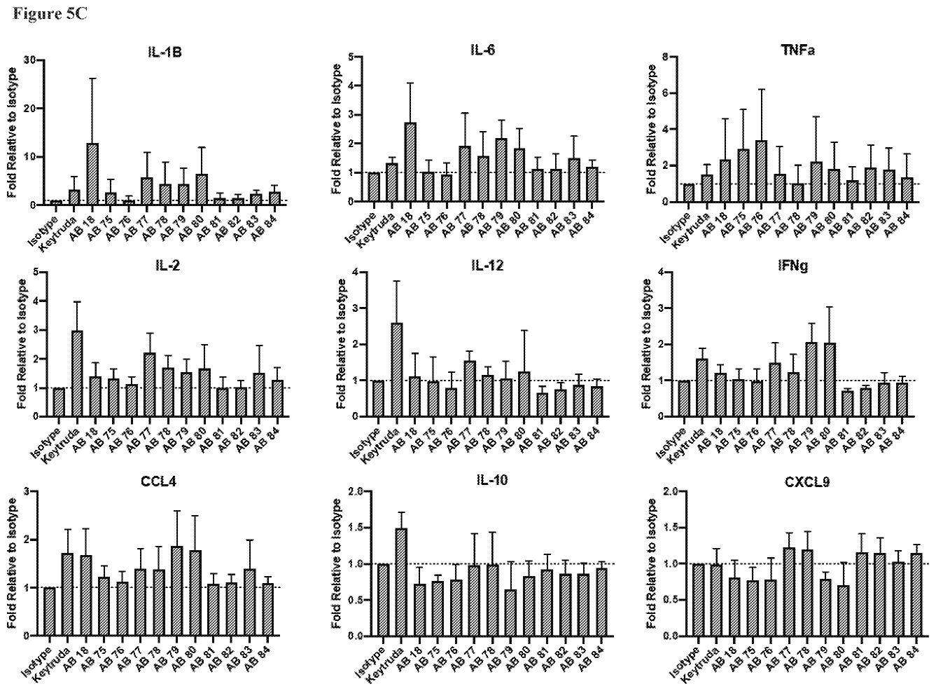

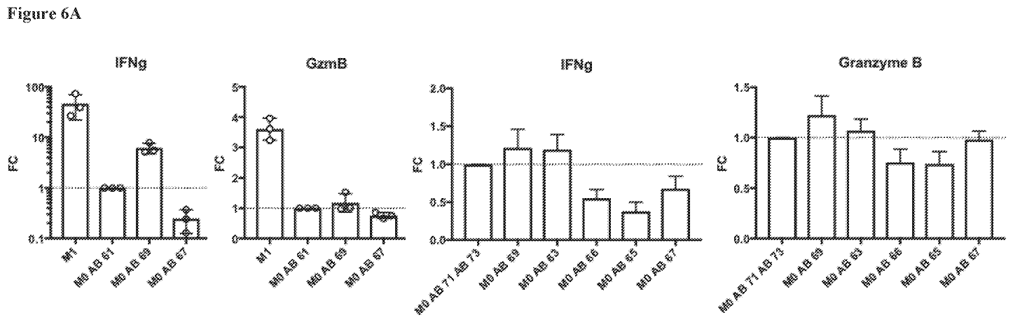

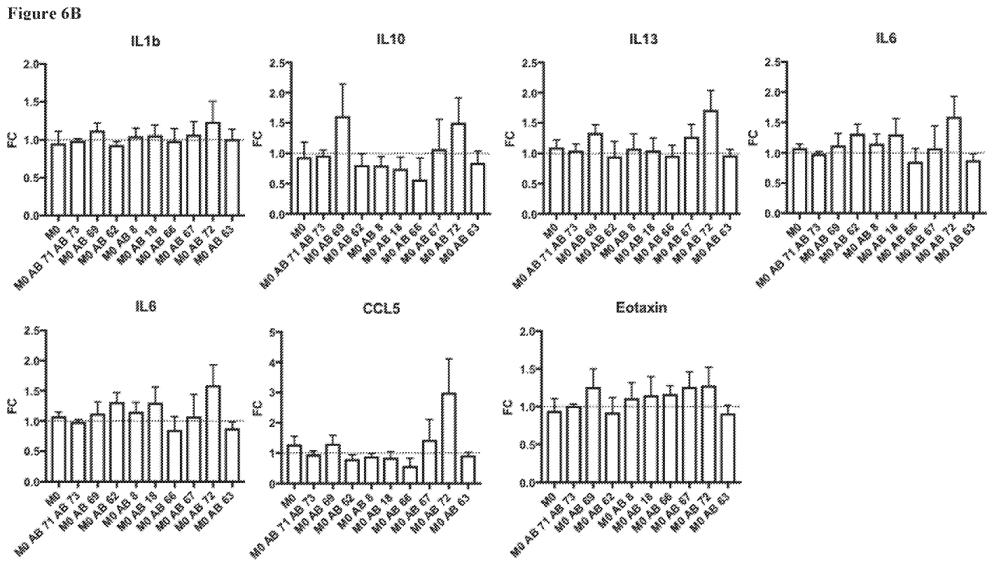

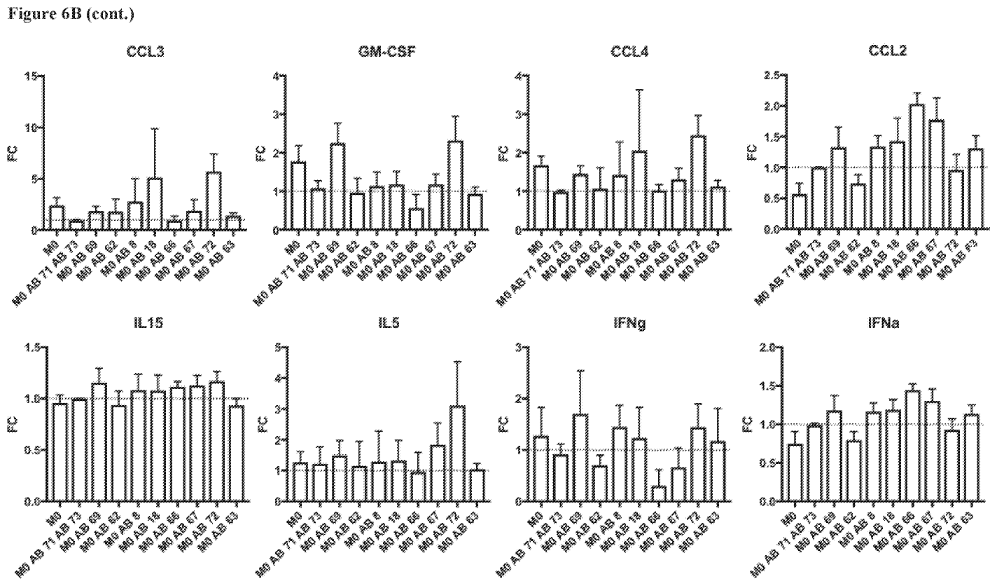

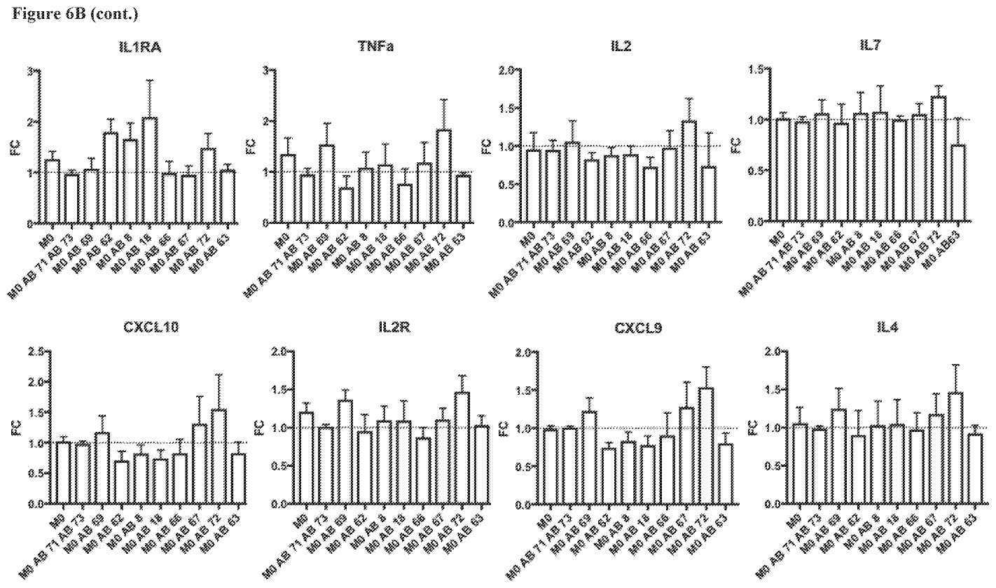

FIG. 6 shows the results from MLR experiments, where two distinct responses are shown. FIG. 6A shows intra-cellular flow staining for IFNγ and Granzyme B from T cells. Intracellular staining was conducted as described above with the inclusion of a fixation and permeabilization step. Staining for intracellular epitopes was performed using the BD Cytofix/Cytoperm™ Fixation/Permeabilization Kit according to the manufacturer's protocol. In this assay, some mAbs were able to effect a decrease in T cell function. Given that the MLR assay mimics an inflammatory GVHD type reaction, the results demonstrate a potential for using the macrophage associated targets in a setting where decreasing inflammation is warranted or desired. FIG. 6B shows the other distinct response with mAbs that have previously demonstrated the ability to drive M2 macrophages to a more M1-like state. An increase in both lymphoid- and myeloid-derived cytokines was determined, which is reminiscent of what was determined in the SEB assay. Both of these assays clearly demonstrate the potential of modulating macrophage associated targets to alter both macrophage function, as well as T cell function, and thereby elicit a concerted immune response.

### Example 5: Macrophage Inflammatory Phenotype Modulation by Modulating Validated Targets as Compared to Immune Checkpoint Inhibitor Treatment

Checkpoint inhibitors, e.g., PD-1 blocking antibodies, are currently the gold standard immuno-oncology therapeutic. Traditionally checkpoint inhibitors have focused on blocking inhibitory receptors expressed directly on T cells and thereby directly increasing T cell activity against tumors. The targets that have been shown to be validated above have been shown to alter first the phenotype and function of macrophages and then lead to a concerted immune response, including T cell activation. Therefore, the ability of these macrophage associated targets to function equal to or better than checkpoint inhibitors or potentially combine with needs to be confirmed.

The ability of validated targets to function equal to or better than checkpoint inhibitors, including in instances where checkpoint inhibitors do not work, is demonstrated in FIG. 5. In this assay, two donors are shown. The assay was performed as described in SEB assay above. For example, PD1 blockade (e.g., using pembrolizumab) in donor 5 resulted in an increase in T cell-specific response as indicated by IFNg production, whereas in donor 2 there was no increase in T cell activity in the presence of PD1 blockade. In both donor 2 and donor 5, specific antibodies were able to induce both T cell- and myeloid cell-specific responses. Furthermore, the combination of PD1 blockade with validated target antibodies led to an additive response, indicating efficacy of anti-target therapeutics use, either alone or in combination with checkpoint inhibitors.

### Example 6: Expression and Function of Macrophage-Associated Targets in the Tumor Microenvironment

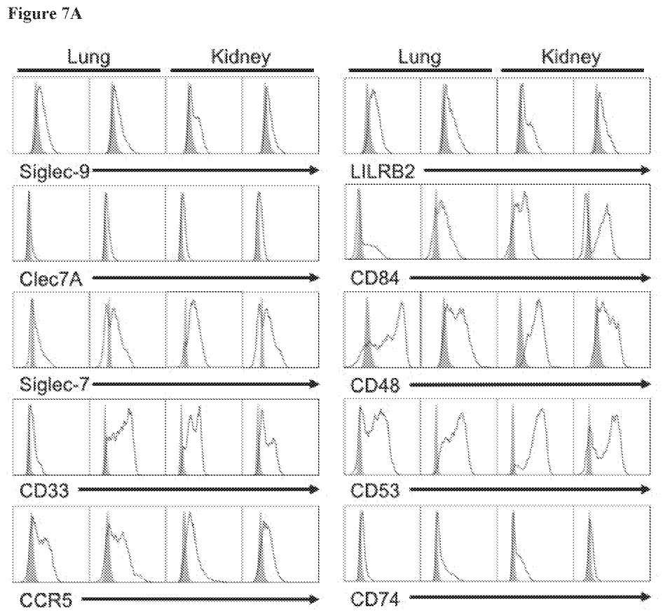

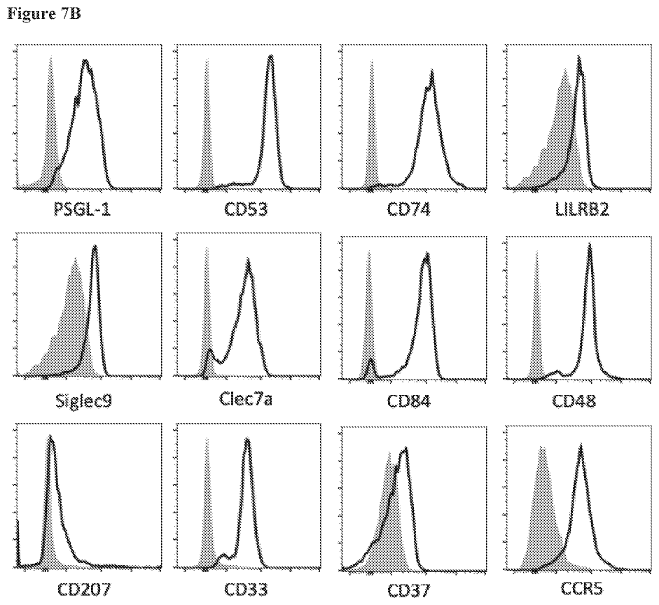

Validated macrophage-associated targets were tested for their expression on tumor associated macrophages (TAMs) (FIG. 7). Flow cytometry was performed as described above. Macrophage-associated targets were demonstrated to be expressed on TAMs, such as those from lung, tumors, kidney tumors and the cellular constituents of ascites fluid from gynecological cancers (FIG. 7B). Consistent robust expression of these validated macrophage-associated targets is seen across different tumor types and systems.

The above in vitro systems clearly show the ability of these validated macrophage-associated targets to alter macrophage function as well as complex multi-cellular assays including T cells. These data were further confirmed using patient tumor material in an ex vivo culture system. This type of system represents a close and generally accepted surrogate to a human in vivo study, therefore providing potent evidence of therapeutic benefit. The macrophage-associated targets were further tested for their regulation of the biology of macrophages in a tissue environment using several independent systems.

One system used for this purpose was a dissociated tumor assay. Dissociated tumors contain all the various cell populations present in the tumor microenvironment, including, for example, tumor cells, immune cells, and support cells. These viable single cell suspensions are useful for many applications and allow for the normalization of cell number and composition within each replicate of an experiment. For performing dissociated tumor experiments upon acquisition of fresh tumor tissue (less than 24 hrs), surrounding fat, fibrous areas, and necrotic areas were removed from a tumor sample using scissors and scalpels. The tumor was cut into small pieces of 2-4 mm³. The Tumor Dissociation Kit enzyme mix (MACS Miltenyi Biotec) was prepared according to the manufacturer's protocol. Tumor pieces and dissociation enzymes were transferred into 5 ml Snaplock Microcentrifuge tubes and the tissue was minced using a pair of straight scissors. Tubes were placed in a 37° C. shaker at 200-250 rpm for 45 minutes to 1 hour. At the end of the incubation time, the digested tumor was filtered through 40 uM cell strainers into 50 mL Falcon™ conical centrifuge tubes. The tube was filled with cold 2%-5% FBS/PBS mix to stop the digestion. All of the remaining steps were performed on ice. In particular, the tube was centrifuged for 5 minutes at 300 g, the supernatant was discarded, and the cells were washed twice with cold 2% to 5% FBS/PBS mix. Following the last wash, the cells were resuspended in 1 to 5 ml of cold 2% to 5% FBS/PBS mix and a cell count was performed. Approximately 300 k to 400 k cells were plated in each well of 6-well culture plates containing 1 ml of media (DMEM with L-glutamine, 4.5 g/L glucose and sodium pyruvate (Fisher Scientific), Gibco™ GutaMAX® supplement (Fisher Scientific), Gibco™ MEM Non-Essential Amino Acids Solution (Fisher Scientific), 2-mercaptoethanol (55 mM) (Fisher Scientific), heat inactivated from human male AB plasma (Sigma-Aldrich, Inc), bovine calf serum heat inactivated (BioFluid Technologies), 100× pen/strep, and human M-CSF (BioLegend)) and indicated antibodies. All antibodies were added at a concentration of 10 ug/mL. Plates were incubated in a cell culture incubator at 37° C. with 5% CO₂ for 24 or 48 hrs. At the end of the study, cytokines/chemokines in the culture supernatant were measured using the Invitrogen™ Luminex™ Cytokine Human Magnetic 25-Plex Panel according to the manufacturer's instructions.

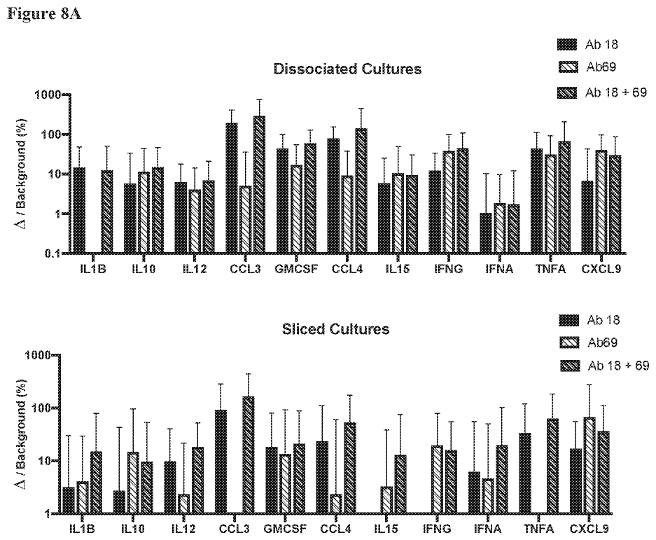

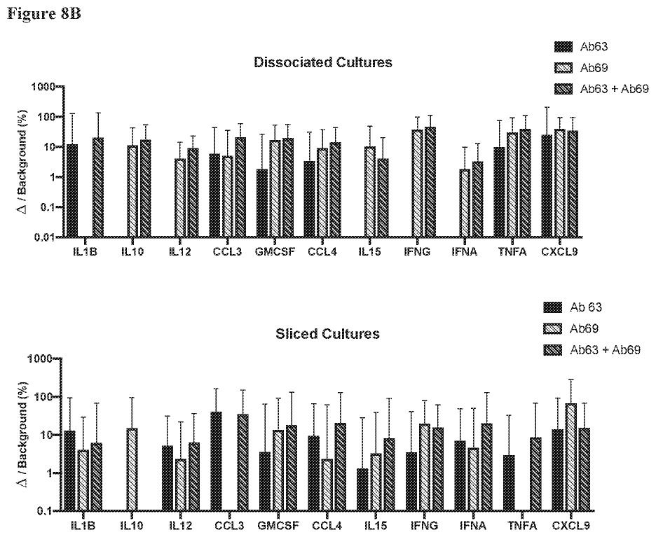

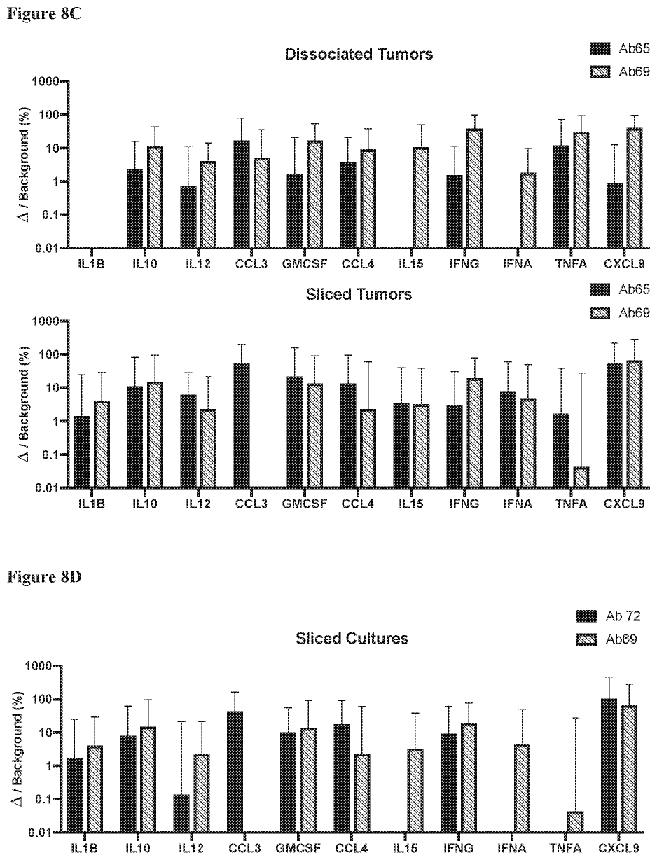

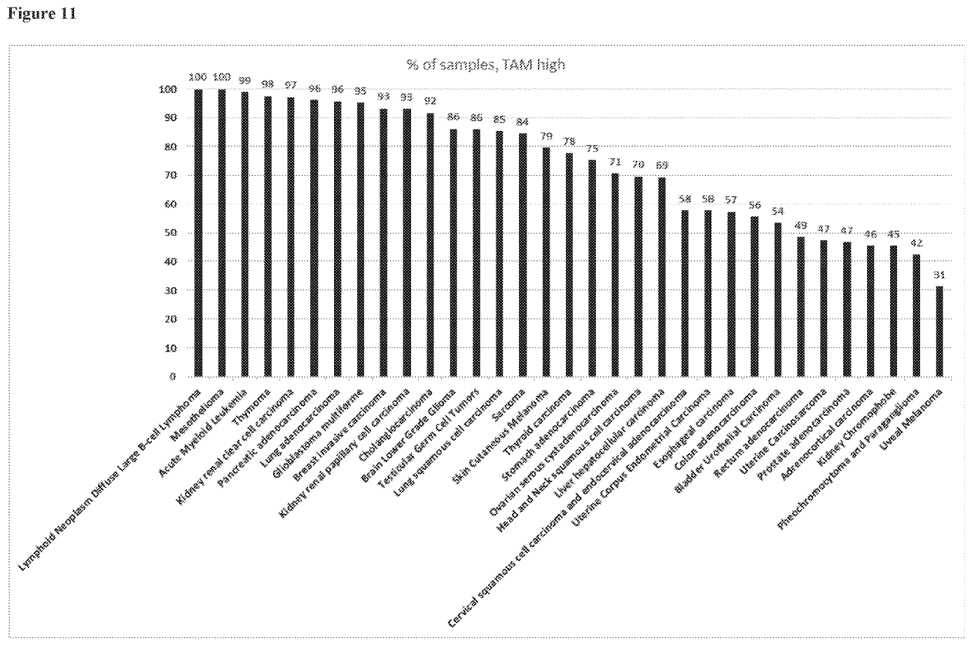

Tumor samples treated with specific antibodies, as well as pembrolizumab (KEYTRUDA®) are shown in FIG. 8. The data described herein presents 34 (FIGS. 8A-8C) or 11 (FIG. 8D) individual tumors comprising 6 tumor types. The data demonstrate the ability of antibodies against validated macrophage-associated targets to induce M1-like pro-inflammatory function within TAMs, as shown by the production of TNFα, GM-CSF, and IL-12. This assay also demonstrates the potential to elicit a concerted immune response within a tumor beyond macrophage function as shown by the production of cytokines such as IFNg. This is a significant finding as it demonstrates that administering a single agent directed at a surface receptor, in an immunosuppressive environment, modulating myeloid cells and, in turn, effecting a T cell response, thereby demonstrating the potential to drive an M2- to M1-like differentiation in a tumor and induce an anti-tumor response. This potent concerted immune response is directly compared to pembrolizumab (KEYTRUDA®) response and the results demonstrate that modulating the macrophage-associated targets can have a dramatically more potent response demonstrating efficacious use of a single agent therapy directed at validated targets. Additionally, the effect of macrophage-associated targets is seen in cases where pembrolizumab (KEYTRUDA®) is not obvious, paving the way to expanding the responder population. Selected antibodies have also been combined with pembrolizumab (KEYTRUDA®) and show an additive effect demonstrating combination therapies to increase efficacy and/or overcome resistance to checkpoint therapies. It should also be noted that pembrolizumab (KEYTRUDA®) induces immunosuppressive IL-10 in addition to stimulating IFNg production, which can limit its activity, while our target engagement has not stimulated immunosuppressive IL-10. There are also clear instances of significant upregulation of chemokines, which is believed to recruit fresh immune cells to further perpetuate an anti-tumor immune response.

A second system was used to further confirm the results of the dissociated tumor assays. Briefly, dissociated tumor assays have the advantage of being able to normalize the number of cells in every condition performed. They also have the potential downside of losing the tumor structure and non-cellular stromal components. In order to address this question and demonstrate the ability of validated targets to induce a concerted immune response in an intact tumor, tissue slice cultures were performed. Upon acquisition of fresh tumor tissue sample (less than 24 hrs), surrounding fat, fibrous, and necrotic areas were removed from the tumor sample as much as possible using scissors and scalpels. The tissue was embedded in the center of a tissue mold using 4% agarose. After solidifying, the agarose block was dislodged, and the mold was glued to a Leica VT1000 S microtome tissue holder and sectioned into 300 to 400 micron sections using the Leica VT1000 S microtome. Tissue slices were transferred to a cell culture insert and then into a well of a 6-well cell culture plate containing media (DMEM with L-Glutamine, 4.5 g/L glucose and sodium pyruvate (Fisher Scientific), Gibco™ GlutaMAX™ Supplement (Fisher Scientific), Gibcom MEM Non-Essential Amino Acids Solution (Fisher Scientific), 2-mercaptoethanol (55 mM) (Fisher Scientific), heat inactivated from human male AB plasma (Sigma-Aldrich, Inc), bovine calf serum heat inactivated (BioFluid Technologies), 100× pen/strep, and human M-CSF (BioLegend)) and indicated antibodies. All antibodies were added at a concentration of 10 ug/mL. The slices were then incubated at 37° C. with 5% CO₂ for 1-5 days. At the end of the study, efficacy was evaluated by analyzing cytokine/chemokine amounts secreted into the culture supernatant measured using the Invitrogen™ Luminex™ Cytokine Human Magnetic 25-Plex Panel according to the manufacturer's instructions. If a tumor tissue sample was not appropriate to be sliced using a Leica VT1000 S microtome, it was cut using scalpels as thinly as possible and the slices were subsequently treated as described above. For all samples, a slice was taken prior to treatment and utilized for immunophenotyping. This slice was dissociated as described above and stained for flow cytometry analyses as described above.

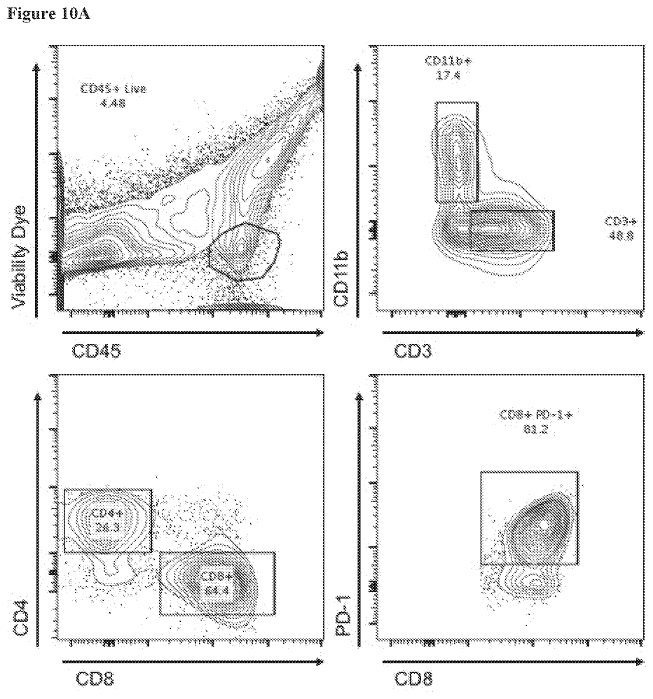

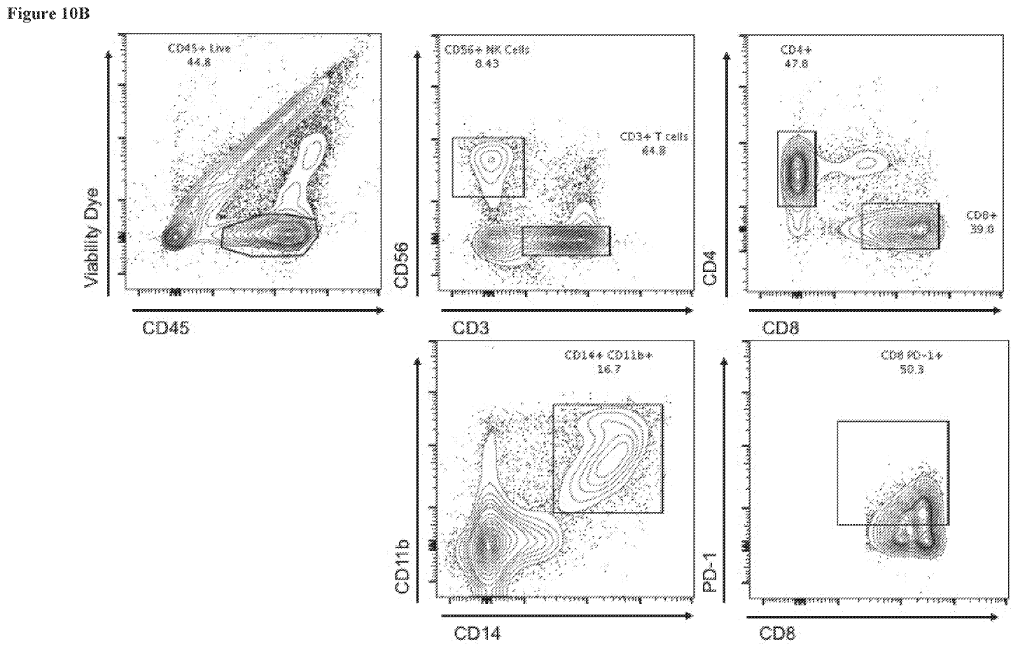

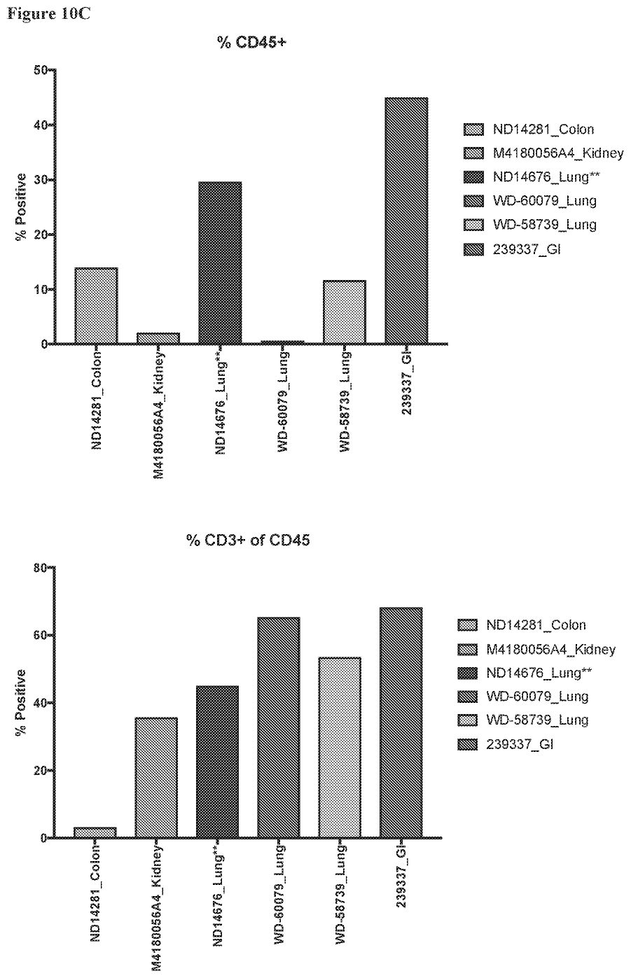

FIG. 8 demonstrates the function of selected antibodies across multiple tumor types and donors. In this figure, results from 6 different tumors, including kidney, lung, and GI tumors, were combined for each treatment. The induction of a pro-inflammatory response was consistent and significant, thereby demonstrating broad function and applicability. These various tumors are composed of both highly infiltrated examples and minimally infiltrated tumors, as demonstrated in FIG. 10A-C. These results further demonstrate that antibodies against validated targets can induce a potent pro-inflammatory, and likely anti-tumor, immune response within the tumor microenvironment. This effect can be equal to or greater than that of pembrolizumab (KEYTRUDA®), including in tumors with very low or absent T cell infiltrate.

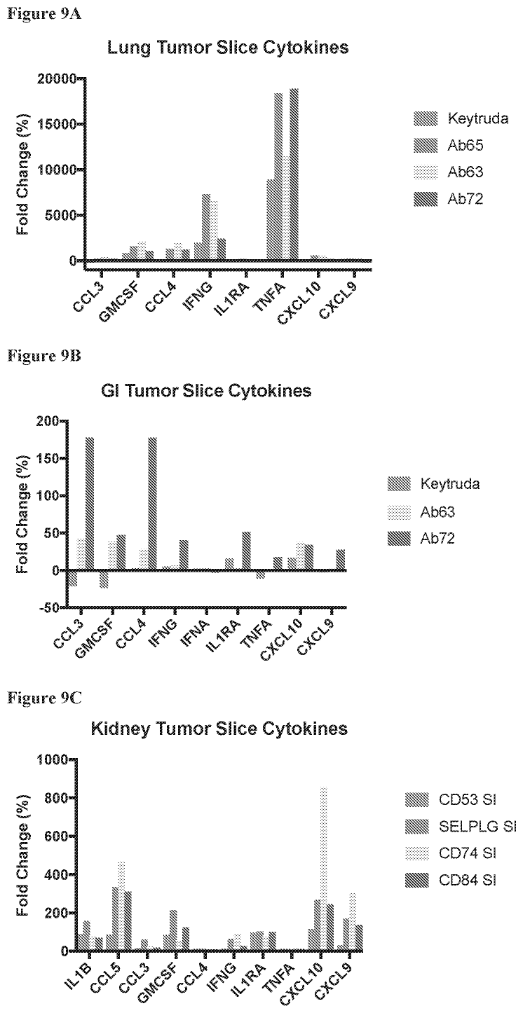

FIGS. 9A-9C show the cytokine production resulting from 3 separate tumor types treated with antibodies, as well as with validated siRNAs, described above. In all 3 independent samples, the modulation of the validated macrophage-associated targets with either antibody or siRNA induced a pro-inflammatory immune response, including both myeloid and lymphoid specific responses.

FIGS. 9A and 9B also demonstrate two different responses with regard to PD1 blockade. The lung tumor assay (FIG. 9A) showed a response to pembrolizumab (KEYTRUDA®) with anticipated cytokines, such as IFNg and TNFα, being produced. Selected antibodies to targets in this tumor elicited an equal or greater response than that of pembrolizumab (KEYTRUDA®) treatment. The GI tumor results shown in FIG. 9B do not provide a response elicited by pembrolizumab (KEYTRUDA®). Importantly, immunophenotyping of this tumor showed a significant population of PD1+CD8 T cells (FIG. 10A)—a necessary, but not sufficient, feature for the PD-1 response. In this tumor, the antibodies shown were still able to elicit a response from both myeloid cells, as demonstrated by the production of CCL3, CCL4, and GM-CSF, and T cells, as demonstrated by the production of IFNg.

Thus, the representative examples of at least the following are provided: a) in T cell infiltrated tumors, validated macrophage-associated target (VTx) monotherapy or combination therapy leads to a stronger activation of T cells and macrophages than pembrolizumab (KEYTRUDA®); b) in T cell infiltrated tumors, VTx monotherapy or combination can lead to activation of both T cells and macrophages when pembrolizumab (KEYTRUDA®) does not; c) in tumors with none/very poor T cell infiltration, VTx monotherapy or combination can lead to broad inflammatory changes in the tumor microenvironment; and clear achievement of additivity and potential synergy to immune checkpoint inhibition.

Without being bound by theory, it is believed that macrophages as influenced by their inflammatory state modulate immune responses due to their presence not limited to antigen-specific interactions like other immune cells. Accordingly, the ability to elicit an immune response across all of these levels of immune infiltration demonstrates a wide applicability across tumor types and immune status. About a quarter of all human cancers are considered to be an immune desert, whereas the remainder are infiltrated by immune cells. While only a minority of those tumors have a full immune surveillance by T cells, all of them have a significant infiltration by both surveilling (pro-inflammatory) and tumor supporting (pro-tumorigenic) macrophages. FIG. 11 shows a distribution of macrophage-infiltrating tumors across cancer types of the large public dataset of human cancers (TCGA, The Cancer Genome Atlas, 2017 version, processed and distributed by OmicSoft/Qiagen). Tumor infiltration is measured by the presence of a canonical myeloid marker CD11b above the cutoff. The cutoff is defined as a first quartile of the CD11b mRNA expression distribution across all primary tumors in the dataset. These macrophage-infiltrating tumors are believed to be particularly useful for modulation according to the compositions and methods described herein.

### Biological Deposits

Representative materials of the present invention were deposited in the American Type Culture Collection (ATCC) on Jun. 4, 2019 and Jun. 20, 2019 by Verseau Therapeutics, Inc. In particular, monoclonal antibodies deposited as individual deposits having the following names: “13H10” (PTA-125944), “13J19” (PTA-125945), “18F02” (PTA-125946), and “19I01” (PTA-125943), and having identifying characteristics shown in Table 9 and the Examples, were deposited in the ATCC on Jun. 4, 2019 by Verseau Therapeutics, Inc. under the provisions of the Budapest Treaty on the International Recognition of the Deposit of Microorganisms for the Purpose of patent Procedure and Regulations thereunder (Budapest Treaty). Similarly, monoclonal antibodies deposited as individual deposits having the following names: “1F01” (PTA-126025), “3C01” (PTA-126026), “10M15” (PTA-126027), “4H23” (PTA-126028), “7A12” (PTA-126029), and “7F19” (PTA-126030), and having identifying characteristics shown in Table 9 and the Examples, were deposited in the ATCC on Jun. 20, 2019 by Verseau Therapeutics, Inc. under the provisions of the Budapest Treaty on the International Recognition of the Deposit of Microorganisms for the Purpose of patent Procedure and Regulations thereunder (Budapest Treaty). This assures maintenance of a viable deposit for 30 years from the date of deposit. The deposit will be made available by ATCC under the terms of the Budapest Treaty, and subject to an agreement between Verseau Therapeutics, Inc. and ATCC, which assures permanent and unrestricted availability of the deposit to the public upon issuance of the pertinent U.S. patent or upon laying open to the public of any U.S. or foreign patent application, whichever comes first, and assures availability of the deposit to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a deposit should be lost or destroyed, the materials will be promptly replaced on notification with another of the same. Availability of the deposited material is not to be construed as a license to practice the invention in contravention of the rights granted under the authority of any government in accordance with its patent laws.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### EQUIVALENTS AND SCOPE

The details of one or more embodiments encompassed by the present invention are set forth in the description above. Although the preferred materials and methods have been described above, any materials and methods similar or equivalent to those described herein can be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which this invention belongs. In the case of conflict, the present description provided above will control.

Those skilled in the art will recognize or be able to ascertain using no more than routine experimentation, many equivalents to the specific embodiments encompassed by the present invention described herein. The scope of the present invention is not intended to be limited to the description provided herein and such equivalents are intended to be encompassed by the appended claims.

The articles “a” and “an” are used herein to refer to one or to more than one (i.e. to at least one) of the grammatical object of the article unless indicated to the contrary or otherwise evident from the context. By way of example, “an element” means one element or more than one element. Claims or descriptions that include “or” between one or more members of a group are considered satisfied if one, more than one, or all of the group members are present in, employed in, or otherwise relevant to a given product or process unless indicated to the contrary or otherwise evident from the context. The present invention includes embodiments in which exactly one member of the group is present in, employed in, or otherwise relevant to a given product or process. The present invention also includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments encompassed by the present invention, to the tenth of the unit of the lower limit of the range, unless the context clearly dictates otherwise.

In addition, it is to be understood that any particular embodiment of the present invention that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions encompassed by the present invention (e.g., any antibiotic, therapeutic or active ingredient, any method of production; any method of use; etc.) can be excluded from any one or more claims, for any reason, whether or not related to the existence of prior art.

It is to be understood that the words which have been used are words of description rather than limitation, and that changes can be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present invention.

What is claimed is:

1. A method of generating monocytes and/or macrophages having an increased inflammatory phenotype after contact with at least one agent comprising contacting monocytes and/or macrophages with an effective amount of the at least one agent, wherein the at least one agent is a) an agent that downregulates the copy number, amount, and/or activity of at least one target listed in Table 1 and/or b) an agent that upregulates the copy number, amount, and/or activity of at least one target listed in Table 2.

2. The method of claim 1, wherein the monocytes and/or macrophages having an increased inflammatory phenotype exhibit one or more of the following after contact with the agent or agents:
a) increased expression and/or secretion of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α);
b) decreased expression and/or secretion of CD206, CD163, CD16, CD53, VSIG4, PSGL-1, TGFb and/or IL-10;
c) increased secretion of at least one cytokine or chemokine selected from the group consisting of IL-1β, TNF-α, IL-12, IL-18, GM-CSF, CCL3, CCL4, and IL-23;
d) increased ratio of expression of IL-1$, IL-6, and/or TNF-α to expression of IL-10;
e) increased CD8+ cytotoxic T cell activation;
f) increased recruitment of CD8+ cytotoxic T cell activation;
g) increased CD4+ helper T cell activity;
h) increased recruitment of CD4+ helper T cell activity;
i) increased NK cell activity;
j) increased recruitment of NK cell;
k) increased neutrophil activity;
l) increased macrophage activity; and/or
m) increased spindle-shaped morphology, flatness of appearance, and/or number of dendrites, as assessed by microscopy.

3. The method of claim 1 or 2, wherein the monocytes and/or macrophages contacted with the agent or agents are comprised within a population of cells and the agent increase the number of Type 1 and/or M1 macrophages, and/or decrease the number of Type 2 and/or M2 macrophages, in the population of cells.

4. The method of any one of claims 1-3, wherein the monocytes and/or macrophages contacted with the agent or agents are comprised within a population of cells and the agent or agents increases the ratio of i) to ii), wherein i) is Type 1 and/or M1 macrophages and ii) is Type 2 and/or M2 macrophages in the population of cells.

5. A method of generating monocytes and/or macrophages having a decreased inflammatory phenotype after contact with at least one agent comprising contacting monocytes and/or macrophages with an effective amount of the at least one agent, wherein the agent is a) an agent that upregulates the copy number, amount, and/or activity of at least one target listed in Table 1 and/or b) an agent that downregulates the copy number, amount, and/or activity of at least one target listed in Table 2.

6. The method of claim 5, wherein the monocytes and/or macrophages having the decreased inflammatory phenotype exhibit one or more of the following after contact with the agent or agents:
a) decreased expression and/or secretion of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α);
b) increased expression and/or secretion of CD206, CD163, CD16, CD53, VSIG4, PSGL-1 and/or IL-10;
c) decreased secretion of at least one cytokine selected from the group consisting of IL-1β, TNF-α, IL-12, IL-18, and IL-23;
d) decreased ratio of expression of IL-1β, IL-6, and/or TNF-α to expression of IL-10;
e) decreased CD8+ cytotoxic T cell activation;
f) decreased CD4+ helper T cell activity;
g) decreased NK cell activity;
h) decreased pro-inflammatory neutrophil activity,
i) decreased macrophage activity; and/or
j) decreased spindle-shaped morphology, flatness of appearance, and/or number of dendrites, as assessed by microscopy.

7. The method of claim 5 or 6, wherein the monocytes and/or macrophages contacted with the agent or agents are comprised within a population of cells and the agent decrease the number of Type 1 and/or M1 macrophages, and/or increase the number of Type 2 and/or M2 macrophages, in the population of cells.

8. The method of any one of claims 5-7, wherein the monocytes and/or macrophages contacted with the agent or agents are comprised within a population of cells and the agent or agents decrease the ratio of i) to ii), wherein i) is Type 1 and/or M1 macrophages and ii) is Type 2 and/or M2 macrophages in the population of cells.

9. The method of any one of claims 1-8, wherein the agent or agents that downregulate the copy number, amount, and/or activity of at least one target listed in Table 1 and/or Table 2 is a small molecule inhibitor, CRISPR guide RNA (gRNA), RNA interfering agent, antisense oligonucleotide, single-stranded nucleic acid, double-stranded nucleic acid, aptamer, ribozyme, DNAzyme, peptide, peptidomimetic, antibody, intrabody, or cells.

10. The method of claim 9, wherein the RNA interfering agent is a small interfering RNA (siRNA), a small hairpin RNA (shRNA), microRNA (miRNA), or a piwi-interacting RNA (piRNA).

11. The method of any one of claims 1-8, wherein the agent or agents that downregulate the copy number, amount, and/or activity of at least one target listed in Table 1 and/or Table 2 comprises an antibody and/or intrabody, or an antigen binding fragment thereof, which specifically binds to the at least one target listed in Table 1 and/or Table 2.

12. The method of claim 11, wherein the antibody and/or intrabody, or antigen binding fragment thereof, is camelid, murine, chimeric, humanized, human, detectably labeled, comprises an effector domain, comprises an Fc domain, and/or is selected from the group consisting of Fv, Fav, F(ab′)2, Fab′, dsFv, scFv, sc(Fv)2, and diabodies fragments.

13. The method of claim 11 or 12, wherein the antibody and/or intrabody, or antigen binding fragment thereof, is conjugated to a cytotoxic agent.

14. The method of claim 13, wherein the cytotoxic agent is selected from the group consisting of a chemotherapeutic agent, a biologic agent, a toxin, and a radioactive isotope.

15. The method of any one of claims 1-8, wherein the agent or agents that upregulate the copy number, amount, and/or activity of at least one target listed in Table 1 and/or Table 2 is a nucleic acid molecule encoding the one or more targets listed in Table 1 and/or Table 2 or fragment thereof, a polypeptide of the one or more targets listed in Table 1 and/or Table 2 or fragment(s) thereof, an activating antibody and/or intrabody that binds to the one or more targets listed in Table 1 and/or Table 2, or a small molecule that binds to the one or more targets listed in Table 1 and/or Table 2.

16. The method of any one of claims 1-15, wherein the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target.

17. The method of any one of claims 1-16, wherein the monocytes and/or macrophages are contacted in vitro or ex vivo.

18. The method of claim 17, wherein the monocytes and/or macrophages are primary monocytes and/or primary macrophages.

19. The method of claim 17 or 18, wherein the monocytes and/or macrophages are purified and/or cultured prior to contact with the agent or agents.

20. The method of any one of claims 1-16, wherein the monocytes and/or macrophages are contacted in vivo.

21. The method of claim 20, wherein the monocytes and/or macrophages are contacted in vivo by systemic, peritumoral, or intratumoral administration of the agent.

22. The method of claim 20 or 21, wherein the monocytes and/or macrophages are contacted in a tissue microenvironment.

23. The method of any one of claims 1-22, further comprising contacting the monocytes and/or macrophages with at least one immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus.

24. A composition comprising i) a monocyte and/or macrophage generated according to a method of any one of claims 1-23 and/or ii) an siRNA for downregulating the amount and/or activity of at least one target listed in Table 1 and/or Table 2.

25. A method of increasing an inflammatory phenotype of monocytes and/or macrophages in a subject after contact with at least one agent comprising administering to the subject an effective amount of the at least one agent, wherein the at least one agent is a) an agent that downregulates the copy number, amount, and/or activity of at least one target listed in Table 1 in or on the monocytes and/or macrophages, and/or b) an agent that upregulates the copy number, amount, and/or activity of at least one target listed in Table 2 in or on the monocytes and/or macrophages.

26. The method of claim 25, wherein the monocytes and/or macrophages having the increased inflammatory phenotype exhibit one or more of the following after contact with the agent or agents:
a) increased expression and/or secretion of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α);
b) decreased expression and/or secretion of CD206, CD163, CD16, CD53, VSIG4, PSGL-1 and/or IL-10;
c) increased secretion of at least one cytokine selected from the group consisting of IL-1β, TNF-α, IL-12, IL-18, and IL-23;
d) increased ratio of expression of IL-1β, IL-6, and/or TNF-α to expression of IL-10;
e) increased CD8+ cytotoxic T cell activation;
f) increased CD4+ helper T cell activity;
g) increased NK cell activity;
h) increased neutrophil activity;
i) increased macrophage activity; and/or
j) increased spindle-shaped morphology, flatness of appearance, and/or number of dendrites, as assessed by microscopy.

27. The method of claim 25 or 26, wherein the agent or agents increase the number of Type 1 and/or M1 macrophages, decrease the number of Type 2 and/or M2 macrophages, and/or increase the ratio of i) to ii), wherein i) is Type 1 and/or M1 macrophages and ii) is Type 2 and/or M2 macrophages, in the subject.

28. The method of any one of claims 25-27, wherein the number and/or activity of cytotoxic CD8+ T cells in the subject is increased after administration of the agent or agents.

29. A method of decreasing an inflammatory phenotype of monocytes and/or macrophages in a subject after contact with at least one agent comprising administering to the subject an effective amount of the at least one agent, wherein the at least one agent is a) an agent that upregulates the copy number, amount, and/or activity of at least one target listed in Table 1 in or on the monocytes and/or macrophages, and/or b) an agent that downregulates the copy number, amount, and/or activity of at least one target listed in Table 2 in or on the monocytes and/or macrophages.

30. The method of claim 29, wherein the monocytes and/or macrophages having the decreased inflammatory phenotype exhibit one or more of the following after contact with the agent or agents:
a) decreased expression and/or secretion of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α);
b) increased expression and/or secretion of CD206, CD163, CD16, CD53, VSIG4, PSGL-1 and/or IL-10;
c) decreased secretion of at least one cytokine selected from the group consisting of IL-1s, TNF-α, IL-12, IL-18, and IL-23;
d) decreased ratio of expression of IL-1β, IL-6, and/or TNF-α to expression of IL-10;
e) decreased CD8+ cytotoxic T cell activation;
f) decreased CD4+ helper T cell activity;
g) decreased NK cell activity;
h) decreased neutrophil activity;
i) decreased macrophage activity; and/or
j) decreased spindle-shaped morphology, flatness of appearance, and/or number of dendrites, as assessed by microscopy.

31. The method of claim 29 or 30, wherein the agent or agents decrease the number of Type 1 and/or M1 macrophages, increase the number of Type 2 and/or M2 macrophages, and/or decrease the ratio of i) to ii), wherein i) is Type 1 and/or M1 macrophages and ii) is Type 2 and/or M2 macrophages, in the subject.

32. The method of any one of claims 29-31, wherein the number and/or activity of cytotoxic CD8+ T cells in the subject is decreased after administration of the agent.

33. The method of any one of claims 25-32, wherein the agent that downregulates the copy number, amount, and/or activity of at least one target listed in Table 1 and/or Table 2 is a small molecule inhibitor, CRISPR guide RNA (gRNA), RNA interfering agent, antisense oligonucleotide, peptide or peptidomimetic inhibitor, aptamer, antibody, intrabody, or cells.

34. The method of claim 33, wherein the RNA interfering agent is a small interfering RNA (siRNA), a small hairpin RNA (shRNA), microRNA (miRNA), or a piwi-interacting RNA (piRNA).

35. The method of any one of claims 25-32, wherein the agent that downregulates the copy number, amount, and/or activity of at least one target listed in Table 1 and/or Table 2 comprises an antibody and/or intrabody, or an antigen binding fragment thereof, which specifically binds to the at least one target listed in Table 1 and/or Table 2.

36. The method of claim 35, wherein the antibody and/or intrabody, or antigen binding fragment thereof, is camelid, murine, chimeric, humanized, human, detectably labeled, comprises an effector domain, comprises an Fc domain, and/or is selected from the group consisting of Fv, Fav, F(ab′)2, Fab′, dsFv, scFv, sc(Fv)2, and diabodies fragments.

37. The method of claim 35 or 36, wherein the antibody and/or intrabody, or antigen binding fragment thereof, is conjugated to a cytotoxic agent.

38. The method of claim 37, wherein the cytotoxic agent is selected from the group consisting of a chemotherapeutic agent, a biologic agent, a toxin, and a radioactive isotope.

39. The method of any one of claims 25-32, wherein the agent that upregulates the copy number, amount, and/or activity of at least one target listed in Table 1 and/or Table 2 is a nucleic acid molecule encoding the one or more targets listed in Table 1 and/or Table 2 or fragment thereof, a polypeptide of the one or more targets listed in Table 1 and/or Table 2 or fragment(s) thereof, an activating antibody and/or intrabody that binds to the one or more targets listed in Table 1 and/or Table 2, or a small molecule that binds to the one or more targets listed in Table 1 and/or Table 2.

40. The method of any one of claims 25-39, wherein the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target.

41. The method of claim 40, wherein the agent or agents are administered in vivo by systemic, peritumoral, or intratumoral administration of the agent.

42. The method of claim 40 or 41, wherein the agent or agents contact the monocytes and/or macrophages in a tissue microenvironment.

43. The method of any one of claims 25-42, further comprising contacting the monocytes and/or macrophages with at least one immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus.

44. A method of increasing inflammation in a subject comprising administering to the subject an effective amount of a) monocytes and/or macrophages contacted with at least one agent to downregulate the copy number, amount, and/or activity of at least one target listed in Table 1 and/or b) monocytes and/or macrophages contacted with at least one agent to upregulate the copy number, amount, and/or activity of at least one target listed in Table 2.

45. The method of claim 44, wherein the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target.

46. The method of claim 44 or 45 wherein the monocytes and/or macrophages are genetically engineered, autologous, syngeneic, or allogeneic relative to the subject's monocytes and/or macrophages.

47. The method of any one of claims 44-46, wherein the monocytes and/or macrophages contacted with the at least one agent of a) are different from the monocytes and/or macrophages contacted with the at least one agent of b).

48. The method of any one of claims 44-46, wherein the monocytes and/or macrophages contacted with the at least one agent of a) are the same as the monocytes and/or macrophages contacted with the at least one agent of b).

49. The method of any one of claims 44-48, wherein the agent or agents are administered systemically, peritumorally, or intratumorally.

50. A method of decreasing inflammation in a subject comprising administering to the subject an effective amount of a) monocytes and/or macrophages contacted with at least one agent to upregulate the copy number, amount, and/or activity of at least one target listed in Table 1 and/or b) monocytes and/or macrophages contacted with at least one agent to downregulate the copy number, amount, and/or activity of at least one target listed in Table 2.

51. The method of claim 50, wherein the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target.

52. The method of claim 50 or 51, wherein the monocytes and/or macrophages are genetically engineered, autologous, syngeneic, or allogeneic relative to the subject's monocytes and/or macrophages.

53. The method of any one of claims 50-52, wherein the monocytes and/or macrophages contacted with the at least one agent of a) are different from the monocytes and/or macrophages contacted with the at least one agent of b).

54. The method of any one of claims 50-52, wherein the monocytes and/or macrophages contacted with the at least one agent of a) are the same as the monocytes and/or macrophages contacted with the at least one agent of b).

55. The method of any one of claims 50-54, wherein the agent or agents are administered systemically, peritumorally, or intratumorally.

56. A method of sensitizing cancer cells in a subject to cytotoxic CD8+ T cell-mediated killing and/or immune checkpoint therapy comprising administering to the subject a therapeutically effective amount of a) at least one agent that downregulates the copy number, amount, and/or activity of at least one target listed in Table 1 in or on monocytes and/or macrophages and/or b) at least one agent that upregulates the copy number, amount, and/or activity of at least one target listed in Table 2 in or on monocytes and/or macrophages.

57. The method of claim 56, comprising administering at least one agent that downregulates the copy number, amount, and/or activity of at least one target listed in Table 1.

58. The method of claim 57, wherein the agent is a small molecule inhibitor, CRISPR guide RNA (gRNA), RNA interfering agent, antisense oligonucleotide, peptide or peptidomimetic inhibitor, aptamer, antibody, intrabody, or cells.

59. The method of claim 58, wherein the RNA interfering agent is a small interfering RNA (siRNA), a small hairpin RNA (shRNA), microRNA (miRNA), or a piwi-interacting RNA (piRNA).

60. The method of claim 58, wherein the agent comprises an antibody and/or intrabody, or an antigen binding fragment thereof, which specifically binds to the at least one target listed in Table 1.

61. The method of claim 60, wherein the antibody and/or intrabody, or antigen binding fragment thereof, is camelid, murine, chimeric, humanized, human, detectably labeled, comprises an effector domain, comprises an Fc domain, and/or is selected from the group consisting of Fv, Fav, F(ab′)2, Fab′, dsFv, scFv, sc(Fv)2, and diabodies fragments.

62. The method of claim 60 or 61, wherein the antibody and/or intrabody, or antigen binding fragment thereof, is conjugated to a cytotoxic agent.

63. The method of claim 62, wherein the cytotoxic agent is selected from the group consisting of a chemotherapeutic agent, a biologic agent, a toxin, and a radioactive isotope.

64. The method of claim 56, comprising administering at least one agent that upregulates the copy number, amount, and/or activity of at least one target listed in Table 2.

65. The method of claim 64, wherein the agent is a nucleic acid molecule encoding the one or more targets listed in Table 2 or fragment thereof, a polypeptide of the one or more targets listed in Table 2 or fragment(s) thereof, an activating antibody and/or intrabody that binds to the one or more targets listed in Table 2, or a small molecule that binds to the one or more targets listed in Table 2.

66. A method of sensitizing cancer cells in a subject afflicted with a cancer to cytotoxic CD8+ T cell-mediated killing and/or immune checkpoint therapy comprising administering to the subject a therapeutically effective amount of a) monocyte cells and/or macrophage cells contacted with at least one agent to downregulate the copy number, amount, and/or activity of at least one target listed in Table 1 and/or b) monocyte cells and/or macrophage cells contacted with at least one agent to upregulate the copy number, amount, and/or activity of at least one target listed in Table 2.

67. The method of claim 66, wherein the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express the target.

68. The method of claim 66 or 67 wherein the monocytes and/or macrophages are genetically engineered, autologous, syngeneic, or allogeneic relative to the subject's monocytes and/or macrophages.

69. The method of any one of claims 66-68, wherein the monocytes and/or macrophages contacted with the at least one agent of a) are different from the monocytes and/or macrophages contacted with the at least one agent of b).

70. The method of any one of claims 66-68, wherein the monocytes and/or macrophages contacted with the at least one agent of a) are the same as the monocytes and/or macrophages contacted with the at least one agent of b).

71. The method of any one of claims 56-70, wherein the agent or agents are administered systemically, peritumorally, or intratumorally.

72. The method of any one of claims 56-71, further comprising treating the cancer in the subject by administering to the subject at least one immunotherapy, optionally wherein the immunotherapy comprises an immune checkpoint inhibitor, immune-stimulatory agonist, inflammatory agent, cells, a cancer vaccine, and/or a virus.

73. The method of claim 72, wherein the immune checkpoint is selected from the group consisting of PD-1, PD-L1, PD-L2, and CTLA-4.

74. The method of claim 73, wherein the immune checkpoint is PD-1.

75. The method of any one of claims 56-74, wherein the agent or agents reduce the number of proliferating cells in the cancer and/or reduce the volume or size of a tumor comprising the cancer cells.

76. The method of any one of claims 56-75, wherein the agent or agents increase the amount and/or activity of CD8+ T cells infiltrating a tumor comprising the cancer cells.

77. The method of any one of claims 56-76, wherein the agent or agents a) increase the amount and/or activity of M1 macrophages infiltrating a tumor comprising the cancer cells and/or b) decrease the amount and/or activity of M2 macrophages infiltrating a tumor comprising the cancer cells.

78. The method of any one of claims 56-77, further comprising administering to the subject at least one additional therapy or regimen for treating the cancer.

79. The method of any one of claims 51-63, wherein the therapy is administered before, concurrently with, or after the agent.

80. A method of identifying monocytes and/or macrophages that can increase an inflammatory phenotype thereof by modulating at least one target comprising:
a) determining the copy number, amount, and/or activity of at least one target listed in Table 1 and/or Table 2 from the monocytes and/or macrophages;
b) determining the copy number, amount, and/or activity of the at least one target in a control; and
c) comparing the copy number, amount, and/or activity of the at least one target detected in steps a) and b);
wherein the presence of, or an increase in, the copy number, amount, and/or activity of, the at least one target listed in Table 1 and/or the absence of, or a decrease in, the copy number, amount, and/or activity of, the at least one target listed in Table 2, in the monocytes and/or macrophages relative to the control copy number, amount, and/or activity of the at least one target indicates that the monocytes and/or macrophages can increase the inflammatory phenotype thereof by modulating the at least one target.

81. The method of claim 80, further comprising contacting the cells with, recommending, prescribing, or administering an agent that modulates the at least one target listed in Table 1 and/or Table 2.

82. The method of claim 80, further comprising contacting the cells with, recommending, prescribing, or administering cancer therapy other than an agent that modulates the one or more targets listed in Table 1 and/or Table 2 if the subject is determined not to benefit from increasing an inflammatory phenotype by modulating the one or more targets.

83. The method of claim 81 or 82, further comprising contacting the cells with and/or administering at least one additional agent that increases an immune response

84. The method of claim 83, wherein the additional agent is selected from the group consisting of targeted therapy, chemotherapy, radiation therapy, and/or hormonal therapy.

85. The method of any one of claims 80-84, wherein the control is from a member of the same species to which the subject belongs.

86. The method of any one of claims 80-85, wherein the control is a sample comprising cells.

87. The method of any one of claims 80-86, wherein the subject is afflicted with a cancer.

88. The method of any one of claims 80-87, wherein the control is a cancer sample from the subject.

89. The method of any one of claims 80-87, wherein the control is a non-cancer sample from the subject.

90. A method of identifying monocytes and/or macrophages that can decrease an inflammatory phenotype thereof by modulating at least one target comprising:
a) determining the copy number, amount, and/or activity of at least one target listed in Table 1 and/or Table 2 from the monocytes and/or macrophages;
b) determining the copy number, amount, and/or activity of the at least one target in a control; and
c) comparing the copy number, amount, and/or activity of the at least one target detected in steps a) and b);
wherein the absence of, or a decrease in, the copy number, amount, and/or activity of, the at least one target listed in Table 1 and/or the presence of, or an increase in, the copy number, amount, and/or activity of, the at least one target listed in Table 2, in the monocytes and/or macrophages relative to the control copy number, amount, and/or activity of the at least one target indicates that the monocytes and/or macrophages that can decrease the inflammatory phenotype thereof by modulating the at least one target.

91. The method of claim 90, further comprising contacting the monocytes and/or macrophages with, recommending, prescribing, or administering an agent or agents that modulate the one or more targets listed in Table 1 and/or Table 2.

92. The method of claim 91, further comprising contacting the monocytes and/or macrophages with, recommending, prescribing, or administering cancer therapy other than an agent or agents that modulate the one or more targets listed in Table 1 and/or Table 2 if the subject is determined not to benefit from decreasing an inflammatory phenotype by modulating the at least one target.

93. The method of claim 91 or 92, further comprising contacting the monocytes and/or macrophages with and/or administering at least one additional agent that decreases an immune response.

94. The method of any one of claims 90-93, wherein the control is from a member of the same species to which the subject belongs.

95. The method of any one of claims 90-94, wherein the control is a sample comprising cells.

96. The method of any one of claims 90-95, wherein the subject is afflicted with a cancer.

97. The method of any one of claims 90-96, wherein the control is a cancer sample from the subject.

98. The method of any one of claims 90-96, wherein the control is a non-cancer sample from the subject.

99. A method for predicting the clinical outcome of a subject afflicted with a cancer, the method comprising:
a) determining the copy number, amount, and/or activity of at least one target listed in Table 1 and/or Table 2 from monocytes and/or macrophages from the subject;
b) determining the copy number, amount, and/or activity of the at least one target from a control having a poor clinical outcome; and
c) comparing the copy number, amount, and/or activity of the at least one target in the subject sample and in the sample from the control subject;
wherein the presence of, or an increase in, the copy number, amount, and/or activity of, the at least one target listed in Table 1 and/or the absence of, or a decrease in, the copy number, amount, and/or activity of, the at least one target listed in Table 2, from the monocytes and/or macrophages from the subject as compared to the copy number, amount and/or activity in the control, indicates that the subject does not have a poor clinical outcome.

100. A method for monitoring the inflammatory phenotype of monocytes and/or macrophages in a subject, the method comprising:
a) detecting in a first subject sample at a first point in time the copy number, amount, and/or or activity of at least one target listed in Table 1 and/or Table 2 from monocytes and/or macrophages from the subject;
b) repeating step a) using a subsequent sample comprising monocytes and/or macrophages obtained at a subsequent point in time; and
c) comparing the amount or activity of at least one target listed in Table 1 and/or Table 2 detected in steps a) and b),
wherein the absence of, or a decrease in, the copy number, amount, and/or activity of, the at least one target listed in Table 1 and/or the presence of, or an increase in, the copy number, amount, and/or activity of, the at least one target listed in Table 2, from the monocytes and/or macrophages from the subsequent sample as compared to the copy number, amount and/or activity from the monocytes and/or macrophages from the first sample indicates that the subject's monocytes and/or macrophages have an upregulated inflammatory phenotype; or
wherein the presence of, or an increase in, the copy number, amount, and/or activity of, the at least one target listed in Table 1 and/or the absence of, or a decrease in, the copy number, amount, and/or activity of, the at least one target listed in Table 2, from the monocytes and/or macrophages from the subsequent sample as compared to the copy number, amount and/or activity from the monocytes and/or macrophages from the first samples indicates that the subject's monocytes and/or macrophages have a downregulated inflammatory phenotype.

101. The method of claim 100, wherein the first and/or at least one subsequent sample comprises monocytes and/or macrophages that are cultured in vitro.

102. The method of claim 100, wherein the first and/or at least one subsequent sample comprises monocytes and/or macrophages that are not cultured in vitro.

103. The method of any one of claims 100-102, wherein the first and/or at least one subsequent sample is a portion of a single sample or pooled samples obtained from the subject.

104. The method of any one of claims 100-103, wherein the sample comprises blood, serum, peritumoral tissue, and/or intratumoral tissue obtained from the subject.

105. A method of assessing the efficacy of an agent for increasing an inflammatory phenotype of monocytes and/or macrophages in a subject, comprising:
a) detecting in a subject sample comprising monocytes and/or macrophages at a first point in time i) the copy number, amount, and/or or activity of at least one target listed in Table 1 and/or Table 2 in or on the monocytes and/or macrophages and/or ii) an inflammatory phenotype of the monocytes and/or macrophages;
b) repeating step a) during at least one subsequent point in time after the monocytes and/or macrophages are contacted with the agent; and
c) comparing the value of i) and/or ii) detected in steps a) and b), wherein the absence of, or a decrease in, the copy number, amount, and/or activity of, the at least one target listed in Table 1 and/or the presence of, or an increase in, the copy number, amount, and/or activity of, the at least one target listed in Table 2, and/or an increase in ii) in the subsequent sample as compared to the copy number, amount, and/or activity in the sample at the first point in time, indicates that the agent increases the inflammatory phenotype of monocytes and/or macrophages in the subject.

106. The method of claim 105, wherein the monocytes and/or macrophages contacted with the agent are comprised within a population of cells and the agent increases the number of Type 1 and/or M1 macrophages in the population of cells.

107. The method of claim 105 or 106, wherein the monocytes and/or macrophages contacted with the agent are comprised within a population of cells and the agent decreases the number of Type 2 and/or M2 macrophages in the population of cells.

108. A method of assessing the efficacy of an agent for decreasing an inflammatory phenotype of monocytes and/or macrophages, comprising:
a) detecting in a subject sample comprising monocytes and/or macrophages at a first point in time i) the copy number, amount, and/or or activity of at least one target listed in Table 1 and/or Table 2 in or on the monocytes and/or macrophages and/or ii) an inflammatory phenotype of the monocytes and/or macrophages;
b) repeating step a) during at least one subsequent point in time after the monocytes and/or macrophages are contacted with the agent; and
c) comparing the value of i) and/or ii) detected in steps a) and b), wherein the presence of, or an increase in, the copy number, amount, and/or activity of, the at least one target listed in Table 1 and/or the absence of, or a decrease in, the copy number, amount, and/or activity of, the at least one target listed in Table 2, and/or a decrease in ii) in the subsequent sample as compared to the copy number, amount, and/or activity in the sample at the first point in time, indicates that the agent decreases the inflammatory phenotype of monocytes and/or macrophages in the subject.

109. The method of claim 108, wherein the monocytes and/or macrophages contacted with the agent are comprised within a population of cells and the agent selectively decreases the number of Type 1 and/or M1 macrophages in the population of cells.

110. The method of claim 108 or 109, wherein the monocytes and/or macrophages contacted with the agent are comprised within a population of cells and the agent selectively increases the number of Type 2 and/or M2 macrophages in the population of cells.

111. The method of any one of claims 105-110, wherein the monocytes and/or macrophages are contacted in vitro or ex vivo.

112. The method of claim 111, wherein the monocytes and/or macrophages are primary monocytes and/or primary macrophages.

113. The method of claim 111 or 112, wherein the monocytes and/or macrophages are purified and/or cultured prior to contact with the agent.

114. The method of any one of claims 105-110, wherein the monocytes and/or macrophages are contacted in vivo.

115. The method of claim 114, wherein the monocytes and/or macrophages are contacted in vivo by systemic, peritumoral, or intratumoral administration of the agent.

116. The method of claim 114 or 115, wherein the monocytes and/or macrophages are contacted in a tissue microenvironment.

117. The method of any one of claims 105-116, further comprising contacting the monocytes and/or macrophages with at least one immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus.

118. The method of any one of claims 105-117, wherein the subject is a mammal.

119. The method of claim 118, wherein the mammal is a non-human animal model or a human.

120. A method of assessing the efficacy of an agent for treating a cancer in a subject, comprising:
a) detecting in a subject sample comprising monocytes and/or macrophages at a first point in time i) the copy number, amount, and/or or activity of at least one target listed in Table 1 and/or Table 2 in or on monocytes and/or macrophages and/or ii) an inflammatory phenotype of the monocytes and/or macrophages;
b) repeating step a) during at least one subsequent point in time after administration of the agent; and
c) comparing the value of i) and/or ii) detected in steps a) and b), wherein the absence of, or a decrease in, the copy number, amount, and/or activity of, the at least one target listed in Table 1 and/or the presence of, or an increase in, the copy number, amount, and/or activity of, the at least one target listed in Table 2, and/or an increase in ii) in or on the monocytes and/or macrophages of the subject sample at the subsequent point in time as compared to the copy number, amount, and/or activity in or on the monocytes and/or macrophages of the subject sample at the first point in time, indicates that the agent treats the cancer in the subject.

121. The method of claim 120, wherein between the first point in time and the subsequent point in time, the subject has undergone treatment, completed treatment, and/or is in remission for the cancer.

122. The method of claim 120 or 121, wherein the first and/or at least one subsequent sample is selected from the group consisting of ex vivo and in vivo samples.

123. The method of any one of claims 120-122, wherein the first and/or at least one subsequent sample is obtained from a non-human animal model of the cancer.

124. The method of any one of claims 120-123, wherein the first and/or at least one subsequent sample is a portion of a single sample or pooled samples obtained from the subject.

125. The method of any one of claims 120-124, wherein the sample comprises cells, serum, peritumoral tissue, and/or intratumoral tissue obtained from the subject.

126. A method for screening for agents that sensitize cancer cells to cytotoxic T cell-mediated killing and/or immune checkpoint therapy comprising
a) contacting cancer cells with cytotoxic T cells and/or immune checkpoint therapy in the presence of monocytes and/or macrophages contacted with i) at least one agent that decreases the copy number, amount, and/or activity of at least one target listed in Table 1 and/or ii) at least one agent that increases the copy number, amount, and/or activity of the at least one target listed in Table 2;
b) contacting cancer cells with cytotoxic T cells and/or immune checkpoint therapy in the presence of control monocytes and/or macrophages that are not contacted with the at least one agent or agents; and
c) identifying agents that sensitize cancer cells to cytotoxic T cell-mediated killing and/or immune checkpoint therapy by identifying agents that increase cytotoxic T cell-mediated killing and/or immune checkpoint therapy efficacy in a) compared to b).

127. A method for screening for agents that sensitize cancer cells to cytotoxic T cell-mediated killing and/or immune checkpoint therapy comprising
a) contacting cancer cells with cytotoxic T cells and/or immune checkpoint therapy in the presence of monocytes and/or macrophages engineered to decrease the copy number, amount, and/or activity of at least one target listed in Table 1 and/or ii) engineered to increase the copy number, amount, and/or activity of the at least one target listed in Table 2;
b) contacting cancer cells with cytotoxic T cells and/or immune checkpoint therapy in the presence of control monocytes and/or macrophages; and
c) identifying agents that sensitize cancer cells to cytotoxic T cell-mediated killing and/or immune checkpoint therapy by identifying agents that increase cytotoxic T cell-mediated killing and/or immune checkpoint therapy efficacy in a) compared to b).

128. The method of claim 126 or 127, wherein the step of contacting occurs in vivo, ex vivo, or in vitro.

129. The method of any one of claims 120-128, further comprising determining a reduction in i) the number of proliferating cells in the cancer and/or ii) a reduction in the volume or size of a tumor comprising the cancer cells.

130. The method of any one of claims 120-129, further comprising determining i) an increased number of CD8+ T cells and/or ii) an increased number of Type 1 and/or M1 macrophages infiltrating a tumor comprising the cancer cells.

131. The method of any one of claims 120-130, further comprising determining responsiveness to the agent that modulates the at least one target listed in Table 1 and/or Table 2 measured by at least one criterion selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

132. The method of any one of claims 120-131, further comprising contacting the cancer cells with at least one additional cancer therapeutic agent or regimen.

133. The method or composition of any one of claims 1-132 wherein the agent or agents further comprise a lipid or lipidoid.

134. The method or composition of claim 133, wherein the lipidoid is of Formula (VI): 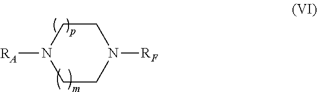
wherein:
p is an integer between 1 and 3, inclusive;
m is an integer between 1 and 3, inclusive;
R_{A} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl; 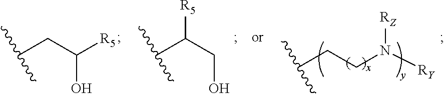
R_{F} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl; 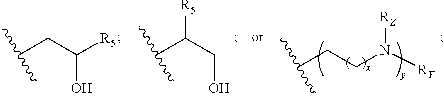
each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;
wherein, at least one of R_{A}, R_{F}, R_{Y}, and R_{Z} is 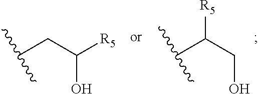
each occurrence of x is an integer between 1 and 10, inclusive;
each occurrence of y is an integer between 1 and 10, inclusive;
each occurrence of R_{Y} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl; 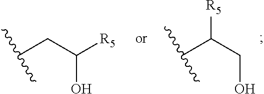
each occurrence of R_{Z} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl; 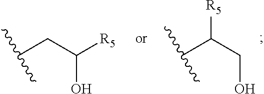
or a pharmaceutically acceptable salt thereof.

135. The method or composition of claim 134, wherein p is 1.

136. The method or composition of claim 134 or 135, wherein m is 1.

137. The method or composition of any one of claims 134-136, wherein each of p and m are 1.

138. The method or composition of any one of claims 134-137, wherein R_{F} is

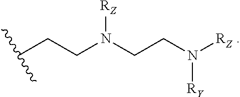

139. The method or composition of any one of claims 134-138, wherein R_{A} is

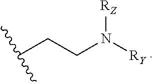

140. The method or composition of claim 134, wherein the compound of Formula (VI) is of the formula: 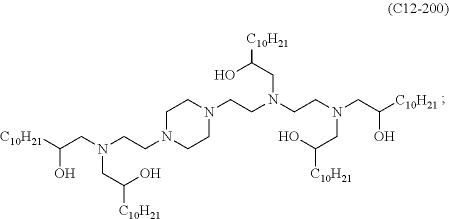
or a salt thereof.

141. The method or composition of any one of claims 134-140, wherein the composition is in the form a lipid nanoparticle.

142. The method or composition of claim 141, wherein the lipid nanoparticle comprises about 1.0% to about 60.0% by mole of C12-200.

143. The method or composition of claim 141 or 142, wherein the lipid nanoparticle further comprises one or more co-lipids.

144. The method or composition of claim 143, wherein each co-lipid is selected from disteroylphosphatidyl choline (DSPC), cholesterol, and DMG-PEG.

145. The method or composition of claim 144, wherein the concentration of DSPC is about 1.0% to about 20.0% by mole.

146. The method or composition of claim 144 or 145, wherein the concentration of cholesterol is about 10.0% to about 50.0% by mole.

147. The method or composition of any one of claims 144-146, wherein the concentration of DMG-PEG is about 0.1% to about 5.0% by mole.

148. The method or composition of any one of claims 136-147, wherein DSPC is present a concentration of about 1.0% to about 20.0% by mole; cholesterol is present at a concentration of about 10.0% to about 50.0% by mole; and DMG-PEG is present a concentration of about 0.1% to about 5.0% by mole.

149. The method or composition of any one of claims 1-148, wherein the agent is in a pharmaceutically acceptable formulation, optionally wherein the pharmaceutically acceptable formulation is substantially endotoxin-free and/or has less than about 1 EU/mg protein.

150. The method or composition of any one of claims 1-149, wherein the monocytes and/or macrophages having a modulated inflammatory phenotype exhibit one or more of the following:
a) modulated expression of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α);
b) modulated expression of CD206, CD163, CD16, CD53, VSIG4, PSGL-1 and/or IL-10;
c) modulated secretion of at least one cytokine selected from the group consisting of IL-1β, TNF-α, IL-12, IL-18, and IL-23;
d) modulated ratio of expression of IL-1β, IL-6, and/or TNF-α to expression of IL-10;
e) modulated CD8+ cytotoxic T cell activation;
f) modulated CD4+ helper T cell activity;
g) modulated NK cell activity;
h) modulated neutrophil activity;
i) modulated macrophage activity; and/or
j) modulated spindle-shaped morphology, flatness of appearance, and/or dendrite numbers, as assessed by microscopy.

151. The method or composition of any one of claims 1-150, wherein the cells and/or macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the cells and/or macrophages express or are determined to express at least one target selected from the group consisting of targets listed in Table 1 and/or Table 2.

152. The method or composition of any one of claims 1-151, wherein the at least one target listed in Table 1 is selected from the group consisting of human SIGLEC9, VSIG4, CD74, CD207, LRRC25, SELPLG, AIF1, CD84, IGSF6, CD48, CD33, LST1, TNFAIP8L2 (TIPE2), SPI1 (PU.1), LILRB2, CCR5, EVI2B, CLEC7A, TBXAS1, SIGLEC7, and DOCK2, or a fragment thereof.

153. The method or composition of any one of claims 1-152, wherein the at least one target listed in Table 2 is selected from the group consisting of human CD53, FERMT3, CD37, CXorf21, CD48, and CD84, or a fragment thereof.

154. The method or composition of any one of claims 1-153, wherein the cancer is a solid tumor that is infiltrated with macrophages, wherein the infiltrating macrophages represent at least about 5% of the mass, volume, and/or number of cells in the tumor or the tumor microenvironment, and/or wherein the cancer is selected from the group consisting of mesothelioma, kidney renal clear cell carcinoma, glioblastoma, lung adenocarcinoma, lung squamous cell carcinoma, pancreatic adenocarcinoma, breast invasive carcinoma, acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous, cystadenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, and uveal melanoma.

155. The method or composition of claim 154, wherein the macrophages comprise Type 1 macrophages, M1 macrophages, Type 2 macrophages, M2 macrophages, M2c macrophages, M2d macrophages, tumor-associated macrophages (TAM), CD11b+ cells, CD14+ cells, and/or CD11b+/CD14+ cells, optionally wherein the macrophages are TAMs and/or M2 macrophages.

156. The method or composition of claim 155, wherein the macrophages express or are determined to express one or more targets selected from the group consisting of targets listed in Table 1 and/or Table 2.

157. The method or composition of claim 156, wherein the at least one target listed in Table 1 is selected from the group consisting of human SIGLEC9, VSIG4, CD74, CD207, LRRC25, SELPLG, AIF1, CD84, IGSF6, CD48, CD33, LST1, TNFAIP8L2 (TIPE2), SPI (PU.1), LILRB2, CCR5, EVI2B, CLEC7A, TBXAS1, SIGLEC7, and DOCK2, or a fragment thereof.

158. The method or composition of claim 156 or 157, wherein the at least one target listed in Table 2 is selected from the group consisting of human CD53, FERMT3, CD37, CXorf21, CD48, and CD84, or a fragment thereof.

159. The method or composition of any one of claims 1-158, wherein the monocytes and/or macrophages are primary monocytes and/or primary macrophages.

160. The method or composition of any one of claims 1-159, wherein the monocytes and/or macrophages are comprised within a tissue microenvironment.

161. The method or composition of anyone of claims 1-160, wherein the monocytes and/or macrophages are comprised within a human tumor model or an animal model of cancer.

162. The method or composition of any one of claims 1-161, wherein the subject is a mammal.

163. The method or composition of claim 162, wherein the mammal is a human.

164. The method or composition of claim 163, wherein the human is afflicted with a cancer.
